(19) United States
(12) Patent Application Publication
(10) Pub. No.: US 2024/0262791 A1
(43) Pub. Date: Aug. 8, 2024

(54) BIOREACTIVE PROTEINS CONTAINING UNNATURAL AMINO ACIDS

(71) Applicant: The Regents of the University of California, Oakland, CA (US)

(72) Inventors: Lei Wang, San Francisco, CA (US); Wei Sun, San Francisco, CA (US); Paul C. Klauser, San Francisco, CA (US); Li Cao, San Francisco, CA (US); Bingchen Yu, San Francisco, CA (US); Nanxi Wang, San Francisco, CA (US); Shanshan Li, San Francisco, CA (US); Michael J. Evans, South San Francisco, CA (US)

(21) Appl. No.: 18/288,679

(22) PCT Filed: Apr. 28, 2022
(86) PCT No.: PCT/US2022/026708

§ 371 Date: Oct. 27, 2023

Related U.S. Application Data
(60) Provisional application No. 63/301,940, filed on Jan. 21, 2022.
(60) Provisional application No. 63/289,573, filed on Dec. 14, 2021.
(60) Provisional application No. 63/238,360, filed on Aug. 30, 2021.
(60) Provisional application No. 63/180,827, filed on Apr. 28, 2021.
(60) Provisional application No. 63/318,960, filed on Mar. 11, 2022.

(51) Int. Cl. C07C 305/26; C07C 309/87; C07C 309/88; C07C 309/89

(52) U.S. Cl. CPC C07C 305/26; C07C 309/87; C07C 309/88; C07C 309/89

(57) ABSTRACT

Provided herein are inter alia, unnatural amino acids based on fluorosulfonyloxybenzoyl-L-lysine FSK, proteins comprising unnatural amino acids, nanobodies comprising unnatural amino acids based on fluorosulfate-L-tyrosine FSY, meta-FSY and FFY within CDR1, CDR2, or CDR3, biomolecule conjugates, and methods of making the proteins and biomolecule conjugates.

### CROSS-REFERENCE TO RELATED APPLICATIONS

This application claims the benefit of priority to U.S. Application No. 63/318,960 filed Mar. 11, 2022, U.S. Application No. 63/301,940 filed Jan. 21, 2022, U.S. Application No. 63/289,573 filed Dec. 14, 2021, U.S. Application No. 63/238,360 filed Aug. 30, 2021, and U.S. Application No. 63/180,827 filed Apr. 28, 2021, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under grants R01 CA258300 and R01 GM118384 awarded by The National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-713001WO_SL_ST25.txt, created on Apr. 21, 2022, 412,921 bytes, machine format IBM-PC, MS Windows operating system, is hereby incorporated by reference.

### BACKGROUND

Introducing new chemical bonds into proteins provides innovative avenues for manipulating protein structure and function. Unnatural amino acids (Uaas) containing diverse latent bioreactive functional groups have recently been introduced into proteins via genetic code expansion. This offers an exquisite tool not only to study cellular protein interactions but also create novel protein-based therapeutics. SuFEx click chemistry via the latent aryl fluorosulfate group has demonstrated value in aiding modular organic synthesis, chemical biology, and drug development. As set forth in US Publication No. 2021/0002325, the inventors incorporated fluorosulfate-L-tyrosine (FSY) into proteins for protein crosslinking and generating covalent protein drugs. There is a need in the art, inter alia, for new and other unnatural amino acids that can be used for protein identification, drug target discovery, or biotherapeutics. Provided herein are solutions to these and other needs in the art.

### SUMMARY

Provided herein are nanobodies comprising an unnatural amino acid within CDR1, CDR2, or CDR3. Provided herein are nanobodies comprising an unnatural amino acid within CDR1, CDR2, or CDR3, wherein the unnatural amino acid is FSK. Provided herein are nanobodies comprising an unnatural amino acid within CDR1, CDR2, or CDR3, wherein the unnatural amino acid is FSY. Provided herein are nanobodies comprising an unnatural amino acid within CDR1, CDR2, or CDR3, wherein the unnatural amino acid is meta-FSY. Provided herein are nanobodies comprising an unnatural amino acid within CDR1, CDR2, or CDR3, wherein the unnatural amino acid is FFY. Provided herein are nanobodies comprising an unnatural amino acid within CDR1, CDR2, or CDR3, wherein the unnatural amino acid comprises a side chain of Formula (II), Formula (V), or Formula (VIII).

Provided herein are compounds of Formula (I) or a stereoisomer thereof:

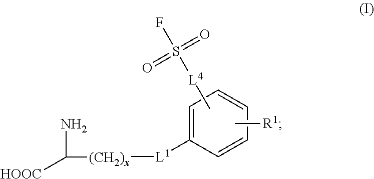

wherein the substituents are as defined herein.

Provided herein are RNA-binding protein comprises an unnatural amino acid, wherein the unnatural amino acid comprises a side chain of Formula (II):

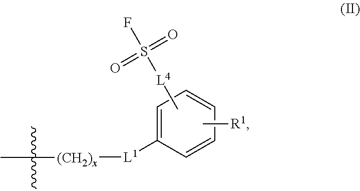

wherein the substituents are as defined herein. In embodiments, the RNA-binding protein is a CRISPR protein or a RNA chaperone.

Provided herein are biomolecule conjugates of Formula (III):

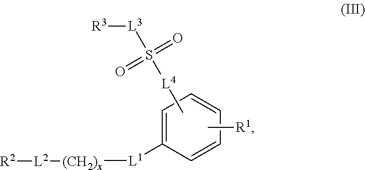

wherein R² is a RNA-binding protein moiety; R³ is a RNA moiety; and the remaining substituents are as defined herein. In embodiments, the RNA-binding protein is a CRISPR protein or a RNA chaperone.

Provided herein are compounds of Formula (IV):

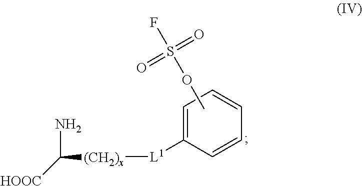

wherein —OS(═O)₂F is meta or ortho to the carbon atom linked to L¹; and x and L¹ are as defined herein.

Provided herein are proteins comprising an unnatural amino acid, wherein the unnatural amino acid comprises a side chain of Formula (V):

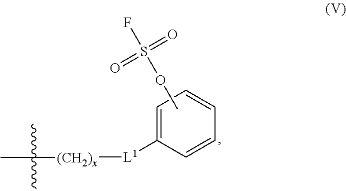

wherein —OS(═O)₂F is meta or ortho to the carbon atom linked to L¹; and x and L¹ are as defined herein.

Provided herein are biomolecule conjugates of Formula (VI):

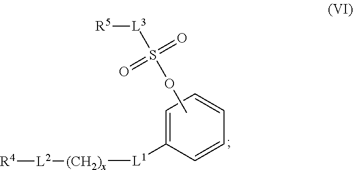

wherein —OS(═O)₂L³R⁵ is meta or ortho to the carbon atom linked to L¹; R⁴ and R⁵ are each independently a peptidyl moiety, a carbohydrate moiety, or a nucleic acid moiety; and x, L¹, L², and L³ are as defined herein. In embodiments, R⁴ is a peptidyl moiety and R⁵ is a peptidyl moiety comprising lysine, histidine, or tyrosine bonded to L³.

Provided herein are compounds of Formula (VII) or a stereoisomer thereof:

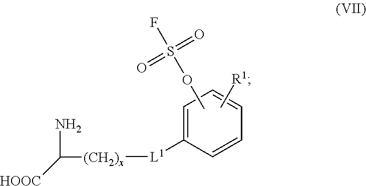

Wherein the substituents are as defined herein. The disclosure provides proteins comprising the compound of Formula (VII) and biomolecule conjugates comprising the compound of Formula (VII).

These and other embodiments of the disclosure are provided in detail herein.

### BRIEF DESCRIPTION OF THE DRAWINGS

FIGS. 1A-1I show that GECX-RNA enables FSY-incorporated dPsCas13b crosslinking target RNA in vitro. FIG. 1A: Scheme showing proximity-enabled SuFEx reaction between FSY and a nucleophilic group of RNA, which can be 2′-OH on ribose or amino group on base. FIG. 1B: Structure of Cas13-crRNA-target RNA ternary complex showing sites 133 and 1058 (yellow stick) chosen for FSY incorporations in dPsCas13b protein (PDB: 5XWP). FIG. 1C: Denaturing Urea-PAGE gel demonstrating dPsCas13b-133FSY crosslinked with the target RNA (ssRNA-1) with guidance of crRNA (crRNA-1). After incubation, samples were either directly separated on denaturing Urea-PAGE (w/o protease) or treated with protease K before separated on denaturing Urea-PAGE (w/protease). The Urea-gels were stained with SybrGold for fluorescent detection of RNA. FIG. 1D: Denaturing Urea-PAGE gel demonstrating crosslinking of target RNA (IRD680-ssRNA-1) required guidance of crRNA. dPsCas13b-WT or 133FSY proteins were incubated with different combinations of crRNA-1 and target RNA fluorescently labeled with IRD680 at 5′ end (IRD680-ssRNA-1). After incubation, samples were separated on denaturing Urea-PAGE. The gel was imaged by scanning IRD680 signal. FIG. 1E: Structure of BzoCas13b-crRNA binary complex showing positively charged amino acids (yellow stick) located on D-sheets 5 and 6 (magenta colored) for pre-crRNA cleavage. Target nucleotide of cleavage on pre-crRNA was shown as grey stick (PDB: 6AAY). FIG. 1F: Scheme of Cas13b processing pre-crRNA at the phosphodiester bond connecting two nucleotides located directly 3′-downstream of the hairpin repeat region. Red arrow indicate the cleavage site. FIG. 1G: Multiple sequence alignment of Cas13b proteins from different species (Bzo: Bergeyella zoohelcum, Psp: Prevotella sp. P5-125, Pgu: Porphyromonas gingivalis, Pbu: Prevotella buccae, and Ran: Riemerella anatipestifer) for β-sheets 5 and 6 for pre-crRNA cleavage. The secondary structure of BzoCas13b is shown above the sequence. (Ref 23). Identical and similar residues are highlighted in red and white boxes, respectively. Positive charged catalytic residues in BzoCas13b involved in the pre-crRNA cleavage on β-sheets 5 and 6 (450R, 452K, 459R) are marked with green stars on the bottom. Positive charged residues in PsCas13b located on β-sheets 5 and 6 (367K, 370K, 378R, 380R) are marked with purple squares. Multiple sequence alignment of full-length Cas13b proteins from different species is shown in FIG. 6. FIG. 1H: Denaturing urea-PAGE demonstrating the pre-crRNA cleavage by dPsCas13b-WT and dPsCas13b-Ala-mutants speculatively involved in the pre-crRNA processing. dPsCas13b-WT and dPsCas13b-Ala-mutants were incubated with pre-crRNA and then separated on denaturing urea-PAGE. The Urea-gel was stained with SybrGold for fluorescent detection of RNA. FIG. 1I: Denaturing urea-PAGE demonstrating no nucleotide bias for FSY crosslinking. dPsCas13b-380A or dPsCas13b-380FSY was incubated with pre-crRNAs containing different nucleotide compositions at cleavage site. Nucleotide sequences at cleavage sites (as NNN shown in (FIG. 1F)) were placed as AAA, UUU, CCC, or GGG in pre-crRNA-AAA, pre-crRNA-UUU, pre-crRNA-CCC, or pre-crRNA-GGG, respectively. After incubation, samples were separated on denaturing Urea-PAGE. The Urea-gels were stained with SybrGold for fluorescent detection of RNA.

FIGS. 2A-2F show that GECX-RNA enables FSY-incorporated Hfq proteins to crosslink target RNA in E. coli. FIG. 2A: Structure of E. coli Hfq bound to target RNA showing three chosen sites (Y25, I30, and T49) in yellow stick for FSY incorporation and the RNA in grey (PDB: 4HT8). FIG. 2B: Western blot analysis demonstrating FSY-incorporated Hfq proteins crosslinked with RNA molecules in E. coli cells. Hfq-FSY proteins were expressed in E. coli DH10B strain. Cell lysate samples were treated with or without RNase before loading, and an anti-His antibody was used to detect the 6×His tag appended at the C-terminus of expressed Hfq. FIG. 2C: Scheme of reverse transcription (RT) and quantitative-PCR (qPCR) of RNA crosslinked by Hfq. FIG. 2D: RT-qPCR analyses of Hfq co-purified RNA demonstrate that FSY-incorporated Hfq proteins crosslinked and enriched target RNA rpoS in E. coli cells. Hfq proteins (Hfq-WT and Hfq-FSY) were purified from E. coli cells, and RT-qPCR analysis was performed on co-purified RNA samples. Enrichment fold changes were calculated based on normalizations to input-RNA samples using mpB gene as reference. Control sample was cells without exogenous Hfq expression. Fold-changes of target RNAs in Hfq-FSY samples compared to Hfq-WT samples were shown. Error bars represent s.e.m.; n=3 independent biological replicates; * p<0.05; n.s., not significant; multiple t test. FIG. 2E: Scheme of GRIP. After protease K treatment, co-purified Hfq-crosslinked RNA were reversely transcribed by gene-specific RT primer, followed by RNA removal and ligation of a 3's cDNA adaptor containing a random-10mer at the ligation site. After ligation, PCR was performed with a primer pair, one targeting gene-specific region and the other targeting 3's cDNA adaptor region. Sequencing of the PCR product could identify the ligation sites, indicating RT terminating sites and the crosslinking sites (red triangle). FIG. 2F: Crosslinking sites identified from GRIP for rpoS RNA from Hfq-25FSY expressing E. coli cells. Red triangles, crosslinking sites of sequenced clones from Hfq-25FSY expressing E. coli cells, indicate that site 25 of Hfq directly binds with (AAN)4 elements of rpoS RNA. Two examples of sanger sequencing of clones from Hfq-25FSY sample were shown below.

FIGS. 3A-3B show that GECX-RNA enables FSY-incorporated dPsCas13b proteins to crosslink target RNA in mammalian cells. FIG. 3A: Scheme showing the procedures for quantification of RNA co-purified with dPsCas13b from mammalian cells. FIG. 3B: RT-qPCR analysis of dPsCas13b co-purified RNA showed that dPsCas13b-133FSY enriched more target RNA molecules than dPsCas13b-WT with the guidance of crRNA. Control samples had no crRNA plasmid transfected, while crACTB, crNEAT1-1, and crNEAT1-2 samples were transfected with distinct crRNA plasmids targeting ACTB mRNA or NEAT1 RNA. Bar chart showed the fold-changes of target RNAs in crRNA transfected samples compared to control samples (normalized to GAPDH RNA abundance). Error bars represent s.e.m.; n=2 independent biological replicates; * p<0.05; n.s., not significant; multiple t test.

FIGS. 4A-4I show that SFY allows crosslinking of His, Tyr, Lys residues in protein and of RNA in cells. FIG. 4A: Structure of SFY. FIG. 4B: Fluorescence confocal images HEK293 cells expressing EGFP(40TAG) gene and the Mm-tRNA^{Pyl}/MmSFYRS with and without 1 mM SFY. FIG. 4C: Flow cytometric analysis of SFY incorporation into EGFP(40TAG) in HEK293 cells using Ma-tRNA^{Pyl}/MaSFYRS. FIG. 4D: Structure of Afb-Z complex showing two proximal sites for SFY and target residue X incorporation. FIGS. 4E-4F: Analysis of crosslinking of Afb(24SFY) with MBP-Z(7X) in E. coli cells. Western blot of E. coli cell lysate (FIG. 4E); SDS-PAGE of proteins His-tag purified from E. coli (FIG. 4F). Maltose binding protein (MBP) was fused to the N-terminus of Z protein to better separate Z from Afb in size. FIG. 4G: Crystal structure of E. coli GST (PDB: 1A0F) showing site 103 and 107 at the dimer interface. FIG. 4H: Western blot analysis of lysate of HEK293T cells expressing GST(103SFY-107X). X is the target residue indicated. FIG. 4I: Western blot analysis E. coli cells expressing Hfq with SFY incorporated at site 25 or 49. Cell lysate samples were treated with or without RNase before loading, and an anti-His antibody was used to detect the 6×His tag appended at the C-terminus of expressed Hfq. Star indicates a cross-linked band.

FIGS. 5A-5E show GRIP in mammalian cell for in vivo detection of m6A on RNA with single-nucleotide resolution. FIG. 5A: Scheme showing the principle of using GRIP to detect RNA modifications in vivo, using m6A as an example. A reader protein recognizing the RNA modification is expressed in cells, with a latent bioreactive Uaa incorporated near the recognition site to crosslink bound RNA for identification. GRIP identifies the crosslinking site, and the RNA modification will be next to the crosslink site. FIG. 5B: Structure of YTH domain (from human YTHDF1) binding with m6A nucleotide (PDB: 4RCJ). Tyr397, the site chosen for incorporation of SFY is shown in grey stick. RNA is colored in yellow and YTH protein in green. FIG. 5C: Scheme of GRIP procedures for in vivo m6A detection. FIGS. 5D-5E: m6A sites identified from JUN mRNA. Red triangles showed crosslinking sites of sequenced clones from YTH-397SFY expressing cells. Blue arrows showed the m6A site indicated from sequenced clone result. Grey triangle showed m6A site reported from previous study. (Ref 45). Examples of clone sequencing result were shown below.

FIG. 6 shows multiple sequence alignment of Cas13b proteins from different species. Sequence alignment of BzoCas13b (Bergeyella zoohelcum Cas13b), PspCas13b (Prevotella sp. P5-125), PguCas13b (Porphyromonas gingivalis Cas13b), PbuCas13b (Prevotella buccae Cas13b) and RanCas13b (Riemerella anatipestifer Cas13b) was generated using Clustal Omega and the figure was prepared using ESPript (http://espript.ibcp.fr). The secondary structure of BzoCas13b is shown above the sequence. Zhang et al, Cell Res. 28, 1198-1201 (2018). Identical and similar residues are highlighted in red and white boxes, respectively. Positive charged catalytic residues in BzoCas13b involved in the pre-crRNA cleavage on β-sheets 5 and 6 (450R, 452K, 459R) are marked with green stars on the bottom. Positive charged residues in PspCas13b located on β-sheets 5 and 6 (367K, 370K, 378R, 380R) are marked with purple squares.

FIGS. 7A-7D. FIG. 7A: Western blot of Hfq proteins for cell lysates and purified samples. Western blot was performed with anti-His antibody. FIG. 7B: RT-qPCR analysis on rpoS RNA expression levels in E. coli cells with different Hfq expressions. E. coli cells exogenously expressing different Hfq proteins (WT protein or Hfq-FSY proteins) had similar up-regulation of rpoS RNA expression. Gene expression fold-changes were calculated based on normalizations to control samples using rnpB gene as reference. Control sample was without exogenous expression of Hfq protein. Other samples are with exogenous expression of different Hfq proteins. Bar chart showed the fold-changes of rpoS RNA in Hfq exogenously expressing samples compared to the control sample. Error bars represent s.e.m.; n=3 independent biological replicates. * p<0.05; ** p<0.01; *** p<0.001; n.s., not significant; multiple t test. FIG. 7C: Agarose gel analysis of PCR products from Hfq GRIP for region of rpoS RNA. FIG. 7D: GRIP results demonstrate that site 25 of Hfq directly binds with (ARN)4 elements of ptsG mRNA. Red triangles indicate cross-linking sites identified from Hfq GRIP for ptsG mRNA from Hfq-25FSY expressing E. coli cells. Two representative examples of sanger sequencing for clones from Hfq-25FSY sample were shown below.

FIGS. 8A-8B are western blot analysis demonstrating the successful expression and immunoprecipitation of dCas13b proteins in HEK293 cells. dCas13b could be detected in input cell lysates (FIG. 8A) and IP samples (FIG. 8B). Western blot was performed with anti-HA antibody.

FIG. 9A-9B are flow cytometric analysis of SFY incorporation into EGFP in HEK293 cells. FIG. 9A: SFY incorporation into EGFP(182TAG) in HEK293 cells using Ma-tRNA^{Pyl}/MaSFYRS. FIG. 9B: SFY incorporation into EGFP(40TAG) or EGFP(182TAG) in HEK293 cells using Mm-tRNA^{Pyl}/MmSFYRS.

FIG. 10 is a cell viability assay for HEK293T incubated with various concentrations of SFY for 24 h or 48 h. Error bars represent s.e.m.; n=3 independent tests.

FIG. 11A-11C. FIG. 11A: Western blot analysis demonstrating the successful expression and immunoprecipitation of YTH-WT and YTH-397SFY proteins in HEK293 cells. An anti-HA antibody was used for detection. FIGS. 11B-11E: Agarose gel analysis of PCR products from YTH GRIP PCR for regions of JUN (FIG. 11C), ACTB (FIG. 11D), and DICER1 (FIG. 11E) mRNAs. FIGS. 11F-11H: m6A sites identified from YTH GRIP for region of ACTB and DICER1 mRNAs. Red triangles showed ligation sites of sequenced clones from YTH-397SFY expressing cells. Blue arrows showed the m6A site indicated from sequenced clone results. Grey triangles showed m6A site reported from Tang et al, Nucleic Acids Res. 49, D134-D143 (2020). Examples of clone sequencing result were shown below.

FIGS. 12A-12C: Genetic incorporation of mFSY into proteins in mammalian cells. FIG. 12A: FACS analysis of mFSY incorporation into HeLa-EGFP (182TAG) cells. Negative control cells were either not transfected with any plasmid or were not treated with mFSY. FACS data is representative of three biological replicates. FIG. 12B: Bar graph showing total EGFP fluorescence percentage from FACS data. Error bar: s.d., n=3. FIG. 12C: Fluorescence microscopy and brightfield images of HeLa-EGFP (182TAG) reporter cells under two conditions: no mFSY added or 1 mM mFSY added. Fluorescence is only seen after the addition of mFSY.

FIGS. 13A-13C: mFSY facilitates crosslinking between affibody dimer dZ_{HER2} and HER2 receptor. FIG. 13A: Structure of affibody Z_{HER2} (pink) in complex with the extracellular domain of HER2 (silver) (PDB code: 3MZW), showing positions D36 and D37 (highlighted green) on the affibody in proximity to H490 (purple) of HER2. FIG. 13B: Western blot analysis of in vitro crosslinking between HER2 extracellular domain and dZ_{HER2}-36TAG mutants incorporating either FSY or mFSY. Crosslinking band, increasing over time, is indicated. FIG. 13C: Western blot analysis of in vitro crosslinking between HER2 and dZ_{HER2}-37TAG mutants, dZ_{HER2}-37FSY and dZ_{HER2}-37mFSY. Crosslinking band intensity increases over time for all mutants. Represented time points: 0.5h, 2h, 4h, and 24h.

FIGS. 14A-14B: Incorporation of mFSY into TrasFab enables first shown instance of Fab-receptor crosslinking with HER2. FIG. 14A: Structure of Trastuzumab Fab (TrasFab, gold and mint) in complex with HER2 extracellular domain (purple) (PDB code: 1N8Z). Residues S50 and Y92 (blue) of the TrasFab light chain (LC) are shown in proximity to targeted residue K593 on HER2. FIG. 14B: SDS-PAGE analysis of in vitro covalent crosslinking at different time points between TrasFab(LC) mutants and HER2 extracellular domain. TrasFab(LC)-92FSY and TrasFab(LC)-92mFSY show efficient, time-dependent crosslinking. TrasFab(LC)-50mFSY shows less robust, but still detectable, crosslinking. Represented time points: 0.5h, 2h, 4h, and 24h.

FIGS. 15A-15B: Nb_{EGFR}-Q116mFSY robustly crosslinks EGFR when compared to the Q116FSY mutant. FIG. 15A: Structure of Nb_{EGFR} (mint) in complex with EGFR (gold) (PDB code: 4KRL), showing the qite Q116 on Nb_{EGFR} in proximity to H409 (purple) of EGFR. FIG. 15B: Western blot analysis of WT Nb_{EGFR}, and Q116FSY and Q116mFSY Nb_{EGFR} mutants incubated with EGFR receptor. Crosslinking bands can be seen for Nb_{EGFR}-116FSY and Nb_{EGFR}-116mFSY samples with Uaa added, but not WT Nb_{EGFR} or samples without Uaa added. Nb_{EGFR} is alternatively referred to as nanobody 7D12 and has SEQ ID NO:154.

FIGS. 16A-16B: NRG1b-A53mFSY effectively crosslinks HER3 over the FSY mutant. FIG. 16A: Structure of Neuregulin 1b (NRG1b, pink) bound to the extracellular domain of HER3 (PDB code: 7MN5). Site A53 is highlighted in blue and proximal nucleophilic residues on HER3 are also shown (sites K479 and H480). FIG. 16B: Western blot analysis of A53FSY and A53mFSY mutants incubated with or without HER3 extracellular domain. A crosslinking band is seen in the lane containing A53mFSY incubated with HER3 extracellular domain, and not in any other lane.

FIGS. 17A-17B: mFSY synthetase efficiently and selectively incorporates mFSY into proteins. FIG. 17A: Selection plates for mFSY synthetase. FIG. 17B: mFSY PyIRS synthetase (mFSYRS) encoded into pEvol plasmid efficiently incorporates mFSY into EGFP-182TAG approximately 100× the rate of misincorporation of native amino acids.

FIGS. 18A-18B: mFSY incorporation into nanobody Nb_{HER2} leads to detectable crosslinking between the covalent nanobody and HER2 receptor. FIG. 18A: Structure of Nb_{Her2} (pink) in complex with HER2 receptor (purple) (PDB code: 5MY6). Residue Y37 (green) of Nb_{HER2} is shown in proximity to residue Y112 of HER2. FIG. 18B: Incorporation of mFSY at site Y37 of Nb_{HER2} leads to detectable in vitro crosslinking of the nanobody with HER2 extracellular domain, in a time-dependent manner. Incorporation of FSY into the same site shows negligible crosslinking. Represented time points: 2h, 4h, and 24h. Nb_{HER2} is equivalent to 2rs15d or nanobody 2rs15d and is represented by SEQ ID NO:66.

FIGS. 19A-19C provide evidence of the discovery of F-FSY as latent bioreactive unnatural amino acid (Uaa) for protein-protein cross-linking. FIG. 19A: Structure of FSY and F-FSY; FIG. 19B: Incorporation of F-FSY into EGFR by FSYRS, n=3, values are mean+SD; FIG. 19C: SDS-PAGE of cross-linking between Afb7X with MBP-Z(24F-FSY).

FIGS. 20A-20C provide evidence of the crosslinking of mNb6 and the SARS-CoV-2 spike protein. FIG. 20A: Structure of mNb6 in complex with S protein; FIG. 20B: Kinetics study of cross-link between mNb6(108FSY) and S protein; FIG. 20C: Kinetics study of cross-link between mNb6(108F-FSY) and S protein.

FIGS. 21A-21D show that Nb_{HER2} (D54FSY) covalently binds to HER2 via incorporation of latent bioreactive Uaa. FIG. 21A: Schematic demonstrating the proximity-enabled reactivity, where the covalent complex forms once the nanobody is bound. FSY Uaa forms an irreversible covalent bond with lysine via click chemistry SuFEx. FIG. 21B: Crystal structure of NbHER2 bound to HER2 ECD (PDB: 5MY6). Shown in stick is the FSY incorporation site (D54) and the amino acid residue it targets (K150). FIG. 21C: NbHER2 (D54FSY) crosslinking assay shown was done at 37° C. over 4 h. The covalent complex forms only when NbHER2 (D54FSY) is incubated with HER2 ECD. FIG. 21D: Kinetics of NbHER2 (D54FSY) crosslinking with HER2. Using densitometry, the concentration of NbHER2 (D54FSY) at different timepoints were measured and 1/[NbHER2 (D54FSY)] was plotted against time. Linear regression of the data yielded a second-order rate constant of 34154±1921 M-1min-1 (mean±s.d.). Error bars represent s.d., n=3.

FIGS. 22A-22C NbHER2 (D54FSY) Covalently Crosslinks on NCI-N87 cells surface and shows dramatically improved tumor retention compared to NbHER2(WT). FIG. 22A: NbHER2(D54FSY) covalently crosslinks HER2 on NCI-N87 cell surface after 3 h incubation. No crosslinking was observed with NbHER2(WT) or PBS control. FIG. 22B: Representative decay-corrected coronal and transverse PET images at 24 h post injection of either NbHER2(WT) or NbHER2(D54FSY). Yellow arrow shows location of the NCI-N87 tumor.

FIG. 22C: 24 hour biodistribution of NbHER2(WT) labeled with ¹²⁴I and NbHER2(D54FSY) labeled with ¹²⁴I at different normal tissues and NCI N87 tumor. NbHER2(D54FSY) shows dramatic improvement of tumor retention over NbHER2(WT). Data are shown as mean±s.d (WT: n=2; D54FSY: n=3).

FIG. 23 shows binding affinity of NbHER2(WT) and NbHER2(D54FSY), ELISA measurements of NbHER2(WT) and NbHER2(D54FSY) with an IC50 of 2.4 nM and 7.6 nM, respectively (n=3).

FIG. 24 is a schematic showing development of a covalent ACE2 inhibitor via PERx to irreversibly inhibit SARS-CoV-2 infection.

FIG. 25 shows the FSY structure and its reaction with residue lysine, tyrosine, and histidine.

FIGS. 26A-26B show two different views of the ACE2-S protein binding interface, showing in stick the selected sites for FSY incorporation in ACE2 and the target residues in the S protein.

FIGS. 27A-27B show western blot analysis of FSY incorporation into the soluble ACE2 at the indicated sites in HEK293T cells. In FIG. 27A, supernatant of cell culture were analyzed. FIG. 27B shows western blot analysis of the ACE2-FSY proteins expressed and affinity purified from the Expi293F cells. An antibody specific for the His×6 tag appended at the C-terminus of ACE2 was used for detection.

FIGS. 28A-28B show covalent crosslinking of ACE2-FSY mutants with the spike protein of SARS-CoV-2 at 37° C. for 16 hours. In both cases, an antibody specific for the His×6 tag appended at the C-terminus of ACE2 was used for detection in these Western blots.

FIG. 29 shows Western blot analysis of ACE2-34FSY crosslinking with the S protein at the indicated time points.

FIG. 30 shows preparation of biotinylated SR4 using genetic code expansion and click chemistry.

FIGS. 31A-31E show generation of covalent nanobody to target the spike RBD via FSY incorporation. FIG. 31A: the principle of FSY reacts with a proximal nucleophile via SuFEx to develop covalent nanobody drugs. FIG. 31B: the crystal structure of nanobody H11-D4 complex with SARS-CoV-2 Spike RBD (PDB: 6YZ5). Sites selected for FSY incorporation in the nanobody and target residues of the spike RBD are shown in yellow and magenta stick, respectively. FIG. 31C: The crystal structure of nanobody MR17-K99Y in complex with the SARS-CoV-2 Spike RBD (PDB: 7CAN). FIG. 31D: the crystal structure nanobody of SR4 in complex with the SARS-CoV-2 Spike RBD (PDB: 7C8V). FIG. 31E: the ESI-MS spectrum of the intact nanobody SR4 (57FSY) confirming FSY incorporation.

FIGS. 32A-32E show nanobody(FSY) covalently cross-linked the spike RBD in vitro. FIG. 32A: cross-linking of purified H11-D4 and its mutants with the Spike RBD (molar ratio 1:5) at 37° C. overnight. Western blot analysis against mouse Fe tag appended at the C-terminus of the Spike RBD was used for detection. FIG. 32B: cross-linking of purified MR17-K99Y and its mutants with the Spike RBD (molar ratio 1:5) at 37° C. overnight. FIG. 32C: cross-linking of purified SR4 and its mutants with the Spike RBD (molar ratio 1:5) at 37° C. overnight. FIGS. 32D-32E: western blot analysis of SR4(54FSY) (5 μM) (D) or SR4(57FSY) (5 μM) (E) cross-linking with Spike RBD (0.5 μM) at indicated time points.

FIGS. 33A-33F show the covalent SR4(57FSY) inhibits RBD binding to cell surface ACE2 receptor and pseudoviral infection more effectively than the noncovalent WT SR4. FIG. 33A: assay scheme for nanobody inhibition of the Spike RBD binding to 293T-ACE2 cells. FIG. 33B: inhibition curve. Different concentrations of SR4 or SR4(57FSY) (2 μM, 1 μM, 0.2 M, 0.05 μM, 0.01 μM and 0.002 μM) inhibition of 10 nM Spike binding to 293T-ACE2 cells. The mean of fluorescence intensity (MFI) of spike binding to 293T-ACE2 cells was measured using mFc-FITC antibody by flow cytometry. n=3 biological replicates. Error bars represent s.e.m. FIGS. 33C-33D: scheme showing the principle of SR4 and SR4 (57FSY) inhibition of pseudovirus infection of 293T-ACE2 cells. FIGS. 33E-33F: inhibition of pseudovirus infection of 293T-ACE2 cells. Different concentrations of SR4 or SR4(57FSY) were incubated with pseudovirus, followed by dilution and 293T-ACE2 cell infection for 3 days. The percentage of GFP positive cells, the indicator for infection, was measured by flow cytometry. The normalized infection in y-axis was calculated using the following equation: (the percentage of positive GFP infected by pseudovirus with different concentrations of nanobodies incubation)/(the percentage of positive GFP infected by pseudovirus only)×100%. Error bars represent s.d., n=3 independent experiments.

FIGS. 34A-34L show nanobody SR4(57FSY) was able to covalently cross-link the RBDs of multiple mutant SARS-CoV-2 strains. FIGS. 34A-34F: biolayer interferometry (BLI) assay of the binding constant (K_{D}) between SR4 nanobody and wildtype or mutated spike protein. FIG. 34G-34L: The cross-linking rate measurement between SR4(57FSY) nanobody and wildtype or mutated spike.

FIGS. 35A-35C: are western blot analysis of the expression of H11-D4, MR17-K99Y or SR4 and their FSY mutants with or without FSY addition to the culture media.

FIGS. 36A-36C are SDS-PAGE analysis of purified H11-D4 and its FSY mutations (FIG. 36A), MR-17K99Y and its FSY mutants (FIG. 36B), and SR4 and its FSY mutants (FIG. 36C).

FIG. 37 shows the infectivity of SARS-CoV-2 pseudotyped lentivirus.

FIGS. 38A-38B show that the five mutated Spike proteins efficiently formed covalent adducts with SR4(57FSY). FIG. 38A provides results for spike proteins wild type (top panel), N501Y (middle panel), and F490L (bottom panel). FIG. 38B provides results for spike proteins E484K (top panel), N439K (middle panel), and K417N/E484K/N501Y (bottom panel).

FIGS. 39A-39B are Western blot analyses of crosslinking experiments with 7D12 FSY or mFSY nanobodies incubated with EGFR protein at 37° C. overnight.

FIGS. 40A-40B are Western blot analyses of crosslinking experiments with nanobody incubated with HER2 protein without heating samples (FIG. 40A) and with heating samples at 95° C. for 10 minutes (FIG. 40B).

FIGS. 41A-41B are Western blot analyses of crosslinking experiments with nanobodies incubated with HER2 and HER2 2RS15d protein using SKBR3 cells.

FIGS. 42A-42B show SDS-PAGE (FIG. 42A) and Western blot (FIG. 42B) analysis of crosslinking experiments with C21 nanobody incubated with CD16 protein at 37° C. for 16 hours.

FIG. 43 is a Western blot analysis of crosslinking experiments with NB13 nanobody with FSY incorporated at indicated sites incubated with and without PSMA protein.

FIGS. 44A-44B are Western blot analyses of crosslinking experiments with wildtype and mutant NB13 nanobodies incubated with PSMA+22rv1 cells.

FIGS. 45A-45B are Western blot analyses of crosslinking experiments with wildtype and mutant NB13 nanobodies incubated with PSMA+C4-2B wt (FIG. 45A) and PSMA-C4-2B k.o. cells (FIG. 45B).

FIGS. 46A-46B are Western blot analyses of crosslinking experiments with wildtype and mutant NB13 nanobodies incubated with PSMA+PC-3 pip (FIG. 46A) and PSMA-PC-3 flu cells (FIG. 46B).

FIG. 47 is a Western blot analysis of a crosslinking experiment with nanobodies with FSY or mFSY incorporated at indicated sites incubated with PSMA protein.

FIG. 48A-48K show Western blot (FIGS. 48A, 48C, 48E, 48G-48I) and Coomassie blue staining (FIGS. 48B, 48D, 48F, 48J, 48K) analyses of crosslinking experiments with Nb17B5 nanobodies incubated at 37° C. overnight with and without Her3 protein.

FIG. 49 shows 17B05-FSY mutant and Her3 protein crosslinking efficiency in vitro.

FIG. 50 are Western blot analyses of crosslinking experiments with MCF7 cells incubated with nanobodies and 100 ng/ml NRG.

FIG. 51 are Western blot analyses of crosslinking experiments with 22Rv1 cells incubated with nanobodies and different concentrations of NRG.

FIG. 52A-52D are Western blot analyses of crosslinking experiments with Nanobody 17B05 with mFSY incorporated incubated with Her3 protein.

FIG. 53 shows 17B05-mFSY mutant and Her3 crosslinking efficiency.

FIG. 54 is a Coomassie blue staining analysis of crosslinking experiments showing the kinetics of different nanobodies crosslinking with Her3 protein in vitro.

FIG. 55 is a Western blot analysis of crosslinking experiments with Affibody-Nanobody incubated with EGFR protein in PBS at 37° C. for 20 hours.

FIG. 56 is a Western blot analysis of crosslinking experiments with Affibody-Nanobody incubated with HER2 and/or EGFR proteins.

FIG. 57 is a Western blot analysis of crosslinking experiments with dimeric Affibody-Nanobody incubated with HER2 protein in PBS at 37° C. for 20 hours.

FIG. 58 is a Western blot analysis of crosslinking experiments with dimeric Affibody-Nanobody incubated with HER2 and/or EGFR protein in PBS at 37° C. for 20 hours.

FIG. 59 is a Western blot analysis of crosslinking experiments with bispecific Nanobody A-Nanobody B incubated with HER2 and/or EGFR protein in PBS at 37° C. for 20 hours.

FIGS. 60A-60I show mNb6(108FFY) neutralizes SARS-Cov-2 and certain variants with markedly increased potency over mNb6(WT). (FIG. 60A) mNb6(108FFY) showed 36-fold increase in potency than mNb6(WT) in inhibiting SARS-CoV-2 pseudovirus infection. (FIG. 60B) mNb6(108FFY) showed 41-fold increase in potency than mNb6(WT) in inhibiting authentic SARS-CoV-2 infection. (FIGS. 60C-60E) BLI of mNb6(WT) binding to the RBD of Alpha (C), Delta (D), and Beta (E) variant of SARS-Cov-2. Red traces show raw data, and black lines show kinetic fit. (FIG. 60F) Cross-linking of mNb6(108FFY) with the Spike RBD of SARS-CoV-2 variants in vitro. Incubation time was indicated. (FIG. 60G) mNb6(108FFY) showed 23-fold increase in potency than mNb6(WT) in inhibiting the Alpha variant pseudovirus infection. (FIG. 60H) mNb6(108FFY) showed 39-fold increase in potency than mNb6(WT) in inhibiting the Delta variant pseudovirus infection. For all pseudovirus and authentic SARS-Cov-2 inhibition experiments, n=3 independent repeats; error bars represent s.d.

FIGS. 61A-61D show covalent mNb6 dimer enhances viral neutralization over noncovalent WT mNb6 dimer. (FIG. 61A) Structure of dimer-WT and dimer-FFY. (FIG. 61B). Cross-linking of purified dimer-FFY with the Spike RBD in vitro. (FIG. 61C) Dimer-WT and dimer-FFY neutralizing pseudovirus infection of 293T-ACE2 cells. (FIG. 61D) Dimer-WT and dimer-FFY neutralizing authentic SARS-CoV-2 virus infection.

FIGS. 62A-62E show Western blot (FIGS. 62A and 62D) and SDS-PAGE (FIGS. 62B, 62C, and 62E) analysis of crosslinking experiment with A1 nanobody with FSY incorporated at indicated sites incubated with 0.5 μM mesothelin (MSLN) in PBS buffer at 37° C. for 12 hours.

FIGS. 63A-63B show SDS-PAGE analysis of crosslinking experiment with C6 nanobody with FSY incorporated at indicated sites incubated with 0.5 μM mesothelin (MSLN) in PBS buffer at 37° C. for 12 hours.

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The term “RNA-binding protein” refers to any protein capable of binding RNA. Examples of RNA-binding proteins include CRISPR proteins and RNA chaperones.

The term “CRISPR protein” or “CRISPR-associated protein” refers to any CRISPR protein in which catalytic sites for endonuclease activity are defective or lack activity. Exemplary CRISPR-associated proteins include dCas9, dCpf1, dCas12, dCas13, Cas-phi, a nuclease-deficient Cas9 variant, a nuclease-deficient Class II CRISPR endonuclease, and the like.

A “CRISPR-associated protein 9,” “Cas9,” “Csn1,” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

The terms “dCas9” or “dCas9 protein” as referred to herein is a Cas9 protein in which both catalytic sites for endonuclease activity are defective or lack activity. In aspects, the dCas9 protein has mutations at positions corresponding to D10A and H840A of S. pyogenes Cas9. In aspects, the dCas9 protein lacks endonuclease activity due to point mutations at both endonuclease catalytic sites (RuvC and HNH) of wild type Cas9. The point mutations can be D10A and H840A. In aspects, the dCas9 has substantially no detectable endonuclease (e.g., endodeoxyribonuclease) activity. In embodiments, the dCas9 from S. pyogenes. In embodiments, the dCas9 from S. aureus.

The terms “DNAse-dead Cpf1” or “ddCpf1” refer to mutated Acidaminococcus sp. Cpf1 (AsCpf1) resulting in the inactivation of Cpf1 DNAse activity. In aspects, ddCpf1 includes an E993A mutation in the RuvC domain of AsCpf1. In aspects, the ddCpf1 has substantially no detectable endonuclease (e.g., endodeoxyribonuclease) activity. In aspects, the ddCpf1 is from Lachnospiracea bacterium.

The term “dLbCpf1” refers to mutated Cpf1 from Lachnospiraceae bacterium ND2006 (LbCpf1) that lacks DNAse activity. In aspects, dLbCpf1 includes a D832A mutation. In aspects, the dLbCpf1 has substantially no detectable endonuclease (e.g., endodeoxyribo-nuclease) activity.

The term “dFnCpf1” refers to mutated Cpf1 from Francisella novicida U112 (FnCpf1) that lacks DNAse activity. In aspects, dFnCpf1 includes a D917A mutation. In aspects, the dFnCpf1 has substantially no detectable endonuclease (e.g., endodeoxyribo-nuclease) activity.

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In aspects, the Cpf1 protein is substantially identical to the protein identified by the UniProt reference number U2UMQ6 or a variant or homolog having substantial identity thereto. In aspects, the Cpf1 protein is identical to the protein identified by the UniProt reference number U2UMQ6.

The term “nuclease-deficient Cas9 variant” refers to a Cas9 protein having one or more mutations that increase its binding specificity to PAM compared to wild type Cas9 and further includes mutations that render the protein incapable of or having severely impaired endonuclease activity. Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). The binding specificity of nuclease-deficient Cas9 variants to PAM can be determined by any method known in the art. Descriptions and uses of known Cas9 variants may be found, for example, in Shmakov et al., Diversity and evolution of class 2 CRISPR-Cas systems. Nat. Rev. Microbiol. 15, 2017 and Cebrian-Serrano et al, CRISPR-Cas orthologues and variants: optimizing the repertoire, specificity and delivery of genome engineering tools. Mamm. Genome 7-8, 2017. Other Cas9 variants include Strep. pyogenes (Sp) Cas9, Staph. aureus (Sa) Cas9, SpCas9 VQR mutant (D1135V, R1335Q, T1337R), SpCas9 VRER mutant (D1135V, G121SR, R1335E, T1337R), SpCas9 (D1135E), eSpCas9 1.1 mutant (K848A-K1003A-R^{1060A}) SpCas9 HF1 (Q695A, Q926A, N497A, R661A) HypaCas9 (N692A, M694A, Q695A, H698A), and AsCpf1.

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The term “F(ab)′₂” is used interchangeably with “Fab dimer.” The F(ab)′2 may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). The term “Fab′ monomer” is used interchangeably with “Fab” and “or an antigen-binding fragment.” While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (e.g., McCafferty et al., Nature 348:552-554 (1990)).

Antibodies are large, complex proteins with an intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat et al, Sequences of Proteins of Immunological Interest, U.S. Department of Health and Human Services, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” and one “heavy” chain. The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

An “antibody variant” as provided herein refers to a polypeptide capable of binding to a receptor protein or an antigen and including one or more structural domains of an antibody or fragment thereof. Non-limiting examples of antibody variants include single-domain antibodies (nanobodies), affibodies (polypeptides smaller than monoclonal antibodies and capable of binding receptor proteins or antigens with high affinity and imitating monoclonal antibodies), antigen-binding fragments (Fab), Fab dimers (monospecific Fab₂, bispecific Fab₂), trispecific Fab₃, monovalent IgGs, single-chain variable fragments (scFv), bispecific diabodies, trispecific triabodies, scFv-Fc, minibodies, IgNAR, V-NAR, hcIgG, VhH, and peptibodies. A “peptibody” as provided herein refers to a peptide moiety attached (through a covalent or non-covalent linker) to the Fc domain of an antibody.

A “single-domain antibody” or “nanobody” refers to an antibody fragment having a single monomeric variable antibody domain. Like a whole antibody, it is able to bind selectively to a specific antigen. In embodiments, the single domain antibody is a human or humanized single-domain antibody.

A single-chain variable fragment (scFv) is typically a fusion protein of the variable regions of the heavy (VH) and light chains (VL) of immunoglobulins, connected with a short linker peptide of 10 to about 25 amino acids. The linker is usually rich in glycine for flexibility, as well as serine or threonine for solubility. The linker can either connect the N-terminus of the VH with the C-terminus of the VL, or vice versa.

Antibodies, e.g., recombinant, monoclonal, or polyclonal antibodies, can be prepared by techniques well known in the art (e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies can be adapted to produce antibodies to polypeptides. Also, transgenic mice, or other organisms such as other mammals, may be used to express humanized or human antibodies. Alternatively, phage display technology can be used to identify antibodies and heteromeric Fab fragments that specifically bind to selected antigens (e.g., McCafferty et al., Nature 348:552-554 (1990); Marks et al., Biotechnology 10:779-783 (1992)). Antibodies can also be made bispecific, i.e., able to recognize two different antigens (e.g., WO 93/08829, Traunecker et al., EMBO J. 10:3655-3659 (1991); Suresh et al., Methods in Enzymology 121:210 (1986)). Antibodies can also be heteroconjugates, e.g., two covalently joined antibodies, or immunotoxins (e.g., U.S. Pat. No. 4,676,980, WO 91/00360, WO 92/200373).

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (i) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (ii) the variable region, or a portion thereof, is altered, replaced or exchanged with a variable region having a different or altered antigen specificity. In embodiments, the antibodies described herein include humanized and/or chimeric monoclonal antibodies.

The phrase “specifically (or selectively) binds” to an antibody or a receptor protein or “specifically (or selectively) immunoreactive with” when referring to a protein refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (e.g., Harlow & Lane, Using Antibodies, A Laboratory Manual (1998) for a description of immunoassay formats and conditions that can be used to determine specific immunoreactivity).

“Receptor protein” or “membrane receptor” refers to a receptor (protein) that is embedded in the plasma membrane of a cell. In embodiments, the receptor protein is located in the extracellular domain of a cell, the transmembrane domain of a cell, or the intracellular domain of a cell. In embodiments, the receptor protein is a cell-surface receptor. In embodiments, the receptor protein is in the extracellular domain. In embodiments, the receptor protein is in the transmembrane domain. In embodiments, the receptor protein is an ion channel-linked receptor, an enzyme-linked receptor, or a G protein-coupled receptor. In embodiments, the receptor protein is a hormone receptor.

The term “peptidyl moiety” as used herein refers to a protein, protein fragment, or peptide that may form part of a biomolecule or a biomolecule conjugate. In aspects, the peptidyl moiety forms part of a biomolecule (e.g., protein). In aspects, the peptidyl moiety forms part of a biomolecule (e.g., protein) conjugate. The peptidyl moiety may also be substituted with additional chemical moieties (e.g., additional R substituents). In aspects, the peptidyl moiety forms part of an antibody or an antibody variant. In aspects, the peptidyl moiety forms part of a receptor protein. In aspects, a peptidyl moiety is a protein, protein fragment, or peptide that contains a monovalent radical of an amino acid.

The term “amino acid moiety” refers to a monovalent amino acid.

The term “carbohydrate moiety” as used herein refers to carbohydrates, for example, polyhydroxy aldehydes, ketones, alcohols, acids, their simple derivatives and their polymers having linkages of the acetal type, that may form part of a biomolecule or a biomolecule conjugate. In aspects, the carbohydrate moiety forms part of a biomolecule. In aspects, the carbohydrate moiety forms part of a biomolecule conjugate. The carbohydrate moiety may also be substituted with additional chemical moieties (e.g., additional R substituents).

The term “nucleic acid moiety” as used herein refers to nucleic acids, for example, DNA, and RNA, that may form part of a biomolecule or biomolecule conjugate. In aspects, the nucleic acid moiety forms part of a biomolecule. In aspects, the nucleic acid moiety forms part of a biomolecule conjugate. The nucleic acid moiety may also be substituted with additional chemical moieties (e.g., additional R substituents).

The term “lipid moiety” refers to a lipid or lipid fragment. The lipid may be substituted with additional chemical moieties. In embodiments, a lipid moiety is a monovalent radical of a lipid.

The term “RNA moiety” refers to a RNA, as described herein. In embodiments, an RNA moiety is a monovalent radical of RNA. In aspects, an RNA moiety is an RNA containing a monovalent radical of a nucleotide.

The term “RNA-binding protein moiety” refers to a protein, as described herein. In embodiments, an RNA-binding moiety is a monovalent radical of an RNA-binding protein, such as a monovalent radical of a CRISPR protein or a monovalent radical of a RNA chaperone.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Glycan Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In aspects, the amino acid side chain may be a non-natural amino acid side chain. In aspects, the amino acid side chain is H,

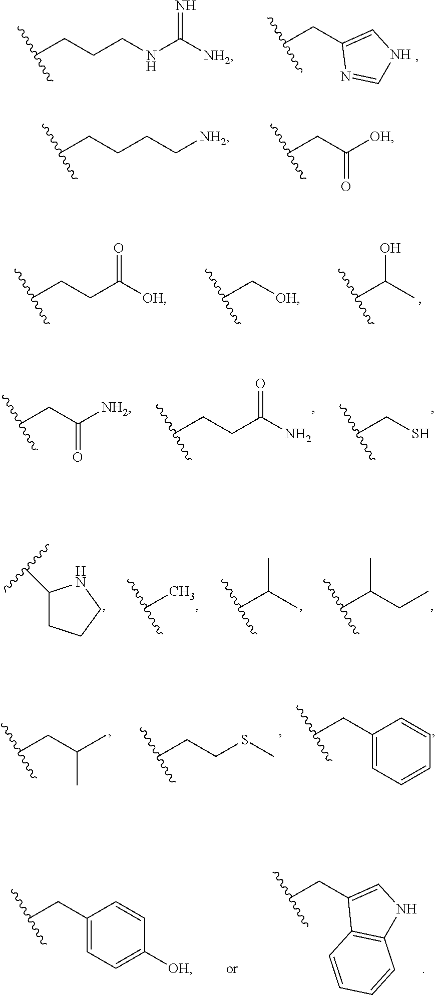

The term “non-natural amino acid side chain” or “unnatural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptane-carboxylic acid hydrochloride, cis-6-amino-3-cyclohexene-1-carboxylic acid hydrochloride, cis-2-amino-2-methylcyclohexanecarboxylic acid hydrochloride, cis-2-amino-2-methylcyclopentane-carboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholine acetic acid, Boc-pentafluoro-D-phenylalanine, Boc-pentafluoro-L-phenylalanine, Boc-Phe(2-Br)-OH, Boc-Phe(4-Br)-OH, Boc-D-Phe(4-Br)-OH, Boc-D-Phe(3-Cl)-OH, Boc-Phe(4-NH2)-OH, Boc-Phe(3-NO2)-OH, Boc-Phe(3,5-F2)-OH, 2-(4-Boc-piperazino)-2-(3,4-dimethoxy-phenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxy-phenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]-acetic acid purum, Boc-f-(2-quinolyl)-Ala-OH, N-Boc-1,2,3,6-tetrahydro-2-pyridinecarboxylic acid, Boc-j-(4-thiazolyl)-Ala-OH, Boc-j-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)-OH, Fmoc-Phe(4-Br)-OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

In embodiments, the unnatural amino acid is fluorosulfate-L-tyrosine or “FSY” having the following Formula (IE) or a stereoisomer thereof:

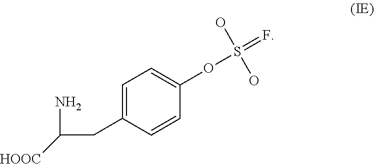

In embodiments, the unnatural amino acid side chain is the unnatural amino acid side chain of FSY, which is a moiety of Formula (JE-A) or a stereoisomer thereof:

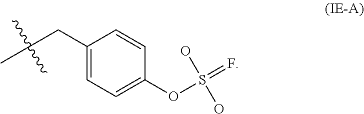

In embodiments, the unnatural amino acid is meta-fluorosulfate-L-tyrosine or “meta-FSY” or “mFSY” having the following Formula (IVA) or a stereoisomer thereof:

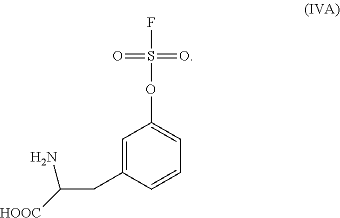 [SMILES: NC(Cc1cccc(OS(=O)(=O)F)c1)C(=O)O]

In embodiments, the unnatural amino acid side chain is the unnatural amino acid side chain of meta-FSY, which is a moiety of Formula (VA) or a stereoisomer thereof:

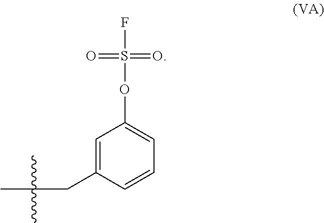 [SMILES: *Cc1cccc(OS(=O)(=O)F)c1]

In embodiments, the unnatural amino acid is “F-FSY” or “FFY” having the following Formula (VIID) or a stereoisomer thereof:

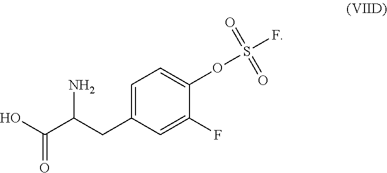 [SMILES: NC(Cc1ccc(OS(=O)(=O)F)c(F)c1)C(=O)O]

In embodiments, the unnatural amino acid side chain is the unnatural amino acid side chain of FFY, which is a moiety of Formula (VIIIC) or a stereoisomer thereof:

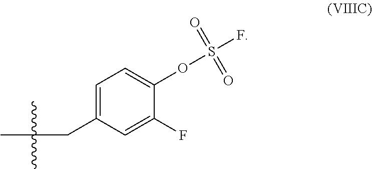 [SMILES: *Cc1ccc(OS(=O)(=O)F)c(F)c1]

In embodiments, the unnatural amino acids is meta-FSK, which is a compound of Formula (IVB) or a stereoisomer thereof:

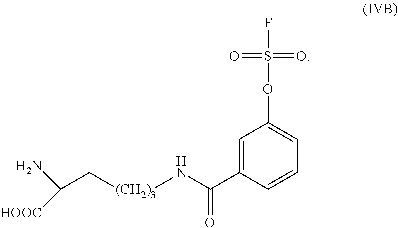

In embodiments, the unnatural amino acids is meta-FSK, wherein the unnatural amino acid side chain of meta-FSK is a moiety of Formula (VB) or a stereoisomer thereof:

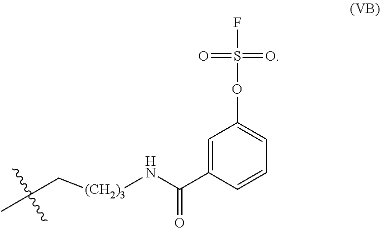

In embodiments, the unnatural amino acid is “fluorosulfonyloxybenzoyl-L-lysine” or “FSK” which is an unnatural amino acid having the following structure or a stereoisomer thereof:

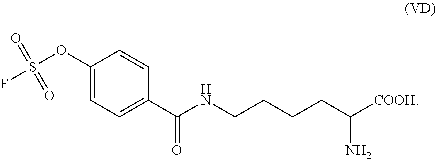 [SMILES: NC(CCCCNC(=O)c1ccc(OS(=O)(=O)F)cc1)C(=O)O]

In embodiments, the unnatural amino acids is FSK, wherein the unnatural amino acid side chain of FSK is a moiety having the following structure or a stereoisomer thereof:

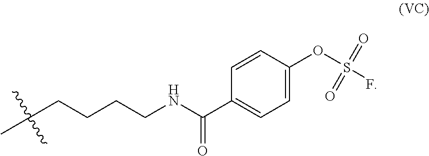 [SMILES: *CCCCNC(=O)c1ccc(OS(=O)(=O)F)cc1]

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (i) Alanine (A), Glycine (G); (ii) Aspartic acid (D), Glutamic acid (E); (iii) Asparagine (N), Glutamine (Q); (iv) Arginine (R), Lysine (K); (v) Isoleucine (I), Leucine (L), Methionine (M), Valine (V); (vi) Phenylalanine (F), Tyrosine (Y), Tryptophan (W); (vii) Serine (S), Threonine (T); and (viii) Cysteine (C), Methionine (M). (e.g., Creighton, Proteins (1984)).

The terms “polypeptide,” “peptide” and “protein” are used interchangeably herein to refer to a polymer of amino acid residues. The polymer of amino acids may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to position 133 (H133) of the catalytically inactive Cas13b protein from Prevotella sp. P5-125 (e.g., any one of SEQ ID NOS:48-48) when the selected residue occupies the same essential spatial or other structural relationship as position H133 of the catalytically inactive Cas13b protein from Prevotella sp. P5-125. In embodiments, where a selected protein is aligned for maximum homology with the catalytically inactive Cas13b protein from Prevotella sp. P5-125, the position in the aligned selected protein aligning with H133 is said to correspond to H133. Instead of a primary sequence alignment, a three dimensional structural alignment can also be used, e.g., where the structure of the selected protein is aligned for maximum correspondence with the catalytically inactive Cas13b protein from Prevotella sp. P5-125 and the overall structures compared. In this case, an amino acid that occupies the same essential position as H133 in the structural model is said to correspond to the H133 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, or at least 65%, 70%, 75%, 80%, 85%, 90%, 91%, 92%, 93%, 94%, 95%, 96%, 97%, 98%, 99% identity over a specified region, when compared and aligned for maximum correspondence over a comparison window or designated region) as measured using a BLAST or BLAST 2.0 sequence comparison algorithms with default parameters described below, or by manual alignment and visual inspection (e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “biomolecule” as used herein refers to large macromolecules such as, for example, proteins, lipids, and nucleic acids, as well as small molecules such as, for example, primary and secondary metabolites. In embodiments, the term biomolecule refers to a protein. In embodiments, the term biomolecule refers to a RNA-binding protein. In embodiments, the term biomolecule refers to RNA. In embodiments, the term biomolecule refers to a receptor protein.

The term “biomolecule moiety” as used herein refers to biomolecules, including large macromolecules such as, for example, proteins, lipids, and nucleic acids, as well as small molecules such as, for example, primary and secondary metabolites. Thus, in embodiments, the biomolecule moiety is a peptidyl moiety, a lipid moiety or a nucleic acid moiety. Biomolecule moieties may form part of a molecule (e.g., biomolecule). For example, biomolecule moieties may form part of a biomolecule conjugate, where the biomolecule conjugate includes two or more biomolecule moieties. In embodiments, the biomolecule conjugate includes two or more biomolecule moieties conjugated via a bioconjugate linker.

The term “pyrrolysyl-tRNA synthetase” refers to an enzyme (including homologs, isoforms, and functional fragments thereof) with pyrrolysyl-tRNA synthetase activity. Pyrrolysyl-tRNA synthetase is an aminoacyl-tRNA synthetase that catalyzes the reaction necessary to attach α-amino acid pyrrolysine to the cognate tRNA (tRNA^{pyl}), thereby allowing incorporation of pyrrolysine during proteinogenesis at amber stop codons (i.e., UAG). The term includes any recombinant or naturally-occurring form of pyrrolysyl-tRNA synthetase or variants, homologs, or isoforms thereof that maintain pyrrolysyl-tRNA synthetase activity (e.g. within at least 30%, 40%, 50%, 60%, 70%, 80%, 90%, 95%, or 100% activity compared to wild-type pyrrolysyl-tRNA synthetase). In embodiments, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring pyrrolysyl-tRNA synthetase. In embodiments, the mutant pyrrolysyl-tRNA synthetase catalyzes the attachment of the compound of Formula (I) and embodiments thereof to a tRNA^{pyl}. In embodiments, the mutant pyrrolysyl-tRNA synthetase catalyzes the attachment of the compound of Formula (IV) and embodiments thereof to a tRNA^{pyl}. In embodiments, the mutant pyrrolysyl-tRNA synthetase catalyzes the attachment of the compound of Formula (VII) and embodiments thereof to a tRNA^{pyl}. In embodiments, the pyrrolysyl-tRNA synthetase comprises the amino acid sequence set forth as SEQ ID NO:49, SEQ ID NO:56, SEQ ID NO:57, or SEQ ID NO:58.

The term “mutant pyrrolysyl-tRNA synthetase” or “mutant PyIRS” refers to any pyrrolysyl-tRNA synthetase that has a different amino acid sequence from wild-type amino acid sequence.

The terms “tRNA^{Pyl}” and “rTNA^{Pyl}_{CUA}” and “tRNA_{CUA}^{Pyl}” (i.e., tRNA(superscript Pyl)(subscript CUA)) are used interchangeably and all refer to a single-stranded RNA molecule containing about 70 to 90 nucleotides which fold via intrastrand base pairing to form a characteristic cloverleaf structure that carries a specific amino acid (e.g., compound of Formula (I) or embodiments thereof; compound of Formula (IV) or embodiments thereof; compound of Formula (VII) or embodiments thereof) and matches it to its corresponding codon (i.e., a complementary to the anticodon of the tRNA) on an mRNA during protein synthesis. In tRNA^{Pyl}, the anticodon is CUA. Anticodon CUA is complementary to amber stop codon UAG. In embodiments, the tRNA^{Pyl} comprises an anticodon. In embodiments, the anticodon is CUA, TTA, or TCA. In embodiments, the tRNA^{Pyl} comprises an anticodon, wherein the anticodon comprises at least one non-cannonical base. The abbreviation “Pyl” of tRNA^{Pyl} stands for pyrrolysine and the “CUA” of tRNA^{Pyl} refers to its anticodon CUA. In embodiments, tRNA^{Pyl} is attached to the compound of Formula (I) or embodiments thereof. In embodiments, tRNA^{Pyl} is attached to the compound of Formula (IV) or embodiments thereof. In embodiments, tRNA^{Pyl} is attached to the compound of Formula (VII) or embodiments thereof.

The term “substrate-binding site” as used herein refers to residues located in the enzyme active site that form temporary bonds or interactions with the substrate. In embodiments, the substrate-binding site of pyrrolysyl-tRNA synthetase refers to residues located in the active site of pyrrolysyl-tRNA synthetase that form temporary bonds or interactions with the amino acid substrate.

The term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. The terms “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Some viral vectors are capable of targeting a particular cells type either specifically or non-specifically. Exemplary vectors that can be used include, but are not limited to, pEvol vector, pMP vector, pET vector, pTak vector, pBad vector.

The term “complex” refers to a composition that includes two or more components, where the components bind together to make a functional unit. In embodiments, a complex described herein include a mutant pyrrolysyl-tRNA synthetase described herein and an amino acid substrate (e.g., the compound of Formula (I) or embodiments thereof; the compound of Formula (IV) or embodiments thereof; the compound of Formula (VII) or embodiments thereof). In embodiments, a complex described herein includes a mutant pyrrolysyl-tRNA synthetase described herein and a tRNA (e.g., tRNA^{Py}). In embodiments, a complex described herein includes a mutant pyrrolysyl-tRNA synthetase described herein, an amino acid substrate (e.g., FSY, mFSY, FFSY) and a tRNA (e.g., tRNA^{Py}). In embodiments, a complex described herein includes at least two components selected from the group consisting of a mutant pyrrolysyl-tRNA synthetase described herein, an amino acid substrate (e.g., the compound of Formula (I) or embodiments thereof), a polypeptide containing the compound of Formula (I) or embodiments thereof, and a tRNA (e.g., tRNA^{Py}). In embodiments, a complex described herein includes at least two components selected from the group consisting of a mutant pyrrolysyl-tRNA synthetase described herein, an amino acid substrate (e.g., the compound of Formula (IV) or embodiments thereof), a polypeptide containing the compound of Formula (IV) or embodiments thereof, and a tRNA (e.g., tRNA^{Py}). In embodiments, a complex described herein includes at least two components selected from the group consisting of a mutant pyrrolysyl-tRNA synthetase described herein, an amino acid substrate (e.g., the compound of Formula (VII) or embodiments thereof), a polypeptide containing the compound of Formula (VII) or embodiments thereof, and a tRNA (e.g., tRNA^{Py}).

The term “RNA-binding protein/RNA complex” refers to a composition that includes one RNA-binding protein and one RNA, where the RNA-binding protein and RNA are proximal to each other but not bound together; the RNA-binding protein and RNA are covalently bound together; or the RNA-binding protein and RNA are ionically bound together. In embodiments, the RNA-binding protein and RNA are proximal to each other but not bound together. In embodiments, the RNA-binding protein and RNA are covalently bonded together. In embodiments, the RNA-binding protein and RNA are ionically bonded together. In embodiments, the RNA-binding protein and RNA are covalently and ionically bonded together. In embodiments, the chemical reaction forming the RNA-binding protein/RNA complex is a SuFEx reaction.

The term “protein/protein complex” refers to a composition that includes one protein-binding protein (e.g., comprising an unnatural amino acid as described herein) and one protein, where the protein-binding protein and protein are proximal to each other but not bound together; the protein-binding protein and protein are covalently bound together; or the protein-binding protein and protein are ionically bound together. In embodiments, the protein-binding protein and protein are proximal to each other but not bound together. In embodiments, the protein-binding protein and protein are covalently bonded together. In embodiments, the protein-binding protein and protein are ionically bonded together. In embodiments, the protein-binding protein and protein are covalently and ionically bonded together. In embodiments, the chemical reaction forming the protein/protein complex is a SuFEx reaction.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Contacting” is used in accordance with its plain ordinary meaning and refers to the process of allowing at least two distinct species (e.g. chemical compounds including amino acids, proteins, peptides, biomolecules, or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be biomolecule moieties as described herein. In some embodiments, contacting includes allowing two proteins or a protein and a glycan as described herein to interact.

A “detectable agent” or “detectable moiety” is a composition detectable by appropriate means such as spectroscopic, photochemical, biochemical, immunochemical, chemical, magnetic resonance imaging, or other physical means. In embodiments, the proteins described herein are bonded to a detectable agent. In embodiments, the fusion proteins described herein are bonded to a detectable agent. In embodiments, an antibody or antibody variant is bonded to a detectable agent. In embodiments, a nanobody is bonded to a detectable agent. In embodiments, the bond is noncovalent or covalent. In embodiments, the bond is covalent. In embodiments, the protein is covalently bonded to a detectable agent. In embodiments, the fusion protein is covalently bonded to a detectable agent. In embodiments, the antibody or antibody variant is covalently bonded to a detectable agent. In embodiments, a nanobody is covalently bonded to a detectable agent. In embodiments when the protein or fusion protein is covalently bonded to a detectable agent, the covalent bond is between the detectable agent and a naturally-occurring amino acid in the protein or fusion protein. In embodiments when the nanobody is covalently bonded to a detectable agent, the covalent bond is between the detectable agent and a naturally-occurring amino acid in the nanobody. Methods for covalently bonding detectable agents to proteins are well-known in the art. Detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gases, perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition. In embodiments, paramagnetic ions that may be used as imaging agents in accordance with the embodiments of the disclosure include, e.g., ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

A “radioisotope” that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶D, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. In embodiments, the proteins described herein are bonded to a radioisotope. In embodiments, the fusion proteins described herein are bonded to a radioisotope. In embodiments, an antibody or antibody variant is bonded to a radioisotope. In embodiments, a nanobody is bonded to a radioisotope. In embodiments, the bond is noncovalent or covalent. In embodiments, the bond is covalent. In embodiments, the protein is covalently bonded to a radioisotope. In embodiments, the fusion protein is covalently bonded to a radioisotope. In embodiments, the antibody or antibody variant is covalently bonded to a radioisotope. In embodiments, a nanobody is covalently bonded to a radioisotope. In embodiments when the protein or fusion protein is covalently bonded to a radioisotope, the covalent bond is between the radioisotope and a naturally-occurring amino acid in the protein or fusion protein. In embodiments when the nanobody is covalently bonded to a radioisotope, the covalent bond is between the radioisotope and a naturally-occurring amino acid in the nanobody. Methods for covalently bonding radioisotopes to proteins are well-known in the art. In embodiments, the radioisotope is ¹²³I, ¹²⁴I, ¹²⁵I, or ¹³¹I. In embodiments, the radioisotope is ¹²³I. In embodiments, the radioisotope is ¹²⁴I. In embodiments, the radioisotope is ¹²⁵I. In embodiments, the radioisotope is ¹³¹I. In embodiments, the radioisotope is a positron-emitting radioisotope. In embodiments, the positron-emitting radioisotope is ¹¹C, ¹³N, ¹⁵O, ¹⁸F, ⁶⁴Cu, ⁶⁸Ga, ⁷⁸Br, ⁸²Rb, ⁸⁶Y, ⁸⁹Zr, ⁹⁰Y, ²²Na, ²⁶Al, ⁴⁰K, ⁸³Sr, or ¹²⁴I. In embodiments, the positron-emitting radioisotope is ¹¹C. In embodiments, the positron-emitting radioisotope is ¹³N. In embodiments, the positron-emitting radioisotope is ¹⁵O. In embodiments, the positron-emitting radioisotope is ¹⁸F. In embodiments, the positron-emitting radioisotope is ⁶⁴Cu. In embodiments, the positron-emitting radioisotope is ¹⁶⁸Ga. In embodiments, the positron-emitting radioisotope is ⁷⁸Br. In embodiments, the positron-emitting radioisotope is ⁸²Rb. In embodiments, the positron-emitting radioisotope is ⁸⁶Y. In embodiments, the positron-emitting radioisotope is ⁸⁹Zr. In embodiments, the positron-emitting radioisotope is ⁹⁰Y. In embodiments, the positron-emitting radioisotope is ²²Na. In embodiments, the positron-emitting radioisotope is ²⁶Al. In embodiments, the positron-emitting radioisotope is ⁴⁰K. In embodiments, the positron-emitting radioisotope is ⁸³Sr. In embodiments, the positron-emitting radioisotope is ¹²⁴I. In embodiments, the radioisotope is an alpha-emitting radioisotope. In embodiments, the alpha-emitting radioisotope is ²¹¹At, ²²⁷Th, ²²⁵Ac, ²²³Ra, ²¹³Bi, or ²¹²Bi. In embodiments, the alpha-emitting radioisotope is ²¹¹At. In embodiments, the alpha-emitting radioisotope is ²²⁷Th. In embodiments, the alpha-emitting radioisotope is ²²⁵Ac. In embodiments, the alpha-emitting radioisotope is ²²³Ra. In embodiments, the alpha-emitting radioisotope is ²¹³Bi. In embodiments, the alpha-emitting radioisotope is ²¹²Bi.

The term “therapeutic agent” refers to any agent useful in treating and/or preventing a disease. “Therapeutic agent” includes, without limitation, small molecule drugs, proteins, nucleic acids (e.g., DNA, RNA), and the like. “Small-molecule drugs” refers to chemical compounds with low molecular weight that are capable of treating and/or preventing diseases. In embodiments, the proteins described herein are bonded to a therapeutic agent. In embodiments, the fusion proteins described herein are bonded to a therapeutic agent. In embodiments, an antibody or antibody variant is bonded to a therapeutic agent. In embodiments, a nanobody is bonded to a therapeutic agent. In embodiments, the bond is noncovalent or covalent. In embodiments, the bond is covalent. In embodiments, the protein is covalently bonded to a therapeutic agent. In embodiments, the fusion protein is covalently bonded to a therapeutic agent. In embodiments, the antibody or antibody variant is covalently bonded to a therapeutic agent. In embodiments, a nanobody is covalently bonded to a therapeutic agent. In embodiments when the protein or fusion protein is covalently bonded to a therapeutic agent, the covalent bond is between the therapeutic agent and a naturally-occurring amino acid in the protein or fusion protein. In embodiments when the nanobody is covalently bonded to a therapeutic agent, the covalent bond is between the therapeutic agent and a naturally-occurring amino acid in the nanobody. Methods for covalently bonding therapeutic agents to proteins are well-known in the art.

The term “sulfur-fluoride exchange reaction” or “SuFEx” refers to a type of click chemistry as described in detail by, e.g., Dong et al, Angewandte Chemie, 53(36):9340-9448 (2014); and Wang et al, J. Am. Chem. Soc., 140(15):4995-4999 (2018). The term “proximally-enabled” SuFEx refers to the sulfur-fluoride exchange reaction occurring when the reactive species are proximal to each other, i.e., spatially close enough for the SuFEx reaction to occur. The proximity may occur within a single biomolecule (e.g., protein) or between two different biomolecules (e.g., protein and RNA). The skilled artisan could readily determine whether the reactive species are sufficiently proximal for the reaction to occur, e.g., sulfur-fluoride exchange reaction between the compound of Formula (I) and RNA (e.g., a hydroxyl group on RNA). The skilled artisan could readily determine whether the reactive species are sufficiently proximal for the reaction to occur, e.g., sulfur-fluoride exchange reaction between the compound of Formula (IV) and a peptidyl moiety (e.g., having a tyrosine, lysine, or histidine), a nucleic acid moiety, or a carbohydrate moiety; or for example a sulfur-fluoride exchange reaction between the compound of Formula (I) and a nucleic acid moiety; or for example a sulfur-fluoride exchange reaction between the compound of Formula (VII) and a peptidyl moiety (e.g., having a tyrosine, lysine, or histidine), a nucleic acid moiety, or a carbohydrate moiety.

In embodiments, “proximal” means that two compounds (e.g., biomolecules, proteins, peptides, amino acids, glycans) are adjacent (e.g., but not covalently bonded together). In embodiments, “proximal” means up to about 25 angstroms. In embodiments, “proximal” means up to about 20 angstroms. In embodiments, “proximal” means up to about 15 angstroms. In embodiments, “proximal” means up to about 10 angstroms. In embodiments, “proximal” means from about 1 angstrom to about 25 angstroms. In embodiments, “proximal” means from about 1 angstrom to about 20 angstroms. In embodiments, “proximal” means from about 1 angstrom to about 15 angstroms. In embodiments, “proximal” means from about 1 angstrom to about 12 angstroms. In embodiments, “proximal” means from about 1 angstrom to about 10 angstroms. In embodiments, “proximal” means from about 1 angstrom to about 8 angstroms. In embodiments, “proximal” means from about 1 angstrom to about 6 angstroms. In embodiments, “proximal” means from about 1 angstrom to about 5 angstroms. In embodiments, “proximal” means from about 1 angstroms to about 4 angstroms.

The term “intermolecular linker” refers to a linking group between two biomolecules. For example, when the compounds of Formula (VI) or (IX) (or embodiments thereof) are an intermolecular linker, then the peptidyl moiety of R⁴ is a first protein and the peptidyl moiety of R⁵ is a second protein, such that the first protein and the second protein are covalently bonded. In aspects, the first protein and the second protein can have the same sequence, e.g., providing an intermolecular linker between two different proteins having the same amino acid sequence. In aspects, the first protein and the second protein are different proteins, e.g., providing an intermolecular linker between two different proteins, such as a Fab and a receptor protein.

The term “intramolecular linker” refers to a linking group within a biomolecule. For example, when the compounds of Formula (VI) or (IX) (or embodiments thereof) are an intramolecular linker, then the peptidyl moiety of R⁴ and the peptidyl moiety of R⁵ are in the same protein. A compound having an intramolecular linker may also be referred to as an intramolecularly conjugated biomolecule conjugate or an intramolecularly conjugated biomolecule protein.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified by, e.g., —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) may be placed at any interior position of the heteroalkyl group or at the position at which the alkyl group is attached to the remainder of the molecule. Heteroalkyl is an uncyclized chain. Examples include, but are not limited to: —CH₂—CH₂O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)3, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)3. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C1-C4)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” or “-” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker).

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In embodiments, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl, heteroalkyl, cycloalkyl, heterocycloalkyl, aryl, heteroaryl, substituted with at least one substituent selected from: (i) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl, heteroalkyl, cycloalkyl, heterocycloalkyl, aryl, heteroaryl, substituted with at least one substituent selected from: (a) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl, heteroalkyl, cycloalkyl, heterocycloalkyl, aryl, heteroaryl, substituted with at least one substituent selected from: oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNI₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. As used herein, the term “isomers” refers to compounds having the same number and kind of atoms, and hence the same molecular weight, but differing in respect to the structural arrangement or configuration of the atoms. The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers (stereoisomers) as well as enantiomeric and diastereomeric mixtures of the present compounds are within the scope of the disclosure.

The compounds described herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds described herein, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R³ substituents are present, each R³ substituent may be distinguished as R^{3A}, R^{3B}, wherein each of R^{3A}, R^{3B}, is defined within the scope of the definition of R³ and optionally differently.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “bond” or “bonded” refers to direct bonds, such as covalent bonds (e.g., direct or a linking group), or indirect bonds, such as non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions, and the like).

The terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al, Modification of Proteins, Advances in Chemistry Series, Vol. 198, American Chemical Society, Washington, D.C., 1982. In embodiments, the first bioconjugate reactive group (e.g., unnatural amino acid side chain) is covalently attached to the second bioconjugate reactive group (e.g., a hydroxyl group).

The term “electron-withdrawing group” refers to a chemical moiety or substituent that removes electron density from a conjugated pi-electron system, thereby making the pi electron system less electrophilic.

The term “electron-donating group” refers to a chemical moiety or substituent that can donate electron density into a conjugated pi-electron system, thereby making the pi electron system more nucleophilic.

“Viral spike (S) protein” refers to the viral spike (S) protein of a coronavirus which binds to the cellular angiotensin-converting enzyme 2 (ACE2) receptor protein, and includes any of the recombinant or naturally-occurring forms of the viral spike (S) protein or variants or homologs thereof that maintain viral spike (S) protein activity (e.g., within at least 50%, 80%, 90%, 95%, 96%, 97%, 98%, 99% or 100% activity compared to viral spike (S) protein). In some aspects, the variants or homologs have at least 75%, 76%, 77%, 78%, 79%, 80%, 81%, 82%, 83%, 84% 85%, 86%, 87%, 88%, 89%, 90%, 91%, 92%, 93%, 94%, 95%, 96%, 97%, 98%, 99% or 100% amino acid sequence identity across the whole sequence or a portion of the sequence (e.g. a 50, 100, 150, 200, 250, 300, 350, 400, 450, 500 continuous amino acid portion) compared to a naturally occurring viral spike (S) protein. In aspects, the viral spike (S) protein is substantially identical to the protein identified as SEQ ID NO:5 or a variant or homolog having substantial identity thereto. In aspects, the viral spike (S) protein is a conservatively modified variant of the protein identified as SEQ ID NO:5. In aspects, the viral spike (S) protein has one or more mutations. In aspects, the viral spike (S) protein has one or more mutations at positions corresponding to K417, N439, E484, F490, and N501.

“ACE2 receptor protein” and “ACE2 protein” as referred to herein includes any of the recombinant or naturally-occurring forms of the angiotensin-converting enzyme 2 (ACE2) protein or variants or homologs thereof that maintain ACE2 activity (e.g. within at least 50%, 80%, 90%, 95%, 96%, 97%, 98%, 99% or 100% activity compared to ACE2). In some aspects, the variants or homologs have at least 80%, 85%, 90%, 95%, 96%, 97%, 98%, 99% or 100% amino acid sequence identity across the whole sequence or a portion of the sequence (e.g. a 50, 100, 150, 200, 250, 300, 350, 400, 450, 500 continuous amino acid portion) compared to a naturally occurring ACE2 protein. In aspects, the ACE2 protein is substantially identical to the protein identified as SEQ ID NO:1 or a variant or homolog having substantial identity thereto. In aspects, the ACE2 protein is substantially identical to the portion of the protein spanning amino acid residues 19 to 615 in SEQ ID NO:1 or a variant or homolog having substantial identity thereto.

“SARS” refers to severe acute respiratory syndrome.

“SARS-CoV” refers to severe acute respiratory syndrome-associated coronavirus.

“SARS-CoV-1” refers to severe acute respiratory syndrome-associated coronavirus 1.

“SARS-CoV-2” refers to severe acute respiratory syndrome-associated coronavirus 2.

“COVID-19” refers to the disease caused by SARS-CoV-2. COVID-19 has an incubation period of 2-14 days, and symptoms include, e.g., fever, tiredness, cough, and shortness of breath (e.g., difficulty breathing).

“MERS-CoV” refers to Middle Eastern respiratory syndrome-associated coronavirus. See, e.g., Chung et al, Genetic Characterization of Middle East Respiratory Syndrome Coronavirus, South Korea, 2018. Emerging Infectious Diseases, 25(5):958-962 (2019).

“Middle Eastern respiratory syndrome” or “MERS” refers to the disease caused by MERS-coronavirus.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g., a single-domain antibody or a recombinant protein as described herein, i.e., comprising an unnatural amino acid side chain that is capable of binding to an amino acid residue on a different protein) that is able to measurably bind to a target (e.g., a viral spike (S) protein of SARS-CoV). In aspects, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

### Compounds

Provided herein are proteins comprising unnatural amino acid side chains and biomolecules formed through the interaction of the unnatural amino acids with naturally occurring amino acids or nucleotides. The compounds of Formula (I), Formula (IV), and Formula (VII), i.e., bioreactive unnatural amino acids, facilitate formation of chemically reactive amino acids with proximal target amino acid residues by undergoing a click chemistry reaction (e.g., sulfur-fluoride exchange reaction (SuFEx)). For example, the compounds of Formula (I), Formula (IV), or Formula (VII) may be inserted into or replace an amino acid in a naturally occurring protein, thereby endowing the protein with the ability to form a chemically reactive amino acid with proximally positioned target functional groups (e.g., a hydroxyl group in RNA) or amino acid residues (e.g., serine, threonine, tyrosine) with other proteins. The compound of Formula (I), Formula (IV), and Formula (VII) may be used to facilitate the formation of chemically reactive amino acids in proteins in both in vitro and in vivo conditions. As such, the bioreactive unnatural amino acids of Formula (I), Formula (IV), and Formula (VII) are useful for forming chemically reactive amino acid residues that can be further chemically modified.

The compounds of Formula (I), Formula (IV), and Formula (VII) have shown excellent chemical functionality (i.e., superior properties) compared to previously described bioreactive unnatural amino acids. For example, the compounds of Formula (I), Formula (IV), and Formula (VII) are stable, nontoxic and nonreactive inside cells, yet when placed in proximity to target amino acid residues (e.g., serine, threonine, tyrosine) or reactive moieties (e.g., a hydroxyl group in RNA) they becomes reactive under cellular conditions. The compounds of Formula (I), Formula (IV), and Formula (VII) are able to react with target amino acid residues (e.g., serine, threonine, tyrosine) or other reactive moieties (e.g., a hydroxyl group in RNA) with great selectivity via proximity-enabled SuFEx reaction within and between proteins and RNA under physiological conditions.

Provided herein are compounds of Formula (I):

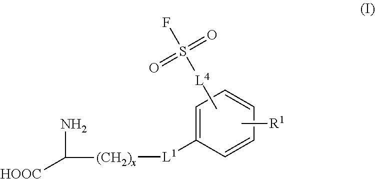

or the stereoisomer thereof of Formula (I-1):

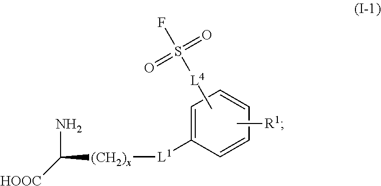

wherein L⁴ is a bond or —O—; and R¹, L¹, and x are as defined herein. In embodiments, L⁴ is a bond. In embodiments, L⁴ is —O—. In embodiments, R¹ is an electron-donating group or an electron-withdrawing group. In embodiments, when L⁴ is a bond then R¹ is an electron-donating group. In embodiments, when L⁴ is —O— then R¹ is an electron-withdrawing group. In embodiments -L⁴S(═O)₂F is para, meta, or ortho to the carbon atom linked to L¹. In embodiments -L⁴S(═O)₂F is para to the carbon atom linked to L¹. In embodiments -L⁴S(═O)₂F is meta to the carbon atom linked to L¹. In embodiments -L⁴S(═O)₂F is ortho to the carbon atom linked to L¹. In embodiments, R¹ is ortho, meta, or para to -L⁴S(═O)₂F. In embodiments, R¹ is ortho to -L⁴S(═O)₂F. In embodiments, R¹ is meta to -L⁴S(═O)₂F. In embodiments, R¹ is para to -L⁴S(═O)₂F.

In embodiments, the compound of Formula (I) is a compound of Formula (IA):

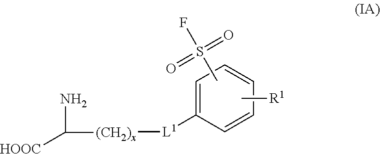

or the stereoisomer thereof of Formula (IA-1):

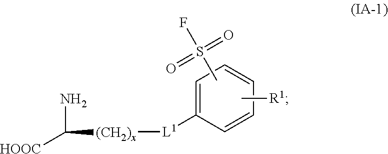

wherein R¹, L¹, and x are as defined herein. In embodiments, R¹ is an electron-donating group.

In embodiments, the compound of Formula (I) is a compound of Formula (IB):

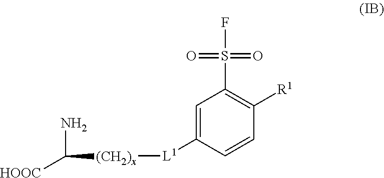

or the stereoisomer thereof of Formula (IB-1):

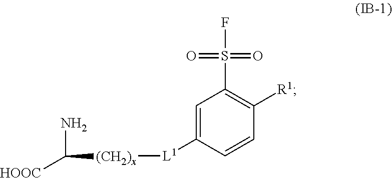

wherein R¹, L¹, and x are as defined herein. In embodiments, R¹ is an electron-donating group.

In embodiments, the compound of Formula (I) is a compound of Formula (IC):

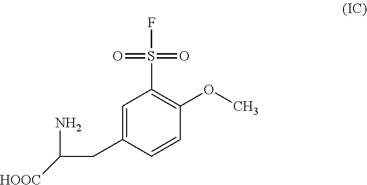 [SMILES: COc1ccc(CC(N)C(=O)O)cc1S(=O)(=O)F]

or the stereoisomer thereof of Formula (IC-1):

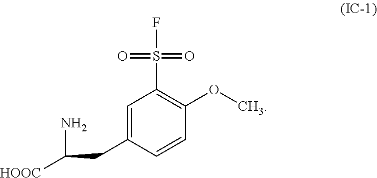 [SMILES: COc1ccc(C[C@H](N)C(=O)O)cc1S(=O)(=O)F]

In embodiments, the compound of Formula (IC) is referred to as SFY.

In embodiments, the compound of Formula (I) is a compound of Formula (ID):

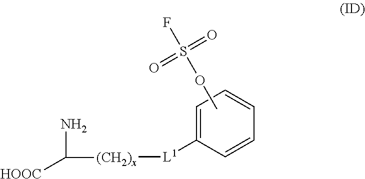

or the stereoisomer thereof of Formula (ID-1):

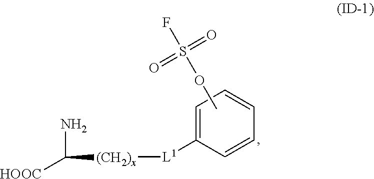

wherein L¹ and x are as defined herein.

In embodiments, the compound of Formula (I) is a compound of Formula (IE):

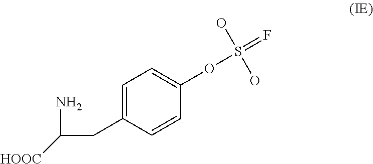

or the stereoisomer thereof of Formula (IE-1):

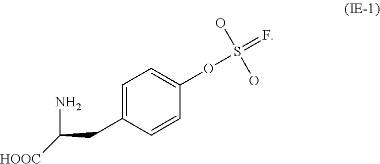

In embodiments, the compound of Formula (IE) is referred to as FSY.

Provided herein are compounds of Formula (IV):

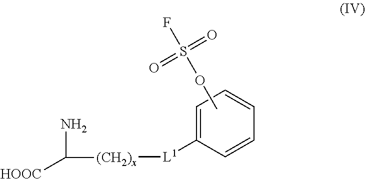

or the stereoisomer thereof of Formula (IV-1):

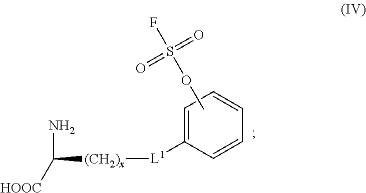

wherein —OS(═O)₂F is meta or ortho to the carbon atom linked to L¹; x is an integer from 1 to 8; and L¹ is a bond, substituted or unsubstituted alkylene, or substituted or unsubstituted heteroalkylene. In embodiments, —OS(═O)₂F is ortho to the carbon atom linked to L¹. In embodiments, —OS(═O)₂F is meta to the carbon atom linked to L¹.

In embodiments, the compound of Formula (IV) is a compound of Formula (IVA):

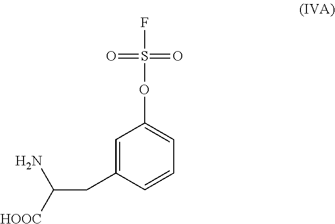 [SMILES: NC(Cc1cccc(OS(=O)(=O)F)c1)C(=O)O]

or the stereoisomer thereof of Formula (IVA-1):

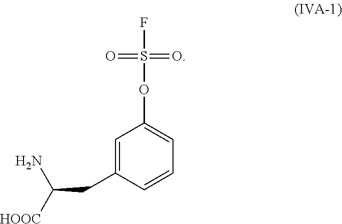 [SMILES: N[C@@H](Cc1cccc(OS(=O)(=O)F)c1)C(=O)O]

The compound of Formula (IVA) is optionally referred to as meta-FSY, metaFSY, or mFSY.

In embodiments, the compound of Formula (IV) is a compound of Formula (IVB):

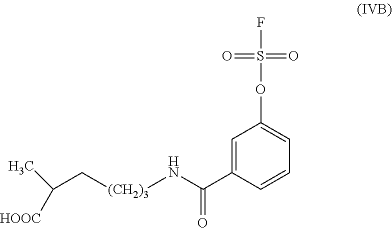

or the stereoisomer thereof of Formula (IVB-1):

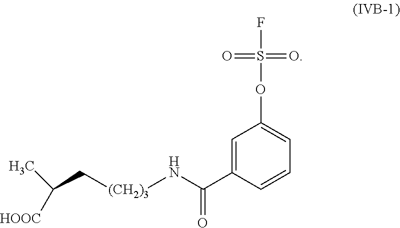

The compound of Formula (IVB) is optionally referred to as meta-FSK, metaFSK, or mFSK.

Provided herein are compounds of Formula (VII):

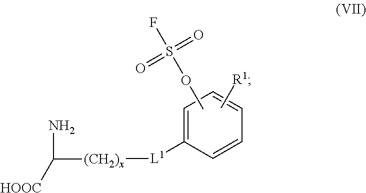

wherein R¹, L¹, and x are as defined herein. In embodiments, R¹ is an electron-withdrawing group. In embodiments, —OS(═O)₂F is ortho, meta, or para to the carbon atom linked to L¹. In embodiments, —OS(═O)₂F is ortho to the carbon atom linked to L¹. In embodiments, —OS(═O)₂F is meta to the carbon atom linked to L¹. In embodiments, —OS(═O)₂F is para to the carbon atom linked to L¹. In embodiments, R¹ is ortho, meta, or para to —OS(═O)₂F. In embodiments, R¹ is ortho to —OS(═O)₂F. In embodiments, R¹ is meta to —OS(═O)₂F. In embodiments, R¹ is para to —OS(═O)₂F.

In embodiments, the compound of Formula (VII) is a compound of Formula (VIIA):

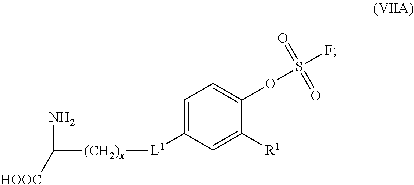

wherein R¹, L¹, and x are as defined herein. In embodiments, R¹ is an electron-withdrawing group.

In embodiments, the compound of Formula (VII) is a compound of Formula (VIIB):

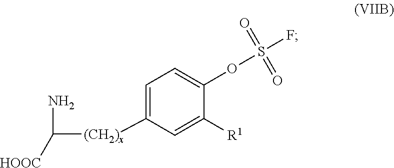

wherein R¹, L¹, and x are as defined herein. In embodiments, R¹ is an electron-withdrawing group.

In embodiments, the compound of Formula (VII) is a compound of Formula (VIIC):

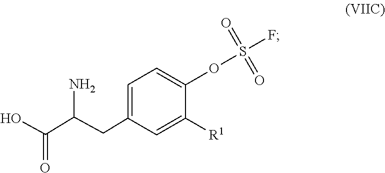

wherein R¹ is as defined herein. In embodiments, R¹ is an electron-withdrawing group.

In embodiments, the compound of Formula (VII) is referred to as “F-FSY” or “FFY” and is represented by the compound of Formula (VIID):

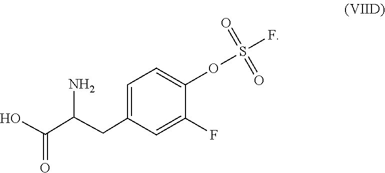 [SMILES: NC(Cc1ccc(OS(=O)(=O)F)c(F)c1)C(=O)O]

As shown throughout the disclosure, the skilled artisan would appreciate that the compounds described therein can be in a stereoisomeric form. In embodiments, the compound of Formula (VIID-1) is represented by the stereoisomer of Formula (VIID-1):

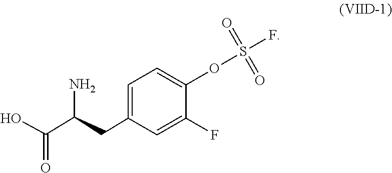 [SMILES: N[C@@H](Cc1ccc(OS(=O)(=O)F)c(F)c1)C(=O)O]

### RNA-Binding Proteins

Provided herein are RNA-binding protein comprises an unnatural amino acid, wherein the unnatural amino acid comprises a side chain of Formula (II):

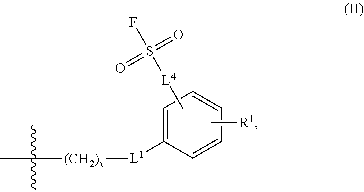

wherein L⁴ is a bond or —O—; and R¹, L¹, and x are as defined herein. In embodiments, L⁴ is a bond. In embodiments, L⁴ is —O—. In embodiments, R¹ is an electron-donating group or an electron-withdrawing group. In embodiments, when L⁴ is a bond then R¹ is an electron-donating group. In embodiments, when L⁴ is —O— then R¹ is an electron-withdrawing group. In embodiments -L⁴S(═O)₂F is para, meta, or ortho to the carbon atom linked to L¹. In embodiments L⁴S(═O)₂F is para to the carbon atom linked to L¹. In embodiments L⁴S(═O)₂F is meta to the carbon atom linked to L¹. In embodiments -L⁴S(═O)₂F is ortho to the carbon atom linked to L. In embodiments, R¹ is ortho, meta, or para to -L⁴S(═O)₂F. In embodiments, R′ is ortho to -L⁴S(═O)₂F. In embodiments, R¹ is meta to -L⁴S(═O)₂F. In embodiments, R¹ is para to -L⁴S(═O)₂F. In embodiments, the RNA-binding protein is a CRISPR protein or an RNA chaperone.

Provided herein are RNA-binding protein comprises an unnatural amino acid, wherein the unnatural amino acid comprises a side chain of Formula (IIA):

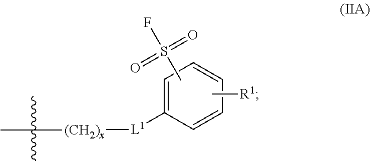

wherein R¹, L¹, and x are as defined herein. In embodiments, the RNA-binding protein is a CRISPR protein or an RNA chaperone. In embodiments, R¹ is an electron-donating group.

Provided herein are RNA-binding protein comprises an unnatural amino acid, wherein the unnatural amino acid comprises a side chain of Formula (IIB):

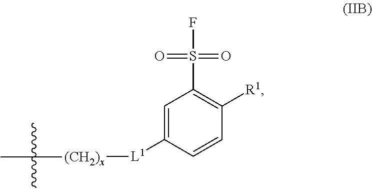

wherein R¹, L¹, and x are as defined herein. In embodiments, the RNA-binding protein is a CRISPR protein or an RNA chaperone. In embodiments, R¹ is an electron-donating group.

Provided herein are RNA-binding protein comprises an unnatural amino acid, wherein the unnatural amino acid comprises a side chain of Formula (IIC):

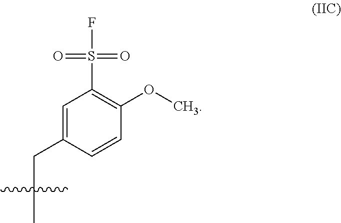 [SMILES: *Cc1ccc(OC)c(S(=O)(=O)F)c1]

Provided herein are RNA-binding protein comprises an unnatural amino acid, wherein the unnatural amino acid comprises a side chain of Formula ID):

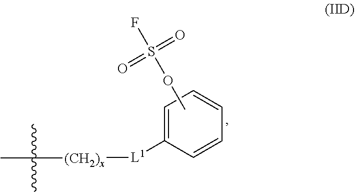

wherein L¹ and x are as defined herein. In embodiments —OS(═O)₂F is para, meta, or ortho to the carbon atom linked to L. In embodiments —OS(═O)₂F is para to the carbon atom linked to L¹. In embodiments —OS(═O)₂F is meta to the carbon atom linked to L¹. In embodiments —OS(═O)₂F is ortho to the carbon atom linked to L. In embodiments, the RNA-binding protein is a CRISPR protein or an RNA chaperone.

Provided herein are RNA-binding protein comprises an unnatural amino acid, wherein the unnatural amino acid comprises a side chain of Formula (IIE):

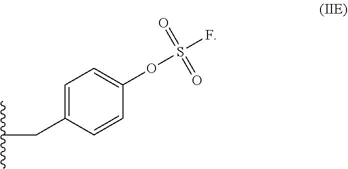 [SMILES: *Cc1ccc(OS(=O)(=O)F)cc1]

In embodiments, the RNA-binding protein is a CRISPR protein or an RNA chaperone.

In embodiments, the RNA-binding protein comprises any unnatural amino acid described herein. In embodiments, the RNA-binding protein comprises the unnatural amino acid of Formula (I). In embodiments, the RNA-binding protein comprises the unnatural amino acid of Formula (IA). In embodiments, the RNA-binding protein comprises the unnatural amino acid of Formula (IB). In embodiments, the RNA-binding protein comprises the unnatural amino acid of Formula (IC). In embodiments, the RNA-binding protein comprises the unnatural amino acid of Formula (ID). In embodiments, the RNA-binding protein comprises the unnatural amino acid of Formula (IE). In embodiments, the RNA-binding protein comprises the unnatural amino acid of Formula (IV). In embodiments, the RNA-binding protein comprises the unnatural amino acid of Formula (IVA). In embodiments, the RNA-binding protein comprises the unnatural amino acid of Formula (IVB). In embodiments, the RNA-binding protein comprises the unnatural amino acid of Formula (VII). In embodiments, the RNA-binding protein comprises the unnatural amino acid of Formula (VIIA). In embodiments, the RNA-binding protein comprises the unnatural amino acid of Formula (VIIB). In embodiments, the RNA-binding protein comprises the unnatural amino acid of Formula (VIIC). In embodiments, the RNA-binding protein comprises the unnatural amino acid of Formula (VIID). In embodiments, the RNA-binding protein comprises the unnatural amino acid of Formula (IVB). In embodiments, the RNA-binding protein comprises an unnatural amino acid, wherein the unnatural amino acid comprises a side chain as described herein. In embodiments, the RNA-binding protein comprises an unnatural amino acid, wherein the unnatural amino acid comprises a side chain of Formula (II). In embodiments, the RNA-binding protein comprises an unnatural amino acid, wherein the unnatural amino acid comprises a side chain of Formula (V). In embodiments, the RNA-binding protein comprises an unnatural amino acid, wherein the unnatural amino acid comprises a side chain of Formula (IE-A). In embodiments, the RNA-binding protein comprises an unnatural amino acid, wherein the unnatural amino acid comprises a side chain of Formula (VA). In embodiments, the RNA-binding protein comprises an unnatural amino acid, wherein the unnatural amino acid comprises a side chain of Formula (VIIIC). In embodiments, the RNA-binding protein comprises an unnatural amino acid, wherein the unnatural amino acid comprises a side chain of Formula (VB).

In embodiments, the RNA-binding protein is a CRISPR protein. In embodiments, the CRISPR protein is dCas3, dCas4, dCs5, dCas8, dCas9, dCas10, dCas12, or dCas13. In embodiments, the CRISPR protein is dCas3, dCas4, dCas5, dCas8a, dCas8b, dCas8c, dCas9, dCs10d, dCas12a, dCas12b, dCas12c, dCas12d, dCas12e, dCas12f, dCas12g, dCas12h, dCas12i, dCas12k, dCas13a, dCas13b, dCas13c, dCas13d, ddCpf1, dLbCpf1, dFnCpf1, dCas-phi, dCsn2, or dCse2. In embodiments, the CRISPR protein is dCas8a, dCas8b, dCas8c, dCas9, dCs10d, dCas12a, dCas12b, dCas12c, dCas12d, dCas12e, dCas12f, dCas12g, dCas12h, dCas12i, dCas12k, dCas13a, dCas13b, dCas13c, or dCas13d. In embodiments, the CRISPR protein is dCas9. In embodiments, the CRISPR protein is dCas13. In embodiments, the CRISPR protein is dCas13c. In embodiments, the CRISPR protein is dCas12. In embodiments, the CRISPR protein is a nuclease-deficient Cas9 variant. In embodiments, the CRISPR protein is a nuclease-deficient Class II CRISPR endonuclease. In embodiments, the CRISPR protein is dCas3. In embodiments, the CRISPR protein is dCas4. In embodiments, the CRISPR protein is dCas8a. In embodiments, the CRISPR protein is dCas8b. In embodiments, the CRISPR protein is dCas5. In embodiments, the CRISPR protein is dCas10d. In embodiments, the CRISPR protein is dCsn2. In embodiments, the CRISPR protein is dCse1. In embodiments, the CRISPR protein is dCse2. In embodiments, the CRISPR protein is dCas12b. In embodiments, the CRISPR protein is dCas12c. In embodiments, the CRISPR protein is dCas12d. In embodiments, the CRISPR protein is dCas12e. In embodiments, the CRISPR protein is dCas12f. In embodiments, the CRISPR protein is dCas12g. In embodiments, the CRISPR protein is dCas12h. In embodiments, the CRISPR protein is dCas12i. In embodiments, the CRISPR protein is dCas12k. In embodiments, the CRISPR protein is ddCpf1. In embodiments, the CRISPR protein is dLbCpf1. In embodiments, R² is dFnCpf1. In embodiments, the CRISPR protein is dCas-phi. In embodiments, the CRISPR protein comprises the unnatural amino acid sidechain at a position corresponding to position 128, 133, 380, 1053, 1058 (with reference to the amino acid sequence of catalytically inactive Cas13b from Prevotella sp. P5-125, e.g., any one of SEQ ID NOS:2-4)). In embodiments, the CRISPR protein comprises the unnatural amino acid sidechain at a position corresponding to position 128 (with reference to the amino acid sequence of any one of SEQ ID NOS:2-4). In embodiments, the CRISPR protein comprises the unnatural amino acid sidechain at a position corresponding to position 133 (with reference to the amino acid sequence of any one of SEQ ID NOS:2-4). In embodiments, the CRISPR protein comprises the unnatural amino acid sidechain at a position corresponding to position 380 (with reference to the amino acid sequence of any one of SEQ ID NOS:2-4). In embodiments, the CRISPR protein comprises the unnatural amino acid sidechain at a position corresponding to position 1053 (with reference to the amino acid sequence of any one of SEQ ID NOS:2-4). In embodiments, the CRISPR protein comprises the unnatural amino acid sidechain at a position corresponding to position 1058 (with reference to the amino acid sequence of any one of SEQ ID NOS:2-4).

In embodiments, the CRISPR protein is a catalytically inactive Cas13b (dCas13b). In embodiments, the CRISPR protein is dCas13b from Prevotella sp. P5-125 (dPsCas13b), from Bergeyella zoohelcum, or from Prevotella buccae. In embodiments, the catalytically inactive Cas13b protein comprises the unnatural amino acid sidechain at a position corresponding to position 133 or 380. In embodiments, the catalytically inactive Cas13b protein comprises the unnatural amino acid sidechain at a position corresponding to position 133. In embodiments, the catalytically inactive Cas13b protein comprises the unnatural amino acid sidechain at a position corresponding to position 380. In embodiments, the catalytically inactive Cas13b protein comprises the unnatural amino acid sidechain at a position corresponding to position 128, 133, 380, 1053, 1058, or two or more thereof. In embodiments, the catalytically inactive Cas13b protein comprises the unnatural amino acid sidechain at a position corresponding to position 116, 121, 128, 133, 156, 161, 380, 393, 402, 459, 1053, 1058, 1068, 1072, 1177, 1182, or two or more thereof.

In embodiments, the CRISPR protein is dCas13b from Prevotella sp. P5-125 (dPsCas13b). In embodiments, the catalytically inactive Cas13b protein from Prevotella sp. P5-125 comprises the unnatural amino acid sidechain at a position corresponding to position R128, H133, R380, R1053, H1058, or two or more thereof. In embodiments, the catalytically inactive Cas13b protein from Prevotella sp. P5-125 comprises the unnatural amino acid sidechain at a position corresponding to position H133 or R380. In embodiments, the catalytically inactive Cas13b protein from Prevotella sp. P5-125 comprises the unnatural amino acid sidechain at a position corresponding to position 133 or 380. In embodiments, the catalytically inactive Cas13b protein from Prevotella sp. P5-125 comprises the unnatural amino acid sidechain at a position corresponding to position 133. In embodiments, the catalytically inactive Cas13b protein from Prevotella sp. P5-125 comprises the unnatural amino acid sidechain at a position corresponding to position 380. In embodiments, the catalytically inactive Cas13b protein from Prevotella sp. P5-125 comprises the unnatural amino acid sidechain at a position corresponding to position R128. In embodiments, the catalytically inactive Cas13b protein from Prevotella sp. P5-125 comprises the unnatural amino acid sidechain at a position corresponding to position H133. In embodiments, the catalytically inactive Cas13b protein from Prevotella sp. P5-125 comprises the unnatural amino acid sidechain at a position corresponding to position R380. In embodiments, the catalytically inactive Cas13b protein from Prevotella sp. P5-125 comprises the unnatural amino acid sidechain at a position corresponding to position R1053. In embodiments, the catalytically inactive Cas13b protein from Prevotella sp. P5-125 comprises the unnatural amino acid sidechain at a position corresponding to position H1058.

The amino acid sequence for the catalytically active Cas13b protein from Prevotella sp. P5-125 is SEQ ID NO:45. The catalytically active Cas13b protein from Prevotella sp. P5-125 is a catalytically inactive Cas13b protein from Prevotella sp. P5-125 when H133 is mutated to Ala (SEQ ID NO:46), when H1058 is mutated to Ala (SEQ ID NO:47), or when H133 and H1058 are mutated to Ala (SEQ ID NO:48).

In embodiments, the CRISPR protein is dCas13b from Bergeyella zoohelcum. In embodiments, the catalytically inactive Cas13b protein from Bergeyella zoohelcum comprises the unnatural amino acid sidechain at a position corresponding to position R116, H121, R459, R1177, H1182, or two or more thereof. In embodiments, the catalytically inactive Cas13b protein from Bergeyella zoohelcum comprises the unnatural amino acid sidechain at a position corresponding to position R116. In embodiments, the catalytically inactive Cas13b protein from Bergeyella zoohelcum comprises the unnatural amino acid sidechain at a position corresponding to position H121. In embodiments, the catalytically inactive Cas13b protein from Bergeyella zoohelcum comprises the unnatural amino acid sidechain at a position corresponding to position R459. In embodiments, the catalytically inactive Cas13b protein from Bergeyella zoohelcum comprises the unnatural amino acid sidechain at a position corresponding to position R1177. In embodiments, the catalytically inactive Cas13b protein from Bergeyella zoohelcum comprises the unnatural amino acid sidechain at a position corresponding to position H1182.

In embodiments, the CRISPR protein is dCas13b from Prevotella buccae. In aspects, the catalytically inactive Cas13b protein from Prevotella buccae comprises the unnatural amino acid sidechain at a position corresponding to position R156, H161, K393, R402, R1068, H1073, or two or more thereof. In embodiments, the catalytically inactive Cas13b protein from Prevotella buccae comprises the unnatural amino acid sidechain at a position corresponding to position R156. In embodiments, the catalytically inactive Cas13b protein from Prevotella buccae comprises the unnatural amino acid sidechain at a position corresponding to position H161. In embodiments, the catalytically inactive Cas13b protein from Prevotella buccae comprises the unnatural amino acid sidechain at a position corresponding to position K393. In embodiments, the catalytically inactive Cas13b protein from Prevotella buccae comprises the unnatural amino acid sidechain at a position corresponding to position R402. In embodiments, the catalytically inactive Cas13b protein from Prevotella buccae comprises the unnatural amino acid sidechain at a position corresponding to position R1068. In embodiments, the catalytically inactive Cas13b protein from Prevotella buccae comprises the unnatural amino acid sidechain at a position corresponding to position H1073.

In embodiments, the CRISPR protein is a catalytically inactive Cas13a protein (dCas13a). In embodiments, the CRISPR protein is a catalytically inactive Cas13a protein from Leptotrichia buccalis or Leptotrichia wadei. In embodiments, the catalytically inactive Cas13a protein comprises the unnatural amino acid sidechain at a position corresponding to position 47, 472, 473, 474, 475, 477, 479, 522, 524, 586, 590, 653, 659, 808, 810, 853, 855, 902, 904, 1046, 1051, 1053, 1133, 1135, or two or more thereof.

In embodiments, the CRISPR protein is a catalytically inactive Cas13a protein from Leptotrichia buccalis. In embodiments, the catalytically inactive Cas13a protein from Leptotrichia buccalis comprises the unnatural amino acid sidechain at a position corresponding to position K47, R472, H473, H477, S522, D590, Q659, V810, K855, Q904, R1046, H1053, R1135, or two or more thereof.

In embodiments, the CRISPR protein is a catalytically inactive Cas13a protein is from Leptotrichia wadei. In embodiments, the catalytically inactive Cas13a protein from Leptotrichia wadei comprises the unnatural amino acid sidechain at a position corresponding to position K47, R474, H475, H479, S524, D586, Q653, V808, K853, Q902, R1046, H1051, R1133, or two or more thereof.

In embodiments, the CRISPR protein is a catalytically inactive Cas13d (dCas13d). In embodiments, the CRISPR protein is a catalytically inactive Cas13d protein from Eubacterium siraeum. In embodiments, the catalytically inactive Cas13d protein comprises the unnatural amino acid sidechain at a position corresponding to position 84, 86, 386, 405, 524, 641, 679, 680, or two or more thereof. In embodiments, the catalytically inactive Cas13d protein from Eubacterium siraeum comprises the unnatural amino acid sidechain at a position corresponding to position R84, N86, R386, N405, T524, N641, R679, Y680, or two or more thereof.

In embodiments, the CRISPR protein is a catalytically inactive Cas12a (dCas12a). In embodiments, the CRISPR protein is a catalytically inactive Cas12a protein from Acidaminococcus sp. BV3L6, Lachnospiraceae bacterium ND2006, or Francisella novicida U112. In embodiments, the catalytically inactive Cas12a protein comprises the unnatural amino acid sidechain at a position corresponding to position 833, 908, 917, 926, 993, 1006, 1139, 1149, 1181, 1218, 1226, 1255, 1263, 1226, 1235, or two or more thereof

In embodiments, the CRISPR protein is a catalytically inactive Cas12a protein from Acidaminococcus sp. BV3L6. In embodiments, the catalytically inactive Cas12a protein is from Acidaminococcus sp. BV3L6 comprises the unnatural amino acid sidechain at a position corresponding to position D908, E993, D1263, R1226, D1235, or two or more thereof

In embodiments, the CRISPR protein is a catalytically inactive Cas12a protein from Lachnospiraceae bacterium ND2006. In embodiments, the catalytically inactive Cas12a protein from Lachnospiraceae bacterium ND2006 comprises the unnatural amino acid sidechain at a position corresponding to position D833, E926, D1181, R1139, D1149, or two or more thereof

In embodiments, the CRISPR protein is a catalytically inactive Cas12a protein from Francisella novicida U112. In embodiments, the catalytically inactive Cas12a protein from Francisella novicida U112 comprises the unnatural amino acid sidechain at a position corresponding to position D917, E1006, D1255, R1218, D1226, or two or more thereof

In embodiments, the CRISPR protein is a catalytically inactive Cas9 protein (dCas9). In embodiments, the CRISPR protein is a catalytically inactive Cas9 protein from Streptococcus pyogenes, Staphylococcus aureus, or Actinomyces naeslundii. In embodiments, the CRISPR protein is a catalytically inactive Cas9 protein from Streptococcus pyogenes, Staphylococcus aureus, or Actinomyces naeslundii. In embodiments, the catalytically inactive Cas9 protein comprises the unnatural amino acid sidechain at a position corresponding to position 10, 17, 477, 505, 556, 557, 580, 581, 582, 606, 701, 704, 736, 739, 762, 983, 986, 840, 863, 839, or two or more thereof.

In embodiments, the CRISPR protein is a catalytically inactive Cas9 protein from Streptococcus pyogenes. In embodiments, the catalytically inactive Cas9 protein from Streptococcus pyogenes comprises the unnatural amino acid sidechain at a position corresponding to position D10, E762, H983, D986, H840, N863, D839, or two or more thereof.

In embodiments, the CRISPR protein is a catalytically inactive Cas9 protein from Staphylococcus aureus. In embodiments, the catalytically inactive Cas9 protein from Staphylococcus aureus comprises the unnatural amino acid sidechain at a position corresponding to position D10, E477, H701, D704, H557, N580, D556, or two or more thereof.

In embodiments, the CRISPR protein is a catalytically inactive Cas9 protein from Actinomyces naeslundii. In embodiments, the catalytically inactive Cas9 protein from Actinomyces naeslundii comprises the unnatural amino acid sidechain at a position corresponding to position D17, E505, H736, D739, H582, N606, D581, or two or more thereof.

In embodiments, the RNA-binding protein is an RNA chaperone. In embodiments, the RNA chaperone is a Hfq protein. In embodiments, the Hfq protein comprises the unnatural amino acid sidechain at a position corresponding to position 25, position 30, or position 49. In embodiments, the Hfq protein comprises the unnatural amino acid sidechain at a position corresponding to position 25. In embodiments, the Hfq protein comprises the unnatural amino acid sidechain at a position corresponding to position 30. In embodiments, the Hfq protein comprises the unnatural amino acid sidechain at a position corresponding to position 49.

### Proteins

Provided herein are proteins comprising an unnatural amino acid, wherein the unnatural amino acid comprises a side chain of Formula (V):

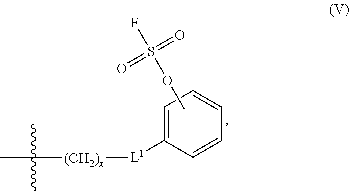

wherein —OS(═O)₂F is meta or ortho to the carbon atom linked to L¹; x is an integer from 1 to 8; and L¹ is a bond, substituted or unsubstituted alkylene, or substituted or unsubstituted heteroalkylene. In embodiments, —OS(═O)₂F is ortho to the carbon atom linked to L¹. In embodiments, —OS(═O)₂F is meta to the carbon atom linked to L¹. In embodiments, the protein is an antibody, an antibody variant, or a receptor protein.

Provided herein are proteins comprising an unnatural amino acid, wherein the unnatural amino acid comprises a side chain of Formula (VA):

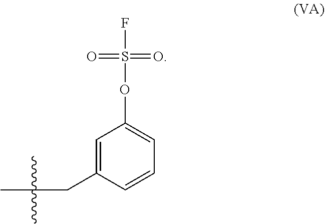 [SMILES: *Cc1cccc(OS(=O)(=O)F)c1]

In embodiments, the protein is an antibody, an antibody variant, or a receptor protein.

Provided herein are proteins comprising an unnatural amino acid, wherein the unnatural amino acid comprises a side chain of Formula (VB):

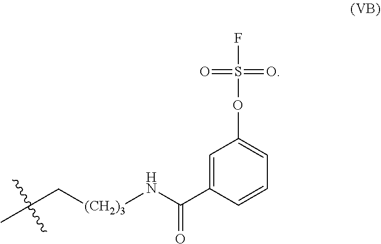

In embodiments, the protein is an antibody, an antibody variant, or a receptor protein.

Provided herein are proteins comprising unnatural amino acids, wherein the unnatural amino acid side chain is represented by the structure of Formula (VIII):

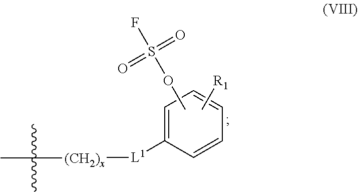

wherein R¹, L¹, and x are as defined herein. In embodiments, R¹ is an electron-withdrawing group. In embodiments, —OS(═O)₂F is ortho, meta, or para to the carbon atom linked to L¹. In embodiments, —OS(═O)₂F is ortho to the carbon atom linked to L¹. In embodiments, —OS(═O)₂F is meta to the carbon atom linked to L¹. In embodiments, —OS(═O)₂F is para to the carbon atom linked to L¹. In embodiments, R¹ is ortho, meta, or para to —OS(═O)₂F. In embodiments, R¹ is ortho to —OS(═O)₂F. In embodiments, R¹ is meta to —OS(═O)₂F. In embodiments, R¹ is para to —OS(═O)₂F. In embodiments, the protein is an antibody, an antibody variant, or a receptor protein.

In embodiments, the unnatural amino acid side chain is represented by the structure of Formula (VIIIA):

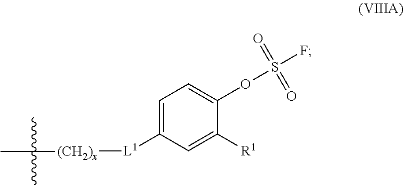

wherein R¹, L¹, and x are as defined herein. In embodiments, the protein is an antibody, an antibody variant, or a receptor protein. In embodiments, R¹ is an electron-withdrawing group.

In embodiments, the unnatural amino acid side chain is represented by the structure of Formula (VIIIB):

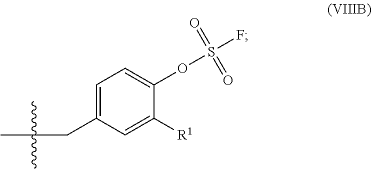

wherein R¹ is as defined herein. In embodiments, R¹ is an electron-withdrawing group.

In embodiments, the unnatural amino acid is FFY and the unnatural amino acid side chain is represented by the structure of Formula (VIIIC):

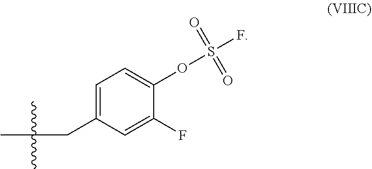 [SMILES: *Cc1ccc(OS(=O)(=O)F)c(F)c1]

In embodiments of the compounds described herein, the protein is an antibody, an antibody variant, or a receptor protein. In embodiments, the protein is an antibody. In embodiments, the protein is an antibody variant. In embodiments, the protein is a receptor protein. In embodiments, the antibody variant is a variant as defined herein. In embodiments, the antibody variant is a single-chain variable fragment, a single-domain antibody, an affibody, or an antigen-binding fragment. In embodiments, the antibody variant is a single-chain variable fragment. In embodiments, the antibody variant is a single-domain antibody. In embodiments, the antibody variant is an affibody. In embodiments, the antibody variant is or an antigen-binding fragment. In embodiments, the receptor protein is any receptor protein described herein.

In embodiments of the compounds described herein, the protein is a receptor protein. In embodiments, the receptor protein is a programmed death-ligand 1 (PD-L1) receptor, a programmed cell death protein 1 (PD-1) receptor, a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid S1P receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, or a combination of two or more thereof.

In embodiments of the compounds described herein, the protein is a receptor protein. In embodiments, the receptor protein is a programmed death-ligand 1 (PD-L1) receptor, a programmed cell death protein 1 (PD-1) receptor, a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SiP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, or a combination of two or more thereof. In embodiments, the receptor protein is an integrin. In embodiments, the receptor protein is a somatostain receptor. In embodiments, the receptor protein is a gonadotropin-releasing hormone receptor. In embodiments, the receptor protein is a bombesin receptor. In embodiments, the receptor protein is a vasoactive intestinal peptide receptor. In embodiments, the receptor protein is a neurotensin receptor. In embodiments, the receptor protein is a cholecystokinin 2 receptor. In embodiments, the receptor protein is a melanocortin receptor. In embodiments, the receptor protein is a ghrelin receptor.

In embodiments, the receptor protein is a PD-L1 receptor or a PD-1 receptor. In embodiments, the receptor protein is a PD-L1 receptor. In embodiments, the receptor protein is a PD-1 receptor.

In embodiments, the receptor protein is a receptor expressed on a cancer cell. In embodiments, the receptor protein is a receptor overexpressed on a cancer cell relative to a control.

In embodiments, the receptor protein is a G protein-coupled receptor. In embodiments, the receptor protein is a receptor tyrosine kinase. In embodiments, the receptor protein is a an ErbB receptor. In embodiments, the receptor protein is an epidermal growth factor receptor (EGFR). In embodiments, the receptor protein is epidermal growth factor receptor 1 (HER1). In embodiments, the receptor protein is epidermal growth factor receptor 2 (HER2). In embodiments, the receptor protein is epidermal growth factor receptor 3 (HER3). In embodiments, the receptor protein is epidermal growth factor receptor 4 (HER4).

### Conjugates

Provided herein are RNA-binding protein/RNA conjugates of Formula (III):

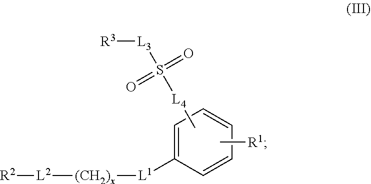

where R² is a RNA-binding protein, R³ is RNA, L⁴ is a bond or —O—; and R′, L¹, L², L³, and x are as defined herein. In embodiments, L⁴ is a bond. In embodiments, L⁴ is —O—. In embodiments, R¹ is an electron-donating group or an electron-withdrawing group. In embodiments, when L⁴ is a bond then R¹ is an electron-donating group. In embodiments, when L⁴ is —O— then R¹ is an electron-withdrawing group. In embodiments, the RNA-binding protein is a CRISPR protein or an RNA chaperone.

Provided herein are RNA-binding protein/RNA conjugates of Formula (IIIA):

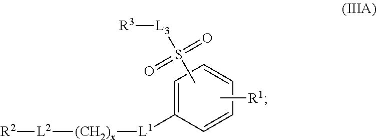

where R² is a RNA-binding protein, R³ is RNA, and R¹, L¹, L², L³, and x are as defined herein. In embodiments, the RNA-binding protein is a CRISPR protein or an RNA chaperone. In embodiments, R¹ is an electron-donating group.

Provided herein are RNA-binding protein/RNA conjugates of Formula (IIIB):

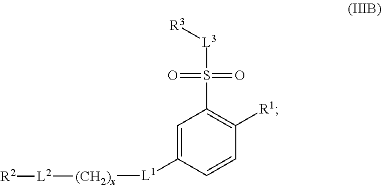

where R² is a RNA-binding protein, R³ is RNA, and R¹, L¹, L², L³, and x are as defined herein. In embodiments, the RNA-binding protein is a CRISPR protein or an RNA chaperone. In embodiments, R¹ is an electron-donating group.

Provided herein are RNA-binding protein/RNA conjugates of Formula (IIIC):

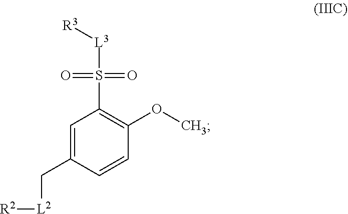

Where R² is a RNA-binding protein, R³ is RNA, and L², and L³ are as defined herein. In embodiments, the RNA-binding protein is a CRISPR protein or an RNA chaperone.

Provided herein are RNA-binding protein/RNA conjugates of Formula (IIID):

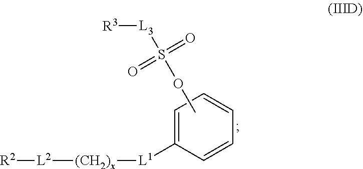

where R² is a RNA-binding protein, R³ is RNA, and L², L³, and x are as defined herein. In embodiments, the RNA-binding protein is a CRISPR protein or an RNA chaperone.

Provided herein are RNA-binding protein/RNA conjugates of Formula (IIIE):

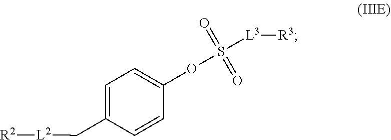

where R² is a RNA-binding protein, R³ is RNA, and L² and L³ are as defined herein. In embodiments, the RNA-binding protein is a CRISPR protein or an RNA chaperone.

In embodiments of the compounds described herein, R² is a RNA-binding protein. In embodiments of the compounds described herein, R² is a CRISPR protein or an RNA chaperone.

In embodiments, R² is an RNA chaperone. In embodiments, the RNA chaperone is Hfq protein. In embodiments, the Hfq protein comprises the unnatural amino acid sidechain at a position corresponding to position 25, position 30, or position 49. In embodiments, the Hfq protein comprises the unnatural amino acid sidechain at a position corresponding to position 25. In embodiments, the Hfq protein comprises the unnatural amino acid sidechain at a position corresponding to position 30. In embodiments, the Hfq protein comprises the unnatural amino acid sidechain at a position corresponding to position 49. In embodiments, L² is bonded to the RNA chaperone.

In embodiments, R² is a CRISPR protein. In embodiments, R² is dCas. In embodiments, R² is dCas3, dCas4, dCs5, dCas8, dCas9, dCas10, dCas12, or dCas13. In embodiments, R² is dCas3, dCas4, dCas5, dCas8a, dCas8b, dCas8c, dCas9, dCs10d, dCas12a, dCas12b, dCas12c, dCas12d, dCas12e, dCas12f, dCas12g, dCas12h, dCas12i, dCas12k, dCas13a, dCas13b, dCas13c, dCas13d, ddCpf1, dLbCpf1, dFnCpf1, dCas-phi, dCsn2, or dCse2. In embodiments, R² is dCas8a, dCas8b, dCas8c, dCas9, dCs10d, dCas12a, dCas12b, dCas12c, dCas12d, dCas12e, dCas12f, dCas12g, dCas12h, dCas12i, dCas12k, dCas13a, dCas13b, dCas13c, or dCas13d. In embodiments, R² is dCas9. In embodiments, R² is dCas13. In embodiments, R² is dCas13c. In embodiments, R² is dCas12. In embodiments, R² is a nuclease-deficient Cas9 variant. In embodiments, R² is a nuclease-deficient Class II CRISPR endonuclease. In embodiments, R² is dCas3. In embodiments, R² is dCas4. In embodiments, R² is dCas8a. In embodiments, R² is dCas8b. In embodiments, R² is dCas5. In embodiments, R² is dCas10d. In embodiments, R² is dCsn2. In embodiments, R² is dCse1. In embodiments, R² is dCse2. In embodiments, R² is dCas12b. In embodiments, R² is dCas12c. In embodiments, R² is dCas12d. In embodiments, R² is dCas12e. In embodiments, R² is dCas12f. In embodiments, R² is dCas12g. In embodiments, R² is dCas12h. In embodiments, R² is dCas12i. In embodiments, R² is dCas12k. In embodiments, R² is ddCpf1. In embodiments, R² is dLbCpf1. In embodiments, R² is dFnCpf1. In embodiments, R² is dCas-phi. In embodiments, L² is bonded to the CRISPR protein. In embodiments, R² comprises the unnatural amino acid sidechain at a position corresponding to position 128, 133, 380, 1053, 1058 (with reference to the amino acid sequence of any one of SEQ ID NOS:46-48). In embodiments, R² comprises the unnatural amino acid sidechain at a position corresponding to position 128 (with reference to the amino acid sequence of any one of SEQ ID NOS:46-48). In embodiments, R² comprises the unnatural amino acid sidechain at a position corresponding to position 133 (with reference to the amino acid sequence of any one of SEQ ID NOS:46-48). In embodiments, R² comprises the unnatural amino acid sidechain at a position corresponding to position 380 (with reference to the amino acid sequence of any one of SEQ ID NOS:46-48). In embodiments, R² comprises the unnatural amino acid sidechain at a position corresponding to position 1053 (with reference to the amino acid sequence of any one of SEQ ID NOS:46-48). In embodiments, R² comprises the unnatural amino acid sidechain at a position corresponding to position 1058 (with reference to the amino acid sequence of any one of SEQ ID NOS:46-48).

In embodiments, R² is a catalytically inactive Cas13a protein (dCas13a). In embodiments, the CRISPR protein is catalytically inactive Cas13a protein from Leptotrichia buccalis or Leptotrichia wadei. In embodiments, R² is catalytically inactive Cas13a protein from Leptotrichia buccalis or Leptotrichia wadei. In embodiments, the catalytically inactive Cas13a protein comprises the unnatural amino acid sidechain at a position corresponding to position 47, 472, 473, 474, 475, 477, 479, 522, 524, 586, 590, 653, 659, 808, 810, 853, 855, 902, 904, 1046, 1051, 1053, 1133, 1135, or two or more thereof. In embodiments, R² is a catalytically inactive Cas13a protein from Leptotrichia buccalis. In embodiments, the catalytically inactive Cas13a protein from Leptotrichia buccalis comprises the unnatural amino acid sidechain at a position corresponding to position K47, R472, H473, H477, 5522, D590, Q659, V810, K855, Q904, R1046, H1053, R1135, or two or more thereof. In embodiments, R² is a catalytically inactive Cas13a protein is from Leptotrichia wadei. In embodiments, the catalytically inactive Cas13a protein from Leptotrichia wadei comprises the unnatural amino acid sidechain at a position corresponding to position K47, R474, H475, H479, 5524, D586, Q653, V808, K853, Q902, R1046, H1051, R1133, or two or more thereof.

In embodiments, R² is a catalytically inactive Cas13b (dCas13b). In embodiments, R² is dCas13b from Prevotella sp. P5-125 (dPsCas13b), from Bergeyella zoohelcum, or from Prevotella buccae. In embodiments, the catalytically inactive Cas13b protein comprises the unnatural amino acid sidechain at a position corresponding to position 133 or 380. In embodiments, the catalytically inactive Cas13b protein comprises the unnatural amino acid sidechain at a position corresponding to position 133. In embodiments, the catalytically inactive Cas13b protein comprises the unnatural amino acid sidechain at a position corresponding to position 380. In embodiments, the catalytically inactive Cas13b protein comprises the unnatural amino acid sidechain at a position corresponding to position 128, 133, 380, 1053, 1058, or two or more thereof. In embodiments, the catalytically inactive Cas13b protein comprises the unnatural amino acid sidechain at a position corresponding to position 116, 121, 128, 133, 156, 161, 380, 393, 402, 459, 1053, 1058, 1068, 1072, 1177, 1182, or two or more thereof.

In embodiments, R² is dCas13b from Prevotella sp. P5-125 (dPsCas13b). In embodiments, the catalytically inactive Cas13b protein from Prevotella sp. P5-125 comprises the unnatural amino acid sidechain at a position corresponding to position R128, H133, R380, R1053, H1058, or two or more thereof. In embodiments, the catalytically inactive Cas13b protein from Prevotella sp. P5-125 comprises the unnatural amino acid sidechain at a position corresponding to position H133 or R380. In embodiments, the catalytically inactive Cas13b protein from Prevotella sp. P5-125 comprises the unnatural amino acid sidechain at a position corresponding to position 133 or 380. In embodiments, the catalytically inactive Cas13b protein from Prevotella sp. P5-125 comprises the unnatural amino acid sidechain at a position corresponding to position 133. In embodiments, the catalytically inactive Cas13b protein from Prevotella sp. P5-125 comprises the unnatural amino acid sidechain at a position corresponding to position 380. In embodiments, the catalytically inactive Cas13b protein from Prevotella sp. P5-125 comprises the unnatural amino acid sidechain at a position corresponding to position R128. In embodiments, the catalytically inactive Cas13b protein from Prevotella sp. P5-125 comprises the unnatural amino acid sidechain at a position corresponding to position H133. In embodiments, the catalytically inactive Cas13b protein from Prevotella sp. P5-125 comprises the unnatural amino acid sidechain at a position corresponding to position R380. In embodiments, the catalytically inactive Cas13b protein from Prevotella sp. P5-125 comprises the unnatural amino acid sidechain at a position corresponding to position R1053. In embodiments, the catalytically inactive Cas13b protein from Prevotella sp. P5-125 comprises the unnatural amino acid sidechain at a position corresponding to position H1058.

In embodiments, R² is dCas13b from Bergeyella zoohelcum. In embodiments, the catalytically inactive Cas13b protein from Bergeyella zoohelcum comprises the unnatural amino acid sidechain at a position corresponding to position R116, H121, R459, R1177, H1182, or two or more thereof. In embodiments, the catalytically inactive Cas13b protein from Bergeyella zoohelcum comprises the unnatural amino acid sidechain at a position corresponding to position R116. In embodiments, the catalytically inactive Cas13b protein from Bergeyella zoohelcum comprises the unnatural amino acid sidechain at a position corresponding to position H121. In embodiments, the catalytically inactive Cas13b protein from Bergeyella zoohelcum comprises the unnatural amino acid sidechain at a position corresponding to position R459. In embodiments, the catalytically inactive Cas13b protein from Bergeyella zoohelcum comprises the unnatural amino acid sidechain at a position corresponding to position R1177. In embodiments, the catalytically inactive Cas13b protein from Bergeyella zoohelcum comprises the unnatural amino acid sidechain at a position corresponding to position H1182.

In embodiments, R² is dCas13b from Prevotella buccae. In aspects, the catalytically inactive Cas13b protein from Prevotella buccae comprises the unnatural amino acid sidechain at a position corresponding to position R156, H161, K393, R402, R1068, H1073, or two or more thereof. In embodiments, the catalytically inactive Cas13b protein from Prevotella buccae comprises the unnatural amino acid sidechain at a position corresponding to position R156. In embodiments, the catalytically inactive Cas13b protein from Prevotella buccae comprises the unnatural amino acid sidechain at a position corresponding to position H161. In embodiments, the catalytically inactive Cas13b protein from Prevotella buccae comprises the unnatural amino acid sidechain at a position corresponding to position K393. In embodiments, the catalytically inactive Cas13b protein from Prevotella buccae comprises the unnatural amino acid sidechain at a position corresponding to position R402. In embodiments, the catalytically inactive Cas13b protein from Prevotella buccae comprises the unnatural amino acid sidechain at a position corresponding to position R1068. In embodiments, the catalytically inactive Cas13b protein from Prevotella buccae comprises the unnatural amino acid sidechain at a position corresponding to position H1073.

In embodiments, R² is a catalytically inactive Cas13d (dCas13d). In embodiments, R² is a catalytically inactive Cas13d protein from Eubacterium siraeum. In embodiments, the catalytically inactive Cas13d protein comprises the unnatural amino acid sidechain at a position corresponding to position 84, 86, 386, 405, 524, 641, 679, 680, or two or more thereof. In embodiments, the catalytically inactive Cas13d protein from Eubacterium siraeum comprises the unnatural amino acid sidechain at a position corresponding to position R84, N86, R386, N405, T524, N641, R679, Y680, or two or more thereof.

In embodiments, R² is a catalytically inactive Cas12a (dCas12a). In embodiments, the CRISPR protein is a catalytically inactive Cas12a protein is from Acidaminococcus sp. BV3L6, Lachnospiraceae bacterium ND2006, or Francisella novicida U112. In embodiments, R² is a catalytically inactive Cas12a protein is from Acidaminococcus sp. BV3L6, Lachnospiraceae bacterium ND2006, or Francisella novicida U112. In embodiments, the catalytically inactive Cas12a protein comprises the unnatural amino acid sidechain at a position corresponding to position 833, 908, 917, 926, 993, 1006, 1139, 1149, 1181, 1218, 1226, 1255, 1263, 1226, 1235, or two or more thereof. In embodiments, R² is a catalytically inactive Cas12a protein is from Acidaminococcus sp. BV3L6. In embodiments, the catalytically inactive Cas12a protein from Acidaminococcus sp. BV3L6 comprises the unnatural amino acid sidechain at a position corresponding to position D908, E993, D1263, R1226, D1235, or two or more thereof. In embodiments, R² is a catalytically inactive Cas12a protein is from Lachnospiraceae bacterium ND2006. In embodiments, the catalytically inactive Cas12a protein from Lachnospiraceae bacterium ND2006 comprises the unnatural amino acid sidechain at a position corresponding to position D833, E926, D1181, R1139, D1149, or two or more thereof. In embodiments, R² is a catalytically inactive Cas12a protein is from Francisella novicida U112. In embodiments, the catalytically inactive Cas12a protein from Francisella novicida U112 comprises the unnatural amino acid sidechain at a position corresponding to position D917, E1006, D1255, R1218, D1226, or two or more thereof

In embodiments, R² is a catalytically inactive Cas9 protein. In embodiments, the CRISPR protein is a catalytically inactive Cas9 protein from Streptococcus pyogenes, Staphylococcus aureus, or Actinomyces naeslundii. In embodiments, R² is a catalytically inactive Cas9 protein from Streptococcus pyogenes, Staphylococcus aureus, or Actinomyces naeslundii. In embodiments, the catalytically inactive Cas9 protein comprises the unnatural amino acid sidechain at a position corresponding to position 10, 17, 477, 505, 556, 557, 580, 581, 582, 606, 701, 704, 736, 739, 762, 983, 986, 840, 863, 839, or two or more thereof. In embodiments, R² is a catalytically inactive Cas9 protein from Streptococcus pyogenes. In embodiments, the catalytically inactive Cas9 protein from Streptococcus pyogenes comprises the unnatural amino acid sidechain at a position corresponding to position D10, E762, H983, D986, H840, N863, D839, or two or more thereof. In embodiments, R² is a catalytically inactive Cas9 protein from Staphylococcus aureus. In embodiments, the catalytically inactive Cas9 protein from Staphylococcus aureus comprises the unnatural amino acid sidechain at a position corresponding to position D10, E477, H701, D704, H557, N580, D556, or two or more thereof. In embodiments, R² is a catalytically inactive Cas9 protein from Actinomyces naeslundii. In embodiments, the catalytically inactive Cas9 protein from Actinomyces naeslundii comprises the unnatural amino acid sidechain at a position corresponding to position D17, E505, H736, D739, H582, N606, D581, or two or more thereof.

In embodiments of the compounds described herein, R³ is RNA. In embodiments, R³ is mRNA. In embodiments, R³ is sRNA. In embodiments, R³ is shRNA. In embodiments, R³ is siRNA. In embodiments, R³ is miRNA. In embodiments, R³ is tRNA. In embodiments, R³ is rRNA. In embodiments, L³ is bonded to a 2′-hydroxyl group of a ribose group or an amine group of the base of a nucleotide in the RNA. In embodiments, L³ is bonded to a 2′-hydroxyl group of a ribose group of a nucleotide in the RNA. In embodiments, L³ is bonded to a 2′-hydroxyl group of an amine group of the base of a nucleotide in the RNA. In embodiments, L³ is bonded to a 2′-hydroxyl group of a ribose group or an amine group of an adenine in the RNA. In embodiments, L³ is bonded to a 2′-hydroxyl group of a ribose group of an adenine in the RNA. In embodiments, L³ is bonded to a 2′-hydroxyl group of an amine group of an adenine in the RNA. In embodiments, L³ is bonded to a 2′-hydroxyl group of a ribose group or an amine group of a uracil in the RNA. In embodiments, L³ is bonded to a 2′-hydroxyl group of a ribose group of a uracil in the RNA. In embodiments, L³ is bonded to a 2′-hydroxyl group of an amine group of a uracil in the RNA. In embodiments, L³ is bonded to a 2′-hydroxyl group of a ribose group or an amine group of a guanine in the RNA. In embodiments, L³ is bonded to a 2′-hydroxyl group of a ribose group of a guanine in the RNA. In embodiments, L³ is bonded to a 2′-hydroxyl group of an amine group of a guanine in the RNA. In embodiments, L³ is bonded to a 2′-hydroxyl group of a ribose group or an amine group of a cytosine in the RNA. In embodiments, L³ is bonded to a 2′-hydroxyl group of a ribose group of a cytosine in the RNA. In embodiments, L³ is bonded to a 2′-hydroxyl group of an amine group of a cytosine in the RNA. In embodiments, the 2′-hydroxyl group in the ribose or amine group of the base is a nucleophilic 2′-hydroxyl group. In embodiments, L³ is a bond.

Provided herein are biomolecule conjugates comprising a first biomolecule moiety linked to a second biomolecule moiety by a bioconjugate liker of Formula (VI):

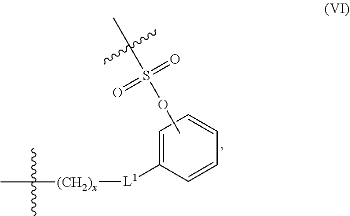

wherein —OS(═O)₂— is meta or ortho to the carbon atom linked to L¹, and L¹ and x are as defined herein. In embodiments, —OS(═O)₂— is meta to the carbon atom linked to L¹. In embodiments, —OS(═O)₂— is ortho to the carbon atom linked to L¹.

Provided herein are biomolecule conjugates of Formula (VIA):

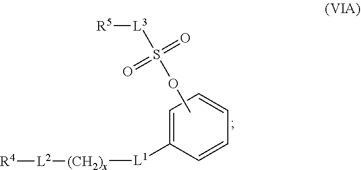

wherein —OS(═O)₂L³R⁵ is meta or ortho to the carbon atom linked to L¹; and R⁴ and R⁵ are each independently a peptidyl moiety, a carbohydrate moiety, or a nucleic acid moiety. In embodiments, R⁴ and R⁵ are each independently a peptidyl moiety. L¹, L², L³, and x have the same definition(s) as described herein. In embodiments, —OS(═O)₂L³R⁵ is meta to the carbon atom linked to L¹. In embodiments, —OS(═O)₂L³R⁵ is ortho to the carbon atom linked to L¹.

Provided herein are biomolecule conjugates of Formula (VIB):

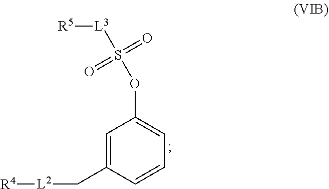

wherein R⁴ and R⁵ are each independently a peptidyl moiety, and L² and L³ have the same definition as described herein.

Provided herein are biomolecule conjugates of Formula (VIC):

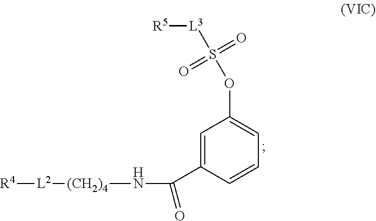

wherein R⁴ and R⁵ are each independently a peptidyl moiety, and L² and L³ have the same definitions as described herein.

Thus, the biomolecule of Formula (VIA) can be represented as follows when R⁵ is a peptidyl moiety comprising a histidine residue bonded to L³ when L³ is a bond:

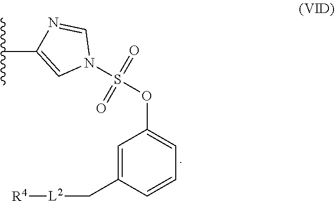

The biomolecule of Formula (VIA) can be represented as follows when R⁵ is a peptidyl moiety comprising a tyrosine residue bonded to L³ when L³ is a bond:

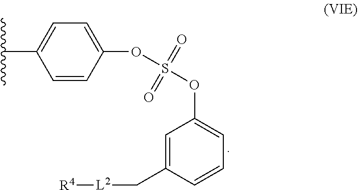

The biomolecule of Formula (VIA) can be represented as follows when R⁵ is a peptidyl moiety comprising a lysine residue bonded to L³ when L³ is a bond:

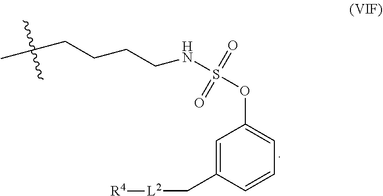

In embodiments, the biomolecule conjugate of Formula (VIA) is a biomolecule conjugate of Formula (VIG), Formula (VIH), or Formula (VIJ):

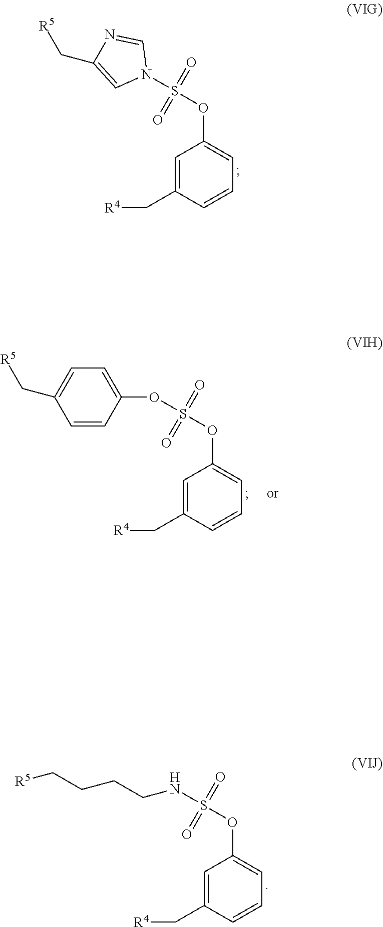

Provided herein are biomolecule conjugates comprising a first biomolecule moiety linked to a second biomolecule moiety by a bioconjugate linker of Formula (IX):

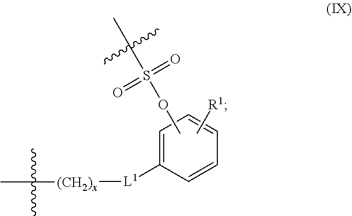

wherein R¹, L¹, and x are as defined herein. In embodiments, R¹ is an electron-withdrawing group. In embodiments, —OS(═O)₂F is ortho to the carbon atom linked to L¹. In embodiments, —OS(═O)₂F is meta to the carbon atom linked to L¹. In embodiments, —OS(═O)₂F is para to the carbon atom linked to L¹. In embodiments, R′ is ortho to —OS(═O)₂F. In embodiments, R¹ is meta to —OS(═O)₂F. In embodiments, R¹ is para to —OS(═O)₂F.

Provided herein are biomolecule conjugates of Formula (IXA):

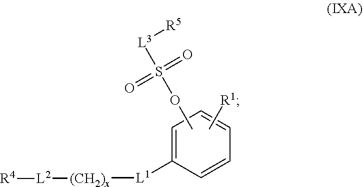

wherein R¹, R⁴, R⁵, L¹, L², L¹, and x are as defined herein. In embodiments, R¹ is an electron-withdrawing group. In embodiments, —OS(═O)₂F is ortho to the carbon atom linked to L¹. In embodiments, —OS(═O)₂F is meta to the carbon atom linked to L¹. In embodiments, —OS(═O)₂F is para to the carbon atom linked to L¹. In embodiments, R¹ is ortho to —OS(═O)₂F. In embodiments, R¹ is meta to —OS(═O)₂F. In embodiments, R¹ is para to —OS(═O)₂F.

Provided herein are biomolecule conjugates of Formula (IXB):

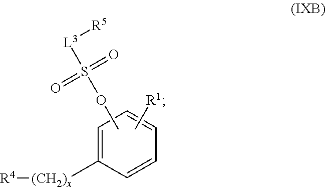

wherein R¹, R⁴, R⁵, L³, and x are as defined herein. In embodiments, R¹ is an electron-withdrawing group. In embodiments, —OS(═O)₂F is ortho to the carbon atom linked to L¹. In embodiments, —OS(═O)₂F is meta to the carbon atom linked to L¹. In embodiments, —OS(═O)₂F is para to the carbon atom linked to L¹. In embodiments, R¹ is ortho to —OS(═O)₂F. In embodiments, R¹ is meta to —OS(═O)₂F. In embodiments, R¹ is para to —OS(═O)₂F.

Provided herein are biomolecule conjugates of Formula (IXC):

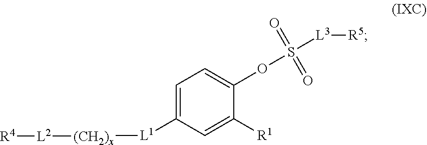

wherein R¹, R⁴, R⁵, L¹, L², L³, and x are as defined herein.

Provided herein are biomolecule conjugates of Formula (IXD):

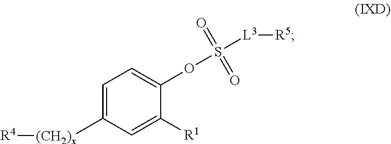

wherein R¹, R⁴, R⁵, L³, and x are as defined herein.

Provided herein are biomolecule conjugates of Formula (IXE):

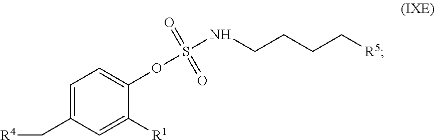

wherein R¹, R⁴, and R⁵ are as defined herein.

Provided herein are biomolecule conjugates of Formula (IXF):

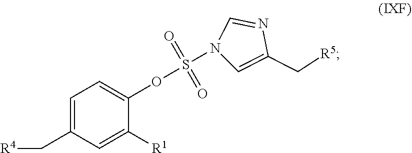

wherein R¹, R⁴, and R⁵ are as defined herein.

Provided herein are biomolecule conjugates of Formula (IXG):

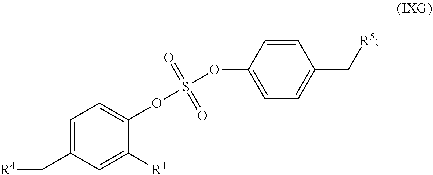

wherein R¹, R⁴, and R⁵ are as defined herein.

Provided herein are proteins having the structure of Formula (X) or Formula (XA):

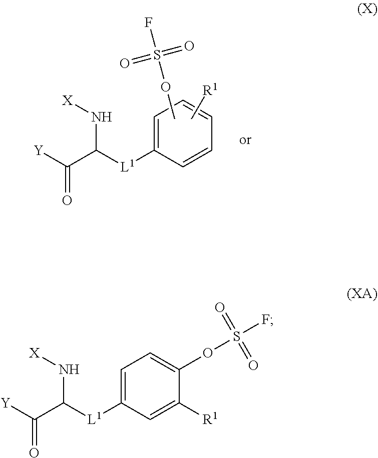

wherein X is —H, a peptidyl moiety, or an amino acid moiety; Y is —OH, a peptidyl moiety, or an amino acid moiety; and R¹ and L¹ are as defined herein. In embodiments, X is —H, Y is a peptidyl moiety, and R¹ and L¹ are as defined herein. In embodiments, X is a peptidyl moiety, Y is —OH, and R¹ and L¹ are as defined herein. In embodiments, X is a peptidyl moiety, Y is a peptidyl moiety, and R¹ and L¹ are as defined herein. In embodiments, (i) X is a peptidyl moiety and Y is OH; (ii) Y is a peptidyl moiety and X is H; or (iii) X and Y are each independently a peptidyl moiety; wherein and R¹ and L¹ are as defined herein. In embodiments, R¹ is an electron-withdrawing group. In embodiments, L¹ is —CH₃— and R¹ is fluorine.

Provided herein are proteins having the structure of Formula (XI) or Formula (XIA):

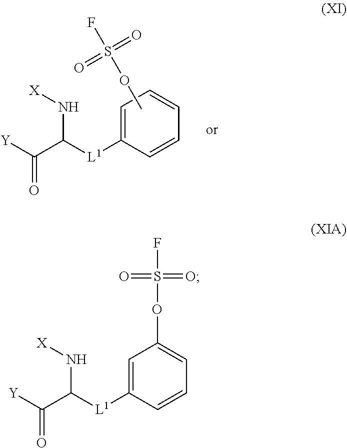

wherein X is —H, a peptidyl moiety, or an amino acid moiety; Y is —OH, a peptidyl moiety, or an amino acid moiety; and L¹ is as defined herein. In embodiments, X is —H, Y is a peptidyl moiety, and L¹ is as defined herein. In embodiments, X is a peptidyl moiety, Y is —OH, and L¹ is as defined herein. In embodiments, X is a peptidyl moiety, Y is a peptidyl moiety, and L¹ is as defined herein. In embodiments, (i) X is a peptidyl moiety and Y is OH; (ii) Y is a peptidyl moiety and X is H; or (iii) X and Y are each independently a peptidyl moiety; wherein and L¹ is as defined herein. In embodiments, L¹ is —CH₃—.

### Substituents

With reference to the compounds described herein, x is an integer from 0 to 8. In embodiments, x is an integer from 1 to 8. In embodiments, x is an integer from 1 to 7. In embodiments, x is an integer from 1 to 6. In embodiments, x is an integer from 1 to 5. In embodiments, x is an integer from 1 to 4. In embodiments, x is an integer from 1 to 3. In embodiments, x is an integer of 1 or 2. In embodiments, x is 1. In embodiments, x is 2. In embodiments, x is 3. In embodiments, x is 4. In embodiments, x is 5. In embodiments, x is 6. In embodiments, x is 7. In embodiments, x is 8. In embodiments, x is 0.

With reference to the compounds described herein, R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —NR₃⁺, substituted or unsubstituted alkyl, substituted or unsubstituted heteroalkyl, substituted or unsubstituted aryl, or substituted or unsubstituted heteroaryl. In embodiments, R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —NR₃⁺, substituted or unsubstituted alkyl, or substituted or unsubstituted heteroalkyl. In embodiments, R¹ is halogen, —CX¹₃, —CHX¹₂, —CH₂X¹, —OCX¹₃, —OCH₂X¹, —OCHX¹₂, —CN, —SO_{n1R}^{1A}, —SOᵥ₁NR^{1A}R^{1B}, —NHC(O)NR^{1A}R^{1B}, N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —NR₃⁺, substituted or unsubstituted alkyl, or substituted or unsubstituted heteroalkyl.

In embodiments, R¹ is an electron-donating group or an electron-withdrawing group.

In embodiments, R¹ is an electron-withdrawing group. In embodiments, the electron-withdrawing group is halogen, —CX¹₃, —CHX¹₂, —CH₂X¹, —CN, —SOₙ₁R^{1A}, —SOᵥ₁NR^{1A}R^{1B}, —N(O)ₘ₁, —C(O)R^{1A}, —C(O)OR^{1A}, —C(O)NR^{1A}R^{1B}, —NR^{1A}OR^{1B}, —NR₃⁺, substituted or unsubstituted alkyl, or substituted or unsubstituted heteroalkyl; wherein X¹, R^{1A}, R^{1B}, n1, v1, and m1 are as defined herein. In embodiments, R^{1A} and R^{1B} are hydrogen.

In embodiments, R¹ is an electron-donating group. In embodiments, the electron-donating group is —Cl, —Br, —I, —CX²³, —CHX²², —OCX¹₃, —OCH₂X¹, —OCHX¹₂, —OCOR^{1A}, —OC(O)R^{1A}, —OC(O)NR^{1A}R^{1B}, —SR^{1A}, —PR^{1A}R^{1B}, —NHC(O)NR^{1A}R^{1B}, —NR^{1A}R^{1B}, —OR^{1A}, —NR^{1A}SO₂R^{1B}, NR^{1A}C(O)R^{1B}, —NR^{1A}C(O)OR^{1B}, substituted or unsubstituted alkyl, substituted or unsubstituted heteroalkyl, substituted or unsubstituted aryl, or substituted or unsubstituted heteroaryl. In embodiments, the substituted or unsubstituted alkyl is substituted or unsubstituted alkene. In embodiments, the electron-donating group is unsubstituted alkene. In embodiments, the substituted or unsubstituted alkyl is substituted or unsubstituted alkyne. In embodiments, R^{1A} and R^{1B} are hydrogen. In embodiments, the electron-donating group is unsubstituted alkyne.

In embodiments of the compounds described herein, R¹ is substituted or unsubstituted heteroalkyl. In embodiments, R¹ is unsubstituted heteroalkyl. In embodiments, R¹ is unsubstituted 2 to 8 membered heteroalkyl. In embodiments, R¹ is unsubstituted 2 to 6 membered heteroalkyl. In embodiments, R¹ is unsubstituted 2 to 4 membered heteroalkyl. In embodiments, R¹ is —O(CH₂)ₘCH₃, and m is an integer from 0 to 6. In embodiments, R¹ is —O(CH₂)ₘCH₃, and m is an integer from 0 to 4. In embodiments, R¹ is —O(CH₂)ₘCH₃, and m is an integer from 0 to 3. In embodiments, R¹ is —O(CH₂)ₘCH₃, and m is an integer from 0 to 2. In embodiments, R¹ is —O(CH₂)ₘCH₃, and m is 0 or 1. In embodiments, R¹ is —OCH₃. In embodiments, R¹ is —OCH₂CH₃, In embodiments, R¹ is —O(CH₂)₂CH₃, In embodiments, R¹ is —O(CH₂)₃CH₃. In embodiments, R¹ is hydrogen.

In embodiments of the compounds described herein, R¹ is halogen. In embodiments, R¹ is fluorine, chlorine, bromine, or iodine. In embodiments, R¹ is fluorine, chlorine, or bromine. In embodiments, R¹ is fluorine or chlorine. In embodiments, R¹ is fluorine or bromine. In embodiments, R¹ is chlorine or bromine. In embodiments, R¹ is fluorine. In embodiments, R¹ is chlorine. In embodiments, R¹ is bromine. In embodiments, R¹ is iodine.

In embodiments, R¹ is —CX¹₃, —CHX¹₂, or —CH₂X¹, wherein X¹ is halogen. In embodiments, R¹ is —CH₂X¹. In embodiments, R¹ is —CHX¹₂. In embodiments, R¹ is —CX¹. In embodiments, R¹ is —CF₃. In embodiments, R¹ is —CHF₂. In embodiments, R¹ is —CH₂F. In embodiments, R¹ is —CCl₃. In embodiments, R¹ is —CHCl₂. In embodiments, R¹ is —CH₂Cl. In embodiments, R¹ is —CBr₃. In embodiments, R¹ is —CHBr₂. In embodiments, R¹ is —CH₂Br. In embodiments, R¹ is —CN. In embodiments, R¹ is —N(O)ₘ₁. In embodiments, R¹ is —NO₂. In embodiments, R¹ is —SOₙ₁R^{1A}. In embodiments, R¹ is —SO₂H. In embodiments, R¹ is —SOᵥ₁NR^{1A}R^{1B}. In embodiments, R¹ is —SO₂NH₂. In embodiments, R¹ is —NR₃⁺.

In embodiments of the compounds described herein, R¹ is an alkyl group substituted with an electron-withdrawing group. In embodiments, R¹ is a halogen-substituted alkyl group. In embodiments, —(CH₂)_{w}CX¹₃, —(CH₂)_{w}CHX¹₂, or —(CH₂)_{w}CH₂X¹, wherein w is an integer from 1 to 5, and X¹ is halogen. In embodiments, w is 1. In embodiments, w is 2. In embodiments, w is 3. In embodiments, w is 4. In embodiments, w is 5.

With reference to the compounds described herein, R¹ is ortho, para, or meta to the —O—S(═O)₂F group. In embodiments, R¹ is ortho to the —O—S(═O)₂F group. In embodiments, R¹ is para to the —O—S(═O)₂F group. In embodiments, R¹ is meta to the —O—S(═O)₂F group.

With reference to the compounds described herein, R¹ is ortho, para, or meta to the —S(═O)₂F group. In embodiments, R¹ is ortho to the —S(═O)₂F group. In embodiments, R¹ is para to the —S(═O)₂F group. In embodiments, R¹ is meta to the —S(═O)₂F group.

With reference to the compounds described herein, R^{1A} is hydrogen, substituted or unsubstituted alkyl, or substituted or unsubstituted heteroalkyl. In embodiments, R^{1A} is hydrogen, unsubstituted alkyl, or unsubstituted heteroalkyl. In embodiments, R^{1A} is hydrogen, substituted or unsubstituted C₁₋₄ alkyl, or substituted or unsubstituted 2 to 4 membered heteroalkyl. In embodiments, R^{1A} is hydrogen, unsubstituted C₁₋₄ alkyl, or unsubstituted 2 to 4 membered heteroalkyl. In embodiments, R^{1A} is hydrogen. In embodiments, R^{1A} is unsubstituted C₁₋₄ alkyl. In embodiments, R′^{A} is unsubstituted 2 to 4 membered heteroalkyl. In embodiments, R^{1A} is hydrogen and R^{1B} is hydrogen.

With reference to the compounds described herein, R^{1B} is hydrogen, substituted or unsubstituted alkyl, or substituted or unsubstituted heteroalkyl. In embodiments, R^{1B} is hydrogen, unsubstituted alkyl, or unsubstituted heteroalkyl. In embodiments, R^{1B} is hydrogen, substituted or unsubstituted C₁₋₄ alkyl, or substituted or unsubstituted 2 to 4 membered heteroalkyl. In embodiments, R^{1B} is hydrogen, unsubstituted C₁₋₄ alkyl, or unsubstituted 2 to 4 membered heteroalkyl. In embodiments, R^{1B} is hydrogen. In embodiments, R^{1B} is unsubstituted C₁₋₄ alkyl. In embodiments, R^{1B} is unsubstituted 2 to 4 membered heteroalkyl. In embodiments, R^{1A} is hydrogen and R^{1B} is hydrogen.

With reference to the compounds described herein, X¹ is independently —F, —Cl, —Br, or —I. In embodiments, X¹ is independently —F, —Cl, or —Br. In embodiments, X¹ is independently —F or —Cl. In embodiments, X¹ is —F. In embodiments, X¹ is —Cl. In embodiments, X¹ is —Br. In embodiments, X¹ is —I.

With reference to the compounds described herein, n1 is an integer from 0 to 4. In embodiments n1 is an integer from 0 to 3. In embodiments n1 is an integer from 0 to 2. In embodiments n1 is 0. In embodiments n1 is 1. In embodiments n1 is 2. In embodiments n1 is 3. In embodiments n1 is 4.

With reference to the compounds described herein, m1 is 1 or 2. In embodiments, m1 is 1. In embodiments, m1 is 2.

With reference to the compounds described herein, v1 is 1 or 2. In embodiments, v1 is 1. In embodiments, v1 is 2.

With reference to the compounds described herein, L¹ is a bond, substituted or unsubstituted alkylene, or substituted or unsubstituted heteroalkylene. In embodiments, L¹ is a bond. In embodiments, L¹ is substituted or unsubstituted alkylene. In embodiments, L¹ is substituted or unsubstituted C₁₋₆ alkylene. In embodiments, L¹ is substituted or unsubstituted C₁₋₄ alkylene. In embodiments, L¹ is unsubstituted alkylene. In embodiments, L¹ is unsubstituted C₁₋₆ alkylene. In embodiments, L¹ is unsubstituted C₁₋₄ alkylene. In embodiments, L¹ is methylene. In embodiments, L¹ is ethylene. In embodiments, L¹ is propylene. In embodiments, L¹ is substituted or unsubstituted heteroalkylene. In embodiments, L¹ is substituted or unsubstituted 2 to 8 membered heteroalkylene. In embodiments, L¹ is substituted or unsubstituted 2 to 6 membered heteroalkylene. In embodiments, L¹ is —NH—C(O)—(CH₂)_{y}— or —NH—C(O)—O—(CH₂)_{y}—, and y is an integer from 0 to 6. In embodiments, L¹ is —NH—C(O)—(CH₂)_{y}— or —NH—C(O)—O—(CH₂)_{y}—, and y is an integer from 0 to 5. In embodiments, L¹ is —NH—C(O)—(CH₂)_{y}— or —NH—C(O)—O—(CH₂)_{y}—, and y is an integer from 0 to 4. In embodiments, L¹ is —NH—C(O)—(CH₂)_{y}— or —NH—C(O)—O—(CH₂)_{y}—, and y is an integer from 0 to 3. In embodiments, L¹ is —NH—C(O)—(CH₂)_{y}— or —NH—C(O)—O—(CH₂)_{y}—, and y is an integer from 0 to 2. In embodiments, L¹ is —NH—C(O)—(CH₂)_{y}—, and y is an integer from 0 to 3. In embodiments, L¹ is —NH—C(O)—. In embodiments, L¹ is —NH—C(O)—(CH₂)— In embodiments, L¹ is —NH—C(O)—(CH₂)₂—. In embodiments, L¹ is —NH—C(O)—(CH₂)₃—. In embodiments, L¹ is —NH—C(O)—O—(CH₂)_{y}—, and y is an integer from 0 to 3. In embodiments, L¹ is —NH—C(O)—O—. In embodiments, L¹ is —NH—C(O)—O—(CH₂)—. In embodiments, L¹ is —NH—C(O)—O—(CH₂)₂—. In embodiments, L¹ is —NH—C(O)—O—(CH₂)₃—.

With reference to the compounds described herein, L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-substituted or unsubstituted heteroarylene. In embodiments, L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, unsubstituted alkylene, unsubstituted heteroalkylene, unsubstituted cycloalkylene, unsubstituted heterocycloalkylene, unsubstituted arylene, or unsubstituted heteroarylene. In embodiments, L² is a bond. In embodiments, the alkylene is a C₁₋₆ alkylene. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

In embodiments of the compounds described herein, L¹ is a bond and L² is a bond. In embodiments of the compounds described herein, R² is a peptidyl moiety, R³ is a peptidyl moiety, L¹ is a bond, and L² is a bond.

With reference to the compounds described herein, R^{2A} and R^{2B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene. In embodiments, R^{2A} and R^{2B} are hydrogen.

With reference to the compounds described herein, L¹² is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹³-substituted or unsubstituted heteroarylene. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, RA and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, L¹³ is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

In embodiments of the compounds described herein, the peptidyl moiety of R⁴ comprises an antibody or an antibody variant; and the peptidyl moiety of R⁵ comprises a receptor protein. In embodiments, the peptidyl moiety of R⁴ comprises an antibody or an antibody variant; and the peptidyl moiety of R⁵ comprises a receptor protein, wherein the receptor protein comprises a lysine, histidine, or tyrosine bonded to L³, where L³ is a bond. In embodiments, R⁴ comprises an antibody. In embodiments, R⁴ comprises an antibody variant. In embodiments, the antibody variant is a variant as defined herein. In embodiments, the antibody variant is a single-chain variable fragment, a single-domain antibody, an affibody, or an antigen-binding fragment. In embodiments, the antibody variant is a single-chain variable fragment. In embodiments, the antibody variant is a single-domain antibody. In embodiments, the antibody variant is an affibody. In embodiments, the antibody variant is an antigen-binding fragment. In embodiments, the receptor protein is any receptor protein described herein.

In embodiments of the compounds described herein, the peptidyl moiety of R⁴ comprises a receptor protein; and the peptidyl moiety of R⁵ comprises an antibody or an antibody variant. In embodiments, the peptidyl moiety of R⁴ comprises a receptor protein; and the peptidyl moiety of R⁵ comprises an antibody or an antibody variant; wherein the antibody or antibody variant comprises a lysine, histidine, or tyrosine bonded to L³, where L³ is a bond. In embodiments, R⁵ comprises an antibody. In embodiments, R⁵ comprises an antibody variant. In embodiments, the antibody variant is a variant as defined herein. In embodiments, the antibody variant is a single-chain variable fragment, a single-domain antibody, an affibody, or an antigen-binding fragment. In embodiments, the antibody variant is a single-chain variable fragment. In embodiments, the antibody variant is a single-domain antibody. In embodiments, the antibody variant is an affibody. In embodiments, the antibody variant is an antigen-binding fragment. In embodiments, the receptor protein is any receptor protein described herein.

In embodiments of the compounds described herein, R⁵ is a peptidyl moiety comprising a lysine, histidine, or tyrosine bonded to L³. In embodiments, R⁵ is a peptidyl moiety comprising a lysine bonded to L³. In embodiments, R⁵ is a peptidyl moiety comprising a histidine bonded to L³. In embodiments, R⁵ is a peptidyl moiety comprising a tyrosine bonded to L³. In embodiments, R⁵ is a peptidyl moiety comprising a lysine, histidine, or tyrosine bonded to L³, where L³ is a bond. In embodiments, R⁵ is a peptidyl moiety comprising a lysine bonded to L³, where L³ is a bond. In embodiments, R⁵ is a peptidyl moiety comprising a histidine bonded to L³, where L³ is a bond. In embodiments, R⁵ is a peptidyl moiety comprising a tyrosine bonded to L³, where L³ is a bond. In embodiments, L² is a bond.

In embodiments, the biomolecules, proteins, and peptidyl moieties described herein comprise a receptor protein. In embodiments, the receptor protein is a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, or a combination of two or more thereof. In embodiments, the receptor protein is an integrin. In embodiments, the receptor protein is a somatostain receptor. In embodiments, the receptor protein is a gonadotropin-releasing hormone receptor. In embodiments, the receptor protein is a bombesin receptor. In embodiments, the receptor protein is a vasoactive intestinal peptide receptor. In embodiments, the receptor protein is a neurotensin receptor. In embodiments, the receptor protein is a cholecystokinin 2 receptor. In embodiments, the receptor protein is a melanocortin receptor. In embodiments, the receptor protein is a ghrelin receptor.

In embodiments, the receptor protein is a receptor expressed on a cancer cell. In embodiments, the receptor protein is a receptor overexpressed on a cancer cell relative to a control.

In embodiments, the receptor protein is a G protein-coupled receptor. In embodiments, the receptor protein is a receptor tyrosine kinase. In embodiments, the receptor protein is a an ErbB receptor. In embodiments, the receptor protein is an epidermal growth factor receptor (EGFR). In embodiments, the receptor protein is epidermal growth factor receptor 1 (HER1). In embodiments, the receptor protein is epidermal growth factor receptor 2 (HER2). In embodiments, the receptor protein is epidermal growth factor receptor 3 (HER3). In embodiments, the receptor protein is epidermal growth factor receptor 4 (HER4).

### Proteins

Provided herein are proteins comprising an unnatural amino acid within CDR-L1, CDR-L2, CDR-L3, CDR-H1, CDR-H2, or CDR-H3, wherein the protein is an antigen-binding fragment, a single-chain variable fragment, or an antibody. In embodiments, the protein is an antigen-binding fragment. In embodiments, the protein is a single-chain variable fragment. In embodiments, the protein is an antibody. In embodiments, the protein has one unnatural amino acid within CDR-L1. In embodiments, the protein has one unnatural amino acid within CDR-L2. In embodiments, the protein has one unnatural amino acid within CDR-L3. In embodiments, the protein has one unnatural amino acid within CDR-H1. In embodiments, the protein has one unnatural amino acid within CDR-H2. In embodiments, the protein has one unnatural amino acid within CDR-H3. In embodiments, the protein has two or more unnatural amino acids within CDR-L1, CDR-L2, CDR-L3, CDR-H1, CDR-H2, or CDR-H3. The two or more unnatural acids can be in the same or different CDR, and can be in the same or different chain (i.e., light or heavy).

Provided herein are Fabs comprising an unnatural amino acid. Provided herein are Fabs comprising an unnatural amino acid, wherein the unnatural amino acid is FSK. Provided herein are Fabs comprising an unnatural amino acid, wherein the unnatural amino acid is FSY. Provided herein are Fabs comprising an unnatural amino acid, wherein the unnatural amino acid is meta-FSY. Provided herein are Fabs comprising an unnatural amino acid, wherein the unnatural amino acid is FFY. Provided herein are Fabs comprising an unnatural amino acid, wherein the unnatural amino acid comprises a side chain of Formula (II), Formula (V), or Formula (VIII). Provided herein are Fabs comprising an unnatural amino acid, wherein the unnatural amino acid comprises a side chain of Formula (IIC). Provided herein are Fabs comprising an unnatural amino acid, wherein the unnatural amino acid comprises a side chain of Formula (IIE). Provided herein are Fabs comprising an unnatural amino acid, wherein the unnatural amino acid comprises a side chain of Formula (VA). Provided herein are Fabs comprising an unnatural amino acid, wherein the unnatural amino acid comprises a side chain of Formula (VB). Provided herein are Fabs comprising an unnatural amino acid, wherein the unnatural amino acid comprises a side chain of Formula (VIIIC).

In embodiments, the Fab is trastuzumab Fab. In embodiments, trastuzumab Fab comprises CDR-L1 as set forth in SEQ ID NO:163, CDR-L2 as set forth in SEQ ID NO:164, CDR-L3 as set forth in SEQ ID NO:165, CDR-H1 as set forth in SEQ ID NO:171, CDR-H2 as set forth in SEQ ID NO: 172, and CDR-H3 as set forth in SEQ ID NO:173. In embodiments, trastuzumab Fab comprises the unnatural amino acid at a position corresponding to position 92 of the light chain. In embodiments, trastuzumab Fab comprises the unnatural amino acid at a position corresponding to position 50 of the light chain. In embodiments, the unnatural amino acid is FSY. In embodiments, the unnatural amino acid is FSK. In embodiments, the unnatural amino acid is FFY. In embodiments, the unnatural amino acid is meta-FSY. In embodiments, the unnatural amino acid is FSK. In embodiments, the unnatural amino acid is meta-FSK. In embodiments, the unnatural amino acid comprises a side chain of Formula (V). In embodiments, the unnatural amino acid comprises a side chain of Formula (VA). In embodiments, the unnatural amino acid comprises a side chain of Formula (VB). In embodiments, trastuzumab Fab comprises an unnatural amino acid having side chain of Formula (V) covalently bonded to HER2. In embodiments, trastuzumab Fab comprising the unnatural amino acid having a side chain of Formula (V) is covalently bonded to a lysine, histidine, or tyrosine on HER2. In embodiments, trastuzumab Fab comprising the unnatural amino acid having the side chain of Formula (V) is covalently bonded to a lysine at a position corresponding to position 593 on HER2. In embodiments, the unnatural amino acid comprises a side chain of Formula (VIII) or embodiments thereof. In embodiments, the disclosure provides a biomolecule conjugate comprising trastuzumab Fab as described herein, including embodiments thereof, covalently bonded to HER2.

In embodiments, trastuzumab Fab comprises CDR-L1 as set forth in SEQ ID NO:163, CDR-L2 as set forth in SEQ ID NIO:164, CDR-L3 as set forth in SEQ ID NO:166, CDR-H1 as set forth in SEQ ID NO:171, CDR-H2 as set forth in SEQ ID NO:172, and CDR-H3 as set forth in SEQ ID NO:173. In embodiments, trastuzumab Fab comprises CDR-L1 as set forth in SEQ ID NO:163, CDR-L2 as set forth in SEQ ID NIO:164, CDR-L3 as set forth in SEQ ID NO:167, CDR-H1 as set forth in SEQ ID NO:171, CDR-H2 as set forth in SEQ ID NO:172, and CDR-H3 as set forth in SEQ ID NO:173. In embodiments, trastuzumab Fab light chain has at least 90% sequence identity to SEQ ID NO:168, and trastuzumab Fab heavy chain has at least 90% sequence identity to SEQ ID NO:170, provided that the light chain and heavy chain have 100% sequence identity to the CDRs therein. In embodiments, trastuzumab Fab light chain has at least 92% sequence identity to SEQ ID NO:168, and trastuzumab Fab heavy chain has at least 92% sequence identity to SEQ ID NO:170, provided that the light chain and heavy chain have 100% sequence identity to the CDRs therein. In embodiments, trastuzumab Fab light chain has at least 94% sequence identity to SEQ ID NO:168, and trastuzumab Fab heavy chain has at least 94% sequence identity to SEQ ID NO:170, provided that the light chain and heavy chain have 100% sequence identity to the CDRs therein. In embodiments, trastuzumab Fab light chain has at least 95% sequence identity to SEQ ID NO:168, and trastuzumab Fab heavy chain has at least 96% sequence identity to SEQ ID NO:170, provided that the light chain and heavy chain have 100% sequence identity to the CDRs therein. In embodiments, trastuzumab Fab light chain has at least 96% sequence identity to SEQ ID NO:168, and trastuzumab Fab heavy chain has at least 96% sequence identity to SEQ ID NO: 170, provided that the light chain and heavy chain have 100% sequence identity to the CDRs therein. In embodiments, trastuzumab Fab light chain has at least 98% sequence identity to SEQ ID NO:168, and trastuzumab Fab heavy chain has at least 98% sequence identity to SEQ ID NO:170, provided that the light chain and heavy chain have 100% sequence identity to the CDRs therein. In embodiments, trastuzumab Fab light chain comprises SEQ ID NO:168, and trastuzumab Fab heavy chain comprises SEQ ID NO:170. In embodiments, trastuzumab Fab light chain has at least 90% sequence identity to SEQ ID NO:169, and trastuzumab Fab heavy chain has at least 90% sequence identity to SEQ ID NO:170, provided that the light chain and heavy chain have 100% sequence identity to the CDRs therein. In embodiments, trastuzumab Fab light chain has at least 92% sequence identity to SEQ ID NO:169, and trastuzumab Fab heavy chain has at least 92% sequence identity to SEQ ID NO:170, provided that the light chain and heavy chain have 100% sequence identity to the CDRs therein. In embodiments, trastuzumab Fab light chain has at least 94% sequence identity to SEQ ID NO:169, and trastuzumab Fab heavy chain has at least 94% sequence identity to SEQ ID NO:170, provided that the light chain and heavy chain have 100% sequence identity to the CDRs therein. In embodiments, trastuzumab Fab light chain has at least 95% sequence identity to SEQ ID NO:169, and trastuzumab Fab heavy chain has at least 95% sequence identity to SEQ ID NO:170, provided that the light chain and heavy chain have 100% sequence identity to the CDRs therein. In embodiments, trastuzumab Fab light chain has at least 96% sequence identity to SEQ ID NO:169, and trastuzumab Fab heavy chain has at least 96% sequence identity to SEQ ID NO:170, provided that the light chain and heavy chain have 100% sequence identity to the CDRs therein. In embodiments, trastuzumab Fab light chain has at least 98% sequence identity to SEQ ID NO:169, and trastuzumab Fab heavy chain has at least 98% sequence identity to SEQ ID NO:170, provided that the light chain and heavy chain have 100% sequence identity to the CDRs therein. In embodiments, trastuzumab Fab light chain comprises SEQ ID NO:169, and trastuzumab Fab heavy chain comprises SEQ ID NO:170. In embodiments, the disclosure provides a biomolecule conjugate comprising trastuzumab Fab as described herein, including embodiments thereof, covalently bonded to HER2.

### Nanobodies

Provided herein are nanobodies comprising an unnatural amino acid. Provided herein are single-domain antibodies having an unnatural amino acid side chain; wherein the unnatural amino acid side chain is capable of covalently binding to lysine, tyrosine, or histidine. In aspects, the unnatural amino acid side chain is capable of covalently binding to lysine or tyrosine. In aspects, the unnatural amino acid side chain is capable of covalently binding to lysine. In aspects, the unnatural amino acid side chain is capable of covalently binding to tyrosine. Provided herein are nanobodies comprising an unnatural amino acid, wherein the unnatural amino acid is within CDR1, CDR2, or CDR3 of the nanobody. Provided herein are nanobodies comprising one unnatural amino acid, wherein the one unnatural amino acid is within CDR1, CDR2, or CDR3 of the nanobody. Provided herein are nanobodies comprising two unnatural amino acids, wherein the two unnatural amino acids are within CDR1, CDR2, or CDR3 of the nanobody. Provided herein are nanobodies comprising three unnatural amino acids, wherein the three unnatural amino acids are within CDR1, CDR2, or CDR3 of the nanobody. Provided herein are nanobodies comprising four unnatural amino acids, wherein the four unnatural amino acids are within CDR1, CDR2, or CDR3 of the nanobody. Provided herein are nanobodies comprising an unnatural amino acid, wherein the unnatural amino acid is within CDR1 of the nanobody. Provided herein are nanobodies comprising an unnatural amino acid, wherein the unnatural amino acid is within CDR1, but not within CDR2 or CDR3 of the nanobody. Provided herein are nanobodies comprising one unnatural amino acid, wherein the one unnatural amino acid is within CDR1 of the nanobody. Provided herein are nanobodies comprising an unnatural amino acid, wherein the unnatural amino acid is within CDR2 of the nanobody. Provided herein are nanobodies comprising an unnatural amino acid, wherein the unnatural amino acid is within CDR2, and there are not any unnatural amino acids within CDR1 or CDR3 of the nanobody. Provided herein are nanobodies comprising one unnatural amino acid, wherein the one unnatural amino acid is within CDR2 of the nanobody. Provided herein are nanobodies comprising an unnatural amino acid, wherein the unnatural amino acid is within CDR3 of the nanobody. Provided herein are nanobodies comprising an unnatural amino acid, wherein the unnatural amino acid is within CDR3, and there are not any unnatural amino acids within CDR1 or CDR2 of the nanobody. Provided herein are nanobodies comprising one unnatural amino acid, wherein the one unnatural amino acid is within CDR3 of the nanobody. In embodiments, the unnatural amino acid is FSK. In embodiments, the unnatural amino acid is FSY. In embodiments, the unnatural amino acid is meta-FSY. In embodiments, the unnatural amino acid is FFY. In embodiments, the unnatural amino acid comprises a side chain of Formula (II). In embodiments, the unnatural amino acid comprises a side chain of Formula (V). In embodiments, the unnatural amino acid comprises a side chain of Formula (VIII). In embodiments, the unnatural amino acid comprises a side chain of Formula (IIC). Provided herein are nanobodies comprising an unnatural amino acid, wherein the unnatural amino acid comprises a side chain of Formula (IIE). In embodiments, the unnatural amino acid comprises a side chain of Formula (VA). Provided herein are nanobodies comprising an unnatural amino acid, wherein the unnatural amino acid comprises a side chain of Formula (VB). In embodiments, the unnatural amino acid comprises a side chain of Formula (VIIIC).

Provided herein nanobodies comprising an unnatural amino acid within CDR1, CDR2, or CDR3 of the nanobody; wherein the unnatural amino acid comprises a side chain of Formula (II):

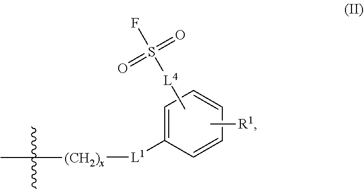

wherein: L⁴ is a bond or —O—; x is an integer from 1 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2. The substituents have the definitions as described herein. In embodiments, the unnatural amino acid comprises a side chain of Formula (IE-A):

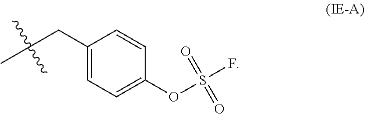 [SMILES: *Cc1ccc(OS(=O)(=O)F)cc1]

In embodiments, the unnatural amino acid comprises a side chain of Formula (VA):

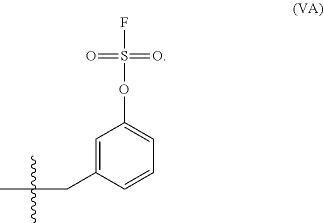 [SMILES: *Cc1cccc(OS(=O)(=O)F)c1]

In embodiments, the unnatural amino acid comprises a side chain of Formula (VIIIC):

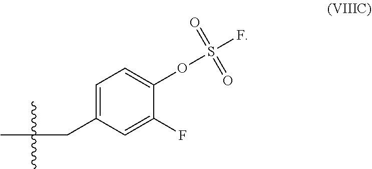 [SMILES: *Cc1ccc(OS(=O)(=O)F)c(F)c1]

In embodiments, the unnatural amino acid comprises a side chain of Formula (VB):

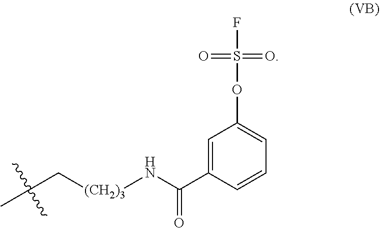

In embodiments, the unnatural amino acid comprises a side chain of Formula (VB):

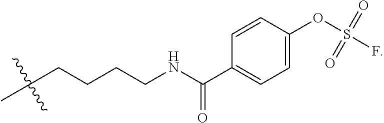 [SMILES: *CCCCNC(=O)c1ccc(OS(=O)(=O)F)cc1]

In embodiments, the nanobody comprising an unnatural amino acid within CDR1, CDR2, or CDR3 of the nanobody is not nanobody 7D12 or nanobody KN035. In embodiments, the nanobody comprising an unnatural amino acid within CDR1, CDR2, or CDR3 of the nanobody has less than 100% sequence identity with CDR1 as set forth in SEQ ID NO:155, CDR2 as set forth in SEQ ID NO:156, or CDR3 as set forth in SEQ ID NO:157. In embodiments, the nanobody having CDR1 as set forth in SEQ ID NO:155, CDR2 as set forth in SEQ ID NO:156, and CDR3 as set forth in SEQ ID NO: 157 does not contain an FSY unnatural amino acid in CDR1, CDR2, or CDR3 and does not contain an FSK unnatural amino acid in CDR1, CDR2, or CDR3. In embodiments, the nanobody comprising an unnatural amino acid within CDR1, CDR2, or CDR3 of the nanobody has less than 100% sequence identity to CDR1, CDR2, or CDR3 in SEQ ID NO:177 or SEQ ID NO:178. In embodiments, the nanobody comprising an unnatural amino acid within CDR1, CDR2, or CDR3 of the nanobody has less than 100% sequence identity to SEQ ID NO:177 or SEQ ID NO:178. In embodiments, the nanobody as set forth in SEQ ID NO:177 or SEQ ID NO:178 does not contain an FSY unnatural amino acid.

Provided herein is nanobody 2rs15d, wherein the nanobody comprises an unnatural amino acid. In embodiments, the nanobody comprises CDR1 as set forth in SEQ ID NO:67, CDR2 as set forth in SEQ ID NO:68; and CDR3 as set forth in SEQ ID NO:69, wherein the nanobody comprises an unnatural amino acid in CDR1, CDR2, or CDR3. In embodiments, the nanobody comprises CDR1 as set forth in SEQ ID NO:67, CDR2 as set forth in SEQ ID NO:68; and CDR3 as set forth in SEQ ID NO:69, wherein the unnatural amino acid is at a position corresponding to position 54 or 102 in SEQ ID NO:69. In embodiments, the nanobody comprises CDR1 as set forth in SEQ ID NO:67, CDR2 as set forth in SEQ ID NO:68; and CDR3 as set forth in SEQ ID NO: 69. In embodiments, the nanobody comprises CDR1 as set forth in SEQ ID NO:67, CDR2 as set forth in SEQ ID NO:68; and CDR3 as set forth in SEQ ID NO:69, wherein the unnatural amino acid is at a position corresponding to position 54 SEQ ID NO:69. In embodiments, the nanobody comprises CDR1 as set forth in SEQ ID NO:67, CDR2 as set forth in SEQ ID NO:68; and CDR3 as set forth in SEQ ID NO:69. In embodiments, the nanobody comprises CDR1 as set forth in SEQ ID NO:67, CDR2 as set forth in SEQ ID NO:68; and CDR3 as set forth in SEQ ID NO:69, wherein the unnatural amino acid is at a position corresponding to position 102 in SEQ ID NO:69. In embodiments, the unnatural amino acid is FSY, metaFSY, FFY, FSK, or meta-FSK. In embodiments, the nanobody comprises CDR1 as set forth in SEQ ID NO:67, CDR2 as set forth in SEQ ID NO:68; and CDR3 as set forth in SEQ ID NO:70. In embodiments, the nanobody comprises CDR1 as set forth in SEQ ID NO:67, CDR2 as set forth in SEQ ID NO:68; and CDR3 as set forth in SEQ ID NO:71. In embodiments, the nanobody further comprises a detectable agent. In embodiments, the nanobody further comprises a radioisotope. In embodiments, the radioisotope is a positron-emitting radioisotope. In embodiments, the positron-emitting radioisotope is ¹¹C, ¹³N, ¹⁵O, ¹⁸F, ⁶⁴Cu, ⁶⁸Ga, ⁷⁸Br, ⁸²Rb, ⁸⁶Y, ⁸⁹Zr, ⁹⁰Y, ²²Na, ²⁶Al, ⁴⁰K, ⁸³Sr, or ¹²⁴I. In embodiments, the positron-emitting radioisotope is ¹²⁴I. In embodiments, the radioisotope is an alpha-emitting radioisotope. In embodiments, the alpha-emitting radioisotope is ²¹¹At, ²²⁷Th, ²²⁵Ac, ²²³Ra, ²¹³Bi, or ²¹²Bi. In embodiments, the alpha-emitting radioisotope is ²¹¹At. In embodiments, the nanobody further comprises a therapeutic agent. In embodiments, the nanobody further comprises a detectable agent and a therapeutic agent. In embodiments, the disclosure provides a biomolecule conjugate comprising nanobody 2rs15d as described herein, including embodiments thereof, covalently bonded to HER2. In embodiments, the disclosure provides a biomolecule conjugate comprising nanobody 2rs15d as described herein, including embodiments thereof, covalently bonded to HER2 expressed on a cancer tumor.

Provided herein is nanobody mNb6, wherein the nanobody comprises an unnatural amino acid. In embodiments, the nanobody comprises CDR1 as set forth in SEQ ID NO:61, CDR2 as set forth in SEQ ID NO:62; and CDR3 as set forth in SEQ ID NO:63, wherein the nanobody comprises an unnatural amino acid in CDR1, CDR2, or CDR3. In embodiments, the nanobody comprises CDR1 as set forth in SEQ ID NO:61, CDR2 as set forth in SEQ ID NO:62; and CDR3 as set forth in SEQ ID NO:63; wherein the unnatural amino acid is at a position corresponding to position 10 in SEQ ID NO: 63. In embodiments, the nanobody comprises CDR1 as set forth in SEQ ID NO: 61, CDR2 as set forth in SEQ ID NO:62; and CDR3 as set forth in SEQ ID NO:63; wherein the unnatural amino acid is at a position corresponding to position 8 in SEQ ID NO: 63. In embodiments, the nanobody comprises CDR1 as set forth in SEQ ID NO:61, CDR2 as set forth in SEQ ID NO:62; and CDR3 as set forth in SEQ ID NO:63; wherein the unnatural amino acid is at a position corresponding to position 6 in SEQ ID NO:63. In embodiments, the nanobody comprises CDR1 as set forth in SEQ ID NO:61, CDR2 as set forth in SEQ ID NO:62; and CDR3 as set forth in SEQ ID NO:63; wherein the unnatural amino acid is at a position corresponding to position 4 in SEQ ID NO:63. In embodiments, the unnatural amino acid is FSY, metaFSY, FFY, FSK, or meta-FSK. In embodiments, the nanobody comprises CDR1 as set forth in SEQ ID NO:61, CDR2 as set forth in SEQ ID NO:62; and CDR3 as set forth in SEQ ID NO:64, 200, 202, 204, 206, 208, 210, or 212. In embodiments, the nanobody comprises CDR1 as set forth in SEQ ID NO:61, CDR2 as set forth in SEQ ID NO:62; and CDR3 as set forth in SEQ ID NO:64. In embodiments, the nanobody comprises CDR1 as set forth in SEQ ID NO:61, CDR2 as set forth in SEQ ID NO:62; and CDR3 as set forth in SEQ ID NO:200. In embodiments, the nanobody comprises CDR1 as set forth in SEQ ID NO:61, CDR2 as set forth in SEQ ID NO:62; and CDR3 as set forth in SEQ ID NO:202. In embodiments, the nanobody comprises CDR1 as set forth in SEQ ID NO:61, CDR2 as set forth in SEQ ID NO:62; and CDR3 as set forth in SEQ ID NO:204. In embodiments, the nanobody comprises CDR1 as set forth in SEQ ID NO:61, CDR2 as set forth in SEQ ID NO:62; and CDR3 as set forth in SEQ ID NO:206. In embodiments, the nanobody comprises CDR1 as set forth in SEQ ID NO:61, CDR2 as set forth in SEQ ID NO:62; and CDR3 as set forth in SEQ ID NO:208. In embodiments, the nanobody comprises CDR1 as set forth in SEQ ID NO:61, CDR2 as set forth in SEQ ID NO:62; and CDR3 as set forth in SEQ ID NO:210. In embodiments, the nanobody comprises CDR1 as set forth in SEQ ID NO:61, CDR2 as set forth in SEQ ID NO:62; and CDR3 as set forth in SEQ ID NO:212. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent. In embodiments, the disclosure provides a biomolecule conjugate comprising nanobody mNb6 covalently bonded to a coronavirus. In embodiments, the disclosure provides a biomolecule conjugate comprising nanobody mNb6 covalently bonded to SARS-CoV. In embodiments, the disclosure provides a biomolecule conjugate comprising nanobody mNb6 covalently bonded to SARS-CoV-2. In embodiments, the disclosure provides a method of treating COVID-19 in a patient in need thereof comprising administering to a patient an effective amount of nanobody mNb6 as described herein, including embodiments thereof. In embodiments, the disclosure provides a method of treating a coronavirus infection in a patient in need thereof comprising administering to a patient an effective amount of nanobody mNb6 as described herein, including embodiments thereof. In embodiments, the disclosure provides a method of treating a SARS-CoV-2 infection in a patient in need thereof comprising administering to a patient an effective amount of nanobody mNb6 as described herein, including embodiments thereof.

Provided herein is nanobody C21, wherein the nanobody comprises an unnatural amino acid. In embodiments, the nanobody comprises CDR1 as set forth in SEQ ID NO:75, CDR2 as set forth in SEQ ID NO:76; and CDR3 as set forth in SEQ ID NO:77, wherein the nanobody comprises an unnatural amino acid in CDR1, CDR2, or CDR3. In embodiments, the nanobody comprises CDR1 as set forth in SEQ ID NO:75, CDR2 as set forth in SEQ ID NO:76; and CDR3 as set forth in SEQ ID NO:77, wherein the unnatural amino acid is at a position corresponding to position 6 in SEQ ID NO:75. In embodiments, the unnatural amino acid is FSY, metaFSY, FFY, FSK, or meta-FSK. In embodiments, the nanobody comprises CDR1 as set forth in SEQ ID NO:78, CDR2 as set forth in SEQ ID NO:76, and CDR3 as set forth in SEQ ID NO:77. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

Provided herein is nanobody NB13, wherein the nanobody comprises an unnatural amino acid. In embodiments, the nanobody comprises CDR1 as set forth in SEQ ID NO:81, CDR2 as set forth in SEQ ID NO:82; and CDR3 as set forth in SEQ ID NO:83, wherein the nanobody comprises an unnatural amino acid in CDR1, CDR2, or CDR3. In embodiments, the nanobody comprises CDR1 as set forth in SEQ ID NO:81, CDR2 as set forth in SEQ ID NO:82; and CDR3 as set forth in SEQ ID NO:83, wherein the unnatural amino acid is at a position corresponding to position 5 or position 8 in SEQ ID NO:82; or the unnatural amino acid is at a position corresponding to 7 in SEQ ID NO:81. In embodiments, the unnatural amino acid is FSY, metaFSY, FFY, FSK, or meta-FSK. In embodiments, the nanobody comprises CDR1 as set forth in SEQ ID NO:81, CDR2 as set forth in SEQ ID NO:84 or SEQ ID NO:85; and CDR3 as set forth in SEQ ID NO: 83. In embodiments, the nanobody comprises CDR1 as set forth in SEQ ID NO:86, CDR2 as set forth in SEQ ID NO:82; and CDR3 as set forth in SEQ ID NO:83. In embodiments, the nanobody comprises CDR1 as set forth in SEQ ID NO:81, CDR2 as set forth in SEQ ID NO:87; and CDR3 as set forth in SEQ ID NO:83. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent. In embodiments, the disclosure provides nanobody NB13 as described herein, including embodiments thereof, covalently bonded to prostate-specific membrane antigen (PSMA). In embodiments, the disclosure provides nanobody NB13 as described herein, including embodiments thereof, covalently bonded to PSMA expressed on a cancer tumor.

Provided herein is nanobody NB17B05, wherein the nanobody comprises an unnatural amino acid. In embodiments, the nanobody comprises CDR1 as set forth in SEQ ID NO:93, CDR2 as set forth in SEQ ID NO:94; and CDR3 as set forth in SEQ ID NO:95, wherein the nanobody comprises an unnatural amino acid in CDR1, CDR2, or CDR3. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to any one of positions 8 to 16 in SEQ ID NO:94; or the unnatural amino acid is at a position corresponding to position 5 or 6 in SEQ ID NO:95. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 8 in SEQ ID NO:94. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 9 in SEQ ID NO:94. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 10 in SEQ ID NO:94. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 11 in SEQ ID NO: 94. In embodiments, the nanobody comprises CDR1 as set forth in SEQ ID NO:93, CDR2 as set forth in SEQ ID NO:94; and CDR3 as set forth in SEQ ID NO: 95, wherein the unnatural amino acid is at a position corresponding to position 12 in SEQ ID NO: 94. In embodiments, the nanobody comprises CDR1 as set forth in SEQ ID NO: 93, CDR2 as set forth in SEQ ID NO:94; and CDR3 as set forth in SEQ ID NO:95, wherein the unnatural amino acid is at a position corresponding to position 13 in SEQ ID NO: 94. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 14 in SEQ ID NO:94. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 15 in SEQ ID NO:94. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 16 in SEQ ID NO:94. In embodiments, the nanobody comprises CDR1 as set forth in SEQ ID NO: 93, CDR2 as set forth in SEQ ID NO:94; and CDR3 as set forth in SEQ ID NO:95, wherein the unnatural amino acid is at a position corresponding to position 5 in SEQ ID NO:95. In embodiments, the nanobody comprises CDR1 as set forth in SEQ ID NO:93, CDR2 as set forth in SEQ ID NO:94; and CDR3 as set forth in SEQ ID NO:95, wherein the unnatural amino acid is at a position corresponding to position 6 in SEQ ID NO:95. In embodiments, the unnatural amino acid is FSY, metaFSY, FFY, FSK, or meta-FSK. In embodiments, the nanobody comprises CDR1 as set forth in SEQ ID NO:93, CDR2 as set forth in any one of SEQ ID NOS:96-102 and 105-113; and CDR3 as set forth in SEQ ID NO:95. In embodiments, the nanobody comprises CDR1 as set forth in SEQ ID NO:93, CDR2 as set forth in any one of SEQ ID NO:94; and CDR3 as set forth in any one of SEQ ID NOS:103, 104, 114, or 115. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

Provided herein is nanobody A1, wherein the nanobody comprises an unnatural amino acid. In embodiments, the nanobody comprises CDR1 as set forth in SEQ ID NO:215, CDR2 as set forth in SEQ ID NO:216, and CDR3 as set forth in SEQ ID NO:217, wherein the nanobody comprises an unnatural amino acid in CDR1, CDR2, or CDR3. In embodiments, the unnatural amino acid is at a position corresponding to position 1, 3, 5, 6, or 8 in SEQ ID NO:215. In embodiments, the unnatural amino acid is at a position corresponding to position 4, 5, 6, or 8 in SEQ ID NO:217. In embodiments, the unnatural amino acid is FSY, metaFSY, FFY, FSK, or meta-FSK. In embodiments, the unnatural amino acid is metaFSY, FFY, or meta-FSK. In embodiments, the unnatural amino acid is metaFSY. In embodiments, the unnatural amino acid is FFY. In embodiments, the unnatural amino acid is meta-FSK. In embodiments, the unnatural amino acid is FSY, metaFSY, FFY, FSK, or meta-FSK. In embodiments, the nanobody comprises CDR1 as set forth in SEQ ID NO:218, 219, 220, 221, or 222, CDR2 as set forth in SEQ ID NO:216, or CDR3 as set forth in SEQ ID NO:217. In embodiments, the nanobody comprises CDR1 as set forth in SEQ ID NO:218, CDR2 as set forth in SEQ ID NO:216, or CDR3 as set forth in SEQ ID NO: 217. In embodiments, the nanobody comprises CDR1 as set forth in SEQ ID NO:219, CDR2 as set forth in SEQ ID NO:216, or CDR3 as set forth in SEQ ID NO:217. In embodiments, the nanobody comprises CDR1 as set forth in SEQ ID NO:220, CDR2 as set forth in SEQ ID NO:216, or CDR3 as set forth in SEQ ID NO:217. In embodiments, the nanobody comprises CDR1 as set forth in SEQ ID NO:221, CDR2 as set forth in SEQ ID NO:216, or CDR3 as set forth in SEQ ID NO:217. In embodiments, the nanobody comprises CDR1 as set forth in SEQ ID NO:222, CDR2 as set forth in SEQ ID NO:216, or CDR3 as set forth in SEQ ID NO:217. In embodiments, the nanobody comprises CDR1 as set forth in SEQ ID NO:215, CDR2 as set forth in SEQ ID NO:216, and CDR3 as set forth in SEQ ID NO:223, 224, 225, or 226. In embodiments, the nanobody comprises CDR1 as set forth in SEQ ID NO:215, CDR2 as set forth in SEQ ID NO:216, and CDR3 as set forth in SEQ ID NO:223. In embodiments, the nanobody comprises CDR1 as set forth in SEQ ID NO:215, CDR2 as set forth in SEQ ID NO:216, and CDR3 as set forth in SEQ ID NO:224. In embodiments, the nanobody comprises CDR1 as set forth in SEQ ID NO:215, CDR2 as set forth in SEQ ID NO:216, and CDR3 as set forth in SEQ ID NO:225. In embodiments, the nanobody comprises CDR1 as set forth in SEQ ID NO:215, CDR2 as set forth in SEQ ID NO:216, and CDR3 as set forth in SEQ ID NO:226. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent. In embodiments, the disclosure provides a biomolecule conjugate comprising nanobody A1 as described herein, including embodiments thereof, covalently bonded to mesothelin (MSLN). In embodiments, the biomolecule conjugate comprises nanobody A1 as described herein, including embodiments thereof, covalently bonded to MSLN expressed on a cancer tumor. In embodiments, the biomolecule conjugate comprises nanobody A1 as described herein, including embodiments thereof, covalently bonded to MSLN overexpressed on a cancer tumor.

Provided herein is nanobody C6, wherein the nanobody comprises an unnatural amino acid. In embodiments, the nanobody comprises CDR1 as set forth in SEQ ID NO:240, CDR2 as set forth in SEQ ID NO:241, and CDR3 as set forth in SEQ ID NO:242, wherein the nanobody comprises an unnatural amino acid in CDR1, CDR2, or CDR3. In embodiments, the unnatural amino acid is at a position corresponding to position 2, 4, 6, or 7 in SEQ ID NO:240. In embodiments, the unnatural amino acid is at a position corresponding to position 2, 3, 4, or 5 in SEQ ID NO:241. In embodiments, the unnatural amino acid is at a position corresponding to position 1, 6, 7, or 10 in SEQ ID NO:242. In embodiments, the unnatural amino acid is FSY, metaFSY, FFY, FSK, or meta-FSK. In embodiments, the unnatural amino acid is metaFSY, FFY, or meta-FSK. In embodiments, the unnatural amino acid is metaFSY. In embodiments, the unnatural amino acid is FFY. In embodiments, the unnatural amino acid is meta-FSK. In embodiments, the unnatural amino acid is FSY, metaFSY, FFY, FSK, or meta-FSK. In embodiments, the nanobody comprises CDR1 as set forth in SEQ ID NO:243, 244, 245, or 246, CDR2 as set forth in SEQ ID NO:241, and CDR3 as set forth in SEQ ID NO:242. In embodiments, the nanobody comprises CDR1 as set forth in SEQ ID NO:243, CDR2 as set forth in SEQ ID NO:241, and CDR3 as set forth in SEQ ID NO:242. In embodiments, the nanobody comprises CDR1 as set forth in SEQ ID NO:244, CDR2 as set forth in SEQ ID NO:241, and CDR3 as set forth in SEQ ID NO: 242. In embodiments, the nanobody comprises CDR1 as set forth in SEQ ID NO:245, CDR2 as set forth in SEQ ID NO:241, and CDR3 as set forth in SEQ ID NO:242. In embodiments, the nanobody comprises CDR1 as set forth in SEQ ID NO:246, CDR2 as set forth in SEQ ID NO:241, and CDR3 as set forth in SEQ ID NO:242. In embodiments, the nanobody comprises CDR1 as set forth in SEQ ID NO:240, CDR2 as set forth in SEQ ID NO:247, 248, 249, or 250, and CDR3 as set forth in SEQ ID NO:242. In embodiments, the nanobody comprises CDR1 as set forth in SEQ ID NO:240, CDR2 as set forth in SEQ ID NO:247, and CDR3 as set forth in SEQ ID NO:242. In embodiments, the nanobody comprises CDR1 as set forth in SEQ ID NO:240, CDR2 as set forth in SEQ ID NO:248, and CDR3 as set forth in SEQ ID NO:242. In embodiments, the nanobody comprises CDR1 as set forth in SEQ ID NO:240, CDR2 as set forth in SEQ ID NO:249, and CDR3 as set forth in SEQ ID NO:242. In embodiments, the nanobody comprises CDR1 as set forth in SEQ ID NO:240, CDR2 as set forth in SEQ ID NO:250, and CDR3 as set forth in SEQ ID NO:242. In embodiments, the nanobody comprises CDR1 as set forth in SEQ ID NO:240, CDR2 as set forth in SEQ ID NO:241, and CDR3 as set forth in SEQ ID NO:251, 252, 253, or 254. In embodiments, the nanobody comprises CDR1 as set forth in SEQ ID NO:240, CDR2 as set forth in SEQ ID NO:241, and CDR3 as set forth in SEQ ID NO:251. In embodiments, the nanobody comprises CDR1 as set forth in SEQ ID NO:240, CDR2 as set forth in SEQ ID NO:241, and CDR3 as set forth in SEQ ID NO:252. In embodiments, the nanobody comprises CDR1 as set forth in SEQ ID NO:240, CDR2 as set forth in SEQ ID NO:241, and CDR3 as set forth in SEQ ID NO:253. In embodiments, the nanobody comprises CDR1 as set forth in SEQ ID NO:240, CDR2 as set forth in SEQ ID NO:241, and CDR3 as set forth in SEQ ID NO:254. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent. In embodiments, the disclosure provides a biomolecule conjugate comprising nanobody C6 as described herein, including embodiments thereof, covalently bonded to MSLN. In embodiments, the biomolecule conjugate comprises nanobody C6 as described herein, including embodiments thereof, covalently bonded to MSLN expressed on a cancer tumor. In embodiments, the biomolecule conjugate comprises nanobody C6 as described herein, including embodiments thereof, covalently bonded to MSLN overexpressed on a cancer tumor.

Provided herein is nanobody 7D12, wherein the nanobody comprises an unnatural amino acid. In embodiments, the nanobody comprises CDR1 as set forth in SEQ ID NO:155, CDR2 as set forth in SEQ ID NO:156; and CDR3 as set forth in SEQ ID NO:157, wherein the nanobody comprises an unnatural amino acid in CDR1, CDR2, or CDR3. In embodiments, the unnatural amino acid is FSY, metaFSY, FFY, FSK, or meta-FSK. In embodiments, the unnatural amino acid is metaFSY, FFY, or meta-FSK. In embodiments, the unnatural amino acid is metaFSY. In embodiments, the unnatural amino acid is FFY. In embodiments, the unnatural amino acid is meta-FSK. In embodiments, the unnatural amino acid is FSY, metaFSY, FFY, FSK, or meta-FSK. In embodiments, the nanobody comprises CDR1 as set forth in SEQ ID NO:155, CDR2 as set forth in any one of SEQ ID NO:156; and CDR3 as set forth in SEQ ID NO:181 or 182. In embodiments, the nanobody comprises CDR1 as set forth in SEQ ID NO:155, CDR2 as set forth in any one of SEQ ID NO:156; and CDR3 as set forth in SEQ ID NO:181. In embodiments, the nanobody comprises CDR1 as set forth in SEQ ID NO:155, CDR2 as set forth in any one of SEQ ID NO:156; and CDR3 as set forth in SEQ ID NO:182. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

Provided herein is nanobody SR4, wherein the nanobody comprises an unnatural amino acid. In embodiments, the nanobody comprises CDR1 as set forth in SEQ ID NO:31, CDR2 as set forth in SEQ ID NO:32; and CDR3 as set forth in SEQ ID NO:33, wherein the nanobody comprises an unnatural amino acid. In embodiments, the nanobody comprises CDR1 as set forth in SEQ ID NO:31, CDR2 as set forth in SEQ ID NO:32; and CDR3 as set forth in SEQ ID NO:33, wherein the nanobody comprises an unnatural amino acid in CDR1, CDR2, or CDR3. In embodiments, the unnatural amino acid is FSY, metaFSY, FFY, FSK, or meta-FSK. In embodiments, the nanobody comprises CDR1 as set forth in SEQ ID NO:31, CDR2 as set forth in SEQ ID NO:32; and CDR3 as set forth in SEQ ID NO:33, wherein the unnatural amino acid is at a position corresponding to position 5 or position 8 in SEQ ID NO:32. In embodiments, the nanobody comprises CDR1 as set forth in SEQ ID NO:31, CDR2 as set forth in SEQ ID NO:268; and CDR3 as set forth in SEQ ID NO:33. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

Provided herein is nanobody MR17K99Y, wherein the nanobody comprises an unnatural amino acid. In embodiments, the nanobody comprises CDR1 as set forth in SEQ ID NO:35, CDR2 as set forth in SEQ ID NO:36; and CDR3 as set forth in SEQ ID NO:37, wherein the nanobody comprises an unnatural amino acid. In embodiments, the nanobody comprises CDR1 as set forth in SEQ ID NO:35, CDR2 as set forth in SEQ ID NO:36; and CDR3 as set forth in SEQ ID NO:37, wherein the nanobody comprises an unnatural amino acid in CDR1, CDR2, or CDR3. In embodiments, the nanobody comprises CDR1 as set forth in SEQ ID NO:35, CDR2 as set forth in SEQ ID NO:36; and CDR3 as set forth in SEQ ID NO:37, wherein the unnatural amino acid is at a position corresponding to position 4 in SEQ ID NO:37. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

Provided herein is nanobody H11D4, wherein the nanobody comprises an unnatural amino acid. In embodiments, the nanobody comprises CDR1 as set forth in SEQ ID NO:39, CDR2 as set forth in SEQ ID NO:40; and CDR3 as set forth in SEQ ID NO:41, wherein the nanobody comprises an unnatural amino acid. In embodiments, the nanobody comprises CDR1 as set forth in SEQ ID NO:39, CDR2 as set forth in SEQ ID NO:40; and CDR3 as set forth in SEQ ID NO:41, wherein the nanobody comprises an unnatural amino acid in CDR1, CDR2, or CDR3. In embodiments, the nanobody comprises CDR1 as set forth in SEQ ID NO:39, CDR2 as set forth in SEQ ID NO:40; and CDR3 as set forth in SEQ ID NO:41, wherein the unnatural amino acid is at a position corresponding to position 18 or position 19 in SEQ ID NO:41. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

Provided herein are nanobodies having an amino acid sequence with at least 90% sequence identity to any one of SEQ ID NOS:65, 73, 79, 88, 89, 90, 91, 116-127, 183-189, 227-238, and 255-266; provided that the nanobody has 100% sequence identity with CDR1, CDR2, and CDR3 therein. In embodiments, the nanobodies have an amino acid sequence with at least 95% sequence identity to any one of SEQ ID NOS:65, 73, 79, 88, 89, 90, 91, 116-127, 183-189, 227-238, and 255-266; provided that the nanobody has 100% sequence identity with CDR1, CDR2, and CDR3 therein. In embodiments, the nanobodies have an amino acid sequence as set forth in any one of SEQ ID NOS:65, 73, 79, 88, 89, 90, 91, 116-127, 183-189, 227-238, and 255-266. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:65. In embodiments, the nanobody is as set forth in SEQ ID NO:65. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:65. In embodiments, the nanobody comprises an amino acid sequence with at least 85% sequence identity to SEQ ID NO:65. In embodiments, the nanobody comprises an amino acid sequence with at least 90% sequence identity to SEQ ID NO:65. In embodiments, the nanobody comprises an amino acid sequence with at least 92% sequence identity to SEQ ID NO:65. In embodiments, the nanobody comprises an amino acid sequence with at least 94% sequence identity to SEQ ID NO:65. In embodiments, the nanobody comprises an amino acid sequence with at least 95% sequence identity to SEQ ID NO:65. In embodiments, the nanobody comprises an amino acid sequence with at least 96% sequence identity to SEQ ID NO:65. In embodiments, the nanobody comprises an amino acid sequence with at least 98% sequence identity to SEQ ID NO:65. In embodiments, when the nanobody comprises an amino acid sequence having less than 100% sequence identity to SEQ ID NO:65, then SEQ ID NO:65 has 100% sequence identity with the CDRs therein. In embodiments, SEQ ID NO:65 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent. In embodiments, the disclosure provides a biomolecule conjugate comprising SEQ ID NO:65 covalently bonded to a coronavirus. In embodiments, the disclosure provides a biomolecule conjugate comprising SEQ ID NO:65 covalently bonded to SARS-CoV. In embodiments, the disclosure provides a biomolecule conjugate comprising SEQ ID NO:65 covalently bonded to SARS-CoV-2. In embodiments, the disclosure provides a method of treating COVID-19 in a patient in need thereof comprising administering to a patient an effective amount of SEQ ID NO:65 as described herein, including embodiments thereof. In embodiments, the disclosure provides a method of treating a coronavirus infection in a patient in need thereof comprising administering to a patient an effective amount of SEQ ID NO:65 as described herein, including embodiments thereof. In embodiments, the disclosure provides a method of treating a SARS-CoV-2 infection in a patient in need thereof comprising administering to a patient an effective amount of SEQ ID NO:65 as described herein, including embodiments thereof.

In embodiments, the nanobody comprises the amino acid sequence of SEQ ID NO:72. In embodiments, the nanobody is as set forth in SEQ ID NO:72. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:72. In embodiments, the nanobody comprises an amino acid sequence with at least 85% sequence identity to SEQ ID NO:72. In embodiments, the nanobody comprises an amino acid sequence with at least 90% sequence identity to SEQ ID NO:72. In embodiments, the nanobody comprises an amino acid sequence with at least 92% sequence identity to SEQ ID NO:72. In embodiments, the nanobody comprises an amino acid sequence with at least 94% sequence identity to SEQ ID NO:72. In embodiments, the nanobody comprises an amino acid sequence with at least 95% sequence identity to SEQ ID NO:72. In embodiments, the nanobody comprises an amino acid sequence with at least 96% sequence identity to SEQ ID NO:72. In embodiments, the nanobody comprises an amino acid sequence with at least 98% sequence identity to SEQ ID NO:72. In embodiments, when the nanobody comprises an amino acid sequence having less than 100% sequence identity to SEQ ID NO:72, then SEQ ID NO:72 has 100% sequence identity with the CDRs therein. In embodiments, SEQ ID NO:72 further comprises a His6-tag at the C-terminus. In embodiments, the nanobody further comprises a detectable agent. In embodiments, the nanobody further comprises a radioisotope. In embodiments, the radioisotope is a positron-emitting radioisotope. In embodiments, the positron-emitting radioisotope is ¹¹C, ¹³N, 150 ¹⁸F, ⁶⁴Cu, ⁶⁸Ga, ⁷⁸Br, ⁸²Rb, ⁸⁶Y, ⁸⁹Zr, ⁹⁰Y, ²²Na, ²⁶Al, ⁴⁰K, ⁸³Sr, or ¹²⁴I. In embodiments, the positron-emitting radioisotope is ¹²⁴I. In embodiments, the radioisotope is an alpha-emitting radioisotope. In embodiments, the alpha-emitting radioisotope is ²¹¹At, ²²⁷Th, ²²⁵Ac, ²²³Ra, ²¹³Bi, or ²¹²Bi. In embodiments, the alpha-emitting radioisotope is ²¹¹At. In embodiments, the nanobody further comprises a therapeutic agent. In embodiments, the nanobody further comprises a detectable agent and a therapeutic agent. In embodiments, the disclosure provides a biomolecule conjugate comprising SEQ ID NO:72 as described herein, including embodiments thereof, covalently bonded to HER2. In embodiments, the disclosure provides a biomolecule conjugate comprising SEQ ID NO:72 as described herein, including embodiments thereof, covalently bonded to HER2 expressed on a cancer tumor.

In embodiments, the nanobody comprises the amino acid sequence of SEQ ID NO:73. In embodiments, the nanobody is as set forth in SEQ ID NO:73. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:73. In embodiments, the nanobody comprises an amino acid sequence with at least 85% sequence identity to SEQ ID NO:73. In embodiments, the nanobody comprises an amino acid sequence with at least 90% sequence identity to SEQ ID NO:73. In embodiments, the nanobody comprises an amino acid sequence with at least 92% sequence identity to SEQ ID NO:73. In embodiments, the nanobody comprises an amino acid sequence with at least 94% sequence identity to SEQ ID NO:73. In embodiments, the nanobody comprises an amino acid sequence with at least 95% sequence identity to SEQ ID NO:73. In embodiments, the nanobody comprises an amino acid sequence with at least 96% sequence identity to SEQ ID NO:73. In embodiments, the nanobody comprises an amino acid sequence with at least 98% sequence identity to SEQ ID NO:73. In embodiments, when the nanobody comprises an amino acid sequence having less than 100% sequence identity to SEQ ID NO:73, then SEQ ID NO:73 has 100% sequence identity with the CDRs therein. In embodiments, SEQ ID NO:73 further comprises a His6-tag at the C-terminus. In embodiments, the nanobody further comprises a detectable agent. In embodiments, the nanobody further comprises a radioisotope. In embodiments, the radioisotope is a positron-emitting radioisotope. In embodiments, the positron-emitting radioisotope is ¹¹C, ¹³N, ¹⁵O, ¹⁸F, ⁶⁴Cu, ⁶⁸Ga, ⁷⁸Br, ⁸²Rb, ⁸⁶Y, ⁸⁹Zr, ⁹⁰Y, ²²Na, ²⁶Al, ⁴⁰K, ⁸³Sr, or ¹²⁴I. In embodiments, the positron-emitting radioisotope is ¹²⁴I. In embodiments, the radioisotope is an alpha-emitting radioisotope. In embodiments, the alpha-emitting radioisotope is ²¹¹At, ²²⁷Th, ²²⁵Ac, ²²³Ra, ²¹³Bi, or ²¹²Bi. In embodiments, the alpha-emitting radioisotope is ²¹¹At. In embodiments, the nanobody further comprises a therapeutic agent. In embodiments, the nanobody further comprises a detectable agent and a therapeutic agent. In embodiments, the disclosure provides a biomolecule conjugate comprising SEQ ID NO:73 as described herein, including embodiments thereof, covalently bonded to HER2. In embodiments, the disclosure provides a biomolecule conjugate comprising SEQ ID NO:73 as described herein, including embodiments thereof, covalently bonded to HER2 expressed on a cancer tumor.

In embodiments, the nanobody comprises the amino acid sequence of SEQ ID NO:79. In embodiments, the nanobody is as set forth in SEQ ID NO:79. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:79. In embodiments, the nanobody comprises an amino acid sequence with at least 85% sequence identity to SEQ ID NO:79. In embodiments, the nanobody comprises an amino acid sequence with at least 90% sequence identity to SEQ ID NO:79. In embodiments, the nanobody comprises an amino acid sequence with at least 92% sequence identity to SEQ ID NO:79. In embodiments, the nanobody comprises an amino acid sequence with at least 94% sequence identity to SEQ ID NO:79. In embodiments, the nanobody comprises an amino acid sequence with at least 95% sequence identity to SEQ ID NO:79. In embodiments, the nanobody comprises an amino acid sequence with at least 96% sequence identity to SEQ ID NO:79. In embodiments, the nanobody comprises an amino acid sequence with at least 98% sequence identity to SEQ ID NO:79. In embodiments, when the nanobody comprises an amino acid sequence having less than 100% sequence identity to SEQ ID NO:79, then SEQ ID NO:79 has 100% sequence identity with the CDRs therein. In embodiments, SEQ ID NO:79 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:88. In embodiments, the nanobody is as set forth in SEQ ID NO:88. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:88. In embodiments, the nanobody comprises an amino acid sequence with at least 85% sequence identity to SEQ ID NO:88. In embodiments, the nanobody comprises an amino acid sequence with at least 90% sequence identity to SEQ ID NO:88. In embodiments, the nanobody comprises an amino acid sequence with at least 92% sequence identity to SEQ ID NO:88. In embodiments, the nanobody comprises an amino acid sequence with at least 94% sequence identity to SEQ ID NO:88. In embodiments, the nanobody comprises an amino acid sequence with at least 95% sequence identity to SEQ ID NO:88. In embodiments, the nanobody comprises an amino acid sequence with at least 96% sequence identity to SEQ ID NO:88. In embodiments, the nanobody comprises an amino acid sequence with at least 98% sequence identity to SEQ ID NO:88. In embodiments, when the nanobody comprises an amino acid sequence having less than 100% sequence identity to SEQ ID NO:88, then SEQ ID NO:88 has 100% sequence identity with the CDRs therein. In embodiments, SEQ ID NO:88 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:89. In embodiments, the nanobody is as set forth in SEQ ID NO:89. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:89. In embodiments, the nanobody comprises an amino acid sequence with at least 85% sequence identity to SEQ ID NO:89. In embodiments, the nanobody comprises an amino acid sequence with at least 90% sequence identity to SEQ ID NO:89. In embodiments, the nanobody comprises an amino acid sequence with at least 92% sequence identity to SEQ ID NO:89. In embodiments, the nanobody comprises an amino acid sequence with at least 94% sequence identity to SEQ ID NO:89. In embodiments, the nanobody comprises an amino acid sequence with at least 95% sequence identity to SEQ ID NO:89. In embodiments, the nanobody comprises an amino acid sequence with at least 96% sequence identity to SEQ ID NO:89. In embodiments, the nanobody comprises an amino acid sequence with at least 98% sequence identity to SEQ ID NO:89. In embodiments, when the nanobody comprises an amino acid sequence having less than 100% sequence identity to SEQ ID NO:89, then SEQ ID NO:89 has 100% sequence identity with the CDRs therein. In embodiments, SEQ ID NO:89 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:90. In embodiments, the nanobody is as set forth in SEQ ID NO:90. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:90. In embodiments, the nanobody comprises an amino acid sequence with at least 85% sequence identity to SEQ ID NO:90. In embodiments, the nanobody comprises an amino acid sequence with at least 90% sequence identity to SEQ ID NO:90. In embodiments, the nanobody comprises an amino acid sequence with at least 92% sequence identity to SEQ ID NO:90. In embodiments, the nanobody comprises an amino acid sequence with at least 94% sequence identity to SEQ ID NO:90. In embodiments, the nanobody comprises an amino acid sequence with at least 95% sequence identity to SEQ ID NO:90. In embodiments, the nanobody comprises an amino acid sequence with at least 96% sequence identity to SEQ ID NO:90. In embodiments, the nanobody comprises an amino acid sequence with at least 98% sequence identity to SEQ ID NO:90. In embodiments, when the nanobody comprises an amino acid sequence having less than 100% sequence identity to SEQ ID NO:90, then SEQ ID NO:90 has 100% sequence identity with the CDRs therein. In embodiments, SEQ ID NO:90 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:91. In embodiments, the nanobody is as set forth in SEQ ID NO:91. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:91. In embodiments, the nanobody comprises an amino acid sequence with at least 85% sequence identity to SEQ ID NO:91. In embodiments, the nanobody comprises an amino acid sequence with at least 91% sequence identity to SEQ ID NO:91. In embodiments, the nanobody comprises an amino acid sequence with at least 92% sequence identity to SEQ ID NO:91. In embodiments, the nanobody comprises an amino acid sequence with at least 94% sequence identity to SEQ ID NO:91. In embodiments, the nanobody comprises an amino acid sequence with at least 95% sequence identity to SEQ ID NO:91. In embodiments, the nanobody comprises an amino acid sequence with at least 96% sequence identity to SEQ ID NO:91. In embodiments, the nanobody comprises an amino acid sequence with at least 98% sequence identity to SEQ ID NO:91. In embodiments, when the nanobody comprises an amino acid sequence having less than 100% sequence identity to SEQ ID NO:91, then SEQ ID NO:91 has 100% sequence identity with the CDRs therein. In embodiments, SEQ ID NO:91 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the disclosure provides any one of SEQ ID NOS:88-91 as described herein, including embodiments thereof, covalently bonded to prostate-specific membrane antigen (PSMA). In embodiments, the disclosure provides any one of SEQ ID NOS:88-91 as described herein, including embodiments thereof, covalently bonded to PSMA expressed on a cancer tumor.

In embodiments, the nanobody comprises the amino acid sequence of SEQ ID NO:116. In embodiments, the nanobody is as set forth in SEQ ID NO:116. In embodiments, the nanobody comprises an amino acid sequence with at least 85%, 86%, 87%, 88%, 89%, 90%, 116%, 92%, 93%, 94%, 95%, 96%, 97%, 98%, 99%, or 100% sequence identity to SEQ ID NO:116. In embodiments, the nanobody comprises an amino acid sequence with at least 85% sequence identity to SEQ ID NO:116. In embodiments, the nanobody comprises an amino acid sequence with at least 116% sequence identity to SEQ ID NO:116. In embodiments, the nanobody comprises an amino acid sequence with at least 92% sequence identity to SEQ ID NO:116. In embodiments, the nanobody comprises an amino acid sequence with at least 94% sequence identity to SEQ ID NO:116. In embodiments, the nanobody comprises an amino acid sequence with at least 95% sequence identity to SEQ ID NO:116. In embodiments, the nanobody comprises an amino acid sequence with at least 96% sequence identity to SEQ ID NO:116. In embodiments, the nanobody comprises an amino acid sequence with at least 98% sequence identity to SEQ ID NO:116. In embodiments, when the nanobody comprises an amino acid sequence having less than 100% sequence identity to SEQ ID NO:116, then SEQ ID NO:116 has 100% sequence identity with the CDRs therein. In embodiments, SEQ ID NO: 116 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:117. In embodiments, the nanobody is as set forth in SEQ ID NO:117. In embodiments, the nanobody comprises an amino acid sequence with at least 85%, 86%, 87%, 88%, 89%, 90%, 117%, 92%, 93%, 94%, 95%, 96%, 97%, 98%, 99%, or 100% sequence identity to SEQ ID NO:117. In embodiments, the nanobody comprises an amino acid sequence with at least 85% sequence identity to SEQ ID NO:117. In embodiments, the nanobody comprises an amino acid sequence with at least 117% sequence identity to SEQ ID NO:117. In embodiments, the nanobody comprises an amino acid sequence with at least 92% sequence identity to SEQ ID NO:117. In embodiments, the nanobody comprises an amino acid sequence with at least 94% sequence identity to SEQ ID NO:117. In embodiments, the nanobody comprises an amino acid sequence with at least 95% sequence identity to SEQ ID NO:117. In embodiments, the nanobody comprises an amino acid sequence with at least 96% sequence identity to SEQ ID NO:117. In embodiments, the nanobody comprises an amino acid sequence with at least 98% sequence identity to SEQ ID NO:117. In embodiments, when the nanobody comprises an amino acid sequence having less than 100% sequence identity to SEQ ID NO:117, then SEQ ID NO:117 has 100% sequence identity with the CDRs therein. In embodiments, SEQ ID NO: 117 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:118. In embodiments, the nanobody is as set forth in SEQ ID NO:118. In embodiments, the nanobody comprises an amino acid sequence with at least 85%, 86%, 87%, 88%, 89%, 90%, 118%, 92%, 93%, 94%, 95%, 96%, 97%, 98%, 99%, or 100% sequence identity to SEQ ID NO:118. In embodiments, the nanobody comprises an amino acid sequence with at least 85% sequence identity to SEQ ID NO:118. In embodiments, the nanobody comprises an amino acid sequence with at least 118% sequence identity to SEQ ID NO:118. In embodiments, the nanobody comprises an amino acid sequence with at least 92% sequence identity to SEQ ID NO:118. In embodiments, the nanobody comprises an amino acid sequence with at least 94% sequence identity to SEQ ID NO:118. In embodiments, the nanobody comprises an amino acid sequence with at least 95% sequence identity to SEQ ID NO:118. In embodiments, the nanobody comprises an amino acid sequence with at least 96% sequence identity to SEQ ID NO:118. In embodiments, the nanobody comprises an amino acid sequence with at least 98% sequence identity to SEQ ID NO:118. In embodiments, when the nanobody comprises an amino acid sequence having less than 100% sequence identity to SEQ ID NO:118, then SEQ ID NO:118 has 100% sequence identity with the CDRs therein. In embodiments, SEQ ID NO:118 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:119. In embodiments, the nanobody is as set forth in SEQ ID NO:119. In embodiments, the nanobody comprises an amino acid sequence with at least 85%, 86%, 87%, 88%, 89%, 90%, 119%, 92%, 93%, 94%, 95%, 96%, 97%, 98%, 99%, or 100% sequence identity to SEQ ID NO:119. In embodiments, the nanobody comprises an amino acid sequence with at least 85% sequence identity to SEQ ID NO:119. In embodiments, the nanobody comprises an amino acid sequence with at least 119% sequence identity to SEQ ID NO:119. In embodiments, the nanobody comprises an amino acid sequence with at least 92% sequence identity to SEQ ID NO:119. In embodiments, the nanobody comprises an amino acid sequence with at least 94% sequence identity to SEQ ID NO:119. In embodiments, the nanobody comprises an amino acid sequence with at least 95% sequence identity to SEQ ID NO:119. In embodiments, the nanobody comprises an amino acid sequence with at least 96% sequence identity to SEQ ID NO:119. In embodiments, the nanobody comprises an amino acid sequence with at least 98% sequence identity to SEQ ID NO:119. In embodiments, when the nanobody comprises an amino acid sequence having less than 100% sequence identity to SEQ ID NO:119, then SEQ ID NO:119 has 100% sequence identity with the CDRs therein. In embodiments, SEQ ID NO: 119 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:120. In embodiments, the nanobody is as set forth in SEQ ID NO:120. In embodiments, the nanobody comprises an amino acid sequence with at least 85%, 86%, 87%, 88%, 89%, 90%, 120%, 92%, 93%, 94%, 95%, 96%, 97%, 98%, 99%, or 100% sequence identity to SEQ ID NO:120. In embodiments, the nanobody comprises an amino acid sequence with at least 85% sequence identity to SEQ ID NO:120. In embodiments, the nanobody comprises an amino acid sequence with at least 120% sequence identity to SEQ ID NO:120. In embodiments, the nanobody comprises an amino acid sequence with at least 92% sequence identity to SEQ ID NO:120. In embodiments, the nanobody comprises an amino acid sequence with at least 94% sequence identity to SEQ ID NO:120. In embodiments, the nanobody comprises an amino acid sequence with at least 95% sequence identity to SEQ ID NO:120. In embodiments, the nanobody comprises an amino acid sequence with at least 96% sequence identity to SEQ ID NO:120. In embodiments, the nanobody comprises an amino acid sequence with at least 98% sequence identity to SEQ ID NO:120. In embodiments, when the nanobody comprises an amino acid sequence having less than 100% sequence identity to SEQ ID NO:120, then SEQ ID NO:120 has 100% sequence identity with the CDRs therein. In embodiments, SEQ ID NO: 120 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:121. In embodiments, the nanobody is as set forth in SEQ ID NO:121. In embodiments, the nanobody comprises an amino acid sequence with at least 85%, 86%, 87%, 88%, 89%, 90%, 121%, 92%, 93%, 94%, 95%, 96%, 97%, 98%, 99%, or 100% sequence identity to SEQ ID NO:121. In embodiments, the nanobody comprises an amino acid sequence with at least 85% sequence identity to SEQ ID NO:121. In embodiments, the nanobody comprises an amino acid sequence with at least 121% sequence identity to SEQ ID NO:121. In embodiments, the nanobody comprises an amino acid sequence with at least 92% sequence identity to SEQ ID NO:121. In embodiments, the nanobody comprises an amino acid sequence with at least 94% sequence identity to SEQ ID NO:121. In embodiments, the nanobody comprises an amino acid sequence with at least 95% sequence identity to SEQ ID NO:121. In embodiments, the nanobody comprises an amino acid sequence with at least 96% sequence identity to SEQ ID NO:121. In embodiments, the nanobody comprises an amino acid sequence with at least 98% sequence identity to SEQ ID NO:121. In embodiments, when the nanobody comprises an amino acid sequence having less than 100% sequence identity to SEQ ID NO:121, then SEQ ID NO:121 has 100% sequence identity with the CDRs therein. In embodiments, SEQ ID NO: 121 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:122. In embodiments, the nanobody is as set forth in SEQ ID NO:122. In embodiments, the nanobody comprises an amino acid sequence with at least 85%, 86%, 87%, 88%, 89%, 90%, 122%, 92%, 93%, 94%, 95%, 96%, 97%, 98%, 99%, or 100% sequence identity to SEQ ID NO:122. In embodiments, the nanobody comprises an amino acid sequence with at least 85% sequence identity to SEQ ID NO:122. In embodiments, the nanobody comprises an amino acid sequence with at least 122% sequence identity to SEQ ID NO:122. In embodiments, the nanobody comprises an amino acid sequence with at least 92% sequence identity to SEQ ID NO:122. In embodiments, the nanobody comprises an amino acid sequence with at least 94% sequence identity to SEQ ID NO:122. In embodiments, the nanobody comprises an amino acid sequence with at least 95% sequence identity to SEQ ID NO:122. In embodiments, the nanobody comprises an amino acid sequence with at least 96% sequence identity to SEQ ID NO:122. In embodiments, the nanobody comprises an amino acid sequence with at least 98% sequence identity to SEQ ID NO:122. In embodiments, when the nanobody comprises an amino acid sequence having less than 100% sequence identity to SEQ ID NO:122, then SEQ ID NO:122 has 100% sequence identity with the CDRs therein. In embodiments, SEQ ID NO: 122 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:123. In embodiments, the nanobody is as set forth in SEQ ID NO:123. In embodiments, the nanobody comprises an amino acid sequence with at least 85%, 86%, 87%, 88%, 89%, 90%, 123%, 92%, 93%, 94%, 95%, 96%, 97%, 98%, 99%, or 100% sequence identity to SEQ ID NO:123. In embodiments, the nanobody comprises an amino acid sequence with at least 85% sequence identity to SEQ ID NO:123. In embodiments, the nanobody comprises an amino acid sequence with at least 123% sequence identity to SEQ ID NO:123. In embodiments, the nanobody comprises an amino acid sequence with at least 92% sequence identity to SEQ ID NO:123. In embodiments, the nanobody comprises an amino acid sequence with at least 94% sequence identity to SEQ ID NO:123. In embodiments, the nanobody comprises an amino acid sequence with at least 95% sequence identity to SEQ ID NO:123. In embodiments, the nanobody comprises an amino acid sequence with at least 96% sequence identity to SEQ ID NO:123. In embodiments, the nanobody comprises an amino acid sequence with at least 98% sequence identity to SEQ ID NO:123. In embodiments, when the nanobody comprises an amino acid sequence having less than 100% sequence identity to SEQ ID NO:123, then SEQ ID NO:123 has 100% sequence identity with the CDRs therein. In embodiments, SEQ ID NO: 123 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:124. In embodiments, the nanobody is as set forth in SEQ ID NO:124. In embodiments, the nanobody comprises an amino acid sequence with at least 85%, 86%, 87%, 88%, 89%, 90%, 124%, 92%, 93%, 94%, 95%, 96%, 97%, 98%, 99%, or 100% sequence identity to SEQ ID NO:124. In embodiments, the nanobody comprises an amino acid sequence with at least 85% sequence identity to SEQ ID NO:124. In embodiments, the nanobody comprises an amino acid sequence with at least 124% sequence identity to SEQ ID NO:124. In embodiments, the nanobody comprises an amino acid sequence with at least 92% sequence identity to SEQ ID NO:124. In embodiments, the nanobody comprises an amino acid sequence with at least 94% sequence identity to SEQ ID NO:124. In embodiments, the nanobody comprises an amino acid sequence with at least 95% sequence identity to SEQ ID NO:124. In embodiments, the nanobody comprises an amino acid sequence with at least 96% sequence identity to SEQ ID NO:124. In embodiments, the nanobody comprises an amino acid sequence with at least 98% sequence identity to SEQ ID NO:124. In embodiments, when the nanobody comprises an amino acid sequence having less than 100% sequence identity to SEQ ID NO:124, then SEQ ID NO:124 has 100% sequence identity with the CDRs therein. In embodiments, SEQ ID NO: 124 further comprises a His6-tag at the C-terminus. I In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:125. In embodiments, the nanobody is as set forth in SEQ ID NO:125. In embodiments, the nanobody comprises an amino acid sequence with at least 85%, 86%, 87%, 88%, 89%, 90%, 125%, 92%, 93%, 94%, 95%, 96%, 97%, 98%, 99%, or 100% sequence identity to SEQ ID NO:125. In embodiments, the nanobody comprises an amino acid sequence with at least 85% sequence identity to SEQ ID NO:125. In embodiments, the nanobody comprises an amino acid sequence with at least 125% sequence identity to SEQ ID NO:125. In embodiments, the nanobody comprises an amino acid sequence with at least 92% sequence identity to SEQ ID NO:125. In embodiments, the nanobody comprises an amino acid sequence with at least 94% sequence identity to SEQ ID NO:125. In embodiments, the nanobody comprises an amino acid sequence with at least 95% sequence identity to SEQ ID NO:125. In embodiments, the nanobody comprises an amino acid sequence with at least 96% sequence identity to SEQ ID NO:125. In embodiments, the nanobody comprises an amino acid sequence with at least 98% sequence identity to SEQ ID NO:125. In embodiments, when the nanobody comprises an amino acid sequence having less than 100% sequence identity to SEQ ID NO:125, then SEQ ID NO:125 has 100% sequence identity with the CDRs therein. In embodiments, SEQ ID NO: 125 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:126. In embodiments, the nanobody is as set forth in SEQ ID NO:126. In embodiments, the nanobody comprises an amino acid sequence with at least 85%, 86%, 87%, 88%, 89%, 90%, 126%, 92%, 93%, 94%, 95%, 96%, 97%, 98%, 99%, or 100% sequence identity to SEQ ID NO:126. In embodiments, the nanobody comprises an amino acid sequence with at least 85% sequence identity to SEQ ID NO:126. In embodiments, the nanobody comprises an amino acid sequence with at least 126% sequence identity to SEQ ID NO:126. In embodiments, the nanobody comprises an amino acid sequence with at least 92% sequence identity to SEQ ID NO:126. In embodiments, the nanobody comprises an amino acid sequence with at least 94% sequence identity to SEQ ID NO:126. In embodiments, the nanobody comprises an amino acid sequence with at least 95% sequence identity to SEQ ID NO:126. In embodiments, the nanobody comprises an amino acid sequence with at least 96% sequence identity to SEQ ID NO:126. In embodiments, the nanobody comprises an amino acid sequence with at least 98% sequence identity to SEQ ID NO:126. In embodiments, when the nanobody comprises an amino acid sequence having less than 100% sequence identity to SEQ ID NO:126, then SEQ ID NO:126 has 100% sequence identity with the CDRs therein. In embodiments, SEQ ID NO: 126 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:127. In embodiments, the nanobody is as set forth in SEQ ID NO:127. In embodiments, the nanobody comprises an amino acid sequence with at least 85%, 86%, 87%, 88%, 89%, 90%, 127%, 92%, 93%, 94%, 95%, 96%, 97%, 98%, 99%, or 100% sequence identity to SEQ ID NO:127. In embodiments, the nanobody comprises an amino acid sequence with at least 85% sequence identity to SEQ ID NO:127. In embodiments, the nanobody comprises an amino acid sequence with at least 127% sequence identity to SEQ ID NO:127. In embodiments, the nanobody comprises an amino acid sequence with at least 92% sequence identity to SEQ ID NO:127. In embodiments, the nanobody comprises an amino acid sequence with at least 94% sequence identity to SEQ ID NO:127. In embodiments, the nanobody comprises an amino acid sequence with at least 95% sequence identity to SEQ ID NO:127. In embodiments, the nanobody comprises an amino acid sequence with at least 96% sequence identity to SEQ ID NO:127. In embodiments, the nanobody comprises an amino acid sequence with at least 98% sequence identity to SEQ ID NO:127. In embodiments, when the nanobody comprises an amino acid sequence having less than 100% sequence identity to SEQ ID NO:127, then SEQ ID NO:127 has 100% sequence identity with the CDRs therein. In embodiments, SEQ ID NO: 127 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:183. In embodiments, the nanobody is as set forth in SEQ ID NO:183. In embodiments, the nanobody comprises an amino acid sequence with at least 85%, 86%, 87%, 88%, 89%, 90%, 183%, 92%, 93%, 94%, 95%, 96%, 97%, 98%, 99%, or 100% sequence identity to SEQ ID NO:183. In embodiments, the nanobody comprises an amino acid sequence with at least 85% sequence identity to SEQ ID NO:183. In embodiments, the nanobody comprises an amino acid sequence with at least 183% sequence identity to SEQ ID NO:183. In embodiments, the nanobody comprises an amino acid sequence with at least 92% sequence identity to SEQ ID NO:183. In embodiments, the nanobody comprises an amino acid sequence with at least 94% sequence identity to SEQ ID NO:183. In embodiments, the nanobody comprises an amino acid sequence with at least 95% sequence identity to SEQ ID NO:183. In embodiments, the nanobody comprises an amino acid sequence with at least 96% sequence identity to SEQ ID NO:183. In embodiments, the nanobody comprises an amino acid sequence with at least 98% sequence identity to SEQ ID NO:183. In embodiments, when the nanobody comprises an amino acid sequence having less than 100% sequence identity to SEQ ID NO:183, then SEQ ID NO:183 has 100% sequence identity with the CDRs therein. In embodiments, SEQ ID NO: 183 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:184. In embodiments, the nanobody is as set forth in SEQ ID NO:184. In embodiments, the nanobody comprises an amino acid sequence with at least 85%, 86%, 87%, 88%, 89%, 90%, 184%, 92%, 93%, 94%, 95%, 96%, 97%, 98%, 99%, or 100% sequence identity to SEQ ID NO:184. In embodiments, the nanobody comprises an amino acid sequence with at least 85% sequence identity to SEQ ID NO:184. In embodiments, the nanobody comprises an amino acid sequence with at least 184% sequence identity to SEQ ID NO:184. In embodiments, the nanobody comprises an amino acid sequence with at least 92% sequence identity to SEQ ID NO:184. In embodiments, the nanobody comprises an amino acid sequence with at least 94% sequence identity to SEQ ID NO:184. In embodiments, the nanobody comprises an amino acid sequence with at least 95% sequence identity to SEQ ID NO:184. In embodiments, the nanobody comprises an amino acid sequence with at least 96% sequence identity to SEQ ID NO:184. In embodiments, the nanobody comprises an amino acid sequence with at least 98% sequence identity to SEQ ID NO:184. In embodiments, when the nanobody comprises an amino acid sequence having less than 100% sequence identity to SEQ ID NO:184, then SEQ ID NO:184 has 100% sequence identity with the CDRs therein. In embodiments, SEQ ID NO: 184 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:185. In embodiments, the nanobody is as set forth in SEQ ID NO:185. In embodiments, the nanobody comprises an amino acid sequence with at least 85%, 86%, 87%, 88%, 89%, 90%, 185%, 92%, 93%, 94%, 95%, 96%, 97%, 98%, 99%, or 100% sequence identity to SEQ ID NO:185. In embodiments, the nanobody comprises an amino acid sequence with at least 8⁵% sequence identity to SEQ ID NO:185. In embodiments, the nanobody comprises an amino acid sequence with at least 185% sequence identity to SEQ ID NO:185. In embodiments, the nanobody comprises an amino acid sequence with at least 92% sequence identity to SEQ ID NO:185. In embodiments, the nanobody comprises an amino acid sequence with at least 94% sequence identity to SEQ ID NO:185. In embodiments, the nanobody comprises an amino acid sequence with at least 95% sequence identity to SEQ ID NO:185. In embodiments, the nanobody comprises an amino acid sequence with at least 96% sequence identity to SEQ ID NO:185. In embodiments, the nanobody comprises an amino acid sequence with at least 98% sequence identity to SEQ ID NO:185. In embodiments, when the nanobody comprises an amino acid sequence having less than 100% sequence identity to SEQ ID NO:185, then SEQ ID NO:185 has 100% sequence identity with the CDRs therein. In embodiments, SEQ ID NO: 185 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:186. In embodiments, the nanobody is as set forth in SEQ ID NO:186. In embodiments, the nanobody comprises an amino acid sequence with at least 85%, 86%, 87%, 88%, 89%, 90%, 186%, 92%, 93%, 94%, 95%, 96%, 97%, 98%, 99%, or 100% sequence identity to SEQ ID NO:186. In embodiments, the nanobody comprises an amino acid sequence with at least 85% sequence identity to SEQ ID NO:186. In embodiments, the nanobody comprises an amino acid sequence with at least 186% sequence identity to SEQ ID NO:186. In embodiments, the nanobody comprises an amino acid sequence with at least 92% sequence identity to SEQ ID NO:186. In embodiments, the nanobody comprises an amino acid sequence with at least 94% sequence identity to SEQ ID NO:186. In embodiments, the nanobody comprises an amino acid sequence with at least 95% sequence identity to SEQ ID NO:186. In embodiments, the nanobody comprises an amino acid sequence with at least 96% sequence identity to SEQ ID NO:186. In embodiments, the nanobody comprises an amino acid sequence with at least 98% sequence identity to SEQ ID NO:186. In embodiments, when the nanobody comprises an amino acid sequence having less than 100% sequence identity to SEQ ID NO:186, then SEQ ID NO:186 has 100% sequence identity with the CDRs therein. In embodiments, SEQ ID NO: 186 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:187. In embodiments, the nanobody is as set forth in SEQ ID NO:187. In embodiments, the nanobody comprises an amino acid sequence with at least 85%, 86%, 87%, 88%, 89%, 90%, 187%, 92%, 93%, 94%, 95%, 96%, 97%, 98%, 99%, or 100% sequence identity to SEQ ID NO:187. In embodiments, the nanobody comprises an amino acid sequence with at least 85% sequence identity to SEQ ID NO:187. In embodiments, the nanobody comprises an amino acid sequence with at least 187% sequence identity to SEQ ID NO:187. In embodiments, the nanobody comprises an amino acid sequence with at least 92% sequence identity to SEQ ID NO:187. In embodiments, the nanobody comprises an amino acid sequence with at least 94% sequence identity to SEQ ID NO:187. In embodiments, the nanobody comprises an amino acid sequence with at least 95% sequence identity to SEQ ID NO:187. In embodiments, the nanobody comprises an amino acid sequence with at least 96% sequence identity to SEQ ID NO:187. In embodiments, the nanobody comprises an amino acid sequence with at least 98% sequence identity to SEQ ID NO:187. In embodiments, when the nanobody comprises an amino acid sequence having less than 100% sequence identity to SEQ ID NO:187, then SEQ ID NO:187 has 100% sequence identity with the CDRs therein. In embodiments, SEQ ID NO: 187 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:188. In embodiments, the nanobody is as set forth in SEQ ID NO:188. In embodiments, the nanobody comprises an amino acid sequence with at least 85%, 86%, 87%, 88%, 89%, 90%, 188%, 92%, 93%, 94%, 95%, 96%, 97%, 98%, 99%, or 100% sequence identity to SEQ ID NO:188. In embodiments, the nanobody comprises an amino acid sequence with at least 85% sequence identity to SEQ ID NO:188. In embodiments, the nanobody comprises an amino acid sequence with at least 188% sequence identity to SEQ ID NO:188. In embodiments, the nanobody comprises an amino acid sequence with at least 92% sequence identity to SEQ ID NO:188. In embodiments, the nanobody comprises an amino acid sequence with at least 94% sequence identity to SEQ ID NO:188. In embodiments, the nanobody comprises an amino acid sequence with at least 95% sequence identity to SEQ ID NO:188. In embodiments, the nanobody comprises an amino acid sequence with at least 96% sequence identity to SEQ ID NO:188. In embodiments, the nanobody comprises an amino acid sequence with at least 98% sequence identity to SEQ ID NO:188. In embodiments, when the nanobody comprises an amino acid sequence having less than 100% sequence identity to SEQ ID NO:188, then SEQ ID NO:188 has 100% sequence identity with the CDRs therein. In embodiments, SEQ ID NO: 188 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:189. In embodiments, the nanobody is as set forth in SEQ ID NO:189. In embodiments, the nanobody comprises an amino acid sequence with at least 85%, 86%, 87%, 88%, 89%, 90%, 189%, 92%, 93%, 94%, 95%, 96%, 97%, 98%, 99%, or 100% sequence identity to SEQ ID NO:189. In embodiments, the nanobody comprises an amino acid sequence with at least 85% sequence identity to SEQ ID NO:189. In embodiments, the nanobody comprises an amino acid sequence with at least 189% sequence identity to SEQ ID NO:189. In embodiments, the nanobody comprises an amino acid sequence with at least 92% sequence identity to SEQ ID NO:189. In embodiments, the nanobody comprises an amino acid sequence with at least 94% sequence identity to SEQ ID NO:189. In embodiments, the nanobody comprises an amino acid sequence with at least 95% sequence identity to SEQ ID NO:189. In embodiments, the nanobody comprises an amino acid sequence with at least 96% sequence identity to SEQ ID NO:189. In embodiments, the nanobody comprises an amino acid sequence with at least 98% sequence identity to SEQ ID NO:189. In embodiments, when the nanobody comprises an amino acid sequence having less than 100% sequence identity to SEQ ID NO:189, then SEQ ID NO:189 has 100% sequence identity with the CDRs therein. In embodiments, SEQ ID NO: 189 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:201. In embodiments, the nanobody is as set forth in SEQ ID NO:201. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:201. In embodiments, the nanobody comprises an amino acid sequence with at least 85% sequence identity to SEQ ID NO:201. In embodiments, the nanobody comprises an amino acid sequence with at least 90% sequence identity to SEQ ID NO:201. In embodiments, the nanobody comprises an amino acid sequence with at least 92% sequence identity to SEQ ID NO:201. In embodiments, the nanobody comprises an amino acid sequence with at least 94% sequence identity to SEQ ID NO:201. In embodiments, the nanobody comprises an amino acid sequence with at least 95% sequence identity to SEQ ID NO:201. In embodiments, the nanobody comprises an amino acid sequence with at least 96% sequence identity to SEQ ID NO:201. In embodiments, the nanobody comprises an amino acid sequence with at least 98% sequence identity to SEQ ID NO:201. In embodiments, when the nanobody comprises an amino acid sequence having less than 100% sequence identity to SEQ ID NO:201, then SEQ ID NO:201 has 100% sequence identity with the CDRs therein. In embodiments, SEQ ID NO:201 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:203. In embodiments, the nanobody is as set forth in SEQ ID NO:203. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:203. In embodiments, the nanobody comprises an amino acid sequence with at least 85% sequence identity to SEQ ID NO:203. In embodiments, the nanobody comprises an amino acid sequence with at least 90% sequence identity to SEQ ID NO:203. In embodiments, the nanobody comprises an amino acid sequence with at least 92% sequence identity to SEQ ID NO:203. In embodiments, the nanobody comprises an amino acid sequence with at least 94% sequence identity to SEQ ID NO:203. In embodiments, the nanobody comprises an amino acid sequence with at least 95% sequence identity to SEQ ID NO:203. In embodiments, the nanobody comprises an amino acid sequence with at least 96% sequence identity to SEQ ID NO:203. In embodiments, the nanobody comprises an amino acid sequence with at least 98% sequence identity to SEQ ID NO:203. In embodiments, when the nanobody comprises an amino acid sequence having less than 100% sequence identity to SEQ ID NO:203, then SEQ ID NO:203 has 100% sequence identity with the CDRs therein. In embodiments, SEQ ID NO:203 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:205. In embodiments, the nanobody is as set forth in SEQ ID NO:205. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:205. In embodiments, the nanobody comprises an amino acid sequence with at least 85% sequence identity to SEQ ID NO:205. In embodiments, the nanobody comprises an amino acid sequence with at least 90% sequence identity to SEQ ID NO:205. In embodiments, the nanobody comprises an amino acid sequence with at least 92% sequence identity to SEQ ID NO:205. In embodiments, the nanobody comprises an amino acid sequence with at least 94% sequence identity to SEQ ID NO:205. In embodiments, the nanobody comprises an amino acid sequence with at least 95% sequence identity to SEQ ID NO:205. In embodiments, the nanobody comprises an amino acid sequence with at least 96% sequence identity to SEQ ID NO:205. In embodiments, the nanobody comprises an amino acid sequence with at least 98% sequence identity to SEQ ID NO:205. In embodiments, when the nanobody comprises an amino acid sequence having less than 100% sequence identity to SEQ ID NO:205, then SEQ ID NO:205 has 100% sequence identity with the CDRs therein. In embodiments, SEQ ID NO:205 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:207. In embodiments, the nanobody is as set forth in SEQ ID NO:207. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:207. In embodiments, the nanobody comprises an amino acid sequence with at least 85% sequence identity to SEQ ID NO:207. In embodiments, the nanobody comprises an amino acid sequence with at least 90% sequence identity to SEQ ID NO:207. In embodiments, the nanobody comprises an amino acid sequence with at least 92% sequence identity to SEQ ID NO:207. In embodiments, the nanobody comprises an amino acid sequence with at least 94% sequence identity to SEQ ID NO:207. In embodiments, the nanobody comprises an amino acid sequence with at least 95% sequence identity to SEQ ID NO:207. In embodiments, the nanobody comprises an amino acid sequence with at least 96% sequence identity to SEQ ID NO:207. In embodiments, the nanobody comprises an amino acid sequence with at least 98% sequence identity to SEQ ID NO:207. In embodiments, when the nanobody comprises an amino acid sequence having less than 100% sequence identity to SEQ ID NO:207, then SEQ ID NO:207 has 100% sequence identity with the CDRs therein. In embodiments, SEQ ID NO:207 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:209. In embodiments, the nanobody is as set forth in SEQ ID NO:209. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:209. In embodiments, the nanobody comprises an amino acid sequence with at least 85% sequence identity to SEQ ID NO:209. In embodiments, the nanobody comprises an amino acid sequence with at least 90% sequence identity to SEQ ID NO:209. In embodiments, the nanobody comprises an amino acid sequence with at least 92% sequence identity to SEQ ID NO:209. In embodiments, the nanobody comprises an amino acid sequence with at least 94% sequence identity to SEQ ID NO:209. In embodiments, the nanobody comprises an amino acid sequence with at least 95% sequence identity to SEQ ID NO:209. In embodiments, the nanobody comprises an amino acid sequence with at least 96% sequence identity to SEQ ID NO:209. In embodiments, the nanobody comprises an amino acid sequence with at least 98% sequence identity to SEQ ID NO:209. In embodiments, when the nanobody comprises an amino acid sequence having less than 100% sequence identity to SEQ ID NO:209, then SEQ ID NO:209 has 100% sequence identity with the CDRs therein. In embodiments, SEQ ID NO:209 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:211. In embodiments, the nanobody is as set forth in SEQ ID NO:211. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:211. In embodiments, the nanobody comprises an amino acid sequence with at least 85% sequence identity to SEQ ID NO:211. In embodiments, the nanobody comprises an amino acid sequence with at least 90% sequence identity to SEQ ID NO:211. In embodiments, the nanobody comprises an amino acid sequence with at least 92% sequence identity to SEQ ID NO:211. In embodiments, the nanobody comprises an amino acid sequence with at least 94% sequence identity to SEQ ID NO:211. In embodiments, the nanobody comprises an amino acid sequence with at least 95% sequence identity to SEQ ID NO:211. In embodiments, the nanobody comprises an amino acid sequence with at least 96% sequence identity to SEQ ID NO:211. In embodiments, the nanobody comprises an amino acid sequence with at least 98% sequence identity to SEQ ID NO:211. In embodiments, when the nanobody comprises an amino acid sequence having less than 100% sequence identity to SEQ ID NO:211, then SEQ ID NO:211 has 100% sequence identity with the CDRs therein. In embodiments, SEQ ID NO:211 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:227. In embodiments, the nanobody is as set forth in SEQ ID NO:227. In embodiments, the nanobody comprises an amino acid sequence with at least 8⁵%, 86%, 87%, 88%, 89%, 90%, 91%, 92%, 93%, 94%, 95%, 96%, 97%, 98%, 99%, or 100% sequence identity to SEQ ID NO:227. In embodiments, the nanobody comprises an amino acid sequence with at least 85% sequence identity to SEQ ID NO:227. In embodiments, the nanobody comprises an amino acid sequence with at least 90% sequence identity to SEQ ID NO:227. In embodiments, the nanobody comprises an amino acid sequence with at least 92% sequence identity to SEQ ID NO:227. In embodiments, the nanobody comprises an amino acid sequence with at least 94% sequence identity to SEQ ID NO:227. In embodiments, the nanobody comprises an amino acid sequence with at least 95% sequence identity to SEQ ID NO:227. In embodiments, the nanobody comprises an amino acid sequence with at least 96% sequence identity to SEQ ID NO:227. In embodiments, the nanobody comprises an amino acid sequence with at least 98% sequence identity to SEQ ID NO:227. In embodiments, when the nanobody comprises an amino acid sequence having less than 100% sequence identity to SEQ ID NO:227, then SEQ ID NO:227 has 100% sequence identity with the CDRs therein. In embodiments, SEQ ID NO:227 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:228. In embodiments, the nanobody is as set forth in SEQ ID NO:228. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:228. In embodiments, the nanobody comprises an amino acid sequence with at least 85% sequence identity to SEQ ID NO:228. In embodiments, the nanobody comprises an amino acid sequence with at least 90% sequence identity to SEQ ID NO:228. In embodiments, the nanobody comprises an amino acid sequence with at least 92% sequence identity to SEQ ID NO:228. In embodiments, the nanobody comprises an amino acid sequence with at least 94% sequence identity to SEQ ID NO:228. In embodiments, the nanobody comprises an amino acid sequence with at least 95% sequence identity to SEQ ID NO:228. In embodiments, the nanobody comprises an amino acid sequence with at least 96% sequence identity to SEQ ID NO:228. In embodiments, the nanobody comprises an amino acid sequence with at least 98% sequence identity to SEQ ID NO:228. In embodiments, when the nanobody comprises an amino acid sequence having less than 100% sequence identity to SEQ ID NO:228, then SEQ ID NO:228 has 100% sequence identity with the CDRs therein. In embodiments, SEQ ID NO:228 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:229. In embodiments, the nanobody is as set forth in SEQ ID NO:229. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:229. In embodiments, the nanobody comprises an amino acid sequence with at least 85% sequence identity to SEQ ID NO:229. In embodiments, the nanobody comprises an amino acid sequence with at least 90% sequence identity to SEQ ID NO:229. In embodiments, the nanobody comprises an amino acid sequence with at least 92% sequence identity to SEQ ID NO:229. In embodiments, the nanobody comprises an amino acid sequence with at least 94% sequence identity to SEQ ID NO:229. In embodiments, the nanobody comprises an amino acid sequence with at least 95% sequence identity to SEQ ID NO:229. In embodiments, the nanobody comprises an amino acid sequence with at least 96% sequence identity to SEQ ID NO:229. In embodiments, the nanobody comprises an amino acid sequence with at least 98% sequence identity to SEQ ID NO:229. In embodiments, when the nanobody comprises an amino acid sequence having less than 100% sequence identity to SEQ ID NO:229, then SEQ ID NO:229 has 100% sequence identity with the CDRs therein. In embodiments, SEQ ID NO:229 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:230. In embodiments, the nanobody is as set forth in SEQ ID NO:230. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:230. In embodiments, the nanobody comprises an amino acid sequence with at least 85% sequence identity to SEQ ID NO:230. In embodiments, the nanobody comprises an amino acid sequence with at least 90% sequence identity to SEQ ID NO:230. In embodiments, the nanobody comprises an amino acid sequence with at least 92% sequence identity to SEQ ID NO:230. In embodiments, the nanobody comprises an amino acid sequence with at least 94% sequence identity to SEQ ID NO:230. In embodiments, the nanobody comprises an amino acid sequence with at least 95% sequence identity to SEQ ID NO:230. In embodiments, the nanobody comprises an amino acid sequence with at least 96% sequence identity to SEQ ID NO:230. In embodiments, the nanobody comprises an amino acid sequence with at least 98% sequence identity to SEQ ID NO:230. In embodiments, when the nanobody comprises an amino acid sequence having less than 100% sequence identity to SEQ ID NO:230, then SEQ ID NO:230 has 100% sequence identity with the CDRs therein. In embodiments, SEQ ID NO:230 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:231. In embodiments, the nanobody is as set forth in SEQ ID NO:231. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:231. In embodiments, the nanobody comprises an amino acid sequence with at least 85% sequence identity to SEQ ID NO:231. In embodiments, the nanobody comprises an amino acid sequence with at least 90% sequence identity to SEQ ID NO:231. In embodiments, the nanobody comprises an amino acid sequence with at least 92% sequence identity to SEQ ID NO:231. In embodiments, the nanobody comprises an amino acid sequence with at least 94% sequence identity to SEQ ID NO:231. In embodiments, the nanobody comprises an amino acid sequence with at least 95% sequence identity to SEQ ID NO:231. In embodiments, the nanobody comprises an amino acid sequence with at least 96% sequence identity to SEQ ID NO:231. In embodiments, the nanobody comprises an amino acid sequence with at least 98% sequence identity to SEQ ID NO:231. In embodiments, when the nanobody comprises an amino acid sequence having less than 100% sequence identity to SEQ ID NO:231, then SEQ ID NO:231 has 100% sequence identity with the CDRs therein. In embodiments, SEQ ID NO:231 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:232. In embodiments, the nanobody is as set forth in SEQ ID NO:232. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:232. In embodiments, the nanobody comprises an amino acid sequence with at least 85% sequence identity to SEQ ID NO:232. In embodiments, the nanobody comprises an amino acid sequence with at least 90% sequence identity to SEQ ID NO:232. In embodiments, the nanobody comprises an amino acid sequence with at least 92% sequence identity to SEQ ID NO:232. In embodiments, the nanobody comprises an amino acid sequence with at least 94% sequence identity to SEQ ID NO:232. In embodiments, the nanobody comprises an amino acid sequence with at least 95% sequence identity to SEQ ID NO:232. In embodiments, the nanobody comprises an amino acid sequence with at least 96% sequence identity to SEQ ID NO:232. In embodiments, the nanobody comprises an amino acid sequence with at least 98% sequence identity to SEQ ID NO:232. In embodiments, when the nanobody comprises an amino acid sequence having less than 100% sequence identity to SEQ ID NO:232, then SEQ ID NO:232 has 100% sequence identity with the CDRs therein. In embodiments, SEQ ID NO:232 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:233. In embodiments, the nanobody is as set forth in SEQ ID NO:233. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:233. In embodiments, the nanobody comprises an amino acid sequence with at least 85% sequence identity to SEQ ID NO:233. In embodiments, the nanobody comprises an amino acid sequence with at least 90% sequence identity to SEQ ID NO:233. In embodiments, the nanobody comprises an amino acid sequence with at least 92% sequence identity to SEQ ID NO:233. In embodiments, the nanobody comprises an amino acid sequence with at least 94% sequence identity to SEQ ID NO:233. In embodiments, the nanobody comprises an amino acid sequence with at least 95% sequence identity to SEQ ID NO:233. In embodiments, the nanobody comprises an amino acid sequence with at least 96% sequence identity to SEQ ID NO:233. In embodiments, the nanobody comprises an amino acid sequence with at least 98% sequence identity to SEQ ID NO:233. In embodiments, when the nanobody comprises an amino acid sequence having less than 100% sequence identity to SEQ ID NO:233, then SEQ ID NO:233 has 100% sequence identity with the CDRs therein. In embodiments, SEQ ID NO:233 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:234. In embodiments, the nanobody is as set forth in SEQ ID NO:234. In embodiments, the nanobody comprises an amino acid sequence with at least 8⁵%, 86%, 87%, 88%, 89%, 90%, 91%, 92%, 93%, 94%, 95%, 96%, 97%, 98%, 99%, or 100% sequence identity to SEQ ID NO:234. In embodiments, the nanobody comprises an amino acid sequence with at least 85% sequence identity to SEQ ID NO:234. In embodiments, the nanobody comprises an amino acid sequence with at least 90% sequence identity to SEQ ID NO:234. In embodiments, the nanobody comprises an amino acid sequence with at least 92% sequence identity to SEQ ID NO:234. In embodiments, the nanobody comprises an amino acid sequence with at least 94% sequence identity to SEQ ID NO:234. In embodiments, the nanobody comprises an amino acid sequence with at least 95% sequence identity to SEQ ID NO:234. In embodiments, the nanobody comprises an amino acid sequence with at least 96% sequence identity to SEQ ID NO:234. In embodiments, the nanobody comprises an amino acid sequence with at least 98% sequence identity to SEQ ID NO:234. In embodiments, when the nanobody comprises an amino acid sequence having less than 100% sequence identity to SEQ ID NO:234, then SEQ ID NO:234 has 100% sequence identity with the CDRs therein. In embodiments, SEQ ID NO:234 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:235. In embodiments, the nanobody is as set forth in SEQ ID NO:235. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:235. In embodiments, the nanobody comprises an amino acid sequence with at least 85% sequence identity to SEQ ID NO:235. In embodiments, the nanobody comprises an amino acid sequence with at least 90% sequence identity to SEQ ID NO:235. In embodiments, the nanobody comprises an amino acid sequence with at least 92% sequence identity to SEQ ID NO:235. In embodiments, the nanobody comprises an amino acid sequence with at least 94% sequence identity to SEQ ID NO:235. In embodiments, the nanobody comprises an amino acid sequence with at least 95% sequence identity to SEQ ID NO:235. In embodiments, the nanobody comprises an amino acid sequence with at least 96% sequence identity to SEQ ID NO:235. In embodiments, the nanobody comprises an amino acid sequence with at least 98% sequence identity to SEQ ID NO:235. In embodiments, when the nanobody comprises an amino acid sequence having less than 100% sequence identity to SEQ ID NO:235, then SEQ ID NO:235 has 100% sequence identity with the CDRs therein. In embodiments, SEQ ID NO:235 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:236. In embodiments, the nanobody is as set forth in SEQ ID NO:236. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:236. In embodiments, the nanobody comprises an amino acid sequence with at least 85% sequence identity to SEQ ID NO:236. In embodiments, the nanobody comprises an amino acid sequence with at least 90% sequence identity to SEQ ID NO:236. In embodiments, the nanobody comprises an amino acid sequence with at least 92% sequence identity to SEQ ID NO:236. In embodiments, the nanobody comprises an amino acid sequence with at least 94% sequence identity to SEQ ID NO:236. In embodiments, the nanobody comprises an amino acid sequence with at least 95% sequence identity to SEQ ID NO:236. In embodiments, the nanobody comprises an amino acid sequence with at least 96% sequence identity to SEQ ID NO:236. In embodiments, the nanobody comprises an amino acid sequence with at least 98% sequence identity to SEQ ID NO:236. In embodiments, when the nanobody comprises an amino acid sequence having less than 100% sequence identity to SEQ ID NO:236, then SEQ ID NO:236 has 100% sequence identity with the CDRs therein. In embodiments, SEQ ID NO:236 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:237. In embodiments, the nanobody is as set forth in SEQ ID NO:237. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:237. In embodiments, the nanobody comprises an amino acid sequence with at least 85% sequence identity to SEQ ID NO:237. In embodiments, the nanobody comprises an amino acid sequence with at least 90% sequence identity to SEQ ID NO:237. In embodiments, the nanobody comprises an amino acid sequence with at least 92% sequence identity to SEQ ID NO:237. In embodiments, the nanobody comprises an amino acid sequence with at least 94% sequence identity to SEQ ID NO:237. In embodiments, the nanobody comprises an amino acid sequence with at least 95% sequence identity to SEQ ID NO:237. In embodiments, the nanobody comprises an amino acid sequence with at least 96% sequence identity to SEQ ID NO:237. In embodiments, the nanobody comprises an amino acid sequence with at least 98% sequence identity to SEQ ID NO:237. In embodiments, when the nanobody comprises an amino acid sequence having less than 100% sequence identity to SEQ ID NO:237, then SEQ ID NO:237 has 100% sequence identity with the CDRs therein. In embodiments, SEQ ID NO:237 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:238. In embodiments, the nanobody is as set forth in SEQ ID NO:238. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:238. In embodiments, the nanobody comprises an amino acid sequence with at least 85% sequence identity to SEQ ID NO:238. In embodiments, the nanobody comprises an amino acid sequence with at least 90% sequence identity to SEQ ID NO:238. In embodiments, the nanobody comprises an amino acid sequence with at least 92% sequence identity to SEQ ID NO:238. In embodiments, the nanobody comprises an amino acid sequence with at least 94% sequence identity to SEQ ID NO:238. In embodiments, the nanobody comprises an amino acid sequence with at least 95% sequence identity to SEQ ID NO:238. In embodiments, the nanobody comprises an amino acid sequence with at least 96% sequence identity to SEQ ID NO:238. In embodiments, the nanobody comprises an amino acid sequence with at least 98% sequence identity to SEQ ID NO:238. In embodiments, when the nanobody comprises an amino acid sequence having less than 100% sequence identity to SEQ ID NO:238, then SEQ ID NO:238 has 100% sequence identity with the CDRs therein. In embodiments, SEQ ID NO:238 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the disclosure provides a biomolecule conjugate comprising any one of SEQ ID NOS:227-238 as described herein, including embodiments thereof, covalently bonded to mesothelin (MSLN). In embodiments, the biomolecule conjugate comprises any one of SEQ ID NOS:227-238 as described herein, including embodiments thereof, covalently bonded to MSLN expressed on a cancer tumor. In embodiments, the biomolecule conjugate comprises any one of SEQ ID NOS:227-238 as described herein, including embodiments thereof, covalently bonded to MSLN overexpressed on a cancer tumor.

In embodiments, the nanobody comprises the amino acid sequence of SEQ ID NO:255. In embodiments, the nanobody is as set forth in SEQ ID NO:255. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:255. In embodiments, the nanobody comprises an amino acid sequence with at least 85% sequence identity to SEQ ID NO:255. In embodiments, the nanobody comprises an amino acid sequence with at least 90% sequence identity to SEQ ID NO:255. In embodiments, the nanobody comprises an amino acid sequence with at least 92% sequence identity to SEQ ID NO:255. In embodiments, the nanobody comprises an amino acid sequence with at least 94% sequence identity to SEQ ID NO:255. In embodiments, the nanobody comprises an amino acid sequence with at least 95% sequence identity to SEQ ID NO:255. In embodiments, the nanobody comprises an amino acid sequence with at least 96% sequence identity to SEQ ID NO:255. In embodiments, the nanobody comprises an amino acid sequence with at least 98% sequence identity to SEQ ID NO:255. In embodiments, when the nanobody comprises an amino acid sequence having less than 100% sequence identity to SEQ ID NO:255, then SEQ ID NO:255 has 100% sequence identity with the CDRs therein. In embodiments, SEQ ID NO:255 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:256. In embodiments, the nanobody is as set forth in SEQ ID NO:256. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:256. In embodiments, the nanobody comprises an amino acid sequence with at least 85% sequence identity to SEQ ID NO:256. In embodiments, the nanobody comprises an amino acid sequence with at least 90% sequence identity to SEQ ID NO:256. In embodiments, the nanobody comprises an amino acid sequence with at least 92% sequence identity to SEQ ID NO:256. In embodiments, the nanobody comprises an amino acid sequence with at least 94% sequence identity to SEQ ID NO:256. In embodiments, the nanobody comprises an amino acid sequence with at least 95% sequence identity to SEQ ID NO:256. In embodiments, the nanobody comprises an amino acid sequence with at least 96% sequence identity to SEQ ID NO:256. In embodiments, the nanobody comprises an amino acid sequence with at least 98% sequence identity to SEQ ID NO:256. In embodiments, when the nanobody comprises an amino acid sequence having less than 100% sequence identity to SEQ ID NO:256, then SEQ ID NO:256 has 100% sequence identity with the CDRs therein. In embodiments, SEQ ID NO:256 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:257. In embodiments, the nanobody is as set forth in SEQ ID NO:257. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:257. In embodiments, the nanobody comprises an amino acid sequence with at least 8⁵% sequence identity to SEQ ID NO:257. In embodiments, the nanobody comprises an amino acid sequence with at least 90% sequence identity to SEQ ID NO:257. In embodiments, the nanobody comprises an amino acid sequence with at least 92% sequence identity to SEQ ID NO:257. In embodiments, the nanobody comprises an amino acid sequence with at least 94% sequence identity to SEQ ID NO:257. In embodiments, the nanobody comprises an amino acid sequence with at least 95% sequence identity to SEQ ID NO:257. In embodiments, the nanobody comprises an amino acid sequence with at least 96% sequence identity to SEQ ID NO:257. In embodiments, the nanobody comprises an amino acid sequence with at least 98% sequence identity to SEQ ID NO:257. In embodiments, when the nanobody comprises an amino acid sequence having less than 100% sequence identity to SEQ ID NO:257, then SEQ ID NO:257 has 100% sequence identity with the CDRs therein. In embodiments, SEQ ID NO:257 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:258. In embodiments, the nanobody is as set forth in SEQ ID NO:258. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:258. In embodiments, the nanobody comprises an amino acid sequence with at least 85% sequence identity to SEQ ID NO:258. In embodiments, the nanobody comprises an amino acid sequence with at least 90% sequence identity to SEQ ID NO:258. In embodiments, the nanobody comprises an amino acid sequence with at least 92% sequence identity to SEQ ID NO:258. In embodiments, the nanobody comprises an amino acid sequence with at least 94% sequence identity to SEQ ID NO:258. In embodiments, the nanobody comprises an amino acid sequence with at least 95% sequence identity to SEQ ID NO:258. In embodiments, the nanobody comprises an amino acid sequence with at least 96% sequence identity to SEQ ID NO:258. In embodiments, the nanobody comprises an amino acid sequence with at least 98% sequence identity to SEQ ID NO:258. In embodiments, when the nanobody comprises an amino acid sequence having less than 100% sequence identity to SEQ ID NO:258, then SEQ ID NO:258 has 100% sequence identity with the CDRs therein. In embodiments, SEQ ID NO:258 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:259. In embodiments, the nanobody is as set forth in SEQ ID NO:259. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:259. In embodiments, the nanobody comprises an amino acid sequence with at least 85% sequence identity to SEQ ID NO:259. In embodiments, the nanobody comprises an amino acid sequence with at least 90% sequence identity to SEQ ID NO:259. In embodiments, the nanobody comprises an amino acid sequence with at least 92% sequence identity to SEQ ID NO:259. In embodiments, the nanobody comprises an amino acid sequence with at least 94% sequence identity to SEQ ID NO:259. In embodiments, the nanobody comprises an amino acid sequence with at least 95% sequence identity to SEQ ID NO:259. In embodiments, the nanobody comprises an amino acid sequence with at least 96% sequence identity to SEQ ID NO:259. In embodiments, the nanobody comprises an amino acid sequence with at least 98% sequence identity to SEQ ID NO:259. In embodiments, when the nanobody comprises an amino acid sequence having less than 100% sequence identity to SEQ ID NO:259, then SEQ ID NO:259 has 100% sequence identity with the CDRs therein. In embodiments, SEQ ID NO:259 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:260. In embodiments, the nanobody is as set forth in SEQ ID NO:260. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:260. In embodiments, the nanobody comprises an amino acid sequence with at least 85% sequence identity to SEQ ID NO:260. In embodiments, the nanobody comprises an amino acid sequence with at least 90% sequence identity to SEQ ID NO:260. In embodiments, the nanobody comprises an amino acid sequence with at least 92% sequence identity to SEQ ID NO:260. In embodiments, the nanobody comprises an amino acid sequence with at least 94% sequence identity to SEQ ID NO:260. In embodiments, the nanobody comprises an amino acid sequence with at least 95% sequence identity to SEQ ID NO:260. In embodiments, the nanobody comprises an amino acid sequence with at least 96% sequence identity to SEQ ID NO:260. In embodiments, the nanobody comprises an amino acid sequence with at least 98% sequence identity to SEQ ID NO:260. In embodiments, when the nanobody comprises an amino acid sequence having less than 100% sequence identity to SEQ ID NO:260, then SEQ ID NO:260 has 100% sequence identity with the CDRs therein. In embodiments, SEQ ID NO:260 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:261. In embodiments, the nanobody is as set forth in SEQ ID NO:261. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:261. In embodiments, the nanobody comprises an amino acid sequence with at least 85% sequence identity to SEQ ID NO:261. In embodiments, the nanobody comprises an amino acid sequence with at least 90% sequence identity to SEQ ID NO:261. In embodiments, the nanobody comprises an amino acid sequence with at least 92% sequence identity to SEQ ID NO:261. In embodiments, the nanobody comprises an amino acid sequence with at least 94% sequence identity to SEQ ID NO:261. In embodiments, the nanobody comprises an amino acid sequence with at least 95% sequence identity to SEQ ID NO:261. In embodiments, the nanobody comprises an amino acid sequence with at least 96% sequence identity to SEQ ID NO:261. In embodiments, the nanobody comprises an amino acid sequence with at least 98% sequence identity to SEQ ID NO:261. In embodiments, when the nanobody comprises an amino acid sequence having less than 100% sequence identity to SEQ ID NO:261, then SEQ ID NO:261 has 100% sequence identity with the CDRs therein. In embodiments, SEQ ID NO:261 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:262. In embodiments, the nanobody is as set forth in SEQ ID NO:262. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:262. In embodiments, the nanobody comprises an amino acid sequence with at least 85% sequence identity to SEQ ID NO:262. In embodiments, the nanobody comprises an amino acid sequence with at least 90% sequence identity to SEQ ID NO:262. In embodiments, the nanobody comprises an amino acid sequence with at least 92% sequence identity to SEQ ID NO:262. In embodiments, the nanobody comprises an amino acid sequence with at least 94% sequence identity to SEQ ID NO:262. In embodiments, the nanobody comprises an amino acid sequence with at least 95% sequence identity to SEQ ID NO:262. In embodiments, the nanobody comprises an amino acid sequence with at least 96% sequence identity to SEQ ID NO:262. In embodiments, the nanobody comprises an amino acid sequence with at least 98% sequence identity to SEQ ID NO:262. In embodiments, when the nanobody comprises an amino acid sequence having less than 100% sequence identity to SEQ ID NO:262, then SEQ ID NO:262 has 100% sequence identity with the CDRs therein. In embodiments, SEQ ID NO:262 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:263. In embodiments, the nanobody is as set forth in SEQ ID NO:263. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:263. In embodiments, the nanobody comprises an amino acid sequence with at least 85% sequence identity to SEQ ID NO:263. In embodiments, the nanobody comprises an amino acid sequence with at least 90% sequence identity to SEQ ID NO:263. In embodiments, the nanobody comprises an amino acid sequence with at least 92% sequence identity to SEQ ID NO:263. In embodiments, the nanobody comprises an amino acid sequence with at least 94% sequence identity to SEQ ID NO:263. In embodiments, the nanobody comprises an amino acid sequence with at least 95% sequence identity to SEQ ID NO:263. In embodiments, the nanobody comprises an amino acid sequence with at least 96% sequence identity to SEQ ID NO:263. In embodiments, the nanobody comprises an amino acid sequence with at least 98% sequence identity to SEQ ID NO:263. In embodiments, when the nanobody comprises an amino acid sequence having less than 100% sequence identity to SEQ ID NO:263, then SEQ ID NO:263 has 100% sequence identity with the CDRs therein. In embodiments, SEQ ID NO:263 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:264. In embodiments, the nanobody is as set forth in SEQ ID NO:264. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:264. In embodiments, the nanobody comprises an amino acid sequence with at least 8⁵% sequence identity to SEQ ID NO:264. In embodiments, the nanobody comprises an amino acid sequence with at least 90% sequence identity to SEQ ID NO:264. In embodiments, the nanobody comprises an amino acid sequence with at least 92% sequence identity to SEQ ID NO:264. In embodiments, the nanobody comprises an amino acid sequence with at least 94% sequence identity to SEQ ID NO:264. In embodiments, the nanobody comprises an amino acid sequence with at least 95% sequence identity to SEQ ID NO:264. In embodiments, the nanobody comprises an amino acid sequence with at least 96% sequence identity to SEQ ID NO:264. In embodiments, the nanobody comprises an amino acid sequence with at least 98% sequence identity to SEQ ID NO:264. In embodiments, when the nanobody comprises an amino acid sequence having less than 100% sequence identity to SEQ ID NO:264, then SEQ ID NO:264 has 100% sequence identity with the CDRs therein. In embodiments, SEQ ID NO:264 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:265. In embodiments, the nanobody is as set forth in SEQ ID NO:265. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:265. In embodiments, the nanobody comprises an amino acid sequence with at least 85% sequence identity to SEQ ID NO:265. In embodiments, the nanobody comprises an amino acid sequence with at least 90% sequence identity to SEQ ID NO:265. In embodiments, the nanobody comprises an amino acid sequence with at least 92% sequence identity to SEQ ID NO:265. In embodiments, the nanobody comprises an amino acid sequence with at least 94% sequence identity to SEQ ID NO:265. In embodiments, the nanobody comprises an amino acid sequence with at least 95% sequence identity to SEQ ID NO:265. In embodiments, the nanobody comprises an amino acid sequence with at least 96% sequence identity to SEQ ID NO:265. In embodiments, the nanobody comprises an amino acid sequence with at least 98% sequence identity to SEQ ID NO:265. In embodiments, when the nanobody comprises an amino acid sequence having less than 100% sequence identity to SEQ ID NO:265, then SEQ ID NO:265 has 100% sequence identity with the CDRs therein. In embodiments, SEQ ID NO:265 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:266. In embodiments, the nanobody is as set forth in SEQ ID NO:266. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:266. In embodiments, the nanobody comprises an amino acid sequence with at least 85% sequence identity to SEQ ID NO:266. In embodiments, the nanobody comprises an amino acid sequence with at least 90% sequence identity to SEQ ID NO:266. In embodiments, the nanobody comprises an amino acid sequence with at least 92% sequence identity to SEQ ID NO:266. In embodiments, the nanobody comprises an amino acid sequence with at least 94% sequence identity to SEQ ID NO:266. In embodiments, the nanobody comprises an amino acid sequence with at least 95% sequence identity to SEQ ID NO:266. In embodiments, the nanobody comprises an amino acid sequence with at least 96% sequence identity to SEQ ID NO:266. In embodiments, the nanobody comprises an amino acid sequence with at least 98% sequence identity to SEQ ID NO:266. In embodiments, when the nanobody comprises an amino acid sequence having less than 100% sequence identity to SEQ ID NO:266, then SEQ ID NO:266 has 100% sequence identity with the CDRs therein. In embodiments, SEQ ID NO:266 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the disclosure provides a biomolecule conjugate comprising any one of SEQ ID NOS:255-266 as described herein, including embodiments thereof, covalently bonded to MSLN. In embodiments, the biomolecule conjugate comprises any one of SEQ ID NOS:255-266 as described herein, including embodiments thereof, covalently bonded to MSLN expressed on a cancer tumor. In embodiments, the biomolecule conjugate comprises any one of SEQ ID NOS:255-266 as described herein, including embodiments thereof, covalently bonded to MSLN overexpressed on a cancer tumor.

In embodiments, the nanobody comprises the amino acid sequence of SEQ ID NO:267. In embodiments, the nanobody is as set forth in SEQ ID NO:267. In embodiments, the nanobody comprises an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:267. In embodiments, the nanobody comprises an amino acid sequence with at least 85% sequence identity to SEQ ID NO:267. In embodiments, the nanobody comprises an amino acid sequence with at least 90% sequence identity to SEQ ID NO:267. In embodiments, the nanobody comprises an amino acid sequence with at least 92% sequence identity to SEQ ID NO:267. In embodiments, the nanobody comprises an amino acid sequence with at least 94% sequence identity to SEQ ID NO:267. In embodiments, the nanobody comprises an amino acid sequence with at least 95% sequence identity to SEQ ID NO:267. In embodiments, the nanobody comprises an amino acid sequence with at least 96% sequence identity to SEQ ID NO:267. In embodiments, the nanobody comprises an amino acid sequence with at least 98% sequence identity to SEQ ID NO:267. In embodiments, when the nanobody comprises an amino acid sequence having less than 100% sequence identity to SEQ ID NO:267, then SEQ ID NO:267 has 100% sequence identity with the CDRs therein. In embodiments, SEQ ID NO:267 further comprises a His6-tag at the C-terminus. In embodiments, the nanobody further comprises a detectable agent. In embodiments, the nanobody further comprises a radioisotope. In embodiments, the nanobody further comprises a therapeutic agent. In embodiments, the nanobody further comprises a detectable agent and a therapeutic agent.

In embodiments, the nanobody comprises the amino acid sequence of SEQ ID NO:179. In embodiments, the nanobody is as set forth in SEQ ID NO:179. In embodiments, the nanobody comprises an amino acid sequence with at least 85%, 86%, 87%, 88%, 89%, 90%, 189%, 92%, 93%, 94%, 95%, 96%, 97%, 98%, 99%, sequence identity to SEQ ID NO:179, provided that the amino acid at the position corresponding to position 108 in SEQ ID NO:179 is meta-FSY. In embodiments, the nanobody comprises the amino acid sequence of SEQ ID NO:178, wherein one amino acid selected from the group consisting of E102, D103, P104, T105, T107, L108, V109, T110, S111, S112, and G113 is replaced by meta-FSY. In embodiments, the nanobody comprises an amino acid sequence with at least 85%, 86%, 87%, 88%, 89%, 90%, 189%, 92%, 93%, 94%, 95%, 96%, 97%, 98%, 99%, sequence identity to SEQ ID NO:178, provided that one amino acid selected from the group consisting of E102, D103, P104, T105, T107, L108, V109, T110, S111, S112, and G113 is replaced by meta-FSY.

Provided herein are fusion proteins. In embodiments, the fusion protein comprises a first protein and a second protein, wherein the first protein is a nanobody as described herein, including embodiments thereof. In embodiments, the fusion protein comprises a first protein and a second protein, wherein the first protein is a nanobody as described herein, including embodiments thereof, wherein the first protein is covalently bonded to the second protein via a glycine-serine peptide linker. In embodiments, the fusion protein comprises a first protein and a second protein, wherein the first protein is a nanobody as described herein, including embodiments thereof, and wherein the second protein is an antigen-binding fragment, a single-chain variable fragment, a second nanobody, or an affibody. In embodiments, the second protein is an antigen-binding fragment. In embodiments, the second protein is a single-chain variable fragment. In embodiments, the second protein is a second nanobody, wherein the second nanobody is different from the first nanobody. In embodiments, the second protein is a second nanobody, wherein the second nanobody is the same as the first nanobody. In embodiments, the second protein is an affibody. In embodiments, the second protein is an antibody. In embodiments, the fusion protein further comprises a third protein, wherein the third protein is an antigen-binding fragment, a single-chain variable fragment, a second nanobody, or an affibody. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the first protein is covalently bonded to the second protein via a glycine-serine peptide linker. Any glycine-serine peptide linker known in the art can be used to covalently bond the proteins. In embodiments, the glycine-serine peptide linker consists of 1 to 20 amino acids consisting of glycine and serine. In embodiments, the glycine-serine peptide linker consists of 2 to 12 amino acids consisting of glycine and serine. In embodiments, the glycine-serine peptide linker consists of 4 to 12 amino acids consisting of glycine and serine. In embodiments, the glycine-serine peptide linker has the formula -(G_{b}S)_{c}(G_{d}S)ₑ—, wherein “G” is glycine, “S” is serine, and wherein b and d are each independently an integer from 1 to 8, c is an integer from 0 to 4, and d is an integer from 1 to 8. In embodiments, b is an integer from 2 to 4, d is an integer from 2 to 6, and c is 0 or 1, and e is an integer from 1 to 4. In embodiments, the glycine-serine peptide linker has the formula -(G_{b}S)_{c}(G_{d}S)ₑG-, wherein b, c, d, and e are as defined herein. In embodiments, the glycine-serine peptide linker is SEQ ID NO:190. In embodiments, the glycine-serine peptide linker is SEQ ID NO:191.

In embodiments, the first protein comprises CDR1 as set forth in SEQ ID NO:155, CDR2 as set forth in SEQ ID NO:156, and CDR3 as set forth in SEQ ID NO:158 or 159. In embodiments, the first protein comprises CDR1 as set forth in SEQ ID NO:155, CDR2 as set forth in SEQ ID NO: 156, and CDR3 as set forth in SEQ ID NO:183 or 184. In embodiments, the first protein comprises CDR1 as set forth in SEQ ID NO:93, CDR2 as set forth in any one of SEQ ID NOS:96-102 and 105-113, and CDR3 as set forth in SEQ ID NO:95. In embodiments, the first protein comprises CDR1 as set forth in SEQ ID NO:93, CDR2 as set forth in SEQ ID NO:94, and CDR3 as set forth in SEQ ID NO: 103, 104, 114, or 115. In embodiments, the first protein comprises CDR1 as set forth in SEQ ID NO:67, CDR2 as set forth in SEQ ID NO:70, and CDR3 as set forth in SEQ ID NO:69. In embodiments, the first protein comprises CDR1 as set forth in SEQ ID NO:67, CDR2 as set forth in SEQ ID NO:68, and CDR3 as set forth in SEQ ID NO:71.

In embodiments, the fusion protein comprises a first protein and a second protein, wherein the first protein is a nanobody as described herein, including embodiments thereof, and wherein the second protein has at least 90% sequence identity to the amino acid sequence of SEQ ID NO:219 (MS211), SEQ ID NO:137 (ZHER2:2891), SEQ ID NO:138 (ZHER2:342), or SEQ ID NO:139 (F57). In embodiments, the fusion protein comprises a first protein and a second protein, wherein the first protein is a nanobody as described herein, including embodiments thereof, and wherein the second protein has at least 95% sequence identity to the amino acid sequence of SEQ ID NO:219 (MS211), SEQ ID NO:137 (ZHER2:2891), SEQ ID NO:138 (ZHER2:342), or SEQ ID NO:139 (F57). In embodiments, the fusion protein comprises a first protein and a second protein, wherein the first protein is a nanobody as described herein, including embodiments thereof, and wherein the second protein is as set forth in SEQ ID NO:219 (MS211), SEQ ID NO:137 (ZHER2:2891), SEQ ID NO:138 (ZHER2:342), or SEQ ID NO:139 (F57). In embodiments, the second protein is SEQ ID NO:219, including those having 85% 90%, 92%, 94%, 95%, 96%, 98%, and 100% sequence identity thereto. In embodiments, the second protein has at least 90% sequence identity to SEQ ID NO:219. In embodiments, the second protein has at least 95% sequence identity to SEQ ID NO:219. In embodiments, the second protein comprises sequence identity to SEQ ID NO:219. In embodiments, the second protein is SEQ ID NO:137, including those having 85% 90%, 92%, 94%, 95%, 96%, 98%, and 100% sequence identity thereto. In embodiments, the second protein has at least 90% sequence identity to SEQ ID NO:137. In embodiments, the second protein has at least 95% sequence identity to SEQ ID NO: 137. In embodiments, the second protein comprises sequence identity to SEQ ID NO:137. In embodiments, the second protein is SEQ ID NO:138, including those having 85% 90%, 92%, 94%, 95%, 96%, 98%, and 100% sequence identity thereto. In embodiments, the second protein has at least 90% sequence identity to SEQ ID NO:138. In embodiments, the second protein has at least 95% sequence identity to SEQ ID NO:138. In embodiments, the second protein comprises sequence identity to SEQ ID NO:138. In embodiments, the second protein is SEQ ID NO:139, including those having 85% 90%, 92%, 94%, 95%, 96%, 98%, and 100% sequence identity thereto. In embodiments, the second protein has at least 90% sequence identity to SEQ ID NO:139. In embodiments, the second protein has at least 95% sequence identity to SEQ ID NO:139. In embodiments, the second protein comprises sequence identity to SEQ ID NO: 139.

In embodiments, the fusion protein comprises a first nanobody and a second nanobody, wherein the first nanobody comprises (i) CDR1 as set forth in SEQ ID NO:155, CDR2 as set forth in SEQ ID NO: 156, and CDR3 as set forth in SEQ ID NO:157; or (ii) CDR1 as set forth in SEQ ID NO:155, CDR2 as set forth in SEQ ID NO:156, and CDR3 as set forth in SEQ ID NO:158 or 159; and wherein the second protein comprises a second nanobody, wherein the second nanobody comprises: (a) CDR1 as set forth in SEQ ID NO:67, CDR2 as set forth in SEQ ID NO:68, and CDR3 as set forth in SEQ ID NO:69; (b) CDR1 as set forth in SEQ ID NO:67, CDR2 as set forth in SEQ ID NO:70, and CDR3 as set forth in SEQ ID NO:69; or (c) CDR1 as set forth in SEQ ID NO:67, CDR2 as set forth in SEQ ID NO:68, and CDR3 as set forth in SEQ ID NO:71; provided that the first nanobody is not (i) when the second nanobody is (a). In embodiments, the fusion protein comprises a first nanobody and a second nanobody, wherein the first nanobody comprises CDR1 as set forth in SEQ ID NO:155, CDR2 as set forth in SEQ ID NO:156, and CDR3 as set forth in SEQ ID NO:157, and the second nanobody comprises CDR1 as set forth in SEQ ID NO:67, CDR2 as set forth in SEQ ID NO:70, and CDR3 as set forth in SEQ ID NO:69. In embodiments, the fusion protein comprises a first nanobody and a second nanobody, wherein the first nanobody comprises CDR1 as set forth in SEQ ID NO:155, CDR2 as set forth in SEQ ID NO:156, and CDR3 as set forth in SEQ ID NO:157, and the second nanobody comprises CDR1 as set forth in SEQ ID NO:67, CDR2 as set forth in SEQ ID NO:68, and CDR3 as set forth in SEQ ID NO:71. In embodiments, the fusion protein comprises a first nanobody and a second nanobody, wherein the first nanobody comprises CDR1 as set forth in SEQ ID NO:67, CDR2 as set forth in SEQ ID NO:68, and CDR3 as set forth in SEQ ID NO:69, and the second nanobody comprises CDR1 as set forth in SEQ ID NO: 155, CDR2 as set forth in SEQ ID NO:156, and CDR3 as set forth in SEQ ID NO:158. In embodiments, the fusion protein comprises a first nanobody and a second nanobody, wherein the first nanobody comprises CDR1 as set forth in SEQ ID NO:67, CDR2 as set forth in SEQ ID NO:68, and CDR3 as set forth in SEQ ID NO:69, and the second nanobody comprises CDR1 as set forth in SEQ ID NO:155, CDR2 as set forth in SEQ ID NO:156, and CDR3 as set forth in SEQ ID NO:159. In embodiments, the fusion protein comprises a first nanobody and a second nanobody, wherein the first nanobody comprises CDR1 as set forth in SEQ ID NO:155, CDR2 as set forth in SEQ ID NO:156, and CDR3 as set forth in SEQ ID NO:158, and the second nanobody comprises CDR1 as set forth in SEQ ID NO:67, CDR2 as set forth in SEQ ID NO:70, and CDR3 as set forth in SEQ ID NO:69. In embodiments, the fusion protein comprises a first nanobody and a second nanobody, wherein the first nanobody comprises CDR1 as set forth in SEQ ID NO:155, CDR2 as set forth in SEQ ID NO:156, and CDR3 as set forth in SEQ ID NO:158, and the second nanobody comprises CDR1 as set forth in SEQ ID NO:67, CDR2 as set forth in SEQ ID NO:70, and CDR3 as set forth in SEQ ID NO:69. In embodiments, the fusion protein comprises a first nanobody and a second nanobody, wherein the first nanobody comprises CDR1 as set forth in SEQ ID NO:155, CDR2 as set forth in SEQ ID NO:156, and CDR3 as set forth in SEQ ID NO:159, and the second nanobody comprises CDR1 as set forth in SEQ ID NO:67, CDR2 as set forth in SEQ ID NO:70, and CDR3 as set forth in SEQ ID NO:69. In embodiments, the fusion protein comprises a first nanobody and a second nanobody, wherein the first nanobody comprises CDR1 as set forth in SEQ ID NO:155, CDR2 as set forth in SEQ ID NO:156, and CDR3 as set forth in SEQ ID NO: 159, and the second nanobody comprises CDR1 as set forth in SEQ ID NO:67, CDR2 as set forth in SEQ ID NO:68, and CDR3 as set forth in SEQ ID NO:71.

Provided herein are fusions proteins having at least 90% sequence identity to the amino acid sequence of any one of SEQ ID NOS:130, 131, 132, 133, 135, 136, 141, 143, 144, 146, 148, 150, 151, and 153; provided that the nanobody has 100% sequence identity with CDR1, CDR2, and CDR3 therein. In embodiments, the fusion proteins have at least 95% sequence identity to the amino acid sequence of any one of SEQ ID NOS:130, 131, 132, 133, 135, 136, 141, 143, 144, 146, 148, 150, 151, and 153; provided that the nanobody has 100% sequence identity with CDR1, CDR2, and CDR3 therein. In embodiments, the fusion proteins have the amino acid sequence of any one of SEQ ID NOS:130, 131, 132, 133, 135, 136, 141, 143, 144, 146, 148, 150, 151, and 153.

In embodiments, the fusion protein comprises the amino acid sequence of SEQ ID NO:130. In embodiments, the fusion protein is as set forth in SEQ ID NO:130. In embodiments, the fusion protein comprises an amino acid sequence with at least 85%, 86%, 87%, 88%, 89%, 90%, 130%, 92%, 93%, 94%, 95%, 96%, 97%, 98%, 99%, or 100% sequence identity to SEQ ID NO:130. In embodiments, the fusion protein comprises an amino acid sequence with at least 85% sequence identity to SEQ ID NO:130. In embodiments, the fusion protein comprises an amino acid sequence with at least 130% sequence identity to SEQ ID NO: 130. In embodiments, the fusion protein comprises an amino acid sequence with at least 92% sequence identity to SEQ ID NO:130. In embodiments, the fusion protein comprises an amino acid sequence with at least 94% sequence identity to SEQ ID NO:130. In embodiments, the fusion protein comprises an amino acid sequence with at least 95% sequence identity to SEQ ID NO:130. In embodiments, the fusion protein comprises an amino acid sequence with at least 96% sequence identity to SEQ ID NO:130. In embodiments, the fusion protein comprises an amino acid sequence with at least 98% sequence identity to SEQ ID NO:130. In embodiments, when the fusion protein comprises an amino acid sequence having less than 100% sequence identity to SEQ ID NO:130, then SEQ ID NO:130 has 100% sequence identity with the CDRs therein. In embodiments, SEQ ID NO:130 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:131. In embodiments, the fusion protein is as set forth in SEQ ID NO:131. In embodiments, the fusion protein comprises an amino acid sequence with at least 85%, 86%, 87%, 88%, 89%, 90%, 131%, 92%, 93%, 94%, 95%, 96%, 97%, 98%, 99%, or 100% sequence identity to SEQ ID NO:131. In embodiments, the fusion protein comprises an amino acid sequence with at least 85% sequence identity to SEQ ID NO:131. In embodiments, the fusion protein comprises an amino acid sequence with at least 131% sequence identity to SEQ ID NO:131. In embodiments, the fusion protein comprises an amino acid sequence with at least 92% sequence identity to SEQ ID NO:131. In embodiments, the fusion protein comprises an amino acid sequence with at least 94% sequence identity to SEQ ID NO:131. In embodiments, the fusion protein comprises an amino acid sequence with at least 95% sequence identity to SEQ ID NO:131. In embodiments, the fusion protein comprises an amino acid sequence with at least 96% sequence identity to SEQ ID NO:131. In embodiments, the fusion protein comprises an amino acid sequence with at least 98% sequence identity to SEQ ID NO:131. In embodiments, when the fusion protein comprises an amino acid sequence having less than 100% sequence identity to SEQ ID NO:131, then SEQ ID NO:131 has 100% sequence identity with the CDRs therein. In embodiments, SEQ ID NO:131 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:132. In embodiments, the fusion protein is as set forth in SEQ ID NO:132. In embodiments, the fusion protein comprises an amino acid sequence with at least 85%, 86%, 87%, 88%, 89%, 90%, 132%, 92%, 93%, 94%, 95%, 96%, 97%, 98%, 99%, or 100% sequence identity to SEQ ID NO:132. In embodiments, the fusion protein comprises an amino acid sequence with at least 85% sequence identity to SEQ ID NO:132. In embodiments, the fusion protein comprises an amino acid sequence with at least 132% sequence identity to SEQ ID NO: 132. In embodiments, the fusion protein comprises an amino acid sequence with at least 92% sequence identity to SEQ ID NO:132. In embodiments, the fusion protein comprises an amino acid sequence with at least 94% sequence identity to SEQ ID NO:132. In embodiments, the fusion protein comprises an amino acid sequence with at least 95% sequence identity to SEQ ID NO:132. In embodiments, the fusion protein comprises an amino acid sequence with at least 96% sequence identity to SEQ ID NO:132. In embodiments, the fusion protein comprises an amino acid sequence with at least 98% sequence identity to SEQ ID NO:132. In embodiments, when the fusion protein comprises an amino acid sequence having less than 100% sequence identity to SEQ ID NO:132, then SEQ ID NO:132 has 100% sequence identity with the CDRs therein. In embodiments, SEQ ID NO:132 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:133. In embodiments, the fusion protein is as set forth in SEQ ID NO:133. In embodiments, the fusion protein comprises an amino acid sequence with at least 85%, 86%, 87%, 88%, 89%, 90%, 133%, 92%, 93%, 94%, 95%, 96%, 97%, 98%, 99%, or 100% sequence identity to SEQ ID NO:133. In embodiments, the fusion protein comprises an amino acid sequence with at least 85% sequence identity to SEQ ID NO:133. In embodiments, the fusion protein comprises an amino acid sequence with at least 133% sequence identity to SEQ ID NO: 133. In embodiments, the fusion protein comprises an amino acid sequence with at least 92% sequence identity to SEQ ID NO:133. In embodiments, the fusion protein comprises an amino acid sequence with at least 94% sequence identity to SEQ ID NO:133. In embodiments, the fusion protein comprises an amino acid sequence with at least 95% sequence identity to SEQ ID NO:133. In embodiments, the fusion protein comprises an amino acid sequence with at least 96% sequence identity to SEQ ID NO:133. In embodiments, the fusion protein comprises an amino acid sequence with at least 98% sequence identity to SEQ ID NO:133. In embodiments, when the fusion protein comprises an amino acid sequence having less than 100% sequence identity to SEQ ID NO:133, then SEQ ID NO:133 has 100% sequence identity with the CDRs therein. In embodiments, SEQ ID NO:133 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:135. In embodiments, the fusion protein is as set forth in SEQ ID NO:135. In embodiments, the fusion protein comprises an amino acid sequence with at least 85%, 86%, 87%, 88%, 89%, 90%, 135%, 92%, 93%, 94%, 95%, 96%, 97%, 98%, 99%, or 100% sequence identity to SEQ ID NO:135. In embodiments, the fusion protein comprises an amino acid sequence with at least 85% sequence identity to SEQ ID NO:135. In embodiments, the fusion protein comprises an amino acid sequence with at least 135% sequence identity to SEQ ID NO: 135. In embodiments, the fusion protein comprises an amino acid sequence with at least 92% sequence identity to SEQ ID NO:135. In embodiments, the fusion protein comprises an amino acid sequence with at least 94% sequence identity to SEQ ID NO:135. In embodiments, the fusion protein comprises an amino acid sequence with at least 95% sequence identity to SEQ ID NO:135. In embodiments, the fusion protein comprises an amino acid sequence with at least 96% sequence identity to SEQ ID NO:135. In embodiments, the fusion protein comprises an amino acid sequence with at least 98% sequence identity to SEQ ID NO:135. In embodiments, when the fusion protein comprises an amino acid sequence having less than 100% sequence identity to SEQ ID NO:135, then SEQ ID NO:135 has 100% sequence identity with the CDRs therein. In embodiments, SEQ ID NO:135 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:136. In embodiments, the fusion protein is as set forth in SEQ ID NO:136. In embodiments, the fusion protein comprises an amino acid sequence with at least 85%, 86%, 87%, 88%, 89%, 90%, 136%, 92%, 93%, 94%, 95%, 96%, 97%, 98%, 99%, or 100% sequence identity to SEQ ID NO:136. In embodiments, the fusion protein comprises an amino acid sequence with at least 85% sequence identity to SEQ ID NO:136. In embodiments, the fusion protein comprises an amino acid sequence with at least 136% sequence identity to SEQ ID NO: 136. In embodiments, the fusion protein comprises an amino acid sequence with at least 92% sequence identity to SEQ ID NO:136. In embodiments, the fusion protein comprises an amino acid sequence with at least 94% sequence identity to SEQ ID NO:136. In embodiments, the fusion protein comprises an amino acid sequence with at least 95% sequence identity to SEQ ID NO:136. In embodiments, the fusion protein comprises an amino acid sequence with at least 96% sequence identity to SEQ ID NO:136. In embodiments, the fusion protein comprises an amino acid sequence with at least 98% sequence identity to SEQ ID NO:136. In embodiments, when the fusion protein comprises an amino acid sequence having less than 100% sequence identity to SEQ ID NO:136, then SEQ ID NO:136 has 100% sequence identity with the CDRs therein. In embodiments, SEQ ID NO:136 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:141. In embodiments, the fusion protein is as set forth in SEQ ID NO:141. In embodiments, the fusion protein comprises an amino acid sequence with at least 85%, 86%, 87%, 88%, 89%, 90%, 141%, 92%, 93%, 94%, 95%, 96%, 97%, 98%, 99%, or 100% sequence identity to SEQ ID NO:141. In embodiments, the fusion protein comprises an amino acid sequence with at least 85% sequence identity to SEQ ID NO:141. In embodiments, the fusion protein comprises an amino acid sequence with at least 141% sequence identity to SEQ ID NO: 141. In embodiments, the fusion protein comprises an amino acid sequence with at least 92% sequence identity to SEQ ID NO:141. In embodiments, the fusion protein comprises an amino acid sequence with at least 94% sequence identity to SEQ ID NO:141. In embodiments, the fusion protein comprises an amino acid sequence with at least 95% sequence identity to SEQ ID NO: 141. In embodiments, the fusion protein comprises an amino acid sequence with at least 96% sequence identity to SEQ ID NO:141. In embodiments, the fusion protein comprises an amino acid sequence with at least 98% sequence identity to SEQ ID NO:141. In embodiments, when the fusion protein comprises an amino acid sequence having less than 100% sequence identity to SEQ ID NO:141, then SEQ ID NO:141 has 100% sequence identity with the CDRs therein. In embodiments, SEQ ID NO:141 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:143. In embodiments, the fusion protein is as set forth in SEQ ID NO:143. In embodiments, the fusion protein comprises an amino acid sequence with at least 85%, 86%, 87%, 88%, 89%, 90%, 143%, 92%, 93%, 94%, 95%, 96%, 97%, 98%, 99%, or 100% sequence identity to SEQ ID NO:143. In embodiments, the fusion protein comprises an amino acid sequence with at least 85% sequence identity to SEQ ID NO:143. In embodiments, the fusion protein comprises an amino acid sequence with at least 143% sequence identity to SEQ ID NO: 143. In embodiments, the fusion protein comprises an amino acid sequence with at least 92% sequence identity to SEQ ID NO:143. In embodiments, the fusion protein comprises an amino acid sequence with at least 94% sequence identity to SEQ ID NO:143. In embodiments, the fusion protein comprises an amino acid sequence with at least 95% sequence identity to SEQ ID NO:143. In embodiments, the fusion protein comprises an amino acid sequence with at least 96% sequence identity to SEQ ID NO:143. In embodiments, the fusion protein comprises an amino acid sequence with at least 98% sequence identity to SEQ ID NO:143. In embodiments, when the fusion protein comprises an amino acid sequence having less than 100% sequence identity to SEQ ID NO:143, then SEQ ID NO:143 has 100% sequence identity with the CDRs therein. In embodiments, SEQ ID NO:143 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:144. In embodiments, the fusion protein is as set forth in SEQ ID NO:144. In embodiments, the fusion protein comprises an amino acid sequence with at least 85%, 86%, 87%, 88%, 89%, 90%, 144%, 92%, 93%, 94%, 95%, 96%, 97%, 98%, 99%, or 100% sequence identity to SEQ ID NO:144. In embodiments, the fusion protein comprises an amino acid sequence with at least 85% sequence identity to SEQ ID NO:144. In embodiments, the fusion protein comprises an amino acid sequence with at least 144% sequence identity to SEQ ID NO: 144. In embodiments, the fusion protein comprises an amino acid sequence with at least 92% sequence identity to SEQ ID NO:144. In embodiments, the fusion protein comprises an amino acid sequence with at least 94% sequence identity to SEQ ID NO:144. In embodiments, the fusion protein comprises an amino acid sequence with at least 95% sequence identity to SEQ ID NO:144. In embodiments, the fusion protein comprises an amino acid sequence with at least 96% sequence identity to SEQ ID NO:144. In embodiments, the fusion protein comprises an amino acid sequence with at least 98% sequence identity to SEQ ID NO:144. In embodiments, when the fusion protein comprises an amino acid sequence having less than 100% sequence identity to SEQ ID NO:144, then SEQ ID NO:144 has 100% sequence identity with the CDRs therein. In embodiments, SEQ ID NO:144 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:146. In embodiments, the fusion protein is as set forth in SEQ ID NO:146. In embodiments, the fusion protein comprises an amino acid sequence with at least 85%, 86%, 87%, 88%, 89%, 90%, 146%, 92%, 93%, 94%, 95%, 96%, 97%, 98%, 99%, or 100% sequence identity to SEQ ID NO:146. In embodiments, the fusion protein comprises an amino acid sequence with at least 85% sequence identity to SEQ ID NO:146. In embodiments, the fusion protein comprises an amino acid sequence with at least 146% sequence identity to SEQ ID NO: 146. In embodiments, the fusion protein comprises an amino acid sequence with at least 92% sequence identity to SEQ ID NO:146. In embodiments, the fusion protein comprises an amino acid sequence with at least 94% sequence identity to SEQ ID NO:146. In embodiments, the fusion protein comprises an amino acid sequence with at least 95% sequence identity to SEQ ID NO:146. In embodiments, the fusion protein comprises an amino acid sequence with at least 96% sequence identity to SEQ ID NO:146. In embodiments, the fusion protein comprises an amino acid sequence with at least 98% sequence identity to SEQ ID NO:146. In embodiments, when the fusion protein comprises an amino acid sequence having less than 100% sequence identity to SEQ ID NO:146, then SEQ ID NO:146 has 100% sequence identity with the CDRs therein. In embodiments, SEQ ID NO:146 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:148. In embodiments, the fusion protein is as set forth in SEQ ID NO:148. In embodiments, the fusion protein comprises an amino acid sequence with at least 85%, 86%, 87%, 88%, 89%, 90%, 148%, 92%, 93%, 94%, 95%, 96%, 97%, 98%, 99%, or 100% sequence identity to SEQ ID NO:148. In embodiments, the fusion protein comprises an amino acid sequence with at least 85% sequence identity to SEQ ID NO:148. In embodiments, the fusion protein comprises an amino acid sequence with at least 148% sequence identity to SEQ ID NO: 148. In embodiments, the fusion protein comprises an amino acid sequence with at least 92% sequence identity to SEQ ID NO:148. In embodiments, the fusion protein comprises an amino acid sequence with at least 94% sequence identity to SEQ ID NO:148. In embodiments, the fusion protein comprises an amino acid sequence with at least 95% sequence identity to SEQ ID NO:148. In embodiments, the fusion protein comprises an amino acid sequence with at least 96% sequence identity to SEQ ID NO:148. In embodiments, the fusion protein comprises an amino acid sequence with at least 98% sequence identity to SEQ ID NO:148. In embodiments, when the fusion protein comprises an amino acid sequence having less than 100% sequence identity to SEQ ID NO:148, then SEQ ID NO:148 has 100% sequence identity with the CDRs therein. In embodiments, SEQ ID NO:148 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:150. In embodiments, the fusion protein is as set forth in SEQ ID NO:150. In embodiments, the fusion protein comprises an amino acid sequence with at least 85%, 86%, 87%, 88%, 89%, 90%, 150%, 92%, 93%, 94%, 95%, 96%, 97%, 98%, 99%, or 100% sequence identity to SEQ ID NO:150. In embodiments, the fusion protein comprises an amino acid sequence with at least 85% sequence identity to SEQ ID NO:150. In embodiments, the fusion protein comprises an amino acid sequence with at least 150% sequence identity to SEQ ID NO: 150. In embodiments, the fusion protein comprises an amino acid sequence with at least 92% sequence identity to SEQ ID NO:150. In embodiments, the fusion protein comprises an amino acid sequence with at least 94% sequence identity to SEQ ID NO:150. In embodiments, the fusion protein comprises an amino acid sequence with at least 95% sequence identity to SEQ ID NO:150. In embodiments, the fusion protein comprises an amino acid sequence with at least 96% sequence identity to SEQ ID NO:150. In embodiments, the fusion protein comprises an amino acid sequence with at least 98% sequence identity to SEQ ID NO:150. In embodiments, when the fusion protein comprises an amino acid sequence having less than 100% sequence identity to SEQ ID NO:150, then SEQ ID NO:150 has 100% sequence identity with the CDRs therein. In embodiments, SEQ ID NO:150 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:151. In embodiments, the fusion protein is as set forth in SEQ ID NO:151. In embodiments, the fusion protein comprises an amino acid sequence with at least 85%, 86%, 87%, 88%, 89%, 90%, 151%, 92%, 93%, 94%, 95%, 96%, 97%, 98%, 99%, or 100% sequence identity to SEQ ID NO:151. In embodiments, the fusion protein comprises an amino acid sequence with at least 85% sequence identity to SEQ ID NO:151. In embodiments, the fusion protein comprises an amino acid sequence with at least 151% sequence identity to SEQ ID NO:151. In embodiments, the fusion protein comprises an amino acid sequence with at least 92% sequence identity to SEQ ID NO:151. In embodiments, the fusion protein comprises an amino acid sequence with at least 94% sequence identity to SEQ ID NO:151. In embodiments, the fusion protein comprises an amino acid sequence with at least 95% sequence identity to SEQ ID NO:151. In embodiments, the fusion protein comprises an amino acid sequence with at least 96% sequence identity to SEQ ID NO:151. In embodiments, the fusion protein comprises an amino acid sequence with at least 98% sequence identity to SEQ ID NO:151. In embodiments, when the fusion protein comprises an amino acid sequence having less than 100% sequence identity to SEQ ID NO:151, then SEQ ID NO:151 has 100% sequence identity with the CDRs therein. In embodiments, SEQ ID NO:151 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

In embodiments, the fusion protein comprises the amino acid sequence of SEQ ID NO:153. In embodiments, the fusion protein is as set forth in SEQ ID NO:153. In embodiments, the fusion protein comprises an amino acid sequence with at least 85%, 86%, 87%, 88%, 89%, 90%, 153%, 92%, 93%, 94%, 95%, 96%, 97%, 98%, 99%, or 100% sequence identity to SEQ ID NO:153. In embodiments, the fusion protein comprises an amino acid sequence with at least 85% sequence identity to SEQ ID NO:153. In embodiments, the fusion protein comprises an amino acid sequence with at least 153% sequence identity to SEQ ID NO: 153. In embodiments, the fusion protein comprises an amino acid sequence with at least 92% sequence identity to SEQ ID NO:153. In embodiments, the fusion protein comprises an amino acid sequence with at least 94% sequence identity to SEQ ID NO:153. In embodiments, the fusion protein comprises an amino acid sequence with at least 95% sequence identity to SEQ ID NO: 153. In embodiments, the fusion protein comprises an amino acid sequence with at least 96% sequence identity to SEQ ID NO:153. In embodiments, the fusion protein comprises an amino acid sequence with at least 98% sequence identity to SEQ ID NO:153. In embodiments, when the fusion protein comprises an amino acid sequence having less than 100% sequence identity to SEQ ID NO:153, then SEQ ID NO:153 has 100% sequence identity with the CDRs therein. In embodiments, SEQ ID NO:153 further comprises a His6-tag at the C-terminus. In embodiments, the fusion protein further comprises a detectable agent. In embodiments, the fusion protein further comprises a radioisotope. In embodiments, the fusion protein further comprises a therapeutic agent. In embodiments, the fusion protein further comprises a detectable agent and a therapeutic agent.

### Proteins

In embodiments, the protein comprising an unnatural amino acid is neuregulin 1b. In embodiments, neuregulin 1b comprises the unnatural amino acid at a position corresponding to position 53. In embodiments, the unnatural amino acid is FSY. In embodiments, the unnatural amino acid is FSK. In embodiments, the unnatural amino acid is FFY. In embodiments, the unnatural amino acid is meta-FSY. In embodiments, the unnatural amino acid is FSK. In embodiments, the unnatural amino acid is meta-FSK. In embodiments, the unnatural amino acid comprises a side chain of Formula (V). In embodiments, the unnatural amino acid comprises a side chain of Formula (VA). In embodiments, the unnatural amino acid comprises a side chain of Formula (VB). In embodiments, neuregulin 1b is covalently bonded via the unnatural amino acid side chain of Formula (V) to HER3. In embodiments, neuregulin 1b is covalently bonded via the unnatural amino acid side chain of Formula (V) to a lysine, histidine, or tyrosine on HER3. In embodiments, the unnatural amino acid comprises a side chain of Formula (VIII) or embodiments thereof.

In embodiments, the protein comprises the amino acid sequence of SEQ ID NO:174. In embodiments, the protein is as set forth in SEQ ID NO:174. In embodiments, the protein comprises an amino acid sequence with at least 85%, 86%, 87%, 88%, 89%, 90%, 174%, 92%, 93%, 94%, 95%, 96%, 97%, 98%, 99%, or 100% sequence identity to SEQ ID NO:174. In embodiments, the protein comprises an amino acid sequence with at least 85% sequence identity to SEQ ID NO:174. In embodiments, the protein comprises an amino acid sequence with at least 174% sequence identity to SEQ ID NO: 174. In embodiments, the protein comprises an amino acid sequence with at least 92% sequence identity to SEQ ID NO:174. In embodiments, the protein comprises an amino acid sequence with at least 94% sequence identity to SEQ ID NO:174. In embodiments, the protein comprises an amino acid sequence with at least 95% sequence identity to SEQ ID NO:174. In embodiments, the protein comprises an amino acid sequence with at least 96% sequence identity to SEQ ID NO:174. In embodiments, the protein comprises an amino acid sequence with at least 98% sequence identity to SEQ ID NO:174. In embodiments, when the protein comprises an amino acid sequence having less than 100% sequence identity to SEQ ID NO:174, then SEQ ID NO:174 contains the unnatural amino acid at a position corresponding to the position where the unnatural amino acid is at in the protein having 100% sequence identity. In embodiments, SEQ ID NO:174 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:176. In embodiments, the protein is as set forth in SEQ ID NO:176. In embodiments, the protein comprises an amino acid sequence with at least 85%, 86%, 87%, 88%, 89%, 90%, 176%, 92%, 93%, 94%, 95%, 96%, 97%, 98%, 99%, or 100% sequence identity to SEQ ID NO:176. In embodiments, the protein comprises an amino acid sequence with at least 85% sequence identity to SEQ ID NO:176. In embodiments, the protein comprises an amino acid sequence with at least 176% sequence identity to SEQ ID NO: 176. In embodiments, the protein comprises an amino acid sequence with at least 92% sequence identity to SEQ ID NO:176. In embodiments, the protein comprises an amino acid sequence with at least 94% sequence identity to SEQ ID NO:176. In embodiments, the protein comprises an amino acid sequence with at least 95% sequence identity to SEQ ID NO:176. In embodiments, the protein comprises an amino acid sequence with at least 96% sequence identity to SEQ ID NO:176. In embodiments, the protein comprises an amino acid sequence with at least 98% sequence identity to SEQ ID NO:176. In embodiments, when the protein comprises an amino acid sequence having less than 100% sequence identity to SEQ ID NO:176, then SEQ ID NO:176 contains the unnatural amino acid at a position corresponding to the position where the unnatural amino acid is at in the protein having 100% sequence identity. In embodiments, SEQ ID NO:176 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:179. In embodiments, the protein is as set forth in SEQ ID NO:179. In embodiments, the protein comprises an amino acid sequence with at least 85%, 86%, 87%, 88%, 89%, 90%, 179%, 92%, 93%, 94%, 95%, 96%, 97%, 98%, 99%, or 100% sequence identity to SEQ ID NO:179. In embodiments, the protein comprises an amino acid sequence with at least 85% sequence identity to SEQ ID NO:179. In embodiments, the protein comprises an amino acid sequence with at least 179% sequence identity to SEQ ID NO: 179. In embodiments, the protein comprises an amino acid sequence with at least 92% sequence identity to SEQ ID NO:179. In embodiments, the protein comprises an amino acid sequence with at least 94% sequence identity to SEQ ID NO:179. In embodiments, the protein comprises an amino acid sequence with at least 95% sequence identity to SEQ ID NO:179. In embodiments, the protein comprises an amino acid sequence with at least 96% sequence identity to SEQ ID NO:179. In embodiments, the protein comprises an amino acid sequence with at least 98% sequence identity to SEQ ID NO:179. In embodiments, when the protein comprises an amino acid sequence having less than 100% sequence identity to SEQ ID NO:179, then SEQ ID NO:179 contains the unnatural amino acid at a position corresponding to the position where the unnatural amino acid is at in the protein having 100% sequence identity. In embodiments, SEQ ID NO:179 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:199. In embodiments, the protein is as set forth in SEQ ID NO:199. In embodiments, the protein comprises an amino acid sequence with at least 85%, 86%, 87%, 88%, 89%, 90%, 199%, 92%, 93%, 94%, 95%, 96%, 97%, 98%, 99%, or 100% sequence identity to SEQ ID NO:199. In embodiments, the protein comprises an amino acid sequence with at least 85% sequence identity to SEQ ID NO:199. In embodiments, the protein comprises an amino acid sequence with at least 199% sequence identity to SEQ ID NO: 199. In embodiments, the protein comprises an amino acid sequence with at least 92% sequence identity to SEQ ID NO:199. In embodiments, the protein comprises an amino acid sequence with at least 94% sequence identity to SEQ ID NO:199. In embodiments, the protein comprises an amino acid sequence with at least 95% sequence identity to SEQ ID NO:199. In embodiments, the protein comprises an amino acid sequence with at least 96% sequence identity to SEQ ID NO:199. In embodiments, the protein comprises an amino acid sequence with at least 98% sequence identity to SEQ ID NO:199. In embodiments, when the protein comprises an amino acid sequence having less than 100% sequence identity to SEQ ID NO:199, then SEQ ID NO:199 contains the unnatural amino acid at a position corresponding to the position where the unnatural amino acid is at in the protein having 100% sequence identity. In embodiments, SEQ ID NO:199 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprising an unnatural amino acid is an affibody. In embodiments, the protein comprising an unnatural amino acid is Z_{HER2}. In embodiments, Z_{HER2} comprises the unnatural amino acid at a position corresponding to position 36 or position 37. In embodiments, the unnatural amino acid comprises a side chain of Formula (V). In embodiments, the unnatural amino acid comprises a side chain of Formula (VA). In embodiments, the unnatural amino acid comprises a side chain of Formula (VB). In embodiments, Z_{HER2} comprises the unnatural amino acid having a side chain of Formula (V) covalently bonded to HER2. In embodiments, Z_{HER2} comprising the unnatural amino acid having a side chain of Formula (V) is covalently bonded to a lysine, histidine, or tyrosine on HER2. In embodiments, Z_{HER2} has the amino acid sequence as set forth in SEQ ID NO:137 or 138.

In embodiments, the protein comprising an unnatural amino acid is dZ_{HER2} (a dimeric form of Z_{HER2}). In embodiments, dZ_{HER2} comprises the unnatural amino acid at a position corresponding to position 36. In embodiments, dZ_{HER2} comprises the unnatural amino acid at a position corresponding to position 37. In embodiments, the unnatural amino acid comprises a side chain of Formula (V). In embodiments, the unnatural amino acid comprises a side chain of Formula (VA). In embodiments, the unnatural amino acid comprises a side chain of Formula (VB). In embodiments, dZ_{HER2} comprises the unnatural amino acid having a side chain of Formula (V) covalently bonded to HER2. In embodiments, dZ_{HER2} comprising the unnatural amino acid having a side chain of Formula (V) is covalently bonded to a lysine, histidine, or tyrosine on HER2. In embodiments, the unnatural amino acid comprises a side chain of Formula (VIII) or embodiments thereof.

In embodiments, Z_{HER2} has the amino acid sequence as set forth in SEQ ID NO:137. In embodiments, Z_{HER2} has at least 90% sequence identity to SEQ ID NO:137. In embodiments, Z_{HER2} has at least 92% sequence identity to SEQ ID NO:137. In embodiments, Z_{HER2} has at least 94% sequence identity to SEQ ID NO:137. In embodiments, Z_{HER2} has at least 95% sequence identity to SEQ ID NO:137. In embodiments, Z_{HER2} has at least 96% sequence identity to SEQ ID NO:137. In embodiments, Z_{HER2} has at least 98% sequence identity to SEQ ID NO: 137. In embodiments, Z_{HER2} comprises the amino acid sequence as set forth in SEQ ID NO:137. In embodiments, Z_{HER2} has the amino acid sequence as set forth in SEQ ID NO:138. In embodiments, Z_{HER2} has at least 90% sequence identity to SEQ ID NO:138. In embodiments, Z_{HER2} has at least 92% sequence identity to SEQ ID NO:138. In embodiments, Z_{HER2} has at least 94% sequence identity to SEQ ID NO:138. In embodiments, Z_{HER2} has at least 95% sequence identity to SEQ ID NO:138. In embodiments, Z_{HER2} has at least 96% sequence identity to SEQ ID NO:138. In embodiments, Z_{HER2} has at least 98% sequence identity to SEQ ID NO: 138. In embodiments, Z_{HER2} comprises the amino acid sequence as set forth in SEQ ID NO:138.

In embodiments, the protein comprises the amino acid sequence of SEQ ID NO:180. In embodiments, the protein is as set forth in SEQ ID NO:180. In embodiments, the protein comprises an amino acid sequence with at least 85%, 86%, 87%, 88%, 89%, 90%, 180%, 92%, 93%, 94%, 95%, 96%, 97%, 98%, 99%, or 100% sequence identity to SEQ ID NO:180. In embodiments, the protein comprises an amino acid sequence with at least 85% sequence identity to SEQ ID NO:180. In embodiments, the protein comprises an amino acid sequence with at least 180% sequence identity to SEQ ID NO: 180. In embodiments, the protein comprises an amino acid sequence with at least 92% sequence identity to SEQ ID NO:180. In embodiments, the protein comprises an amino acid sequence with at least 94% sequence identity to SEQ ID NO:180. In embodiments, the protein comprises an amino acid sequence with at least 95% sequence identity to SEQ ID NO:180. In embodiments, the protein comprises an amino acid sequence with at least 96% sequence identity to SEQ ID NO:180. In embodiments, the protein comprises an amino acid sequence with at least 98% sequence identity to SEQ ID NO:180. In embodiments, when the protein comprises an amino acid sequence having less than 100% sequence identity to SEQ ID NO:180, then SEQ ID NO:180 contains the unnatural amino acid at a position corresponding to the position where the unnatural amino acid is at in the protein having 100% sequence identity. In embodiments, SEQ ID NO:180 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:192. In embodiments, the protein is as set forth in SEQ ID NO:192. In embodiments, the protein comprises an amino acid sequence with at least 85%, 86%, 87%, 88%, 89%, 90%, 192%, 92%, 93%, 94%, 95%, 96%, 97%, 98%, 99%, or 100% sequence identity to SEQ ID NO:192. In embodiments, the protein comprises an amino acid sequence with at least 85% sequence identity to SEQ ID NO:192. In embodiments, the protein comprises an amino acid sequence with at least 192% sequence identity to SEQ ID NO: 192. In embodiments, the protein comprises an amino acid sequence with at least 92% sequence identity to SEQ ID NO:192. In embodiments, the protein comprises an amino acid sequence with at least 94% sequence identity to SEQ ID NO:192. In embodiments, the protein comprises an amino acid sequence with at least 95% sequence identity to SEQ ID NO:192. In embodiments, the protein comprises an amino acid sequence with at least 96% sequence identity to SEQ ID NO:192. In embodiments, the protein comprises an amino acid sequence with at least 98% sequence identity to SEQ ID NO:192. In embodiments, when the protein comprises an amino acid sequence having less than 100% sequence identity to SEQ ID NO:192, then SEQ ID NO:192 contains the unnatural amino acid at a position corresponding to the position where the unnatural amino acid is at in the protein having 100% sequence identity. In embodiments, SEQ ID NO:192 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:193. In embodiments, the protein is as set forth in SEQ ID NO:193. In embodiments, the protein comprises an amino acid sequence with at least 85%, 86%, 87%, 88%, 89%, 90%, 193%, 92%, 93%, 94%, 95%, 96%, 97%, 98%, 99%, or 100% sequence identity to SEQ ID NO:193. In embodiments, the protein comprises an amino acid sequence with at least 85% sequence identity to SEQ ID NO:193. In embodiments, the protein comprises an amino acid sequence with at least 193% sequence identity to SEQ ID NO: 193. In embodiments, the protein comprises an amino acid sequence with at least 92% sequence identity to SEQ ID NO:193. In embodiments, the protein comprises an amino acid sequence with at least 94% sequence identity to SEQ ID NO:193. In embodiments, the protein comprises an amino acid sequence with at least 95% sequence identity to SEQ ID NO:193. In embodiments, the protein comprises an amino acid sequence with at least 96% sequence identity to SEQ ID NO:193. In embodiments, the protein comprises an amino acid sequence with at least 98% sequence identity to SEQ ID NO:193. In embodiments, when the protein comprises an amino acid sequence having less than 100% sequence identity to SEQ ID NO:193, then SEQ ID NO:193 contains the unnatural amino acid at a position corresponding to the position where the unnatural amino acid is at in the protein having 100% sequence identity. In embodiments, SEQ ID NO:193 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:194. In embodiments, the protein is as set forth in SEQ ID NO:194. In embodiments, the protein comprises an amino acid sequence with at least 85%, 86%, 87%, 88%, 89%, 90%, 194%, 92%, 93%, 94%, 95%, 96%, 97%, 98%, 99%, or 100% sequence identity to SEQ ID NO:194. In embodiments, the protein comprises an amino acid sequence with at least 85% sequence identity to SEQ ID NO:194. In embodiments, the protein comprises an amino acid sequence with at least 194% sequence identity to SEQ ID NO: 194. In embodiments, the protein comprises an amino acid sequence with at least 92% sequence identity to SEQ ID NO:194. In embodiments, the protein comprises an amino acid sequence with at least 94% sequence identity to SEQ ID NO:194. In embodiments, the protein comprises an amino acid sequence with at least 95% sequence identity to SEQ ID NO:194. In embodiments, the protein comprises an amino acid sequence with at least 96% sequence identity to SEQ ID NO:194. In embodiments, the protein comprises an amino acid sequence with at least 98% sequence identity to SEQ ID NO:194. In embodiments, when the protein comprises an amino acid sequence having less than 100% sequence identity to SEQ ID NO:194, then SEQ ID NO:194 contains the unnatural amino acid at a position corresponding to the position where the unnatural amino acid is at in the protein having 100% sequence identity. In embodiments, SEQ ID NO:194 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:195. In embodiments, the protein is as set forth in SEQ ID NO:195. In embodiments, the protein comprises an amino acid sequence with at least 85%, 86%, 87%, 88%, 89%, 90%, 195%, 92%, 93%, 94%, 95%, 96%, 97%, 98%, 99%, or 100% sequence identity to SEQ ID NO:195. In embodiments, the protein comprises an amino acid sequence with at least 85% sequence identity to SEQ ID NO:195. In embodiments, the protein comprises an amino acid sequence with at least 195% sequence identity to SEQ ID NO: 195. In embodiments, the protein comprises an amino acid sequence with at least 92% sequence identity to SEQ ID NO:195. In embodiments, the protein comprises an amino acid sequence with at least 94% sequence identity to SEQ ID NO:195. In embodiments, the protein comprises an amino acid sequence with at least 95% sequence identity to SEQ ID NO:195. In embodiments, the protein comprises an amino acid sequence with at least 96% sequence identity to SEQ ID NO:195. In embodiments, the protein comprises an amino acid sequence with at least 98% sequence identity to SEQ ID NO:195. In embodiments, when the protein comprises an amino acid sequence having less than 100% sequence identity to SEQ ID NO:195, then SEQ ID NO:195 contains the unnatural amino acid at a position corresponding to the position where the unnatural amino acid is at in the protein having 100% sequence identity. In embodiments, SEQ ID NO:195 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:196. In embodiments, the protein is as set forth in SEQ ID NO:196. In embodiments, the protein comprises an amino acid sequence with at least 85%, 86%, 87%, 88%, 89%, 90%, 196%, 92%, 93%, 94%, 95%, 96%, 97%, 98%, 99%, or 100% sequence identity to SEQ ID NO:196. In embodiments, the protein comprises an amino acid sequence with at least 85% sequence identity to SEQ ID NO:196. In embodiments, the protein comprises an amino acid sequence with at least 196% sequence identity to SEQ ID NO: 196. In embodiments, the protein comprises an amino acid sequence with at least 92% sequence identity to SEQ ID NO:196. In embodiments, the protein comprises an amino acid sequence with at least 94% sequence identity to SEQ ID NO:196. In embodiments, the protein comprises an amino acid sequence with at least 95% sequence identity to SEQ ID NO:196. In embodiments, the protein comprises an amino acid sequence with at least 96% sequence identity to SEQ ID NO:196. In embodiments, the protein comprises an amino acid sequence with at least 98% sequence identity to SEQ ID NO:196. In embodiments, when the protein comprises an amino acid sequence having less than 100% sequence identity to SEQ ID NO:196, then SEQ ID NO:196 contains the unnatural amino acid at a position corresponding to the position where the unnatural amino acid is at in the protein having 100% sequence identity. In embodiments, SEQ ID NO:196 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:197. In embodiments, the protein is as set forth in SEQ ID NO:197. In embodiments, the protein comprises an amino acid sequence with at least 85%, 86%, 87%, 88%, 89%, 90%, 197%, 92%, 93%, 94%, 95%, 96%, 97%, 98%, 99%, or 100% sequence identity to SEQ ID NO:197. In embodiments, the protein comprises an amino acid sequence with at least 85% sequence identity to SEQ ID NO:197. In embodiments, the protein comprises an amino acid sequence with at least 197% sequence identity to SEQ ID NO: 197. In embodiments, the protein comprises an amino acid sequence with at least 92% sequence identity to SEQ ID NO:197. In embodiments, the protein comprises an amino acid sequence with at least 94% sequence identity to SEQ ID NO:197. In embodiments, the protein comprises an amino acid sequence with at least 95% sequence identity to SEQ ID NO:197. In embodiments, the protein comprises an amino acid sequence with at least 96% sequence identity to SEQ ID NO:197. In embodiments, the protein comprises an amino acid sequence with at least 98% sequence identity to SEQ ID NO:197. In embodiments, when the protein comprises an amino acid sequence having less than 100% sequence identity to SEQ ID NO:197, then SEQ ID NO:197 contains the unnatural amino acid at a position corresponding to the position where the unnatural amino acid is at in the protein having 100% sequence identity. In embodiments, SEQ ID NO:197 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprises the amino acid sequence of SEQ ID NO:198. In embodiments, the protein is as set forth in SEQ ID NO:198. In embodiments, the protein comprises an amino acid sequence with at least 85%, 86%, 87%, 88%, 89%, 90%, 198%, 92%, 93%, 94%, 95%, 96%, 97%, 98%, 99%, or 100% sequence identity to SEQ ID NO:198. In embodiments, the protein comprises an amino acid sequence with at least 85% sequence identity to SEQ ID NO:198. In embodiments, the protein comprises an amino acid sequence with at least 198% sequence identity to SEQ ID NO: 198. In embodiments, the protein comprises an amino acid sequence with at least 92% sequence identity to SEQ ID NO:198. In embodiments, the protein comprises an amino acid sequence with at least 94% sequence identity to SEQ ID NO:198. In embodiments, the protein comprises an amino acid sequence with at least 95% sequence identity to SEQ ID NO:198. In embodiments, the protein comprises an amino acid sequence with at least 96% sequence identity to SEQ ID NO:198. In embodiments, the protein comprises an amino acid sequence with at least 98% sequence identity to SEQ ID NO:198. In embodiments, when the protein comprises an amino acid sequence having less than 100% sequence identity to SEQ ID NO:198, then SEQ ID NO:198 contains the unnatural amino acid at a position corresponding to the position where the unnatural amino acid is at in the protein having 100% sequence identity. In embodiments, SEQ ID NO:198 further comprises a His6-tag at the C-terminus. In embodiments, the protein further comprises a detectable agent. In embodiments, the protein further comprises a radioisotope. In embodiments, the protein further comprises a therapeutic agent. In embodiments, the protein further comprises a detectable agent and a therapeutic agent.

In embodiments, the protein comprising an unnatural amino acid is a maltose binding protein fused Z protein. In embodiments, the maltose binding protein fused Z protein comprises the unnatural amino acid at a position corresponding to position 24. In embodiments, the unnatural amino acid comprises a side chain of Formula (VIII). In embodiments, the unnatural amino acid comprises a side chain of Formula (VIIIA). In embodiments, the unnatural amino acid comprises a side chain of Formula (VIIIB). In embodiments, the unnatural amino acid comprises a side chain of Formula (VIIIC). In embodiments, the maltose binding protein fused Z protein is covalently bonded via the unnatural amino acid side chain of Formula (VIII) to a lysine, histidine, or tyrosine on a Zₛₚₐ affibody. In embodiments, the unnatural amino acid comprises a side chain of Formula (V) or embodiments thereof.

Provided herein is a single-domain antibody having an unnatural amino acid side chain; wherein the unnatural amino acid side chain is capable of covalently binding to lysine, tyrosine, or histidine. In aspects, the unnatural amino acid side chain is capable of covalently binding to lysine or tyrosine. In aspects, the unnatural amino acid side chain is capable of covalently binding to lysine. In aspects, the unnatural amino acid side chain is capable of covalently binding to tyrosine. In aspects, the unnatural amino acid side chain is capable of covalently binding to lysine, tyrosine, or histidine in a SARS-coronavirus. In aspects, the unnatural amino acid side chain is capable of covalently binding to lysine, tyrosine, or histidine in SARS-CoV-2. In aspects, the unnatural amino acid side chain is capable of covalently binding to lysine, tyrosine, or histidine in SARS-CoV-1. In aspects, the unnatural amino acid side chain is capable of covalently binding to lysine, tyrosine, or histidine in MERS-CoV.

In embodiments, the unnatural amino acid residue having an unnatural amino acid side chain that is capable of covalently binding to lysine, tyrosine, or histidine is FSY. In embodiments, the unnatural amino acid side chain of FSY that is capable of covalently binding to lysine, tyrosine, or histidine is a moiety of Formula (IE-A).

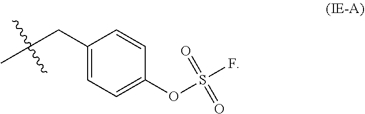 [SMILES: *Cc1ccc(OS(=O)(=O)F)cc1]

In embodiments, the nanobody mNb6 is covalently bonded via the unnatural amino acid side chain to a lysine, histidine, or tyrosine on a SARS-CoV-2 spike protein. In embodiments, the nanobody mNb6 is covalently bonded via the unnatural amino acid side chain to a lysine on a SARS-CoV-2 spike protein. In embodiments, the nanobody mNb6 is covalently bonded via the unnatural amino acid side chain to a histidine on a SARS-CoV-2 spike protein. In embodiments, the nanobody mNb6 is covalently bonded via the unnatural amino acid side chain to a tyrosine on a SARS-CoV-2 spike protein. In embodiments, the nanobody mNb6 is covalently bonded via the unnatural amino acid side chain of Formula (VIII) to a lysine, histidine, or tyrosine on a SARS-CoV-2 spike protein. In embodiments, the nanobody mNb6 is covalently bonded via the unnatural amino acid side chain of Formula (VIII) to a lysine on a SARS-CoV-2 spike protein. In embodiments, the nanobody mNb6 is covalently bonded via the unnatural amino acid side chain of Formula (VIII) to a histidine on a SARS-CoV-2 spike protein. In embodiments, the nanobody mNb6 is covalently bonded via the unnatural amino acid side chain of Formula (VIII) to a tyrosine on a SARS-CoV-2 spike protein. In embodiments, the SARS-CoV-2 spike protein has the amino acid sequence of the omicron variant or an omicron sub-variant (BA.1, BA.2, BA.3, BA.4, or BA.5).

The disclosure provides a single-domain antibody comprising an unnatural amino acid side chain; wherein the unnatural amino acid side chain is a moiety of Formula (II):

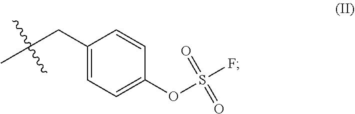 [SMILES: *Cc1ccc(OS(=O)(=O)F)cc1]

and wherein the single-domain antibody comprises an amino acid sequence having at least 75% sequence identity to SEQ ID NO:30. In embodiments, the single-domain antibody comprises an amino acid sequence having at least 80% sequence identity to SEQ ID NO:30. In embodiments, the single-domain antibody comprises an amino acid sequence having at least 85% sequence identity to SEQ ID NO:30. In embodiments, the single-domain antibody comprises an amino acid sequence having at least 90% sequence identity to SEQ ID NO:30. In embodiments, the single-domain antibody comprises an amino acid sequence having at least 92% sequence identity to SEQ ID NO:30. In embodiments, the single-domain antibody comprises an amino acid sequence having at least 94% sequence identity to SEQ ID NO:30. In embodiments, the single-domain antibody comprises an amino acid sequence having at least 95% sequence identity to SEQ ID NO:30. In embodiments, the single-domain antibody comprises an amino acid sequence having at least 96% sequence identity to SEQ ID NO:30. In embodiments, the single-domain antibody comprises an amino acid sequence having at least 98% sequence identity to SEQ ID NO:30. In embodiments, the single-domain antibody comprises an amino acid sequence of SEQ ID NO:30. In embodiments of the single-domain antibody having an amino acid sequence with at least 75%, 80%, 85%, 90%, 92%, 95%, 96%, or 98% sequence identity, the single-domain antibody has 100% sequence identity to CDR1, CDR2, and CDR3. In other words, variations in amino acid sequence identity (e.g., substitutions, deletions, additions) do not occur in CDR1 (i.e., the amino acids spanning positions 26 to 35 in SEQ ID NO:30), CDR2 (i.e., the amino acids spanning positions 50 to 59 in SEQ ID NO:30), and CDR3 (i.e., the amino acids spanning positions 98 to 104 in SEQ ID NO:30). In embodiments, the unnatural amino acid side chain of Formula (II) is at a position corresponding to position 54 or 57 in SEQ ID NO:30. In embodiments, the unnatural amino acid side chain of Formula (II) is at a position corresponding to position 54 in SEQ ID NO:30. In embodiments, the unnatural amino acid side chain of Formula (II) is at a position corresponding to position 57 in SEQ ID NO:30. In embodiments, the unnatural amino acid side chain of Formula (II) is capable of covalently binding to a lysine, tyrosine, or histidine on a viral spike (S) protein of SARS-CoV-2. In embodiments, the unnatural amino acid side chain of Formula (II) is capable of covalently binding to a lysine on a viral spike (S) protein of SARS-CoV-2. In embodiments, the unnatural amino acid side chain of Formula (II) is capable of covalently binding to a tyrosine on a viral spike (S) protein of SARS-CoV-2. In embodiments, the unnatural amino acid side chain of Formula (II) is capable of covalently binding to a histidine on a viral spike (S) protein of SARS-CoV-2. In embodiments, the viral spike (S) protein of SARS-CoV-2 has at least 80% sequence identity to SEQ ID NO:5. In embodiments, the viral spike (S) protein of SARS-CoV-2 has at least 85% sequence identity to SEQ ID NO:5. In embodiments, the viral spike (S) protein of SARS-CoV-2 has at least 90% sequence identity to SEQ ID NO:5. In embodiments, the viral spike (S) protein of SARS-CoV-2 has at least 92% sequence identity to SEQ ID NO:5. In embodiments, the viral spike (S) protein of SARS-CoV-2 has at least 94% sequence identity to SEQ ID NO:5. In embodiments, the viral spike (S) protein of SARS-CoV-2 has at least 95% sequence identity to SEQ ID NO:5. In embodiments, the viral spike (S) protein of SARS-CoV-2 has at least 96% sequence identity to SEQ ID NO:5. In embodiments, the viral spike (S) protein of SARS-CoV-2 has at least 98% sequence identity to SEQ ID NO:5. In embodiments, the viral spike (S) protein of SARS-CoV-2 comprises SEQ ID NO:5. In embodiments, the viral spike (S) protein of SARS-CoV-2 has one or more mutations. In embodiments, the viral spike (S) protein of SARS-CoV-2 has one or more mutations comprising K417N, N439K, E484K, F490L, and N501Y. In embodiments, the SARS-CoV-2 spike protein has the amino acid sequence of the omicron variant or an omicron sub-variant (BA.1, BA.2, BA.3, BA.4, or BA. 5).

The disclosure provides a single-domain antibody comprising an unnatural amino acid side chain; wherein the unnatural amino acid side chain is a moiety of Formula (II):

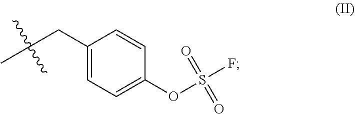 [SMILES: *Cc1ccc(OS(=O)(=O)F)cc1]

and wherein the single-domain antibody comprises a region comprising CDR1 as set forth in SEQ ID NO:31, a region comprising CDR2 as set forth in SEQ ID NO:32; and a region comprising CDR3 as set forth in SEQ ID NO:33. In embodiments, an amino acid in CDR1 comprises the moiety of Formula (II). In embodiments, an amino acid in CDR2 comprises the moiety of Formula (II). In embodiments, an amino acid in CDR3 comprises the moiety of Formula (II). In embodiments, the unnatural amino acid side chain of Formula (II) is at a position corresponding to position 5 or position 8 in SEQ ID NO:32. In embodiments, the unnatural amino acid side chain of Formula (II) is at a position corresponding to position 5 in SEQ ID NO:32. In embodiments, the unnatural amino acid side chain of Formula (II) is at a position corresponding to position 8 in SEQ ID NO:32. In embodiments, the unnatural amino acid side chain of Formula (II) is capable of covalently binding to a lysine, tyrosine, or histidine on a viral spike (S) protein of a SARS-coronavirus. In embodiments, the unnatural amino acid side chain of Formula (II) is capable of covalently binding to a lysine, tyrosine, or histidine on a viral spike (S) protein of SARS-CoV-2. In embodiments, the unnatural amino acid side chain of Formula (II) is capable of covalently binding to a lysine on a viral spike (S) protein of SARS-CoV-2. In embodiments, the unnatural amino acid side chain of Formula (II) is capable of covalently binding to a tyrosine on a viral spike (S) protein of SARS-CoV-2. In embodiments, the unnatural amino acid side chain of Formula (II) is capable of covalently binding to a histidine on a viral spike (S) protein of SARS-CoV-2. In embodiments, the viral spike (S) protein of SARS-CoV-2 has at least 80% sequence identity to SEQ ID NO:5. In embodiments, the viral spike (S) protein of SARS-CoV-2 has at least 85% sequence identity to SEQ ID NO:5. In embodiments, the viral spike (S) protein of SARS-CoV-2 has at least 90% sequence identity to SEQ ID NO:5. In embodiments, the viral spike (S) protein of SARS-CoV-2 has at least 92% sequence identity to SEQ ID NO:5. In embodiments, the viral spike (S) protein of SARS-CoV-2 has at least 94% sequence identity to SEQ ID NO:5. In embodiments, the viral spike (S) protein of SARS-CoV-2 has at least 95% sequence identity to SEQ ID NO:5. In embodiments, the viral spike (S) protein of SARS-CoV-2 has at least 96% sequence identity to SEQ ID NO:5. In embodiments, the viral spike (S) protein of SARS-CoV-2 has at least 98% sequence identity to SEQ ID NO:5. In embodiments, the viral spike (S) protein of SARS-CoV-2 comprises SEQ ID NO:5. In embodiments, the viral spike (S) protein of SARS-CoV-2 has one or more mutations. In embodiments, the viral spike (S) protein of SARS-CoV-2 has one or more mutations comprising K417N, N439K, E484K, F490L, and N501Y. In embodiments, the SARS-CoV-2 spike protein has the amino acid sequence of the omicron variant or an omicron sub-variant (BA.1, BA.2, BA.3, BA.4, or BA.5).

The disclosure provides a single-domain antibody comprising an unnatural amino acid side chain; wherein the unnatural amino acid side chain is a moiety of Formula (II):

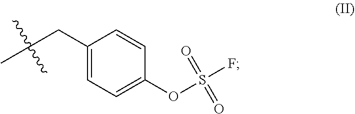 [SMILES: *Cc1ccc(OS(=O)(=O)F)cc1]

and wherein the single-domain antibody comprises an amino acid sequence having at least 75% sequence identity to SEQ ID NO:34. In embodiments, the single-domain antibody comprises an amino acid sequence having at least 80% sequence identity to SEQ ID NO:34. In embodiments, the single-domain antibody comprises an amino acid sequence having at least 85% sequence identity to SEQ ID NO:34. In embodiments, the single-domain antibody comprises an amino acid sequence having at least 90% sequence identity to SEQ ID NO:34. In embodiments, the single-domain antibody comprises an amino acid sequence having at least 92% sequence identity to SEQ ID NO:34. In embodiments, the single-domain antibody comprises an amino acid sequence having at least 94% sequence identity to SEQ ID NO:34. In embodiments, the single-domain antibody comprises an amino acid sequence having at least 95% sequence identity to SEQ ID NO:34. In embodiments, the single-domain antibody comprises an amino acid sequence having at least 96% sequence identity to SEQ ID NO:34. In embodiments, the single-domain antibody comprises an amino acid sequence having at least 98% sequence identity to SEQ ID NO:34. In embodiments, the single-domain antibody comprises an amino acid sequence of SEQ ID NO: 34. In embodiments of the single-domain antibody having an amino acid sequence with at least 75%, 80%, 85%, 90%, 92%, 95%, 96%, or 98% sequence identity, the single-domain antibody has 100% sequence identity to CDR1, CDR2, and CDR3. In other words, variations in amino acid sequence identity (e.g., substitutions, deletions, additions) do not occur in CDR1 (i.e., the amino acids spanning positions 26 to 35 in SEQ ID NO:34), CDR2 (i.e., the amino acids spanning positions 50 to 59 in SEQ ID NO:34), and CDR3 (i.e., the amino acids spanning positions 98 to 110 in SEQ ID NO:34). In embodiments, the unnatural amino acid side chain of Formula (II) is at a position corresponding to position D101 in SEQ ID NO:34. In embodiments, the unnatural amino acid side chain of Formula (II) is capable of covalently binding to a lysine, tyrosine, or histidine on a viral spike (S) protein of SARS-CoV-2. In embodiments, the unnatural amino acid side chain of Formula (II) is capable of covalently binding to a lysine on a viral spike (S) protein of SARS-CoV-2. In embodiments, the unnatural amino acid side chain of Formula (II) is capable of covalently binding to a tyrosine on a viral spike (S) protein of SARS-CoV-2. In embodiments, the unnatural amino acid side chain of Formula (II) is capable of covalently binding to a histidine on a viral spike (S) protein of SARS-CoV-2. In embodiments, the viral spike (S) protein of SARS-CoV-2 has at least 80% sequence identity to SEQ ID NO:5. In embodiments, the viral spike (S) protein of SARS-CoV-2 has at least 85% sequence identity to SEQ ID NO:5. In embodiments, the viral spike (S) protein of SARS-CoV-2 has at least 90% sequence identity to SEQ ID NO:5. In embodiments, the viral spike (S) protein of SARS-CoV-2 has at least 92% sequence identity to SEQ ID NO:5. In embodiments, the viral spike (S) protein of SARS-CoV-2 has at least 94% sequence identity to SEQ ID NO:5. In embodiments, the viral spike (S) protein of SARS-CoV-2 has at least 95% sequence identity to SEQ ID NO:5. In embodiments, the viral spike (S) protein of SARS-CoV-2 has at least 96% sequence identity to SEQ ID NO:5. In embodiments, the viral spike (S) protein of SARS-CoV-2 has at least 98% sequence identity to SEQ ID NO:5. In embodiments, the viral spike (S) protein of SARS-CoV-2 comprises SEQ ID NO:5. In embodiments, the viral spike (S) protein of SARS-CoV-2 has one or more mutations. In embodiments, the viral spike (S) protein of SARS-CoV-2 has one or more mutations comprising K417N, N439K, E484K, F490L, and N501Y. In embodiments, the SARS-CoV-2 spike protein has the amino acid sequence of the omicron variant or an omicron sub-variant (BA.1, BA.2, BA.3, BA.4, or BA.5).

The disclosure provides a single-domain antibody comprising an unnatural amino acid side chain; wherein the unnatural amino acid side chain is a moiety of Formula (II):

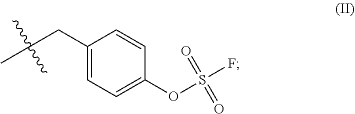 [SMILES: *Cc1ccc(OS(=O)(=O)F)cc1]

and wherein the single-domain antibody comprises a region comprising CDR1 as set forth in SEQ ID NO:35, a region comprising CDR2 as set forth in SEQ ID NO:36; and a region comprising CDR3 as set forth in SEQ ID NO:37. In embodiments, an amino acid in CDR1 comprises the moiety of Formula (II). In embodiments, an amino acid in CDR2 comprises the moiety of Formula (II). In embodiments, an amino acid in CDR3 comprises the moiety of Formula (II). In embodiments, the unnatural amino acid side chain of Formula (II) is at a position corresponding to position 4 in SEQ ID NO:37. In embodiments, the unnatural amino acid side chain of Formula (II) is capable of covalently binding to a lysine, tyrosine, or histidine on a viral spike (S) protein of a SARS-coronavirus. In embodiments, the unnatural amino acid side chain of Formula (II) is capable of covalently binding to a lysine, tyrosine, or histidine on a viral spike (S) protein of SARS-CoV-2. In embodiments, the unnatural amino acid side chain of Formula (II) is capable of covalently binding to a lysine on a viral spike (S) protein of SARS-CoV-2. In embodiments, the unnatural amino acid side chain of Formula (II) is capable of covalently binding to a tyrosine on a viral spike (S) protein of SARS-CoV-2. In embodiments, the unnatural amino acid side chain of Formula (II) is capable of covalently binding to a histidine on a viral spike (S) protein of SARS-CoV-2. In embodiments, the viral spike (S) protein of SARS-CoV-2 has at least 80% sequence identity to SEQ ID NO:5. In embodiments, the viral spike (S) protein of SARS-CoV-2 has at least 85% sequence identity to SEQ ID NO:5. In embodiments, the viral spike (S) protein of SARS-CoV-2 has at least 90% sequence identity to SEQ ID NO:5. In embodiments, the viral spike (S) protein of SARS-CoV-2 has at least 92% sequence identity to SEQ ID NO:5. In embodiments, the viral spike (S) protein of SARS-CoV-2 has at least 94% sequence identity to SEQ ID NO:5. In embodiments, the viral spike (S) protein of SARS-CoV-2 has at least 95% sequence identity to SEQ ID NO:5. In embodiments, the viral spike (S) protein of SARS-CoV-2 has at least 96% sequence identity to SEQ ID NO:5. In embodiments, the viral spike (S) protein of SARS-CoV-2 has at least 98% sequence identity to SEQ ID NO:5. In embodiments, the viral spike (S) protein of SARS-CoV-2 comprises SEQ ID NO:5. In embodiments, the viral spike (S) protein of SARS-CoV-2 has one or more mutations. In embodiments, the viral spike (S) protein of SARS-CoV-2 has one or more mutations comprising K417N, N439K, E484K, F490L, and N501Y. In embodiments, the SARS-CoV-2 spike protein has the amino acid sequence of the omicron variant or an omicron sub-variant (BA.1, BA.2, BA.3, BA.4, or BA.5).

The disclosure provides a single-domain antibody comprising an unnatural amino acid side chain; wherein the unnatural amino acid side chain is a moiety of Formula (II):

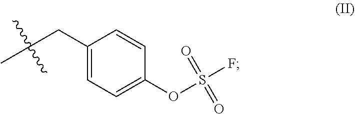 [SMILES: *Cc1ccc(OS(=O)(=O)F)cc1]

and wherein the single-domain antibody comprises an amino acid sequence having at least 75% sequence identity to SEQ ID NO:38. In embodiments, the single-domain antibody comprises an amino acid sequence having at least 80% sequence identity to SEQ ID NO:38. In embodiments, the single-domain antibody comprises an amino acid sequence having at least 85% sequence identity to SEQ ID NO:38. In embodiments, the single-domain antibody comprises an amino acid sequence having at least 90% sequence identity to SEQ ID NO:38. In embodiments, the single-domain antibody comprises an amino acid sequence having at least 92% sequence identity to SEQ ID NO:38. In embodiments, the single-domain antibody comprises an amino acid sequence having at least 94% sequence identity to SEQ ID NO:38. In embodiments, the single-domain antibody comprises an amino acid sequence having at least 95% sequence identity to SEQ ID NO:38. In embodiments, the single-domain antibody comprises an amino acid sequence having at least 96% sequence identity to SEQ ID NO:38. In embodiments, the single-domain antibody comprises an amino acid sequence having at least 98% sequence identity to SEQ ID NO:38. In embodiments, the single-domain antibody comprises an amino acid sequence of SEQ ID NO:38. In embodiments of the single-domain antibody having an amino acid sequence with at least 75%, 80%, 85%, 90%, 92%, 95%, 96%, or 98% sequence identity, the single-domain antibody has 100% sequence identity to CDR1, CDR2, and CDR3. In other words, variations in amino acid sequence identity (e.g., substitutions, deletions, additions) do not occur in CDR1 (i.e., the amino acids spanning positions 26 to 35 in SEQ ID NO:38), CDR2 (i.e., the amino acids spanning positions 50 to 59 in SEQ ID NO:38), and CDR3 (i.e., the amino acids spanning positions 98 to 116 in SEQ ID NO:38). In embodiments, the unnatural amino acid side chain of Formula (II) is at a position corresponding to position D115 or Y116 in SEQ ID NO:38. In embodiments, the unnatural amino acid side chain of Formula (II) is at a position corresponding to position D115 in SEQ ID NO:38. In embodiments, the unnatural amino acid side chain of Formula (II) is at a position corresponding to position Yl 16 in SEQ ID NO:38. In embodiments, the unnatural amino acid side chain of Formula (II) is capable of covalently binding to a lysine, tyrosine, or histidine on a viral spike (S) protein of SARS-CoV-2. In embodiments, the unnatural amino acid side chain of Formula (II) is capable of covalently binding to a lysine on a viral spike (S) protein of SARS-CoV-2. In embodiments, the unnatural amino acid side chain of Formula (II) is capable of covalently binding to a tyrosine on a viral spike (S) protein of SARS-CoV-2. In embodiments, the unnatural amino acid side chain of Formula (II) is capable of covalently binding to a histidine on a viral spike (S) protein of SARS-CoV-2. In embodiments, the viral spike (S) protein of SARS-CoV-2 has at least 80% sequence identity to SEQ ID NO:5. In embodiments, the viral spike (S) protein of SARS-CoV-2 has at least 85% sequence identity to SEQ ID NO:5. In embodiments, the viral spike (S) protein of SARS-CoV-2 has at least 90% sequence identity to SEQ ID NO:5. In embodiments, the viral spike (S) protein of SARS-CoV-2 has at least 92% sequence identity to SEQ ID NO:5. In embodiments, the viral spike (S) protein of SARS-CoV-2 has at least 94% sequence identity to SEQ ID NO:5. In embodiments, the viral spike (S) protein of SARS-CoV-2 has at least 95% sequence identity to SEQ ID NO:5. In embodiments, the viral spike (S) protein of SARS-CoV-2 has at least 96% sequence identity to SEQ ID NO:5. In embodiments, the viral spike (S) protein of SARS-CoV-2 has at least 98% sequence identity to SEQ ID NO:5. In embodiments, the viral spike (S) protein of SARS-CoV-2 comprises SEQ ID NO:5. In embodiments, the viral spike (S) protein of SARS-CoV-2 has one or more mutations. In embodiments, the viral spike (S) protein of SARS-CoV-2 has one or more mutations comprising K417N, N439K, E484K, F490L, and N501Y. In embodiments, the SARS-CoV-2 spike protein has the amino acid sequence of the omicron variant or an omicron sub-variant (BA.1, BA.2, BA.3, BA.4, or BA.5).

The disclosure provides a single-domain antibody comprising an unnatural amino acid side chain wherein the unnatural amino acid side chain is a moiety of Formula (II):

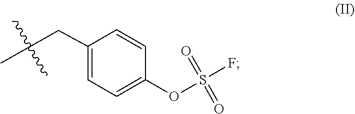 [SMILES: *Cc1ccc(OS(=O)(=O)F)cc1]

and wherein the single-domain antibody comprises a region comprising CDR1 as set forth in SEQ ID NO:39, a region comprising CDR2 as set forth in SEQ ID NO:40; and a region comprising CDR3 as set forth in SEQ ID NO:41. In embodiments, an amino acid in CDR1 comprises the moiety of Formula (II). In embodiments, an amino acid in CDR2 comprises the moiety of Formula (II). In embodiments, an amino acid in CDR3 comprises the moiety of Formula (II). In embodiments, the unnatural amino acid side chain of Formula (II) is at a position corresponding to position 18 or position 19 in SEQ ID NO:41. In embodiments, the unnatural amino acid side chain of Formula (II) is at a position corresponding to position 18 in SEQ ID NO:41. In embodiments, the unnatural amino acid side chain of Formula (II) is at a position corresponding to position 19 in SEQ ID NO:41. In embodiments, the unnatural amino acid side chain of Formula (II) is capable of covalently binding to a lysine, tyrosine, or histidine on a viral spike (S) protein of a SARS-coronavirus. In embodiments, the unnatural amino acid side chain of Formula (II) is capable of covalently binding to a lysine, tyrosine, or histidine on a viral spike (S) protein of SARS-CoV-2. In embodiments, the unnatural amino acid side chain of Formula (II) is capable of covalently binding to a lysine on a viral spike (S) protein of SARS-CoV-2. In embodiments, the unnatural amino acid side chain of Formula (II) is capable of covalently binding to a tyrosine on a viral spike (S) protein of SARS-CoV-2. In embodiments, the unnatural amino acid side chain of Formula (II) is capable of covalently binding to a histidine on a viral spike (S) protein of SARS-CoV-2. In embodiments, the viral spike (S) protein of SARS-CoV-2 has at least 80% sequence identity to SEQ ID NO:5. In embodiments, the viral spike (S) protein of SARS-CoV-2 has at least 85% sequence identity to SEQ ID NO:5. In embodiments, the viral spike (S) protein of SARS-CoV-2 has at least 90% sequence identity to SEQ ID NO:5. In embodiments, the viral spike (S) protein of SARS-CoV-2 has at least 92% sequence identity to SEQ ID NO:5. In embodiments, the viral spike (S) protein of SARS-CoV-2 has at least 94% sequence identity to SEQ ID NO:5. In embodiments, the viral spike (S) protein of SARS-CoV-2 has at least 95% sequence identity to SEQ ID NO:5. In embodiments, the viral spike (S) protein of SARS-CoV-2 has at least 96% sequence identity to SEQ ID NO:5. In embodiments, the viral spike (S) protein of SARS-CoV-2 has at least 98% sequence identity to SEQ ID NO:5. In embodiments, the viral spike (S) protein of SARS-CoV-2 comprises SEQ ID NO:5. In embodiments, the viral spike (S) protein of SARS-CoV-2 has one or more mutations. In embodiments, the viral spike (S) protein of SARS-CoV-2 has one or more mutations comprising K417N, N439K, E484K, F490L, and N501Y. In embodiments, the SARS-CoV-2 spike protein has the amino acid sequence of the omicron variant or an omicron sub-variant (BA.1, BA.2, BA.3, BA.4, or BA.5).

The disclosure provides a recombinant protein comprising an ACE2 receptor protein having an unnatural amino acid side chain; wherein the unnatural amino acid side chain is capable of covalently binding to lysine, tyrosine, or histidine. In aspects, the unnatural amino acid side chain is capable of covalently binding to lysine or tyrosine. In aspects, the unnatural amino acid side chain is capable of covalently binding to lysine. In aspects, the unnatural amino acid side chain is capable of covalently binding to tyrosine. In aspects, the unnatural amino acid side chain is capable of covalently binding to lysine, tyrosine, or histidine in a SARS-coronavirus. In aspects, the unnatural amino acid side chain is capable of covalently binding to lysine, tyrosine, or histidine in SARS-CoV-2. In aspects, the unnatural amino acid side chain is capable of covalently binding to lysine, tyrosine, or histidine in SARS-CoV-1. In aspects, the unnatural amino acid side chain is capable of covalently binding to lysine, tyrosine, or histidine in MERS-CoV.

In embodiments, the ACE2 receptor protein comprises any unnatural amino acid described herein. In embodiments, the ACE2 receptor protein comprises the unnatural amino acid of Formula (I). In embodiments, the ACE2 receptor protein comprises the unnatural amino acid of Formula (IA). In embodiments, the ACE2 receptor protein comprises the unnatural amino acid of Formula (IB). In embodiments, the ACE2 receptor protein comprises the unnatural amino acid of Formula (IC). In embodiments, the ACE2 receptor protein comprises the unnatural amino acid of Formula (ID). In embodiments, the ACE2 receptor protein comprises the unnatural amino acid of Formula (IE). In embodiments, the ACE2 receptor protein comprises the unnatural amino acid of Formula (IV). In embodiments, the ACE2 receptor protein comprises the unnatural amino acid of Formula (IVA). In embodiments, the ACE2 receptor protein comprises the unnatural amino acid of Formula (IVB). In embodiments, the ACE2 receptor protein comprises the unnatural amino acid of Formula (VII). In embodiments, the ACE2 receptor protein comprises the unnatural amino acid of Formula (VIIA). In embodiments, the ACE2 receptor protein comprises the unnatural amino acid of Formula (VIIB). In embodiments, the ACE2 receptor protein comprises the unnatural amino acid of Formula (VIIC). In embodiments, the ACE2 receptor protein comprises the unnatural amino acid of Formula (VIID). In embodiments, the ACE2 receptor protein comprises the unnatural amino acid of Formula (IVB). In embodiments, the ACE2 receptor protein comprises an unnatural amino acid, wherein the unnatural amino acid comprises a side chain as described herein. In embodiments, the ACE2 receptor protein comprises an unnatural amino acid, wherein the unnatural amino acid comprises a side chain of Formula (II). In embodiments, the ACE2 receptor protein comprises an unnatural amino acid, wherein the unnatural amino acid comprises a side chain of Formula (V). In embodiments, the ACE2 receptor protein comprises an unnatural amino acid, wherein the unnatural amino acid comprises a side chain of Formula (IE-A). In embodiments, the ACE2 receptor protein comprises an unnatural amino acid, wherein the unnatural amino acid comprises a side chain of Formula (VA). In embodiments, the ACE2 receptor protein comprises an unnatural amino acid, wherein the unnatural amino acid comprises a side chain of Formula (VIIIC). In embodiments, the ACE2 receptor protein comprises an unnatural amino acid, wherein the unnatural amino acid comprises a side chain of Formula (VB).

In embodiments, the unnatural amino acid residue having an unnatural amino acid side chain that is capable of covalently binding to lysine, tyrosine, or histidine is FSY. In embodiments, the unnatural amino acid side chain of FSY that is capable of covalently binding to lysine, tyrosine, or histidine is a moiety of Formula (II):

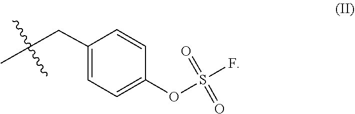 [SMILES: *Cc1ccc(OS(=O)(=O)F)cc1]

In embodiments, the ACE2 receptor protein is a soluble extracellular domain of the human ACE2 receptor protein. In aspects, the ACE2 receptor protein comprises SEQ ID NO:1. In aspects, the ACE2 receptor protein further comprises an Fc fragment, an epitope tag, or a combination thereof. In aspects, the ACE2 receptor protein further comprises an Fc fragment. In aspects, the Fc fragment is an IgG Fc fragment. In aspects, the Fc fragment is a human IgG Fc fragment. In aspects, the Fc fragment is an IgG1 Fc fragment. In aspects, the Fc fragment is a human IgG1 Fc fragment. In aspects, the human IgG1 Fc fragment comprises an amino acid sequence having at least 80% sequence identity, at least 85% sequence identity, at least 90% sequence identity, at least 95% sequence identity, or 100% sequence identity to the IgG Fc fragment having the amino acid sequence identified by UniProtKB Reference No. P01857. In aspects, the IgG1 Fc fragment comprises an amino acid sequence having at least 80% sequence identity, at least 85% sequence identity, at least 90% sequence identity, at least 95% sequence identity, or 100% sequence identity to the IgG Fc fragment having the amino acid sequence identified by UniProtKB Reference No. P01868. In aspects, the human IgG1 Fc fragment comprises an amino acid sequence having at least 80% sequence identity, at least 85% sequence identity, at least 90% sequence identity, at least 95% sequence identity, or 100% sequence identity to the IgG Fc fragment having the amino acid sequence identified by UniProtKB Reference No. P0DOX5. In aspects, the epitope tag is HA, HIS, FLAG, AU1, AU5, Myc, Glu-Glu, OLLAS, T7, V5, VSV-G, E-Tag, S-Tag, Avi, HSV, KT3, TK15, GST, or Strep-tag II. In aspects, the epitope tag is a polyhistidine (HIS) tag. In aspects, the epitope tag is a polyhistidine tag comprising 6 histidine residues.

In aspects, the ACE2 receptor protein has at least 75% sequence identity to SEQ ID NO:1. In aspects, the ACE2 receptor protein has at least 80% sequence identity to SEQ ID NO:1. In aspects, the ACE2 receptor protein has at least 85% sequence identity to SEQ ID NO:1. In aspects, the ACE2 receptor protein has at least 90% sequence identity to SEQ ID NO:1. In aspects, the ACE2 receptor protein has at least 92% sequence identity to SEQ ID NO:1. In aspects, the ACE2 receptor protein has at least 94% sequence identity to SEQ ID NO:1. In aspects, the ACE2 receptor protein has at least 95% sequence identity to SEQ ID NO:1. In aspects, the ACE2 receptor protein has at least 96% sequence identity to SEQ ID NO:1. In aspects, the ACE2 receptor protein has at least 98% sequence identity to SEQ ID NO:1. In aspects, the ACE2 receptor protein is SEQ ID NO:1. In aspects, the ACE receptor protein of SEQ ID NO:1 comprises the unnatural amino acid side chain of Formula (II) at a position corresponding to position 30, 34, 37, 38, 42, or 83. In aspects, the ACE receptor protein of SEQ ID NO:1 comprises the unnatural amino acid side chain of Formula (II) at a position corresponding to position 30, 34, 37, or 42. In aspects, the ACE receptor protein of SEQ ID NO:1 comprises the unnatural amino acid side chain of Formula (II) at a position corresponding to position 34, 37, or 42. In aspects, the ACE receptor protein of SEQ ID NO:1 comprises the unnatural amino acid side chain of Formula (II) at a position corresponding to position 34. When the ACE receptor protein of SEQ ID NO:1 comprises the unnatural amino acid side chain of Formula (II) at position corresponding to position 34, the ACE receptor protein is SEQ ID NO:2. In aspects, the ACE receptor protein of SEQ ID NO:1 comprises the unnatural amino acid side chain of Formula (II) at a position corresponding to position 37. When the ACE receptor protein of SEQ ID NO:1 comprises the unnatural amino acid side chain of Formula (II) at position corresponding to 37, the ACE receptor protein is SEQ ID NO:3. In aspects, the ACE receptor protein of SEQ ID NO:1 comprises the unnatural amino acid side chain of Formula (II) at a position corresponding to position 42. When the ACE receptor protein of SEQ ID NO:1 comprises the unnatural amino acid side chain of Formula (II) at position corresponding to position 42, the ACE receptor protein is SEQ ID NO:4.

In aspects, the ACE2 receptor protein has at least 75% sequence identity to the region spanning amino acid residue 19 to amino acid residue 615 in SEQ ID NO:1. In aspects, the ACE2 receptor protein has at least 80% sequence identity to the region spanning amino acid residue 19 to amino acid residue 615 in SEQ ID NO:1. In aspects, the ACE2 receptor protein has at least 85% sequence identity to the region spanning amino acid residue 19 to amino acid residue 615 in SEQ ID NO:1. In aspects, the ACE2 receptor protein has at least 90% sequence identity to the region spanning amino acid residue 19 to amino acid residue 615 in SEQ ID NO:1. In aspects, the ACE2 receptor protein has at least 92% sequence identity to the region spanning amino acid residue 19 to amino acid residue 615 in SEQ ID NO:1. In aspects, the ACE2 receptor protein has at least 94% sequence identity to the region spanning amino acid residue 19 to amino acid residue 615 in SEQ ID NO:1. In aspects, the ACE2 receptor protein has at least 95% sequence identity to the region spanning amino acid residue 19 to amino acid residue 615 in SEQ ID NO:1. In aspects, the ACE2 receptor protein has at least 96% sequence identity to the region spanning amino acid residue 19 to amino acid residue 615 in SEQ ID NO:1. In aspects, the ACE2 receptor protein has at least 98% sequence identity to the region spanning amino acid residue 19 to amino acid residue 615 in SEQ ID NO:1. In aspects, the ACE2 receptor protein is the region spanning amino acid residue 19 to amino acid residue 615 in SEQ ID NO:1. In aspects, the ACE receptor protein having an amino acid sequence spanning amino acid residue 19 to amino acid residue 615 in of SEQ ID NO:1 comprises the unnatural amino acid side chain of Formula (II) at a position corresponding to position 30, 34, 37, 38, 42, or 83. In aspects, the ACE receptor protein having an amino acid sequence spanning amino acid residue 19 to amino acid residue 615 in of SEQ ID NO:1 comprises the unnatural amino acid side chain of Formula (II) at a position corresponding to position 30, 34, 37, or 42. In aspects, the ACE receptor protein having an amino acid sequence spanning amino acid residue 19 to amino acid residue 615 in of SEQ ID NO:1 comprises the unnatural amino acid side chain of Formula (II) at a position corresponding to position 34, 37, or 42. In aspects, the ACE receptor protein having an amino acid sequence spanning amino acid residue 19 to amino acid residue 615 in of SEQ ID NO:1 comprises the unnatural amino acid side chain of Formula (II) at a position corresponding to position 34. In aspects, the ACE receptor protein having an amino acid sequence spanning amino acid residue 19 to amino acid residue 615 in of SEQ ID NO:1 comprises the unnatural amino acid side chain of Formula (II) at position corresponding to 37. In aspects, the ACE receptor protein having an amino acid sequence spanning amino acid residue 19 to amino acid residue 615 in of SEQ ID NO:1 comprises the unnatural amino acid side chain of Formula (II) at a position corresponding to position 42.

In embodiments, the disclosure provides protein complexes. In aspects, the protein complexes comprise two or more proteins. In aspects, the protein complexes comprise two proteins. In aspects, the protein complex comprises the recombinant protein described herein linked to a SARS-coronavirus. In aspects, the protein complex comprises the recombinant protein described herein covalently bonded to a SARS-coronavirus. In aspects, the protein complex comprises the recombinant protein described herein covalently bonded to a viral spike (S) protein on a SARS-coronavirus. In aspects, the protein complex comprises the recombinant protein described herein covalently bonded to lysine, tyrosine, or histidine on a viral spike (S) protein on a SARS-coronavirus. In aspects, the protein complex comprises the recombinant protein described herein covalently bonded to lysine or tyrosine on a viral spike (S) protein on a SARS-coronavirus. In aspects, the protein complex comprises the recombinant protein described herein covalently bonded to lysine on a viral spike (S) protein on a SARS-coronavirus. In aspects, the protein complex comprises the recombinant protein described herein covalently bonded to tyrosine on a viral spike (S) protein on a SARS-coronavirus. In aspects, the SARS-coronavirus is SARS-CoV-1. In aspects, the SARS-coronavirus is SARS-CoV-2. In aspects, the SARS-coronavirus is MERS-CoV. In embodiments, the disclosure provides a SARS-coronavirus comprising the protein complex described herein. In aspects, SARS-CoV-1 comprises the protein complex described herein. In aspects, SARS-CoV-2 comprises the protein complex described herein. In aspects, MERS-CoV comprises the protein complex described herein.

The protein complexes herein comprise any SARS-coronavirus. In aspects, the protein complexes comprise a viral spike (S) protein of a SARS-coronavirus. In aspects, the SARS-coronavirus is SARS-CoV-1. In aspects, the SARS-coronavirus is SARS-CoV-2. the SARS-coronavirus is MERS-CoV. In aspects, the viral spike (S) protein of the SARS-CoV has at least 50% sequence identity to SEQ ID NO:5. In aspects, the viral spike (S) protein of the SARS-CoV has at least 55% sequence identity to SEQ ID NO:5. In aspects, the viral spike (S) protein of the SARS-CoV has at least 60% sequence identity to SEQ ID NO:5. In aspects, the viral spike (S) protein of the SARS-CoV has at least 65% sequence identity to SEQ ID NO:5. In aspects, the viral spike (S) protein of the SARS-CoV has at least 70% sequence identity to SEQ ID NO:5. In aspects, the viral spike (S) protein of the SARS-CoV has at least 75% sequence identity to SEQ ID NO:5. In aspects, the viral spike (S) protein of the SARS-CoV has at least 80% sequence identity to SEQ ID NO:5. In aspects, the viral spike (S) protein of the SARS-CoV has at least 85% sequence identity to SEQ ID NO:5. In aspects, the viral spike (S) protein of the SARS-CoV has at least 90% sequence identity to SEQ ID NO:5. In aspects, the viral spike (S) protein of the SARS-CoV has at least 95% sequence identity to SEQ ID NO:5. In aspects, the viral spike (S) protein of the SARS-CoV is SEQ ID NO:5. In aspects, the viral spike (S) protein of the SARS-CoV is a conservatively modified variant of SEQ ID NO:5. In aspects, the viral spike (S) protein of the SARS-CoV is any SARS-CoV that comprises a tyrosine residue at a position corresponding to position 489, 453, 505, or 449 in SEQ ID NO:5; or that comprises a lysine at a position corresponding to position 417. In aspects, the viral spike (S) protein of the SARS-CoV is any SARS-CoV that comprises a tyrosine residue at a position corresponding to position 453, 505, or 449 in SEQ ID NO:5. In aspects, the viral spike (S) protein of the SARS-CoV is any SARS-CoV that comprises a tyrosine residue at a position corresponding to position 453 in SEQ ID NO:5. In aspects, the viral spike (S) protein of the SARS-CoV is any SARS-CoV that comprises a tyrosine residue at a position corresponding to position 505 in SEQ ID NO:5. In aspects, the viral spike (S) protein of the SARS-CoV is any SARS-CoV that comprises a tyrosine residue at a position corresponding to position 449 in SEQ ID NO:5. In embodiments, the viral spike (S) protein of the SARS-CoV has one or more mutations. In embodiments, the viral spike (S) protein of SEQ ID NO:5 comprises one or more mutations. In embodiments, the viral spike (S) protein of SEQ ID NO:5 comprises at least one mutation selected from the group consisting of K417N, N439K, E484K, F490L, and N501Y. In embodiments, the viral spike (S) protein of SEQ ID NO:5 comprises mutation K417N. In embodiments, the viral spike (S) protein of SEQ ID NO:5 comprises mutation N439K. In embodiments, the viral spike (S) protein of SEQ ID NO:5 comprises mutation E484K. In embodiments, the viral spike (S) protein of SEQ ID NO:5 comprises mutation F490L. In embodiments, the viral spike (S) protein of SEQ ID NO:5 comprises mutation N501Y. In embodiments, the viral spike (S) protein of SEQ ID NO:5 comprises mutations K417N, E484K, and N501Y. In embodiments, the SARS-CoV-2 spike protein has the amino acid sequence of the omicron variant or an omicron sub-variant (BA.1, BA.2, BA.3, BA.4, or BA.5). In embodiments, the disclosure provides a SARS-coronavirus comprising the protein complex described herein. In aspects, SARS-CoV-1 comprises the protein complex described herein. In aspects, SARS-CoV-2 comprises the protein complex described herein. In aspects, MERS-CoV comprises the protein complex described herein.

As described herein, the unnatural amino acid side of Formula (II) is capable of binding to a lysine, tyrosine, or histidine residue in SARS-CoV. In aspects, the unnatural amino acid side of Formula (II) is capable of binding to a lysine, tyrosine, or histidine residue in SARS-CoV. In aspects, the unnatural amino acid side of Formula (II) is capable of binding to a lysine residue at a position corresponding to K417 in SARS-CoV of SEQ ID NO:5. In aspects, the unnatural amino acid side of Formula (II) is capable of binding to a tyrosine residue at a position corresponding to Y453, Y505, Y449, or Y489 in SARS-CoV of SEQ ID NO:5. In aspects, the unnatural amino acid side of Formula (II) is capable of binding to a tyrosine residue at a position corresponding to Y453, Y505, or Y449 in SARS-CoV in SEQ ID NO:5. In aspects, the unnatural amino acid side of Formula (II) is capable of binding to a tyrosine residue at a position corresponding to Y453 in SARS-CoV in SEQ ID NO:5. In aspects, the unnatural amino acid side of Formula (II) is capable of binding to a tyrosine residue at a position corresponding to Y505 in SARS-CoV in SEQ ID NO:5. In aspects, the unnatural amino acid side of Formula (II) is capable of binding to a tyrosine residue at a position corresponding to Y449 in SARS-CoV in SEQ ID NO:5. SEQ ID NO:5 comprises the viral spike (S) protein of SARS-CoV. In embodiments, the SARS-CoV has one or more mutations. In embodiments, SEQ ID NO:5 comprises at least one mutation selected from the group consisting of K417N, N439K, E484K, F490L, and N501Y. In embodiments, SEQ ID NO:5 comprises mutation K417N. In embodiments, SEQ ID NO:5 comprises mutation N439K. In embodiments, SEQ ID NO:5 comprises mutation E484K. In embodiments, SEQ ID NO:5 comprises mutation F490L. In embodiments, SEQ ID NO:5 comprises mutation N501Y. In embodiments, SEQ ID NO:5 comprises mutations K417N, E484K, and N501Y. In embodiments, the SARS-CoV-2 spike protein has the amino acid sequence of the omicron variant or an omicron sub-variant (BA.1, BA.2, BA.3, BA.4, or BA.5). In embodiments, the disclosure provides a SARS-coronavirus comprising the protein complex described herein. In aspects, SARS-CoV-1 comprises the protein complex described herein. In aspects, SARS-CoV-2 comprises the protein complex described herein. In aspects, MERS-CoV comprises the protein complex described herein.

In embodiments, the protein complex comprises a recombinant protein described herein having an ACE2 receptor protein which comprises the unnatural amino acid side chain of Formula (II) at a position corresponding to position 34 in SEQ ID NO:1 that is covalently bonded via the moiety of Formula (II) to a lysine residue of a viral spike (S) protein of SARS-CoV at a position corresponding to position 453, 505, 449, or 489 in SEQ ID NO:5. In aspects, the ACE2 receptor protein comprises the unnatural amino acid side chain of Formula (II) at a position corresponding to position 34 in SEQ ID NO:1 that is covalently bonded via the moiety of Formula (II) to a lysine residue of a viral spike (S) protein of SARS-CoV at a position corresponding to position 453 in SEQ ID NO:5. In aspects, the ACE2 receptor protein comprises the unnatural amino acid side chain of Formula (II) at a position corresponding to position 34 in SEQ ID NO:1 that is covalently bonded via the moiety of Formula (II) to a lysine residue of a viral spike (S) protein of SARS-CoV at a position corresponding to position 505 in SEQ ID NO:5. In aspects, the ACE2 receptor protein comprises the unnatural amino acid side chain of Formula (II) at a position corresponding to position 34 in SEQ ID NO:1 that is covalently bonded via the moiety of Formula (II) to a lysine residue of a viral spike (S) protein of SARS-CoV at a position corresponding to position 449 in SEQ ID NO:5. In aspects, the ACE2 receptor protein comprises the unnatural amino acid side chain of Formula (II) at a position corresponding to position 34 in SEQ ID NO:1 that is covalently bonded via the moiety of Formula (II) to a lysine residue of a viral spike (S) protein of SARS-CoV at a position corresponding to position 489 in SEQ ID NO:5. In embodiments, the SARS-CoV has one or more mutations. In embodiments, SEQ ID NO:5 comprises at least one mutation selected from the group consisting of K417N, N439K, E484K, F490L, and N501Y. In embodiments, SEQ ID NO:5 comprises mutation K417N. In embodiments, SEQ ID NO:5 comprises mutation N439K. In embodiments, SEQ ID NO:5 comprises mutation E484K. In embodiments, SEQ ID NO:5 comprises mutation F490L. In embodiments, SEQ ID NO:5 comprises mutation N501Y. In embodiments, SEQ ID NO:5 comprises mutations K417N, E484K, and N501Y. In embodiments, the SARS-CoV-2 spike protein has the amino acid sequence of the omicron variant or an omicron sub-variant (BA.1, BA.2, BA.3, BA.4, or BA.5). In embodiments, the disclosure provides a SARS-coronavirus comprising the protein complex described herein. In aspects, SARS-CoV-1 comprises the protein complex described herein. In aspects, SARS-CoV-2 comprises the protein complex described herein. In aspects, MERS-CoV comprises the protein complex described herein.

In embodiments, the protein complex comprises a recombinant protein described herein having an ACE2 receptor protein which comprises the unnatural amino acid side chain of Formula (II) at a position corresponding to position 37 in SEQ ID NO:1 that is covalently bonded via the moiety of Formula (II) to a lysine residue of a viral spike (S) protein of SARS-CoV at a position corresponding to position 453, 505, 449, or 489 in SEQ ID NO:5. In aspects, the ACE2 receptor protein comprises the unnatural amino acid side chain of Formula (II) at a position corresponding to position 37 in SEQ ID NO:1 that is covalently bonded via the moiety of Formula (II) to a lysine residue of a viral spike (S) protein of SARS-CoV at a position corresponding to position 453 in SEQ ID NO:5. In aspects, the ACE2 receptor protein comprises the unnatural amino acid side chain of Formula (II) at a position corresponding to position 37 in SEQ ID NO:1 that is covalently bonded via the moiety of Formula (II) to a lysine residue of a viral spike (S) protein of SARS-CoV at a position corresponding to position 505 in SEQ ID NO:5. In aspects, the ACE2 receptor protein comprises the unnatural amino acid side chain of Formula (II) at a position corresponding to position 37 in SEQ ID NO:1 that is covalently bonded via the moiety of Formula (II) to a lysine residue of a viral spike (S) protein of SARS-CoV at a position corresponding to position 449 in SEQ ID NO:5. In aspects, the ACE2 receptor protein comprises the unnatural amino acid side chain of Formula (II) at a position corresponding to position 37 in SEQ ID NO:1 that is covalently bonded via the moiety of Formula (II) to a lysine residue of a viral spike (S) protein of SARS-CoV at a position corresponding to position 489 in SEQ ID NO:5. In embodiments, the viral spike (S) protein of SEQ ID NO:5 comprises one or more mutations. In embodiments, the viral spike (S) protein of SEQ ID NO:5 comprises at least one mutation selected from the group consisting of K417N, N439K, E484K, F490L, and N501Y. In embodiments, the viral spike (S) protein of SEQ ID NO:5 comprises mutation K417N. In embodiments, the viral spike (S) protein of SEQ ID NO:5 comprises mutation N439K. In embodiments, the viral spike (S) protein of SEQ ID NO:5 comprises mutation E484K. In embodiments, the viral spike (S) protein of SEQ ID NO:5 comprises mutation F490L. In embodiments, the viral spike (S) protein of SEQ ID NO:5 comprises mutation N501Y. In embodiments, the viral spike (S) protein of SEQ ID NO:5 comprises mutations K417N, E484K, and N501Y. In embodiments, the SARS-CoV-2 spike protein has the amino acid sequence of the omicron variant or an omicron sub-variant (BA.1, BA.2, BA.3, BA.4, or BA.5). In embodiments, the disclosure provides a SARS-coronavirus comprising the protein complex described herein. In aspects, SARS-CoV-1 comprises the protein complex described herein. In aspects, SARS-CoV-2 comprises the protein complex described herein. In aspects, MERS-CoV comprises the protein complex described herein.

In embodiments, the protein complex comprises a recombinant protein described herein having an ACE2 receptor protein which comprises the unnatural amino acid side chain of Formula (II) at a position corresponding to position 42 in SEQ ID NO:1 that is covalently bonded via the moiety of Formula (II) to a lysine residue of a viral spike (S) protein of SARS-CoV at a position corresponding to position 453, 505, 449, or 489 in SEQ ID NO:5. In aspects, the ACE2 receptor protein comprises the unnatural amino acid side chain of Formula (II) at a position corresponding to position 42 in SEQ ID NO:1 that is covalently bonded via the moiety of Formula (II) to a lysine residue of a viral spike (S) protein of SARS-CoV at a position corresponding to position 453 in SEQ ID NO:5. In aspects, the ACE2 receptor protein comprises the unnatural amino acid side chain of Formula (II) at a position corresponding to position 42 in SEQ ID NO:1 that is covalently bonded via the moiety of Formula (II) to a lysine residue of a viral spike (S) protein of SARS-CoV at a position corresponding to position 505 in SEQ ID NO:5. In aspects, the ACE2 receptor protein comprises the unnatural amino acid side chain of Formula (II) at a position corresponding to position 42 in SEQ ID NO:1 that is covalently bonded via the moiety of Formula (II) to a lysine residue of a viral spike (S) protein of SARS-CoV at a position corresponding to position 449 in SEQ ID NO:5. In aspects, the ACE2 receptor protein comprises the unnatural amino acid side chain of Formula (II) at a position corresponding to position 42 in SEQ ID NO:1 that is covalently bonded via the moiety of Formula (II) to a lysine residue of a viral spike (S) protein of SARS-CoV at a position corresponding to position 489 in SEQ ID NO:5. In embodiments, the viral spike (S) protein of SEQ ID NO:5 comprises one or more mutations. In embodiments, the viral spike (S) protein of SEQ ID NO:5 comprises at least one mutation selected from the group consisting of K417N, N439K, E484K, F490L, and N501Y. In embodiments, the viral spike (S) protein of SEQ ID NO:5 comprises mutation K417N. In embodiments, the viral spike (S) protein of SEQ ID NO:5 comprises mutation N439K. In embodiments, the viral spike (S) protein of SEQ ID NO:5 comprises mutation E484K. In embodiments, the viral spike (S) protein of SEQ ID NO:5 comprises mutation F490L. In embodiments, the viral spike (S) protein of SEQ ID NO:5 comprises mutation N501Y. In embodiments, the viral spike (S) protein of SEQ ID NO:5 comprises mutations K417N, E484K, and N501Y. In embodiments, the SARS-CoV-2 spike protein has the amino acid sequence of the omicron variant or an omicron sub-variant (BA.1, BA.2, BA.3, BA.4, or BA.5). In embodiments, the disclosure provides a SARS-coronavirus comprising the protein complex described herein. In aspects, SARS-CoV-1 comprises the protein complex described herein. In aspects, SARS-CoV-2 comprises the protein complex described herein. In aspects, MERS-CoV comprises the protein complex described herein.

The disclosure provides cells comprising the compositions (e.g., single-domain antibodies, recombinant proteins) and complexes (e.g., single-domain antibody-SARS-CoV or recombinant protein-SARS-CoV) provided herein, including embodiments thereof.

In embodiments, the disclosure provides a cell comprising the single-domain antibody described herein. In aspects, the cell further includes a vector as described herein. In embodiments, the single-domain antibody is biosynthesized inside the cell, thereby generating a cell containing the single-domain antibody. In aspects, the single-domain antibody is contained in the medium outside the cell and penetrates into the cell, thereby generating a cell containing the single-domain antibody. In aspects, the cell comprises a protein complex described herein. In aspects, the cell comprises a SARS-CoV comprising the protein complex described herein. In aspects, the cell comprises a single-domain antibody that is synthesized inside the cell. In aspects, the cell comprises a single-domain antibody that is synthesized outside a cell, and that penetrates into the cell. A cell can be any prokaryotic or eukaryotic cell. For example, any of the compounds (e.g., single-domain antibody) compositions described herein can be expressed in bacterial cells such as E. coli, insect cells, yeast or mammalian cells (such as Hela cells, Chinese hamster ovary cells (CHO) or COS cells). In aspects, a cell can be a premature mammalian cell, i.e., pluripotent stem cell. In aspects, a cell can be derived from other human tissue. Other suitable cells are known to those skilled in the art.

The single-domain antibody provided herein may be delivered to cells using methods well known in the art. Thus, in an aspect is provided a nucleic acid sequence encoding the single-domain antibody described herein, including embodiments and aspects thereof. Thus, in an aspect is provided a vector including a nucleic acid sequence encoding the single-domain antibody described herein, including embodiments and aspects thereof.

In embodiments, the disclosure provides a nucleic acid comprising SEQ ID NO:42. In embodiments, the disclosure provides a nucleic acid as set forth in SEQ ID NO:42. In embodiments, the nucleic acid has at least 75% sequence identity to SEQ ID NO:42. In embodiments, the nucleic acid has at least 80% sequence identity to SEQ ID NO:42. In embodiments, the nucleic acid has at least 85% sequence identity to SEQ ID NO:42. In embodiments, the nucleic acid has at least 90% sequence identity to SEQ ID NO:42. In embodiments, the nucleic acid has at least 92% sequence identity to SEQ ID NO:42. In embodiments, the nucleic acid has at least 94% sequence identity to SEQ ID NO:42. In embodiments, the nucleic acid has at least 95% sequence identity to SEQ ID NO:42. In embodiments, the nucleic acid has at least 96% sequence identity to SEQ ID NO:42. In embodiments, the nucleic acid has at least 98% sequence identity to SEQ ID NO:42.

In embodiments, the disclosure provides a nucleic acid comprising SEQ ID NO:43. In embodiments, the disclosure provides a nucleic acid as set forth in SEQ ID NO:43. In embodiments, the nucleic acid has at least 75% sequence identity to SEQ ID NO:43. In embodiments, the nucleic acid has at least 80% sequence identity to SEQ ID NO:43. In embodiments, the nucleic acid has at least 85% sequence identity to SEQ ID NO:43. In embodiments, the nucleic acid has at least 90% sequence identity to SEQ ID NO:43. In embodiments, the nucleic acid has at least 92% sequence identity to SEQ ID NO:43. In embodiments, the nucleic acid has at least 94% sequence identity to SEQ ID NO:43. In embodiments, the nucleic acid has at least 95% sequence identity to SEQ ID NO:43. In embodiments, the nucleic acid has at least 96% sequence identity to SEQ ID NO:43. In embodiments, the nucleic acid has at least 98% sequence identity to SEQ ID NO:43.

In embodiments, the disclosure provides a nucleic acid comprising SEQ ID NO:44. In embodiments, the disclosure provides a nucleic acid as set forth in SEQ ID NO:44. In embodiments, the nucleic acid has at least 75% sequence identity to SEQ ID NO:44. In embodiments, the nucleic acid has at least 80% sequence identity to SEQ ID NO:44. In embodiments, the nucleic acid has at least 85% sequence identity to SEQ ID NO:44. In embodiments, the nucleic acid has at least 90% sequence identity to SEQ ID NO:44. In embodiments, the nucleic acid has at least 92% sequence identity to SEQ ID NO:44. In embodiments, the nucleic acid has at least 94% sequence identity to SEQ ID NO:44. In embodiments, the nucleic acid has at least 95% sequence identity to SEQ ID NO:44. In embodiments, the nucleic acid has at least 96% sequence identity to SEQ ID NO:44. In embodiments, the nucleic acid has at least 98% sequence identity to SEQ ID NO:44.

In embodiments, the disclosure provides a cell comprising the recombinant protein described herein. In aspects, the cell further includes a vector as described herein. In embodiments, the recombinant protein is biosynthesized inside the cell, thereby generating a cell containing the recombinant protein. In aspects, the recombinant protein is contained in the medium outside the cell and penetrates into the cell, thereby generating a cell containing the recombinant protein. In aspects, the cell comprises a protein complex described herein. In aspects, the cell comprises a SARS-CoV comprising the protein complex described herein. In aspects, the cell comprises a recombinant protein that is synthesized inside the cell. In aspects, the cell comprises a recombinant protein that is synthesized outside a cell, and that penetrates into the cell. A cell can be any prokaryotic or eukaryotic cell. For example, any of the compounds (e.g., recombinant proteins) and compositions described herein can be expressed in bacterial cells such as E. coli, insect cells, yeast or mammalian cells (such as Hela cells, Chinese hamster ovary cells (CHO) or COS cells). In aspects, a cell can be a premature mammalian cell, i.e., pluripotent stem cell. In aspects, a cell can be derived from other human tissue. Other suitable cells are known to those skilled in the art.

The recombinant protein provided herein may be delivered to cells using methods well known in the art. Thus, in an aspect is provided a nucleic acid sequence encoding the recombinant protein described herein, including embodiments and aspects thereof. Thus, in an aspect is provided a vector including a nucleic acid sequence encoding the recombinant protein described herein, including embodiments and aspects thereof.

The disclosure provides a nucleic acid having at least 80% sequence identity to SEQ ID NO:21, SEQ ID NO:23, SEQ ID NO:24, SEQ ID NO:25, SEQ ID NO:27, or SEQ ID NO:28. In embodiments, the nucleic acid has at least 85% sequence identity to SEQ ID NO:21, SEQ ID NO:23, SEQ ID NO:24, SEQ ID NO:25, SEQ ID NO:27, or SEQ ID NO:28. In embodiments, the nucleic acid has at least 90% sequence identity to SEQ ID NO:21, SEQ ID NO:23, SEQ ID NO:24, SEQ ID NO:25, SEQ ID NO:27, or SEQ ID NO:28. In embodiments, the nucleic acid has at least 92% sequence identity to SEQ ID NO:21, SEQ ID NO:23, SEQ ID NO:24, SEQ ID NO:25, SEQ ID NO:27, or SEQ ID NO:28. In embodiments, the nucleic acid has at least 94% sequence identity to SEQ ID NO:21, SEQ ID NO:23, SEQ ID NO:24, SEQ ID NO:25, SEQ ID NO:27, or SEQ ID NO:28. In embodiments, the nucleic acid has at least 95% sequence identity to SEQ ID NO:21, SEQ ID NO:23, SEQ ID NO:24, SEQ ID NO:25, SEQ ID NO:27, or SEQ ID NO:28. In embodiments, the nucleic acid has at least 96% sequence identity to SEQ ID NO:21, SEQ ID NO:23, SEQ ID NO:24, SEQ ID NO:25, SEQ ID NO:27, or SEQ ID NO:28. In embodiments, the nucleic acid has at least 98% sequence identity to SEQ ID NO:21, SEQ ID NO:23, SEQ ID NO:24, SEQ ID NO:25, SEQ ID NO:27, or SEQ ID NO:28. In embodiments, the nucleic acid is SEQ ID NO:21. In embodiments, the nucleic acid is SEQ ID NO:23. In embodiments, the nucleic acid is SEQ ID NO:24. In embodiments, the nucleic acid is SEQ ID NO:25. In embodiments, the nucleic acid is SEQ ID NO:27. In embodiments, the nucleic acid is SEQ ID NO:28.

### Cellular Compositions

The disclosure provides cells comprising the compounds, compositions and complexes provided herein, including embodiments thereof. In embodiments, a cell comprise the compound of Formula (I), including any embodiment thereof. In embodiments, a cell comprise the compound of Formula (II), including any embodiment thereof. In embodiments, a cell comprise the compound of Formula (III), including any embodiment thereof. In embodiments, a cell comprise the compound of Formula (IV), including any embodiment thereof. In embodiments, a cell comprise the compound of Formula (V), including any embodiment thereof. In embodiments, a cell comprise the compound of Formula (VI), including any embodiment thereof. In embodiments, a cell comprise the compound of Formula (VII), including any embodiment thereof. In embodiments, a cell comprise the compound of Formula (VIII), including any embodiment thereof. In embodiments, a cell comprise the compound of Formula (IX), including any embodiment thereof. In embodiments, a cell comprise the compound of Formula (X), including any embodiment thereof. In embodiments, a cell comprise the compound of Formula (XI), including any embodiment thereof.

In embodiments, the cell further includes a mutant pyrrolysyl-tRNA synthetase as described herein, including embodiments thereof. In embodiments, the cell further includes a vector as described herein, including embodiments thereof. In embodiments, the cell further includes a tRNA^{Pyl}.

In embodiments, the compound of Formula (I) (including embodiments thereof) is biosynthesized inside the cell, thereby generating a cell containing the compound of Formula (I). In embodiments, the compound of Formula (I) is contained in the medium outside the cell and penetrates into the cell, thereby generating a cell containing the compound of Formula (I). In embodiments, the cell comprises the compound of Formula (II) (including embodiments thereof). In embodiments, the cell comprises the compound of Formula (II) that is synthesized inside the cell. In embodiments, the cell comprises the compound of Formula (II) that is synthesized outside a cell, and that penetrates into the cell.

In embodiments, the compound of Formula (IV) (including embodiments thereof) is biosynthesized inside the cell, thereby generating a cell containing the compound of Formula (IV). In embodiments, the compound of Formula (IV) is contained in the medium outside the cell and penetrates into the cell, thereby generating a cell containing the compound of Formula (IV). In embodiments, the cell comprises the compound of Formula (V) (including embodiments thereof). In embodiments, the cell comprises the compound of Formula (V) that is synthesized inside the cell. In embodiments, the cell comprises the compound of Formula (V) that is synthesized outside a cell, and that penetrates into the cell.

In embodiments, the compound of Formula (VII) (including embodiments thereof) is biosynthesized inside the cell, thereby generating a cell containing the compound of Formula (VII). In embodiments, the compound of Formula (VII) is contained in the medium outside the cell and penetrates into the cell, thereby generating a cell containing the compound of Formula (VII). In embodiments, the cell comprises the compound of Formula (VIII) (including embodiments thereof). In embodiments, the cell comprises the compound of Formula (VIII) that is synthesized inside the cell. In embodiments, the cell comprises the compound of Formula (VIII) that is synthesized outside a cell, and that penetrates into the cell.

In embodiments, the cell comprises the biomolecule conjugates described herein. In embodiments, the cell comprises biomolecule conjugate of Formula (III), including embodiments thereof. In embodiments, the cell comprises biomolecule conjugate of Formula (VI), including embodiments thereof. In embodiments, the cell comprises biomolecule conjugate of Formula (IX), including embodiments thereof. In embodiments, the cell comprises biomolecule conjugate of Formula (X), including embodiments thereof. In embodiments, the cell comprises biomolecule conjugate of Formula (XI), including embodiments thereof.

A cell can be any prokaryotic or eukaryotic cell. In aspects, the cell is prokaryotic. In aspects, the cell is eukaryotic. In aspects, the cell is a bacterial cell, a fungal cell, a plant cell, an archael cell, or an animal cell. In aspects, the animal cell is an insect cell or a mammalian cell. In aspects, the cell is a bacterial cell. In aspects, the cell is a fungal cell. In aspects, the cell is a plant cell. In aspects, the cell is an archael cell. In aspects, the cell is an animal cell. In aspects, the cell is an insect cell. In aspects, the cell is a mammalian cell. In aspects, the cell is a human cell. For example, any of the compositions described herein can be expressed in bacterial cells such as E. coli, insect cells, yeast or mammalian cells (such as Hela cells, Chinese hamster ovary cells (CHO) or COS cells). In aspects, the cell is a premature mammalian cell, i.e., a pluripotent stem cell. In aspects, the cell is derived from other human tissue. Other suitable cells are known to those skilled in the art.

### Pyrrolysyl-tRNA Synthetase

As described herein, an unnatural amino acid (e.g., of Formula (I), Formula (III) and embodiments thereof) may be inserted into or replace a naturally occurring amino acid in a protein (e.g., a CRISPR protein, an RNA chaperone). In order for the unnatural amino acid to be inserted or replace an amino acid in a protein, it must be capable of being incorporated during proteinogenesis. Thus, the unnatural amino acid must be present on a transfer RNA molecule (tRNA) such that it may be used in translation. Loading of amino acids occurs via an aminoacyl-tRNA synthetase, which is an enzyme that facilitates the attachment of appropriate amino acids to tRNA molecules. However, the attachment of unnatural amino acids to tRNA may not necessarily be accomplished by the naturally occurring aminoacyl-tRNA synthetase. Engineered aminoacyl-tRNA synthetases (e.g., mutant pyrrolysyl-tRNA synthetase (PyIRS)) may be useful for attaching unnatural amino acids to tRNA. A PyIRS mutant library was generated. Compared to previously described PyIRS mutant library, the PyIRS mutant library generated herein was constructed using the new small-intelligent mutagenesis approach that allows a greater number of amino acid residues to be mutated simultaneously (e.g., 10 amino acid residues). Mutant pyrrolysyl-tRNA synthetases and methods for making them are described, for example, in US 2021/0002325, WO 2020/072674, and WO 2020/206341, the disclosures of which are incorporated by reference herein in their entirety.

In embodiments, the disclosure provides a pyrrolysyl-tRNA synthetases having at least 85% sequence identity to the amino acid sequence of SEQ ID NO:49. In embodiments, the disclosure provides a pyrrolysyl-tRNA synthetases having at least 90% sequence identity to the amino acid sequence of SEQ ID NO:49. In embodiments, the disclosure provides a pyrrolysyl-tRNA synthetases having at least 95% sequence identity to the amino acid sequence of SEQ ID NO:49. In embodiments, the disclosure provides a pyrrolysyl-tRNA synthetases comprising the amino acid sequence of SEQ ID NO:49. In embodiments, the disclosure provides a pyrrolysyl-tRNA synthetases as set forth in SEQ ID NO:49.

The disclosure provides a mutant pyrrolysyl-tRNA synthetase, including at least 5 amino acid residues substitutions within the substrate-binding site of the mutant pyrrolysyl-tRNA synthetase. In aspects, the mutant pyrrolysyl-tRNA synthetase comprises at least 5 amino acid residues substitutions in the amino acid sequence of SEQ ID NO:56. In aspects, the substrate-binding site includes residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO:56. In aspects, the at least 5 amino acid residues substitutions are a substitution for alanine at position 302, a substitution for asparagine at position 346, a substitution for cysteine at position 348, a substitution for tyrosine at position 384, and a substitution for tryptophan at position 417 as set forth in the amino acid sequence of SEQ ID NO:56. In aspects, the at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO:56.

In embodiments, the mutant pyrrolysyl-tRNA synthetase is encoded by the nucleic acid sequence of SEQ ID NO:57. In aspects, the mutant pyrrolysyl-tRNA synthetase is encoded by a nucleic acid sequence including the sequence of SEQ ID NO:57. In aspects, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO: 2. In aspects, the mutant pyrrolysyl-tRNA synthetase has an amino acid sequence that has at least 80% identity to SEQ ID NO:57. In aspects, the mutant pyrrolysyl-tRNA synthetase has an amino acid sequence that has at least 85% identity to SEQ ID NO:57. In aspects, the mutant pyrrolysyl-tRNA synthetase has an amino acid sequence that has at least 90% identity to SEQ ID NO:57. In aspects, the mutant pyrrolysyl-tRNA synthetase has an amino acid sequence that has at least 95% identity to SEQ ID NO:57.

In embodiments, the mutant pyrrolysyl-tRNA synthetase has the amino acid sequence of SEQ ID NO:58. In aspects, the mutant pyrrolysyl-tRNA synthetase includes an amino acid sequence of SEQ ID NO:58. In aspects, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:58. In aspects, the mutant pyrrolysyl-tRNA synthetase has an amino acid sequence that has at least 80% identity to SEQ ID NO:58. In aspects, the mutant pyrrolysyl-tRNA synthetase has an amino acid sequence that has at least 85% identity to SEQ ID NO:58. In aspects, the mutant pyrrolysyl-tRNA synthetase has an amino acid sequence that has at least 90% identity to SEQ ID NO:58. In aspects, the mutant pyrrolysyl-tRNA synthetase has an amino acid sequence that has at least 95% identity to SEQ ID NO:58.

### Vectors

The compositions (e.g., mutant pyrrolysyl-tRNA synthetase, tRNA^{Pyl}) provided herein may be delivered to cells using methods well known in the art. Thus, in an embodiment is provided a vector including a nucleic acid sequence encoding a mutant pyrrolysyl-tRNA synthetase as described herein, including embodiments thereof. In embodiments, the vector further includes a nucleic acid sequence encoding tRNA^{Pyl}. In embodiments, the vector comprises a nucleic acid sequence encoding a mutant pyrrolysyl-tRNA synthetase as described herein. In embodiments, the vector further includes a nucleic acid sequence encoding tRNA^{Pyl}.

### Methods of Forming a Biomolecule or Biomolecule Conjugate

The compositions provided herein are useful for forming a biomolecule or biomolecule conjugate. In embodiments, the method of forming a biomolecule (e.g., protein) comprises contacting a biomolecule (e.g., RNA-binding protein), a mutant pyrrolysyl-tRNA synthetase, a tRNA^{Pyl}, and a compound of Formula (I) (including embodiments thereof), thereby producing the biomolecule, i.e., a biomolecule comprising the unnatural amino acid of Formula (I) (including embodiments thereof). The biomolecule produced by the method will comprise the unnatural amino acid side chain of Formula (II) (including embodiments thereof). The mutant pyrrolysyl-tRNA synthetase used in the method of producing the biomolecule is any described herein or known in the art. The tRNA^{Pyl} used in the method of producing the biomolecule is any described herein. In embodiments, the reaction is performed in vitro. In embodiments, the reaction is performed in vivo. In embodiments, the reaction is performed in one or more living cells. In embodiments, the reaction is performed in one or more living bacterial cells. In embodiments, the reaction is performed in one or more living mammalian cells.

In embodiments, the method of forming a biomolecule (e.g., protein) comprises contacting a biomolecule (e.g., protein), a mutant pyrrolysyl-tRNA synthetase, a tRNA^{Pyl}, and a compound of Formula (IV) (including embodiments thereof), thereby producing the biomolecule, i.e., a biomolecule comprising the unnatural amino acid of Formula (IV) (including embodiments thereof). The biomolecule produced by the method will comprise the unnatural amino acid side chain of Formula (IV) (including embodiments thereof). The mutant pyrrolysyl-tRNA synthetase used in the method of producing the biomolecule is any described herein or known in the art (e.g., SEQ ID NO:49). The tRNA^{Pyl} used in the method of producing the biomolecule is any described herein. In embodiments, the reaction is performed in vitro. In embodiments, the reaction is performed in vivo. In embodiments, the reaction is performed in one or more living cells. In embodiments, the reaction is performed in one or more living bacterial cells. In embodiments, the reaction is performed in one or more living mammalian cells.

In embodiments, the method of forming a biomolecule (e.g., protein) comprises contacting a biomolecule (e.g., protein), a mutant pyrrolysyl-tRNA synthetase, a tRNA^{Pyl}, and a compound of Formula (VII) (including embodiments thereof), thereby producing the biomolecule, i.e., a biomolecule comprising the unnatural amino acid of Formula (VIII) (including embodiments thereof). The biomolecule produced by the method will comprise the unnatural amino acid side chain of Formula (VIII) (including embodiments thereof). The mutant pyrrolysyl-tRNA synthetase used in the method of producing the biomolecule is any described herein or known in the art (e.g., SEQ ID NO:56, SEQ ID NO:58). The tRNA^{Pyl} used in the method of producing the biomolecule is any described herein (e.g., SEQ ID NO:59). In embodiments, the reaction is performed in vitro. In embodiments, the reaction is performed in vivo. In embodiments, the reaction is performed in one or more living cells. In embodiments, the reaction is performed in one or more living bacterial cells. In embodiments, the reaction is performed in one or more living mammalian cells.

### Methods of Treatment

The disclosure provides methods of treating or preventing a coronavirus infection in a subject in need thereof by administering to the subject an effective amount of the single-domain antibodies described herein, including embodiments and aspects thereof. The disclosure provides methods of treating or preventing a SARS-CoV infection in a subject in need thereof by administering to the subject an effective amount of the single-domain antibodies described herein, including embodiments and aspects thereof. The disclosure provides methods of treating or preventing a SARS-CoV-1 infection in a subject in need thereof by administering to the subject an effective amount of the single-domain antibodies described herein, including embodiments and aspects thereof. The disclosure provides methods of treating or preventing a SARS-CoV-2 infection in a subject in need thereof by administering to the subject an effective amount of the single-domain antibodies described herein, including embodiments and aspects thereof. The disclosure provides methods of treating or preventing a MERS-CoV infection in a subject in need thereof by administering to the subject an effective amount of the single-domain antibodies described herein, including embodiments and aspects thereof. The disclosure provides methods of treating or preventing COVID-19 in a subject in need thereof by administering to the subject an effective amount of the single-domain antibodies described herein, including embodiments and aspects thereof. The disclosure provides methods of treating or preventing MERS in a subject in need thereof by administering to the subject an effective amount of the single-domain antibodies described herein, including embodiments and aspects thereof. In aspects, the methods are for treating a coronavirus infection, a SARS-CoV infection, a SARS-CoV-2 infection, a SARS-CoV-1 infection, a MERS-CoV infection, COVID-19, or MERS. In aspects, the methods are for preventing a coronavirus infection, a SARS-CoV infection, a SARS-CoV-2 infection, a SARS-CoV-1 infection, a MERS-CoV infection, COVID-19, or MERS. The recombinant proteins can be administered in the form of the pharmaceutical compositions (e.g., vaccines) described herein. In aspects, the subject can be administered another therapeutic agent useful in treating a coronavirus, such as anti-viral agents. In aspects, the recombinant protein is parenterally administered to the subject. In aspects, the recombinant protein is intravenously administered to the subject. In aspects, the recombinant protein is administered to the subject by intravenous infusion. In aspects, the recombinant protein is subcutaneously administered to the subject. In aspects, the recombinant protein is orally administered to the subject. In aspects, the recombinant protein is administered to the subject via nasal inhalation. In aspects, the recombinant protein is administered to the subject via oral inhalation.

In embodiments, the methods of treating a coronavirus infection, a SARS-CoV infection, a SARS-CoV-2 infection, a SARS-CoV-1 infection, a MERS-CoV infection, COVID-19, or MERS comprise administering an effective amount of a single-domain antibody described herein and a second therapeutic agent, such as an antiviral agent. In embodiments, the methods comprise treating a coronavirus infection, a SARS-CoV infection, a SARS-CoV-2 infection, a SARS-CoV-1 infection, a MERS-CoV infection, COVID-19, or MERS by administering to a subject an effective amount of a single-domain antibody described herein and a therapeutic agent selected from the group consisting of remdesivir, dexamethasone, convalescent plasma, bamlanivimab, etesevimab, casiribimab, imdevimab, and a combination of two or more thereof. In embodiments, the methods comprise treating COVID-19 by administering to a subject an effective amount of a single-domain antibody described herein and a therapeutic agent selected from the group consisting of remdesivir, dexamethasone, convalescent plasma, bamlanivimab, etesevimab, casiribimab, imdevimab, and a combination of two or more thereof. In embodiments, the methods comprise administering an effective amount of a single-domain antibody described herein and remdesivir. In embodiments, the methods comprise administering an effective amount of a single-domain antibody described herein and dexamethasone. In embodiments, the methods comprise administering an effective amount of a single-domain antibody described herein and bamlanivimab. In embodiments, the methods comprise administering an effective amount of a single-domain antibody described herein and convalescent plasma. In embodiments, the methods comprise administering an effective amount of a single-domain antibody described herein, bamlanivimab, and etesevimab. In embodiments, the methods comprise administering an effective amount of a single-domain antibody described herein, casirivimab, and imdevimab.

The disclosure provides methods of treating or preventing a coronavirus infection in a subject in need thereof by administering to the subject an effective amount of the recombinant proteins described herein, including embodiments and aspects thereof. The disclosure provides methods of treating or preventing a SARS-CoV infection in a subject in need thereof by administering to the subject an effective amount of the recombinant proteins described herein, including embodiments and aspects thereof. The disclosure provides methods of treating or preventing a SARS-CoV-1 infection in a subject in need thereof by administering to the subject an effective amount of the recombinant proteins described herein, including embodiments and aspects thereof. The disclosure provides methods of treating or preventing a SARS-CoV-2 infection in a subject in need thereof by administering to the subject an effective amount of the recombinant proteins described herein, including embodiments and aspects thereof. The disclosure provides methods of treating or preventing a MERS-CoV infection in a subject in need thereof by administering to the subject an effective amount of the recombinant proteins described herein, including embodiments and aspects thereof. The disclosure provides methods of treating or preventing COVID-19 in a subject in need thereof by administering to the subject an effective amount of the recombinant proteins described herein, including embodiments and aspects thereof. The disclosure provides methods of treating or preventing MERS in a subject in need thereof by administering to the subject an effective amount of the recombinant proteins described herein, including embodiments and aspects thereof. In aspects, the methods are for treating a coronavirus infection, a SARS-CoV infection, a SARS-CoV-2 infection, a SARS-CoV-1 infection, a MERS-CoV infection, COVID-19, or MERS. In aspects, the methods are for preventing a coronavirus infection, a SARS-CoV infection, a SARS-CoV-2 infection, a SARS-CoV-1 infection, a MERS-CoV infection, COVID-19, or MERS. The recombinant proteins can be administered in the form of the pharmaceutical compositions (e.g., vaccines) described herein. In aspects, the subject can be administered another therapeutic agent useful in treating a coronavirus, such as anti-viral agents. In aspects, the recombinant protein is parenterally administered to the subject. In aspects, the recombinant protein is intravenously administered to the subject. In aspects, the recombinant protein is administered to the subject by intravenous infusion. In aspects, the recombinant protein is subcutaneously administered to the subject. In aspects, the recombinant protein is orally administered to the subject. In aspects, the recombinant protein is administered to the subject via nasal inhalation. In aspects, the recombinant protein is administered to the subject via oral inhalation.

In embodiments, the methods of treating a coronavirus infection, a SARS-CoV infection, a SARS-CoV-2 infection, a SARS-CoV-1 infection, a MERS-CoV infection, COVID-19, or MERS comprise administering an effective amount of a recombinant protein described herein and a second therapeutic agent, such as an antiviral agent. In embodiments, the methods comprise treating a coronavirus infection, a SARS-CoV infection, a SARS-CoV-2 infection, a SARS-CoV-1 infection, a MERS-CoV infection, COVID-19, or MERS by administering to a subject an effective amount of a recombinant protein described herein and a therapeutic agent selected from the group consisting of remdesivir, dexamethasone, convalescent plasma, bamlanivimab, etesevimab, casiribimab, imdevimab, and a combination of two or more thereof. In embodiments, the methods comprise treating COVID-19 by administering to a subject an effective amount of a recombinant protein described herein and a therapeutic agent selected from the group consisting of remdesivir, dexamethasone, convalescent plasma, bamlanivimab, etesevimab, casiribimab, imdevimab, and a combination of two or more thereof. In embodiments, the therapeutic agent is remdesivir. In embodiments, the therapeutic agent is dexamethasone. In embodiments, the therapeutic agent is bamlanivimab. In embodiments, the therapeutic agent is convalescent plasma. In embodiments, the therapeutic agent is a combination of bamlanivimab and etesevimab. In embodiments, the therapeutic agent is a combination of casirivimab and imdevimab.

### Imaging and Diagnostic Methods

Provided herein are methods of detecting cancer in a patient in need thereof comprising administering to the patient an effective amount of a nanobody comprising a detectable label as described herein (including all embodiments thereof), thereby detecting cancer in the patient. The method of detecting cancer can comprise identifying the presence of the cancer, the size of the cancer, or the location of the cancer within the body.

Provided herein are methods of monitoring cancer progression or cancer treatment in a patient in need thereof comprising administering to the patient an effective amount of a nanobody comprising a detectable label as described herein (including all embodiments thereof) at a first time point, thereby detecting cancer in the patient; and administering to the patient an effective amount of a nanobody comprising a detectable label as described herein (including all embodiments thereof) at a second time point later than the first time point, thereby monitoring the cancer progression or cancer treatment. In embodiments, the first time point is at the time of diagnosis and prior to the patient receiving a cancer treatment, and the second time point is after the patient has received cancer treatment, such that the effectiveness of the cancer treatment can be determined by the difference in the cancer at the first time point and at the second time point. The difference in the cancer can be, for example, the size of the tumor or metastis. In embodiments, the first time point is after cancer treatment has started and the second time point is after further cancer treatments have been administered, such that the such that the effectiveness of the cancer treatment can be determined by the difference in the cancer at the first time point and at the second time point. In embodiments, the first time point can be when the patient has completed cancer treatment or when the patient is in remission, and the second time point is later than the first time point, such that continued remission or relapse can be identified based on the absence or presence of cancer from the first time point to the second time point.

In embodiments of the methods described herein the cancer expresses HER2 or wherein the cancer overexpresses HER2 relative to a control. In embodiments of the methods described herein the cancer overexpresses HER2 relative to a control.

In embodiments of the methods described herein, any nanobody comprising an unnatural amino acid (e.g., FSY, mFSY, FFY, mFSK, FSK) and a detectable label can be used, including the nanobodies described herein (and embodiments thereof). In embodiments, the nanobody comprises CDR1 as set forth in SEQ ID NO:67, CDR2 as set forth in SEQ ID NO:70, and CDR3 as set forth in SEQ ID NO:69, and a detectable label. The unnatural amino acid on the nanobody binds to the receptor on the cancer cell (e.g., HER2), such that imaging can then be accomplished based on the accumulation of the nanobody, and thereby the detectable agent, at the site of the cancer. In embodiments, the nanobody comprises CDR1 as set forth in SEQ ID NO:67, CDR2 as set forth in SEQ ID NO:68, and CDR3 as set forth in SEQ ID NO:71, and a detectable label.

In embodiments, the detectable label is a detectable label that can be used in medical imaging. In embodiments, the detectable label is a label that can be used for radiography, magnetic resonance imaging, nuclear medicine, ultrasound elastography, photoacoustic imaging, tomography, echocardiography, functional near-infrared spectroscopy, magnetic particle imaging. In embodiments, the detectable label is a label that can be use for tomography. In embodiments, the detectable label is a label that can be used for positron emission tomography.

In embodiments, the detectable label is a radioisotope. In embodiments, the detectable label is an iodine radioisotope. In embodiments, the radioisotope is ¹²³I, ¹²⁴I, ¹²⁵I, or ¹³¹I. In embodiments, the radioisotope is ¹²³I. In embodiments, the radioisotope is ¹²⁴I. In embodiments, the radioisotope is ¹²⁵I. In embodiments, the radioisotope is ¹³¹I. In embodiments, the radioisotope is a positron-emitting radioisotope. In embodiments, the positron-emitting radioisotope is ¹¹C, ¹³N, ¹⁵O, ¹⁸F, ⁶⁴Cu, ⁶⁸Ga, ⁷⁸Br, ⁸²Rb, ⁸⁶Y ⁸⁹Zr, ⁹⁰Y, ²²Na, ²⁶Al, ⁴⁰K, ¹³Sr, or ¹²⁴I. In embodiments, the positron-emitting radioisotope is ¹¹C. In embodiments, the positron-emitting radioisotope is ¹³N. In embodiments, the positron-emitting radioisotope is ¹⁵O. In embodiments, the positron-emitting radioisotope is ¹⁸F. In embodiments, the positron-emitting radioisotope is ⁶⁴Cu. In embodiments, the positron-emitting radioisotope is ¹⁶⁸Ga. In embodiments, the positron-emitting radioisotope is ⁷⁸Br. In embodiments, the positron-emitting radioisotope is ⁸²Rb. In embodiments, the positron-emitting radioisotope is ⁸⁶Y. In embodiments, the positron-emitting radioisotope is ⁸⁹Zr. In embodiments, the positron-emitting radioisotope is ⁹¹Y. In embodiments, the positron-emitting radioisotope is ²²Na. In embodiments, the positron-emitting radioisotope is ²⁶Al. In embodiments, the positron-emitting radioisotope is ⁴⁰K. In embodiments, the positron-emitting radioisotope is ⁸³Sr. In embodiments, the positron-emitting radioisotope is ¹²⁴I. In embodiments, the radioisotope is an alpha-emitting radioisotope. In embodiments, the alpha-emitting radioisotope is ²¹¹At, ²²⁷Th, ²²⁵Ac, ²²³Ra, ²¹³Bi, or ²¹²Bi. In embodiments, the alpha-emitting radioisotope is ²¹¹At. In embodiments, the alpha-emitting radioisotope is ²²⁷Th. In embodiments, the alpha-emitting radioisotope is ²²⁵Ac. In embodiments, the alpha-emitting radioisotope is ²²³Ra. In embodiments, the alpha-emitting radioisotope is ²¹³Bi. In embodiments, the alpha-emitting radioisotope is ²¹²Bi.

### Pharmaceutical Compositions

Any of the proteins (e.g., recombinant proteins and single-domain antibodies) described herein may be administered to a subject in a pharmaceutical composition further comprising a pharmaceutically acceptable excipient. The compositions are suitable for formulation and administration in vitro or in vivo. Suitable carriers and excipients and their formulations are known in the art and described, e.g., Remington: The Science and Practice of Pharmacy, 21st Ed, Lippicott Williams & Wilkins (2005).

The term “pharmaceutical compositon” encompasses compositions administered to a patient for therapeutic purposes (e.g., treating a disease) and/or diagnostic purposes (e.g., medical imaging). Medical imagining includes, without limitation, radiography, magnetic resonance imaging, nuclear medicine, ultrasound elastography, photoacoustic imaging, tomography (e.g., positron emission tomography), echocardiography, functional near-infrared spectroscopy, magnetic particle imaging, and the like.

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful. Pharmaceutically acceptable excipients can be used in pharmaceutical compositions for therapeutic purposes (e.g., treating a disease) and/or diagnostic purposes (e.g., imaging, such as positron emission tomography).

Solutions of the pharmaceutical compositions can be prepared in water suitably mixed with a lipid or surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms. Solutions can be administered, e.g., parenterally, such as subcutaneously or intravenously (e.g., infusion or bolus).

Pharmaceutical compositions can be delivered via intranasal or inhalable solutions. The intranasal composition can be a spray, aerosol, or inhalant. The inhalable composition can be a spray, aerosol, or inhalant. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known in the art.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In aspects, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may be between about 1 to about 75% of the weight of the unit. The amount of nucleic acids in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the recombinant proteins in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for rapid penetration, delivering high concentrations of the active agents to a small area.

For vaccination or immunization purposes the recombinant proteins or single-domain antibodies provided herein including embodiments and aspects thereof, may be formulated and introduced as a vaccine through oral, intradermal, intramuscular, intraperitoneal, intravenous, subcutaneous, intranasal, and via scarification (scratching through the top layers of skin, e.g., using a bifurcated needle) or any other standard route of immunization. Vaccine formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient or any other oral composition as listed above. Alternatively, the vaccines may be administered parenterally as injections (intravenous, intramuscular or subcutaneous). The amount of recombinant proteins used in a vaccine can depend upon a variety of factors including the route of administration, species, and use of booster administration. However, a person of ordinary skill in the art would immediately recognize appropriate and/or equivalent doses looking at dosages of approved whopping cough vaccines for guidance.

The term “adjuvant” refers to a compound that when administered in conjunction with the recombinant proteins provided herein including embodiments thereof augments the immune response to the antigen, but when administered alone does not generate an immune response to the antigen. As described above the recombinant proteins provided herein including embodiments thereof may be used as an adjuvant. Therefore, the term “adjuvant” refers to a compound that when administered in conjunction with a vaccine augments the immune response to the antigen, but when administered alone does not generate an immune response to the antigen. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the recombinant proteins provided herein to elicit an immune response. Adjuvants augment the intrinsic response to an immunogen without causing conformational changes in the immunogen that affect the qualitative form of the response. Exemplary adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, N Y, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other examples of adjuvants are aluminum salts (alum), such as alum hydroxide, alum phosphate, alum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton MA), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi ImmunoChem, Hamilton, MT) containing 2% squalene, 0.2% Tween 80, and one or more bacterial cell wall components from the group consisting of monophosphoryllipid A (MPL), trehalose dimycolate (TDM), and cell wall skeleton (CWS), preferably MPL+CWS (Detox™).

Other adjuvants are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

An adjuvant can be administered with an immunogen as a single composition, or can be administered before, concurrent with or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

### Dose and Dosing Regimens

The dosage and frequency (single or multiple doses) of the proteins (e.g., recombinant proteins, antibodies, antibody variants, single-domain antibodies) administered to a subject can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and proteins (e.g., recombinant proteins, antibodies, antibody variants, single-domain antibodies) described herein. Adjustment and manipulation of established dosages (e.g., frequency and duration) are within the ability of the skilled artisan.

For any composition, proteins (e.g., recombinant proteins, antibodies, antibody variants, single-domain antibodies) described herein, the effective amount can be initially determined from cell culture assays. Target concentrations will be those concentrations of proteins (e.g., recombinant proteins, antibodies, antibody variants, single-domain antibodies) that are capable of achieving the methods described herein, as measured using the methods described herein or known in the art. As is known in the art, effective amounts of proteins (e.g., recombinant proteins, antibodies, antibody variants, single-domain antibodies) for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the proteins (e.g., recombinant proteins, antibodies, antibody variants, single-domain antibodies) may be varied depending upon the requirements of the patient, and whether the purpose is therapeutic or medical imaging. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Dosage amounts and intervals can be adjusted individually to provide levels of the recombinant proteins effective for the particular clinical indication being treated. This will provide a therapeutic regimen that is commensurate with the severity of the individual's disease state.

Utilizing the teachings provided herein, an effective prophylactic, diagnostic, or therapeutic treatment regimen can be planned that does not cause substantial toxicity and yet is effective to treat the clinical disease or symptoms demonstrated by the particular patient. This planning should involve the careful choice of proteins (e.g., recombinant proteins, antibodies, antibody variants, single-domain antibodies) by considering factors such as compound potency, relative bioavailability, patient body weight, presence and severity of adverse side effects.

In embodiments, the proteins (e.g., recombinant proteins, antibodies, antibody variants, single-domain antibodies) are administered to a patient at an amount of about 0.001 mg/kg to about 500 mg/kg. In aspects, the proteins (e.g., recombinant proteins, antibodies, antibody variants, single-domain antibodies) are administered to a patient in an amount of about 0.01 mg/kg, 0.1 mg/kg, 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, or 300 mg/kg. It is understood that where the amount is referred to as “mg/kg,” the amount is milligram per kilogram body weight of the subject being administered with the proteins (e.g., recombinant proteins, antibodies, antibody variants, single-domain antibodies). In aspects, the proteins (e.g., recombinant proteins, antibodies, antibody variants, single-domain antibodies) are administered to a patient in an amount from about 0.01 mg to about 500 mg per day.

### Informal Sequence Listing

In the sequences herein, X^{FSY} refers to the unnatural amino acid FSY; X^{mFSY} refers to the unnatural amino acid metaFSY, and X^{FFY} refers to the unnatural amino acid FFY.

SEQ ID NO: 1 ACE2 Receptor Protein (1-740 aa)
MSSSSWLLLS LVAVTAAQST IEEQAKTELD KENHEAEDLF YQSSLASWNY
NTNITEENVQ NMNNAGDKWS AFLKEQSTLA QMYPLQEIQN LTVKLQLQAL
QQNGSSVLSE DKSKRLNTIL NTMSTIYSTG KVCNPDNPQE CLLLEPGLNE
IMANSLDYNE RLWAWESWRS EVGKQLRPLY EEYVVLKNEM ARANHYEDYG
DYWRGDYEVN GVDGYDYSRG QLIEDVEHTF EEIKPLYEHL HAYVRAKLMN
AYPSYISPIG CLPAHLLGDM WGRFWTNLYS LTVPFGQKPN IDVTDAMVDQ
AWDAQRIFKE AEKFFVSVGL PNMTQGFWEN SMLTDPGNVQ KAVCHPTAWD
LGKGDFRILM CTKVTMDDFL TAHHEMGHIQ YDMAYAAQPF LLRNGANEGF
HEAVGEIMSL SAATPKHLKS IGLLSPDFQE DNETEINFLL KQALTIVGTL
PFTYMLEKWR WMVFKGEIPK DQWMKKWWEM KREIVGVVEP VPHDETYCDP
ASLFHVSNDY SFIRYYTRTL YQFQFQEALC QAAKHEGPLH KCDISNSTEA
GQKLFNMLRL GKSEPWTLAL ENVVGAKNMN VRPLLNYFEP LFTWLKDQNK
NSFVGWSTDW SPYADQSIKV RISLKSALGD KAYEWNDNEM YLFRSSVAYA
MRQYFLKVKN QMILFGEEDV RVANLKPRIS FNFFVTAPKN VSDIIPRTEV
EKAIRMSRSR INDAFRLNDN SLEFLGIQPT LGPPNQPPVS
SEQ ID NO: 2 ACE2 Protein, wherein U at position 34 is FSY
MSSSSWLLLS LVAVTAAQST IEEQAKTELD KFNUEAEDLF YQSSLASWNY
NTNITEENVQ NMNNAGDKWS AFLKEQSTLA QMYPLQEIQN LTVKLQLQAL
QQNGSSVLSE DKSKRLNTIL NTMSTIYSTG KVCNPDNPQE CLLLEPGLNE
IMANSLDYNE RLWAWESWRS EVGKQLRPLY EEYVVLKNEM ARANHYEDYG
DYWRGDYEVN GVDGYDYSRG QLIEDVEHTF EEIKPLYEHL HAYVRAKLMN
AYPSYISPIG CLPAHLLGDM WGRFWTNLYS LTVPFGQKPN IDVTDAMVDQ
AWDAQRIFKE AEKFFVSVGL PNMTQGFWEN SMLTDPGNVQ KAVCHPTAWD
LGKGDFRILM CTKVTMDDFL TAHHEMGHIQ YDMAYAAQPF LLRNGANEGF
HEAVGEIMSL SAATPKHLKS IGLLSPDFQE DNETEINFLL KQALTIVGTL
PFTYMLEKWR WMVFKGEIPK DQWMKKWWEM KREIVGVVEP VPHDETYCDP
ASLFHVSNDY SFIRYYTRTL YQFQFQEALC QAAKHEGPLH KCDISNSTEA
GQKLFNMLRL GKSEPWTLAL ENVVGAKNMN VRPLLNYFEP LFTWLKDQNK
NSFVGWSTDW SPYADQSIKV RISLKSALGD KAYEWNDNEM YLFRSSVAYA
MRQYFLKVKN QMILFGEEDV RVANLKPRIS FNFFVTAPKN VSDIIPRTEV
EKAIRMSRSR INDAFRLNDN SLEFLGIQPT LGPPNQPPVS
SEQ ID NO: 3 ACE2 Protein, where U at position 37 is FSY
MSSSSWLLLS LVAVTAAQST IEEQAKTFLD KFNHEAUDLF YQSSLASWNY
NTNITEENVQ NMNNAGDKWS AFLKEQSTLA QMYPLQEIQN LTVKLQLQAL
QQNGSSVLSE DKSKRLNTIL NTMSTIYSTG KVCNPDNPQE CLLLEPGLNE
IMANSLDYNE RLWAWESWRS EVGKQLRPLY EEYVVLKNEM ARANHYEDYG
DYWRGDYEVN GVDGYDYSRG QLIEDVEHTF EEIKPLYEHL HAYVRAKLMN
AYPSYISPIG CLPAHLLGDM WGRFWTNLYS LTVPFGQKPN IDVTDAMVDQ
AWDAQRIFKE AEKFFVSVGL PNMTQGFWEN SMLTDPGNVQ KAVCHPTAWD
LGKGDFRILM CTKVTMDDFL TAHHEMGHIQ YDMAYAAQPF LLRNGANEGF
HEAVGEIMSL SAATPKHLKS IGLLSPDFQE DNETEINFLL KQALTIVGTL
PFTYMLEKWR WMVFKGEIPK DQWMKKWWEM KREIVGVVEP VPHDETYCDP
ASLFHVSNDY SFIRYYTRTL YQFQFQEALC QAAKHEGPLH KCDISNSTEA
GQKLFNMLRL GKSEPWTLAL ENVVGAKNMN VRPLLNYFEP LFTWLKDQNK
NSFVGWSTDW SPYADQSIKV RISLKSALGD KAYEWNDNEM YLFRSSVAYA
MRQYFLKVKN QMILFGEEDV RVANLKPRIS FNFFVTAPKN VSDIIPRTEV
EKAIRMSRSR INDAFRLNDN SLEFLGIQPT LGPPNQPPVS
SEQ ID NO: 4 ACE2 Protein, where U at position 42 is FSY
MSSSSWLLLS LVAVTAAQST IEEQAKTFLD KFNHEAEDLF YUSSLASWNY
NTNITEENVQ NMNNAGDKWS AFLKEQSTLA QMYPLQEIQN LTVKLQLQAL
QQNGSSVLSE DKSKRLNTIL NTMSTIYSTG KVCNPDNPQE CLLLEPGLNE
IMANSLDYNE RLWAWESWRS EVGKQLRPLY EEYVVLKNEM ARANHYEDYG
DYWRGDYEVN GVDGYDYSRG QLIEDVEHTF EEIKPLYEHL HAYVRAKLMN
AYPSYISPIG CLPAHLLGDM WGRFWTNLYS LTVPFGQKPN IDVTDAMVDQ
AWDAQRIFKE AEKFFVSVGL PNMTQGFWEN SMLTDPGNVQ KAVCHPTAWD
LGKGDFRILM CTKVTMDDFL TAHHEMGHIQ YDMAYAAQPF LLRNGANEGF
HEAVGEIMSL SAATPKHLKS IGLLSPDFQE DNETEINFLL KQALTIVGTL
PFTYMLEKWR WMVFKGEIPK DQWMKKWWEM KREIVGVVEP VPHDETYCDP
ASLFHVSNDY SFIRYYTRTL YQFQFQEALC QAAKHEGPLH KCDISNSTEA
GQKLFNMLRL GKSEPWTLAL ENVVGAKNMN VRPLLNYFEP LFTWLKDQNK
NSFVGWSTDW SPYADQSIKV RISLKSALGD KAYEWNDNEM YLFRSSVAYA
MRQYFLKVKN QMILFGEEDV RVANLKPRIS FNFFVTAPKN VSDIIPRTEV
EKAIRMSRSR INDAFRINDN SLEFLGIQPT LGPPNQPPVS
SEQ ID NO: 5
SARS-COV-2 Spike (S) Protein, with the Amino Acid Residues numbered as 319 to 541
RVQPTESIVR FPNITNLCPF GEVFNATRFA SVYAWNRKRI SNCVADYSVL
YNSASFSTFK CYGVSPTKLN DLCFTNVYAD SFVIRGDEVR QIAPGQTGKI
ADYNYKLPDD FTGCVIAWNS NNLDSKVGGN YNYLYRLFRK SNLKPFERDI
STEIYQAGST PCNGVEGFNC YFPLQSYGFQ PTNGVGYQPY RVVVLSFELL
HAPATVCGPK KSTNLVKNKC VNF
SEQ ID NO: 6 Primer for ACE2-For:
5′CTAGCGTTTAAACTTAAGCTTGCCACCATGTCAAGCTCTTCCTGGCTC
SEQ ID NO: 7 Primer for ACE2-Rev:
5′CACACTGGACTAGTGGATCCTTAGTGATGGTGATGATGATGGGAAACAGGGGGCT
GG
SEQ ID NO: 8 ACE2-D30TAG-F
CCAAGACATTTTTGTAGAAGTTTAACCACG
SEQ ID NO: 9 ACE2-D30TAG-R
CTACAAAAATGTCTTGGCCTGTTCCTC
SEQ ID NO: 10 ACE2-D38TAG-F
GTTTAACCACGAAGCCGAATAGCTGTTCTATCAAAG
SEQ ID NO: 11 = ACE2-D38TAG-R = CTATTCGGCTTCGTGGTTAAACTTG
SEQ ID NO: 12 ACE2-E37TAG-F
CAAGTTTAACCACGAAGCCTAGGACCTGTTCTATCAAAG
SEQ ID NO: 13 = ACE2-E37TAG-R = CTAGGCTTCGTGGTTAAACTTGTC
SEQ ID NO: 14 = ACE2-H34TAG-F
CATTTTTGGACAAGTTTAACTAGGAAGCCGAAGACCTG
SEQ ID NO: 15 = ACE2-H34TAG-R =
GGCTTCCTAGTTAAACTTGTCCAAAAATG
SEQ ID NO: 16 = ACE2-Q42TAG-F
CGAAGACCTGTTCTATTAGAGTTCACTTGCTTC
SEQ ID NO: 17 = ACE2-Q42TAG-R =
CTCTAATAGAACAGGTCTTCGGCTTCGTG
SEQ ID NO: 18 ACE2-Y83TAG-F
CACACTTGCCCAAATGTAGCCACTACAAGAAAT
SEQ ID NO: 19 ACE2-Y83TAG-R
GTGGCTACATTTGGGCAAGTGTGGACTG
SEQ ID NO: 20 Gene sequences of ACE2 proteins = ACE2-wt
atgtcaagctcttcctggctccttctcagccttgttgctgtaactgctgctcagtccaccattgaggaacaggccaagacatttttggacaagttt
aaccacgaagccgaagacctgttctatcaaagttcacttgcttcttggaattataacaccaatattactgaagagaatgtccaaaacatgaata
atgctggggacaaatggtctgcctttttaaaggaacagtccacacttgcccaaatgtatccactacaagaaattcagaatctcacagtcaagc
ttcagctgcaggctcttcagcaaaatgggtcttcagtgctctcagaagacaagagcaaacggttgaacacaattctaaatacaatgagcacc
atctacagtactggaaaagtttgtaacccagataatccacaagaatgcttattacttgaaccaggtttgaatgaaataatggcaaacagtttaga
ctacaatgagaggctctgggcttgggaaagctggagatctgaggtcggcaagcagctgaggccattatatgaagagtatgtggtcttgaaa
aatgagatggcaagagcaaatcattatgaggactatggggattattggagaggagactatgaagtaaatggggtagatggctatgactaca
gccgcggccagttgattgaagatgtggaacatacctttgaagagattaaaccattatatgaacatcttcatgcctatgtgagggcaaagttgat
gaatgcctatccttcctatatcagtccaattggatgcctccctgctcatttgcttggtgatatgtggggtagattttggacaaatctgtactctttga
cagttccctttggacagaaaccaaacatagatgttactgatgcaatggtggaccaggcctgggatgcacagagaatattcaaggaggccga
gaagttctttgtatctgttggtcttcctaatatgactcaaggattctgggaaaattccatgctaacggacccaggaaatgttcagaaagcagtct
gccatcccacagcttgggacctggggaagggcgacttcaggatccttatgtgcacaaaggtgacaatggacgacttcctgacagctcatca
tgagatggggcatatccagtatgatatggcatatgctgcacaaccttttctgctaagaaatggagctaatgaaggattccatgaagctgttgg
ggaaatcatgtcactttctgcagccacacctaagcatttaaaatccattggtcttctgtcacccgattttcaagaagacaatgaaacagaaataa
acttcctgctcaaacaagcactcacgattgttgggactctgccatttacttacatgttagagaagtggaggtggatggtctttaaaggggaaat
tcccaaagaccagtggatgaaaaagtggtgggagatgaagcgagagatagttggggggggaacctgtgccccatgatgaaacatact
gtgaccccgcatctctgttccatgtttctaatgattactcattcattcgatattacacaaggaccctttaccaattccagtttcaagaagcactttgt
caagcagctaaacatgaaggccctctgcacaaatgtgacatctcaaactctacagaagctggacagaaactgttcaatatgctgaggcttgg
aaaatcagaaccctggaccctagcattggaaaatgttgtaggagcaaagaacatgaatgtaaggccactgctcaactactttgagcccttatt
tacctggctgaaagaccagaacaagaattcttttgtgggatggagtaccgactggagtccatatgcagaccaaagcatcaaagtgaggata
agcctaaaatcagctcttggagataaagcatatgaatggaacgacaatgaaatgtacctgttccgatcatctgttgcatatgctatgaggcagt
actttttaaaagtaaaaaatcagatgattctttttggggaggaggatgtgcgagtggctaatttgaaaccaagaatctcctttaattttttgtcact
gcacctaaaaatgtgtctgatatcattcctagaactgaagttgaaaaggccatcaggatgtcccggagccgtatcaatgatgctttccgtctga
atgacaacagcctagagtttctggggatacagccaacacttggacctcctaaccagccccctgtttcccatcatcatcaccatcac
SEQ ID NO: 21 ACE2-D30TAG, where the D30TAG is capitalized and underlined
atgtcaagctcttcctggctccttctcagccttgttgctgtaactgctgctcagtccaccattgaggaacaggccaagacatttttgTAGaag
tttaaccacgaagccgaagacctgttctatcaaagttcacttgcttcttggaattataacaccaatattactgaagagaatgtccaaaacatgaa
taatgctggggacaaatggtctgcctttttaaaggaacagtccacacttgcccaaatgtatccactacaagaaattcagaatctcacagtcaa
gcttcagctgcaggctcttcagcaaaatgggtcttcagtgctctcagaagacaagagcaaacggttgaacacaattctaaatacaatgagca
ccatctacagtactggaaaagtttgtaacccagataatccacaagaatgcttattacttgaaccaggtttgaatgaaataatggcaaacagttta
gactacaatgagaggctctgggcttgggaaagctggagatctgaggtcggcaagcagctgaggccattatatgaagagtatgtggtcttga
aaaatgagatggcaagagcaaatcattatgaggactatggggattattggagaggagactatgaagtaaatggggtagatggctatgacta
cagccgcggccagttgattgaagatgtggaacatacctttgaagagattaaaccattatatgaacatcttcatgcctatgtgagggcaaagtt
gatgaatgcctatccttcctatatcagtccaattggatgcctccctgctcatttgcttggtgatatgtggggtagattttggacaaatctgtactctt
tgacagttccctttggacagaaaccaaacatagatgttactgatgcaatggtggaccaggcctgggatgcacagagaatattcaaggaggc
cgagaagttctttgtatctgttggtcttcctaatatgactcaaggattctgggaaaattccatgctaacggacccaggaaatgttcagaaagca
gtctgccatcccacagcttgggacctggggaagggcgacttcaggatccttatgtgcacaaaggtgacaatggacgacttcctgacagctc
atcatgagatggggcatatccagtatgatatggcatatgctgcacaaccttttctgctaagaaatggagctaatgaaggattccatgaagctgt
tggggaaatcatgtcactttctgcagccacacctaagcatttaaaatccattggtcttctgtcacccgattttcaagaagacaatgaaacagaa
ataaacttcctgctcaaacaagcactcacgattgttgggactctgccatttacttacatgttagagaagtggaggtggatggtctttaaagggg
aaattcccaaagaccagtggatgaaaaagtggtgggagatgaagcgagagatagttggggtggtggaacctgtgccccatgatgaaacat
actgtgaccccgcatctctgttccatgtttctaatgattactcattcattcgatattacacaaggaccctttaccaattccagtttcaagaagcactt
tgtcaagcagctaaacatgaaggccctctgcacaaatgtgacatctcaaactctacagaagctggacagaaactgttcaatatgctgaggct
tggaaaatcagaaccctggaccctagcattggaaaatgttgtaggagcaaagaacatgaatgtaaggccactgctcaactactttgagccct
tatttacctggctgaaagaccagaacaagaattcttttgtgggatggagtaccgactggagtccatatgcagaccaaagcatcaaagtgagg
ataagcctaaaatcagctcttggagataaagcatatgaatggaacgacaatgaaatgtacctgttccgatcatctgttgcatatgctatgaggc
agtactttttaaaagtaaaaaatcagatgattctttttggggaggaggatgtgcgagtggctaatttgaaaccaagaatctcctttaatttctttgt
cactgcacctaaaaatgtgtctgatatcattcctagaactgaagttgaaaaggccatcaggatgtcccggagccgtatcaatgatgctttccgt
ctgaatgacaacagcctagagtttctggggatacagccaacacttggacctcctaaccagccccctgtttcccatcatcatcaccatcac
SEQ ID NO: 22 ACE2 Protein, wherein U at position 30 is FSY
MSSSSWLLLS LVAVTAAQST IEEQAKTFLU KFNHEAEDLF YQSSLASWNY
NTNITEENVQ NMNNAGDKWS AFLKEQSTLA QMYPLQEIQN LTVKLQLQAL
QQNGSSVLSE DKSKRLNTIL NTMSTIYSTG KVCNPDNPQE CLLLEPGLNE
IMANSLDYNE RLWAWESWRS EVGKQLRPLY EEYVVLKNEM ARANHYEDYG
DYWRGDYEVN GVDGYDYSRG QLIEDVEHTF EEIKPLYEHL HAYVRAKLMN
AYPSYISPIG CLPAHLLGDM WGRFWTNLYS LTVPFGQKPN IDVTDAMVDQ
AWDAQRIFKE AEKFFVSVGL PNMTQGFWEN SMLTDPGNVQ KAVCHPTAWD
LGKGDFRILM CTKVTMDDFL TAHHEMGHIQ YDMAYAAQPF LLRNGANEGF
HEAVGEIMSL SAATPKHLKS IGLLSPDFQE DNETEINFLL KQALTIVGTL
PFTYMLEKWR WMVFKGEIPK DQWMKKWWEM KREIVGVVEP VPHDETYCDP
ASLFHVSNDY SFIRYYTRTL YQFQFQEALC QAAKHEGPLH KCDISNSTEA
GQKLFNMLRL GKSEPWTLAL ENVVGAKNMN VRPLLNYFEP LFTWLKDQNK
NSFVGWSTDW SPYADQSIKV RISLKSALGD KAYEWNDNEM YLFRSSVAYA
MRQYFLKVKN QMILFGEEDV RVANLKPRIS FNFFVTAPKN VSDIIPRTEV
EKAIRMSRSR INDAFRLNDN SLEFLGIQPT LGPPNQPPVS
SEQ ID NO: 23 ACE2-H34TAG, where the H34TAG is capatilized and underlined
atgtcaagctcttcctggctccttctcagccttgttgctgtaactgctgctcagtccaccattgaggaacaggccaagacatttttggacaagttt
aacTAGgaagccgaagacctgttctatcaaagttcacttgcttcttggaattataacaccaatattactgaagagaatgtccaaaacatgaa
taatgctggggacaaatggtctgcctttttaaaggaacagtccacacttgcccaaatgtatccactacaagaaattcagaatctcacagtcaa
gcttcagctgcaggctcttcagcaaaatgggtcttcagtgctctcagaagacaagagcaaacggttgaacacaattctaaatacaatgagca
ccatctacagtactggaaaagtttgtaacccagataatccacaagaatgcttattacttgaaccaggtttgaatgaaataatggcaaacagttta
gactacaatgagaggctctgggcttgggaaagctggagatctgaggtcggcaagcagctgaggccattatatgaagagtatgtggtcttga
aaaatgagatggcaagagcaaatcattatgaggactatggggattattggagaggagactatgaagtaaatggggtagatggctatgacta
cagccgcggccagttgattgaagatgtggaacatacctttgaagagattaaaccattatatgaacatcttcatgcctatgtgagggcaaagtt
gatgaatgcctatccttcctatatcagtccaattggatgcctccctgctcatttgcttggtgatatgtggggtagattttggacaaatctgtactctt
tgacagttccctttggacagaaaccaaacatagatgttactgatgcaatggtggaccaggcctgggatgcacagagaatattcaaggaggc
cgagaagttctttgtatctgttggtcttcctaatatgactcaaggattctgggaaaattccatgctaacggacccaggaaatgttcagaaagca
gtctgccatcccacagcttgggacctggggaagggcgacttcaggatccttatgtgcacaaaggtgacaatggacgacttcctgacagctc
atcatgagatggggcatatccagtatgatatggcatatgctgcacaaccttttctgctaagaaatggagctaatgaaggattccatgaagctgt
tggggaaatcatgtcactttctgcagccacacctaagcatttaaaatccattggtcttctgtcacccgattttcaagaagacaatgaaacagaa
ataaacttcctgctcaaacaagcactcacgattgttgggactctgccatttacttacatgttagagaagtggaggtggatggtctttaaagggg
aaattcccaaagaccagtggatgaaaaagtggtgggagatgaagcgagagatagttggggggggaacctgtgccccatgatgaaacat
actgtgaccccgcatctctgttccatgtttctaatgattactcattcattcgatattacacaaggaccctttaccaattccagtttcaagaagcactt
tgtcaagcagctaaacatgaaggccctctgcacaaatgtgacatctcaaactctacagaagctggacagaaactgttcaatatgctgaggct
tggaaaatcagaaccctggaccctagcattggaaaatgttgtaggagcaaagaacatgaatgtaaggccactgctcaactactttgagccct
tatttacctggctgaaagaccagaacaagaattcttttgtgggatggagtaccgactggagtccatatgcagaccaaagcatcaaagtgagg
ataagcctaaaatcagctcttggagataaagcatatgaatggaacgacaatgaaatgtacctgttccgatcatctgttgcatatgctatgaggc
agtactttttaaaagtaaaaaatcagatgattctttttggggaggaggatgtgcgagtggctaatttgaaaccaagaatctcctttaatttctttgt
cactgcacctaaaaatgtgtctgatatcattcctagaactgaagttgaaaaggccatcaggatgtcccggagccgtatcaatgatgctttccgt
ctgaatgacaacagcctagagtttctggggatacagccaacacttggacctcctaaccagccccctgtttcccatcatcatcaccatcac
SEQ ID NO: 24 ACE2-E37TAG, where the E37TAG is capatilized and underlined
atgtcaagctcttcctggctccttctcagccttgttgctgtaactgctgctcagtccaccattgaggaacaggccaagacatttttggacaagttt
aaccacgaagccTAGgacctgttctatcaaagttcacttgcttcttggaattataacaccaatattactgaagagaatgtccaaaacatgaa
taatgctggggacaaatggtctgcctttttaaaggaacagtccacacttgcccaaatgtatccactacaagaaattcagaatctcacagtcaa
gcttcagctgcaggctcttcagcaaaatgggtcttcagtgctctcagaagacaagagcaaacggttgaacacaattctaaatacaatgagca
ccatctacagtactggaaaagtttgtaacccagataatccacaagaatgcttattacttgaaccaggtttgaatgaaataatggcaaacagttta
gactacaatgagaggctctgggcttgggaaagctggagatctgaggtcggcaagcagctgaggccattatatgaagagtatgtggtcttga
aaaatgagatggcaagagcaaatcattatgaggactatggggattattggagaggagactatgaagtaaatggggtagatggctatgacta
cagccgcggccagttgattgaagatgtggaacatacctttgaagagattaaaccattatatgaacatcttcatgcctatgtgagggcaaagtt
gatgaatgcctatccttcctatatcagtccaattggatgcctccctgctcatttgcttggtgatatgtggggtagattttggacaaatctgtactctt
tgacagttccctttggacagaaaccaaacatagatgttactgatgcaatggtggaccaggcctgggatgcacagagaatattcaaggaggc
cgagaagttctttgtatctgttggtcttcctaatatgactcaaggattctgggaaaattccatgctaacggacccaggaaatgttcagaaagca
gtctgccatcccacagcttgggacctggggaagggcgacttcaggatccttatgtgcacaaaggtgacaatggacgacttcctgacagctc
atcatgagatggggcatatccagtatgatatggcatatgctgcacaaccttttctgctaagaaatggagctaatgaaggattccatgaagctgt
tggggaaatcatgtcactttctgcagccacacctaagcatttaaaatccattggtcttctgtcacccgattttcaagaagacaatgaaacagaa
ataaacttcctgctcaaacaagcactcacgattgttgggactctgccatttacttacatgttagagaagtggaggtggatggtctttaaagggg
aaattcccaaagaccagtggatgaaaaagtggtgggagatgaagcgagagatagttggggtggtggaacctgtgccccatgatgaaacat
actgtgaccccgcatctctgttccatgtttctaatgattactcattcattcgatattacacaaggaccctttaccaattccagtttcaagaagcactt
tgtcaagcagctaaacatgaaggccctctgcacaaatgtgacatctcaaactctacagaagctggacagaaactgttcaatatgctgaggct
tggaaaatcagaaccctggaccctagcattggaaaatgttgtaggagcaaagaacatgaatgtaaggccactgctcaactactttgagccct
tatttacctggctgaaagaccagaacaagaattcttttgtgggatggagtaccgactggagtccatatgcagaccaaagcatcaaagtgagg
ataagcctaaaatcagctcttggagataaagcatatgaatggaacgacaatgaaatgtacctgttccgatcatctgttgcatatgctatgaggc
agtactttttaaaagtaaaaaatcagatgattctttttggggaggaggatgtgcgagtggctaatttgaaaccaagaatctcctttaatttctttgt
cactgcacctaaaaatgtgtctgatatcattcctagaactgaagttgaaaaggccatcaggatgtcccggagccgtatcaatgatgctttccgt
ctgaatgacaacagcctagagtttctggggatacagccaacacttggacctcctaaccagccccctgtttcccatcatcatcaccatcac
SEQ ID NO: 25 ACE2-D38TAG, where the D38TAG is capatilized and underlined
atgtcaagctcttcctggctccttctcagccttgttgctgtaactgctgctcagtccaccattgaggaacaggccaagacatttttggacaagttt
aaccacgaagccgaaTAGctgttctatcaaagttcacttgcttcttggaattataacaccaatattactgaagagaatgtccaaaacatgaa
taatgctggggacaaatggtctgcctttttaaaggaacagtccacacttgcccaaatgtatccactacaagaaattcagaatctcacagtcaa
gcttcagctgcaggctcttcagcaaaatgggtcttcagtgctctcagaagacaagagcaaacggttgaacacaattctaaatacaatgagca
ccatctacagtactggaaaagtttgtaacccagataatccacaagaatgcttattacttgaaccaggtttgaatgaaataatggcaaacagttta
gactacaatgagaggctctgggcttgggaaagctggagatctgaggtcggcaagcagctgaggccattatatgaagagtatgtggtcttga
aaaatgagatggcaagagcaaatcattatgaggactatggggattattggagaggagactatgaagtaaatggggtagatggctatgacta
cagccgcggccagttgattgaagatgtggaacatacctttgaagagattaaaccattatatgaacatcttcatgcctatgtgagggcaaagtt
gatgaatgcctatccttcctatatcagtccaattggatgcctccctgctcatttgcttggtgatatgtggggtagattttggacaaatctgtactctt
tgacagttccctttggacagaaaccaaacatagatgttactgatgcaatggtggaccaggcctgggatgcacagagaatattcaaggaggc
cgagaagttctttgtatctgttggtcttcctaatatgactcaaggattctgggaaaattccatgctaacggacccaggaaatgttcagaaagca
gtctgccatcccacagcttgggacctggggaagggcgacttcaggatccttatgtgcacaaaggtgacaatggacgacttcctgacagctc
atcatgagatggggcatatccagtatgatatggcatatgctgcacaaccttttctgctaagaaatggagctaatgaaggattccatgaagctgt
tggggaaatcatgtcactttctgcagccacacctaagcatttaaaatccattggtcttctgtcacccgattttcaagaagacaatgaaacagaa
ataaacttcctgctcaaacaagcactcacgattgttgggactctgccatttacttacatgttagagaagtggaggtggatggtctttaaagggg
aaattcccaaagaccagtggatgaaaaagtggtgggagatgaagcgagagatagttggggtggtggaacctgtgccccatgatgaaacat
actgtgaccccgcatctctgttccatgtttctaatgattactcattcattcgatattacacaaggaccctttaccaattccagtttcaagaagcactt
tgtcaagcagctaaacatgaaggccctctgcacaaatgtgacatctcaaactctacagaagctggacagaaactgttcaatatgctgaggct
tggaaaatcagaaccctggaccctagcattggaaaatgttgtaggagcaaagaacatgaatgtaaggccactgctcaactactttgagccct
tatttacctggctgaaagaccagaacaagaattcttttgtgggatggagtaccgactggagtccatatgcagaccaaagcatcaaagtgagg
ataagcctaaaatcagctcttggagataaagcatatgaatggaacgacaatgaaatgtacctgttccgatcatctgttgcatatgctatgaggc
agtactttttaaaagtaaaaaatcagatgattctttttggggaggaggatgtgcgagtggctaatttgaaaccaagaatctcctttaatttctttgt
cactgcacctaaaaatgtgtctgatatcattcctagaactgaagttgaaaaggccatcaggatgtcccggagccgtatcaatgatgctttccgt
ctgaatgacaacagcctagagtttctggggatacagccaacacttggacctcctaaccagccccctgtttcccatcatcatcaccatcac
SEQ ID NO: 26 ACE2 Protein, wherein U at position 38 is FSY
MSSSSWLLLS LVAVTAAQST IEEQAKTFLD KfNHEAEULF YQSSLASWNY
NTNITEENVQ NMNNAGDKWS AFLKEQSTLA QMYPLQEIQN LTVKLQLQAL
QQNGSSVLSE DKSKRLNTIL NTMSTIYSTG KVCNPDNPQE CLLLEPGLNE
IMANSLDYNE RLWAWESWRS EVGKQLRPLY EEYVVLKNEM ARANHYEDYG
DYWRGDYEVN GVDGYDYSRG QLIEDVEHTF EEIKPLYEHL HAYVRAKLMN
AYPSYISPIG CLPAHLLGDM WGRFWTNLYS LTVPFGQKPN IDVTDAMVDQ
AWDAQRIFKE AEKFFVSVGL PNMTQGFWEN SMLTDPGNVQ KAVCHPTAWD
LGKGDFRILM CTKVTMDDFL TAHHEMGHIQ YDMAYAAQPF LLRNGANEGF
HEAVGEIMSL SAATPKHLKS IGLLSPDFQE DNETEINFLL KQALTIVGTL
PFTYMLEKWR WMVFKGEIPK DQWMKKWWEM KREIVGVVEP VPHDETYCDP
ASLFHVSNDY SFIRYYTRTL YQFQFQEALC QAAKHEGPLH KCDISNSTEA
GQKLFNMLRL GKSEPWTLAL ENVVGAKNMN VRPLLNYFEP LFTWLKDQNK
NSFVGWSTDW SPYADQSIKV RISLKSALGD KAYEWNDNEM YLFRSSVAYA
MRQYFLKVKN QMILFGEEDV RVANLKPRIS FNFFVTAPKN VSDIIPRTEV
EKAIRMSRSR INDAFRlNDN SLEFLGIQPT LGPPNQPPVS
SEQ ID NO: 27 ACE2-Q42TAG, where the Q42TAG is capatilized and underlined
atgtcaagctcttcctggctccttctcagccttgttgctgtaactgctgctcagtccaccattgaggaacaggccaagacatttttggacaagttt
aaccacgaagccgaagacctgttctatTAGagttcacttgcttcttggaattataacaccaatattactgaagagaatgtccaaaacatgaa
taatgctggggacaaatggtctgcctttttaaaggaacagtccacacttgcccaaatgtatccactacaagaaattcagaatctcacagtcaa
gcttcagctgcaggctcttcagcaaaatgggtcttcagtgctctcagaagacaagagcaaacggttgaacacaattctaaatacaatgagca
ccatctacagtactggaaaagtttgtaacccagataatccacaagaatgcttattacttgaaccaggtttgaatgaaataatggcaaacagttta
gactacaatgagaggctctgggcttgggaaagctggagatctgaggtcggcaagcagctgaggccattatatgaagagtatgtggtcttga
aaaatgagatggcaagagcaaatcattatgaggactatggggattattggagaggagactatgaagtaaatggggtagatggctatgacta
cagccgcggccagttgattgaagatgtggaacatacctttgaagagattaaaccattatatgaacatcttcatgcctatgtgagggcaaagtt
gatgaatgcctatccttcctatatcagtccaattggatgcctccctgctcatttgcttggtgatatgtggggtagattttggacaaatctgtactctt
tgacagttccctttggacagaaaccaaacatagatgttactgatgcaatggtggaccaggcctgggatgcacagagaatattcaaggaggc
cgagaagttctttgtatctgttggtcttcctaatatgactcaaggattctgggaaaattccatgctaacggacccaggaaatgttcagaaagca
gtctgccatcccacagcttgggacctggggaagggcgacttcaggatccttatgtgcacaaaggtgacaatggacgacttcctgacagctc
atcatgagatggggcatatccagtatgatatggcatatgctgcacaaccttttctgctaagaaatggagctaatgaaggattccatgaagctgt
tggggaaatcatgtcactttctgcagccacacctaagcatttaaaatccattggtcttctgtcacccgattttcaagaagacaatgaaacagaa
ataaacttcctgctcaaacaagcactcacgattgttgggactctgccatttacttacatgttagagaagtggaggtggatggtctttaaagggg
aaattcccaaagaccagtggatgaaaaagtggtgggagatgaagcgagagatagttggggtggtggaacctgtgccccatgatgaaacat
actgtgaccccgcatctctgttccatgtttctaatgattactcattcattegatattacacaaggaccctttaccaattccagtttcaagaagcactt
tgtcaagcagctaaacatgaaggccctctgcacaaatgtgacatctcaaactctacagaagctggacagaaactgttcaatatgctgaggct
tggaaaatcagaaccctggaccctagcattggaaaatgttgtaggagcaaagaacatgaatgtaaggccactgctcaactactttgagccct
tatttacctggctgaaagaccagaacaagaattcttttgtgggatggagtaccgactggagtccatatgcagaccaaagcatcaaagtgagg
ataagcctaaaatcagctcttggagataaagcatatgaatggaacgacaatgaaatgtacctgttccgatcatctgttgcatatgctatgaggc
agtactttttaaaagtaaaaaatcagatgattctttttggggaggaggatgtgcgagtggctaatttgaaaccaagaatctcctttaatttctttgt
cactgcacctaaaaatgtgtctgatatcattcctagaactgaagttgaaaaggccatcaggatgtcccggagccgtatcaatgatgctttccgt
ctgaatgacaacagcctagagtttctggggatacagccaacacttggacctcctaaccagccccctgtttcccatcatcatcaccatcac
SEQ ID NO: 28 ACE2-Y83TAG, where the Y83TAG is capatilized and underlined
atgtcaagctcttcctggctccttctcagccttgttgctgtaactgctgctcagtccaccattgaggaacaggccaagacatttttggacaagttt
aaccacgaagccgaagacctgttctatcaaagttcacttgcttcttggaattataacaccaatattactgaagagaatgtccaaaacatgaata
atgctggggacaaatggtctgcctttttaaaggaacagtccacacttgcccaaatgTAGccactacaagaaattcagaatctcacagtcaa
gcttcagctgcaggctcttcagcaaaatgggtcttcagtgctctcagaagacaagagcaaacggttgaacacaattctaaatacaatgagca
ccatctacagtactggaaaagtttgtaacccagataatccacaagaatgcttattacttgaaccaggtttgaatgaaataatggcaaacagttta
gactacaatgagaggctctgggcttgggaaagctggagatctgaggtcggcaagcagctgaggccattatatgaagagtatgtggtcttga
aaaatgagatggcaagagcaaatcattatgaggactatggggattattggagaggagactatgaagtaaatggggtagatggctatgacta
cagccgcggccagttgattgaagatgtggaacatacctttgaagagattaaaccattatatgaacatcttcatgcctatgtgagggcaaagtt
gatgaatgcctatccttcctatatcagtccaattggatgcctccctgctcatttgcttggtgatatgtggggtagattttggacaaatctgtactctt
tgacagttccctttggacagaaaccaaacatagatgttactgatgcaatggtggaccaggcctgggatgcacagagaatattcaaggaggc
cgagaagttctttgtatctgttggtcttcctaatatgactcaaggattctgggaaaattccatgctaacggacccaggaaatgttcagaaagca
gtctgccatcccacagcttgggacctggggaagggcgacttcaggatccttatgtgcacaaaggtgacaatggacgacttcctgacagctc
atcatgagatggggcatatccagtatgatatggcatatgctgcacaaccttttctgctaagaaatggagctaatgaaggattccatgaagctgt
tggggaaatcatgtcactttctgcagccacacctaagcatttaaaatccattggtcttctgtcacccgattttcaagaagacaatgaaacagaa
ataaacttcctgctcaaacaagcactcacgattgttgggactctgccatttacttacatgttagagaagtggaggtggatggtctttaaagggg
aaattcccaaagaccagtggatgaaaaagtggtgggagatgaagcgagagatagttggggtggtggaacctgtgccccatgatgaaacat
actgtgaccccgcatctctgttccatgtttctaatgattactcattcattcgatattacacaaggaccctttaccaattccagtttcaagaagcactt
tgtcaagcagctaaacatgaaggccctctgcacaaatgtgacatctcaaactctacagaagctggacagaaactgttcaatatgctgaggct
tggaaaatcagaaccctggaccctagcattggaaaatgttgtaggagcaaagaacatgaatgtaaggccactgctcaactactttgagccct
tatttacctggctgaaagaccagaacaagaattcttttgtgggatggagtaccgactggagtccatatgcagaccaaagcatcaaagtgagg
ataagcctaaaatcagctcttggagataaagcatatgaatggaacgacaatgaaatgtacctgttccgatcatctgttgcatatgctatgaggc
agtactttttaaaagtaaaaaatcagatgattctttttggggaggaggatgtgcgagtggctaatttgaaaccaagaatctcctttaatttctttgt
cactgcacctaaaaatgtgtctgatatcattcctagaactgaagttgaaaaggccatcaggatgtcccggagccgtatcaatgatgctttccgt
ctgaatgacaacagcctagagtttctggggatacagccaacacttggacctcctaaccagccccctgtttcccatcatcatcaccatcac
SEQ ID NO: 29 ACE2 Protein, wherein U at position 83 is FSY
MSSSSWLLLS LVAVTAAQST IEEQAKTFLD KFNHEAEDLF YQSSLASWNY
NTNITEENVQ NMNNAGDKWS AFLKEQSTLA QMUPLQEIQN LTVKLQLQAL
QQNGSSVLSE DKSKRLNTIL NTMSTIYSTG KVCNPDNPQE CLLLEPGLNE
IMANSLDYNE RLWAWESWRS EVGKQLRPLY EEYVVLKNEM ARANHYEDYG
DYWRGDYEVN GVDGYDYSRG QLIEDVEHTF EEIKPLYEHL HAYVRAKLMN
AYPSYISPIG CLPAHLLGDM WGRFWTNLYS LTVPFGQKPN IDVTDAMVDQ
AWDAQRIFKE AEKFFVSVGL PNMTQGFWEN SMLTDPGNVQ KAVCHPTAWD
LGKGDFRILM CTKVTMDDFL TAHHEMGHIQ YDMAYAAQPF LLRNGANEGF
HEAVGEIMSL SAATPKHLKS IGLLSPDFQE DNETEINFLL KQALTIVGTL
PFTYMLEKWR WMVFKGEIPK DQWMKKWWEM KREIVGVVEP VPHDETYCDP
ASLFHVSNDY SFIRYYTRTL YQFQFQEALC QAAKHEGPLH KCDISNSTEA
GQKLFNMLRL GKSEPWTLAL ENVVGAKNMN VRPLLNYFEP LFTWLKDQNK
NSFVGWSTDW SPYADQSIKV RISLKSALGD KAYEWNDNEM YLFRSSVAYA
MRQYFLKVKN QMILFGEEDV RVANLKPRIS FNFFVTAPKN VSDIIPRTEV
EKAIRMSRSR INDAFRLNDN SLEFLGIQPT LGPPNQPPVS
SEQ ID NO: 30 = Wildtype SR4 nanobody amino acid seuqnence:
QVQLVESGGGLVQAGGSLRLSCAASGFPVYSWNMWWYRQAPGKEREWVAAIESHGD
STRYADSVKGRFTISRDNAKNTVYLQMNSLKPEDTAVYYCYVWVGHTYYGQGTQVT
VSAGRAGEQKLISEEDLNSAVD
SEQ ID NO: 31 Wildtype SR4 nanobody - CDR1 = GFPVYSWNMW
SEQ ID NO: 32 Wildtype SR4 nanobody - CDR2 = AIESHGDSTR
SEQ ID NO: 33 Wildtype SR4 nanobody - CDR3 = VWVGHTY
SEQ ID NO: 34 = Wildtype MR17K99Y nanobody amino acid seuqnence:
QVQLVESGGGLVQAGGSLRLSCAASGFPVEVWRMEWYRQAPGKEREGVAAIESYGHG
TRYADSVKGRFTISRDNAKNTVYLQMNSLKPEDTAVYYCNVYDDGQLAYHYDYWGQ
GTQVTVSAGRAGEQKLISEEDLNSAVD
SEQ ID NO: 35 Wildtype MR17K99Y nanobody - CDR1 = GFPVEVWRME
SEQ ID NO: 36 Wildtype MR17K99Y nanobody - CDR2 = AIESYGHGTR
SEQ ID NO: 37 Wildtype MR17K99Y nanobody - CDR3 = VYDDGQLAYHYDY
SEQ ID NO: 38 = Wildtype H11D4 nanobody amino acid seuqnence:
QVQLVESGGGLMQAGGSLRLSCAVSGRTFSTAAMGWFRQAPGKEREFVAAIRWSGGS
AYYADSVKGRFTISRDKAKNTVYLQMNSLKYEDTAVYYCARTENVRSLLSDYATWPY
DYWGQGTQVTVSSK
SEQ ID NO: 39 Wildtype H11D4 nanobody - CDR1 = GRTFSTAAMG
SEQ ID NO: 40 Wildtype H11D4 nanobody - CDR2 = AIRWSGGSAY
SEQ ID NO: 41 Wildtype H11D4 nanobody - CDR3 = RTENVRSLLSDY ATWPYDY
SEQ ID NO: 42 = Wildtype H11D4 nanobody gene seuqnence:
ATGAAATATCTGCTGCCAACCGCGGCCGCGGGTCTGCTGCTGCTGGCGGCCCAACC
AGCGATGGCGCAAGTGCAGCTGGTTGAGAGCGGCGGTGGTCTGATGCAAGCGGGTG
GTAGTCTGCGTCTGAGTTGCGCGGTTAGCGGTCGCACCTTTAGCACCGCCGCGATGG
GTTGGTTTCGCCAAGCGCCGGGCAAAGAACGCGAATTTGTTGCGGCCATCCGTTGG
AGCGGTGGTAGTGCGTACTACGCGGATAGCGTTAAAGGCCGCTTCACCATCAGCCG
CGATAAGGCGAAGAACACCGTGTATCTGCAGATGAACAGTCTGAAGTACGAGGAC
ACCGCCGTGTACTATTGCGCGCGCACCGAAAACGTTCGTAGTCTGCTGAGCGATTAC
GCCACGTGGCCGTACGATTACTGGGGTCAAGGCACCCAAGTTACCGTGAGCAGCAA
ACACCACCATCACCATCAT
SEQ ID NO: 43 = Wildtype MR17K99Y nanobody gene seuqnence:
ATGAAATATCTGCTGCCAACGGCCGCGGCGGGTCTGCTGCTGCTGGCGGCGCAACC
AGCGATGGCCCAAGTTCAGCTGGTTGAAAGCGGTGGCGGTCTGGTTCAAGCCGGTG
GTAGTCTGCGTCTGAGCTGCGCCGCCAGTGGCTTTCCGGTGGAAGTTTGGCGCATGG
AATGGTACCGCCAAGCCCCGGGCAAAGAACGCGAAGGCGTTGCCGCCATCGAAAG
CTACGGTCATGGCACCCGCTACGCCGATAGCGTTAAAGGCCGCTTCACCATCAGCC
GCGACAACGCGAAGAACACCGTGTATCTGCAGATGAACAGTCTGAAACCGGAGGA
TACGGCCGTGTACTACTGCAACGTGTACGATGATGGCCAGCTGGCGTACCATTACG
ATTACTGGGGCCAAGGCACCCAAGTTACCGTTAGTGCGGGTCGCGCGGGCGAACAG
AAGCTGATCAGCGAAGAGGATCTGAATAGCGCCGTGGATCACCATCATCATCACCA
T
SEQ ID NO: 44 = Wildtype SR4 nanobody gene seuqnence:
ATGAAATATCTGCTGCCAACCGCCGCGGCGGGTCTGCTGCTGCTGGCGGCGCAACC
AGCGATGGCCCAAGTTCAGCTGGTTGAAAGCGGTGGTGGTCTGGTTCAAGCCGGTG
GTAGTCTGCGTCTGAGCTGCGCGGCGAGTGGCTTTCCAGTGTACAGCTGGAACATGT
GGTGGTACCGCCAAGCCCCGGGTAAAGAACGCGAATGGGTTGCGGCGATCGAAAG
CCACGGCGATAGCACCCGCTACGCGGATAGCGTTAAAGGCCGCTTCACCATCAGCC
GCGACAACGCCAAGAACACCGTGTATCTGCAGATGAACAGTCTGAAACCGGAAGAT
ACCGCGGTGTACTACTGCTACGTGTGGGTTGGCCACACCTACTACGGTCAAGGCAC
CCAAGTTACCGTTAGCGCGGGTCGTGCGGGCGAACAGAAGCTGATCAGCGAGGAA
GATCTGAACAGCGCCGTGGATCACCATCACCATCATCAT
SEQ ID NO: 45
MNIPALVENQKKYFGTYSVMAMLNAQTVLDHIQKVADIEGEQNENNENLWFHPVMSH
LYNAKNGYDKQPEKTMFIIERLQSYFPFLKIMAENQREYSNGKYKQNRVEVNSNDIFEV
LKRAFGVLKMYRDLTNHYKTYEEKLNDGCEFLTSTEQPLSGMINNYYTVALRNMNER
YGYKTEDLAFIQDKRFKFVKDAYGKKKSQVNTGFFLSLQDYNGDTQKKLHLSGVGIAL
LICLFLDKQYINIFLSRLPIFSSYNAQSEERRIIIRSFGINSIKLPKDRIHSEKSNKSVAMDML
NEVKRCPDELFTTLSAEKQSRFRIISDDHNEVLMKRSSDRFVPLLLQYIDYGKLFDHIRF
HVNMGKLRYLLKADKTCIDGQTRVRVIEQPLNGFGRLEEAETMRKQENGTFGNSGIRIR
DFENMKRDDANPANYPYIVDTYTHYILENNKVEMFINDKEDSAPLLPVIEDDRYVVKTI
PSCRMSTLEIPAMAFHMFLFGSKKTEKLIVDVHNRYKRLFQAMQKEEVTAENIASFGIA
ESDLPQKILDLISGNAHGKDVDAFIRLTVDDMLTDTERRIKRFKDDRKSIRSADNKMGK
RGFKQISTGKLADFLAKDIVLFQPSVNDGENKITGLNYRIMQSAIAVYDSGDDYEAKQQ
FKLMFEKARLIGKGTTEPHPFLYKVFARSIPANAVEFYERYLIERKFYLTGLSNEIKKGN
RVDVPFIRRDQNKWKTPAMKTLGRIYSEDLPVELPRQMFDNEIKSHLKSLPQMEGIDFN
NANVTYLIAEYMKRVLDDDFQTFYQWNRNYRYMDMLKGEYDRKGSLQHCFTSVEER
EGLWKERASRTERYRKQASNKIRSNRQMRNASSEEIETILDKRLSNSRNEYQKSEKVIRR
YRVQDALLFLLAKKTLTELADFDGERFKLKEIMPDAEKGILSEIMPMSFTFEKGGKKYTI
TSEGMKLKNYGDFFVLASDKRIGNLLELVGSDIVSKEDIMEEFNKYDQCRPEISSIVFNL
EKWAFDTYPELSARVDREEKVDFKSILKILLNNKNINKEQSDILRKIRNAFDHNNYPDK
GVVEIKALPEIAMSIKKAFGEYAIMKGSLQLPPLERLTLGSSYPYDVPDYAYPYDVPDY
AYPYDVPDYA
SEQ ID NO: 46
MNIPALVENQKKYFGTYSVMAMLNAQTVLDHIQKVADIEGEQNENNENLWFHPVMSH
LYNAKNGYDKQPEKTMFIIERLQSYFPFLKIMAENQREYSNGKYKQNRVEVNSNDIFEV
LKRAFGVLKMYRDLTNAYKTYEEKLNDGCEFLTSTEQPLSGMINNYYTVALRNMNER
YGYKTEDLAFIQDKRFKFVKDAYGKKKSQVNTGFFLSLQDYNGDTQKKLHLSGVGIAL
LICLFLDKQYINIFLSRLPIFSSYNAQSEERRIIIRSFGINSIKLPKDRIHSEKSNKSVAMDML
NEVKRCPDELFTTLSAEKQSRFRIISDDHNEVLMKRSSDRFVPLLLQYIDYGKLFDHIRF
HVNMGKLRYLLKADKTCIDGQTRVRVIEQPLNGFGRLEEAETMRKQENGTFGNSGIRIR
DFENMKRDDANPANYPYIVDTYTHYILENNKVEMFINDKEDSAPLLPVIEDDRYVVKTI
PSCRMSTLEIPAMAFHMFLFGSKKTEKLIVDVHNRYKRLFQAMQKEEVTAENIASFGIA
ESDLPQKILDLISGNAHGKDVDAFIRLTVDDMLTDTERRIKRFKDDRKSIRSADNKMGK
RGFKQISTGKLADFLAKDIVLFQPSVNDGENKITGLNYRIMQSAIAVYDSGDDYEAKQQ
FKLMFEKARLIGKGTTEPHPFLYKVFARSIPANAVEFYERYLIERKFYLTGLSNEIKKGN
RVDVPFIRRDQNKWKTPAMKTLGRIYSEDLPVELPRQMFDNEIKSHLKSLPQMEGIDEN
NANVTYLIAEYMKRVLDDDFQTFYQWNRNYRYMDMLKGEYDRKGSLQHCFTSVEER
EGLWKERASRTERYRKQASNKIRSNRQMRNASSEEIETILDKRLSNSRNEYQKSEKVIRR
YRVQDALLFLLAKKTLTELADFDGERFKLKEIMPDAEKGILSEIMPMSFTFEKGGKKYTI
TSEGMKLKNYGDFFVLASDKRIGNLLELVGSDIVSKEDIMEEFNKYDQCRPEISSIVFNL
EKWAFDTYPELSARVDREEKVDFKSILKILLNNKNINKEQSDILRKIRNAFDHNNYPDK
GVVEIKALPEIAMSIKKAFGEYAIMKGSLQLPPLERLTLGSSYPYDVPDYAYPYDVPDY
AYPYDVPDYA
SEQ ID NO: 47
MNIPALVENQKKYFGTYSVMAMLNAQTVLDHIQKVADIEGEQNENNENLWFHPVMSH
LYNAKNGYDKQPEKTMFIIERLQSYFPFLKIMAENQREYSNGKYKQNRVEVNSNDIFEV
LKRAFGVLKMYRDLTNHYKTYEEKLNDGCEFLTSTEQPLSGMINNYYTVALRNMNER
YGYKTEDLAFIQDKRFKFVKDAYGKKKSQVNTGFFLSLQDYNGDTQKKLHLSGVGIAL
LICLFLDKQYINIFLSRLPIFSSYNAQSEERRIIIRSFGINSIKLPKDRIHSEKSNKSVAMDML
NEVKRCPDELFTTLSAEKQSRFRIISDDHNEVLMKRSSDRFVPLLLQYIDYGKLFDHIRF
HVNMGKLRYLLKADKTCIDGQTRVRVIEQPLNGFGRLEEAETMRKQENGTFGNSGIRIR
DFENMKRDDANPANYPYIVDTYTHYILENNKVEMFINDKEDSAPLLPVIEDDRYVVKTI
PSCRMSTLEIPAMAFHMFLFGSKKTEKLIVDVHNRYKRLFQAMQKEEVTAENIASFGIA
ESDLPQKILDLISGNAHGKDVDAFIRLTVDDMLTDTERRIKRFKDDRKSIRSADNKMGK
RGFKQISTGKLADFLAKDIVLFQPSVNDGENKITGLNYRIMQSAIAVYDSGDDYEAKQQ
FKLMFEKARLIGKGTTEPHPFLYKVFARSIPANAVEFYERYLIERKFYLTGLSNEIKKGN
RVDVPFIRRDQNKWKTPAMKTLGRIYSEDLPVELPRQMFDNEIKSHLKSLPQMEGIDFN
NANVTYLIAEYMKRVLDDDFQTFYQWNRNYRYMDMLKGEYDRKGSLQHCFTSVEER
EGLWKERASRTERYRKQASNKIRSNRQMRNASSEEIETILDKRLSNSRNEYQKSEKVIRR
YRVQDALLFLLAKKTLTELADFDGERFKLKEIMPDAEKGILSEIMPMSFTFEKGGKKYTI
TSEGMKLKNYGDFFVLASDKRIGNLLELVGSDIVSKEDIMEEFNKYDQCRPEISSIVFNL
EKWAFDTYPELSARVDREEKVDFKSILKILLNNKNINKEQSDILRKIRNAFDANNYPDK
GVVEIKALPEIAMSIKKAFGEYAIMKGSLQLPPLERLTLGSSYPYDVPDYAYPYDVPDY
AYPYDVPDYA
SEQ ID NO: 48
MNIPALVENQKKYFGTYSVMAMLNAQTVLDHIQKVADIEGEQNENNENLWFHPVMSH
LYNAKNGYDKQPEKTMFIIERLQSYFPFLKIMAENQREYSNGKYKQNRVEVNSNDIFEV
LKRAFGVLKMYRDLTNAYKTYEEKLNDGCEFLTSTEQPLSGMINNYYTVALRNMNER
YGYKTEDLAFIQDKRFKFVKDAYGKKKSQVNTGFFLSLQDYNGDTQKKLHLSGVGIAL
LICLFLDKQYINIFLSRLPIFSSYNAQSEERRIIIRSFGINSIKLPKDRIHSEKSNKSVAMDML
NEVKRCPDELFTTLSAEKQSRFRIISDDHNEVLMKRSSDRFVPLLLQYIDYGKLFDHIRF
HVNMGKLRYLLKADKTCIDGQTRVRVIEQPLNGFGRLEEAETMRKQENGTFGNSGIRIR
DFENMKRDDANPANYPYIVDTYTHYILENNKVEMFINDKEDSAPLLPVIEDDRYVVKTI
PSCRMSTLEIPAMAFHMFLFGSKKTEKLIVDVHNRYKRLFQAMQKEEVTAENIASFGIA
ESDLPQKILDLISGNAHGKDVDAFIRLTVDDMLTDTERRIKRFKDDRKSIRSADNKMGK
RGFKQISTGKLADFLAKDIVLFQPSVNDGENKITGLNYRIMQSAIAVYDSGDDYEAKQQ
FKLMFEKARLIGKGTTEPHPFLYKVFARSIPANAVEFYERYLIERKFYLTGLSNEIKKGN
RVDVPFIRRDQNKWKTPAMKTLGRIYSEDLPVELPRQMFDNEIKSHLKSLPQMEGIDFN
NANVTYLIAEYMKRVLDDDFQTFYQWNRNYRYMDMLKGEYDRKGSLQHCFTSVEER
EGLWKERASRTERYRKQASNKIRSNRQMRNASSEEIETILDKRLSNSRNEYQKSEKVIRR
YRVQDALLFLLAKKTLTELADFDGERFKLKEIMPDAEKGILSEIMPMSFTFEKGGKKYTI
TSEGMKLKNYGDFFVLASDKRIGNLLELVGSDIVSKEDIMEEFNKYDQCRPEISSIVFNL
EKWAFDTYPELSARVDREEKVDFKSILKILLNNKNINKEQSDILRKIRNAFDANNYPDK
GVVEIKALPEIAMSIKKAFGEYAIMKGSLQLPPLERLTLGSSYPYDVPDYAYPYDVPDY
AYPYDVPDYA
SEQ ID NO: 49 = mFSYRS amino acid sequence, where bold/underlined refers to
mutated residues
DKKPLNTLISATGLWMSRTGTIHKIKHHEVSRSKIYIEMACGDHLVVNNSRSSRTARALR
HHKYRKTCKRCRVSDEDLNKFLTKANEDQTSVKVKVVSAPTRTKKAMPKSVARAPKPL
ENTEAAQAQPSGSKFSPAIPVSTQESVSVPASVSTSISSISTGATASALVKGNTNPITSMSA
PVQASAPALTKQTDRLEVLLNPKDEISLNSGKPFRELESELLSRRKKDLQQIYAEERENY
LGKLEREITRFFVDRGFLEIKSPILIPLEYIERMGIDNDTELSKQIFRVDKNFCLRPMLAPN
MYNYLRKLDRALPDPIKTFEIGPCYRKESDGKEHLEEFTMLGFCQMGSGCTRENLESIIT
DFLNHLGIDFKIVGDSCMVYGDTLDVMHGDLELSSAVVGPIPLDREWGIDKPWIGAGFG
LERLLKVKHDFKNIKRAARSESYYNGISTNL
SEQ ID NO: 50 = pBAD-dZHER2-D36/D37TAG, wherein bold/underlined refers to
amber codon TAG at 36^{th}/37^{th} position
MAVDNKFNKEMRNAYWEIALLPNLNNQQKRAFIRSLYDDPSQSANLLAEAKKLNDAQ
APKVEVDNKFNKEMRNAYWEIALLPNLNNQQKRAFIRSLYDDPSQSANLLAEAKKLND
AQAPKHHHHHH
SEQ ID NO: 51 = pBAD-NbHER2-Y37TAG, where bold/underlined refers to amber
codon TAG at 37^{th} position.
MKYLLPTAAAGLLLLAAQPAMAMGQVQLQESGGGSVQAGGSLKLTCAASGYIFNSCG
MGWYRQSPGRERELVSRISGDGDTWHKESVKGRFTISQDNVKKTLYLQMNSLKPEDTA
VYFCAVCYN LETYWGQGTQVTVSSHHHHHH
SEQ ID NO: 52 = pBR322-TrasFab-S50/Y92TAG = Light Chain, where
bold/underlined refers to amber codon TAG at 50^{th}/92^{nd} position of the Light Chain.
MKSLLPTAAAGLLLLAAQPAMASDIQMTQSPSSLSASVGDRVTITCRASQDVNTAVAW
YQQKPGKAPKLLIYSASFLYSGVPSRFSGSRSGTDFTLTISSLQPEDFATYYCQQHYTTPP
TFGQGTKVEIKRTVAAPSVFIFPPSDEQLKSGTASVVCLLNNFYPREAKVQWKVDNALQ
SGNSQESVTEQD SKDSTYSLSSTLTLSKADYEKHKVYACEVTHQGLSSPVTKSFNRGEC
SEQ ID NO: 53 = pBR322-TrasFab-S50/Y92TAG = Heavy Chain
MKKNIAFLLASMFVFSIATNAYAEISEVQLVESGGGLVQPGGSLRLSCAASGFNIKDTYI
HWVRQAPGKGLEWVARIYPTNGYTRYADSVKGRFTISADTSKNTAYLQMNSLRAEDTA
VYYCSRWGGDGFYALDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPS
NTKVDKKVEPKSCDKTHTGGSGSAGGLNDIFEAQKIEWHE
SEQ ID NO: 54 = pBAD-NbEGFR-Q116TAG, where bold/underlined refers to amber
codon TAG at 116^{th} position
MKYLLPTAAAGLLLLAAQPAMAMGQVKLEESGGGSVQTGGSLRLTCAASGRTSRSYG
MGWFRQAPGKEREFVSGISWRGDSTGYADSVKGRFTISRDNAKNTVDLQMNSLKPEDT
AIYYCAAAAGSAWYGTLYEYDYWGQGTQVTVSSHHHHHH
SEQ ID NO: 55 = pET-32a-Tx-NRG1b-A53TAG, where bold/underlined refers to
amber codon TAG at 53^{rd} position.
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSGSGLEVL
FQGPSHLVKCAEKEKTFCVNGGECFMVKDLSNPSRYLCKCPNEFTGDRCQNYVMASFY
KHLGIEGSGSGSDYK DDDDKAAALEHHHHHH
SEQ ID NO: 56
MDKKPLNTLISATGLWMSRTGTIHKIKHHEVSRSKIYIEMACGDHLVVNNSRSSRTARA
LRHHKYRKTCKRCRVSDEDLNKFLTKANEDQTSVKVKVVSAPTRTKKAMPKSVARAP
KPLENTEAAQAQPSGSKFSPAIPVSTQESVSVPASVSTSISSISTGATASALVKGNTNPITS
MSAPVQASAPALTKSQTDRLEVLLNPKDEISLNSGKPFRELESELLSRRKKDLQQIYAEE
RENYLGKLEREITRFFVDRGFLEIKSPILIPLEYIERMGIDNDTELSKQIFRVDKNFCLRPM
LAPNLYNYLRKLDRALPDPIKIFEIGPCYRKESDGKEHLEEFTMLNFCQMGSGCTRENLE
SIITDFLNHLGIDFKIVGDSCMVYGDTLDVMHGDLELSSAVVGPIPLDREWGIDKPWIGA
GFGLERLLKVKHDFKNIKRAARSESYYNGISTNL*
SEQ ID NO: 57
ATGGATAAAAAGCCTTTGAACACTCTGATTTCTGCGACCGGTCTGTGGATGTCCCGC
ACCGGCACCATCCACAAAATCAAACACCATGAAGTTAGCCGTTCCAAAATCTACAT
TGAAATGGCTTGCGGCGATCACCTGGTTGTCAACAACTCCCGTTCTTCTCGTACCGC
TCGCGCACTGCGCCACCACAAATATCGCAAAACCTGCAAACGTTGCCGTGTTAGCG
ATGAGGACCTGAACAAATTCCTGACCAAAGCTAACGAGGATCAGACCTCCGTAAAA
GTGAAGGTAGTAAGCGCTCCGACCCGTACTAAAAAGGCTATGCCAAAAAGCGTGGC
CCGTGCCCCGAAACCTCTGGAAAACACCGAGGCGGCTCAGGCTCAACCATCCGGTT
CTAAATTTTCTCCGGCGATCCCAGTGTCCACCCAAGAATCTGTTTCCGTACCAGCAA
GCGTGTCTACCAGCATTAGCAGCATTTCTACCGGTGCTACCGCTTCTGCGCTGGTAA
AAGGTAACACTAACCCGATTACTAGCATGTCTGCACCGGTACAGGCAAGCGCCCCA
GCTCTGACTAAATCCCAGACGGACCGTCTGGAGGTGCTGCTGAACCCAAAGGATGA
AATCTCTCTGAACAGCGGCAAGCCTTTCCGTGAGCTGGAAAGCGAGCTGCTGTCTC
GTCGTAAAAAGGATCTGCAACAGATCTACGCTGAGGAACGCGAGAACTATCTGGGT
AAGCTGGAGCGCGAAATTACTCGCTTCTTCGTGGATCGCGGTTTCCTGGAGATCAAA
TCTCCGATTCTGATTCCGCTGGAATACATTGAACGTATGGGCATCGATAATGATACC
GAACTGTCTAAACAGATCTTCCGTGTGGATAAAAACTTCTGTCTGCGTCCGATGCTG
ATTCCGAACTTGTACAACTATTTACGTAAACTGGACCGTGCCCTGCCGGACCCGATC
AAAATATTCGAGATCGGTCCTTGCTACCGTAAAGAGTCCGACGGTAAAGAGCACCT
GGAAGAATTCACCATGCTGACATTCATTCAGATGGGTAGCGGTTGCACGCGTGAAA
ACCTGGAATCCATTATCACCGACTTCCTGAATCACCTGGGTATCGATTTCAAAATTG
TTGGTGACAGCTGTATGGTGTTAGGCGATACGCTGGATGTTATGCACGGCGATCTGG
AGCTGTCTTCCGCAGTTGTGGGCCCAATCCCGCTGGATCGTGAGTGGGGTATCGACA
AACCTAAAATCGGTGCGGGTTTTGGTCTGGAGCGTCTGCTGAAAGTAAAACACGAC
TTCAAGAACATCAAACGTGCTGCACGTTCCGAGTCCTATTACAATGGTATTTCTACT
AACCTGTAA
SEQ ID NO: 58
MDKKPLNTLISATGLWMSRTGTIHKIKHHEVSRSKIYIEMACGDHLVVNNSRSSRTARA
LRHHKYRKTCKRCRVSDEDLNKFLTKANEDQTSVKVKVVSAPTRTKKAMPKSVARAP
KPLENTEAAQAQPSGSKFSPAIPVSTQESVSVPASVSTSISSISTGATASALVKGNTNPITS
MSAPVQASAPALTKSQTDRLEVLLNPKDEISLNSGKPFRELESELLSRRKKDLQQIYAEE
RENYLGKLEREITRFFVDRGFLEIKSPILIPLEYIERMGIDNDTELSKQIFRVDKNFCLRPM
LIPNLYNYLRKLDRALPDPIKIFEIGPCYRKESDGKEHLEEFTMLTFIQMGSGCTRENLESI
ITDFLNHLGIDFKIVGDSCMVLGDTLDVMHGDLELSSAVVGPIPLDREWGIDKPKIGA
SEQ ID NO: 59 =
ggaaacctgatcatgtagatcgaatggactctaaatccgttcagccgggttagattcccggggtttccg
SEQ ID NO: 60 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYGDYWG QGTQVTVSS
SEQ ID NO: 61 = GYIFGRNAMG = CDR1, mNb6 nanobody
SEQ ID NO: 62 = GITRRGSITY = CDR2, mNb6
SEQ ID NO: 63 = DPASPAYGDY = CDR3, mNb6
SEQ ID NO: 64 = DPASPAYGDX^{FFY} = CDR3, mNb6
SEQ ID NO: 65 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYGDX^{FFY}WG QGTQVTVSS
SEQ ID NO: 66 - Nanobody 2rs15d (Nb_{Her2})
QVQLQESGGG SVQAGGSLKL TCAASGYIFN SCGMGWYRQS PGRERELVSR
ISGDGDTWHK ESVKGRFTIS QDNVKKTLYL QMNSLKPEDT AVYFCAVCYN
LETYWGQGTQ VTVSS
SEQ ID NO: 67 - Nanobody 2rs15d CDR1 = GYIFNSCG
SEQ ID NO: 68 - Nanobody 2rs15d CDR2 = RISGDGD
SEQ ID NO: 69 - Nanobody 2rs15d CDR3 = AVCYNLETY
SEQ ID NO: 70 - Nanobody 2rs15d CDR2 = RISGX^{FSY}GD
SEQ ID NO: 71 - Nanobody 2rs15d CDR3 = AVCYNLX^{FSY}TY
SEQ ID NO: 72 - Nanobody 2rs15d (Nb_{Her2}) - 2rs15D(D54FSY)
QVQLQESGGG SVQAGGSLKL TCAASGYIFN SCGMGWYRQS PGRERELVSR
ISGX^{FSY}GDTWHK ESVKGRFTIS QDNVKKTLYL QMNSLKPEDT AVYFCAVCYN
LETYWGQGTQ VTVSS
SEQ ID NO: 73 - Nanobody 2rs15d (Nb_{Her2}) - 2rs15D(E102FSY)
QVQLQESGGG SVQAGGSLKL TCAASGYIFN SCGMGWYRQS PGRERELVSR
ISGDGDTWHK ESVKGRFTIS QDNVKKTLYL QMNSLKPEDT AVYFCAVCYN
LX^{FSY}TYWGQGTQ VTVSS
SEQ ID NO: 74 - Nanobody C21
EVQLVESGGE LVQAGGSLRL SCAASGLTFS SYNMGWFRRA PGKEREFVAS
ITWSGRDTFY ADSVKGRFTI SRDNAKNTVY LQMSSLKPED TAVYYCAANP
WPVAAPRSGT YWGQGTQVTV SS
SEQ ID NO: 75 - Nanobody C21 CDR1 = GLTFSSYN
SEQ ID NO: 76 - Nanobody C21 CDR2 = ITWSGRDT
SEQ ID NO: 77 - Nanobody C21 CDR3 = AANPWPVAAPRSGTY
SEQ ID NO: 78 - Nanobody C21 CDR1 = GLTFSX^{FSY}YN
SEQ ID NO: 79 - Nanobody C21
EVQLVESGGE LVQAGGSLRL SCAASGLTFS X^{FSY}YNMGWFRRA PGKEREFVAS
ITWSGRDTFY ADSVKGRFTI SRDNAKNTVY LQMSSLKPED TAVYYCAANP
WPVAAPRSGT YWGQGTQVTV SS
SEQ ID NO: 80 = Nanobody NB13
QVQLQESGGG SVQTGGSLRL SCAASGYTAS FSWIGYFRQA PGKEREGVAV
INVGVGSTYY ADSVKGRFTI SRDNTENTIS LEMNSLKPED TGLYYCAGSL
RWSRPPNPIS EDAYNYWGQG TQVTVSS
SEQ ID NO: 81 = Nanobody NB13 CDR1 = GYTASFSWI
SEQ ID NO: 82 = Nanobody NB13 CDR2 = VINVGVGST
SEQ ID NO: 83 = Nanobody NB13 CDR3 = AGSLRWSRPPNPISEDAYNY
SEQ ID NO: 84 = Nanobody NB13 CDR2 = VINVX^{FSY}VGST
SEQ ID NO: 85 = Nanobody NB13 CDR2 = VINVGVGX^{FSY}T
SEQ ID NO: 86 = Nanobody NB13 CDR1 = GYTASFX^{mFSY}WI
SEQ ID NO: 87 = Nanobody NB13 CDR2 = VINVX^{mFSY}VGST
SEQ ID NO: 88 = Nanobody NB13
QVQLQESGGG SVQTGGSLRL SCAASGYTAS FSWIGYFRQA PGKEREGVAV
INVX^{FSY}VGSTYY ADSVKGRFTI SRDNTENTIS LEMNSLKPED TGLYYCAGSL
RWSRPPNPIS EDAYNYWGQG TQVTVSS
SEQ ID NO: 89 = Nanobody NB13
QVQLQESGGG SVQTGGSLRL SCAASGYTAS FSWIGYFRQA PGKEREGVAV
INVGVGX^{FSY}TYY ADSVKGRFTI SRDNTENTIS LEMNSLKPED TGLYYCAGSL
RWSRPPNPIS EDAYNYWGQG TQVTVSS
SEQ ID NO: 90 = Nanobody NB13
QVQLQESGGG SVQTGGSLRL SCAASGYTAS FX^{mFSY}WIGYFRQA PGKEREGVAV
INVGVGSTYY ADSVKGRFTI SRDNTENTIS LEMNSLKPED TGLYYCAGSL
RWSRPPNPIS EDAYNYWGQG TQVTVSS
SEQ ID NO: 91 = Nanobody NB13
QVQLQESGGG SVQTGGSLRL SCAASGYTAS FSWIGYFRQA PGKEREGVAV
INVX^{mFSY}VGSTYY ADSVKGRFTI SRDNTENTIS LEMNSLKPED TGLYYCAGSL
RWSRPPNPIS EDAYNYWGQG TQVTVSS
SEQ ID NO: 92 - Nanobody NB17B05 (alternatively “MS83”)
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TRGSSDYWGQ GTLVTVSSAA AEQKLISEED LNGAA
SEQ ID NO: 93 = LNAMA
SEQ ID NO: 94 = GIFGVGSTRYADSVKG
SEQ ID NO: 95 = SSVTRGSSDY
SEQ ID NO: 96 = GIFGVGSTRX^{FSY}ADSVKG
SEQ ID NO: 97 = GIFGVGSTRYX^{FSY}DSVKG
SEQ ID NO: 98 = GIFGVGSTRYAX^{FSY}SVKG
SEQ ID NO: 99 = GIFGVGSTRYADX^{FSY}VKG
SEQ ID NO: 100 = GIFGVGSTRYADSX^{FSY}KG
SEQ ID NO: 101 = GIFGVGSTRYADSVX^{FSY}G
SEQ ID NO: 102 = GIFGVGSTRYADSVKX^{FSY}
SEQ ID NO: 103 = SSVTX^{FSY}GSSDY
SEQ ID NO: 104 = SSVTRX^{FSY}SSDY
SEQ ID NO: 105 = GIFGVGSX^{mFSY}RYADSVKG
SEQ ID NO: 106 = GIFGVGSTX^{mFSY}YADSVKG
SEQ ID NO: 107 = GIFGVGSTRX^{mFSY}ADSVKG
SEQ ID NO: 108 = GIFGVGSTRYX^{mFSY}DSVKG
SEQ ID NO: 109 = GIFGVGSTRYAX^{mFSY}SVKG
SEQ ID NO: 110 = GIFGVGSTRYADX^{mFSY}VKG
SEQ ID NO: 111 = GIFGVGSTRYADSX^{mFSY}KG
SEQ ID NO: 112 = GIFGVGSTRYADSVX^{mFSY}G
SEQ ID NO: 113 = GIFGVGSTRYADSVKX^{mFSY}
SEQ ID NO: 114 = SSVTX^{mFSY}GSSDY
SEQ ID NO: 115 = SSVTRX^{mFSY}SSDY
SEQ ID NO: 116 - Nanobody NB17B05
EVQLVESGGG LVQPGGSLRL SCAASGSIGG LNAMAWYRQA PGKERELVAG
IFGVGSTRX^{FSY}A DSVKGRFTIS RDNSKNTVYL QMNSLRSEDT AVYYCAMSSV
TRGSSDYWGQ GTLVTVSSAA AEQKLISEED LNGAA
SEQ ID NO: 117- Nanobody NB17B05
EVQLVESGGG LVQPGGSLRL SCAASGSIGG LNAMAWYRQA PGKERELVAG
IFGVGSTRYX^{FSY} DSVKGRFTIS RDNSKNTVYL QMNSLRSEDT AVYYCAMSSV
TRGSSDYWGQ GTLVTVSSAA AEQKLISEED LNGAA
SEQ ID NO: 118 - Nanobody NB17B05
EVQLVESGGG LVQPGGSLRL SCAASGSIGG LNAMAWYRQA PGKERELVAG
IFGVGSTRYA X^{FSY}SVKGRFTIS RDNSKNTVYL QMNSLRSEDT AVYYCAMSSV
TRGSSDYWGQ GTLVTVSSAA AEQKLISEED LNGAA
SEQ ID NO: 119 - Nanobody NB17B05
EVQLVESGGG LVQPGGSLRL SCAASGSIGG LNAMAWYRQA PGKERELVAG
IFGVGSTRYA DX^{FSY}VKGRFTIS RDNSKNTVYL QMNSLRSEDT AVYYCAMSSV
TRGSSDYWGQ GTLVTVSSAA AEQKLISEED LNGAA
SEQ ID NO: 120 - Nanobody NB17B05
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TRX^{FSY}SSDYWGQ GTLVTVSSAA AEQKLISEED LNGAA
SEQ ID NO: 121 - Nanobody NB17B05
EVQLVESGGG LVQPGGSLRL SCAASGSIGG LNAMAWYRQA PGKERELVAG
IFGVGSX^{mFSY}RYA DSVKGRFTIS RDNSKNTVYL QMNSLRSEDT AVYYCAMSSV
TRGSSDYWGQ GTLVTVSSAA AEQKLISEED LNGAA
SEQ ID NO: 122 - Nanobody NB17B05
EVQLVESGGG LVQPGGSLRL SCAASGSIGG LNAMAWYRQA PGKERELVAG
IFGVGSTX^{mFSY}YA DSVKGRFTIS RDNSKNTVYL QMNSLRSEDT AVYYCAMSSV
TRGSSDYWGQ GTLVTVSSAA AEQKLISEED LNGAA
SEQ ID NO: 123 - Nanobody NB17B05
EVQLVESGGG LVQPGGSLRL SCAASGSIGG LNAMAWYRQA PGKERELVAG
IFGVGSTRX^{mFSY}A DSVKGRFTIS RDNSKNTVYL QMNSLRSEDT AVYYCAMSSV
TRGSSDYWGQ GTLVTVSSAA AEQKLISEED LNGAA
SEQ ID NO: 124 - Nanobody NB17B05
EVQLVESGGG LVQPGGSLRL SCAASGSIGG LNAMAWYRQA PGKERELVAG
IFGVGSTRYA DSVX^{mFSY}GRFTIS RDNSKNTVYL QMNSLRSEDT AVYYCAMSSV
TRGSSDYWGQ GTLVTVSSAA AEQKLISEED LNGAA
SEQ ID NO: 125 - Nanobody NB17B05
EVQLVESGGG LVQPGGSLRL SCAASGSIGG LNAMAWYRQA PGKERELVAG
IFGVGSTRYA DSVKX^{mFSY}RFTIS RDNSKNTVYL QMNSLRSEDT AVYYCAMSSV
TRGSSDYWGQ GTLVTVSSAA AEQKLISEED LNGAA
SEQ ID NO: 126 - Nanobody NB17B05
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TX^{mFSY}GSSDYWGQ GTLVTVSSAA AEQKLISEED LNGAA
SEQ ID NO: 127 - Nanobody NB17B05
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TRX^{mFSY}SSDYWGQ GTLVTVSSAA AEQKLISEED LNGAA
SEQ ID NO: 128 - MKYLLPTAAA GLLLLAAQPA MAMA = optionally present at
the N-terminus of nanobody NB17B05
SEQ ID NO: 129 - MS211
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SS

In the sequences below, the glycine-serine peptide linker is underlined.

SEQ ID NO: 130 - Dimer 10-60FSY (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGGSGGGGSEVQLVESGGGLVQPGGSLRLSCAASGSIGGLNAMAWYRQAPGKEREL
VAGIFGVGSTRYX^{FSY}DSVKGRFTISRDNSKNTVYLQMNSLRSEDTAVYYCRMSSVTRGSS
DYWGQGTLVTVSSAAAEQKLISEEDLNGAA In embodiments, the dimer further comprises
SEQ ID NO: 128 at the N-terminus
SEQ ID NO: 131 - Dimer12-60FSY - (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGSGGGSGGGSEVQLVESGGGLVQPGGSLRLSCAASGSIGGLNAMAWYRQAPGKER
ELVAGIFGVGSTRYX^{FSY}DSVKGRFTISRDNSKNTVYLQMNSLRSEDTAVYYCRMSSVTRGS
SDYWGQGTLVTVSSAAAEQKLISEEDLNGAA In embodiments, the dimer further comprises
SEQ ID NO: 128 at the N-terminus
SEQ ID NO: 132 - Dimer15-60FSY - (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGGSGGGGSGGGGSEVQLVESGGGLVQPGGSLRLSCAASGSIGGLNAMAWYRQAPG
KERELVAGIFGVGSTRYX^{FSY}DSVKGRFTISRDNSKNTVYLQMNSLRSEDTAVYYCRMSSV
TRGSSDYWGQGTLVTVSSAAAEQKLIS EEDLNGAA In embodiments, the dimer further
comprises SEQ ID NO: 128 at the N-terminus
SEQ ID NO: 133 - Dimer20-60FSY - (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGGSGGGGSGGGGSGGGGSEVQLVESGGGLVQPGGSLRLSCAASGSIGGLNAMAWY
RQAPGKERELVAGIFGVGSTRYX^{FSY}DSVKGRFTISRDNSKNTVYLQMNSLRSEDTAVYYC
RMSSVTRGSSDYWGQGTLVTVSSAAA EQKLISEEDLNGAA In embodiments, the dimer
further comprises SEQ ID NO: 128 at the N-terminus
SEQ ID NO: 134 - 7D12 wt-2rs15D wt
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYW
GQGTQVTVSSGGSGGGGGGSQVQLQESGGGSVQAGGSLKLTCAASGYIFNSCGMGWY
RQSPGRERELVSRISGDGDTWHKESVKGRFTISQDNVKKTLYLQM NSLKPEDTAVYFC
AVCYNLETYWGQGTQVTVSS
SEQ ID NO: 135 - 7D12 wt-2rs15D D54(FSY)
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYW
GQGTQVTVSSGGSGGGGGGSQVQLQESGGGSVQAGGSLKLTCAASGYIFNSCGMGWY
RQSPGRERELVSRISGX^{FSY}GDTWHKESVKGRFTISQDNVKKTLYLQMNSLKPEDTAVYF
CAVCYNLETYWGQGTQVTVSS
SEQ ID NO: 136 - 7D12 Y109(FSY)-2rs15D D54(FSY)
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLX^{FS}YEYDY
WGQGTQVTVSSGGSGGGGGGSQVQLQESGGGSVQAGGSLKLTCAASGYIFNSCGMG
WYRQSPGRERELVSRISGX^{FSY}GDTWHKESVKGRFTISQDNVKKTLYLQM
NSLKPEDTAVYFC AVCYNLETYWGQGTQVTVSS
SEQ ID NO: 137 - ZHER2: 2891
AEAKYAKEMR NAYWEIALLP NLTNQQKRAF IRKLYDDPSQ SSELLSEAKKLNDSQAPK
SEQ ID NO: 138 - ZHER2: 342
VDNKFNKEMR NAYWEIALLP NLNNQQKRAF IRSLYDDPSQ SANLLAEAKK
LNDAQAPK
SEQ ID NO: 139 - F57
EVQLVESGGGLVQAGGSLRLSCAASGITFSINTMGWYRQAPGKQRELVALISSIGDTYY
ADSVKGRFTISRDNAKNTVYLQMNSLKPEDTAVYYCKRFRTAAQGTDYWGQGTQVTV
SS
SEQ ID NO: 140 - 7D12 wt-ZHER2: 2891 wt
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLYEYDYWGQG
TQVTVSSGGSGGGGGGSAEAKYAKEMRNAYWEIALLPNLTNQQKRAFIRKLYDDPSQSS
ELLSEAKKLNDSQAPK
SEQ ID NO: 141 - 7D12 Y109(FSY)-ZHER2: 2891
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLX^{FSY}EYDYW
GQGTQVTVSSGGSGGGGGGSAEAKYAKEMRNAYWEIALLPNLTNQQKRAFIRKLYDDP
SQSSELLSEAKKLNDSQAPK
SEQ ID NO: 142 - 7D12 wt-ZHER2: 342 wt
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLYEYDYWGQ
GTQVTVSSGGSGGGGGGSVDNKFNKEMRNAYWEIALLPNLNNQQKRAFIRSLYDDPSQS
ANLLAEAKKLNDAQAPK
SEQ ID NO: 143 - 7D12 Y109(FSY)-ZHER2: 342 wt
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLX^{FSY}EYDYW
GQGTQVTVSSGGSGGGGGGSVDNKFNKEMRNAYWEIALLPNLNNQQKRAFIRSLYDDP
SQSANLLAEAKKLNDAQAPK
SEQ ID NO: 144 - 7D12 Y109(FSY)-ZHER2: 342 D37
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLX^{FSY}EYDYW
GQGTQVTVSSGGSGGGGGGSVDNKFNKEMRNAYWEIALLPNLNNQQKRAFIRSLYDX^{FSY}
PSQSANLLAEAKKLNDAQAPK
SEQ ID NO: 145 - 7D12 wt- 5F7
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYW
GQGTQVTVSSGGSGGGGGGSEVQLVESGGGLVQAGGSLRLSCAASGITFSINTMGWYR
QAPGKQRELVALISSIGDTYYADSVKGRFTISRDNAKNTVYLQMNSLKPEDTAVYYCK
RFRTAAQGTDYWGQGTQVTVSS
SEQ ID NO: 146 - 7D12 Y109(FSY)-5F7
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLX^{FSY}EYDY
WGQGTQVTVSSGGSGGGGGGSEVQLVESGGGLVQAGGSLRLSCAASGITFSINTMGW
YRQAPGKQRELVALISSIGDTYYADSVKGRFTISRDNAKNTVYLQMNSLKPEDTAVYY
CKRFRTAAQGTDYWGQGTQVTVSS
SEQ ID NO: 147 - ZHER2: 342 wt-ZHER2: 342 wt-2rs15d wt
VDNKFNKEMRNAYWEIALLPNLNNQQKRAFIRSLYDDPSQSANLLAEAKKLNDAQAPKV
DNKFNKEMRNAYWEIALLPNLNNQQKRAFIRSLYDDPSQSANLLAEAKKLNDAQAPKGG
SGGGGGGSQVQLQESGGGSVQAGGSLKLTCAASGYIFNSCGMGWYRQSPGRERELVSRIS
GDGDTWHKESVKGRFTISQDNVKKTLYLQMNSLKPEDTAVYFCAVCYNLETYWGQGTQ
VTVSS
SEQ ID NO: 148 - ZHER2: 342D37(FSY)-ZHER2: 342 wt-2rs15d D54(FSY)
VDNKFNKEMRNAYWEIALLPNLNNQQKRAFIRSLYDX^{FSY}PSQSANLLAEAKKLNDAQAP
KGGGSGVDNKFNKEMRNAYWEIALLPNLNNQQKRAFIRSLYDDPSQSANLLAEAKKLND
AQAPKGGSGGGGGGSQVQLQESGGGSVQAGGSLKLTCAASGYIFNSCGMGWYRQSPGR
ERELVSRISGX^{FSY}GDTWHKESVKGRFTISQDNVKKTLYLQMNSLKPEDTAVYFCAVCYNL
ETYWGQGTQVTVSS
SEQ ID NO: 149 - ZHER2: 342 wt-2rs 15d wt
VDNKFNKEMRNAYWEIALLPNLNNQQKRAFIRSLYDDPSQSANLLAEAKKLNDAQAPK
GGSGGGGGGSQVQLQESGGGSVQAGGSLKLTCAASGYIFNSCGMGWYRQSPGRERELV
SRISGDGDTWHKESVKGRFTISQDNVKKTLYLQMNSLKPEDTAVYFCAVCYNLETYWG
QGTQVTVSS
SEQ ID NO: 150 - ZHER2: 342 D37(FSY)-2rs15d D54(FSY)
VDNKFNKEMRNAYWEIALLPNLNNQQKRAFIRSLYDX^{FSY}PSQSANLLAEAKKLNDAQA
PKGGSGGGGGGSQVQLQESGGGSVQAGGSLKLTCAASGYIFNSCGMGWYRQSPGRERE
LVSRISGX^{FSY}GDTWHKESVKGRFTISQDNVKKTLYLQMNSLKPEDTAVYFCAVCYNLET
YWGQGTQVTVSS
SEQ ID NO: 151 - ZHER2: 342 wt-ZHER2: 342 wt-2rs15d D54(FSY)
VDNKFNKEMRNAYWEIALLPNLNNQQKRAFIRSLYDDPSQSANLLAEAKKLNDAQAPKGGG
SGVDNKFNKEMRNAYWEIALLPNLNNQQKRAFIRSLYDDPSQSANLLAEAKKLNDAQAPKG
GSGGGGGGSQVQLQESGGGSVQAGGSLKLTCAASGYIFNSCGMGWYRQSPGRERELVSRIS
GX^{FSY}GDTWHKESVKGRFTISQDNVKKTLYLQMNSLKP
EDTAVYFCAVCYNLETYWGQGTQVTVSS
SEQ ID NO: 152 - ZHER2: 342 wt-ZHER2: 342 wt-7D12 wt
VDNKFNKEMRNAYWEIALLPNLNNQQKRAFIRSLYDDPSQSANLLAEAKKLNDAQAPK
GGGSGVDNKFNKEMRNAYWEIALLPNLNNQQKRAFIRSLYDDPSQSANLLAEAKKLND
AQAPKGGSGGGGGGSQVKLEESGGGSVQTGGSLRLTCAASGRTSRSYGMGWFRQAPGK
EREFVSGISWRGDSTGYADSVKGRFTISRDNAKNTVDLQMNSLKPEDTAIYYCAAAAGS
AWYGTLYEYDYWGQGTQVTVSS
SEQ ID NO: 153 - ZHER2: 342wt-ZHER2: 342 wt-7D12 Y109(FSY)
VDNKFNKEMRNAYWEIALLPNLNNQQKRAFIRSLYDDPSQSANLLAEAKKLNDAQAPKG
GGSGVDNKFNKEMRNAYWEIALLPNLNNQQKRAFIRSLYDDPSQSANLLAEAKKLNDAQ
APKGGSGGGGGGSQVKLEESGGGSVQTGGSLRLTCAASGRTSRSYGMGWFRQAPGKERE
FVSGISWRGDSTGYADSVKGRFTISRDNAKNTVDLQMNSLKPEDTAIYYCAAAAGSAWY
GTLX^{FSY}EYDYWGQGTQVTVSS
SEQ ID NO: 154 - Nanobody 7D12 (Nb_{EGFR})
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLYE YDYWGQGTQV TVSS
SEQ ID NO: 155 - Nanobody 7D12 CDR1 = RTSRSYGMG
SEQ ID NO: 156 - Nanobody 7D12 CDR2 = GISWRGDS
SEQ ID NO: 157 - Nanobody 7D12 CDR3 = AAGSAWYGTLYEYDY
SEQ ID NO: 158 = AAGSAWYGTLX^{FSY}EYDY
SEQ ID NO: 159 = AAGSAWYGTLYEYDX^{FSY}
SEQ ID NO: 160 - Nanobody 7D12
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLX^{FSY}E YDYWGQGTQV TVSS
SEQ ID NO: 161 - Nanobody 7D12
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLYEYDXFSYWGQGTQV TVSS
SEQ ID NO: 162 - Trastuzumab Fab - Light Chain:
DIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 163 - Trastuzumab Fab CDR-L1 = RASQDVNTAVA
SEQ ID NO: 164 - Trastuzumab Fab CDR-L2 = SASFLY
SEQ ID NO: 165 - Trastuzumab Fab CDR-L3 = QQHYTTPP
SEQ ID NO: 166 - Trastuzumab Fab CDR-L3 = QQHX^{FSY}TTPP
SEQ ID NO: 167 - Trastuzumab Fab CDR-L3 = QQHX^{mFSY}TTPP
SEQ ID NO: 168 - Trastuzumab Fab - Light Chain:
DIQMTQSPSSLSASVGDRVTITCRASQDVNTAVAWYQQKPGKAPKLLIYSASFLYSGVP
SRFSGSRSGTDFTLTISSLQPEDFATYYCQQHX^{FSY}TTPPTFGQGTKVEIKRTVAAPSVFIFP
PSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 169 - Trastuzumab Fab - Light Chain:
DIQMTQSPSSLSASVGDRVTITCRASQDVNTAVAWYQQKPGKAPKLLIYSASFLYSGVP
SRFSGSRSGTDFTLTISSLQPEDFATYYCQQHX^{mFSY}TTPPTFGQGTKVEIKRTVAAPSVFIF
PPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 170 - Trastuzumab Fab - Heavy Chain:
EISEVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYWGQ
GTLVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTGG
SGSAGGLNDIFEAQKIEWHE
SEQ ID NO: 171 - Trastuzumab Fab CDR-H1 = GFNIKDTYIH
SEQ ID NO: 172 - Trastuzumab Fab CDR-H2 = RIYPTNGYTRYADSVKG
SEQ ID NO: 173 - Trastuzumab Fab CDR-H3 = WGGDGFYAMDY
SEQ ID NO: 174 - NRG1b-TRX
SDKIIHLTDD SFDTDVLKAD GAILVDFWAE WCGPCKMIAP ILDEIADEYQ
GKLTVAKLNI DQNPGTAPKY GIRGIPTLLL FKNGEVAATK VGALSKGQLK
EFLDANLAGS GSGLEVLFQG PSHLVKCAEK EKTFCVNGGE CFMVKDLSNP
SRYLCKCPNE FTGDRCQNYV MX^{mFSY}SFYKHLGI EGSGSGSDYK DDDDKAAALE
SEQ ID NO: 175 - TRX
SDKIIHLTDDSFDTDVLKADGAILVDFWAEWCGPCKMIAPILDEIADEYQGKLTVAKLNI
DQNPGTAPKYGIRGIPTLLLFKNGEVAATKVGALSKGQLKEFLDANLA
SEQ ID NO: 176 - NRG1b EGF-Like Domain
SHLVKCAEKE KTFCVNGGEC FMVKDLSNPS RYLCKCPNEF TGDRCQNYVM
X^{mFSY}SFYKHLGIE
SEQ ID NO: 177 - NK035
MA QVQLQESGGG LVQPGGSLRL SCAASGKMSS RRCMAWFRQA PGKERERVAK
LLTTSGSTYL ADSVKGRFTI SQNNAKSTVY LQMNSLKPED TAMYYCAADS
FEDPTCTLVT SSGAFQYWGQ GTQVTVSS GGGGSGGGGSLPETGG

In embodiments of SEQ ID NO: 177, one amino acid selected from the group consisting of E102, D103, P104, T105, T107, L108, V109, T110, S111, S112, and G113 is replaced by meta-FSY. In embodiments of SEQ ID NO:177, one amino acid selected from the group consisting of E102, D103, P104, T105, T107, L108, V109, T110, S111, S112, and G113 is replaced by FFY.

SEQ ID NO: 178 - NK035
QVQLQESGGG LVQPGGSLRL SCAASGKMSS RRCMAWFRQA
PGKERERVAK LLTTSGSTYL ADSVKGRFTI SQNNAKSTVY
LQMNSLKPED TAMYYCAADS FEDPTCTLVT SSGAFQYWGQ
GTQVTVSS

In embodiments of SEQ ID NO: 178, one amino acid selected from the group consisting of E102, D103, P104, T105, T107, L108, V109, T110, S111, S112, and G113 is replaced by meta-FSY. In embodiments of SEQ ID NO:178, one amino acid selected from the group consisting of E102, D103, P104, T105, T107, L108, V109, T110, S111, S112, and G113 is replaced by FFY.

SEQ ID NO: 179 - NK035
QVQLQESGGG LVQPGGSLRL SCAASGKMSS RRCMAWERQA PGKERERVAK
LLTTSGSTYL ADSVKGRFTI SQNNAKSTVY LQMNSLKPED TAMYYCAADS
FEDPTCTX^{mFSY}VT SSGAFQYWGQ GTQVTVSS
SEQ ID NO: 180 ZHER2: 342 (D37FSY)
VDNKFNKEMRNAYWEIALLPNLNNQQKRAFIRSLYDX^{FSY}PSQSANLLAEAKKLNDAQA
PK
SEQ ID NO: 181 - Nanobody 7D12 CDR3 = AAGSAWYGTLX^{mFSY}EYDY
SEQ ID NO: 182 - Nanobody 7D12 CDR3 = AAGSAWYGTLYEYDX^{mFSY}
SEQ ID NO: 183 - Nanobody 7D12
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLX^{mFSY}E YDYWGQGTQV TVSS
SEQ ID NO: 184 - Nanobody 7D12
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDX^{mFSY}
WGQGTQV TVSS
SEQ ID NO: 185 - Nanobody H11-D4
QVQLVESGGGLMQAGGSLRLSCAVSGRTFSTAAMGWFRQAPGKEREFVAAIRWSGGS
AYYADSVKGRFTISRDKAKNTVYLQMNSLKYEDTAVYYCARTENVRSLLSDYATWPY
X^{FSY}YWGQGTQVTVSSK
SEQ ID NO: 186 - Nanobody H11-D4
QVQLVESGGGLMQAGGSLRLSCAVSGRTFSTAAMGWFRQAPGKEREFVAAIRWSGGS
AYYADSVKGRFTISRDKAKNTVYLQMNSLKYEDTAVYYCARTENVRSLLSDYATWPY
DX^{FSY}WGQG TQVTVSSK
SEQ ID NO: 187 - MR17-K99Y
QVQLVESGGGLVQAGGSLRLSCAASGFPVEVWRMEWYRQAPGKEREGVAAIESYGHG
TRYADSVKGRFTISRDNAKNTVYLQMNSLKPEDTAVYYCNVYDX^{FSY}GQLAYHYDYW
GQGTQVTVSAGRAGEQKLISEEDLNSAVD
SEQ ID NO: 188 - SR4
QVQLVESGGGLVQAGGSLRLSCAASGFPVYSWNMWWYRQAPGKEREWVAAIESX^{FSY}
GDSTRYADSVKGRFTISRDNAKNTVYLQMNSLKPEDTAVYYCYVWVGHTYYGQGTQ
VTVSAGRAGEQKLISEEDLNSAVD
SEQ ID NO: 189 - SR4
QVQLVESGGGLVQAGGSLRLSCAASGFPVYSWNMWWYRQAPGKEREWVAAIESHGD
X^{FSY}TRYADSVKGRFTISRDNAKNTVYLQMNSLKPEDTAVYYCYVWVGHTYYGQGQV
TVSAGRAGEQKLISEEDLNSAVD
SEQ ID NO: 190 = glycine-serine peptide linker - GGSGGGGGGS
SEQ ID NO: 191 = glycine-serine peptide linker - GGGSG
SEQ ID NO: 192 - ZHER2: 342 (D36FSY)
VDNKFNKEMR NAYWEIALLP NLNNQQKRAF IRSLYX^{FSY}DPSQ SANLLAEAKK
LNDAQAPK
SEQ ID NO: 193 - ZHER2: 342 (D36mFSY)
VDNKFNKEMR NAYWEIALLP NLNNQQKRAF IRSLYX^{mFSY}DPSQ SANLLAEAKK
LNDAQAPK
SEQ ID NO: 194 - ZHER2: 342 (D37mFSY)
VDNKFNKEMR NAYWEIALLP NLNNQQKRAF IRSLYDX^{mFSY}PSQ SANLLAEAKK
LNDAQAPK
SEQ ID NO: 195 - ZHER2: 2891 (D36FSY)
AEAKYAKEMR NAYWEIALLP NLTNQQKRAF IRKLYX^{FSY}DPSQ
SSELLSEAKKLNDSQAPK
SEQ ID NO: 196 - ZHER2: 2891 (D37FSY)
AEAKYAKEMR NAYWEIALLP NLTNQQKRAF IRKLYDX^{FSY}PSQ
SSELLSEAKKLNDSQAPK
SEQ ID NO: 197 - ZHER2: 2891 (D36mFSY)
AEAKYAKEMR NAYWEIALLP NLTNQQKRAF IRKLYX^{mFSY}DPSQ
SSELLSEAKKLNDSQAPK
SEQ ID NO: 198 - ZHER2: 2891 (D37mFSY)
AEAKYAKEMR NAYWEIALLP NLTNQQKRAF IRKLYDX^{mFSY}PSQ
SSELLSEAKKLNDSQAPK
SEQ ID NO: 199 - Nanobody 7D12 (Nb_{EGFR})
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLYE YDYWGX^{FSY}GTQV TVSS
SEQ ID NO: 200 = DPASPAYGDX^{FSY} = CDR3, mNb6
SEQ ID NO: 201 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYGDX^{FSY}WG QGTQVTVSS
SEQ ID NO: 202 = DPASPAYX^{FSY}DY = CDR3, mNb6
SEQ ID NO: 203 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYX^{FSY}DYWG QGTQVTVSS
SEQ ID NO: 204 = DPASPAYX^{FFY}DY = CDR3, mNb6
SEQ ID NO: 205 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYX^{FFY}DYWG QGTQVTVSS
SEQ ID NO: 206 = DPASPX^{FSY}YGDY = CDR3, mNb6
SEQ ID NO: 207 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPX^{FSY}YGDYWG QGTQVTVSS
SEQ ID NO: 208 = DPASPX^{FFY}YGDY = CDR3, mNb6
SEQ ID NO: 209 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPX^{FFY}YGDYWG QGTQVTVSS
SEQ ID NO: 210 = DPAX^{FSY}PAYGDY = CDR3, mNb6
SEQ ID NO: 211 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
AX^{FSY}PAYGDYWG QGTQVTVSS
SEQ ID NO: 212 = DPAX^{FFY}PAYGDY = CDR3, mNb6
SEQ ID NO: 213 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
AX^{FFY}PAYGDYWG QGTQVTVSS
SEQ ID NO: 214 - A1
QVQLVQSGGG LVHPGGSLRL SCAASGIDLS LYRMRWYRQA PGKERDLVAL
ITDDGTSYYE DSVKGRFTIT RDNPSNKVFL QMNSLKPEDT AVYYCNAETP
LSPVNYWGQG TQVTVSS
SEQ ID NO: 215 - A1 CDR1 = GIDLSLYR
SEQ ID NO: 216 - A1 CDR2 = ITDDGTS
SEQ ID NO: 217 - A1 CDR3 = NAETPLSPVNY
SEQ ID NO: 218 - A1 CDR1 = X^{FSY}IDLSLYR
SEQ ID NO: 219 - A1 CDR1 = GIX^{FSY}LSLYR
SEQ ID NO: 220 - A1 CDR1 = GIDLX^{FSY}LYR
SEQ ID NO: 221 - A1 CDR1 = GIDLSX^{FSY}YR
SEQ ID NO: 222 - A1 CDR1 = GIDLSLYX^{FSY}
SEQ ID NO: 223 - A1 CDR3 = NAEX^{FSY}PLSPVNY
SEQ ID NO: 224 - A1 CDR3 = NAETX^{FSY}LSPVNY
SEQ ID NO: 225 - A1 CDR3 = NAETPX^{FSY}SPVNY
SEQ ID NO: 226 - A1 CDR3 = NAETPLSX^{FSY}VNY
SEQ ID NO: 227 - A1 G26(FSY)
QVQLVQSGGGLVHPGGSLRLSCAASX^{FSY}IDLSLYRMRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 228 - A1 D28(FSY)
QVQLVQSGGGLVHPGGSLRLSCAASGIX^{FSY}LSLYRMRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 229 - A1 S30(FSY)
QVQLVQSGGGLVHPGGSLRLSCAASGIDLX^{FSY}LYRMRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 230 - A1 L31(FSY)
QVQLVQSGGGLVHPGGSLRLSCAASGIDLSX^{FSY}YRMRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 231 - A1 R33(FSY)
QVQLVQSGGGLVHPGGSLRLSCAASGIDLSLYX^{FSY}MRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 232 - A1 151(FSY)
QVQLVQSGGGLVHPGGSLRLSCAASGIDLSLYRMRWYRQAPGKERDLVALX^{FSY}TDDGTSYYEDS
VKGRFTITRDNPSNKVFLQMNSLKPEDTAVYYCNAETPLSPVNYWGQGTQVTVSS
SEQ ID NO: 233 - A1 D53(FSY)
QVQLVQSGGGLVHPGGSLRLSCAASGIDLSLYRMRWYRQAPGKERDLVALITX^{FSY}DGTSYYEDS
VKGRFTITRDNPSNKVFLQMNSLKPEDTAVYYCNAETPLSPVNYWGQGTQVTVSS
SEQ ID NO: 234 - A1 D54(FSY)
QVQLVQSGGGLVHPGGSLRLSCAASGIDLSLYRMRWYRQAPGKERDLVALITDX^{FSY}GTSYYEDS
VKGRFTITRDNPSNKVFLQMNSLKPEDTAVYYCNAETPLSPVNYWGQGTQVTVSS
SEQ ID NO: 235 - A1 T99(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAEX^{FSY}PLSPVNYWGQGTOVTVSS
SEQ ID NO: 236 - A1 P100(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAETX^{FSY}LSPVNYWGQGTQVTVSS
SEQ ID NO: 237 - A1 L101(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAETPX^{FSY}SPVNYWGQGTQVTVSS
SEQ ID NO: 238 - A1 P103(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAETPLSX^{FSY}VNYWGQGTQVTVSS
SEQ ID NO: 239 - C6
QVQLVQSGGG LVQAGGSLRL SCAPSGSIFG IRTMDWYRQA PGKERELVAR
ITMDGRVFHA DSVKGRFSGS RDGASNAVYL QMNSLKPDDT AVYYCRYSGL
TSREDYWGPG TQVTVSS
SEQ ID NO: 240 - C6 CDR1 = GSIFGIRT
SEQ ID NO: 241 - C6 CDR2 = ITMDGRV
SEQ ID NO: 242 - C6 CDR3 = RYSGLTSREDY
SEQ ID NO: 243 - C6 CDR1 = GX^{FSY}IFGIRT
SEQ ID NO: 244 - C6 CDR1 = GSIX^{FSY}GIRT
SEQ ID NO: 245 - C6 CDR1 = GSIFGX^{FSY}RT
SEQ ID NO: 246 - C6 CDR1 = GSIFGIX^{FSY}T
SEQ ID NO: 247 - C6 CDR2 = IX^{FSY}MDGRV
SEQ ID NO: 248 - C6 CDR2 = ITX^{FSY}DGRV
SEQ ID NO: 249 - C6 CDR2 = ITMX^{FSY}GRV
SEQ ID NO: 250 - C6 CDR2 = ITMDX^{FSY}RV
SEQ ID NO: 251 - C6 CDR3 = X^{FSY}YSGLTSREDY
SEQ ID NO: 252 - C6 CDR3 = RYSGLX^{FSY}SREDY
SEQ ID NO: 253 - C6 CDR3 = RYSGLTX^{FSY}REDY
SEQ ID NO: 254 - C6 CDR3 = RYSGLTSREX^{FSY}Y
SEQ ID NO: 255 - C6 S27(FSY)
QVQLVQSGGGLVQAGGSLRLSCAPSGX^{FSY}IFGIRTMDWYRQAPGKERELVARITMDGRVFHADS
VKGRFSGSRDGASNAVYLQMNSLKPDDTAVYYCRYSGLTSREDYWGPGTQVTVSS
SEQ ID NO: 256 - C6 F29(FSY)
QVQLVQSGGGLVQAGGSLRLSCAPSGSIX^{FSY}GIRTMDWYRQAPGKERELVARITMDGRVFHADS
VKGRFSGSRDGASNAVYLQMNSLKPDDTAVYYCRYSGLTSREDYWGPGTQVTVSS
SEQ ID NO: 257 - C6 I31(FSY)
QVQLVQSGGGLVQAGGSLRLSCAPSGSIFGX^{FSY}RTMDWYRQAPGKERELVARITMDGRVFHADS
VKGRFSGSRDGASNAVYLQMNSLKPDDTAVYYCRYSGLTSREDYWGPGTQVTVSS
SEQ ID NO: 258 - C6 R32(FSY)
QVQLVQSGGGLVQAGGSLRLSCAPSGSIFGIX^{FSY}TMDWYRQAPGKERELVARITMDGRVFHADS
VKGRFSGSRDGASNAVYLQMNSLKPDDTAVYYCRYSGLTSREDYWGPGTQVTVSS
SEQ ID NO: 259 - C6 T52(FSY)
QVQLVQSGGGLVQAGGSLRLSCAPSGSIFGIRTMDWYRQAPGKERELVARIX^{FSY}MDGRVFHADS
VKGRFSGSRDGASNAVYLQMNSLKPDDTAVYYCRYSGLTSREDYWGPGTQVTVSS
SEQ ID NO: 260 - C6 M53(FSY)
QVQLVQSGGGLVQAGGSLRLSCAPSGSIFGIRTMDWYRQAPGKERELVARITX^{FSY}DGRVFHADS
VKGRFSGSRDGASNAVYLQMNSLKPDDTAVYYCRYSGLTSREDYWGPGTQVTVSS
SEQ ID NO: 261 - C6 D54(FSY)
QVQLVQSGGGLVQAGGSLRLSCAPSGSIFGIRTMDWYRQAPGKERELVARITMX^{FSY}GRVFHADS
VKGRFSGSRDGASNAVYLQMNSLKPDDTAVYYCRYSGLTSREDYWGPGTQVTVSS
SEQ ID NO: 262 - C6 G55(FSY)
QVQLVQSGGGLVQAGGSLRLSCAPSGSIFGIRTMDWYRQAPGKERELVARITMDX^{FSY}RVFHADS
VKGRFSGSRDGASNAVYLQMNSLKPDDTAVYYCRYSGLTSREDYWGPGTQVTVSS
SEQ ID NO: 263 - C6 R96(FSY)
QVQLVQSGGGLVQAGGSLRLSCAPSGSIFGIRTMDWYRQAPGKERELVARITMDGRVFHADSVK
GRFSGSRDGASNAVYLQMNSLKPDDTAVYYCX^{FSY}YSGLTSREDYWGPGTQVTVSS
SEQ ID NO: 264 - C6 T101(FSY)
QVQLVQSGGGLVQAGGSLRLSCAPSGSIFGIRTMDWYRQAPGKERELVARITMDGRVFHADSVK
GRFSGSRDGASNAVYLQMNSLKPDDTAVYYCRYSGLX^{FSY}SREDYWGPGTQVTVSS
SEQ ID NO: 265 - C6 S102(FSY)
QVQLVQSGGGLVQAGGSLRLSCAPSGSIFGIRTMDWYRQAPGKERELVARITMDGRVFHADSVK
GRFSGSRDGASNAVYLQMNSLKPDDTAVYYCRYSGLTX^{FSY}REDYWGPGTQVTVSS
SEQ ID NO: 266 - C6 D105(FSY)
QVQLVQSGGGLVQAGGSLRLSCAPSGSIFGIRTMDWYRQAPGKERELVARITMDGRVFHADSVK
GRFSGSRDGASNAVYLQMNSLKPDDTAVYYCRYSGLTSREX^{FSY}YWGPGTQVTVSS
SEQ ID NO: 60 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYGDYWG QGTQVTVSS
SEQ ID NO: 61 = GYIFGRNAMG = CDR1, mNb6 nanobody
SEQ ID NO: 62 = GITRRGSITY = CDR2, mNb6
SEQ ID NO: 63 = DPASPAYGDY = CDR3, mNb6
SEQ ID NO: 64 = DPASPAYGDX^{FFY} = CDR3, mNb6
SEQ ID NO: 65 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYGDX^{FFY}WG QGTQVTVSS
SEQ ID NO: 66 - Nanobody 2rs15d (Nb_{Her2})
QVQLQESGGG SVQAGGSLKL TCAASGYIFN SCGMGWYRQS PGRERELVSR
ISGDGDTWHK ESVKGRFTIS QDNVKKTLYL QMNSLKPEDT AVYFCAVCYN
LETYWGQGTQ VTVSS
SEQ ID NO: 67 - Nanobody 2rs15d CDR1 = GYIFNSCG
SEQ ID NO: 68 - Nanobody 2rs15d CDR2 = RISGDGD
SEQ ID NO: 69 - Nanobody 2rs15d CDR3 = AVCYNLETY
SEQ ID NO: 70 - Nanobody 2rs15d CDR2 = RISGX^{FSY}GD
SEQ ID NO: 71 - Nanobody 2rs15d CDR3 = AVCYNLX^{FSY}TY
SEQ ID NO: 72 - Nanobody 2rs15d (Nb_{Her2}) - 2rs15D(D54FSY)
QVQLQESGGG SVQAGGSLKL TCAASGYIFN SCGMGWYRQS PGRERELVSR
ISGX^{FSY}GDTWHK ESVKGRFTIS QDNVKKTLYL QMNSLKPEDT AVYFCAVCYN
LETYWGQGTQ VTVSS
SEQ ID NO: 73 - Nanobody 2rs15d (Nb_{Her2}) - 2rs15D(E102FSY)
QVQLQESGGG SVQAGGSLKL TCAASGYIFN SCGMGWYRQS PGRERELVSR
ISGDGDTWHK ESVKGRFTIS QDNVKKTLYL QMNSLKPEDT AVYFCAVCYN
LX^{FSY}TYWGQGTQ VTVSS
SEQ ID NO: 74 - Nanobody C21
EVQLVESGGE LVQAGGSLRL SCAASGLTFS SYNMGWFRRA PGKEREFVAS
ITWSGRDTFY ADSVKGRFTI SRDNAKNTVY LQMSSLKPED TAVYYCAANP
WPVAAPRSGT YWGQGTQVTV SS
SEQ ID NO: 75 - Nanobody C21 CDR1 = GLTFSSYN
SEQ ID NO: 76 - Nanobody C21 CDR2 = ITWSGRDT
SEQ ID NO: 77 - Nanobody C21 CDR3 = AANPWPVAAPRSGTY
SEQ ID NO: 78 - Nanobody C21 CDR1 = GLTFSX^{FSY}YN
SEQ ID NO: 79 - Nanobody C21
EVQLVESGGE LVQAGGSLRL SCAASGLTFS X^{FSY}YNMGWFRRA PGKEREFVAS
ITWSGRDTFY ADSVKGRFTI SRDNAKNTVY LQMSSLKPED TAVYYCAANP
WPVAAPRSGT YWGQGTQVTV SS
SEQ ID NO: 80 = Nanobody NB13
QVQLQESGGG SVQTGGSLRL SCAASGYTAS FSWIGYFRQA PGKEREGVAV
INVGVGSTYY ADSVKGRFTI SRDNTENTIS LEMNSLKPED TGLYYCAGSL
RWSRPPNPIS EDAYNYWGQG TQVTVSS
SEQ ID NO: 81 = Nanobody NB13 CDR1 = GYTASFSWI
SEQ ID NO: 82 = Nanobody NB13 CDR2 = VINVGVGST
SEQ ID NO: 83 = Nanobody NB13 CDR3 = AGSLRWSRPPNPISEDAYNY
SEQ ID NO: 84 = Nanobody NB13 CDR2 = VINVX^{FSY}VGST
SEQ ID NO: 85 = Nanobody NB13 CDR2 = VINVGVGX^{FSY}T
SEQ ID NO: 86 = Nanobody NB13 CDR1 = GYTASFX^{mFSY}WI
SEQ ID NO: 87 = Nanobody NB13 CDR2 = VINVX^{mFSY}VGST
SEQ ID NO: 88 = Nanobody NB13
QVQLQESGGG SVQTGGSLRL SCAASGYTAS FSWIGYFRQA PGKEREGVAV
INVX^{FSY}VGSTYY ADSVKGRFTI SRDNTENTIS LEMNSLKPED TGLYYCAGSL
RWSRPPNPIS EDAYNYWGQG TQVTVSS
SEQ ID NO: 89 = Nanobody NB13
QVQLQESGGG SVQTGGSLRL SCAASGYTAS FSWIGYFRQA PGKEREGVAV
INVGVGX^{FSY}TYY ADSVKGRFTI SRDNTENTIS LEMNSLKPED TGLYYCAGSL
RWSRPPNPIS EDAYNYWGQG TQVTVSS
SEQ ID NO: 90 = Nanobody NB13
QVQLQESGGG SVQTGGSLRL SCAASGYTAS FX^{mFSY}WIGYFRQA PGKEREGVAV
INVGVGSTYY ADSVKGRFTI SRDNTENTIS LEMNSLKPED TGLYYCAGSL
RWSRPPNPIS EDAYNYWGQG TQVTVSS
SEQ ID NO: 91 = Nanobody NB13
QVQLQESGGG SVQTGGSLRL SCAASGYTAS FSWIGYFRQA PGKEREGVAV
INVX^{mFSY}VGSTYY ADSVKGRFTI SRDNTENTIS LEMNSLKPED TGLYYCAGSL
RWSRPPNPIS EDAYNYWGQG TQVTVSS
SEQ ID NO: 92 - Nanobody NB17B05 (alternatively “MS83”)
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TRGSSDYWGQ GTLVTVSSAA AEQKLISEED LNGAA
SEQ ID NO: 93 = LNAMA
SEQ ID NO: 94 = GIFGVGSTRYADSVKG
SEQ ID NO: 95 = SSVTRGSSDY
SEQ ID NO: 96 = GIFGVGSTRX^{FSY}ADSVKG
SEQ ID NO: 97 = GIFGVGSTRYX^{FSY}DSVKG
SEQ ID NO: 98 = GIFGVGSTRYAX^{FSY}SVKG
SEQ ID NO: 99 = GIFGVGSTRYADX^{FSY}VKG
SEQ ID NO: 100 = GIFGVGSTRYADSX^{FSY}KG
SEQ ID NO: 101 = GIFGVGSTRYADSVX^{FSY}G
SEQ ID NO: 102 = GIFGVGSTRYADSVKX^{FSY}
SEQ ID NO: 103 = SSVTX^{FSY}GSSDY
SEQ ID NO: 104 = SSVTRX^{FSY}SSDY
SEQ ID NO: 105 = GIFGVGSX^{mFSY}RYADSVKG
SEQ ID NO: 106 = GIFGVGSTX^{mFSY}YADSVKG
SEQ ID NO: 107 = GIFGVGSTRX^{mFSY}ADSVKG
SEQ ID NO: 108 = GIFGVGSTRYX^{mFSY}DSVKG
SEQ ID NO: 109 = GIFGVGSTRYAX^{mFSY}SVKG
SEQ ID NO: 110 = GIFGVGSTRYADX^{mFSY}VKG
SEQ ID NO: 111 = GIFGVGSTRYADSX^{mFSY}KG
SEQ ID NO: 112 = GIFGVGSTRYADSVX^{mFSY}G
SEQ ID NO: 113 = GIFGVGSTRYADSVKX^{mFSY}
SEQ ID NO: 114 = SSVTX^{mFSY}GSSDY
SEQ ID NO: 115 = SSVTRX^{mFSY}SSDY
SEQ ID NO: 116 - Nanobody NB17B05
EVQLVESGGG LVQPGGSLRL SCAASGSIGG LNAMAWYRQA PGKERELVAG
IFGVGSTRX^{FSY}A DSVKGRFTIS RDNSKNTVYL QMNSLRSEDT AVYYCAMSSV
TRGSSDYWGQ GTLVTVSSAA AEQKLISEED LNGAA
SEQ ID NO: 117- Nanobody NB17B05
EVQLVESGGG LVQPGGSLRL SCAASGSIGG LNAMAWYRQA PGKERELVAG
IFGVGSTRYX^{FSY} DSVKGRFTIS RDNSKNTVYL QMNSLRSEDT AVYYCAMSSV
TRGSSDYWGQ GTLVTVSSAA AEQKLISEED LNGAA
SEQ ID NO: 118 - Nanobody NB17B05
EVQLVESGGG LVQPGGSLRL SCAASGSIGG LNAMAWYRQA PGKERELVAG
IFGVGSTRYA X^{FSY}SVKGRFTIS RDNSKNTVYL QMNSLRSEDT AVYYCAMSSV
TRGSSDYWGQ GTLVTVSSAA AEQKLISEED LNGAA
SEQ ID NO: 119 - Nanobody NB17B05
EVQLVESGGG LVQPGGSLRL SCAASGSIGG LNAMAWYRQA PGKERELVAG
IFGVGSTRYA DX^{FSY}VKGRFTIS RDNSKNTVYL QMNSLRSEDT AVYYCAMSSV
TRGSSDYWGQ GTLVTVSSAA AEQKLISEED LNGAA
SEQ ID NO: 120 - Nanobody NB17B05
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TRX^{FSY}SSDYWGQ GTLVTVSSAA AEQKLISEED LNGAA
SEQ ID NO: 121 - Nanobody NB17B05
EVQLVESGGG LVQPGGSLRL SCAASGSIGG LNAMAWYRQA PGKERELVAG
IFGVGSX^{mFSY}RYA DSVKGRFTIS RDNSKNTVYL QMNSLRSEDT AVYYCAMSSV
TRGSSDYWGQ GTLVTVSSAA AEQKLISEED LNGAA
SEQ ID NO: 122 - Nanobody NB17B05
EVQLVESGGG LVQPGGSLRL SCAASGSIGG LNAMAWYRQA PGKERELVAG
IFGVGSTX^{mFSY}YA DSVKGRFTIS RDNSKNTVYL QMNSLRSEDT AVYYCAMSSV
TRGSSDYWGQ GTLVTVSSAA AEQKLISEED LNGAA
SEQ ID NO: 123 - Nanobody NB17B05
EVQLVESGGG LVQPGGSLRL SCAASGSIGG LNAMAWYRQA PGKERELVAG
IFGVGSTRX^{mFSY}A DSVKGRFTIS RDNSKNTVYL QMNSLRSEDT AVYYCAMSSV
TRGSSDYWGQ GTLVTVSSAA AEQKLISEED LNGAA
SEQ ID NO: 124 - Nanobody NB17B05
EVQLVESGGG LVQPGGSLRL SCAASGSIGG LNAMAWYRQA PGKERELVAG
IFGVGSTRYA DSVX^{mFSY}GRFTIS RDNSKNTVYL QMNSLRSEDT AVYYCAMSSV
TRGSSDYWGQ GTLVTVSSAA AEQKLISEED LNGAA
SEQ ID NO: 125 - Nanobody NB17B05
EVQLVESGGG LVQPGGSLRL SCAASGSIGG LNAMAWYRQA PGKERELVAG
IFGVGSTRYA DSVKX^{mFSY}RFTIS RDNSKNTVYL QMNSLRSEDT AVYYCAMSSV
TRGSSDYWGQ GTLVTVSSAA AEQKLISEED LNGAA
SEQ ID NO: 126 - Nanobody NB17B05
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TX^{mFSY}GSSDYWGQ GTLVTVSSAA AEQKLISEED LNGAA
SEQ ID NO: 127 - Nanobody NB17B05
EVQLVESGGG LVQPGGSLRL SCAASGSIGG LNAMAWYRQA PGKERELVAG
IFGVGSTRYA DSVKGRFTIS RDNSKNTVYL QMNSLRSEDT AVYYCAMSSV
TRX^{mFSY}SSDYWGQ GTLVTVSSAA AEQKLISEED LNGAA
SEQ ID NO: 128 - MKYLLPTAAA GLLLLAAQPA MAMA = optionally present at
the N-terminus of nanobody NB17B05
SEQ ID NO: 129 - MS211
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SS
In the sequences below, the glycine-serine peptide linker is underlined.
SEQ ID NO: 130 - Dimer10-60FSY (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGGSGGGGSEVQLVESGGGLVQPGGSLRLSCAASGSIGGLNAMAWYRQAPGKEREL
VAGIFGVGSTRYX^{FSY}DSVKGRFTISRDNSKNTVYLQMNSLRSEDTAVYYCRMSSVTRGSS
DYWGQGTLVTVSSAAAEQKLISEEDLNGAA In embodiments, the dimer further comprises
SEQ ID NO: 128 at the N-terminus
SEQ ID NO: 131 - Dimer12-60FSY - (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGSGGGSGGGSEVQLVESGGGLVQPGGSLRLSCAASGSIGGLNAMAWYRQAPGKER
ELVAGIFGVGSTRYX^{FSY}DSVKGRFTISRDNSKNTVYLQMNSLRSEDTAVYYCRMSSVTRGS
SDYWGQGTLVTVSSAAAEQKLISEEDLNGAA In embodiments, the dimer further comprises
SEQ ID NO: 128 at the N-terminus
SEQ ID NO: 132 - Dimer15-60FSY - (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGGSGGGGSGGGGSEVQLVESGGGLVQPGGSLRLSCAASGSIGGLNAMAWYRQAPG
KERELVAGIFGVGSTRYX^{FSY}DSVKGRFTISRDNSKNTVYLQMNSLRSEDTAVYYCRMSSV
TRGSSDYWGQGTLVTVSSAAAEQKLIS EEDLNGAA In embodiments, the dimer further
comprises SEQ ID NO: 128 at the N-terminus
SEQ ID NO: 133 - Dimer20-60FSY - (MS211-NB17B05)
EVQLVESGGGLVQPGGSLRLSCAASGTLFKINAMGWYRQAPGKRRELVALITSSDTTDYA
DSVKGRFTISRDNSWNTVYLQMNSLRPEDTAVYYCHSDHYSLGVPEKRVILYGQGTLVTV
SSGGGGSGGGGSGGGGSGGGGSEVQLVESGGGLVQPGGSLRLSCAASGSIGGLNAMAWY
RQAPGKERELVAGIFGVGSTRYX^{FSY}DSVKGRFTISRDNSKNTVYLQMNSLRSEDTAVYYC
RMSSVTRGSSDYWGQGTLVTVSSAAA EQKLISEEDLNGAA In embodiments, the dimer
further comprises SEQ ID NO: 128 at the N-terminus
SEQ ID NO: 134 - 7D12 wt-2rs15D wt
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYW
GQGTQVTVSSGGSGGGGGGSQVQLQESGGGSVQAGGSLKLTCAASGYIFNSCGMGWY
RQSPGRERELVSRISGDGDTWHKESVKGRFTISQDNVKKTLYLQM NSLKPEDTAVYFC
AVCYNLETYWGQGTQVTVSS
SEQ ID NO: 135 - 7D12 wt-2rs15D D54(FSY)
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYW
GQGTQVTVSSGGSGGGGGGSQVQLQESGGGSVQAGGSLKLTCAASGYIFNSCGMGWY
RQSPGRERELVSRISGX^{FSY}GDTWHKESVKGRFTISQDNVKKTLYLQMNSLKPEDTAVYF
CAVCYNLETYWGQGTQVTVSS
SEQ ID NO: 136 - 7D12 Y109(FSY)-2rs15D D54(FSY)
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLX^{FSY}EYDY
WGQGTQVTVSSGGSGGGGGGSQVQLQESGGGSVQAGGSLKLTCAASGYIFNSCGMG
WYRQSPGRERELVSRISGX^{FSY}GDTWHKESVKGRFTISQDNVKKTLYLQM
NSLKPEDTAVYFC AVCYNLETYWGQGTQVTVSS
SEQ ID NO: 137 - ZHER2: 2891
AEAKYAKEMR NAYWEIALLP NLTNQQKRAF IRKLYDDPSQ SSELLSEAKKLNDSQAPK
SEQ ID NO: 138 - ZHER2: 342
VDNKFNKEMR NAYWEIALLP NLNNQQKRAF IRSLYDDPSQ SANLLAEAKK
LNDAQAPK
SEQ ID NO: 139 - F57
EVQLVESGGGLVQAGGSLRLSCAASGITFSINTMGWYRQAPGKQRELVALISSIGDTYY
ADSVKGRFTISRDNAKNTVYLQMNSLKPEDTAVYYCKRFRTAAQGTDYWGQGTQVTV
SS
SEQ ID NO: 140 - 7D12 wt-ZHER2: 2891 wt
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLYEYDYWGQG
TQVTVSSGGSGGGGGGSAEAKYAKEMRNAYWEIALLPNLTNQQKRAFIRKLYDDPSQSS
ELLSEAKKLNDSQAPK
SEQ ID NO: 141 - 7D12 Y109(FSY)-ZHER2: 2891
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLX^{FSY}EYDYW
GQGTQVTVSSGGSGGGGGGSAEAKYAKEMRNAYWEIALLPNLTNQQKRAFIRKLYDDP
SQSSELLSEAKKLNDSQAPK
SEQ ID NO: 142 - 7D12 wt-ZHER2: 342 wt
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLYEYDYWGQ
GTQVTVSSGGSGGGGGGSVDNKFNKEMRNAYWEIALLPNLNNQQKRAFIRSLYDDPSQS
ANLLAEAKKLNDAQAPK
SEQ ID NO: 143 - 7D12 Y109(FSY)-ZHER2: 342 wt
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLX^{FSY}EYDYW
GQGTQVTVSSGGSGGGGGGSVDNKFNKEMRNAYWEIALLPNLNNQQKRAFIRSLYDDP
SQSANLLAEAKKLNDAQAPK
SEQ ID NO: 144 - 7D12 Y109(FSY)-ZHER2: 342 D37
QVKLEESGGGSVQTGGSLRLTCAASGRTSRSYGMGWFRQAPGKEREFVSGISWRGDSTG
YADSVKGRFTISRDNAKNTVDLQMNSLKPEDTAIYYCAAAAGSAWYGTLX^{FSY}EYDYW
GQGTQVTVSSGGSGGGGGGSVDNKFNKEMRNAYWEIALLPNLNNQQKRAFIRSLYDX^{FSY}
PSQSANLLAEAKKLNDAQAPK
SEQ ID NO: 145 - 7D12 wt- 5F7
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYW
GQGTQVTVSSGGSGGGGGGSEVQLVESGGGLVQAGGSLRLSCAASGITFSINTMGWYR
QAPGKQRELVALISSIGDTYYADSVKGRFTISRDNAKNTVYLQMNSLKPEDTAVYYCK
RFRTAAQGTDYWGQGTQVTVSS
SEQ ID NO: 146 - 7D12 Y109(FSY)-5F7
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLX^{FSY}EYDY
WGQGTQVTVSSGGSGGGGGGSEVQLVESGGGLVQAGGSLRLSCAASGITFSINTMGW
YRQAPGKQRELVALISSIGDTYYADSVKGRFTISRDNAKNTVYLQMNSLKPEDTAVYY
CKRFRTAAQGTDYWGQGTQVTVSS
SEQ ID NO: 147 - ZHER2: 342 wt-ZHER2: 342 wt-2rs15d wt
VDNKFNKEMRNAYWEIALLPNLNNQQKRAFIRSLYDDPSQSANLLAEAKKLNDAQAPKV
DNKFNKEMRNAYWEIALLPNLNNQQKRAFIRSLYDDPSQSANLLAEAKKLNDAQAPKGG
SGGGGGGSQVQLQESGGGSVQAGGSLKLTCAASGYIFNSCGMGWYRQSPGRERELVSRIS
GDGDTWHKESVKGRFTISQDNVKKTLYLQMNSLKPEDTAVYFCAVCYNLETYWGQGTQ
VTVSS
SEQ ID NO: 148 - ZHER2: 342D37(FSY)-ZHER2: 342 wt-2rs15d D54(FSY)
VDNKFNKEMRNAYWEIALLPNLNNQQKRAFIRSLYDX^{FSY}PSQSANLLAEAKKLNDAQAP
KGGGSGVDNKFNKEMRNAYWEIALLPNLNNQQKRAFIRSLYDDPSQSANLLAEAKKLND
AQAPKGGSGGGGGGSQVQLQESGGGSVQAGGSLKLTCAASGYIFNSCGMGWYRQSPGR
ERELVSRISGX^{FSY}GDTWHKESVKGRFTISQDNVKKTLYLQMNSLKPEDTAVYFCAVCYNL
ETYWGQGTQVTVSS
SEQ ID NO: 149 - ZHER2: 342 wt-2rs15d wt
VDNKFNKEMRNAYWEIALLPNLNNQQKRAFIRSLYDDPSQSANLLAEAKKLNDAQAPK
GGSGGGGGGSQVQLQESGGGSVQAGGSLKLTCAASGYIFNSCGMGWYRQSPGRERELV
SRISGDGDTWHKESVKGRFTISQDNVKKTLYLQMNSLKPEDTAVYFCAVCYNLETYWG
QGTQVTVSS
SEQ ID NO: 150 - ZHER2: 342 D37(FSY)-2rs15d D54(FSY)
VDNKFNKEMRNAYWEIALLPNLNNQQKRAFIRSLYDX^{FSY}PSQSANLLAEAKKLNDAQA
PKGGSGGGGGGSQVQLQESGGGSVQAGGSLKLTCAASGYIFNSCGMGWYRQSPGRERE
LVSRISGX^{FSY}GDTWHKESVKGRFTISQDNVKKTLYLQMNSLKPEDTAVYFCAVCYNLET
YWGQGTQVTVSS
SEQ ID NO: 151 - ZHER2: 342 wt-ZHER2: 342 wt-2rs15d D54(FSY)
VDNKFNKEMRNAYWEIALLPNLNNQQKRAFIRSLYDDPSQSANLLAEAKKLNDAQAPKGGG
SGVDNKFNKEMRNAYWEIALLPNLNNQQKRAFIRSLYDDPSQSANLLAEAKKLNDAQAPKG
GSGGGGGGSQVQLQESGGGSVQAGGSLKLTCAASGYIFNSCGMGWYRQSPGRERELVSRIS
GX^{FSY}GDTWHKESVKGRFTISQDNVKKTLYLQMNSLKP
EDTAVYFCAVCYNLETYWGQGTQVTVSS
SEQ ID NO: 152 - ZHER2: 342 wt-ZHER2: 342 wt-7D12 wt
VDNKFNKEMRNAYWEIALLPNLNNQQKRAFIRSLYDDPSQSANLLAEAKKLNDAQAPK
GGGSGVDNKFNKEMRNAYWEIALLPNLNNQQKRAFIRSLYDDPSQSANLLAEAKKLND
AQAPKGGSGGGGGGSQVKLEESGGGSVQTGGSLRLTCAASGRTSRSYGMGWFRQAPGK
EREFVSGISWRGDSTGYADSVKGRFTISRDNAKNTVDLQMNSLKPEDTAIYYCAAAAGS
AWYGTLYEYDYWGQGTQVTVSS
SEQ ID NO: 153 - ZHER2: 342wt-ZHER2: 342 wt-7D12 Y109(FSY)
VDNKFNKEMRNAYWEIALLPNLNNQQKRAFIRSLYDDPSQSANLLAEAKKLNDAQAPKG
GGSGVDNKFNKEMRNAYWEIALLPNLNNQQKRAFIRSLYDDPSQSANLLAEAKKLNDAQ
APKGGSGGGGGGSQVKLEESGGGSVQTGGSLRLTCAASGRTSRSYGMGWFRQAPGKERE
FVSGISWRGDSTGYADSVKGRFTISRDNAKNTVDLQMNSLKPEDTAIYYCAAAAGSAWY
GTLX^{FSY}EYDYWGQGTQVTVSS
SEQ ID NO: 154 - Nanobody 7D12 (Nb_{EGFR})
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLYE YDYWGQGTQV TVSS
SEQ ID NO: 155 - Nanobody 7D12 CDR1 = RTSRSYGMG
SEQ ID NO: 156 - Nanobody 7D12 CDR2 = GISWRGDS
SEQ ID NO: 157 - Nanobody 7D12 CDR3 = AAGSAWYGTLYEYDY
SEQ ID NO: 158 = AAGSAWYGTLX^{FSY}EYDY
SEQ ID NO: 159 = AAGSAWYGTLYEYDX^{FSY}
SEQ ID NO: 160 - Nanobody 7D12
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLX^{FSY}E YDYWGQGTQV TVSS
SEQ ID NO: 161 - Nanobody 7D12
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLYEYDX^{FSY}WGQGTQV TVSS
SEQ ID NO: 162 - Trastuzumab Fab - Light Chain:
DIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 163 - Trastuzumab Fab CDR-LI = RASQDVNTAVA
SEQ ID NO: 164 - Trastuzumab Fab CDR-L2 = SASFLY
SEQ ID NO: 165 - Trastuzumab Fab CDR-L3 = QQHYTTPP
SEQ ID NO: 166 - Trastuzumab Fab CDR-L3 = QQHX^{FSY}TTPP
SEQ ID NO: 167 - Trastuzumab Fab CDR-L3 = QQHX^{mFSY}TTPP
SEQ ID NO: 168 - Trastuzumab Fab - Light Chain:
DIQMTQSPSSLSASVGDRVTITCRASQDVNTAVAWYQQKPGKAPKLLIYSASFLYSGVP
SRFSGSRSGTDFTLTISSLQPEDFATYYCQQHX^{FSY}TTPPTFGQGTKVEIKRTVAAPSVFIFP
PSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 169 - Trastuzumab Fab - Light Chain:
DIQMTQSPSSLSASVGDRVTITCRASQDVNTAVAWYQQKPGKAPKLLIYSASFLYSGVP
SRFSGSRSGTDFTLTISSLQPEDFATYYCQQHX^{mFSY}TTPPTFGQGTKVEIKRTVAAPSVFIF
PPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 170 - Trastuzumab Fab - Heavy Chain:
EISEVQLVESGGGLVQPGGSLRLSCAASGFNIKDTYIHWVRQAPGKGLEWVARIYPING
YTRYADSVKGRFTISADTSKNTAYLQMNSLRAEDTAVYYCSRWGGDGFYAMDYWGQ
GTLVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTGG
SGSAGGLNDIFEAQKIEWHE
SEQ ID NO: 171 - Trastuzumab Fab CDR-H1 = GFNIKDTYIH
SEQ ID NO: 172 - Trastuzumab Fab CDR-H2 = RIYPTNGYTRYADSVKG
SEQ ID NO: 173 - Trastuzumab Fab CDR-H3 = WGGDGFYAMDY
SEQ ID NO: 174 - NRG1b-TRX
SDKIIHLTDD SFDTDVLKAD GAILVDFWAE WCGPCKMIAP ILDEIADEYQ
GKLTVAKLNI DQNPGTAPKY GIRGIPTLLL FKNGEVAATK VGALSKGQLK
EFLDANLAGS GSGLEVLFQG PSHLVKCAEK EKTFCVNGGE CFMVKDLSNP
SRYLCKCPNE FTGDRCQNYV MX^{mFSY}SFYKHLGI EGSGSGSDYK DDDDKAAALE
SEQ ID NO: 175 - TRX
SDKIIHLTDDSFDTDVLKADGAILVDFWAEWCGPCKMIAPILDEIADEYQGKLTVAKLNI
DQNPGTAPKYGIRGIPTLLLFKNGEVAATKVGALSKGQLKEFLDANLA
SEQ ID NO: 176 - NRG1b EGF-Like Domain
SHLVKCAEKE KTFCVNGGEC FMVKDLSNPS RYLCKCPNEF TGDRCQNYVM
X^{mFSY}SFYKHLGIE
SEQ ID NO: 177 - NK035
MA QVQLQESGGG LVQPGGSLRL SCAASGKMSS RRCMAWFRQA PGKERERVAK
LLTTSGSTYL ADSVKGRFTI SQNNAKSTVY LQMNSLKPED TAMYYCAADS
FEDPTCTLVT SSGAFQYWGQ GTQVTVSS GGGGSGGGGSLPETGG

In embodiments of SEQ ID NO:177, one amino acid selected from the group consisting of E102, D103, P104, T105, T107, L108, V109, T110, S111, S112, and G113 is replaced by meta-FSY. In embodiments of SEQ ID NO:177, one amino acid selected from the group consisting of E102, D103, P104, T105, T107, L108, V109, T110, S111, S112, and G113 is replaced by FFY.

NK035
SEQ ID NO: 178
QVQLQESGGG LVQPGGSLRL SCAASGKMSS RRCMAWFRQA
PGKERERVAK LLTTSGSTYL ADSVKGRFTI SQNNAKSTVY
LQMNSLKPED TAMYYCAADS FEDPTCTLVT SSGAFQYWGQ
GTQVTVSS

In embodiments of SEQ ID NO:178, one amino acid selected from the group consisting of E102, D103, P104, T105, T107, L108, V109, T110, 5111, S112, and G113 is replaced by meta-FSY. In embodiments of SEQ ID NO: 178, one amino acid selected from the group consisting of E102, D103, P104, T105, T107, L108, V109, T110, 5111, 5112, and G113 is replaced by FFY.

SEQ ID NO: 179-
NK035
QVQLQESGGG LVQPGGSLRL SCAASGKMSS RRCMAWFRQA PGKERERVAK
LLTTSGSTYL ADSVKGRFTI SQNNAKSTVY LQMNSLKPED TAMYYCAADS
FEDPTCTX^{mFSY}VT SSGAFQYWGQ GTQVTVSS
SEQ ID NO: 180 ZHER2:342 (D37FSY)
VDNKFNKEMRNAYWEIALLPNLNNQQKRAFIRSLYDX^{FSY}PSQSANLLAEAKKLNDAQA
PK
SEQ ID NO: 181-
Nanobody 7D12 CDR3 = AAGSAWYGTLX^{mFSY}EYDY
SEQ ID NO: 182-
Nanobody 7D12 CDR3 = AAGSAWYGTLYEYDX^{mFSY}
SEQ ID NO: 183-
Nanobody 7D12
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLX^{mFSY}E YDYWGQGTQV TVSS
SEQ ID NO: 184-
Nanobody 7D12
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDX^{mFSY}
WGQGTQVTVSS
SEQ ID NO: 185-
Nanobody H11-D4
QVQLVESGGGLMQAGGSLRLSCAVSGRTFSTAAMGWFRQAPGKEREFVAAIRWSGGS
AYYADSVKGRFTISRDKAKNTVYLQMNSLKYEDTAVYYCARTENVRSLLSDYATWPY
X^{FSY}YWGQGTQVTVSSK
SEQ ID NO: 186-
Nanobody H11-D4
QVQLVESGGGLMQAGGSLRLSCAVSGRTFSTAAMGWFRQAPGKEREFVAAIRWSGGS
AYYADSVKGRFTISRDKAKNTVYLQMNSLKYEDTAVYYCARTENVRSLLSDYATWPY
DX^{FSY}WGQG TQVTVSSK
SEQ ID NO: 187-
MR17-K99Y
QVQLVESGGGLVQAGGSLRLSCAASGFPVEVWRMEWYRQAPGKEREGVAAIESYGHG
TRYADSVKGRFTISRDNAKNTVYLQMNSLKPEDTAVYYCNVYDX^{FSY}GQLAYHYDYW
GQGTQVTVSAGRAGEQKLISEEDLNSAVD
SEQ ID NO: 188-
SR4
QVQLVESGGGLVQAGGSLRLSCAASGFPVYSWNMWWYRQAPGKEREWVAAIESX^{FSY}
GDSTRYADSVKGRFTISRDNAKNTVYLQMNSLKPEDTAVYYCYVWVGHTYYGQGTQ
VTVSAGRAGEQKLISEEDLNSAVD
SEQ ID NO: 189-
SR4
QVQLVESGGGLVQAGGSLRLSCAASGFPVYSWNMWWYRQAPGKEREWVAAIESHGD
X^{FSY}TRYADSVKGRFTISRDNAKNTVYLQMNSLKPEDTAVYYCYVWVGHTYYGQGQV
TVSAGRAGEQKLISEEDLNSAVD
SEQ ID NO: 190 = glycine-serine peptide linker-
GGSGGGGGGS
SEQ ID NO: 191 = glycine-serine peptide linker-
GGGSG
SEQ ID NO: 192-
ZHER2: 342 (D36FSY)
VDNKFNKEMR NAYWEIALLP NLNNQQKRAF IRSLYX^{FSY}DPSQ SANLLAEAKK
LNDAQAPK
SEQ ID NO: 193-
ZHER2: 342 (D36mFSY)
VDNKFNKEMR NAYWEIALLP NLNNQQKRAF IRSLYX^{mFSY}DPSQ SANLLAEAKK
LNDAQAPK
SEQ ID NO: 194-
ZHER2: 342 (D37mFSY)
VDNKFNKEMR NAYWEIALLP NLNNQQKRAF IRSLYDX^{mFSY}PSQ SANLLAEAKK
LNDAQAPK
SEQ ID NO: 195-
ZHER2: 2891 (D36FSY)
AEAKYAKEMR NAYWEIALLP NLTNQQKRAF IRKLYX^{FSY}DPSQ
SSELLSEAKKLNDSQAPK
SEQ ID NO: 196-
ZHER2: 2891 (D37FSY)
AEAKYAKEMR NAYWEIALLP NLTNQQKRAF IRKLYDX^{FSY}PSQ
SSELLSEAKKLNDSQAPK
SEQ ID NO: 197-
ZHER2: 2891 (D36mFSY)
AEAKYAKEMR NAYWEIALLP NLTNQQKRAF IRKLYX^{mFSY}DPSQ
SSELLSEAKKLNDSQAPK
SEQ ID NO: 198-
ZHER2: 2891 (D37mFSY)
AEAKYAKEMR NAYWEIALLP NLTNQQKRAF IRKLYDX^{mFSY}PSQ
SSELLSEAKKLNDSQAPK
SEQ ID NO: 199-
Nanobody 7D12 (NbEGFR)
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLYE YDYWGX^{FSY}GTQV TVSS
SEQ ID NO: 200 = DPASPAYGDX^{FSY} = CDR3, mNb6
SEQ ID NO: 201 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYGDX^{FSY}WG QGTQVTVSS
SEQ ID NO: 202 = DPASPAYX^{FSY}DY = CDR3, mNb6
SEQ ID NO: 203 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYX^{FSY}DYWG QGTQVTVSS
SEQ ID NO: 204 = DPASPAYX^{FFY}DY = CDR3, mNb6
SEQ ID NO: 205 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPAYX^{FFY}DYWG QGTQVTVSS
SEQ ID NO: 206 = DPASPX^{FSY}YGDY = CDR3, mNb6
SEQ ID NO: 207 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPX^{FSY}YGDYWG QGTQVTVSS
SEQ ID NO: 208 = DPASPX^{FFY}YGDY = CDR3, mNb6
SEQ ID NO: 209 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
ASPX^{FFY}YGDYWG QGTQVTVSS
SEQ ID NO: 210 = DPAX^{FSY}PAYGDY = CDR3, mNb6
SEQ ID NO: 211 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
AX^{FSY}PAYGDYWG QGTQVTVSS
SEQ ID NO: 212 = DPAX^{FFY}PAYGDY = CDR3, mNb6
SEQ ID NO: 213 = mNb6 nanobody
QVQLVESGGG LVQAGGSLRL SCAASGYIFG RNAMGWYRQA PGKERELVAG
ITRRGSITYY ADSVKGRFTI SRDNAKNTVY LQMNSLKPED TAVYYCAADP
AX^{FFY}PAYGDYWG QGTQVTVSS
SEQ ID NO: 214-
A1
QVQLVQSGGG LVHPGGSLRL SCAASGIDLS LYRMRWYRQA PGKERDLVAL
ITDDGTSYYE DSVKGRFTIT RDNPSNKVFL QMNSLKPEDT AVYYCNAETP
LSPVNYWGQG TQVTVSS
SEQ ID NO: 215-
A1 CDR1 = GIDLSLYR
SEQ ID NO: 216-
A1 CDR2 = ITDDGTS
SEQ ID NO: 217-
A1 CDR3 = NAETPLSPVNY
SEQ ID NO: 218-
A1 CDR1 = X^{FSY}IDLSLYR
SEQ ID NO: 219-
A1 CDR1 = GIX^{FSY}LSLYR
SEQ ID NO: 220-
A1 CDR1 = GIDLX^{FSY}LYR
SEQ ID NO: 221-
A1 CDR1 = GIDLSX^{FSY}YR
SEQ ID NO: 222-
A1 CDR1 = GIDLSLYX^{FSY}
SEQ ID NO: 223-
A1 CDR3 = NAEX^{FSY}PLSPVNY
SEQ ID NO: 224-
A1 CDR3 = NAETX^{FSY}LSPVNY
SEQ ID NO: 225-
A1 CDR3 = NAETPX^{FSY}SPVNY
SEQ ID NO: 226-
A1 CDR3 = NAETPLSX^{FSY}VNY
SEQ ID NO: 227-
A1 G26(FSY)
QVQLVQSGGGLVHPGGSLRLSCAASX^{FSY}IDLSLYRMRWYRQAPGKERDLVALITDDGTSYYEDS
VKGRFT ITRDNPSNKVFLQMNSLKPEDTAVYYCNAETPLSPVNYWGQGTQVTVSS
SEQ ID NO: 228-
A1 D28(FSY)
QVQLVQSGGGLVHPGGSLRLSCAASGIX^{FSY}LSLYRMRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 229-
A1 S30(FSY)
QVQLVQSGGGLVHPGGSLRLSCAASGIDLX^{FSY}ESYLYRMRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 230-
A1 L31(FSY)
QVQLVQSGGGLVHPGGSLRLSCAASGIDLSX^{FSY}YRMRWYRQAPGKERDLVALITDDGTSYYEDS
VKGRFTITRDNPSNKVFLQMNSLKPEDTAVYYCNAETPLSVNYWGQGTQVTVSS
SEQ ID NO: 231-
A1 R33(FSY)
QVQLVQSGGGLVHPGGSLRLSCAASGIDLSLYX^{FSY}MRWYRQAPGKERDLVALITDDGTSYYEDS
VKGRFTITRDNPSNKVFLQMNSLKPEDTAVYYCNAETPLSPVNYWGQGTQVTVSS
SEQ ID NO: 232-
A1 151(FSY)
QVQLVQSGGGLVHPGGSLRLSCAASGIDLSLYRMRWYRQAPGKERDLVALX^{FSY}TDDGTSYYEDS
VKGRFTITRDNPSNKVFLQMNSLKPEDTAVYYCNAETPLSPVNYWGQGTQVTVSS
SEQ ID NO: 233-
A1 D53(FSY)
QVQLVQSGGGLVHPGGSLRLSCAASGIDLSLYRMRWYRQAPGKERDLVALITX^{FSY}DGTSYYEDS
VKGRFTITRDNPSNKVFLQMNSLKPEDTAVYYCNAETPLSPVNYWGQGTQVTVSS
SEQ ID NO: 234-
A1 D54(FSY)
QVQLVQSGGGLVHPGGSLRLSCAASGIDLSLYRMRWYRQAPGKERDLVALITDX^{FSY}GTSYYEDS
VKGRFTITRDNPSNKVFLQMNSLKPEDTAVYYCNAETPLSPVNYWGQGTQVTVSS
SEQ ID NO: 235-
A1 T99(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAEX^{FSY}ESYPLSPVNYWGQGTQVTVSS
SEQ ID NO: 236-
A1 P100(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAETX^{FSY}LSPVNYWGQGTQVTVSS
SEQ ID NO: 237-
A1 L101(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAETPX^{FSY}ESYSPVNYWGQGTQVTVSS
SEQ ID NO: 238-
A1 P103(FSY)
QVQLVQSGGGLVHPGGSLRLSCAASGIDLSLYRMRWYRQAPGKERDLVALITDDGTSYYEDSVK
GRFTITRDNPSNKVFLQMNSLKPEDTAVYYCNAETPLSX^{FSY}VNYWGQGTQVTVSS
SEQ ID NO: 239-
C6
QVQLVQSGGG LVQAGGSLRL SCAPSGSIFG IRTMDWYRQA PGKERELVAR
ITMDGRVFHA DSVKGRESGS RDGASNAVYL QMNSLKPDDT AVYYCRYSGL
TSREDYWGPG TQVTVSS
SEQ ID NO: 240-
C6 CDR1 = GSIFGIRT
SEQ ID NO: 241-
C6 CDR2 = ITMDGRV
SEQ ID NO: 242-
C6 CDR3 = RYSGLTSREDY
SEQ ID NO: 243-
C6 CDR1 = GX^{FSY}IFGIRT
SEQ ID NO: 244-
C6 CDR1 = GSIXESYGIRT
SEQ ID NO: 245-
C6 CDR1 = GSIFGX^{FSY}RT
SEQ ID NO: 246-
C6 CDR1 = GSIFGIX^{FSY}ESYT
SEQ ID NO: 247-
C6 CDR2 = IX^{FSY}MDGRV
SEQ ID NO: 248-
C6 CDR2 = ITX^{FSY}DGRV
SEQ ID NO: 249-
C6 CDR2 = ITMX^{FSY}GRV
SEQ ID NO: 250-
C6 CDR2 = ITMDX^{FSY}RV
SEQ ID NO: 251-
C6 CDR3 = X^{FSY}YSGLTSREDY
SEQ ID NO: 252-
C6 CDR3 = RYSGLX^{FSY}SREDY
SEQ ID NO: 253-
C6 CDR3 = RYSGLTX^{FSY}REDY
SEQ ID NO: 254-
C6 CDR3 = RYSGLTSREX^{FSY}Y
SEQ ID NO: 255-
C6 S27(FSY)
QVQLVQSGGGLVQAGGSLRLSCAPSGX^{FSY}IFGIRTMDWYRQAPGKERELVARITMDGRVFHADS
VKGRFSGSRDGASNAVYLQMNSLKPDDTAVYYCRYSGLTSREDYWGPGTQVTVSS
SEQ ID NO: 256-
C6 F29(FSY)
QVQLVQSGGGLVQAGGSLRLSCAPSGSIX^{FSY}SYGIRTMDWYRQAPGKERELVARITMDGRVFHADS
VKGRFSGSRDGASNAVYLQMNSLKPDDTAVYYCRYSGLTSREDYWGPGTQVTVSS
SEQ ID NO: 257-
C6 131(FSY)
QVQLVQSGGGLVQAGGSLRLSCAPSGSIFGX^{FSY}RTMDWYRQAPGKERELVARITMDGRVFHADS
VKGRFSGSRDGASNAVYLQMNSLKPDDTAVYYCRYSGLTSREDYWGPGTQVTVSS
SEQ ID NO: 258-
C6 R32(FSY)
QVQLVQSGGGLVQAGGSLRLSCAPSGSIFGIX^{FSY}TMDWYRQAPGKERELVARITMDGRVFHADS
VKGRFSGSRDGASNAVYLQMNSLKPDDTAVYYCRYSGLTSREDYWGPGTQVTVSS
SEQ ID NO: 259-
C6 T52(FSY)
QVQLVQSGGGLVQAGGSLRLSCAPSGSIFGIRTMDWYRQAPGKERELVARIX^{FSY}MDGRVFHADS
VKGRFSGSRDGASNAVYLQMNSLKPDDTAVYYCRYSGLTSREDYWGPGTQVTVSS
SEQ ID NO: 260-
C6 M53(FSY)
QVQLVQSGGGLVQAGGSLRLSCAPSGSIFGIRTMDWYRQAPGKERELVARITX^{FSY}SYDGRVFHADS
VKGRFSGSRDGASNAVYLQMNSLKPDDTAVYYCRYSGLTSREDYWGPGTQVTVSS
SEQ ID NO: 261-
C6 D54(FSY)
QVQLVQSGGGLVQAGGSLRLSCAPSGSIFGIRTMDWYRQAPGKERELVARITMX^{FSY}GRVFHADS
VKGRFSGSRDGASNAVYLQMNSLKPDDTAVYYCRYSGLTSREDYWGPGTQVTVSS
SEQ ID NO: 262-
C6 G55(FSY)
QVQLVQSGGGLVQAGGSLRLSCAPSGSIFGIRTMDWYRQAPGKERELVARITMDX^{FSY}RVFHADS
VKGRFSGSRDGASNAVYLQMNSLKPDDTAVYYCRYSGLTSREDYWGPGTQVTVSS
SEQ ID NO: 263-
C6 R96(FSY)
QVQLVQSGGGLVQAGGSLRLSCAPSGSIFGIRTMDWYRQAPGKERELVARITMDGRVFHADSVK
GRESGSRDGASNAVYLQMNSLKPDDTAVYYCX^{FSY}SYYSGLTSREDYWGPGTQVTVSS
SEQ ID NO: 264-
C6 T101(FSY)
QVQLVQSGGGLVQAGGSLRLSCAPSGSIFGIRTMDWYRQAPGKERELVARITMDGRVFHADSVK
GRESGSRDGASNAVYLQMNSLKPDDTAVYYCRYSGLX^{FSY}SREDYWGPGTQVTVSS
SEQ ID NO: 265-
C6 S102(FSY)
QVQLVQSGGGLVQAGGSLRLSCAPSGSIFGIRTMDWYRQAPGKERELVARITMDGRVFHADSVK
GRESGSRDGASNAVYLQMNSLKPDDTAVYYCRYSGLTX^{FSY}SYREDYWGPGTQVTVSS
SEQ ID NO: 266-
C6 D105(FSY)
QVQLVQSGGGLVQAGGSLRLSCAPSGSIFGIRTMDWYRQAPGKERELVARITMDGRVFHADSVK
GRESGSRDGASNAVYLQMNSLKPDDTAVYYCRYSGLTSREX^{FSY}SYYWGPGTQVTVSS
SEQ ID NO: 267 = SR4 S57(FSY) nanobody amino acid seuqnence:
QVQLVESGGG LVQAGGSLRL SCAASGFPVY SWNMWWYRQA PGKEREWVAA
IESHGDX^{FSY}SYTRY ADSVKGRFTI SRDNAKNTVY LQMNSLKPED TAVYYCYVWV
GHTYYGQGTQVTVSAGRAGEQKLISEEDLNSAVD
SEQ ID NO: 268 SR4 nanobody-
CDR2 = AIESHGDX^{FSY}STR

### Embodiments S1-S59

Embodiment S1. A single-domain antibody comprising an unnatural amino acid side chain; wherein the single-domain antibody comprises: (1) a region comprising CDR1 as set forth in SEQ ID NO:31, a region comprising CDR2 as set forth in SEQ ID NO:32; and a region comprising CDR3 as set forth in SEQ ID NO:33; (2) a region comprising CDR1 as set forth in SEQ ID NO:35, a region comprising CDR2 as set forth in SEQ ID NO:36; and a region comprising CDR3 as set forth in SEQ ID NO:37; or (3) a region comprising CDR1 as set forth in SEQ ID NO:39, a region comprising CDR2 as set forth in SEQ ID NO:40; and a region comprising CDR3 as set forth in SEQ ID NO:41

Embodiment S2. The single-domain antibody of Embodiment S1, wherein the unnatural amino acid side chain is a moiety of the formula:

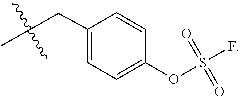 [SMILES: *Cc1ccc(OS(=O)(=O)F)cc1]

Embodiment S3. The single-domain antibody of Embodiment S1 or S2, comprising (1) the region comprising CDR1 as set forth in SEQ ID NO:31, the region comprising CDR2 as set forth in SEQ ID NO:32; and the region comprising CDR3 as set forth in SEQ ID NO:33.

Embodiment S4. The single-domain antibody of Embodiment S3, wherein the unnatural amino acid side chain is in the region comprising CDR2 as set forth in SEQ ID NO:32.

Embodiment S5. The single-domain antibody of Embodiment S4, wherein the unnatural amino acid side chain is at a position corresponding to position 5 in SEQ ID NO:32.

Embodiment S6. The single-domain antibody of Embodiment S4, wherein the unnatural amino acid side chain is at a position corresponding to position 8 in SEQ ID NO:32.

Embodiment S7. The single-domain antibody of any one of Embodiments S3 to S6, wherein the single domain antibody has at least 85% sequence identity to SEQ ID NO:30.

Embodiment S8. The single-domain antibody of Embodiment S1 or S2, comprising (2) the region comprising CDR1 as set forth in SEQ ID NO:35, the region comprising CDR2 as set forth in SEQ ID NO:36; and the region comprising CDR3 as set forth in SEQ ID NO:37.

Embodiment S9. The single-domain antibody of Embodiment S8, wherein the unnatural amino acid side chain is in the region comprising CDR3 as set forth in SEQ ID NO:37.

Embodiment S10. The single-domain antibody of Embodiment S9, wherein the unnatural amino acid side chain is at a position corresponding to position 4 in SEQ ID NO:37.

Embodiment S11. The single-domain antibody of any one of Embodiments S8 to S10, wherein the single domain antibody has at least 85% sequence identity to SEQ ID NO:34.

Embodiment S12. The single-domain antibody of Embodiment S1 or S2, comprising the single domain antibody has at least 85% sequence identity to SEQ ID NO:34.

Embodiment S13. The single-domain antibody of Embodiment S12, wherein the unnatural amino acid side chain is in the region comprising CDR3 as set forth in SEQ ID NO:41.

Embodiment S14. The single-domain antibody of Embodiment S13, wherein the unnatural amino acid side chain is at a position corresponding to position 18 in SEQ ID NO:41.

Embodiment S15. The single-domain antibody of Embodiment S13, wherein the unnatural amino acid side chain is at a position corresponding to position 19 in SEQ ID NO:41.

Embodiment S16. The single-domain antibody of any one of Embodiments S12 to S15, wherein the single domain antibody has at least 85% sequence identity to SEQ ID NO:38.

Embodiment S17. A single-domain antibody comprising an unnatural amino acid side chain; wherein the unnatural amino acid side chain is a moiety of the formula:

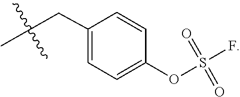 [SMILES: *Cc1ccc(OS(=O)(=O)F)cc1]

Embodiment S18. The single-domain antibody of any one of Embodiments S1 to S17, wherein the unnatural amino acid side chain is capable of covalently binding to a lysine, tyrosine, or histidine on a viral spike (S) protein of a SARS-coronavirus.

Embodiment S19. The single-domain antibody of Embodiment S18, wherein the SARS-coronavirus is SARS-CoV-2.

Embodiment S20. A pharmaceutical composition comprising the single-domain antibody of any one of Embodiments Si to S19.

Embodiment S21. A nucleic acid encoding the single-domain antibody of any one of Embodiments S1 to S19.

Embodiment S22. A nucleic acid having at least 80% sequence identity to SEQ ID NO:42, SEQ ID NO:43, or SEQ ID NO:44.

Embodiment S23. A plasmid comprising the nucleic acid of Embodiment S21 or S22.

Embodiment S24. A method of treating or preventing COVID-19 in a subject in need thereof, the method comprising administering to the subject an effective amount of the single-domain antibody of any one of Embodiments S1 to S19, the pharmaceutical composition of Embodiment S20, the nucleic acid of Embodiment S21 or S22, or the plasmid of Embodiment S23.

Embodiment S25. A method of treating or preventing a SARS-coronavirus infection in a subject in need thereof, the method comprising administering to the subject an effective amount of the single-domain antibody of any one of Embodiments S1 to S19, the pharmaceutical composition of Embodiment S20, the nucleic acid of Embodiment S21 or 2S2, or the plasmid of Embodiment S23.

Embodiment S26. The method of Embodiment S25, wherein the SARS-coronavirus infection is a SARS-CoV-2 infection.

Embodiment S27. A protein complex comprising the single-domain antibody of any one of Embodiments S1 to S19 covalently linked via the unnatural amino acid side chain of Formula (II) to a lysine, a tyrosine, or a histidine on a viral spike (S) protein of a SARS-coronavirus.

Embodiment S28. A protein complex comprising a single-domain antibody linked via an unnatural amino acid side chain of Formula (II) to a lysine, a tyrosine, or a histidine on a viral spike (S) protein of a SARS-coronavirus; wherein the unnatural amino acid side chain is a moiety of the formula:

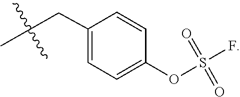 [SMILES: *Cc1ccc(OS(=O)(=O)F)cc1]

Embodiment S29. The protein complex of Embodiment S27 or S28, wherein the SARS-coronavirus is SARS-CoV-2.

Embodiment S30. The protein complex of any one of Embodiments S27 or S29, wherein the viral spike (S) protein has at least 80% sequence identity to SEQ ID NO:5.

Embodiment 3S1. The protein complex of Embodiment S30, wherein the viral spike (S) protein comprises one or more mutations selected from the group consisting of K417N, N439K, E484K, F490L, and N501Y.

Embodiment S32. A SARS-coronavirus comprising the protein complex of one of Embodiments S27 to S31.

Embodiment S33. A recombinant protein comprising an ACE2 receptor protein having an unnatural amino acid side chain; wherein the unnatural amino acid side chain is capable of covalently binding to a lysine, tyrosine, or histidine.

Embodiment S34. The recombinant protein of Embodiment S33, wherein the unnatural amino acid side chain is a moiety of formula:

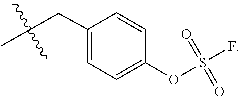 [SMILES: *Cc1ccc(OS(=O)(=O)F)cc1]

Embodiment S35. The recombinant protein of Embodiment S33 or S34, wherein the unnatural amino acid side chain is at a position corresponding to position 34, 37, or 42 in the ACE2 receptor protein.

Embodiment S36. The recombinant protein of any one of Embodiments S33 to S35, wherein the ACE receptor protein has at least 80% sequence identity to SEQ ID NO:1.

Embodiment S37. The recombinant protein of any one of Embodiments S33 to S36, wherein the ACE receptor protein has at least 80% sequence identity to the region spanning amino acid residue 19 to amino acid residue 615 in SEQ ID NO:1.

Embodiment S38. The recombinant protein of any one of Embodiments S33 to S37, further comprising an Fc fragment.

Embodiment S39. The recombinant protein of Embodiment S38, wherein the Fc fragment is an IgG Fc fragment.

Embodiment S40. The recombinant protein of any one of Embodiments S33 to S39, further comprising an epitope tag.

Embodiment S41. The recombinant protein of any one of Embodiments S33 to S40, wherein the unnatural amino acid side chain is capable of covalently binding to a lysine, tyrosine, or histidine on a viral spike (S) protein of a SARS-coronavirus.

Embodiment S42. The recombinant protein of Embodiment S41, wherein the unnatural amino acid side chain is capable of covalently binding to a tyrosine on a viral spike (S) protein of a SARS-coronavirus.

Embodiment S43. The recombinant protein of Embodiment S41 or S42, wherein the SARS-coronavirus is SARS-CoV-2.

Embodiment S44. The recombinant protein of Embodiment S43, wherein the viral spike (S) protein of the SARS-CoV-2 has at least 80% sequence identity to SEQ ID NO:5.

Embodiment S45. The recombinant protein of Embodiment S44, wherein the viral spike (S) protein comprises one or more mutations selected from the group consisting of K417N, N439K, E484K, F490L, and N501Y.

Embodiment S46. A pharmaceutical composition comprising the recombinant protein of any one of Embodiments S33 to S45.

Embodiment S47. A nucleic acid encoding the recombinant protein of any one of Embodiments S33 to S45.

Embodiment S48. A nucleic acid having at least 80% sequence identity to SEQ ID NO:21, SEQ ID NO:23, SEQ ID NO:24, SEQ ID NO:25, SEQ ID NO:27, or SEQ ID NO:28.

Embodiment S49. A plasmid comprising the nucleic acid of Embodiment S47 or S48.

Embodiment S50. A method of treating or preventing COVID-19 in a subject in need thereof, the method comprising administering to the subject an effective amount of the recombinant protein of any one of Embodiments S33 to S45, the pharmaceutical composition of Embodiment S46, the nucleic acid of Embodiment S47 or S48, or the plasmid of Embodiment S49.

Embodiment S51. A method of treating or preventing a SARS-coronavirus infection in a subject in need thereof, the method comprising administering to the subject an effective amount of the recombinant protein of any one of Embodiments S33 to S45, the pharmaceutical composition of Embodiment S46, the nucleic acid of Embodiment S47 or S48, or the plasmid of Embodiment S49.

Embodiment S52. The method of Embodiment S51, wherein the SARS-coronavirus infection is a SARS-CoV-2 infection.

Embodiment 553. A protein complex comprising the recombinant protein of any one of Embodiments S33 to S45 covalently linked to lysine, tyrosine, or histidine on a viral spike (S) protein of a SARS-coronavirus.

Embodiment 554. The protein complex of Embodiment S53, wherein the SARS-coronavirus is SARS-coronavirus 2.

Embodiment S55. The protein complex of Embodiment S53 or S54, wherein the viral spike (S) protein has at least 80% sequence identity to SEQ ID NO:5.

Embodiment S56. The protein complex of Embodiment S55, wherein the viral spike (S) protein comprises one or more mutations selected from the group consisting of K417N, N439K, E484K, F490L, and N501Y.

Embodiment S57. The protein complex of any one of Embodiments S53 to S56, wherein the recombinant protein is covalently linked to a tyrosine on the viral spike (S) protein.

Embodiment S58. The protein complex of Embodiment S57, wherein the recombinant protein is covalently linked to the viral spike (S) protein at a position corresponding to position Y453, Y505, or Y449 in the SARS-coronavirus.

Embodiment S59. A SARS-coronavirus comprising the protein complex of one of Embodiments S53 to S58.

### Embodiments 1-242

Embodiment 1. A RNA-binding protein comprising an unnatural amino acid, wherein the unnatural amino acid comprises a side chain of Formula (II):

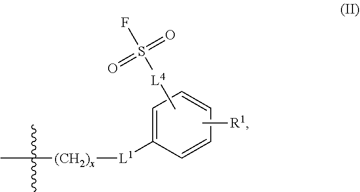

wherein: the RNA binding protein is a CRISPR protein or a RNA chaperone; L⁴ is a bond or —O—; x is an integer from 1 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment 2. The RNA-binding protein of Embodiment 1, wherein L⁴ is a bond.

Embodiment 3. The RNA-binding protein of Embodiment 1, wherein L⁴ is —O—.

Embodiment 4. The RNA-binding protein of any one of Embodiments 1 to 3, wherein x is an integer from 1 to 4.

Embodiment 5. The RNA-binding protein of any one of Embodiment 4, wherein x is 1.

Embodiment 6. The RNA-binding protein of any one of Embodiments 1 to 5, wherein L¹ is a bond.

Embodiment 7. The RNA-binding protein of any one of Embodiments 1 to 5, wherein L¹ is substituted or unsubstituted 2 to 6 membered heteroalkylene.

Embodiment 8. The RNA-binding protein of Embodiment 7, wherein L¹ is —NH—C(O)—(CH₂)_{y}— or —NH—C(O)—O—(CH₂)_{y}—, and y is an integer from 0 to 2.

Embodiment 9. The RNA-binding protein of any one of Embodiments 1 to 8, wherein R′ is substituted or unsubstituted heteroalkyl.

Embodiment 10. The RNA-binding protein of Embodiment 9, wherein R¹ is unsubstituted 2 to 8 membered heteroalkyl.

Embodiment 11. The RNA-binding protein of Embodiment 10, wherein R¹ is —O—(CH₂)ₘCH₃, and m is an integer from 0 to 4.

Embodiment 12. The RNA-binding protein of any one of Embodiments 1 to 11, wherein R′ is ortho to —S(═O)₂F.

Embodiment 13. The RNA-binding protein of any one of Embodiments 1 to 8, wherein R¹ is hydrogen.

Embodiment 14. The RNA-binding protein of Embodiment 1, wherein the side chain of Formula (II) has the structure of Formula (IIC):

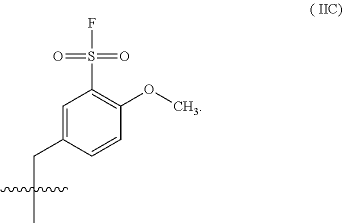 [SMILES: *Cc1ccc(OC)c(S(=O)(=O)F)c1]

Embodiment 15. The RNA-binding protein of Embodiment 1, wherein the side chain of Formula (II) has the structure of Formula (TIE):

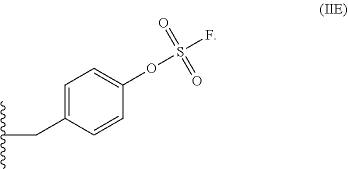 [SMILES: *Cc1ccc(OS(=O)(=O)F)cc1]

Embodiment 16. The RNA binding protein of any one of Embodiments 1 to 15, wherein the RNA binding protein is the CRISPR protein.

Embodiment 17. The RNA binding protein of Embodiment 16, wherein the CRISPR protein comprises the unnatural amino acid sidechain at a position corresponding to position 133 or position 380, with reference to the amino acid sequence of catalytically inactive Cas13b protein from Prevotella sp. P5-125.

Embodiment 18. The RNA binding protein of Embodiment 16 or 17, wherein the CRISPR protein comprises the unnatural amino acid sidechain at a position corresponding to position 128, position 133, position 380, position 1053, or position 1058, with reference to the amino acid sequence of catalytically inactive Cas13b protein from Prevotella sp. P5-125.

Embodiment 19. The RNA binding protein of Embodiment 16, wherein the CRISPR protein is a catalytically inactive Cas13b protein.

Embodiment 20. The RNA binding protein of Embodiment 19, wherein the catalytically inactive Cas13b protein is from Prevotella sp. P5-125, Bergeyella zoohelcum, or Prevotella buccae.

Embodiment 21. The RNA binding protein of Embodiment 19 or 20, wherein the catalytically inactive Cas13b protein comprises the unnatural amino acid sidechain at a position corresponding to position 133 or position 380.

Embodiment 22. The RNA binding protein of Embodiment 20, wherein the catalytically inactive Cas13b protein from Prevotella sp. P5-125 comprises the unnatural amino acid sidechain at a position corresponding to position R128, H133, R380, R1053, H1058, or two or more thereof; the catalytically inactive Cas13b protein from Bergeyella zoohelcum comprises the unnatural amino acid sidechain at a position corresponding to position R116, H121, R459, R1177, H1182, or two or more thereof, and the catalytically inactive Cas13b protein from Prevotella buccae comprises the unnatural amino acid sidechain at a position corresponding to position R156, H161, K393, R402, R1068, H1073, or two or more thereof.

Embodiment 23. The RNA binding protein of Embodiment 16, wherein the CRISPR protein is a catalytically inactive Cas9 protein.

Embodiment 24. The RNA binding protein of Embodiment 23, wherein the catalytically inactive Cas9 protein is from Streptococcus pyogenes, Staphylococcus aureus, or Actinomyces naeslundii.

Embodiment 25. The RNA binding protein of Embodiment 24, wherein the catalytically inactive Cas9 protein from Streptococcus pyogenes comprises the unnatural amino acid sidechain at a position corresponding to position D10, E762, H983, D986, H840, N863, D839, or two or more thereof, the catalytically inactive Cas9 protein from Staphylococcus aureus comprises the unnatural amino acid sidechain at a position corresponding to position D10, E477, H701, D704, H557, N580, D556, or two or more thereof, and the catalytically inactive Cas9 protein from Actinomyces naeslundii comprises the unnatural amino acid sidechain at a position corresponding to position D17, E505, H736, D739, H582, N606, D581, or two or more thereof.

Embodiment 26. The RNA binding protein of Embodiment 16, wherein the CRISPR protein is a catalytically inactive Cas12a protein.

Embodiment 27. The RNA binding protein of Embodiment 26, wherein the catalytically inactive Cas12a protein is from Acidaminococcus sp. BV3L6, Lachnospiraceae bacterium ND2006, or Francisella novicida U112.

Embodiment 28. The RNA binding protein of Embodiment 27, wherein the catalytically inactive Cas12a protein from Acidaminococcus sp. BV3L6 comprises the unnatural amino acid sidechain at a position corresponding to position D908, E993, D1263, R1226, D1235, or two or more thereof; the catalytically inactive Cas12a protein from Lachnospiraceae bacterium ND2006 comprises the unnatural amino acid sidechain at a position corresponding to position D833, E926, D1181, R1139, D1149, or two or more thereof; and the catalytically inactive Cas12a protein from Francisella novicida U112 comprises the unnatural amino acid sidechain at a position corresponding to position D917, E1006, D1255, R1218, D1226, or two or more thereof.

Embodiment 29. The RNA binding protein of Embodiment 16, wherein the CRISPR protein is a catalytically inactive Cas13a protein.

Embodiment 30. The RNA binding protein of Embodiment 29, wherein the catalytically inactive Cas13a protein is from Leptotrichia buccalis or Leptotrichia wadei.

Embodiment 31. The RNA binding protein of Embodiment 30, wherein the catalytically inactive Cas13a protein from Leptotrichia buccalis comprises the unnatural amino acid sidechain at a position corresponding to position K47, R472, H473, H477, S522, D590, Q659, V810, K855, Q904, R1046, H1053, R1135, or two or more thereof; and the catalytically inactive Cas13a protein from Leptotrichia wadei comprises the unnatural amino acid sidechain at a position corresponding to position K47, R474, H475, H479, S524, D586, Q653, V808, K853, Q902, R1046, H1051, R1133, or two or more thereof.

Embodiment 32. The RNA binding protein of Embodiment 16, wherein the CRISPR protein is a catalytically inactive Cas13d protein.

Embodiment 33. The RNA binding protein of Embodiment 32, wherein the catalytically inactive Cas13d protein is from Eubacterium siraeum.

Embodiment 34. The RNA binding protein of Embodiment 33, wherein the catalytically inactive Cas13d protein from Eubacterium siraeum comprises the unnatural amino acid sidechain at a position corresponding to position R84, N86, R386, N405, T524, N641, R679, Y680, or two or more thereof.

Embodiment 35. The RNA binding protein of any one of Embodiments 1 to 15, wherein the RNA binding protein is the RNA chaperone.

Embodiment 36. The RNA binding protein of Embodiment 35, wherein the RNA chaperone is a Hfq protein.

Embodiment 37. The RNA binding protein of Embodiment 36, wherein the Hfq protein comprises the unnatural amino acid sidechain at a position corresponding to position 25, position 30, or position 49.

Embodiment 38. A nucleic acid encoding the CRISPR protein of any one of Embodiments 1 to 37.

Embodiment 39. A vector comprising the nucleic acid sequence of Embodiment 38.

Embodiment 40. A biomolecule conjugate of Formula (III):

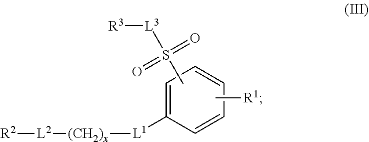

wherein: R² is a CRISPR protein moiety or a RNA chaperone moiety; R³ is a RNA moiety; L¹ is a bond, substituted or unsubstituted alkylene, or substituted or unsubstituted heteroalkylene; x is an integer from 1 to 8; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; v1 is 1 or 2; L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 41. The biomolecule conjugate of Embodiment 40, wherein L⁴ is a bond.

Embodiment 42. The biomolecule conjugate of Embodiment 40, wherein L⁴ is —O—.

Embodiment 43. The biomolecule conjugate of any one of Embodiments 40 to 42, wherein x is an integer from 1 to 4.

Embodiment 44. The biomolecule conjugate of any one of Embodiment 40, wherein x is 1.

Embodiment 45. The biomolecule conjugate of any one of Embodiments 40 to 44, wherein L¹ is a bond.

Embodiment 46. The biomolecule conjugate of any one of Embodiments 40 to 44, wherein L¹ is substituted or unsubstituted 2 to 6 membered heteroalkylene.

Embodiment 47. The biomolecule conjugate of Embodiment 46, wherein L¹ is —NH—C(O)—(CH₂)_{y}— or —NH—C(O)—O—(CH₂)_{y}—, and y is an integer from 0 to 2.

Embodiment 48. The biomolecule conjugate of any one of Embodiments 40 to 47, wherein R¹ is substituted or unsubstituted heteroalkyl.

Embodiment 49. The biomolecule conjugate of Embodiment 48, wherein R¹ is unsubstituted 2 to 8 membered heteroalkyl.

Embodiment 50. The biomolecule conjugate of Embodiment 49, wherein R¹ is —O—(CH₂)ₘCH₃, and m is an integer from 0 to 4.

Embodiment 51. The biomolecule conjugate of any one of Embodiments 40 to 50, wherein R¹ is ortho to —S(═O)₂F.

Embodiment 52. The biomolecule conjugate of any one of Embodiments 40 to 47, wherein R¹ is hydrogen.

Embodiment 53. The biomolecule conjugate of any one of Embodiments 40 to 52, wherein: L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-substituted or unsubstituted heteroarylene; L¹² is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl; L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹²-substituted or unsubstituted heteroarylene; and L¹³ is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl.

Embodiment 54. The biomolecule conjugate of Embodiment 40, wherein the biomolecule conjugate of Formula (III) is a biomolecule conjugate of Formula (IIIC):

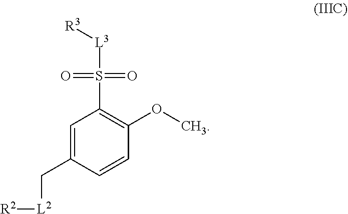

Embodiment 55. The biomolecule conjugate of Embodiment 40, wherein the biomolecule conjugate of Formula (III) is a biomolecule conjugate of Formula (IIIE):

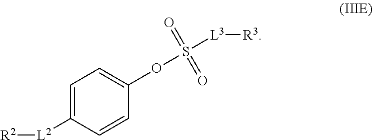

Embodiment 56. The biomolecule conjugate of any one of Embodiments 40 to 55, wherein L² is a bond.

Embodiment 57. The biomolecule conjugate of any one of Embodiments 40 to 55, wherein L³ is a bond.

Embodiment 58. The biomolecule conjugate of any one of Embodiments 40 to 57, wherein the RNA binding protein is the CRISPR protein.

Embodiment 59. The biomolecule conjugate of Embodiment 58, wherein the CRISPR protein comprises the unnatural amino acid sidechain at a position corresponding to position 133.

Embodiment 60. The biomolecule conjugate of Embodiment 58 or 59, wherein the CRISPR protein comprises the unnatural amino acid sidechain at a position corresponding to position 380.

Embodiment 61. The biomolecule conjugate of Embodiment 58, wherein the CRISPR protein is a catalytically inactive Cas13b protein.

Embodiment 62. The biomolecule conjugate of Embodiment 61, wherein the catalytically inactive Cas13b protein is from Prevotella sp. P5-125, Bergeyella zoohelcum, or Prevotella buccae.

Embodiment 63. The biomolecule conjugate of Embodiment 61 or 62, wherein the catalytically inactive Cas13b protein comprises the unnatural amino acid sidechain at a position corresponding to position 133 or position 380.

Embodiment 64. The biomolecule conjugate of Embodiment 62, wherein the catalytically inactive Cas13b protein from Prevotella sp. P5-125 comprises the unnatural amino acid sidechain at a position corresponding to position R128, H133, R380, R1053, H1058, or two or more thereof; the catalytically inactive Cas13b protein from Bergeyella zoohelcum comprises the unnatural amino acid sidechain at a position corresponding to position R116, H121, R459, R1177, H1182, or two or more thereof, and the catalytically inactive Cas13b protein from Prevotella buccae comprises the unnatural amino acid sidechain at a position corresponding to position R156, H161, K393, R402, R1068, H1073, or two or more thereof.

Embodiment 65. The biomolecule conjugate of Embodiment 58, wherein the CRISPR protein is a catalytically inactive Cas9 protein.

Embodiment 66. The biomolecule conjugate of Embodiment 65, wherein the catalytically inactive Cas9 protein is from Streptococcus pyogenes, Staphylococcus aureus, or Actinomyces naeslundii.

Embodiment 67. The biomolecule conjugate of Embodiment 66, wherein the catalytically inactive Cas9 protein from Streptococcus pyogenes comprises the unnatural amino acid sidechain at a position corresponding to position D10, E762, H983, D986, H840, N863, D839, or two or more thereof, the catalytically inactive Cas9 protein from Staphylococcus aureus comprises the unnatural amino acid sidechain at a position corresponding to position D10, E477, H701, D704, H557, N580, D556, or two or more thereof; and the catalytically inactive Cas9 protein from Actinomyces naeslundii comprises the unnatural amino acid sidechain at a position corresponding to position D17, E505, H736, D739, H582, N606, D581, or two or more thereof.

Embodiment 68. The biomolecule conjugate of Embodiment 58, wherein the CRISPR protein is a catalytically inactive Cas12a protein.

Embodiment 69. The biomolecule conjugate of Embodiment 68, wherein the catalytically inactive Cas12a protein is from Acidaminococcus sp. BV3L6, Lachnospiraceae bacterium ND2006, or Francisella novicida U112.

Embodiment 70. The biomolecule conjugate of Embodiment 69, wherein the catalytically inactive Cas12a protein from Acidaminococcus sp. BV3L6 comprises the unnatural amino acid sidechain at a position corresponding to position D908, E993, D1263, R1226, D1235, or two or more thereof; the catalytically inactive Cas12a protein from Lachnospiraceae bacterium ND2006 comprises the unnatural amino acid sidechain at a position corresponding to position D833, E926, D1181, R1139, D1149, or two or more thereof; and the catalytically inactive Cas12a protein from Francisella novicida U112 comprises the unnatural amino acid sidechain at a position corresponding to position D917, E1006, D1255, R1218, D1226, or two or more thereof.

Embodiment 71. The biomolecule conjugate of Embodiment 58, wherein the CRISPR protein is a catalytically inactive Cas13a protein.

Embodiment 72. The biomolecule conjugate of Embodiment 71, wherein the catalytically inactive Cas13a protein is from Leptotrichia buccalis or Leptotrichia wadei.

Embodiment 73. The biomolecule conjugate of Embodiment 72, wherein the catalytically inactive Cas13a protein from Leptotrichia buccalis comprises the unnatural amino acid sidechain at a position corresponding to position K47, R472, H473, H477, S522, D590, Q659, V810, K855, Q904, R1046, H1053, R1135, or two or more thereof; and the catalytically inactive Cas13a protein from Leptotrichia wadei comprises the unnatural amino acid sidechain at a position corresponding to position K47, R474, H475, H479, S524, D586, Q653, V808, K853, Q902, R1046, H1051, R1133, or two or more thereof.

Embodiment 74. The biomolecule conjugate of Embodiment 58, wherein the CRISPR protein is a catalytically inactive Cas13d protein.

Embodiment 75. The biomolecule conjugate of Embodiment 74, wherein the catalytically inactive Cas13d protein is from Eubacterium siraeum.

Embodiment 76. The biomolecule conjugate of Embodiment 75, wherein the catalytically inactive Cas13d protein from Eubacterium siraeum comprises the unnatural amino acid sidechain at a position corresponding to position R84, N₈₆, R386, N405, T524, N641, R679, Y680, or two or more thereof.

Embodiment 77. The biomolecule conjugate of any one of Embodiments 40 to 57, wherein the RNA binding protein is the RNA chaperone.

Embodiment 78. The biomolecule conjugate of Embodiment 77, wherein the RNA chaperone is a Hfq protein.

Embodiment 79. The biomolecule conjugate of Embodiment 78, wherein L² is bonded to the Hfq protein at a position corresponding to position 25, position 30, or position 49.

Embodiment 80. A method of forming the biomolecule conjugate of any one of Embodiments 40 to 79, the method comprising contacting the RNA-binding protein of any one of Embodiments 1 to 39, RNA, and a guide RNA (crRNA), thereby forming the biomolecule conjugate.

Embodiment 81. A cell comprising: (i) the RNA-binding protein of any one of Embodiments 1 to 37; (ii) the nucleic acid of Embodiment 38; (iii) the vector of Embodiment 39; pr (iv) the biomolecule conjugate of any one of Embodiments 40 to 79.

Embodiment 82. The cell of Embodiment 81, wherein the cell is a bacterial cell or a mammalian cell.

Embodiment 83. A compound of Formula (IV):

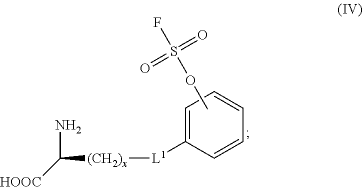

wherein: —OS(═O)₂F is meta or ortho to the carbon atom linked to L¹; x is an integer from 1 to 8; and L¹ is a bond, substituted or unsubstituted alkylene, or substituted or unsubstituted heteroalkylene.

Embodiment 84. The compound of Embodiment 83, wherein x is an integer from ito 4.

Embodiment 85. The compound of Embodiment 83 or 84, wherein L¹ is a bond.

Embodiment 86. The compound of Embodiment 83 or 84, wherein L¹ is substituted or unsubstituted 2 to 6 membered heteroalkylene.

Embodiment 87. The compound of Embodiment 83 or 84, wherein L¹ is —NH—C(O)—(CH₂)_{y}— or —NH—C(O)—O—(CH₂)_{y}—, and y is an integer from 0 to 2.

Embodiment 88. The compound of any one of Embodiments 83 to 87, wherein —OS(═O)₂F is ortho to the carbon atom linked to L¹.

Embodiment 89. The compound of any one of Embodiments 83 to 87, wherein —OS(═O)₂F is meta to the carbon atom linked to L¹.

Embodiment 90. The compound of Embodiment 89, wherein the compound of Formula (IV) is a compound of Formula (IVA):

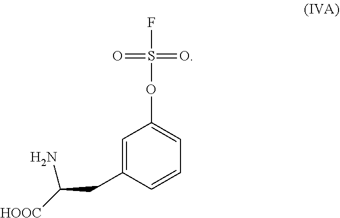 [SMILES: N[C@@H](Cc1cccc(OS(=O)(=O)F)c1)C(=O)O]

Embodiment 91. The compound of Embodiment 89, wherein the compound of Formula (IV) is a compound of Formula (IVB):

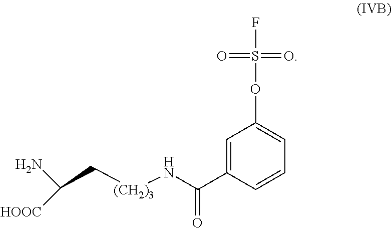

Embodiment 92. A protein comprising an unnatural amino acid, wherein the unnatural amino acid comprises a side chain of Formula (V):

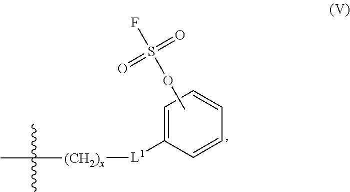

wherein —OS(═O)₂F is meta or ortho to the carbon atom linked to L¹; x is an integer from 1 to 8; and L¹ is a bond, substituted or unsubstituted alkylene, or substituted or unsubstituted heteroalkylene.

Embodiment 93. The protein of Embodiment 92, wherein x is an integer from 1 to 4.

Embodiment 94. The protein of Embodiment 92 or 93, wherein L¹ is a bond.

Embodiment 95. The protein of Embodiment 92 or 93, wherein L¹ is substituted or unsubstituted 2 to 6 membered heteroalkylene.

Embodiment 96. The protein of Embodiment 92 or 93, wherein L¹ is —NH—C(O)—(CH₂)_{y}— or —NH—C(O)—O—(CH₂)_{y}—, and y is an integer from 0 to 2.

Embodiment 97. The protein of any one of Embodiments 92 to 96, wherein —OS(═O)₂F is ortho to the carbon atom linked to L¹.

Embodiment 98. The protein of any one of Embodiments 92 to 96, wherein —OS(═O)₂F is meta to the carbon atom linked to L¹.

Embodiment 99. The protein of Embodiment 98, wherein the compound of Formula (V) is a compound of Formula (VA):

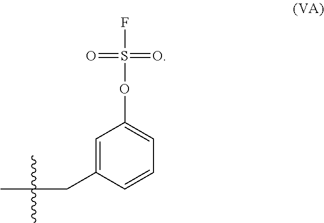 [SMILES: *Cc1cccc(OS(=O)(=O)F)c1]

Embodiment 100. The protein of Embodiment 98, wherein the compound of Formula (V) is a compound of Formula (VB):

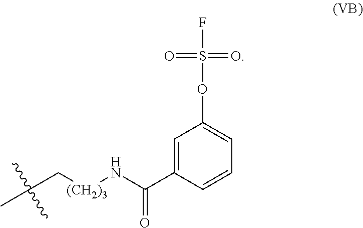

Embodiment 101. The protein of any one of Embodiments 92 to 100, wherein the protein is an antibody or an antibody variant.

Embodiment 102. The protein of Embodiment 101, wherein the antibody variant is a single-chain variable fragment, a single-domain antibody, an affibody, or an antigen-binding fragment.

Embodiment 103. The protein of any one of Embodiments 92 to 100, wherein the protein is a receptor protein.

Embodiment 104. A nucleic acid encoding the protein of any one of Embodiments 92 to 103.

Embodiment 105. A vector comprising the nucleic acid of Embodiment 104.

Embodiment 106. A biomolecule conjugate of Formula (VI):

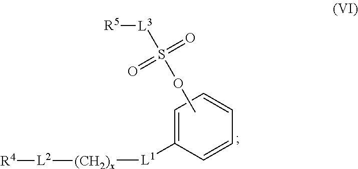

wherein: —OS(═O)₂L³R⁵ is meta or ortho to the carbon atom linked to L¹; R⁴ and R⁵ are each independently a peptidyl moiety, a carbohydrate moiety, or a nucleic acid moiety; L¹ is a bond, substituted or unsubstituted alkylene, or substituted or unsubstituted heteroalkylene; x is an integer from 1 to 8; L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 107. The biomolecule conjugate of Embodiment 106, wherein x is an integer from 1 to 4.

Embodiment 108. The biomolecule conjugate of Embodiment 106 or 107, wherein L¹ is a bond.

Embodiment 109. The biomolecule conjugate of Embodiment 106 or 107, wherein L¹ is substituted or unsubstituted 2 to 6 membered heteroalkylene.

Embodiment 110. The biomolecule conjugate of Embodiment 106 or 107, wherein L¹ is —NH—C(O)—(CH₂)_{y}— or —NH—C(O)—O—(CH₂)_{y}—, and y is an integer from 0 to 2.

Embodiment 111. The biomolecule conjugate of any one of Embodiments 106 to 110, wherein —OS(═O)₂L³R⁵ is ortho to the carbon atom linked to L¹.

Embodiment 112. The biomolecule conjugate of any one of Embodiments 106 to 110, wherein —OS(═O)₂L³R⁵ is meta to the carbon atom linked to L¹.

Embodiment 113. The biomolecule conjugate of Embodiment 112 having Formula (VIA):

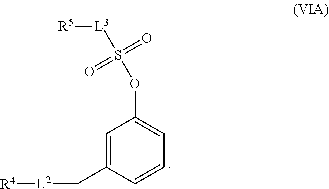

Embodiment 114. The biomolecule conjugate of Embodiment 112 having Formula (VIB):

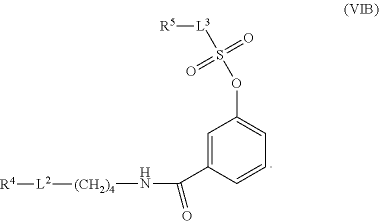

Embodiment 115. The biomolecule conjugate of any one of Embodiments 106 to 114, wherein R⁴ and R⁵ are each independently a peptidyl moiety.

Embodiment 116. The biomolecule conjugate of any one of Embodiments 106 to 115, wherein R⁵ is a peptidyl moiety comprising a lysine, histidine, or tyrosine bonded to L³.

Embodiment 117. The biomolecule conjugate of any one of Embodiments 106 to 116, wherein L³ is a bond.

Embodiment 118. The biomolecule conjugate of any one of claims 106 to 117, wherein L² is a bond.

Embodiment 119. The biomolecule conjugate of any one of Embodiments 106 to 118, wherein the peptidyl moiety of R⁴ comprises an antibody or an antibody variant; and the peptidyl moiety of R⁵ comprises a receptor protein.

Embodiment 120. The biomolecule conjugate of any one of Embodiments 114 to 117, wherein the peptidyl moiety of R⁴ comprises a receptor protein and the peptidyl moiety of R⁵ comprises an antibody or an antibody variant.

Embodiment 121. The biomolecule conjugate of Embodiment 119 or 120, wherein the antibody variant is a single-chain variable fragment, a single-domain antibody, an affibody, or an antigen-binding fragment.

Embodiment 122. A pyrrolysyl-tRNA synthetase comprising an amino acid sequence of SEQ ID NO:49.

Embodiment 123. A nucleic acid encoding the pyrrolysyl-tRNA synthetase of Embodiment 122.

Embodiment 124. A vector comprising the nucleic acid of Embodiment 123.

Embodiment 125. A complex comprising a pyrrolysyl-tRNA synthetase of Embodiment 122 and the compound of any one of Embodiments 83 to 91.

Embodiment 126. The complex of Embodiment 125, further comprising a tRNA^{Pyl}.

Embodiment 127. A cell comprising: (i) the compound of any one of Embodiments 83 to 91; (ii) the protein of any one of Embodiments 92 to 103; (iii) the nucleic acid of Embodiment 104 or 123; (iv) the vector of Embodiment 105 or 124; (v) the biomolecule conjugate of any one of Embodiments 106 to 121; or (vi) the complex of Embodiment 125 or 126.

Embodiment 128. The cell of Embodiment 127, wherein the cell is a bacterial cell or a mammalian cell.

Embodiment 129. A compound of Formula (I) or a stereoisomer thereof:

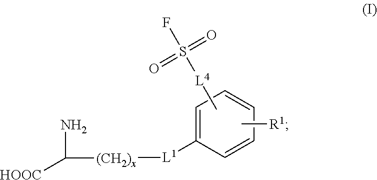

wherein: L⁴ is a bond or —O—; x is an integer from 1 to 8; and L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment 130. The compound of Embodiment 129, wherein -L⁴S(═O)₂F is para to the carbon atom linked to L¹.

Embodiment 131. The compound of Embodiment 129, wherein -L⁴S(═O)₂F is meta to the carbon atom linked to L.

Embodiment 132. The compound of Embodiment 129, wherein -L⁴S(═O)₂F is ortho to the carbon atom linked to L.

Embodiment 133. The compound of any one of Embodiments 129 to 132, wherein R¹ is para to -L⁴S(═O)₂F.

Embodiment 134. The compound of any one of Embodiments 129 to 132, wherein R₁ is meta to -L⁴S(═O)₂F.

Embodiment 135. The compound of any one of Embodiments 129 to 132, wherein R₁ is ortho to -L⁴S(═O)₂F.

Embodiment 136. The compound of any one of Embodiments 129 to 132, wherein the compound of Formula (I) is a compound of Formula (IA):

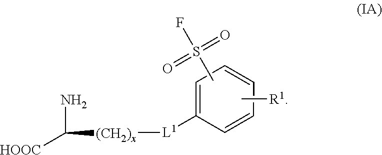

Embodiment 137. The compound of Embodiment 136, wherein the compound of Formula (IA) is a compound of Formula (IB):

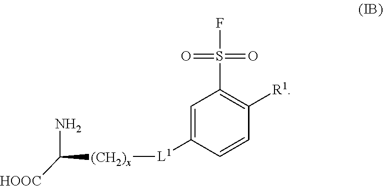

Embodiment 138. The compound of any one of Embodiments 129 to 135, wherein L⁴ is a bond.

Embodiment 139. The compound of any one of Embodiments 129 to 135, wherein L⁴ is —O—.

Embodiment 140. The compound of any one of Embodiments 129 to 139, wherein x is an integer from 1 to 4.

Embodiment 141. The compound of any one of Embodiments 129 to 140, wherein L¹ is a bond.

Embodiment 142. The compound of any one of Embodiments 129 to 140, wherein L¹ is substituted or unsubstituted 2 to 6 membered heteroalkylene.

Embodiment 143. The compound of any one of Embodiments 129 to 140, wherein L¹ is —NH—C(O)—(CH₂)_{y}— or —NH—C(O)—O—(CH₂)_{y}—, and y is an integer from 0 to 2.

Embodiment 144. The compound of any one of Embodiments 129 to 143, wherein R¹ is substituted or unsubstituted heteroalkyl.

Embodiment 145. The compound of any one of Embodiments 129 to 143, wherein R¹ is unsubstituted 2 to 8 membered heteroalkyl.

Embodiment 146. The compound of any one of Embodiments 129 to 143, wherein R¹ is —O—(CH₂)ₘCH₃, and m is an integer from 0 to 4.

Embodiment 147. The compound of any one of Embodiments 129 to 143, wherein R¹ is hydrogen.

Embodiment 148. The compound of Embodiment 129, wherein the compound of Formula (I) is a compound of Formula (IC) or a stereoisomer thereof:

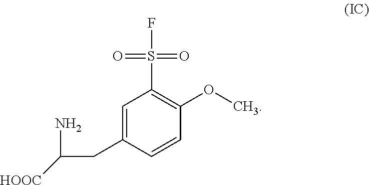 [SMILES: COc1ccc(CC(N)C(=O)O)cc1S(=O)(=O)F]

Embodiment 149. A compound of Formula (VII) or a stereoisomer thereof:

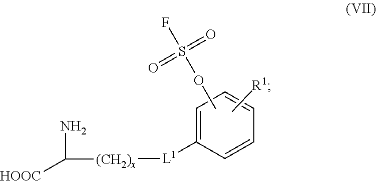

wherein: x is an integer from 1 to 8; L¹ is a bond, substituted or unsubstituted alkylene, or substituted or unsubstituted heteroalkylene; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR A, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted aryl, or substituted or unsubstituted heteroaryl.

Embodiment 150. The compound of Embodiment 149, wherein —OS(═O)₂F is ortho to the carbon atom linked to L¹.

Embodiment 151. The compound of Embodiment 149, wherein —OS(═O)₂F is meta to the carbon atom linked to L¹.

Embodiment 152. The compound of Embodiment 149, wherein —OS(═O)₂F is para to the carbon atom linked to L.

Embodiment 153. The compound of any one of Embodiments 149 to 152, wherein R¹ is ortho to —OS(═O)₂F.

Embodiment 154. The compound of any one of Embodiments 149 to 152, wherein R¹ is meta to —OS(═O)₂F.

Embodiment 155. The compound of any one of Embodiments 149 to 152, wherein R¹ is para to —OS(═O)₂F.

Embodiment 156. The compound of Embodiment 149, wherein the compound of Formula (VII) is a compound of Formula (VIIA):

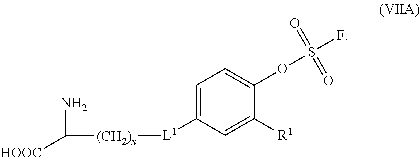

Embodiment 157. The compound of any one of Embodiments 149 to 156, wherein x is an integer from 1 to 4.

Embodiment 158. The compound of any one of Embodiments 149 to 157, wherein LU is a bond.

Embodiment 159. The compound of claim 149, wherein the compound of Formula (VII) is a compound of Formula (VIIB):

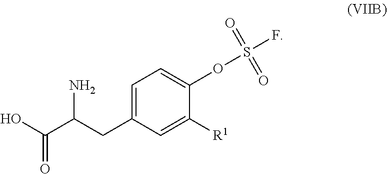

Embodiment 160. The compound of any one of Embodiments 149 to 159, wherein R¹ is halogen, —CX¹₃, —CHX¹₂, —CH₂X¹, —CN, —SOₙ₁R^{1A}, —SOᵥ₁NR^{1A}R^{1B}, —N(O)ₘ₁, —C(O)R^{1A}, —C(O)—OR^{1A}, —C(O)NR′^{A}R^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; nt is an integer from 0 to 4; m1 is 1 or 2; and v1 is 1 or 2.

Embodiment 161. The compound of Embodiment 160, wherein R¹ is —CX¹₃, —CHX¹₂, —CH₂X¹, —CN, —SOₙ₁R^{1A}, —SOᵥ₁NR^{1A}R^{1B}, —N(O)ₘ₁, —C(O)R^{1A}, —C(O)—OR^{1A}, or —C(O)NR^{1A}R^{1B}.

Embodiment 162. The compound of any one of Embodiments 149 to 161, wherein R^{1A} and R^{1B} are hydrogen.

Embodiment 163. The compound of any one of Embodiments 149 to 159, wherein R¹ is halogen.

Embodiment 164. The compound of Embodiment 149, wherein the compound of Formula (VII) is a compound of Formula (VIID) or a stereoisomer thereof:

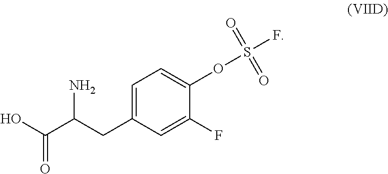 [SMILES: NC(Cc1ccc(OS(=O)(=O)F)c(F)c1)C(=O)O]

Embodiment 165. A protein comprising an unnatural amino acid, wherein the unnatural amino acid comprises a side chain of Formula (VIII):

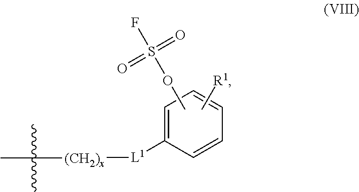

wherein: x is an integer from 1 to 8; L¹ is a bond, substituted or unsubstituted alkylene, or substituted or unsubstituted heteroalkylene; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted aryl, or substituted or unsubstituted heteroaryl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2.

Embodiment 166. The protein of Embodiment 165, wherein —OS(═O)₂F is ortho to the carbon atom linked to L¹.

Embodiment 167. The protein of Embodiment 165, wherein —OS(═O)₂F is meta to the carbon atom linked to L.

Embodiment 168. The protein of Embodiment 165, wherein —OS(═O)₂F is para to the carbon atom linked to L¹.

Embodiment 169. The protein of any one of Embodiments 165 to 168, wherein R¹ is ortho to —OS(═O)₂F.

Embodiment 170. The protein of any one of Embodiments 165 to 168, wherein R¹ is meta to —OS(═O)₂F.

Embodiment 171. The protein of any one of Embodiments 165 to 168, wherein R¹ is para to —OS(═O)₂F.

Embodiment 172. The protein of Embodiment 165, wherein the side chain of Formula (VIII) is a side chain of Formula (VIIIA):

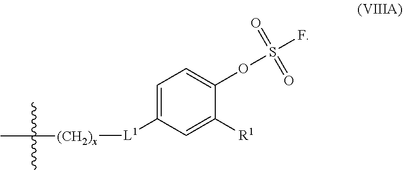

Embodiment 173. The protein of any one of Embodiments 165 to 172, wherein x is an integer from 1 to 4.

Embodiment 174. The protein of any one of Embodiments 165 to 173, wherein L¹ is a bond.

Embodiment 175. The protein of Embodiment 165, wherein the side chain of Formula (VIII) is a side chain of Formula (VIIIB):

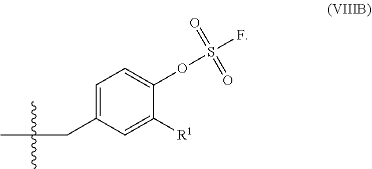

Embodiment 176. The protein of any one of Embodiments 165 to 175, wherein R¹ is halogen, —CX¹₃, —CHX¹₂, —CH₂X¹, —CN, —SOᵥ₁R^{1A}, —SOᵥ₁NR^{1A}R^{1B}, —N(O)ₘ₁, —C(O)R^{1A}, —C(O)—OR^{1A}, —C(O)NR^{1A}R^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment 177. The protein of Embodiment 176, wherein R¹ is —CX¹₃, —CHX¹₂, —CH₂X¹, —CN, —SOₙ₁R^{1A}, —SOᵥ₁NR^{1A}R^{1B}, —N(O)ₘ₁, —C(O)R^{1A}, —C(O)—OR^{1A}, or —C(O)NR^{1A}R^{1B}.

Embodiment 178. The protein of any one of Embodiments 165 to 177, wherein R^{1A} and R^{1B} are hydrogen.

Embodiment 179. The protein of any one of Embodiments 165 to 175, wherein R¹ is halogen.

Embodiment 180. The protein of Embodiment 179, wherein R¹ is —F.

Embodiment 181. The protein of any one of Embodiments 165 to 180, wherein the protein is an antibody or an antibody variant.

Embodiment 182. The protein of any one of Embodiments 165 to 180, wherein the protein is an antigen-binding fragment, a single-chain variable fragment, a single-domain antibody, or an affibody.

Embodiment 183. The protein of any one of Embodiments 165 to 180, wherein the protein is a receptor protein.

Embodiment 184. A nucleic acid encoding the protein of any one of Embodiments 14 to 29.

Embodiment 185. A vector comprising the nucleic acid of Embodiment 30.

Embodiment 186. A biomolecule conjugate comprising a first biomolecule moiety conjugated to a second biomolecule moiety through a bioconjugate linker, wherein the bioconjugate linker is Formula (X):

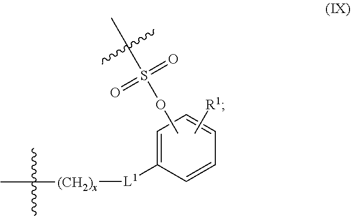

wherein: x is an integer from 1 to 8; L¹ is a bond, substituted or unsubstituted alkylene, or substituted or unsubstituted heteroalkylene; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, N(O)ₘ₁, —NR^{1A}R^{1B}, C(O)R^{1A}, —C(O)—OR^{1A}, C(O)NR^{1A}R^{1B}, OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted aryl, or substituted heteroaryl.

Embodiment 187. The biomolecule conjugate of Embodiment 186 having Formula (IXA):

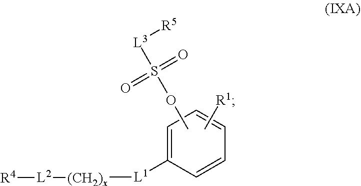

wherein: R² is the first biomolecule; R³ is the second biomolecule; L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is a bond, —N(R^{1A})—, —S—, —S(O)₂—, —O—, —C(O)—, —C(O)O—, —OC(O)—, —N(R^{3A})C(O)—, —C(O)N(R^{1A})—, —NR^{3A}C(O)NR^{3B}—, —NR^{3A}C(NH)NR^{3B}, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 188. The biomolecule conjugate of Embodiment 186 or 187, wherein —OS(═O)₂F is ortho to the carbon atom linked to L¹.

Embodiment 189. The biomolecule conjugate of Embodiment 186 or 187, wherein —OS(═O)₂F is meta to the carbon atom linked to L¹.

Embodiment 190. The biomolecule conjugate of Embodiment 186 or 187, wherein —OS(═O)₂F is para to the carbon atom linked to L.

Embodiment 191. The biomolecule conjugate of any one of Embodiments 186 to 190, wherein R¹ is ortho to —OS(═O)₂F.

Embodiment 192. The biomolecule conjugate of any one of Embodiments 186 to 190, wherein R¹ is meta to —OS(═O)₂F.

Embodiment 193. The biomolecule conjugate of any one of Embodiments 186 to 190, wherein R¹ is para to —OS(═O)₂F.

Embodiment 194. The biomolecule conjugate of Embodiment 187, wherein Formula (IXA) is a compound of Formula (XB):

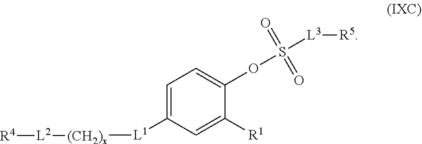

Embodiment 195. The biomolecule conjugate of any one of Embodiments 186 to 194, wherein x is an integer from 1 to 4.

Embodiment 196. The biomolecule conjugate of any one of Embodiments 186 to 195, wherein L¹ is a bond.

Embodiment 197. The biomolecule conjugate of any one of Embodiments 186 to 195, wherein L¹ is substituted or unsubstituted 2 to 6 membered heteroalkylene.

Embodiment 198. The biomolecule conjugate of any one of Embodiments 186 to 195, wherein L¹ is —NH—C(O)—(CH₂)_{y}— or —NH—C(O)—O—(CH₂)_{y}—, and y is an integer from 0 to 2.

Embodiment 199. The biomolecule conjugate of any one of Embodiments 187 to 198, wherein: L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-substituted or unsubstituted heteroarylene; L¹² is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl; L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹²-substituted or unsubstituted heteroarylene; and L¹³ is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl.

Embodiment 200. The biomolecule conjugate of any one of Embodiments 187 to 199, wherein L³ is a bond.

Embodiment 201. The biomolecule conjugate of any one of Embodiments 187 to 200, wherein L² is a bond.

Embodiment 202. The biomolecule conjugate of Embodiment 187, wherein the biomolecule conjugate of Formula (IXA) is a biomolecule conjugate of Formula (IXE), Formula (IXF), or Formula (IXG):

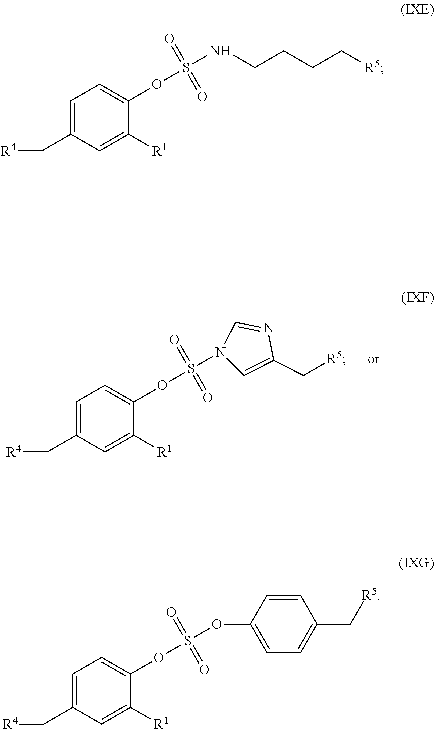

Embodiment 203. The biomolecule conjugate of any one of Embodiments 186 to 202, wherein R′ is halogen, —CX¹₃, —CHX¹₂, —CH₂X¹, —CN, —SOₙ₁R^{1A}, —SOᵥ₁NR^{1A}R^{1B}, —N(O)ₘ₁, —C(O)R^{1A}, —C(O)—OR^{1A}, —C(O)NR^{1A}R^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment 204. The biomolecule conjugate of Embodiment 203, wherein R¹ is —CX¹₃, —CHX¹₂, —CH₂X¹, —CN, —SOₙ₁R^{1A}, —SOᵥ₁NR^{1A}R^{1B}, N(O)ₘ₁, —C(O)R^{1A}, —C(O)—OR^{1A}, or —C(O)NR^{1A}R^{1B}.

Embodiment 205. The biomolecule conjugate of any one of Embodiments 186 to 204, wherein R^{1A} and R^{1B} are hydrogen.

Embodiment 206. The biomolecule conjugate of any one of Embodiments 186 to 202, wherein R¹ is halogen.

Embodiment 207. The biomolecule conjugate of Embodiment 206, wherein R¹ is —F.

Embodiment 208. The biomolecule conjugate of any one of Embodiments 187 to 207, wherein R⁴ and R⁵ are each independently a peptidyl moiety.

Embodiment 209. The biomolecule conjugate of Embodiment 208, wherein the peptidyl moiety of R⁴ comprises an antibody or an antibody variant; and the peptidyl moiety of R⁵ comprises a receptor protein.

Embodiment 210. The biomolecule conjugate of Embodiment 208, wherein the peptidyl moiety of R⁴ comprises a receptor protein and the peptidyl moiety of R⁵ comprises an antibody or an antibody variant.

Embodiment 211. The biomolecule conjugate of Embodiment 209 or 210, wherein the antibody variant is an antigen-binding fragment, a single-chain variable fragment, a single-domain antibody, or an affibody.

Embodiment 212. The biomolecule conjugate of any one of Embodiment 209 to 211, wherein the receptor protein is a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SiP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor.

Embodiment 213. The biomolecule conjugate of any one of Embodiments 209 to 211, wherein the receptor protein is a G protein-coupled receptor.

Embodiment 214. A complex comprising a pyrrolysyl-tRNA synthetase and the compound of any one of Embodiments 83-91 and 129-164.

Embodiment 215. The complex of Embodiment 214, wherein the pyrrolysyl-tRNA synthetase has an amino acid sequence with at least 90% sequence identity to SEQ ID NO:56.

Embodiment 216. The complex of Embodiment 214, wherein the pyrrolysyl-tRNA synthetase has an amino acid sequence as set forth in SEQ ID NO:57.

Embodiment 217. The complex of any one of Embodiments 214 to 216, further comprising a tRNA^{Pyl}.

Embodiment 218. The complex of Embodiment 217, wherein the tRNA^{Pyl} has the sequence as set forth in SEQ ID NO:59.

Embodiment 219. A cell comprising (i) the compound of any one of Embodiments 149 to 164; (ii) the protein of any one of Embodiments 165 to 183; (iii) the nucleic acid of Embodiment 184; (iv) the vector of Embodiment 185; (v) the biomolecule conjugate of any one of Embodiments 186 to 213; or (vi) the complex of any one of Embodiments 214 to 218.

Embodiment 220. The cell of Embodiment 219, wherein the cell is a bacterial cell or a mammalian cell.

Embodiment 221. The RNA-binding protein of any one of Embodiments 1 to 37, further comprising a detectable agent.

Embodiment 222. The RNA-binding protein of Embodiment 221, wherein the detectable agent is a radioisotope.

Embodiment 223. The biomolecule conjugate of any one of Embodiments 40-79, 106-121, and 186-213, further comprising a detectable agent.

Embodiment 224. The biomolecule conjugate of Embodiment 223, wherein the detectable agent is a radioisotope.

Embodiment 225. The protein of any one of Embodiments 92-103 and 165-183, further comprising a detectable agent.

Embodiment 226. The protein of Embodiment 225, wherein the detectable agent is a radioisotope.

Embodiment 227. The protein of Embodiment 103 or 183, wherein the receptor protein is a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SiP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor.

Embodiment 228. The protein of Embodiment 103 or 183, wherein the receptor protein is a G protein-coupled receptor.

Embodiment 229. The protein of Embodiment 103 or 183, wherein the receptor protein is a PD-L1 receptor or a PD-1 receptor.

Embodiment 230. The protein of Embodiment 103 or 183, wherein the receptor protein is an epidermal growth factor receptor (EGFR).

Embodiment 231. The protein of Embodiment 103 or 183, wherein the receptor protein is HER1, HER2, HER3, or HER4.

Embodiment 232. The biomolecule conjugate of any one of Embodiments 209 to 211, wherein the receptor protein is a PD-L1 receptor or a PD-1 receptor.

Embodiment 233. The biomolecule conjugate of any one of Embodiments 209 to 211, wherein the receptor protein is an epidermal growth factor receptor (EGFR).

Embodiment 234. The biomolecule conjugate of any one of Embodiments 209 to 211, wherein the EGFR receptor protein is HER1, HER2, HER3, or HER4.

### Embodiments N1-N104

Embodiment N1. A nanobody comprising an unnatural amino acid within CDR1, CDR2, or CDR3 of the nanobody; wherein the unnatural amino acid comprises a side chain of Formula (II):

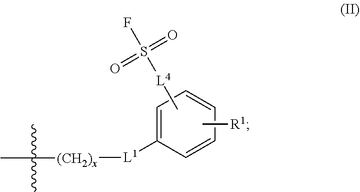

wherein: L⁴ is a bond or —O—; x is an integer from 1 to 8; L¹ is a bond, substituted or unsubstituted alkylene, or substituted or unsubstituted heteroalkylene; R′ is hydrogen, halogen, —CX′₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment N2. The nanobody of Embodiment N1, wherein the unnatural amino acid comprises a side chain of Formula (IE-A):

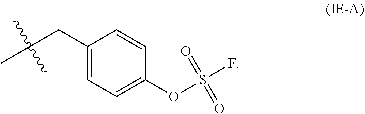 [SMILES: *Cc1ccc(OS(=O)(=O)F)cc1]

Embodiment N3. The nanobody of Embodiment N1, wherein the unnatural amino acid comprises a side chain of Formula (VA):

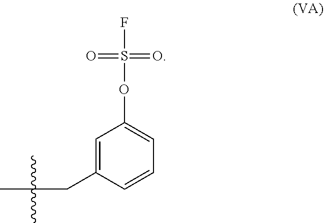 [SMILES: *Cc1cccc(OS(=O)(=O)F)c1]

Embodiment N4. The nanobody of Embodiment N1, wherein the unnatural amino acid comprises a side chain of Formula (VIIIC):

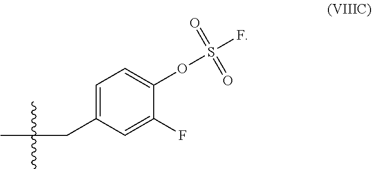 [SMILES: *Cc1ccc(OS(=O)(=O)F)c(F)c1]

Embodiment N5. The nanobody of Embodiment N1, wherein the unnatural amino acid comprises a side chain of Formula (VB):

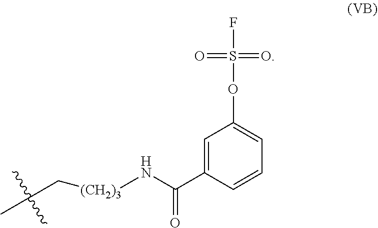

Embodiment N6. The nanobody of Embodiment N1, wherein the unnatural amino acid comprises a side chain of Formula (VB):

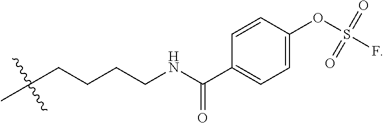 [SMILES: *CCCCNC(=O)c1ccc(OS(=O)(=O)F)cc1]

Embodiment N7. The nanobody of any one of Embodiments N1 to N6, wherein the nanobody comprises the unnatural amino acid within CDR1.

Embodiment N8. The nanobody of any one of Embodiments N1 to N6, wherein the nanobody comprises the unnatural amino acid within CDR2.

Embodiment N9. The nanobody of any one of Embodiments N1 to N6, wherein the nanobody comprises the unnatural amino acid within CDR3.

Embodiment N10. The nanobody of any one of Embodiments N1 to N9, wherein the nanobody comprises one unnatural amino acid.

Embodiment N11. The nanobody of any one of Embodiments N1 to N9, wherein the nanobody comprises two or three unnatural amino acids.

Embodiment N12. The nanobody of any one of Embodiments N1 to N11, comprising CDR1 as set forth in SEQ ID NO:67, CDR2 as set forth in SEQ ID NO:68; and CDR3 as set forth in SEQ ID NO:69.

Embodiment N13. The nanobody of Embodiment N12, wherein the unnatural amino acid is at a position corresponding to position 54 or 102 in SEQ ID NO:69.

Embodiment N14. The nanobody of Embodiment N1, comprising CDR1 as set forth in SEQ ID NO:67, CDR2 as set forth in SEQ ID NO:68; and CDR3 as set forth in SEQ ID NO:70.

Embodiment N15. The nanobody of Embodiment N1, comprising CDR1 as set forth in SEQ ID NO:67, CDR2 as set forth in SEQ ID NO:68; and CDR3 as set forth in SEQ ID NO:71.

Embodiment N16. The nanobody of any one of Embodiments N1 to N11, comprising CDR1 as set forth in SEQ ID NO:61, CDR2 as set forth in SEQ ID NO:62; and CDR3 as set forth in SEQ ID NO:63.

Embodiment N17 The nanobody of Embodiment N16, wherein the unnatural amino acid is at a position corresponding to position 10 in SEQ ID NO:63.

Embodiment N18. The nanobody of Embodiment N1, comprising CDR1 as set forth in SEQ ID NO:61, CDR2 as set forth in SEQ ID NO:62; and CDR3 as set forth in SEQ ID NO:64, 200, 202, 204, 206, 208, 210, or 212.

Embodiment N19. The nanobody of any one of Embodiments N1 to N11, comprising CDR1 as set forth in SEQ ID NO:75, CDR2 as set forth in SEQ ID NO:76; and CDR3 as set forth in SEQ ID NO:77.

Embodiment N20. The nanobody of Embodiment N19, wherein the unnatural amino acid is at a position corresponding to position 6 in SEQ ID NO:75.

Embodiment N21. The nanobody of Embodiment N1, comprising CDR1 as set forth in SEQ ID NO:78, CDR2 as set forth in SEQ ID NO:76, and CDR3 as set forth in SEQ ID NO:77.

Embodiment N22. The nanobody of any one of Embodiments N1 to N11, comprising CDR1 as set forth in SEQ ID NO:81, CDR2 as set forth in SEQ ID NO:82; and CDR3 as set forth in SEQ ID NO:83.

Embodiment N23. The nanobody of Embodiment N22, wherein the unnatural amino acid is at a position corresponding to position 5 or position 8 in SEQ ID NO:82; or the unnatural amino acid is at a position corresponding to 7 in SEQ ID NO:81.

Embodiment N24. The nanobody of Embodiment N1, comprising CDR1 as set forth in SEQ ID NO:81, CDR2 as set forth in SEQ ID NO:84 or SEQ ID NO:85; and CDR3 as set forth in SEQ ID NO: 83.

Embodiment N25. The nanobody of Embodiment N1, comprising CDR1 as set forth in SEQ ID NO:86, CDR2 as set forth in SEQ ID NO:82; and CDR3 as set forth in SEQ ID NO:83.

Embodiment N26. The nanobody of Embodiment N1, comprising CDR1 as set forth in SEQ ID NO:81, CDR2 as set forth in SEQ ID NO:87; and CDR3 as set forth in SEQ ID NO:83.

Embodiment N27. The nanobody of any one of Embodiments N1 to N11, comprising CDR1 as set forth in SEQ ID NO:93, CDR2 as set forth in SEQ ID NO:94; and CDR3 as set forth in SEQ ID NO:95.

Embodiment N28. The nanobody of Embodiment N27, wherein the unnatural amino acid is at a position corresponding to any one of positions 8 to 16 in SEQ ID NO:94; or the unnatural amino acid is at a position corresponding to position 5 or 6 in SEQ ID NO:95.

Embodiment N29. The nanobody of Embodiment N1, comprising CDR1 as set forth in SEQ ID NO:93, CDR2 as set forth in any one of SEQ ID NOS:96-102 and 105-113; and CDR3 as set forth in SEQ ID NO:95.

Embodiment N30. The nanobody of Embodiment N1, comprising CDR1 as set forth in SEQ ID NO:93, CDR2 as set forth in any one of SEQ ID NO:94; and CDR3 as set forth in any one of SEQ ID NOS:103, 104, 114, or 115.

Embodiment N31. The nanobody of Embodiment N1, comprising CDR1 as set forth in SEQ ID NO:155, CDR2 as set forth in any one of SEQ ID NO:156; and CDR3 as set forth in SEQ ID NO:181 or 182.

Embodiment N32. The nanobody of any one of Embodiment N1 to N11, comprising CDR1 as set forth in SEQ ID NO:215, CDR2 as set forth in SEQ ID NO:216, and CDR3 as set forth in SEQ ID NO:217.

Embodiment N33. The nanobody of Embodiment N32, wherein the unnatural amino acid is at a position corresponding to position 1, 3, 5, 6, or 8 in SEQ ID NO:215; or the unnatural amino acid is at a position corresponding to position 4, 5, 6, or 8 in SEQ ID NO:217.

Embodiment N34. The nanobody of Embodiment N1, comprising CDR1 as set forth in SEQ ID NO:218, 219, 220, 221, or 222, CDR2 as set forth in SEQ ID NO:216, or CDR3 as set forth in SEQ ID NO:217.

Embodiment N35. The nanobody of Embodiment N1, comprising CDR1 as set forth in SEQ ID NO:215, CDR2 as set forth in SEQ ID NO:216, and CDR3 as set forth in SEQ ID NO:223, 224, 225, or 226.

Embodiment N36. The nanobody of any one of Embodiments N1 to N11, comprising CDR1 as set forth in SEQ ID NO:240, CDR2 as set forth in SEQ ID NO:241, and CDR3 as set forth in SEQ ID NO:242.

Embodiment N37. The nanobody of Embodiment N36, wherein the unnatural amino acid is at a position corresponding to position 2, 4, 6, or 7 in SEQ ID NO:240; or the unnatural amino acid is at a position corresponding to position 2, 3, 4, or 5 in SEQ ID NO:241; or the unnatural amino acid is at a position corresponding to position 1, 6, 7, or 10 in SEQ ID NO:242.

Embodiment N38. The nanobody of Embodiment N1, comprising CDR1 as set forth in SEQ ID NO:243, 244, 245, or 246, CDR2 as set forth in SEQ ID NO:241, and CDR3 as set forth in SEQ ID NO:242.

Embodiment N39. The nanobody of Embodiment N1, comprising CDR1 as set forth in SEQ ID NO:240, CDR2 as set forth in SEQ ID NO:247, 248, 249, or 250, and CDR3 as set forth in SEQ ID NO:242.

Embodiment N40. The nanobody of Embodiment N1, comprising CDR1 as set forth in SEQ ID NO:240, CDR2 as set forth in SEQ ID NO:241, and CDR3 as set forth in SEQ ID NO:251, 252, 253, or 254.

Embodiment N41. The nanobody of any one of Embodiments N1 to N11, comprising CDR1 as set forth in SEQ ID NO:31, CDR2 as set forth in SEQ ID NO:32; and CDR3 as set forth in SEQ ID NO:33.

Embodiment N42. The nanobody of Embodiment N41, wherein the unnatural amino acid is at a position corresponding to position 5 or position 8 in SEQ ID NO:32.

Embodiment N43. The nanobody of any one of Embodiments N1 to N11, comprising CDR1 as set forth in SEQ ID NO:35, CDR2 as set forth in SEQ ID NO:36; and CDR3 as set forth in SEQ ID NO:37.

Embodiment N44. The nanobody of Embodiment N43, wherein the unnatural amino acid is at a position corresponding to position 4 in SEQ ID NO:37.

Embodiment N45. The nanobody of any one of Embodiments N1 to N11, comprising CDR1 as set forth in SEQ ID NO:39, CDR2 as set forth in SEQ ID NO:40; and CDR3 as set forth in SEQ ID NO:41.

Embodiment N46. The nanobody of Embodiment N45, wherein the unnatural amino acid is at a position corresponding to position 18 or position 19 in SEQ ID NO:41.

Embodiment N47. The nanobody of Embodiment N1, wherein the nanobody has an amino acid sequence with at least 90% sequence identity to any one of SEQ ID NOS:65, 73, 79, 88, 89, 90, 91, 116-127, 183-189, 199, 201, 203, 205, 207, 209, 211, 227-238, and 255-267; provided that the nanobody has 100% sequence identity with CDR1, CDR2, and CDR3 therein.

Embodiment N48. The nanobody of Embodiment N47, wherein the nanobody has an amino acid sequence with at least 95% sequence identity to any one of SEQ ID NOS:65, 73, 79, 88, 89, 90, 91, 116-127, 183-189, 199, 201, 203, 205, 207, 209, 211, 227-238, and 255-267; provided that the nanobody has 100% sequence identity with CDR1, CDR2, and CDR3 therein.

Embodiment N49. The nanobody of Embodiment N48, wherein the nanobody has an amino acid sequence as set forth in any one of SEQ ID NOS:65, 73, 79, 88, 89, 90, 91, 116-127, 183-189, 199, 201, 203, 205, 207, 209, 211, 227-238, and 255-267.

Embodiment N50. The nanobody of any one of Embodiments N1 to N49, provided that the nanobody is not nanobody 7D12; provided that the nanobody has less than 100% sequence identity with CDR1 as set forth in SEQ ID NO:155, CDR2 as set forth in SEQ ID NO:156, or CDR3 as set forth in SEQ ID NO:157; or provided that the nanobody having CDR1 as set forth in SEQ ID NO:155, CDR2 as set forth in SEQ ID NO:156, and CDR3 as set forth in SEQ ID NO:157 does not contain an FSY unnatural amino acid in CDR1, CDR2, or CDR3 and does not contain an FSK unnatural amino acid in CDR1, CDR2, or CDR3.

Embodiment N51. The nanobody of any one of Embodiments N1 to N50, provided that the nanobody is not nanobody KN035; provided that the nanobody has less than 100% sequence identity to CDR1, CDR2, and CDR3 in SEQ ID NO: 177 or SEQ ID NO:178; or provided that the nanobody has less than 100% sequence identity to SEQ ID NO:177 or SEQ ID NO:178.

Embodiment N52. The nanobody of any one of Embodiments 1 to 51, further comprising a detectable agent.

Embodiment N53. The nanobody of Embodiment N52, wherein the detectable agent is a radioisotope.

Embodiment N54. The nanobody of any one of Embodiments N1 to N53, further comprising a therapeutic agent.

Embodiment N55. A fusion protein comprising a first protein and a second protein, wherein the first protein is a first nanobody of any one of Embodiments N1 to N54.

Embodiment N56. The fusion protein of Embodiment N55, wherein the first protein is covalently bonded to the second protein via a glycine-serine peptide linker.

Embodiment N57. The fusion protein of Embodiment N55 or N56, wherein the second protein is an antigen-binding fragment, a single-chain variable fragment, a second nanobody, or an affibody.

Embodiment N58. The fusion protein of Embodiment N55 or N56, wherein the second protein has at least 90% sequence identity to the amino acid sequence of SEQ ID NO:219, SEQ ID NO:137, SEQ ID NO:138, SEQ ID NO: 139, SEQ ID NO:180, SEQ ID NO: 192, SEQ ID NO:193, SEQ ID NO:194, SEQ ID NO:195, SEQ ID NO:196, SEQ ID NO: 197, or SEQ ID NO:198.

Embodiment N59. The fusion protein of Embodiment N58, wherein the second protein has at least 95% sequence identity to the amino acid sequence of SEQ ID NO:219, SEQ ID NO:137, SEQ ID NO:138, SEQ ID NO:139, SEQ ID NO:180, SEQ ID NO: 192, SEQ ID NO:193, SEQ ID NO:194, SEQ ID NO:195, SEQ ID NO:196, SEQ ID NO: 197, or SEQ ID NO:198.

Embodiment N60. The fusion protein of Embodiment N59, wherein the second protein is as set forth in SEQ ID NO:219, SEQ ID NO:137, SEQ ID NO:138, SEQ ID NO:139, SEQ ID NO:180, SEQ ID NO:192, SEQ ID NO:193, SEQ ID NO:194, SEQ ID NO: 195, SEQ ID NO:196, SEQ ID NO:197, or SEQ ID NO:198.

Embodiment N61. The fusion protein of any one of Embodiments N58 to N60, wherein the first nanobody comprises CDR1 as set forth in SEQ ID NO:155, CDR2 as set forth in SEQ ID NO:156, and CDR3 as set forth in SEQ ID NO:158 or 159.

Embodiment N62. The fusion protein of any one of Embodiments N58 to N60, wherein the nanobody comprises CDR1 as set forth in SEQ ID NO:155, CDR2 as set forth in SEQ ID NO:156, and CDR3 as set forth in SEQ ID NO: 183 or 184.

Embodiment N63. The fusion protein of any one of Embodiments N58 to N60, wherein the nanobody comprises CDR1 as set forth in SEQ ID NO:93, CDR2 as set forth in any one of SEQ ID NOS:96-102 and 105-113, and CDR3 as set forth in SEQ ID NO:95.

Embodiment N64. The fusion protein of any one of Embodiments N58 to N60, wherein the nanobody comprises CDR1 as set forth in SEQ ID NO:93, CDR2 as set forth in SEQ ID NO:94, and CDR3 as set forth in SEQ ID NO: 103, 104, 114, or 115.

Embodiment N65. The fusion protein of any one of Embodiments N58 to N60, wherein the nanobody comprises CDR1 as set forth in SEQ ID NO:67, CDR2 as set forth in SEQ ID NO:70, and CDR3 as set forth in SEQ ID NO:69.

Embodiment N66. The fusion protein of any one of Embodiments N58 to N60, wherein the nanobody comprises CDR1 as set forth in SEQ ID NO:67, CDR2 as set forth in SEQ ID NO:68, and CDR3 as set forth in SEQ ID NO:71.

Embodiment N67. The fusion protein of Embodiment N55 or N56, wherein the first nanobody comprises: (i) CDR1 as set forth in SEQ ID NO:155, CDR2 as set forth in SEQ ID NO:156, and CDR3 as set forth in SEQ ID NO:157; or (ii) CDR1 as set forth in SEQ ID NO:155, CDR2 as set forth in SEQ ID NO: 156, and CDR3 as set forth in SEQ ID NO: 158 or 159; and wherein the second protein comprises a second nanobody, wherein the second nanobody comprises: (a) CDR1 as set forth in SEQ ID NO:67, CDR2 as set forth in SEQ ID NO:68, and CDR3 as set forth in SEQ ID NO:69; (b) CDR1 as set forth in SEQ ID NO:67, CDR2 as set forth in SEQ ID NO:70, and CDR3 as set forth in SEQ ID NO:69; (c) CDR1 as set forth in SEQ ID NO:67, CDR2 as set forth in SEQ ID NO:68, and CDR3 as set forth in SEQ ID NO:71; provided that the first nanobody is not (i) when the second nanobody is (a).

Embodiment N68. The fusion protein of Embodiment N55 or N56, wherein the first protein is the first nanobody and the second protein is a second nanobody, wherein the first nanobody and the second nanobody are selected from the group consisting of: (i) CDR1 as set forth in SEQ ID NO:61, CDR2 as set forth in SEQ ID NO:62, and CDR3 as set forth in SEQ ID NO:63; (ii) CDR1 as set forth in SEQ ID NO:61, CDR2 as set forth in SEQ ID NO:62, and CDR3 as set forth in SEQ ID NO: 64; (iii) CDR1 as set forth in SEQ ID NO:61, CDR2 as set forth in SEQ ID NO:62, and CDR3 as set forth in SEQ ID NO:200; (iv) CDR1 as set forth in SEQ ID NO:61, CDR2 as set forth in SEQ ID NO:62, and CDR3 as set forth in SEQ ID NO:202; (v) CDR1 as set forth in SEQ ID NO:61, CDR2 as set forth in SEQ ID NO:62, and CDR3 as set forth in SEQ ID NO:204; (vi) CDR1 as set forth in SEQ ID NO:61, CDR2 as set forth in SEQ ID NO:62, and CDR3 as set forth in SEQ ID NO:206; (vii) CDR1 as set forth in SEQ ID NO:61, CDR2 as set forth in SEQ ID NO:62, and CDR3 as set forth in SEQ ID NO:208; (viii) CDR1 as set forth in SEQ ID NO:61, CDR2 as set forth in SEQ ID NO:62, and CDR3 as set forth in SEQ ID NO:210; and (ix) CDR1 as set forth in SEQ ID NO:61, CDR2 as set forth in SEQ ID NO:62, and CDR3 as set forth in SEQ ID NO:212; provided that the first nanobody and the second nanobody are not both (i).

Embodiment N69. The fusion protein of Embodiment N55, wherein the fusion protein has at least 90% sequence identity to the amino acid sequence of any one of SEQ ID NOS:130, 131, 132, 133, 135, 136, 141, 143, 144, 146, 148, 150, 151, and 153; provided that the nanobody has 100% sequence identity with CDR1, CDR2, and CDR3 therein.

Embodiment N70. The fusion protein of Embodiment N69, wherein the fusion protein has at least 95% sequence identity to the amino acid sequence of any one of SEQ ID NOS:130, 131, 132, 133, 135, 136, 141, 143, 144, 146, 148, 150, 151, and 153; provided that the nanobody has 100% sequence identity with CDR1, CDR2, and CDR3 therein.

Embodiment N71. The fusion protein of Embodiment N70, wherein the fusion protein has the amino acid sequence of any one of SEQ ID NOS:130, 131, 132, 133, 135, 136, 141, 143, 144, 146, 148, 150, 151, and 153.

Embodiment N72. The fusion protein of any one of Embodiments N54 to N70, further comprising a detectable agent.

Embodiment N73. The fusion protein of Embodiment N71, wherein the detectable agent is a radioisotope.

Embodiment N74. The fusion protein of any one of Embodiments N55 to N73, further comprising a therapeutic agent.

Embodiment N75. A protein comprising an unnatural amino acid within CDR-L1, CDR-L2, CDR-L3, CDR-H1, CDR-H2, or CDR-H3, wherein the protein is an antigen-binding fragment, a single-chain variable fragment, or an antibody

Embodiment N76. The protein of Embodiment N75, wherein the unnatural amino acid comprises a side chain of Formula (IE-A):

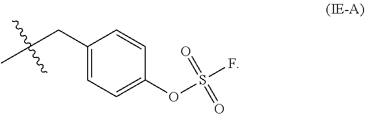 [SMILES: *Cc1ccc(OS(=O)(=O)F)cc1]

Embodiment N77. The protein of Embodiment N75, wherein the unnatural amino acid comprises a side chain of Formula (VA):

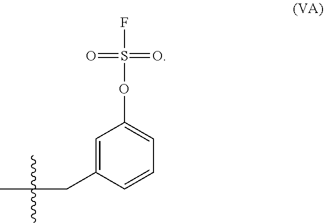 [SMILES: *Cc1cccc(OS(=O)(=O)F)c1]

Embodiment N78. The protein of Embodiment N75, wherein the unnatural amino acid comprises a side chain of Formula (VIIIC):

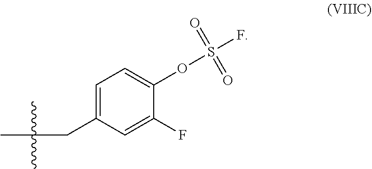 [SMILES: *Cc1ccc(OS(=O)(=O)F)c(F)c1]

Embodiment N79. The protein of Embodiment N75, wherein the unnatural amino acid comprises a side chain of Formula (VB):

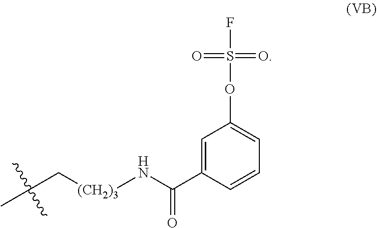

Embodiment N80. The protein of Embodiment N75, wherein the unnatural amino acid comprises a side chain of Formula (VB):

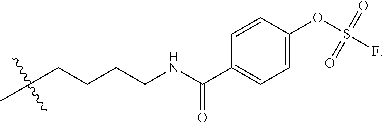 [SMILES: *CCCCNC(=O)c1ccc(OS(=O)(=O)F)cc1]

Embodiment N81. The protein of any one of Embodiments N75 to N80, wherein the protein is an antigen-binding fragment.

Embodiment N82. The protein of Embodiment N81, wherein the antigen-binding fragment is a trastuzumab antigen-binding fragment having CDR-L1 as set forth in SEQ ID NO:163, CDR-L2 as set forth in SEQ ID NO:165, CDR-L3 as set forth in SEQ ID NO:165, CDR-H1 as set forth in SEQ ID NO:171, CDR-H2 as set forth in SEQ ID NO:172, and CDR-H3 as set forth in SEQ ID NO:173.

Embodiment N83. The protein of Embodiment N81, wherein the protein is an antigen-binding fragment having CDR-L1 as set forth in SEQ ID NO:163, CDR-L2 as set forth in SEQ ID NO:165, CDR-L3 as set forth in SEQ ID NO: 166 or 167, CDR-H1 as set forth in SEQ ID NO:171, CDR-H2 as set forth in SEQ ID NO:172, and CDR-H3 as set forth in SEQ ID NO:173.

Embodiment N84. A protein having at least 90% sequence identity to any one of SEQ ID NOS:2, 3, 4, 22, 26, 29, 174, 176, 179, 180, 192, 193, 194, 195, 196, 197, 198, and 199, provided that the protein comprises the unnatural amino acid therein.

Embodiment N85. The protein of Embodiment N84 having at least 95% sequence identity to any one of SEQ ID NOS:2, 3, 4, 22, 26, 29, 174, 176, 179, 180, 192, 193, 194, 195, 196, 197, 198, and 199, provided that the protein comprises the unnatural amino acid therein.

Embodiment N86. The protein of Embodiment N85, having any one of SEQ ID NOS:2, 3, 4, 22, 26, 29, 174, 176, 179, 180, 192, 193, 194, 195, 196, 197, 198, and 199.

Embodiment N87. The protein of any one of Embodiments N75 to N86, further comprising a detectable agent.

Embodiment N88. The protein of Embodiment N87, wherein the detectable agent is a radioisotope.

Embodiment N89. The protein of any one of Embodiments N75 to N88, further comprising a therapeutic agent.

Embodiment N90. A pharmaceutical composition comprising: (i) a pharmaceutically acceptable excipient, and (ii) the nanobody of any one of Embodiments N1 to N54, the fusion protein of any one of Embodiments N55 to N74, or the protein of any one of Embodiments N75 to N89.

Embodiment N91. A method of detecting cancer in a patient in need thereof, the method comprising administering to the patient an effective amount of the nanobody of Embodiment N52 or N53, the fusion protein of Embodiment N72 or N73, or the protein of Embodiment N87 or N88, thereby detecting cancer in the patient.

Embodiment N92. The method of Embodiment N91, comprising administering to the patient the effective amount of the nanobody.

Embodiment N93. A method of monitoring cancer progression or cancer treatment in a patient in need thereof, the method comprising administering to the patient an effective amount of the nanobody of Embodiment N52 or N53, the fusion protein of Embodiment N72 or N73, or the protein of Embodiment N87 or N88 at a first time point, thereby detecting cancer in the patient; and administering to the patient an effective amount of the nanobody of Embodiment N52 or N53, the fusion protein of Embodiment N72 or N73, or the protein of Embodiment N87 or N88, respectively, at a second time point later than the first time point, thereby monitoring the cancer progression or cancer treatment.

Embodiment N94. The method of Embodiment N93, comprising administering to the patient the effective amount of the nanobody at the first time point, and administering to the patient the effective amount of the nanobody at the second time point later than the first time point.

Embodiment N95. The method of any one of Embodiments N91 to N94, wherein the cancer expresses HER2 or wherein the cancer overexpresses HER2 relative to a control.

Embodiment N96. The method of any one of Embodiments N91 to N94, wherein the cancer expresses mesothelin or wherein the cancer overexpresses mesothelin relative to a control.

Embodiment N97. The method of any one of Embodiments N91 to N95, wherein the nanobody comprises CDR1 as set forth in SEQ ID NO:67, CDR2 as set forth in SEQ ID NO:70, and CDR3 as set forth in SEQ ID NO:69.

Embodiment N98. The method of any one of Embodiments N91 to N95, wherein the nanobody comprises CDR1 as set forth in SEQ ID NO:67, CDR2 as set forth in SEQ ID NO:68, and CDR3 as set forth in SEQ ID NO:71.

Embodiment N99. The method of any one of Embodiments N91-N94 and N96, wherein the nanobody comprises CDR1 as set forth in SEQ ID NO:218, 219, 220, 221, or 222, CDR2 as set forth in SEQ ID NO:216, or CDR3 as set forth in SEQ ID NO:217; or wherein the nanobody comprises CDR1 as set forth in SEQ ID NO:215, CDR2 as set forth in SEQ ID NO:216, and CDR3 as set forth in SEQ ID NO:223, 224, 225, or 226.

Embodiment N100. The method of any one of Embodiments N91-N94 and N96, wherein the nanobody comprises CDR1 as set forth in SEQ ID NO:243, 244, 245, or 246, CDR2 as set forth in SEQ ID NO:241, and CDR3 as set forth in SEQ ID NO:242; or wherein the nanobody comprises CDR1 as set forth in SEQ ID NO:240, CDR2 as set forth in SEQ ID NO:247, 248, 249, or 250, and CDR3 as set forth in SEQ ID NO:242; or wherein the nanobody comprises CDR1 as set forth in SEQ ID NO:240, CDR2 as set forth in SEQ ID NO:241, and CDR3 as set forth in SEQ ID NO:251, 252, 253, or 254.

Embodiment N101. The method of any one of Embodiments N91 to N100, wherein the detectable agent is a positron-emitting radioisotope.

Embodiment N102. The method of Embodiment N101, wherein the positron-emitting radioisotope is ¹¹C, ¹³N, ¹⁵O, ¹⁸F, ⁶⁴Cu, ⁶⁸Ga, ⁷⁸Br, ⁸²Rb, ⁸⁶Y, ⁸⁹Zr, ⁹⁰Y, ²²Na, ²⁶Al, ⁴⁰K, ⁸³Sr, or ¹²⁴I.

Embodiment N103. The method of any one of Embodiments N91 to N100, wherein the detectable agent is an alpha-emitting radioisotope.

Embodiment N104. The method of Embodiment N103, wherein the alpha-emitting radioisotope is ²¹¹At, ²²⁷Th, ²²⁵Ac, ²²³Ra, ²¹³Bi, or ²¹²Bi.

### EXAMPLES

The following examples are intended to further illustrate certain embodiments of the disclosure. The examples are put forth so as to provide one of ordinary skill in the art and are not intended to limit its scope.

### Example 1

RNA-binding proteins (RBPs) regulate almost all aspects of RNA molecules inside cells, from pre-mRNA splicing, 3′ tail processing, to RNA modification, translation, degradation, and localization. (Ref 1). These regulatory roles of RBPs are essential for cells and organisms to maintain normal physiological status. Functional defects of RBPs could be the causes of many disorders, such as neurodegeneration and cancer. (Refs 2-3). Numerous monogenic diseases have mutations enriched in RNA-binding regions, suggesting they arise from altered RNA binding. (Ref 4). Inside cells, most RBPs have a specific or multiple subcellular localizations, where they could interact with different sets of target RNA molecules through competing or collaborating with other RBPs. (Ref 5). In addition, hundreds of RBPs have recently been uncovered that lack conventional RNA-binding domains (RBDs) and many bind RNA with intrinsically disordered regions. (Ref 6). Some RBPs may even be inversely regulated by RNA. To understand the complex regulatory mechanisms and emerging novel aspects of RBPs, it is critical to identify the interactions between RBPs and their endogenous target RNA molecules under physiological conditions, ideally with nucleotide resolution and amino acid resolution.

Interactions of RBP and RNA in vivo are generally dynamic, transient, and weak. (Ref 7). To preserve RBP-RNA interactions for identification, the most widely used approach is nucleoside-based UV crosslinking, in which a nucleoside base produces radicals in response to UV-irradiation to crosslink with proximal amino acid residues. (Refs 8-9). Various technologies based on this mechanism have been developed to crosslink protein-RNA. Together with immunoprecipitation (IP) and high-throughput sequencing techniques, RNA targets of many RBPs can be determined, which has largely expanded our understanding of RNA regulations. (Refs 6, 10-15). However, nucleoside-based UV crosslinking has strong nucleotide bias toward uridine, making it difficult to study RBPs lacking uridines in the target RNA regions. (Refs. 9, 11, 16). UV crosslinking could only capture interaction events during the short UV-illumination window, and the short half-life of nucleoside radicals decreases detection sensitivity. Due to poor tissue penetrance, UV crosslinking cannot be applied to intact nontransparent animals for in vivo studies. More importantly, as the crosslinking moiety is generated on RNA and radicals crosslink with amino acid residues nonspecifically, nucleoside-based UV crosslinking cannot afford amino acid resolution for RBPs. For instance, RNA targets of different RNA-binding regions of a RBP cannot be resolved. Moreover, nonspecific crosslinking of RNA to protein residues makes it infeasible to rationally design and engineer protein-RNA complexes with precise covalent linkages. There is a need in the art to identify and develop compounds and methods to crosslink proteins and RNA.

Latent bioreactive unnatural amino acids (Uaas) have been genetically incorporated into proteins in live cells, which react with specific natural amino acid residues via proximity-enabled reactivity. (Refs 17-18). These latent bioreactive Uaas permit the selective chemical crosslinking of protein with protein both in vitro and in vivo, which has enabled a broad range of new applications such as pinpointing ligand-receptor binding, capturing elusive protein-protein interactions, and developing covalent protein drugs. (Refs 19-21).

Here we expanded the ability of latent bioreactive Uaas to enable genetically encoded chemical crosslinking of proteins (e.g., CRISPR proteins) with RNA in vivo. We genetically incorporated two latent bioreactive Uaas, fluorosulfate-L-tyrosine (FSY) and o-sulfonyl fluoride-O-methyltyrosine (SFY), that were able to react with all four RNA nucleotides via proximity-enabled SuFEx only when the RNA bound to RNA-binding proteins, irreversibly capturing target RNA on RNA-binding proteins in both E. coli and mammalian cells. By applying GECX-RNA to the RNA chaperon Hfq in E. coli, we demonstrated RNA identification with protein residue specificity in vivo. In addition, through genetic incorporation of SFY into the YTH domain in mammalian cells, we devised a new, antibody-free method for in vivo identification of M-methyladenosine (m6A) sites on mRNA with single-nucleotide resolution. GECX-RNA now enables the study of protein-RNA interactions in vivo with single-nucleotide resolution for RNAs as well as amino acid specificity for proteins. Furthermore, selectively targeting RNA via proximity-enabled reactivity will open new avenues for generation of covalent protein-RNA complex for research and therapeutic applications.

### Results

Developing GECX-RNA to crosslink RNA to RNA-binding proteins via genetically encoding latent bioreactive Uaas

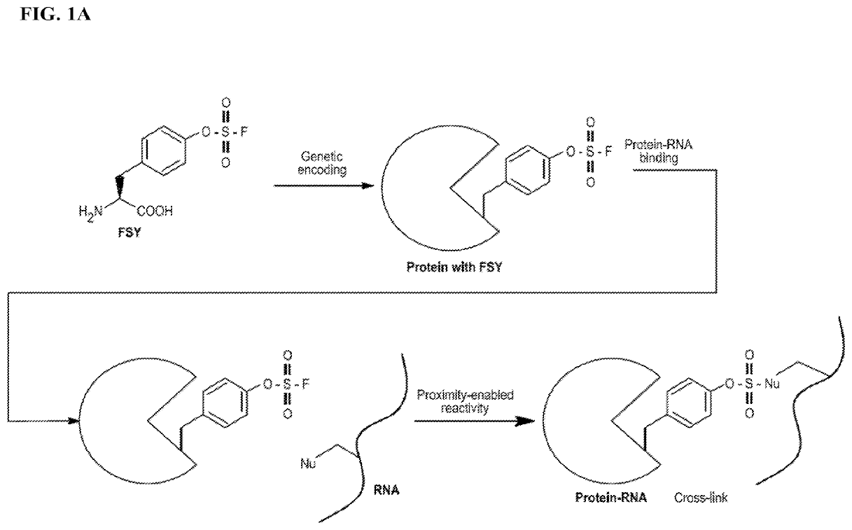

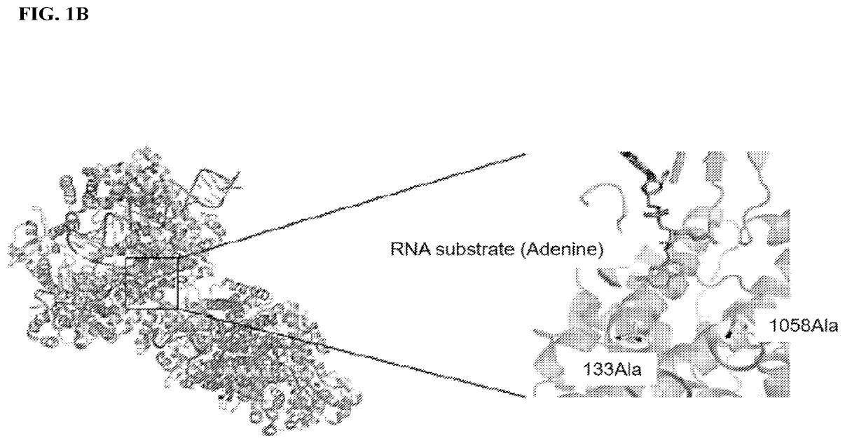

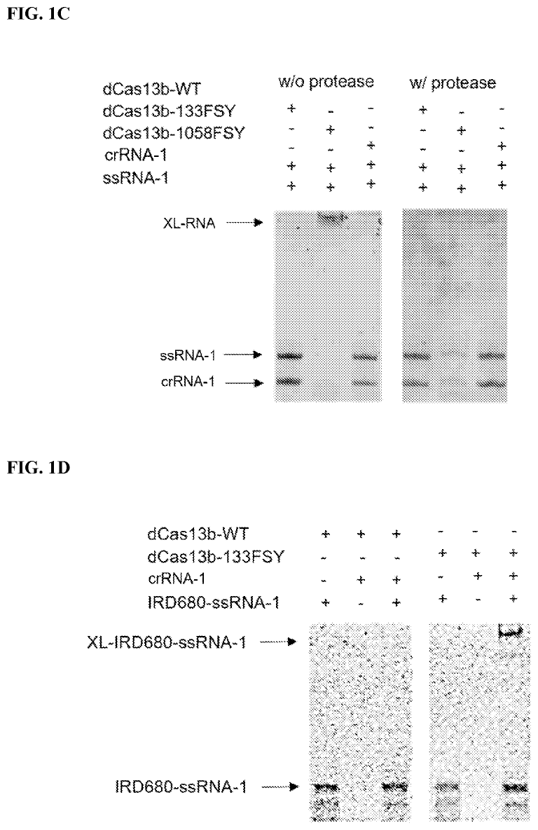

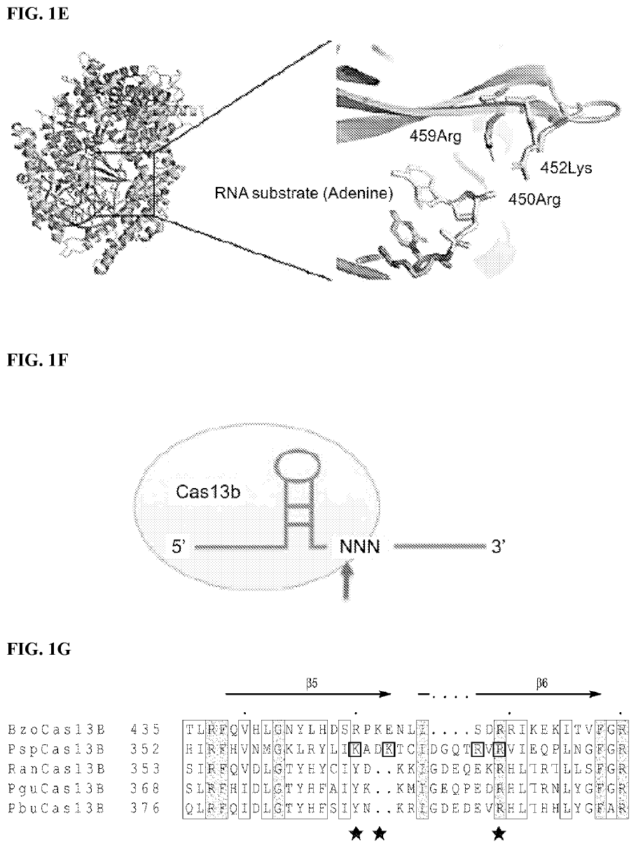

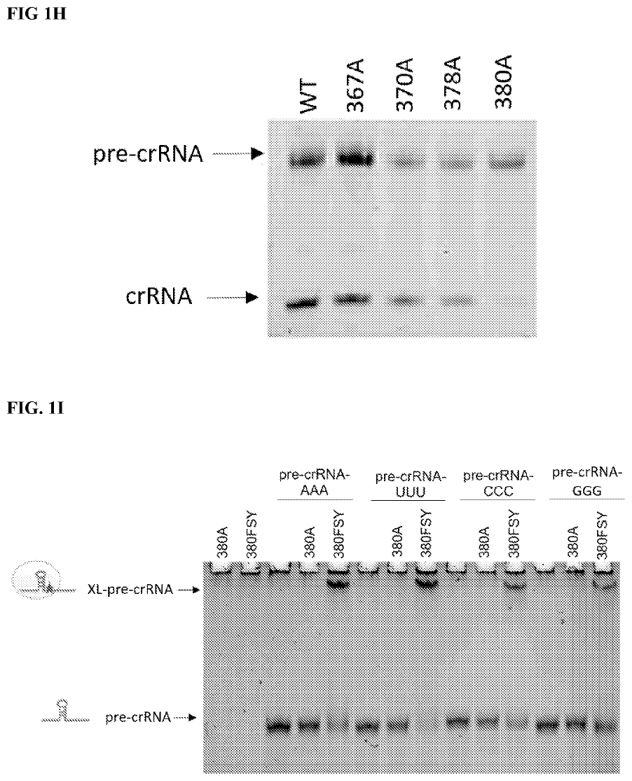

The latent bioreactive Uaa fluorosulfate-L-tyrosine (FSY) has recently been genetically incorporated into proteins in E. coli and mammalian cells via a newly evolved orthogonal tRNA^{Pyl}/FSYRS pair. (Ref 22). Through proximity-enabled SuFEx reaction, the incorporated FSY specifically crosslinks with proximal Lys, His, and Tyr side chains, forming covalent linkages within or between proteins in vivo. (Ref 22). When there is no nucleophiles placed in close proximity, the aryl fluorosulfate of FSY remains intact in proteins and inside cells. Based on such selective reactivity, we hypothesized that SuFEx reactions could potentially target the nucleophilic 2′-hydroxyl group of ribose or amine groups of base in proximal nucleotides, thus forming covalent linkages between a protein and its bound RNA (FIG. 1A).

To test this hypothesis, we used Cas13 as the model protein to examine whether FSY-incorporated RNA-binding proteins could crosslink with interacting target RNA. Cas13b is a class 2 type VI RNA-guided RNA-targeting CRISPR-Cas effector. (Refs 24-26). Catalytically inactive Cas13b from Prevotella sp. P5-125 (dPsCas13b) maintains targeted RNA binding activity and could only bind to targeted RNA through guidance of CRISPR RNA (crRNA). (Ref 26). Based on the crystal structure of homologous Cas13a-crRNA-target RNA ternary complex (FIG. 1B), we first prepared the catalytically inactive Cas13b (dPsCas13b) mutant by mutating His133 and His1058 of PsCas13b into alanine (FIG. 1B). (Refs 25, 27). These two His are conserved catalytic residues responsible for RNA backbone cleavage. We thus incorporated FSY separately into position 133 and 1058 of dPsCas13b, reasoning that at these positions FSY side chain should aim at the 2′-OH group of RNA backbone (FIG. 1B). The wildtype dPsCas13b (dCas13-WT) and two FSY-incorporated dPsCas13b mutant proteins (dCas13b-133FSY, dCas13b-1058FSY) were expressed and purified, and then incubated with crRNA (crRNA-1) and target RNA (ssRNA-1). After incubation, the samples were examined on denaturing Urea-PAGE. RNA with crosslinked dCas13b proteins would run slower than RNA alone on Urea-PAGE. Indeed, we observed protein-RNA crosslinked bands for samples containing dCas13b-133FSY, whereas no such band for samples containing dCas13b-WT or dCas13b-1058FSY (FIG. 1C), indicating that FSY-incorporated at His133 of dCas13b crosslinks with RNA.

To further validate whether the crosslinked bands were the crosslinking products of FSY-incorporated protein and RNA, we treated the crosslinked product of dCas13b-133FSY with protease K and re-analyzed with denaturing Urea-PAGE (FIG. 1C). Concomitant with the disappearance of the crosslinked bands, the target and guide RNA bands reappeared, indicating they were captured by dCas13b-133FSY protein. Apart from the target ssRNA, the excess guide crRNA was also covalently crosslinked by dCas13b-133FSY, which is consistent with the collateral RNA cleavage activity of Cas13b in vitro. (Ref 28). In addition, we verified the crosslinked band contained the target ssRNA by using 5′-fluorescently labeled target ssRNA (IRD680-ssRNA-1), which showed fluorescence in the crosslinked band (FIG. 1D). Moreover, in the absence of the guide crRNA, no crosslinked protein-RNA bands were detected (FIG. 1D). Since the presence of guide RNA is necessary for Cas13b to bind and cleave RNA, this result indicate that the covalent crosslink of RNA to dCas13b-133FSY depends on RNA binding, which places RNA in close proximity to the FSY located at the catalytic site 133.

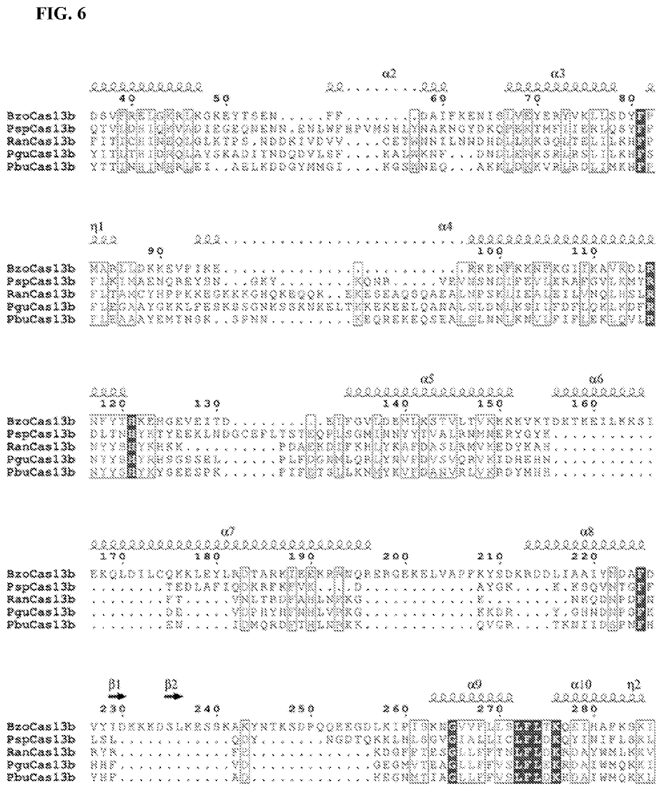

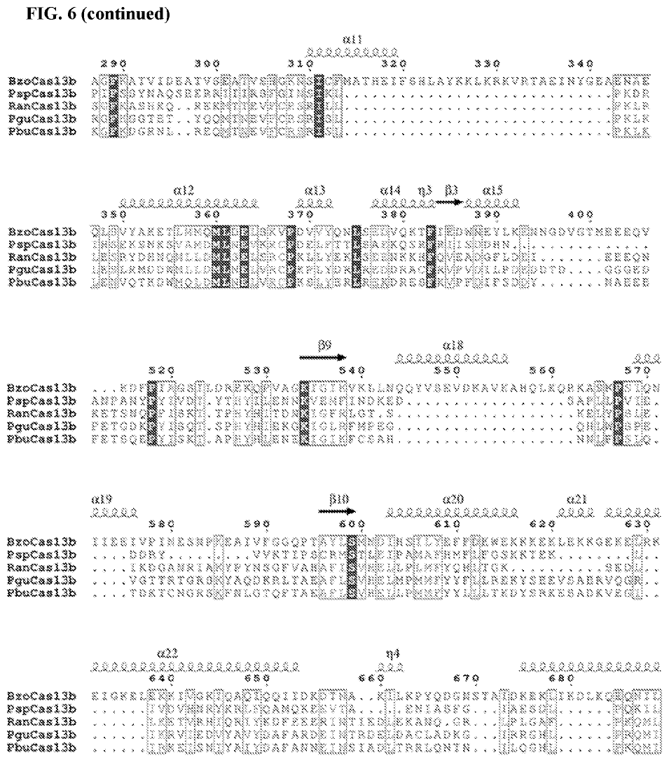

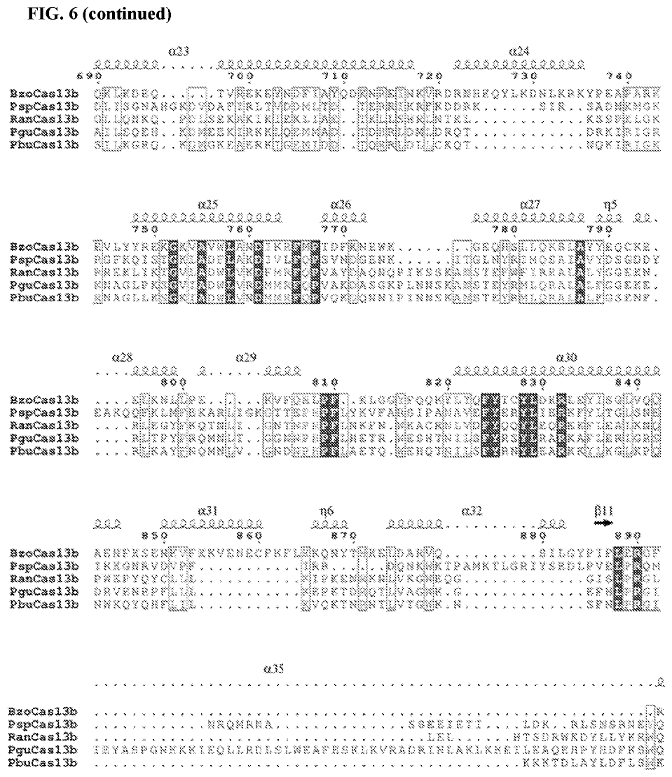

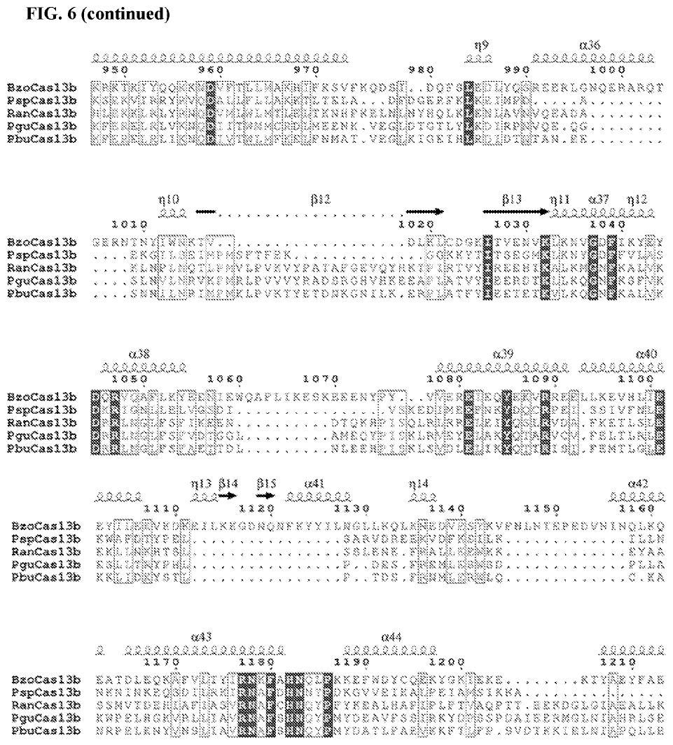

We next investigated whether FSY could crosslink all four RNA nucleotides via targeting the 2′ hydroxyl group on the ribose. To do so, we used another cleavage feature of Cas13b protein. In addition to the cleavage site responsible for target RNA cleavage, Cas13b protein also contains another cleavage site that site specifically cleaves precursor crRNA (pre-crRNA) into mature crRNA. (Refs 23-24, 28). Based on the crystal structure of Bergeyella zoohelcum Cas13b-crRNA binary complex (FIG. 1E), we reasoned that positively charged amino acids on β-sheets 5 and 6 are involved in pre-crRNA cleavage because they are in close contact with two cleavage nucleotides located directly 3′-downstream of the hairpin repeat region of pre-crRNA (FIG. 1F). (Ref 23). To identify the positively charged amino acids of PsCas13b involved in pre-crRNA cleavage, we mutated K367, K370, R378, and R380 of dPsCas13b into alanine, respectively, based on homology alignment (FIG. 1G, FIG. 6). A pre-crRNA containing a 38-nt sequence at 3′-downstream of the hairpin repeat region of pre-crRNA was used as the cleavage target. We found that dPsCas13b-WT was active in pre-crRNA cleavage (FIG. 1H). For dPsCas13b alanine mutants, K367A, K370A, and R378A mutants were still active in pre-crRNA cleavage, while R380A mutant abolished pre-crRNA cleavage (FIG. 1H). Considering R380 site is conserved among Cas13b proteins from different species (FIG. 1G, FIG. 6) and its homologous R459 is in close contact with the backbone of the two cleavage nucleotides in the crystal structure of Bergeyella zoohelcum Cas13b-crRNA binary complex, R380 of dPsCas13b must play an important role in pre-crRNA cleavage. Thus, we mutated R380 of dPsCas13b into FSY, and incubated dPsCas13b-380 mutant proteins separately with four pre-crRNAs that had different nucleotide compositions at cleavage sites of pre-crRNA (FIG. 1I). As expected, no crosslinking was observed for dPsCas13b-380Ala incubations. Gratifyingly, dPsCas13b-380FSY protein crosslinked with pre-crRNAs with all four nucleotide compositions at the cleavage site (FIG. 1I), indicating an unbiased nucleotide cross-linking reactivity of FSY when targeting the ribose 2′-hydroxyl.

These results together demonstrated that FSY incorporated into RNA-binding proteins could crosslink with RNA bound in proximity in vitro, representing the first Uaa-enabled, Genetically Encoded Chemical cross-linking of RNA (GECX-RNA) in an unbiased nucleotide-crosslinking manner.

GECX-RNA enables crosslinking of target RNA to RNA-binding proteins with amino acid specificity in E. coli

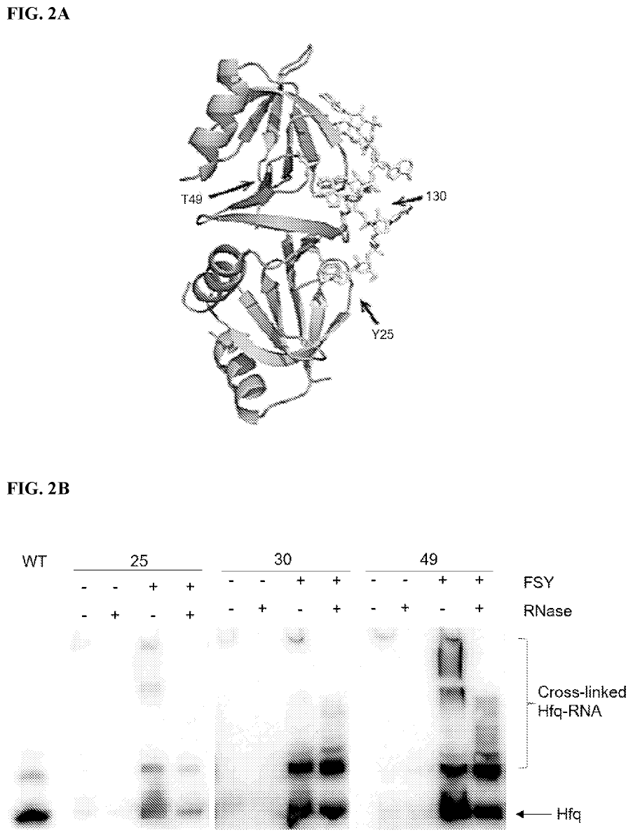

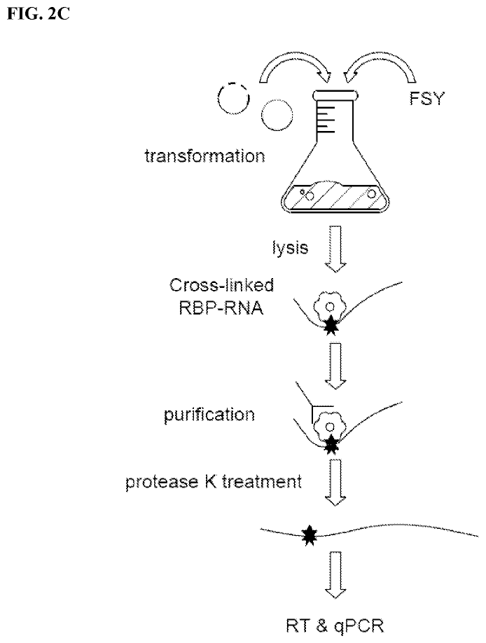

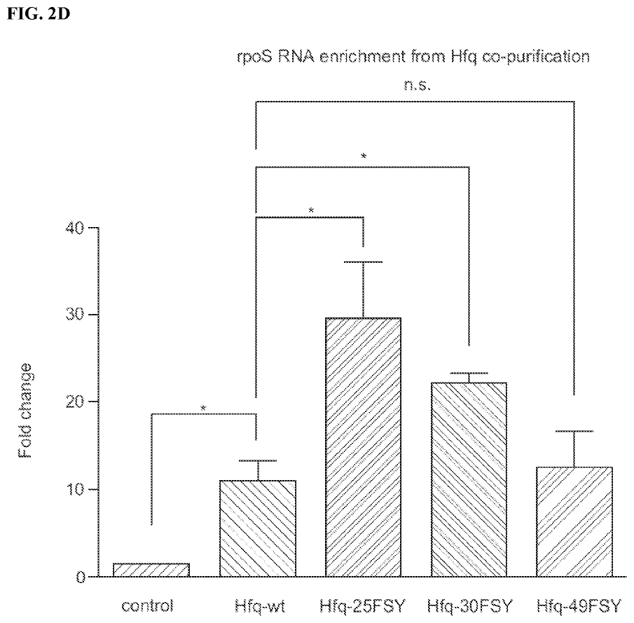

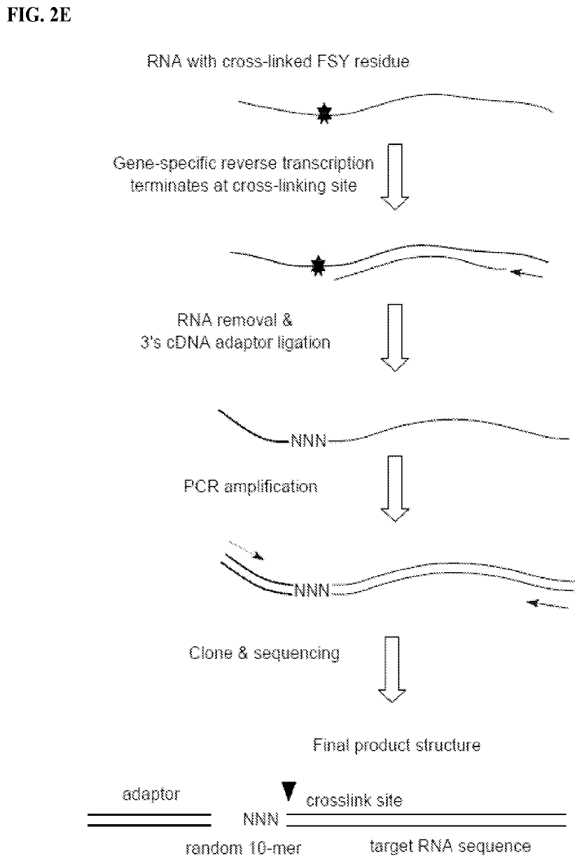

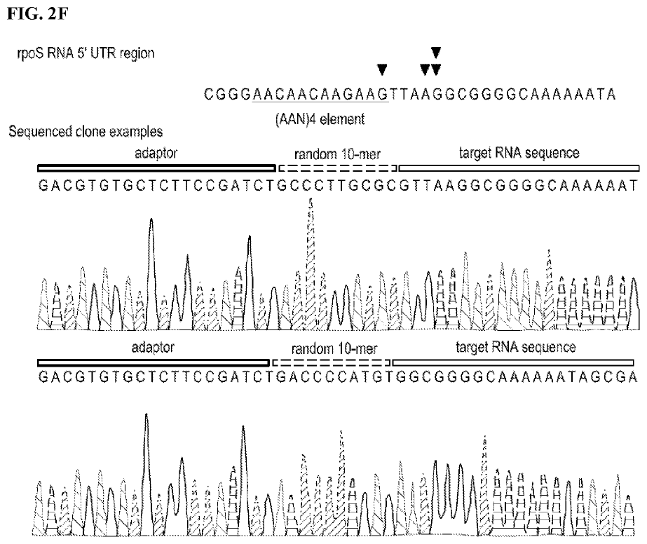

To test if GECX-RNA was suitable for capturing interactions of RNA-binding proteins with target RNAs in vivo, we first examined if it could be applied for crosslinking the endogenous RNA targets of bacterial RNA-binding proteins, host factor required for Qβ replication (Hfq), in E. coli. Hfq is a widely conserved bacterial RNA chaperone, interacting with hundreds of sRNAs and more than one thousand mRNAs in Gram-negative bacteria, such as E. coli. (Refs 29-32). In light of the structure of E. coli Hfq binding to target RNA, we introduced FSY into the binding interface at sites 25, 30, and 49 of E. coli Hfq protein (FIG. 2A), respectively. (Ref 33). The E. coli Hfq-WT, Hfq-25FSY, Hfq-30FSY, or Hfq-49FSY protein was separately expressed in E. coli DH10B strain. After culturing, Western blot analysis of cell lysates showed crosslinking bands in all three samples expressing FSY-incorporated Hfq, but not Hfq-WT (FIG. 2B). These crosslinking bands disappeared when samples were treated with RNase, indicating that the crosslinking products were Hfq-FSY proteins crosslinked with RNAs (FIG. 2B).

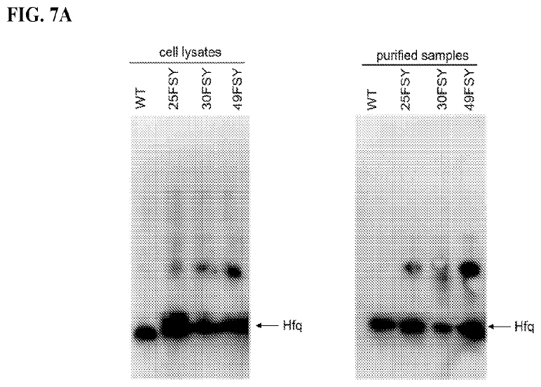

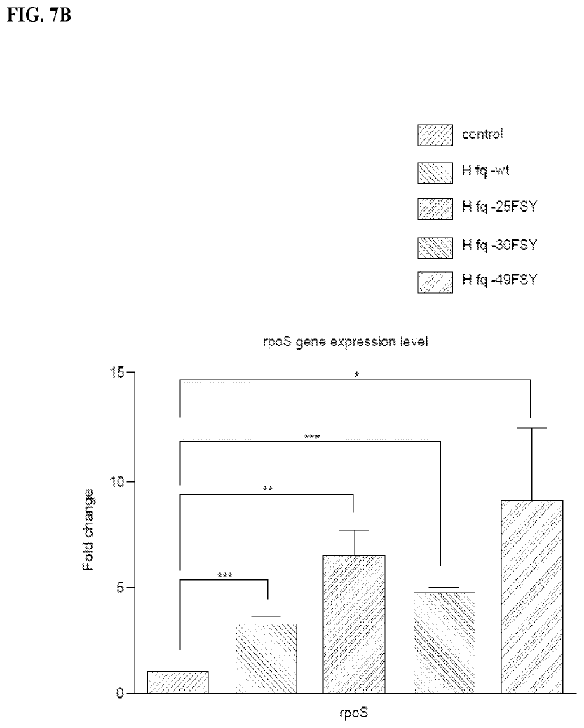

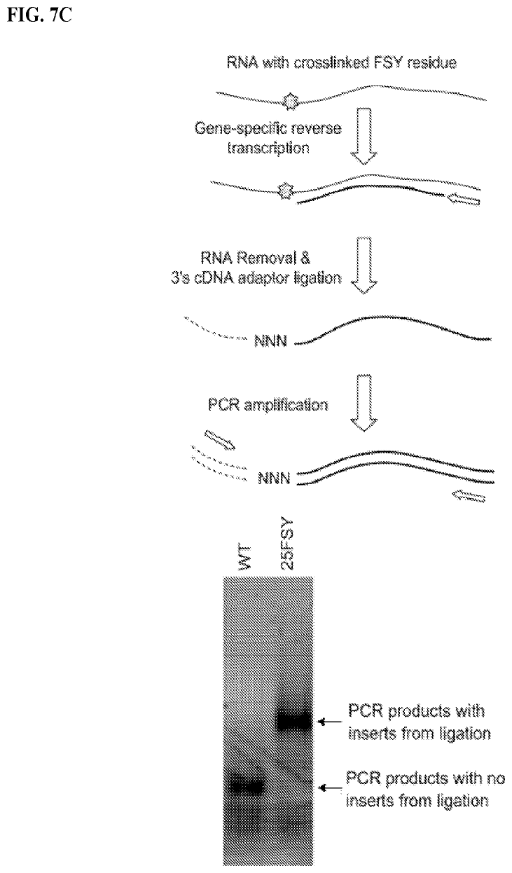

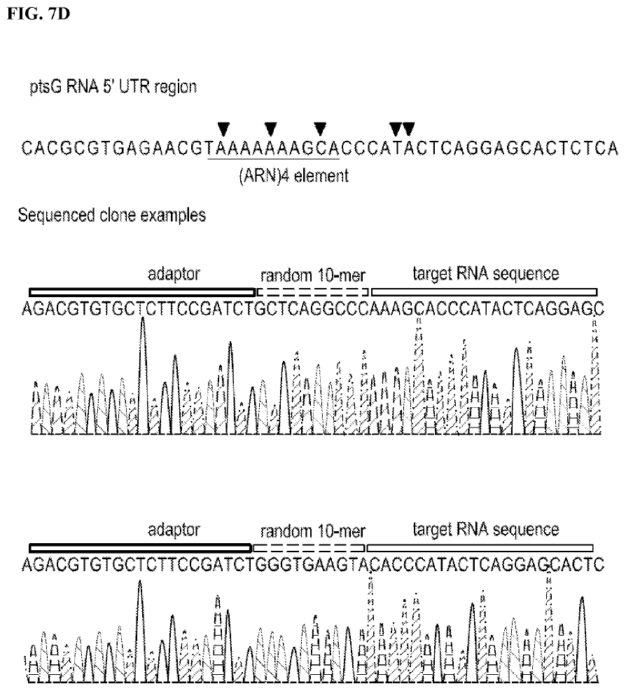

To determine whether the RNAs crosslinked with the Hfq-FSY proteins were endogenous target RNAs of Hfq, we expressed and purified Hfq proteins from E. coli, and examined the abundances of rpoS RNA, one known Hfq target RNA, co-purified with different Hfq proteins (FIG. 2C, FIG. 7A). rpoS RNA showed similar up-regulations in both Hfq-WT and Hfq-FSY expressing cells, indicating that FSY-incorporated Hfq proteins were functional in E. coli cells (FIG. 7B). More importantly, RT-qPCR analysis showed that, in comparison with the RNA samples co-purified with Hfq-WT, those co-purified with Hfq-25FSY and Hfq-30FSY were more enriched in rpoS RNA (FIG. 2D), demonstrating that FSY-incorporation in Hfq could specifically crosslink and enrich target RNAs in E. coli cells.

A key potential of GECX-RNA is to capture and identify RNA with amino acid specificity of RNA-binding proteins, since the chemical crosslinker is site-selectively introduced into RNA-binding proteins and not in RNA. To demonstrate this ability, we combined GECX-RNA with immunoprecipitation (GRIP) to determine RNA crosslinking sites of specific amino acid positions on RNA-binding proteins (FIG. 2E). In general, RNA-binding proteins with FSY incorporated at a desired site will be expressed in cells to allow RNA crosslinking in vivo. Following cell lysis, RNA-binding proteins-FSY together with crosslinked RNA will be purified and digested with protease K to remove RNA-binding proteins and release the crosslinked RNAs. The RNAs will be reverse transcribed with gene-specific primer targeting a downstream region of crosslinked positions, which will terminate at the crosslinking site due to the crosslinked FSY residue. After removal of RNA, cDNA will be ligated to an adaptor at the 3′ end and then amplified with PCR. The PCR products will be sequenced, from which the Uaa-induced crosslinking sites on target RNA will be identified at the ligation sites of PCR amplicons.

Site 25 of Hfq protein has been proposed to contact with an (AAN)4 element on rpoS RNA for regulation, but all evidence is from either in vitro experiments or indirect in vivo indications. (Ref 34). To directly detect the binding nucleotides of Tyr25 of Hfq protein in E. coli cells, we applied GRIP on Hfq-25FSY expressed in E. coli cells using gene-specific RT and PCR primers for rpoS. As expected, in the final PCR product Hfq-WT sample had no insertion, while Hfq-25FSY sample had distinct insertion (FIG. 7C), indicating that Hfq-25FSY but not Hfq-WT captured rpoS RNA in vivo. Sanger sequencing of PCR products from the Hfq-25FSY sample revealed that reverse transcription was terminated at the (AAN)4 element or its immediate 3′ region (FIG. 2F). These results indicate that site 25 of Hfq protein contacted with the (AAN)4 element on rpoS RNA in E. coli cells, providing in vivo evidence to support previous in vitro studies. (Ref 34).

We further applied GRIP with gene-specific RT and PCR primers for another Hfq target mRNA, ptsG. (Refs 35-36). Previously, it has been predicted that the ARN motifs in the UTR region of ptsG interact with Hfq proteins in E. coli cells, but there is no direct evidence of which domain of Hfq protein binds with ptsG mRNA in vivo. (Ref 35). After sanger sequencing of PCR products from the Hfq-25FSY sample, similar to the result for rpoS gene, we also identified that reverse transcripts of ptsG mRNAs terminated at the (ARN)4 element or its immediate 3′ region (FIG. 7D), indicating that site 25 of Hfq directly contact the (ARN)4 element of the ptsG RNA. These results from rpoS and ptsG mRNAs presented the first direct in vivo experimental evidence of site 25 of Hfq binding with ARN elements on target RNAs, demonstrating the power of GECX-RNA in probing in vivo protein-RNA interactions with amino acid specificity.

### GECX-RNA Enables Specific Crosslinking of Target RNA to RNA-Binding Proteins in Mammalian Cells

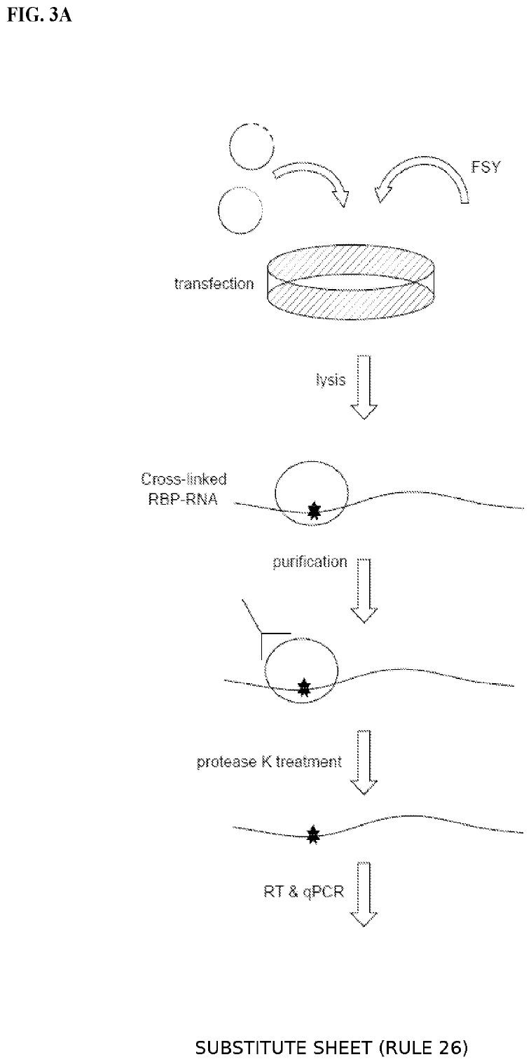

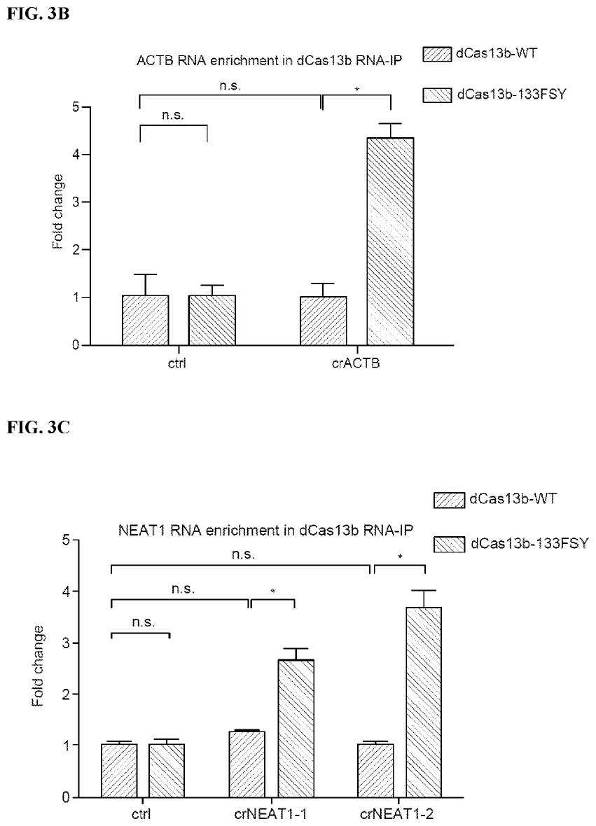

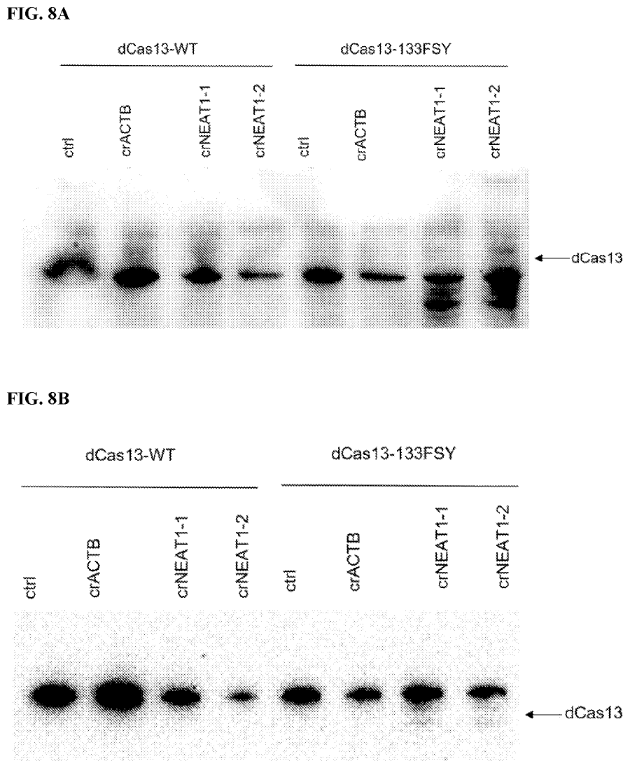

We next tested whether GECX-RNA could work in mammalian cells and enable crosslinking of RNA-binding proteins with target RNA specifically. dCas13b proteins bind to specific endogenous RNA targets via the guidance of crRNA in mammalian systems, which should serve as an excellent system to test GECX-RNA for crosslinking and specificity. (Refs 25-26). We therefore co-transfected plasmids expressing wild type dPsCas13b (WT) or FSY-incorporated dPsCas13b (dPsCas13b-133FSY) together with crRNA expressing plasmids into HEK293T cells. Western blot analysis confirmed the successful expression of dPsCas13b-WT and dPsCas13b-133FSY protein (FIG. 8). They were purified through immunoprecipitation and digested by protease K to release the bound RNA (FIG. 3A). The RNAs were then reverse transcribed and quantified by qPCR using primers specific for the same gene as the guide crRNA. In the first case where target RNA was ACTB (FIG. 3B), in the negative control without introducing guide crRNA, dPsCas13b-133FSY had no enrichment of target RNA over dPsCas13b-WT, indicating that dPsCas13b-133FSY did not crosslink the target RNA when it was not bound. In contrast, in the presence of crRNA, dPsCas13b-WT still did not enrich target RNA over the control, while dPsCas13b-133FSY showed 4 fold enrichment over dPsCas13b-WT. Similar results were also obtained for NEAT1 RNA using two guide crRNAs targeting different regions of NEAT1 RNA, respectively (FIG. 3B). These results demonstrate that GECX-RNA could crosslink and enrich target RNA in mammalian cells, and the crosslinking was dependent on RNA binding with RNA-binding proteins to achieve target RNA specificity.

### Genetically Encoding SFY in Mammalian Cells to Expand SuFEx-Based Crosslinking in Cells

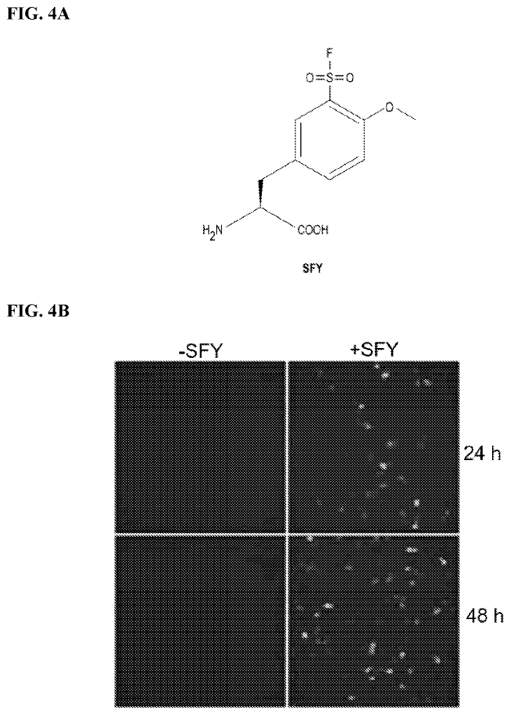

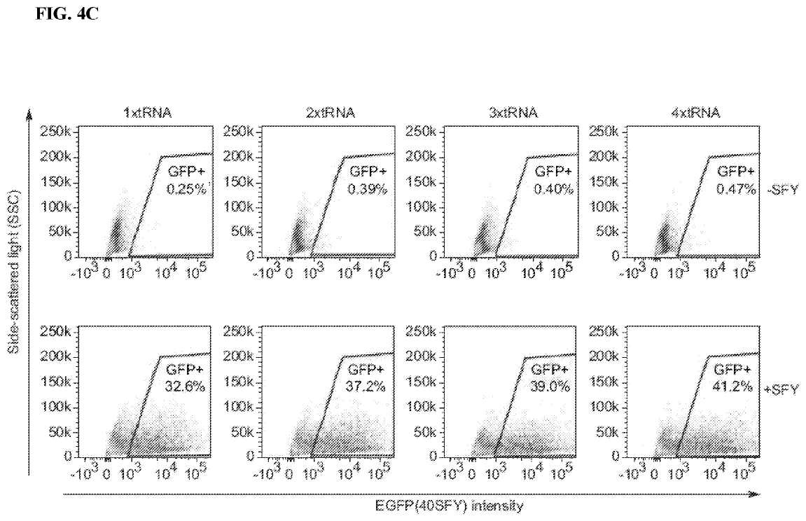

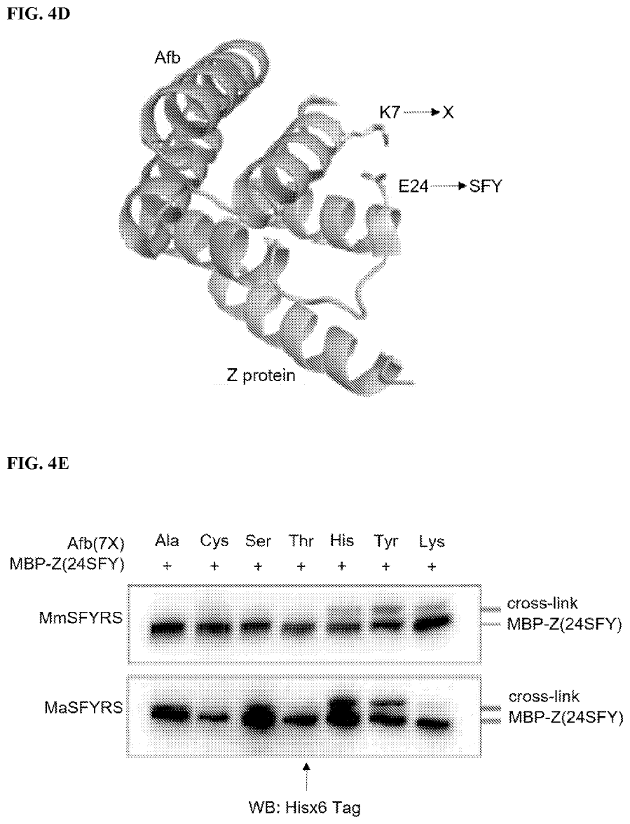

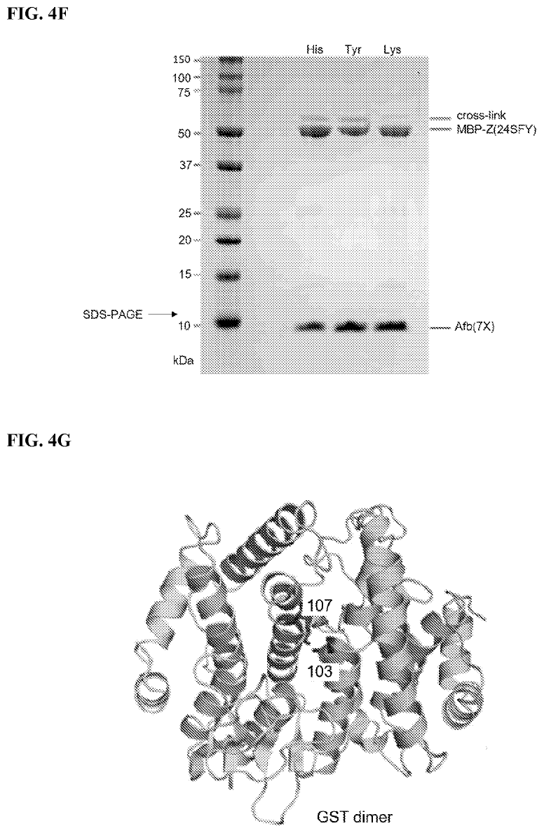

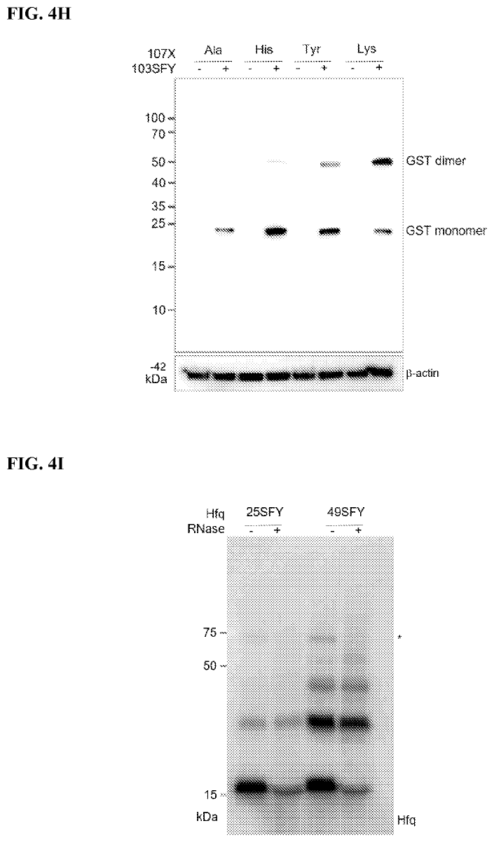

FSY has the SuFEx group at the para position, which has limited reaction area. To cope with different orientations of protein-RNA interactions, it would be desirable to encode a Uaa containing the SuFEx group at the meta position to expand reaction area. (Ref 37). We recently evolved the new orthogonal Mm-tRNA^{Pyl}/MmSFYRS and Ma-tRNA^{Pyl}/MaSFYRS to genetically incorporate o-sulfonyl fluoride-O-methyltyrosine (SFY) into proteins in E. coli (FIG. 4A). Here we demonstrate the incorporation of SFY into proteins in mammalian cells and the ability of SFY to crosslink proximal nucleophilic amino acid sidechains via SuFEx directly in E. coli and mammalian cells.

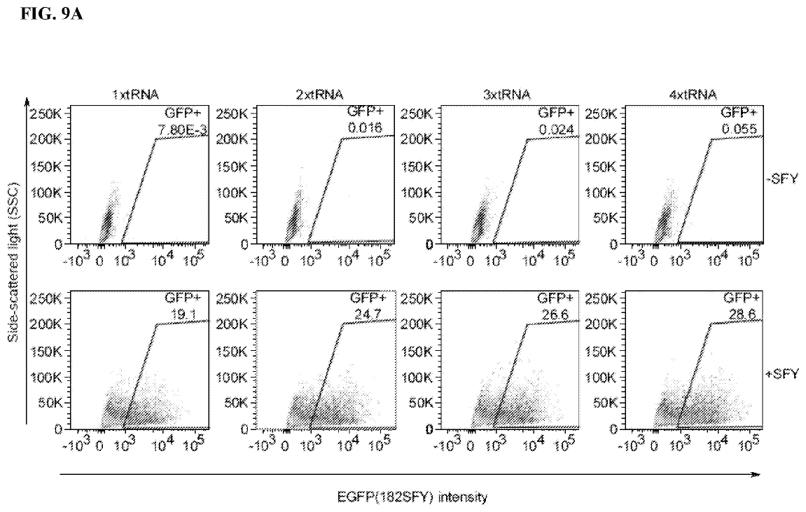

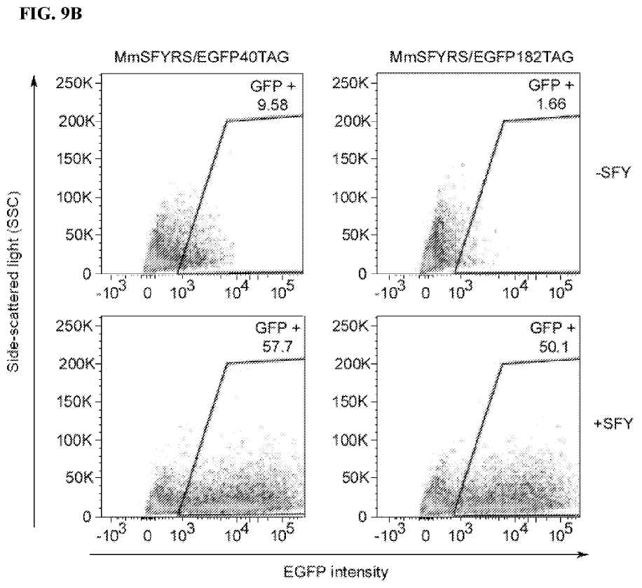

To test SFY incorporation in mammalian cells, we transfected HEK293 cells with plasmid pcDNA-EGFP-40TAG expressing EGFP gene containing a TAG codon at site Tyr40 and plasmid pNEU-MmSFYRS expressing the Mm-tRNA^{Pyl}/MmSFYRS. Fluorescence confocal microscopy showed that, in the presence of SFY, strong EGFP fluorescence was observed throughout the cells, and cell morphology remained normal (FIG. 4B), indicating SFY was incorporated at the TAG site to produce full-length EGFP. No fluorescence signal was detected when SFY was not added. HEK293 cells expressing pcDNA-EGFP-40TAG and Mm-tRNA^{Pyl}/MmSFYRS or Ma-tRNA^{Pyl}/MaSFYRS were further quantified by flow cytometry (FIG. 4C, FIG. 9). Strong EGFP fluorescence was measured from cells only when SFY was added, and the fluorescence intensity increased with tRNA^{Pyl} copy number. The incorporation efficiency of SFY was comparable with FSY. In addition, we did not observe obvious toxicity of SFY to HEK293T cells (FIG. 10), a valuable property for in cell applications.

To determine which amino acid residues could react with SFY via proximity-enabled reactivity directly in cells, we coexpressed in E. coli the Z protein and an affibody (Afb) specifically binding it. Based on the crystal structure of Afb-Z complex, we introduced SFY at site 24 of the Z protein and various natural residues at site 7 of the affibody (FIG. 4D), placing the two residues in close proximity upon Afb-Z binding. (Ref 22). After expression of Afb(24SFY) and Z(7X) (X=target residue) for 6 h, cells were lysed and analyzed with Western blot under denatured conditions (FIGS. 4E-4F). Crosslinking bands corresponding to the adduct of Afb and Z were detected for target residue His, Tyr, and Lys, for both Mm-tRNA^{Pyl}/MmSFYRS and Ma-tRNA^{Pyl}/MaSFYRS. We then purified 6×His-tagged Z and Afb proteins from cells and analyzed with SDS-PAGE. Consistently, a protein band corresponding to the cross-linked Z with Afb was clearly observed for Afb-7Lys, Afb-7His, and Afb-7Tyr (FIGS. 4E-4F). We further tested if SFY could crosslink with these residues in mammalian cells. GST is a dimeric protein, whose structure shows that residue 103 of one monomer is close to residue 107 of the other monomer at the dimer interface (FIG. 4G), which has been used to determine proximity-enabled reactivity. (Ref 38). We incorporated SFY at site 103 of GST and mutated residue 107 to various target residues. HEK293T cells expressing these GST mutants were lysed and Western blotted to detect covalent GST dimer formation (FIG. 4H). Clearly SFY was shown to react with His, Tyr, and Lys placed in proximity in mammalian cells.

We also verified if SFY incorporated into Hfq could covalently capture RNA in E. coli cells. E. coli DH10B cells expressing Hfq(25SFY) or Hfq(49SFY) were lysed and analyzed with Urea-PAGE (FIG. 4I). Crosslinking bands were detected, which disappeared when samples were treated with RNase, indicating that Hfq(SFY) was able to crosslink RNAs in E. coli.

In addition, to check if SFY could cross-link all four nucleotides, we incubated 50 mM SFY with 50 mM different nucleoside monophosphates (NMPs: AMP, UMP, CMP, or GMP) at 37° C. for 16 hours. Cross-linking adducts of SFY with all four NMPs were detected using MS, confirming SFY could also cross-link nucleotides unbiasedly (data not shown).

An in vivo method for detecting m6A in mammalian cells with single-nucleotide resolution

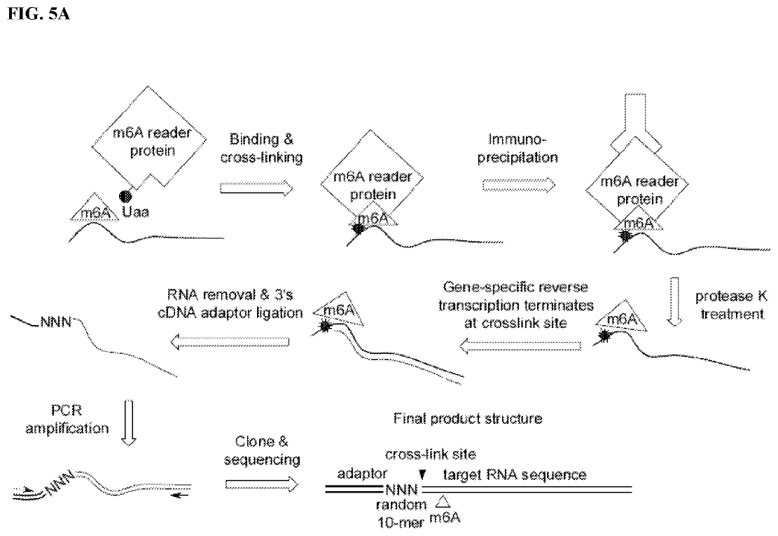

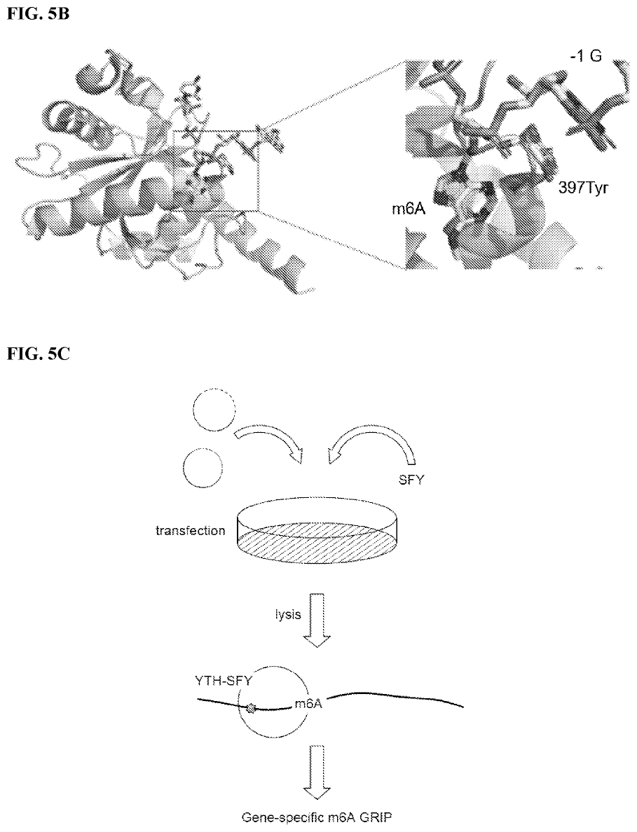

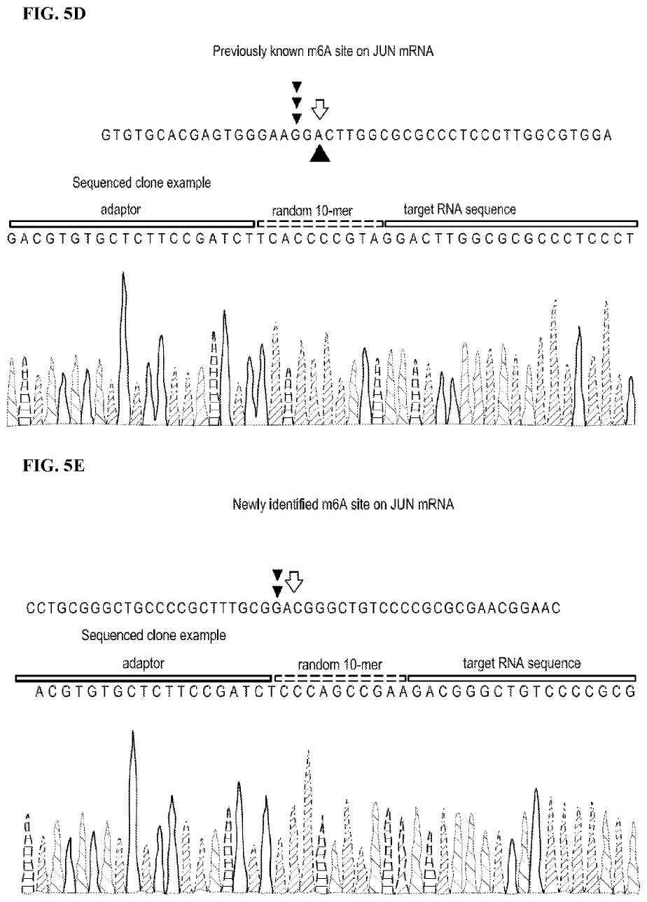

N⁶-methyladenosine (m6A) is a widespread RNA modification that play important roles in the regulations and functions of mRNA. (Ref 39). Identification of the m6A sites in mRNA is critical for understanding m6A function. Although many m6A detection methods have been reported, the majority of them lack single nucleotide resolution and rely on the use of m6A-specific antibody, in which the recognition of m6A is in vitro in nature. (Refs 40-42). Enlightened by the success of applying GRIP in identifying the crosslinked nucleotides of rpoS RNA in E. coli above, we reasoned that GRIP could be adapted for capturing m6A sites on mRNA in vivo for subsequent identification, which may better preserve m6A physiological status than the in vitro methods. Specifically, we proposed to use a reader protein of m6A to recognize m6A sites on mRNA, and to incorporate a bioreactive Uaa into the m6A binding site of the reader to cross-link nucleotides neighboring m6A (FIG. 5A). Expression of the reader-Uaa protein in cells would crosslink at m6A sites on mRNA, enabling the recognition and capture of m6A motif in vivo. Immunoprecipitation of the reader protein followed with protease K digestion then release the captured RNAs for reverse transcription, adaptor ligation, and sequencing (FIG. 5A). The identified Uaa-crosslinked nucleotides thus reveal m6A site to be immediately adjacent.

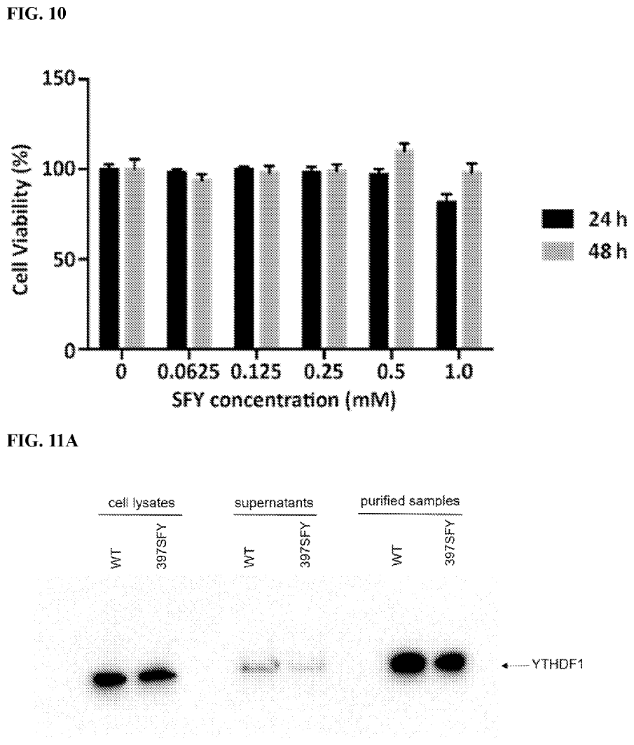

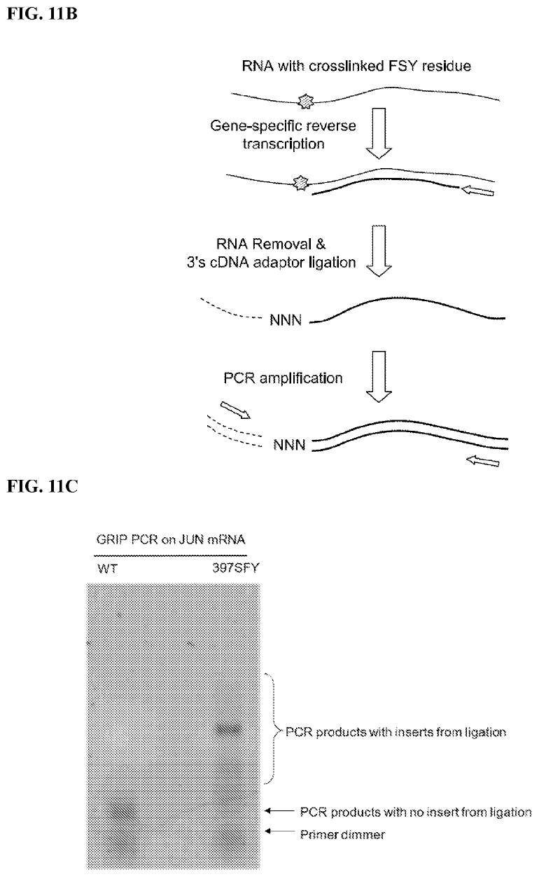

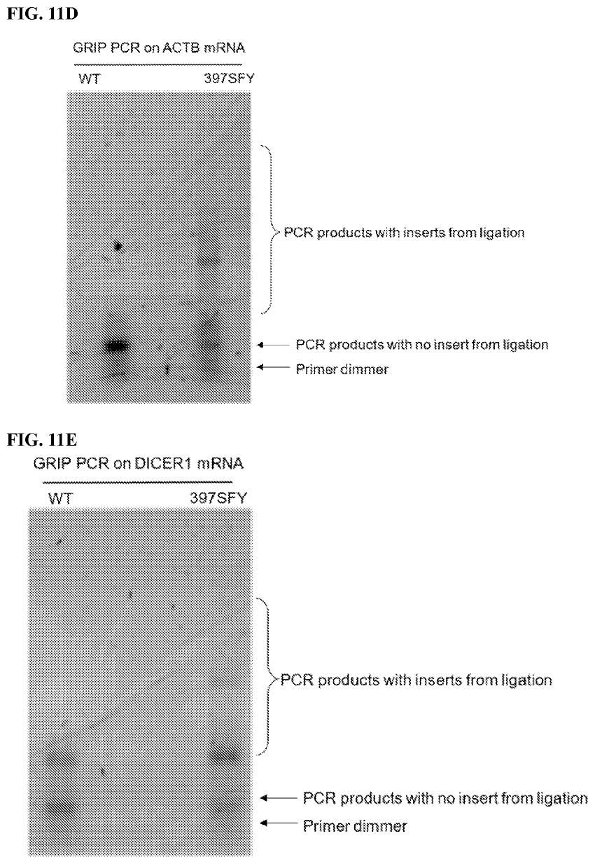

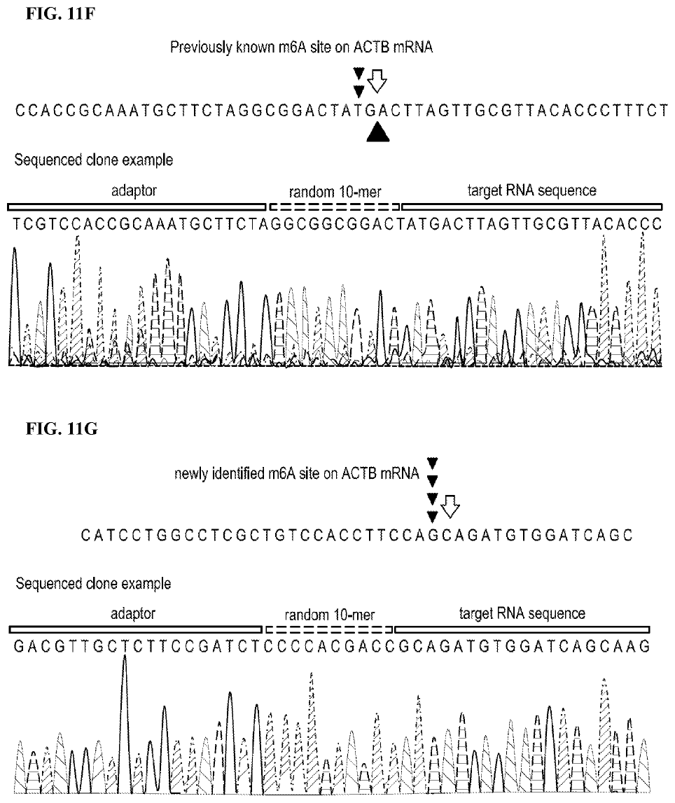

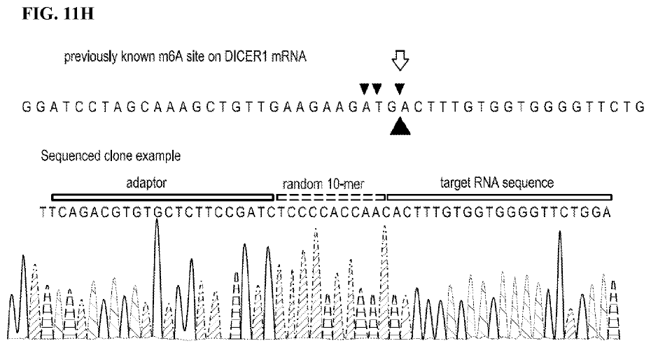

We decided to use the YTH domain of human YTHDF1 protein, which is a conserved m6A reader. (Refs 43-44). Based on the crystal structure of YTHDF1 in complex with a 5-mer m6A RNA, we chose Tyr397, a residue next to the binding pocket of m6A, as the site for incorporating the bioreactive Uaa, to aim the Uaa side chain for targeting nucleotides upstream of m6A (FIG. 5B). (Ref 43). Initial incorporation of FSY at site 397 of the YTH domain failed to crosslink any RNA. A more careful analysis of the structure revealed a Lys469 at the para-position of Tyr397, which is known to react with FSY. Therefore, we changed to the new SuFEx capable bioreactive Uaa, SFY, which has similar proximity-enabled reactivity as FSY in crosslinking nearby Lys, His, and Tyr. As SFY has sulfonyl fluoride installed at the meta-position of the phenyl ring, which should avoid Lys469 contact and reactivity (FIG. 4A, FIG. 5B). YTH-397SFY protein was expressed in HEK293T cells (FIG. 11A), followed by GRIP procedures (FIGS. 5A-5C). Three RNA regions from JUN, ACTB1, and BSG genes, containing known m6A sites, were reversely transcribed, ligated, and amplified with gene-specific primers, respectively. (Ref 45). As expected, in final PCR products YTH-WT samples had no insertion, while YTH-397SFY samples showed distinct insertions for all three genes (FIGS. 11B-11E). After cloning and sanger sequencing of YTH-397SFY PCR products, we identified crosslinking sites at nucleotides 2-3 bp upstream of previously known m6A sites for all three genes (FIG. 5E, FIGS. 11F-11H), confirming that this method was able to correctly identify m6A sites in mammalian cells as designed. Interestingly, apart from the known m6A sites, in the amplified regions of ACTB and JUN genes, we also identified two new m6A modification sites (FIG. 5E, FIGS. 11F-11H), indicating that our method was able to identify m6A sites elusive to other methods. In all identified m6A sites, the assignment of m6A was unambiguous with single-nucleotide resolution.

### DISCUSSION

Through genetic incorporation of latent bioreactive Uaas capable of reacting with RNA in proximity, we developed a novel method, genetically encoded chemical cross-linking of proteins with RNA (GECX-RNA) in vivo. GECX-RNA was able to covalently capture target RNA onto RNA-binding proteins when they interacted in vitro, in E. coli, and in mammalian cells, providing resolution of not only nucleotide but also amino acid residue. By applying GECX-RNA on RNA chaperon Hfq in E. coli, we demonstrated RNA crosslinking and identification with amino acid specificity of Hfq. Adapting GECX-RNA in mammalian cells, we developed a GRIP method for in vivo detection of m6A sites on mRNA with single-nucleotide resolution.

GECX-RNA affords several advantages over the common nucleoside-based UV crosslinking for studying protein-RNA interactions. Nucleoside-based UV crosslinking crosslinks with uridine predominantly, making it unsuitable for RNA-binding proteins that bind uridine-lacking RNAs, such as poly-A binding proteins. (Refs 9, 11, 16, 46-47). By targeting the 2′-OH group of ribose, GECX-RNA could crosslink all four nucleotides. While UV crosslinking occurs in the short UV irradiation window, GECX-RNA continuously captures RNA whenever they interact, allowing enriching the crosslinked product over a long period to improve detection sensitivity, which is particularly valuable for detecting dynamic RNA events with weak and transient interactions. (Refs 7, 20). UV light offers spatiotemporal control but cannot be applied in nontransparent animal models. In contrast, GECX-RNA reacts spontaneously upon binding, obviating the external light trigger and timing issue. Giving the ability to genetically encode Uaas in animals such as C. elegans and mouse, GECX-RNA should be compatible for in vivo use in animals for interrogation in physiological settings.

While existing methods all recognize m6A in vitro via antibody, our GECX-RNA based GRIP method represents an antibody-free approach for identifying m6A with single-nucleotide resolution in vivo, which should reflect m6A physiological status more closely. Future combination of GRIP with high-throughput sequencing will enable mapping all m6A in the transcriptome. (Ref 15). In addition, the GRIP strategy can be generalized to map other RNA modifications in vivo for which a reader or binder exists.

A key advance of GECX-RNA is gaining amino acid resolution for RNA-binding proteins, of which nucleoside-based UV crosslinking methods lack. GECX-RNA possesses dual resolutions of nucleotide for RNA and of amino acid for RNA-binding proteins, thus dramatically expanding the scope of protein-RNA studies. As a proof-of-principle, we demonstrated RNA identification of Hfq specific for site 25. Indeed, many RNA-binding proteins possess multiple RNA-binding domains, co-operations among which are critical for the RNA-binding proteins function. (Refs 48-49). A large number of novel RNA-binding regions in unconventional RBPs have recently been identified, and mutations causing Mendelian genetic diseases are found enriched in these RNA-binding regions, emphasizing the need to study these RNA-binding regions with amino acid resolution. (Refs 4, 6). However, many of these novel RNA-binding regions are intrinsically disordered, making them intractable with traditional structural biology approaches. (Ref 4). RNA-GECX will provide useful tools to investigate these emerging novel aspects of protein-RNA interactions in vivo. (Ref 19).

Lastly, we demonstrate here the selective targeting of RNA via proximity-enabled reactivity of genetically encoded latent bioreactive Uaas, which was previously limited to targeting proteins. (Ref 18). The ability to selectively engineer covalent bonds between proteins has led to a range of innovative applications, such as pinpointing ligand-receptor on live mammalian cells and developing covalent protein drugs. (Refs 19, 21). Similarly, judicious engineering of a covalent linkage between protein and RNA will inspire novel avenues for RNA-related research and therapeutics, such as live transcript imaging, epitranscriptomic modification, interrogation of lncRNA, translational modulation, RNA base editing, and so on.

### Materials and Methods

Sequences of all oligonucleotides in materials and methods are listed in Table 1.

Cloning of pBAD-dCas13 Plasmids
pBAD-PsCas13b Plasmids

PsCas13b was PCR amplified from pC0046-EF1a-PspCas13b-NES-HIV plasmid (addgene #103862) with primers of pBAD-Nde1-GA-PsCas13b-F and pBAD-Hind3-GA-3HA-R. To generate pBAD-PsCas13b plasmid, the PCR product was cloned into pBAD vector pre-digested with NdeI and HindIII using Gibson Assembly kit (New England Biolabs).

To generate pBAD-dLwCas13b plasmid, residues 133 and 1058 of PsCas13b gene in pBAD-PsCas13b were mutated into alanine codons using site-directed mutagenesis with following primers: primer pair of PspCas13b-H133A-mut-F and PspCas13b-H133-mut-R for H133A mutation; primer pair of PsCas13b-H1058A-2-mut-F and PsCas13b-H1058-2-mut-R for H1058A mutation.

pBAD-dPsCas13b-TAG-Mutants for crRNA-1 and ssRNA-1 Cross-Linking

To generate pBAD-dPsCas13b-TAG mutant plasmids for crRNA-1 and ssRNA-1 cross-linking, residues 133 and 1058 of dPsCas13b gene in pBAD-dPsCas13b were mutated into an amber stop codon TAG, respectively, using site-directed mutagenesis with following primers: primer pair of PspCas13b-H133TAG-mut-F and PspCas13b-H133-mut-R for 133TAG mutation, final plasmid: pBAD-dPsCas13b-133TAG; (2) primer pair of PsCas13b-H1058TAG-2-mut-F and PsCas13b-H1058-2-mut-R for H105TAG mutation, final plasmid pBAD-dPsCas13b-1058TAG. All pBAD-PsCas13b plasmids contain HA tag and 6His tag at C-terminals.

pBAD-dPsCas13b-Mutants for Pre-crRNA Cleavage and Cross-Linking Assay

To generate pBAD-dPsCas13b mutant plasmids for pre-crRNA cleavage and cross-linking assay, residues 367, 370, 378, and 380 of dPsCas13b gene in pBAD-dPsCas13b were mutated into either an alanine codon or an amber stop codon TAG, respectively, using site-directed mutagenesis with following primers: (1) primer pair of Ps13b-K367A-v-F and Ps13b-K367A-v-R for K367A mutation, final plasmid: pBAD-dPsCas13b-367A, (2) primer pair of Ps13b-K370A-v-F and Ps13b-K367A-v-R for K370A mutation, final plasmid: pBAD-dPsCas13b-370A, (3) primer pair of Ps13b-R378A-v-F and Ps13b-R378A-v-R for R378A mutation, final plasmid: pBAD-dPsCas13b-378A, (4) primer pair of Ps13b-R380A-v-F and Ps13b-R380A-v-R for R380A mutation, final plasmid: pBAD-dPsCas13b-380A, (5) primer pair of Ps13b-K367U-v-F and Ps13b-K367U-v-R for K367TAG mutation, final plasmid: pBAD-dPsCas13b-367TAG, (6) primer pair of Ps13b-K370U-v-F and Ps13b-K367U-v-R for K370TAG mutation, final plasmid: pBAD-dPsCas13b-370TAG, (7) primer pair of Ps13b-R378U-v-F and Ps13b-R378U-v-R for R378TAG mutation, final plasmid: pBAD-dPsCas13b-378TAG, (8) primer pair of Ps13b-R380U-v-F and Ps13b-R380U-v-R for R380TAG mutation, final plasmid: pBAD-dPsCas13b-380TAG. All pBAD-PsCas13b plasmids contain HA tag and 6His tag at C-terminals.

dCas13b Protein Expression and Purification
dPsCas13b-WT; dPsCas13b-360A; dPsCas13b-367A; dPsCas13b-370A; dPsCas13b-378A; dPsCas13b-380A

pBAD-dPsCas13b plasmid (dPsCas13b-WT, dPsCas13b-360A, dPsCas13b-367A, dPsCas13b-370A, dPsCas13b-378A, or dPsCas13b-380A) was transformed into DH10B E. coli chemical competent cells. The transformants were plated on an LB-Amp100 agar plate and incubated overnight at 37° C. A single colony was inoculated into 5 mL of 2×YT-Amp100 and cultured overnight at 37° C. On the following day, 1 mL of overnight cell culture was diluted into 50 mL 2×YT-Amp100 and agitated vigorously at 37° C. When OD600 reached 0.4˜0.6, the cell culture was induced with 0.2% arabinose, then incubated at 18° C. for 18 h. Cell pellets were collected by centrifugation at 4200 g for 30 min at 4° C. and stored at −80° C.

dPsCas13b-133FSY; dPsCas13b-1058FSY; dPsCas13b-360FSY; dPsCas13b-367FSY; dPsCas13b-370FSY; dPsCas13b-378FSY; dPsCas13b-380FSY

pBAD-dPsCas13b TAG mutant plasmid (pBAD-dPsCas13b-133TAG, pBAD-dPsCas13b-1058TAG, dPsCas13b-360TAG, dPsCas13b-367TAG, dPsCas13b-370TAG, dPsCas13b-378 TAG, or dPsCas13b-380TAG) was co-transformed with pEvol-FSYRS (encoding FSY-tRNA synthetase-tRNA system for expression in e. coli cells) into DH10B E. coli chemical competent cells, respectively. Wang et al, J. Am Chem. Soc., 140:4995-4999 (2018). The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD600 reached 0.4˜0.6, the cell culture was induced with 0.2% arabinose and 1 mM FSY (FSY was chemically synthesized as previously reported¹), then incubated at 18° C. for 18 h. Cell pellets were collected by centrifugation at 4200 g for 30 min at 4° C. and stored at −80° C.

### His-Tag Protein Purification

Above cell pellets were resuspended in 14 mL lysis buffer (20 mM Tris-HCl pH 7.4, 500 mM NaCl, 20 mM imidazole, lysozyme 1 mg/mL, and protease inhibitors). The cell suspension was lysed at 4° C. for 30 min. Cell lysate was sonicated with Sonic Dismembrator (Fisher Scientific, 30% output, 3 min, 1 sec off, 1 sec on) in an ice-water bath, followed by centrifugation (20,000 g, 30 min, 4° C.). The soluble fractions were collected and incubated with pre-equilibrated Protino® Ni-NTA Agarose resin (400 μL) at 4° C. for 1 h with constant mechanical rotation. The slurry was loaded onto a Poly-Prep® Chromatography Column, washed with 5 mL of wash buffer (20 mM Tris-HCl pH 7.4, 500 mM NaCl, 20 mM imidazole, 2 mM DTT) for 3 times, and eluted with 200 μL of elution buffer (20 mM Tris-HCl pH 7.4, 500 mM NaCl, 500 mM imidazole, 2 mM DTT, 10% glycerol) for 5 times. The eluates were concentrated, and buffer exchanged into 100 μL of protein storage buffer (20 mM Tris-HCl pH 7.4, 500 mM NaCl, 2 mM DTT, 10% glycerol) using Amicon Ultra columns, and stored at −80° C. for future analysis.

### RNA Transcription and Labeling

Templates for crRNA (crRNA-1) were PCR amplified with following primers (T7pro-Ps13b-cr-1-F and Ps13b-crRNA-1-R) to yield dsDNA and then incubated with T7 polymerase at 37° C. overnight using the MAXIscript T7 transcription kit (Thermo Fischer Scientific). crRNA-1 was purified using Clean and Concentrator columns (Zymo Research).

Templates for target RNA (ssRNA-1) were PCR amplified with following primers (primer pair of T7pro-ssRNA-1-F and ssRNA-1-R for target RNA ssRNA-1) to yield dsDNA and then incubated with T7 polymerase at 37° C. overnight using the MAXIscript T7 transcription kit. Target RNAs (ssRNAs) were purified using Clean and Concentrator columns. 5′ end labeling was performed on target RNA (ssRNA-1) using the 5′ oligonucleotide kit (VectorLabs, Burlingame, CA) and with a maleimide-IR800 probe (LI-COR Biosciences, Lincoln, NE). Labeled target RNAs (5′IRD680-ssRNA-1) were purified using Clean and Concentrator columns.

Templates for pre-crRNAs were PCR amplified using PCR amplicon of crRNA-1 as template with following primers (primer pair of T7pro-ssRNA-1-F and Ps13b-pre-crRNA-AAA-R for pre-crRNA-AAA; primer pair of T7pro-ssRNA-1-F and Ps13b-pre-crRNA-UUU-R for pre-crRNA-UUU; primer pair of T7pro-ssRNA-1-F and Ps13b-pre-crRNA-CCC-R for pre-crRNA-CCC; primer pair of T7pro-ssRNA-1-F and Ps13b-pre-crRNA-GGG-R for pre-crRNA-GGG) to yield dsDNA and then incubated with T7 polymerase at 37° C. overnight using the MAXIscript T7 transcription kit (Thermo Fischer Scientific). pre-crRNAs were purified using Clean and Concentrator columns (Zymo Research).

dCas13b-crRNA-ssRNA In Vitro Cross-Linking Assay

dCas13b cross-linking assays were performed with 10 nM of unlabeled or 5′-IRD-680 labeled ssRNA target, 100 nM purified dPsCas13b proteins, and 30 nM crRNA in 40 mM TrisHCl, 60 mM NaCl, 10 mM EDTA, 10 μg/mL heparin, pH 7.4. Incubations were performed at 37° C. overnight. After incubation, the samples were then denatured with laemmli sample buffer (1% LDS, 50 mM DTT) at 95° C. for 5 minutes. Samples were analyzed by denaturing gel electrophoresis on 10% 8 M Urea TBE PAGE. Gels were imaged by scanning fluorescent signal after SybrGold staining (for ssRNA-1), or by direct scanning with an Odyssey scanner (LI-COR Biosciences) (for 5′-IRD680-ssRNA-1).

### Pre-crRNA Cleavage Assay

pre-crRNA cleavage assays were performed with 0.2 μM of pre-crRNA-AAA and 1 μM of purified dPsCas13b proteins in 40 mM TrisHCl, 60 mM NaCl, 10 mM EDTA, 5 mM MgCl₂, pH 7.4. Incubations were performed at 37° C. for 45 min. After incubation, to quenched the reactions, the samples were immediately denatured with laemmli sample buffer (1% LDS, 50 mM DTT) at 95° C. for 5 min. Samples were analyzed by denaturing gel electrophoresis on 10% 8 M Urea TBE PAGE. Gels were imaged by scanning fluorescent signal after SybrGold staining.

dCas13b-380 Mutant Proteins and Pre-crRNA In Vitro Cross-Linking Assay

pre-crRNA cross-linking assays were performed with 40 nM of different pre-crRNAs (pre-crRNA-AAA, pre-crRNA-UUU, pre-crRNA-CCC, or pre-crRNA-GGG) and 200 nM of different purified dPsCas13b-380 mutant proteins (380A or 380FSY) in 40 mM TrisHCl, 60 mM NaCl, 10 mM EDTA, pH 7.4. Incubations were performed at 37° C. overnight. After incubation, the samples were denatured with laemmli sample buffer (1% LDS, 50 mM DTT) at 95° C. for 5 min. Samples were analyzed by denaturing gel electrophoresis on 10% 8 M Urea TBE PAGE. Gels were imaged by scanning fluorescent signal after SybrGold staining.

Cloning of pBAD-Hfq Plasmids

To generate pBAD-Hfq-WT plasmid, the Hfq encoding gene was amplified by colony PCR with primer pair of Hfq-Nde1-F and Hfq-6H-Hind3-R, digested with Nde I and Hind III, and ligated into the pBAD vector pre-treated with the same restriction enzymes.

To generate pBAD-Hfq-TAG mutant plasmids, residue 25, 30 and 49 of Hfq gene in pBAD-Hfq-WT were mutated into an amber stop codon TAG, respectively, using site-directed mutagenesis with following primers: (1) primer pair of Hfq-Tyr25TAG-F and Hfq-Tyr25TAG-R for 25TAG mutation, final plasmid: pBAD-Hfq-25TAG; (2) primer pair of Hfq-Ile30TAG-F and Hfq-Ile30TAG-R for 30TAG mutation, final plasmid: pBAD-Hfq-30TAG; (3) primer pair of Hfq-Thr49TAG-F and Hfq-Thr49TAG-R for 49TAG mutation, final plasmid: pBAD-Hfq-49TAG. All pBAD-Hfq plasmids contain 6His tag at C-terminals.

Expression of exogenous Hfq proteins in E. coli cells (Hfq-WT and Hfq-FSY samples) pBAD-Hfq-WT was transformed into DH10B E. coli chemical competent cells. The transformants were plated on an LB-Amp100 agar plate and incubated overnight at 37° C. A single colony was inoculated into 5 mL of LB-Amp100 and cultured overnight at 37° C. On the following day, 1 mL of overnight cell culture was diluted into 15 mL LB-Amp100 and agitated vigorously at 37° C. When OD600 reached 0.4˜0.6, the cell culture was induced with 0.2% arabinose, then incubated at 37° C. for 16 h. Cell pellets were collected by centrifugation at 4200 g for 30 min at 4° C. and stored at −80° C.

pBAD-Hfq TAG mutant plasmids (pBAD-Hfq-25TAG, pBAD-Hfq-30TAG, pBAD-Hfq-49TAG) was co-transformed with pEvol-FSYRS¹ into DH10B E. coli chemical competent cells, respectively. The transformants were plated on an LB-Amp100Cm34 agar plate and incubated overnight at 37° C. A single colony was inoculated into 5 mL of LB-Amp100Cm34 and cultured overnight at 37° C. On the following day, 1 mL of overnight cell culture was diluted into 15 mL LB-Amp100Cm34 and agitated vigorously at 37° C. When OD600 reached 0.4˜0.6, the cell culture was induced with 0.2% arabinose and 1 mM FSY, then incubated at 37° C. for 16 h. Cell pellets were collected by centrifugation at 4200 g for 30 min at 4° C. and stored at −80° C.

RNase Treatment and Detection for Exogenous Hfq-Expressing E. coli Cells (Hfq-WT and Hfq-FSY Samples)

For exogenous Hfq-expressing E. coli cell pellets (Hfq-WT and Hfq-FSY samples), 100 μL PBS were added to resuspended the cell pellets. Then 200 μL 0.5 mm glass beads were added. The samples were then put into dry ice, and then lyophilized for 3 hr. Dried cells were disrupted by vortexing for 5 min, at intervals of 1 min to avoid warming the sample. The disrupted samples were then resuspended in 1 ml PBS. For RNase treated samples, 10 μL of resuspended disrupted sample was aliquoted, added with 1U/μL of RNase A (Qiagen) and protease inhibitors, shaking-incubated @ 37° C. for 1 hr, then boiled with laemmli buffer and loaded on SDS-PAGE. For samples without RNase treatment, 10 μL of resuspended disrupted sample was directly boiled with laemmli buffer and loaded on SDS-PAGE. The SDS-PAGE was then separated via electrophoresis and immunoblotted with 1:10000 anti-his monoclonal antibody (Proteintech #HRP-66005) to detect exogenously expressed Hfq proteins.

### Purification and Quantification for Hfq-RNA Binding

For exogenous Hfq-expressing E. coli cell pellets, 100 μL PBS were added to resuspended the cell pellets. Then 200 μL 0.5 mm glass beads were added. The samples were then put into dry ice, and then lyophilized for 3 hr. Dried cells were disrupted by vortexing for 5 min, at intervals of 1 min to avoid warming the sample. The disrupted samples were then resuspended in 1.4 ml of 6M GuHCl, 400 mM NaCl, 1×PBS, 10 mM imidazole, 0.2% TritonX100, 0.5 mM DTT, and centrifuged at 10° C., 15000g for 10 min. Two sets of 10 μL of the supernatants were preserved as cell lysate samples for RNA extraction and western blot detection. The supernatant was added with 20 μL of HisPur magnetic beads (thermo fisher) and rotated at room temperature for 1.5 hr. Then the beads were washed twice with 6 M GuHCl, 400 mM NaCl, 1×PBS, 10 mM imidazole, 0.2% TritonX100, 0.5 mM DTT, washed once with 1×150 mM NaCl, PBS at 4° C., washed once with 1×PBS at 4° C., shaking-incubated with 50 μL TurboDNase (2U) at 37° C. for 15 min, washed once with ddH₂O at room temperature. 1/10 of the beads was then aliquoted as purified samples preserved for western blot detection. The rest of beads was then shaking-incubated with 50 μL of 5 mg/ml protease K, 2M urea at 37° C. for 1 h.

Preserved cell lysate samples and beads for western blot detection were boiled with laemmli buffer, separated on SDS-PAGE, and immunoblotted with 1:10000 anti-his monoclonal antibody (Proteintech #HRP-66005) to detect Hfq proteins.

RNA from protease-treated beads (purified samples) and preserved cell lysate samples were purified using QuickRNA micro prep kits (Zymo Research). Purified RNA was reverse transcribed to cDNA using SuperScript IV First-Strand Synthesis System (Thermofisher). Enrichments of target RNA was quantified with quantitative PCR (qPCR) using ChamQ Universal SYBR qPCR Master Mix (Vazyme). All qPCR reactions were performed in 10-μl reactions with 3 technical replicates in 96-well format and read out using a LightCycler 96 Instrument (Roche). Enrichment was quantified for samples compared with their matched control samples without Hfq exogenously expression. qPCR primers are listed in Table 1.

### GRIP for Detecting In Vivo RNA Cross-Linking Sites of FSY-Incorporated Hfq Proteins

GECX-RNA with immunoprecipitation (GRIP) was performed on RNA from purified Hfq-WT and Hfq-25FSY samples (Materials and Methods, section “Purification and quantification for Hfq-RNA binding. In general, RNA from purified Hfq samples was reverse-transcribed with gene-specific RT primers targeting different cross-linking genes and regions (rpoS-XL-RT and ptsG-xL-RT, listed in Table 1) with SuperScript IV First-Strand Synthesis System (Thermofisher). The cDNA was treated with ExoSAP-IT to remove free primers, and then treated with NaOH to degrade RNA molecules. After clean-up with DynaBeads MyONE Silane (Thermofisher), a 5′ linker (Rand3Tr3 adapter, Table 1) was ligated to cDNA molecules by T4 RNA ligase in on-beads solution with high concentration of PEG8000 at room temperature for 16 hr. The ligated product was cleaned up again with DynaBeads MyONE Silane, and then amplified with primers targeting gene-specific regions and the 5′ linker (PCR primer pair for the eCLIP region from rpoS RNA are primers pBADf-rpoS-xL-pF and pBADr-eCLIP-Rand103tr3-pR; PCR primer pair for the eCLIP region from ptsG RNA are primers pBADf-ptsG-xL-pF and pBADr-eCLIP-Rand103tr3-pR). The PCR product was separated on agarose gel. The insertion bands were cut out, purified and cloned into pBAD vector, transformed into DH10B competent cells, and plated onto LB-Amp100 agar plate and incubated overnight at 37° C. Plasmids were then extracted from colonies and sequenced. The sequenced inserts from plasmids were aligned to target RNA (rpoS RNA or ptsG RNA). The ligation sites of 5′ linker represent the cross-linking sites of Hfq-25FSY proteins on target RNA molecules.

Cloning of pcDNA3.1-dPsCas13b Plasmids

To generate plasmids suitable for expressing dPsCas13b proteins in mammalian cells, dPsCas13b-WT insert and dPsCas13b-H133TAG insert were amplified from pBAD-dPsCas13b and pBAD-dPsCas13b-H133TAG using primer pair of pcDNA31-Hind3-PsCas13b-F and pcDNA31-BamH1-PsCas13b-R, and cloned into pcDNA3.1 vector pre-digested with BamHI and HindIII using Gibson Assembly kit (New England Biolabs).

Cloning of pC0046-crRNA Plasmids

To generate plasmids suitable for expressing crRNA of dPsCas13b in mammalian cells, different crRNA inserts were PCR amplified with different pairs of primers (primers for crACTB are pc43-Ps13b-crACTB1-F and pc43-Ps13b-cr-R; primers for crNEATT-1 are pc43-Ps13b-NEAT1-1-F and pc43-Ps13b-cr-R; primers for crNEAT1-2 are pc43-Ps13b-crNEAT1-2-F and pc43-Ps13b-cr-R), and cloned into pC0043-PspCas13b crRNA backbone (addgene #103854) vector pre-digested with KpnI and BbsI using ClonExpress II one step cloning kit (Vazyme).

Purification and Quantification for dPsCas13b-RNA Binding in Mammalian Cells with RNA Immunoprecipitation

For RNA immunoprecipitation experiments, HEK293T cells were plated in six-well plates and transfected with 1 μg of pcDNA3.1-dPsCas13b plasmids and 1 μg of pC0043-PspCas13b crRNA plasmids, with an additional 1 μg of pMP-FSYRS plasmid¹ (encoding FSY-tRNA synthetase-tRNA system for expression in mammalian cells) and 1 mM FSY for conditions involving Cas13b-133FSY protein expression. Forty-eight hours after transfection, cells were washed twice with ice-cold PBS, and centrifuged to collect as cell pellets. Cells were lysed with 200 μL of 1×RIPA Buffer (Thermofisher) supplemented with protease inhibitors and RNase inhibitor. Cells were lyzed on ice for 10 min and then passed through 26G-needles for 10 times to achieve full lysis. Lysates were then pelleted by centrifugation at 16,000 g for 10 min at 4° C., and the supernatants containing cleared lysates were used for pulldown with magnetic beads.

To conjugate antibodies to magnetic beads, 100 μL per sample of Dynabeads Protein G for Immunoprecipitation (Thermo Fisher Scientific) were pelleted by application of a magnet, and the supernatant was removed. Beads were resuspended in 200 μL of wash buffer (PBS, 0.02% Tween 20) and 5 pg of anti-HA antibody (Thermo Fisher Scientific 26183) was added. The sample was incubated for 10 min at room temperature on a rotator for antibody-beads conjugation. After incubation, beads were pelleted using a magnet, supernatant was removed, and beads were washed twice with wash buffer, and resuspended in 200 μL 1×RIPA with protease inhibitors and RNase inhibitor. 200 μL of sample lysate were added to beads and rotated overnight at 4° C.

After incubation with sample lysate, beads were pelleted, washed four times with 1×RIPA, 0.02% Tween 20, and then washed with DNase buffer (350 mM Tris-HCl (pH 6.5); 50 mM MgCl2; 5 mM DTT). Beads were resuspended in DNase buffer and TURBO DNase was added to a final concentration of 0.1 U/μL. DNase was shaking-incubated for 30 min at 37° C. Proteins were then digested by shaking-incubation with 50 μL of 5 mg/ml protease K, 2M urea at 37° C. for 1 hr.

RNA was purified using QuickRNA micro prep kits. Purified RNA was reverse transcribed to cDNA using SuperScript IV First-Strand Synthesis System. Enrichments of target RNA was quantified with qPCR using ChamQ Universal SYBR qPCR Master Mix. All qPCR reactions were performed in 10-μL reactions with 3 technical replicates in 96-well format and read out using a LightCycler 96 Instrument. Enrichment was quantified for samples compared with their matched control cells without crRNA transfection. qPCR primers used are listed in Table 1.

Cloning of pNEU-MmSFYRS-4xU6M15 Plasmid

The MmSFYRS gene was amplified with primers HR-MmPyIRS-NheI-F/HR-MmPyIRS-NotI-R and ligated into pNEU-XYRS-4xU6M15 (derived from pNEU-hMbPyIRS-4xU6M15, a gift from Irene Coin, Addgene plasmid #105830) which was linearized with NheI/NotI to generate pNEU-MmSFYRS-4xU6-M15.

Cloning of pNEU-MaSFYRS-NxU6-MaPylT (N=1 to 4) Plasmids

The MaSFYRS and Ma-PylT expression cassettes were cloned into pNEU-XYRS-4xU6M15. Specifically, the U6 promoter was amplified from pNEU-XYRS-4xU6M15 with primers U6-F1/U6-R1, and the evolved Ma-PylT(6) was amplified from pEvol-MaSFYRS with primers Ma-PylT(6)-F2/Ma-PylT(6)-R2. The resulting fragments were joined together by overlapping PCR with primers U6-F1/Ma-PylT(6)-R2 and then amplified again with primers HR-pNEU-tRNA-XhoI-F/HR-pNEU-tRNA-SalI-R to generate a monomeric U6-MaPylT expression cassette containing XbaI-XhoI and SalI restriction sites. The first monomeric U6-MaPylT expression cassette was ligated into pNEU-XYRS-4xU6M15 vector which was linearized withXhoI/SalI to generate pNEU-XYRS-1xU6-MaPylT. Then the MaSFYRS was amplified from pEvol-MaSFYRS with primers HR-Ma-SFYRS-NheI-F/HR-Ma-SFYRS-NotI-R and ligated into pNEU-XYRS-1xU6-MaPylT vector which was linearized with NheI/NotI to generate pNEU-MaSFYRS-1xU6-MaPylT. The second U6-MaPylT cassette was digested with XbaI/SalI and ligated into pNEU-MaSFYRS-1xU6-MaPylT vector that was linearized with XbaI/XhoI to generate pNEU-MaSFYRS-2xU6-MaPylT. Two more U6-MaPylT cassettes were tandemly introduced into the pNEU-MaSFYRS vector following the same procedure to construct the pNEU-MaSFYRS-4xU6-MaPylT.

Cross-Linking of MBP-Z24SFY and Afb_{4A}-7X in Live E. coli Cells

The pET-Duet-Afb_{4A}-7X-MBP-Z24TAG (X=A, C, S, T, H, Y, or K)¹ was co-transformed with pEvol-MmSFYRS and pEvol-MaSFYRS² respectively into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.5 mM IPTG and 0.2% arabinose in the presence of 1 mM SFY, and then incubated at 37° C. for 6 h. 1 mL of cell pellets were collected by centrifugation at 21000 g for 5 min at 4° C. and directly applied for immunoblot analysis. The rest of cell pellets were collected by centrifugation at 4200g for 30 min at 4° C. The cross-linking products of MBP-Z24SFY and Afb_{4A}-7X (X=H, Y, or K) with affinity chromatography as described previously¹.

### Cross-Linking of GST-103SFY-107X in Live Mammalian Cells

One day before transfection, 3×105 HEK293T cells were seeded in a Greiner 6-well cell culture dish containing 2 mL of DMEM media with 10% FBS, and incubated at 37° C. in a CO₂ incubator. 1 pg of pcDNA-GST-103TAG-107X (X=A, H, Y or K)³ and 1 pg of pNEU-MmSFYRS-4xU6M15 were co-transfected into target cells using 5 μL of lipofectamine 2000 following the manufacturer's instructions. Six hours post transfection, the media were replaced with complete DMEM media with or without 1 mM SFY. The cells were incubated at 37° C. for additional 48 h, collected, and applied for immunoblot analysis.

### Fluorescence Confocal Microscopy

One day before transfection, 3×10⁵ HEK293T cells were seeded in a Greiner 6-well cell culture dish containing 2 mL of DMEM media with 10% FBS, and incubated at 37° C. in a CO₂ incubator. Plasmids pcDNA-EGFP-40TAG (1 μg) and pNEU-MmSFYRS-4xU6M15 (1 μg) were co-transfected into target cells using 5 μL of lipofectamine 2000 following the manufacturer's instructions. Six hours post transfection, the media were replaced with complete DMEM media with or without 1 mM SFY. The cells were incubated at 37° C. for additional 24-48 h and imaged with Nikon Eclipse Ti confocal microscope.

### FACS Analysis of SFY Incorporation

One day before transfection, 3×105 HEK293T cells were seeded in a Greiner 6 well-cell culture dish containing 2 mL of DMEM media with 10% FBS, and incubated at 37° C. in a CO₂ incubator. Plasmids pcDNA-EGFP-40TAG (1 μg) and pNEU-MaSFYRS-NxU6-MaPylT (N=1 to 4) (1 μg) were co-transfected into target cells using 5 μL of lipofectamine 2000 following the manufacturer's instructions. Six hours post transfection, the media containing transfection complex were replaced with fresh DMEM media with 10% FBS in the presence or absence of 1 mM SFY. After incubation at 37° C. for 24-48 h, transfected cells were trypsinized and collected by centrifugation (1500 rpm, 5 min, r.t.). The cells were resuspended in 500 μL of FACS buffer (1×PBS, 2% FBS, 1 mM EDTA, 0.1% sodium azide, 0.28 M DAPI) and analyzed by BD LSRFortessa™ cell analyzer.

### Cell Viability Assay

2×10⁴ cells/well of HEK293T cells were seeded in a 96-well plate. On the next day, the media were replaced with fresh DMEM media supplemented with 0, 0.0625, 0.125, 0.25, 0.5, or 1 mM of SFY. The SFY-treated and control cells were cultured for an additional 24-48 h at 37° C. and then analyzed with CellTiter-Blue® Cell Viability Assay following the manufacturer's instructions.

RNase Treatment and Detection for Exogenous Hfq-Expressing E. coli Cells (Hfq-SFY Samples)

The procedure is the same as the RNase treatment and detection for exogenous Hfq-expressing E. coli cells(Hfq-WT and Hfq-FSY samples), with the following modifications:

For the transformations, pBAD-Hfq TAG mutant plasmids (pBAD-Hfq-25TAG, pBAD-Hfq-49TAG) was co-transformed with pEvol-MmSFYRS into DH10B E. coli chemical competent cells, respectively.

For the exogenous expression of Hfq-SFY proteins, the cell culture was induced with 0.2% arabinose and 1 mM SFY.

### In Vitro Incubations of NMPs and SFY

50 mM SFY (HCl salt) and 50 mM NMP was incubated in DI H2O. 50 mM NaOH was added to neutralize the HCl salt. The mixture was incubated at 37° C. for 48 h. Then the reaction mixture was diluted for 50 times in H₂O/acetonitrile (50/50, v/v, with 0.1% trifluoracetic acid) and subjected to mass spectrum analysis using positive mode. Mass spectrum analysis was performed on SCIEX MDS, 3200 Q TRAP system.

The molecular weight (MW) of addict products between SFY and NMP was calculated following this equation: MW (adduct product)=MW (SFY)+MW (NMP)−MW (HF).

| | | | Expected | Expected | Observed |
|---|---|---|---|---|---|
| | | | MW | MW | MW |
| | MW of | MW of | of adduct | of adduct | of adduct |
| | NMP | SFY | products | products + H⁺ | products + H⁺ |
| AMP | 347.0631 | 277.0420 | 604.0989 | 605.1062 | 605.0 |
| GMP | 363.0580 | | 620.0938 | 621.1011 | 620.8 |
| UMP | 324.0359 | | 581.0717 | 582.0789 | 581.7 |
| CMP | 323.0519 | | 580.0876 | 581.0949 | 580.9 |

Cloning of YTH Domain from Human YTHDF1 Protein

To generate plasmids expressing YTH domain from human YTHDF1 protein with TwinStrep tag and HA tag at C-terminal in mammalian cells, three PCR products were prepared. Insert with YTHDF1 domain was amplified with primer pair of pc31-Hd3-YTHDF1-F and YTHDF1-2xstrep-R using cDNA reverse-transcribed from total RNA of HEK293T cells as template. Insert with TwinStrep tag was amplified with primer pair of 2xstrep-tag_Hs-F and 2xstrep-tag_Hs-R. pcDNA3.1 vector backbone was amplified with primer pair of pc31-HA-strep-F and pc31-Nde1-R using empty pcDNA3.1 vector as template. The final plasmid pcDNA3.1-HsYTHDF1-WT expressing wildtype YTHDF1 domain with TwinStrep tag and HA tag at C-terminal was cloned by ligating these three PCR products together using ClonExpress II one step cloning kit (Vazyme).

To generate pcDNA3.1-HsYTHDF1-397TAG mutant plasmid, residue 397 of YTHDF1 gene in pcDNA3.1-HsYTHDF1-WT were mutated into an amber stop codon TAG using site-directed mutagenesis with following primers: YTHDF1-Y397TAG-F and YTHDF1-Y397TAG-R.

### GRIP for In Vivo m6A Detection

HEK293T cells were plated in 15-cm plates and transfected with 15 μg of pcDNA3.1-HsYTHDF1 plasmids, with an additional 15 μg of pNEU-SFYRS plasmid (encoding SFY-tRNA synthetase-tRNA system for expression in mammalian cells) and 1 mM SFY for conditions involving YTHDF1-397SFY protein expression. Forty-eight hours after transfection, cells were washed twice with ice-cold PBS, and centrifuged to collect as cell pellets. Cells were lysed with 1.5 mL of 1×RIPA Buffer supplemented with protease inhibitors and RNase inhibitor. Cells were lysed on ice for 10 min and then passed through 26G-needles for 20 times to achieve full lysis. Lysates were then pelleted by centrifugation at 16,000 g for 10 min at 4° C., and the supernatants containing cleared lysates were used for pulldown with magnetic beads.

For strep-tactin-XT magnetic beads (Iba-lifesciences), 200 μL per sample of beads were pelleted by application of a magnet, and the supernatant was removed. Beads were washed twice with wash buffer (PBS buffer with 6 M Urea, 1 M NaCl, 1 mM DTT), and resuspended in 11.25 mL of wash buffer (PBS buffer with 6M Urea, 1 M NaCl, 1 mM DTT). 750 μL of sample lysate were added to beads and rotated overnight at 4° C.

After incubation with sample lysate, beads were pelleted, washed three times with 6M Urea, 1 M NaCl, PBS buffer, 1 mM DTT, wash once with PBS buffer with 1 M NaCl, wash once with PBS buffer, and then washed with DNase buffer (350 mM Tris-HCl (pH 6.5); 50 mM MgCl2; 5 mM DTT). Beads were resuspended in DNase buffer and TURBO DNase was added to a final concentration of 0.1 U/μL. DNase was shaking-incubated for 30 min at 37° C. Proteins were then digested by shaking-incubation with 50 μL of 5 mg/mL protease K, 2 M urea at 37° C. for 1 h. RNA was purified using QuickRNA micro prep kits.

RNA samples were reverse-transcribed with gene-specific RT primers targeting different cross-linking genes and regions (ACTB-m6A-1-RT, DICER1-m6A-1-RT, and JUN-m6A-1-RT, as listed in Table 1) with SuperScript IV First-Strand Synthesis System. The cDNA was treated with ExoSAP-IT to remove free primers, and then treated with NaOH to degrade RNA molecules. After clean-up with DynaBeads MyONE Silane, a 5′ linker (Rand3Tr3 adapter, Table 1) was ligated to cDNA molecules by T4 RNA ligase in on-beads solution with high concentration of PEG8000 at room temperature for 16 h. The ligated product was cleaned up again with DynaBeads MyONE Silane, and then amplified with primers targeting gene-specific regions and the 5′ linker (PCR primer pair for the GRIP region of ACTB RNA are primers pBADf-ACTB-m6A-1-pF and pBADr-eCLIP-Rand103tr3-pR; PCR primer pair for the GRIP region of DICER1 RNA are primers pBADf-DICER1-m6A-1-pF and pBADr-eCLIP-Rand103tr3-pR; and PCR primer pair for the GRIP region of JUN RNA are primers pBADf-JUN-m6A-1-pF and pBADr-eCLIP-Rand103tr3-pR). The PCR product was separated on agarose gel. The insertion bands were cut out, purified and cloned into pBAD vector, transformed into DH10B competent cells, and plated onto LB-Amp100 agar plate and incubated overnight at 37° C. Plasmids were then extracted from colonies and sequenced. The sequenced inserts from plasmids were aligned to target RNA regions (ACTB, DICER1, or JUN), the ligation sites of 5′ linker represent the cross-linking sites of YTHDF1-397SFY proteins on target RNA molecules, thus also representing m6A sites on target RNA molecules.

TABLE 1

| Oligonucleotide name | Sequence (5′ - 3′) |
|---|---|
| pBAD-Ndel-GA-PsCas13b-F | AAGAAGGAGATATACATATG |
| | AACATCCCCGCTCTGGTGGA |
| | (SEQ ID NO: 269) |
| pBAD-Hind3-GA-3HA-R | CATCCGCCAAAACAGCCAAG |
| | CTTTTAGTGATGGTGATGGT |
| | GATGGGCATAGTCGGGGACA |
| | (SEQ ID NO: 270) |
| PspCas13b-H133-mut-R | TCCCTGTACATCTTCAGC |
| | (SEQ ID NO: 271) |
| PspCas13b-H133A-mut-F | CCTGACCAACGCCTACAAGA |
| | CCTAC |
| | (SEQ ID NO: 272) |
| PsCas13b-H1058-2-mut-R | CGGATCTTCCGCAGGATGTC |
| | GC |
| | (SEQ ID NO: 273) |
| PsCas13b-H1058A-2-mut-F | GAACGCCTTCGATGCCAACA |
| | ATTACCCC |
| | (SEQ ID NO: 274) |
| PspCas13b-H133TAG-mut-F | CCTGACCAACTAGTACAAGA |
| | CCTAC |
| | (SEQ ID NO: 275) |
| PsCas13b-H1058TAG- | GAACGCCTTCGATTAGAACA |
| 2-mut-F | ATTACCCC |
| | (SEQ ID NO: |
| | 276) |
| Ps13b-K367A-v-F | TGAGATACCTGCTGgccGCC |
| | GACAAGACCTGCATC |
| | (SEQ ID NO: 277) |
| Ps13b-K367A-v-R | ggcCAGCAGGTATCTCAGCT |
| | TGCCCATGTTCA |
| | (SEQ ID NO: 278) |
| Ps13b-K367U-v-F | AGATACCTGCTGtagGCCGA |
| | CAAGACCTGCATCG |
| | (SEQ ID NO: 279) |
| Ps13b-K367U-v-R | GCctaCAGCAGGTATCTCAG |
| | CTTGCCCATGTT |
| | (SEQ ID NO: 280) |
| Ps13b-K370A-v-F | GACgccACCTGCATCGACGG |
| | CCAGACCAGAGT |
| | (SEQ ID NO: 281) |
| Ps13b-K370A-v-R | TCGATGCAGGTggcGTCGGC |
| | CTTCAGCAGGTATCT |
| | (SEQ ID NO: 282) |
| Ps13b-K370U-v-F | GACtagACCTGCATCGACGG |
| | CCAGACCAGAGT |
| | (SEQ ID NO: 283) |
| Ps13b-K370U-v-R | TCGATGCAGGTctaGTCGGC |
| | CTTCAGCAGGTATCT |
| | (SEQ ID NO: 284) |
| Ps13b-R378A-v-F | ACCgccGTCAGAGTGATCGA |
| | GCAGCCCCTGAA |
| | (SEQ ID NO: 285) |
| Ps13b-R378A-v-R | ATCACTCTGACggcGGTCTG |
| | GCCGTCGATGCA |
| | (SEQ ID NO: 286) |
| Ps13b-R378U-v-F | CAGACCtagGTCAGAGTGAT |
| | CGAGCAGCCCCT |
| | (SEQ ID NO: 287) |
| Ps13b-R378U-v-R | ACTCTGACctaGGTCTGGCC |
| | GTCGATGCAGGT |
| | (SEQ ID NO: 288) |
| Ps13b-R380A-v-F | AGAGTCgccGTGATCGAGCA |
| | GCCCCTGAACGG |
| | (SEQ ID NO: 289) |
| Ps13b-R380A-v-R | TCGATCACggcGACTCTGGT |
| | CTGGCCGTCGAT |
| | (SEQ ID NO: 290) |
| Ps13b-R380U-v-F | CAGAGTCtagGTGATCGAGC |
| | AGCCCCTGAACG |
| | (SEQ ID NO: 291) |
| Ps13b-R380U-v-R | CGATCACctaGACTCTGGTC |
| | TGGCCGTCGATG |
| | (SEQ ID NO: 292) |
| T7pro-Ps13b-cr-1-F | AATAATACGACTCACTATAT |
| | AGATTGCTGTTCTACCAAGT |
| | AATCCATGTTGTGGAAGG |
| | (SEQ ID NO: 293) |
| Ps13b-crRNA-1-R | TGTTGTAATAGCCCCCAAAA |
| | CTGGACCTTCCACAACATGG |
| | ATTACTTGGT |
| | (SEQ ID NO: 294) |
| T7pro-ssRNA-1-F | TAATAATACGACTCACTATA |
| | GGGGGCCAGTGAATTCGAGC |
| | TCGGTACCCGGGGATCCTCT |
| | (SEQ ID NO: 295) |
| ssRNA-1-R | GTCGAGTAGATTGCTGTTCT |
| | ACCAAGTAATCCATATTTCT |
| | AGAGGATCCCCGGGTACCGA |
| | (SEQ ID NO: 296) |
| Ps13b-pre-crRNA-AAA-R | cgaggTGCCTTTACTACATG |
| | TGTGATCTGATAACCTTTTG |
| | TTGTAATAGCCCCCAAAACT |
| | (SEQ ID NO: 297) |
| Ps13b-pre-crRNA-CCC-R | cgaggTGCCTTTACTACATG |
| | TGTGATCTGATAACCTGGGG |
| | TTGTAATAGCCCCCAAAACT |
| | (SEQ ID NO: 298) |
| Ps13b-pre-crRNA-GGG-R | cgaggTGCCTTTACTACATG |
| | TGTGATCTGATAACCTCCCG |
| | TTGTAATAGCCCCCAAAACT |
| | (SEQ ID NO: 299) |
| Ps13b-pre-crRNA-UUU-R | cgaggTGCCTTTACTACATG |
| | TGTGATCTGATAACCTAAAG |
| | TTGTAATAGCCCCCAAAACT |
| | (SEQ ID NO: 300) |
| Hfq-Ndel-F | CCCATATGGCTAAGGGGCAA |
| | TCTTTACAAGATC |
| | (SEQ ID NO: 301) |
| Hfq-6H-Hind3-R | GGAAGCTTAGTGATGGTGAT |
| | GGTGATGTTCGGTTTCTTCG |
| | CTGTCCTGTTGCG |
| | (SEQ ID NO: 302) |
| Hfq-Tyr25TAG-F | AGTTTCTATTTAGTTGGTGA |
| | ATGGTATTAAGCTG |
| | (SEQ ID NO: 303) |
| Hfq-Tyr25TAG-R | GGAACACGTTCCCGACGC |
| | (SEQ ID NO: 304) |
| Hfq-Ile30TAG-F | GGTGAATGGTTAGAAGCTGC |
| | AAG |
| | (SEQ ID NO: 305) |
| Hfq-Ile30TAG-R | AAATAAATAGAAACTGGAAC |
| | AC |
| | (SEQ ID NO: 306) |
| Hfq-Thr49TAG-F | GTTGAAAAACTAGGTCAGCC |
| | AGATG |
| | (SEQ ID NO: 307) |
| Hfq-Thr49TAG-R | AGGATCACGAACTGATCAAA |
| | AG |
| | (SEQ ID NO: 308) |
| rnpB-qPCR-F | CGGGCGGAGGGGAGGAAAG |
| | (SEQ ID NO: 309) |
| rnpB-qPCR-R | ATCGGCGGTTTGCTCTCTGT |
| | TG |
| | (SEQ ID NO: 310) |
| rpoS-qPCR-F | CATCCTGGCCGATGAAAAA |
| | (SEQ ID NO: 311) |
| rpoS-qPCR-R | TTGACGATGCTCTGCTTCAT |
| | ATC |
| | (SEQ ID NO: 312) |
| rpoS-XL-RT | CTCCGTTCTCATCAAATTCC |
| | GC |
| | (SEQ ID NO: 313) |
| ptsG-xL-RT | GACGGAACCGCCTGC |
| | (SEQ ID NO: 314) |
| rand103Tr3 adapter | /5PHOS/NNNNNNNNNNAGAT |
| | CGGAAG |
| | AGCACACGTCTG/3SPC3/ |
| | (SEQ ID NO: 315) |
| pBADr-eCLIP_ | CAAAACAGCCAAGCTTCAGA |
| Rand103tr3-pR | CGTGTGCTCTTCCGATCT |
| | (SEQ ID NO: 316) |
| pBADf-rpoS_xL-pF | AAGAAGGAGATATACATCAG |
| | CGTATTCTGACTCATAAGGT |
| | GGCTCC |
| | (SEQ ID NO: 317) |
| pBADf-ptsG-xL-pF | AAGAAGGAGATATACATCTG |
| | CGATAGGCAGTACGG |
| | (SEQ ID NO: 318) |
| pcDNA31-Hind3- | GCTGGCTAGCGTTTAAACTT |
| PsCas13b-F | AAGCTGCCACCATGAACATC |
| | CCCGCTCTGGTGGAAAAC |
| | (SEQ ID NO: 319) |
| pcDNA31-BamH1- | TTCCACCACACTGGACTAGT |
| PsCas13b-R | GGATCTTAGTGATGGTGATG |
| | GTGATGGGCATAGTCG |
| | (SEQ ID NO: 320) |
| pc43-Ps13b- | AAAGGACGAAACACCCTGGC |
| crACTB1-F | GGCGGGTGTGGACGGGCGGC |
| | GGATCGTTGTGGAAGGTCCA |
| | (SEQ ID NO: 321) |
| pc43-Ps13b- | AAAGGACGAAACACCGGTTT |
| crNEAT1-1-F | TCAGATCACACATGTAGTAA |
| | AGGCAGTTGTGGAAGGTCCA |
| | (SEQ ID NO: 322) |
| pc43-Ps13b- | AAAGGACGAAACACCAATTG |
| crNEAT1-2-F | TTTGCATCATCCCCAAGTCA |
| | TTGGTGTTGTGGAAGGTCCA |
| | (SEQ ID NO: 323) |
| pc43-Ps13b-cr-R | AATTCGAGCTCGGTACCAAA |
| | AAAGTTGTAATAGCCCCTCA |
| | AAACTGGACCTTCCACAAC |
| | (SEQ ID NO: 324) |
| HsGAPDH-qPCR-F | CTGGGCTACACTGAGCACC |
| | (SEQ ID NO: 325) |
| HsGAPDH-qPCR-R | AAGTGGTCGTTGAGGGCAAT |
| | G |
| | (SEQ ID NO: 326) |
| HsACTB-qPCR-F | CTGGCACCACACCTTCTACA |
| | (SEQ ID NO: 327) |
| HsACTB-qPCR-R | GAGGCGTACAGGGATAGCAC |
| | (SEQ ID NO: 328) |
| HsNEAT1-qPCR-F | TCCTCCTGGTGGCCAAGACA |
| | GC |
| | (SEQ ID NO: 329) |
| HsNEAT1-qPCR-R | GCTAAGGGGCAGCGAAGGAT |
| | GC |
| | (SEQ ID NO: 330) |
| Mm-SFYRS-Spel-F | AACAGGAGGAATTACTAGTA |
| | TGGATAAAAAGCCTTTG |
| | (SEQ ID NO: 331) |
| Mm-SFYRS-SalI-R | GATGATGATGATGATGGTCG |
| | ACTTACAGGTTAGTAGAA |
| | (SEQ ID NO: 332) |
| Ma-PyIRS-SpeI-F | TAACAGGAGGAATTACTAGT |
| | ATGACCGTGAAGTAC |
| | (SEQ ID NO: 333) |
| Ma-SFYRS-R1 | CAGGTTTGGCGCCAGC |
| | (SEQ ID NO: 334) |
| Ma-SFYRS-F2 | GCTGGCGCCAAACCTGTTGA |
| | GCGTGGCTCGTGACCTGCG |
| | (SEQ ID NO: 335) |
| Ma-SFYRS-R2 | CAGCATGGTGAACTCC |
| | (SEQ ID NO: 336) |
| Ma-SFYRS-F3 | GGAGTTCACCATGCTGGCTC |
| | TGATGGATATGGGTCCGC |
| | (SEQ ID NO: 337) |
| Ma-SFYRS-R3 | CGGCTCATGCACGTC |
| | (SEQ ID NO: 338) |
| Ma-SFYRS-F4 | CGTGCATGAGCCGACAAGCG |
| | GTGCTGGTTTTG |
| | (SEQ ID NO: 339) |
| Ma-PylRS-SalI-R | TGATGATGATGATGGTCGAC |
| | TTAATTGATTTTGGCACCA |
| | (SEQ ID NO: 340) |
| Ma-PyIT(6)-F | CTAGCATAGCGGGGTTCGAC |
| | GCCCCGGTCTCTCGCCAAAT |
| | TCGAAAAGCCTGC |
| | (SEQ ID NO: 341) |
| Ma-PylT(6)-R | GTTTTAGAGACCCGCTGGTC |
| | GCCGGACCGTCCCCCAATGC |
| | GGGGCGCATCT |
| | (SEQ ID NO: 342) |
| Ma-PylT(wt)-F | AAAACCTAGCCAGCGGGGTT |
| | CGACG |
| | (SEQ ID NO: 343) |
| Ma-PylT(wt)-R | AGAGACCCGCTGGTCGCC |
| | (SEQ ID NO: 344) |
| HR-MmPyIRS-Nhel-F | GGGAGACCCAAGCTGGCTAG |
| | CGCCACCATGGATAAAAAG |
| | (SEQ ID NO: 345) |
| HR-MmPyIRS-NotI-R | CTGATCAGCGGGTTTAAAGC |
| | GGCCGCTTACAGGTTAGTAG |
| | AA |
| | (SEQ ID NO: 346) |
| U6-F1 | GGGCAGGAAGAGGGCCT |
| | (SEQ ID NO: 347) |
| U6-R1 | CGCCGGACCGTCCCCCGGTG |
| | TTTCGTCCTTTC |
| | (SEQ ID NO: 348) |
| Ma-PylT(6)-F2 | GAAAGGACGAAACACCGGGG |
| | GACGGTCCGGCG |
| | (SEQ ID NO: 349) |
| Ma-Py1T(6)-R2 | CTCTTCCTGCCCCTCGACAA |
| | AAAACGAGAGACCGGGG |
| | (SEQ ID NO: 350) |
| HR-pNEU-tRNA- | GGGGATCGGGTCTAGACTCG |
| XbaI-XhoI-F | AGGGGCAGGAAGAGGGCCT |
| | (SEQ ID NO: 351) |
| HR-pNEU-tRNA-SalI-R | GTGCCACCTGACGTCGACAA |
| | AAAACGAGAGACCGGGG |
| | (SEQ ID NO: 352) |
| HR-Ma-SFYRS-NheI-F | GAGACCCAAGCTGGCTAGCG |
| | CCACCATGACCGTGAAG |
| | (SEQ ID NO: 353) |
| HR-Ma-SFYRS-NotI-R | GATCAGCGGGTTTAAAGCGG |
| | CCGCTTAATTGATTTTGGCA |
| | C |
| | (SEQ ID NO: 354) |
| pc31-Hd3-YTHDF1-F | GCTGGCTAGCGTTTAAACTT |
| | AAGCTGCCACCATGTCGGCC |
| | ACCAGCGTGG |
| | (SEQ ID NO: 355) |
| YTHDF1-2xstrep-R | TGAGGGTGAGACCACGCACT |
| | TTGTTTGTTTCGACTCTGCC |
| | GTTCCT |
| | (SEQ ID NO: 356) |
| 2xstrep-tag_Hs-F | AGTGCGTGGTCTCACCCTCA |
| | ATTTGAGAAGGGTGGTGGGT |
| | CAGGTGGGGGTTCAGGGGGA |
| | (SEQ ID NO: 357) |
| 2xstrep-tag_Hs-R | CCCGCCACCCTTTTCAAACT |
| | GGGGATGACTCCATGCTGAT |
| | CCCCCTGAACCCCCACCTGA |
| | (SEQ ID NO: 358) |
| pc31-HA-strep-F | AGTTTGAAAAGGGTGGCGGG |
| | TACCCATACGATGTTCCAGA |
| | TTACGCTTAAGATCCACTAG |
| | TCCAGTGTGGTGGAATT |
| | (SEQ ID NO: 359) |
| pc31-Nde1-R | TTTAAACGCTAGCCAGCTTG |
| | GGTCTCCCTATAGTGAGTCG |
| | TATTAAT |
| | (SEQ ID NO: 360) |
| YTHDF1-Y397TAG-F | CAAGAGCTAGTCTGAGGACG |
| | ACATCCACCGCT |
| | (SEQ ID NO: 361) |
| YTHDF1-Y397TAG-R | CCTCAGACTAGCTCTTGATG |
| | ATGAACACACGC |
| | (SEQ ID NO: 362) |
| ACTB-m6A-1-RT | CAATCAAAGTCCTCGGCC |
| | (SEQ ID NO: 363) |
| DICER1-m6A-1-RT | GAAAGGTTCTTTTGTTGGCT |
| | G |
| | (SEQ ID NO: 364) |
| JUN-m6A-1-RT | GAGTTCATCTGTAGGCTCAG |
| | C |
| | (SEQ ID NO: 365) |
| pBADf-ACTB-m6A-1-pF | AAGAAGGAGATATACATCCG |
| | TTCCAGTTTTTAAATCCTGA |
| | GTCAAGC |
| | (SEQ ID NO: 366) |
| pBADf-DICER1-m6A-1-pF | AAGAAGGAGATATACATGGG |
| | CCTTTTCCCGATCAGTCC |
| | (SEQ ID NO: 367) |
| pBADf-JUN-m6A-1-pF | AAGAAGGAGATATACATGAG |
| | TACTACAGAAGCAATCTACA |
| | GTCTCTATTGCAG |
| | (SEQ ID NO: 368) |

### Example 2

Recent advances in genetic code expansion have led to an increase in the development of bioreactive unnatural amino acids. Bioreactive unnatural amino acids (Uaas) are of particular interest due to their use in the development of covalent proteins for research and therapeutics. Uaas that react with a variety of nucleophilic canonical amino acids instead of just one lead to an increase in the diversity of proteins that can be targeted or studied using a covalent Uaa strategy. Aryl fluorosulfates are of particular interest in the design of bioreactive Uaas due to their tunable reactivity, relative stability in physiological conditions, and their selectivity for certain nucleophilic residues. Several Uaas containing aryl fluorosulfate moieties have been developed previously. Herein, we describe a novel aryl fluorosulfate Uaa, meta-fluorosulfate-L-tyrosine, or mFSY, which can crosslink with lysine, tyrosine, and histidine residues on target proteins in a selective manner. mFSY is easy to synthesize, has robust incorporation in E. coli and mammalian cells, and allows for the development of covalent protein binders that can selectively and efficiently target proteins of interest. As proof, we incorporated mFSY into different protein binders targeting HER2 and EGFR, proteins of particular interest in various cancers, and showed robust crosslinking. Our results show potential for the use of mFSY in the development of covalent biological therapeutics, a growing field that promises to alter the way we treat cancers and other diseases. When combined with previously developed aryl fluorosulfate-containing Uaas, or utilized on its own, mFSY is a powerful tool in the arsenal of bioreactive Uaas that can be used in protein engineering for a wide range of purposes.

Engineering novel functions into proteins is a powerful tool. Utilizing genetic code expansion to engineer proteins with crosslinking capabilities greatly impacts our ability to develop potent therapeutics and valuable research tools. (Refs 1-4). In recent years, there has been a large push in the field to develop bioreactive unnatural amino acids (Uaas) for these reasons. (Refs. 5-9). However, designing bioreactive Uaas is difficult. Uaas that are too reactive can be nonspecific, reacting with multiple resides of different proteins in the cell, causing off-target effects and even potentially leading to cell toxicity. For this reason, the reactivity and selectivity of the crosslinking Uaa have to be finely tuned.

One of the most ideal reactive warheads to use in bioreactive Uaa development is an aryl fluorosulfate. (Ref 10). Aryl fluorosulfates are not highly reactive moieties, but rather selectively reactive with nucleophilic residues only when brought into close proximity. Aryl fluorosulfates are also stable in physiological conditions and can react with nucleophilic residues via the SuFEx click reaction in water, at physiological pH, without any catalyst or additive needed. The byproducts of the SuFEx reaction are also nontoxic to cells. Our lab has previously developed the first aryl fluorosulfate-containing Uaa, fluorosulfate-L-tyrosine, or FSY, which we have shown to be an effective and efficient tool for protein crosslinking in vitro and in cells. (Ref 11). FSY has been used to study various protein signaling pathways and protein-protein interactions in cells, and has recently been shown to be a promising tool for the development of covalent biological therapeutics. Most recently, we developed fluorosulfonyloxybenzoyl-L-lysine, or FSK, another aryl fluorosulfate-containing Uaa which complements FSY with its broader radius of reactivity. (Ref 12). Our lab has thus far shown that aryl fluorosulfate-containing Uaas like FSY and FSK are great tools in the arsenal of bioreactive Uaas that can be used for protein engineering, biochemical research, and therapeutic development.

Here, we describe the development of another aryl fluorosulfate Uaa, meta-fluorosulfate-L-tyrosine (mFSY), which can be used alongside FSY and FSK to crosslink proteins for various purposes. mFSY bears the fluorosulfate group on the meta position of the aryl ring and reacts in a similar manner to FSY. We showed that, when incorporated into engineered proteins such as nanobodies and affibodies, and expressed in both E. coli and mammalian cells, mFSY exhibits robust crosslinking with different proteins of interest at various sites. mFSY was used to crosslink an affibody dimer to its target, HER2. We then generated several covalent nanobodies that irreversibly bound their target proteins, HER2 and EGFR. Furthermore, we showcased the first example of bioreactive Uaa incorporation being used to develop a covalent Fab that crosslinks its target receptor. The data shown here collectively indicates that mFSY is a new Uaa that will be useful in therapeutic development and biochemical research.

### Results

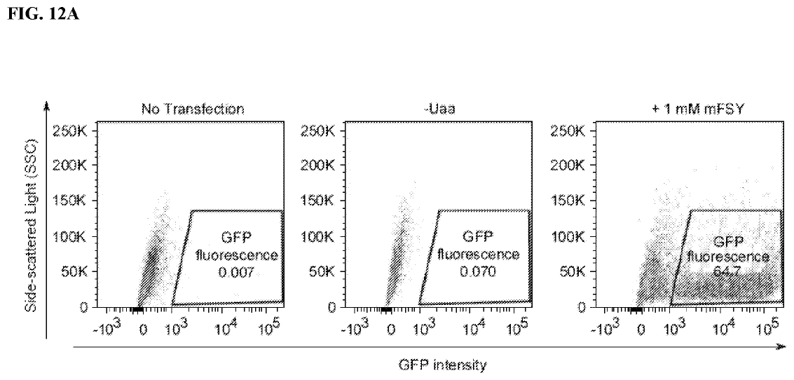

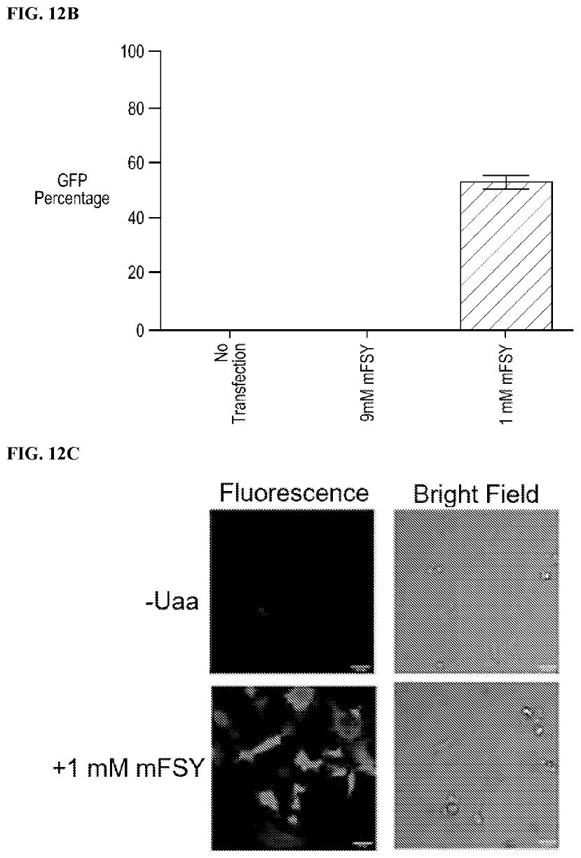

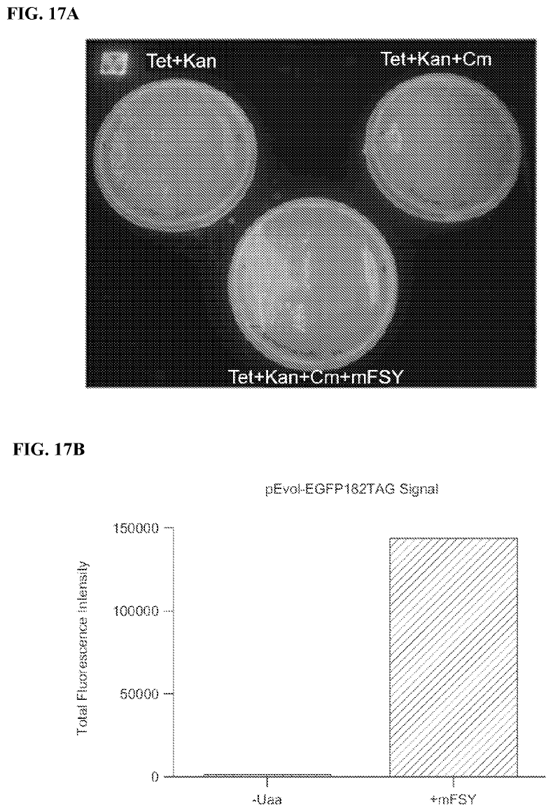

mFSY synthesis and synthetase selection. We designed mFSY to complement our already developed Uaas FSY and FSK. The meta position of the fluorosulfate has the same relative reactivity as the para position of the fluorosulfate group of FSY, making mFSY a good substitute for FSY or potentially a tool to be used in tandem with FSY. We screened for an orthogonal synthetase/tRNA pair for mFSY using a previously described screening method. Using a Methanosarcina mazei PyIRS synthetase mutant library previously constructed in our lab, we identified a hit (L305M/1322T/N346G), herein called mFSYRS, which incorporated mFSY into EGFP with high efficiency (FIGS. 17A-17B). (Refs 13 14). Incorporation fidelity was validated via intact and peptide mass spectrometry analysis of the affibody dimer dZ_{HER2} with mFSY incorporated at position 37 (dZfER2-37mFSY) (data not shown). We further tested the incorporation of mFSY in mammalian cells (FIG. 12). mFSYRS was cloned into a mammalian expression vector then transfected into HeLa-EGFP (182TAG) cells, a stable cell line expressing EGFP with a TAG codon at permissive site 182. When 1 mM of mFSY was added to HeLa-EGFP (182TAG) cells transfected with mFSYRS, FACS data showed greater than 50% incorporation of mFSY over three replicates, compared with negligible rates of misincorporation of canonical amino acids in samples without mFSY added (FIGS. 12A-12B). Microscopy images further confirm the expression of full length EGFP in HeLa-EGFP (182TAG) cells transfected with mFSYRS and treated with mFSY as compared with untreated cells (FIG. 12C).

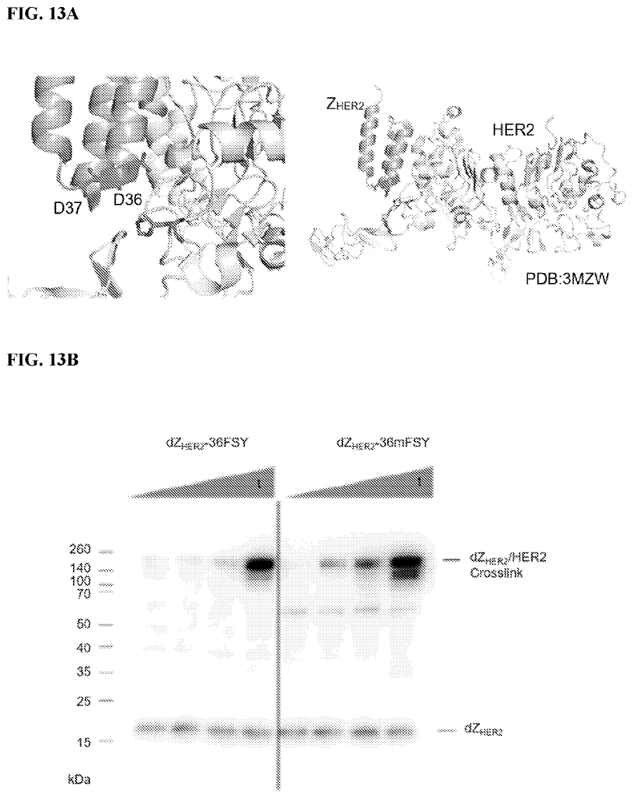

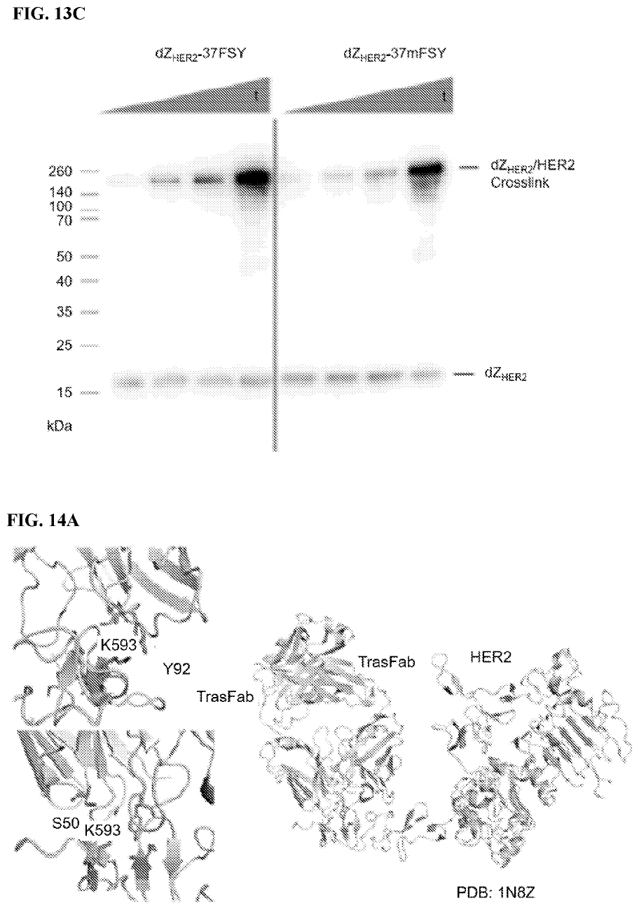

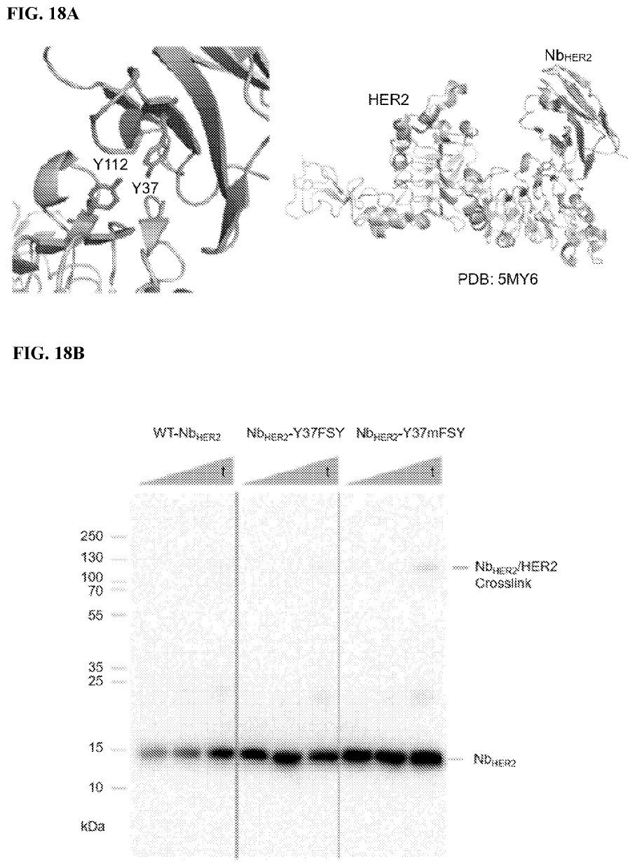

mFSY incorporated into engineered protein binders enables the covalent crosslinking of target proteins HER2 and EGFR. To prove mFSY as a new bioreactive Uaa, we incorporated it into a number of different engineered protein binders targeting either HER2 receptor or EGFR and showed crosslinking between the mutant proteins and their targets. First, we incorporated mFSY into the affibody dimer, dZ_{HER2}, at two sites, D36 and D37, which were shown via crystal structure to be proximal to the nucleophilic residue H490 on the HER2 receptor (FIG. 13A). (Ref 15). At both sites, mFSY was shown to efficiently crosslink with the HER2 receptor in a time-dependent manner (FIGS. 13B-13C). When compared with dZ_{HER2} mutants incorporating FSY at sites 36 and 37, mFSY mutants crosslinked HER2 at a similar rate, suggesting that mFSY is as efficient as FSY for certain protein crosslinking purposes. When incorporated into a different engineered protein, nanobody Nb_{HER2}, mFSY led to detectable crosslinking of HER2 receptor, albeit less efficiently than the dZ_{HER2} mutant proteins (FIGS. 18A-18B). (Ref 16). FSY incorporation into the same site of Nb_{HER2}, however, shows no detectable crosslinking between the mutant nanobody and HER2.

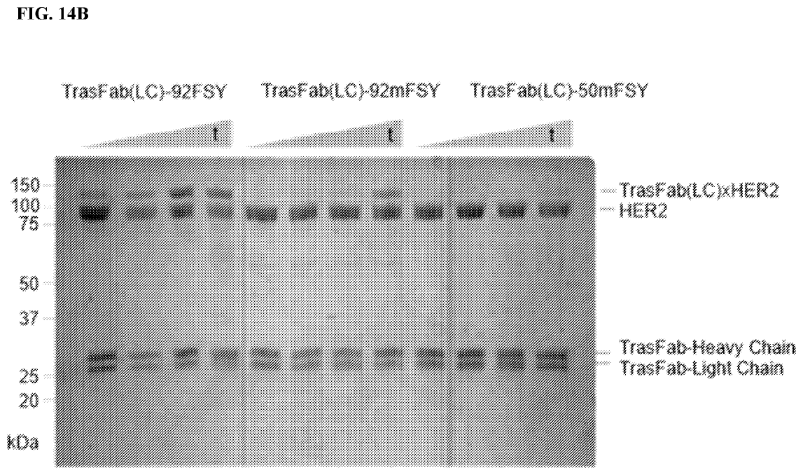

To finish out the study of HER2-targeting protein binders, we incorporated mFSY into the Trastuzumab Fab, TrasFab (FIG. 14). Trastuzumab is a well-known antibody targeting HER2. The TrasFab, though stable and tight-binding, has a significantly worse therapeutic effect when used to treat HER2 positive cancer cells as compared with full length trastuzumab. (Ref 17). By incorporating a covalent Uaa into TrasFab, we could improve the pharmacokinetics of TrasFab and provide a better overall therapeutic effect for TrasFab. mFSY was incorporated into sites S50 and Y92 on the light chain of TrasFab; site Y92 showed robust crosslinking when incubated with the extracellular domain of HER2 receptor (FIG. 14B). Similar time-dependent crosslinking bands were seen via SDS-PAGE analysis for the TrasFab(LC)-92FSY mutant incubated with HER2. TrasFab(LC)-50mFSY showed less efficient crosslinking with HER2 receptor, showcasing that mFSY-mediated crosslinking is site-dependent (FIG. 14B). This is the first example of a Fab that has been engineered to crosslink the receptor that it targets via a bioreactive Uaa.

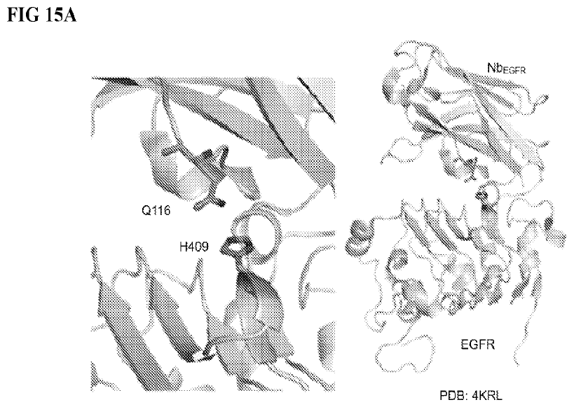

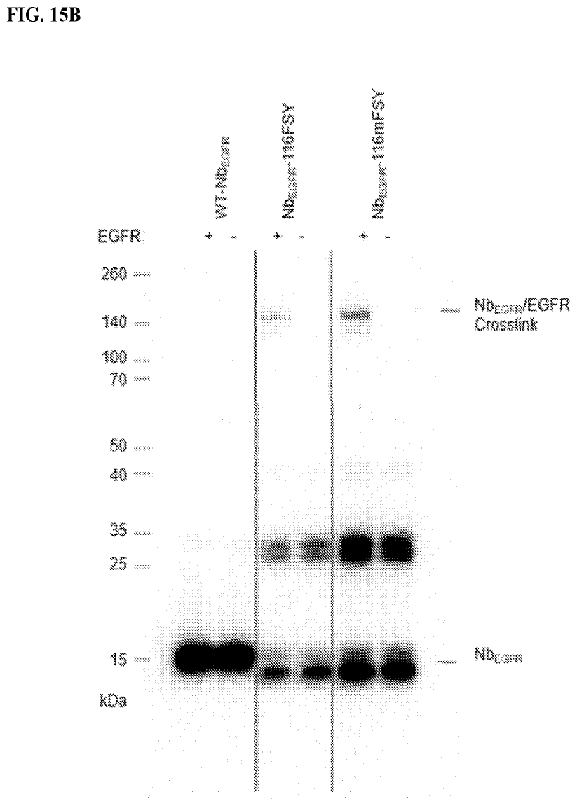

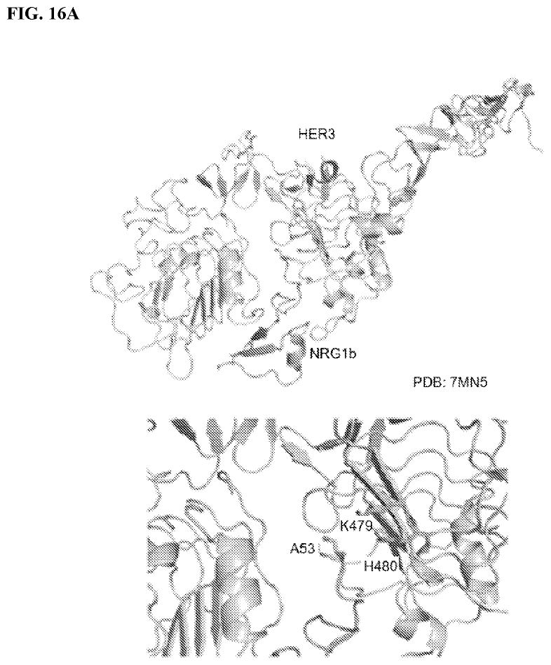

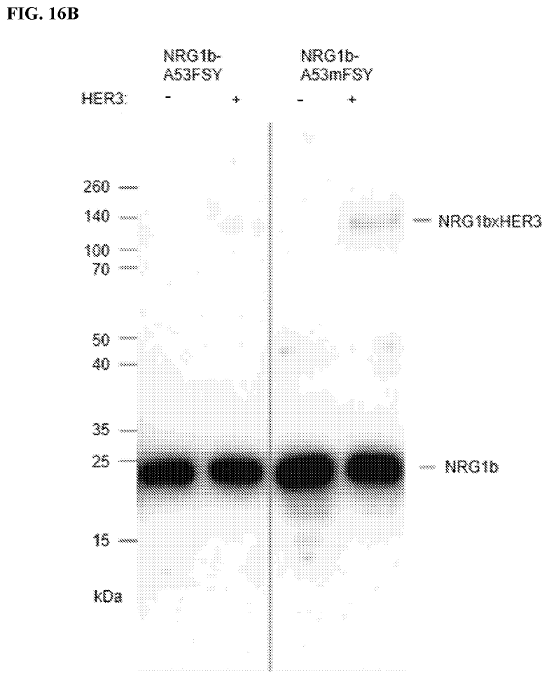

We continued to test the crosslinking capabilities of mFSY by incorporating it into other protein binders that targeted different members of the ErbB family of receptors. We incorporated mFSY into site Q116 of a nanobody targeting EGFR (FIG. 15). (Ref 18). Q116mFSY mutant protein showed robust crosslinking with EGFR at a comparable rate and efficiency as the Q116FSY mutant (FIG. 15B). We then incorporated mFSY into site A53 of Neuregulin 1β (NRG1b), which binds to HER3 and activates its signaling pathways. (Ref 19). NRG1b-A53mFSY showed crosslinking with the extracellular domain of HER3, however, FSY incorporated at this same site did not (FIG. 161B). While both of these results show that mFSY is an efficient bioreactive Uaa that can be incorporated into various protein binders and used to target different proteins of interest, our NRG1b-HER3 binding study further showcases how our new fluorosulfate-containing Uaa mFSY can be used alongside of or instead of other established Uaas to achieve crosslinking that was otherwise not possible.

### DISCUSSION

mFSY is set to be another tool for protein research and therapeutic development. Our data shows robust incorporation into various proteins in E. coli and mammalian cells. We see robust crosslinking between different engineered proteins containing mFSY and their targets at various sites and with different nucleophiles. Most notably, we show the first case of a Fab with a covalent Uaa crosslinking to its target protein. With robust crosslinking, mFSY will be useful for therapeutic development, particularly for covalent biological therapeutics. Our lab has shown the potential for such covalent therapeutics in the treatment of cancer; expanding the arsenal of bioreactive Uaas will further expand our ability to develop more of these promising therapeutics in the future. (Ref 3).

We have shown that mFSY has comparable crosslinking efficiency to FSY. Both Uaas can potentially be used interchangeably to crosslink the same proteins at the same sites. FSY and mFSY can also be used together simultaneously in a protein via dual Uaa incorporation to target two different sites of a protein of interest or a protein complex of interest. Our data shows that, while the two Uaas are generally interchangeable, some incorporation sites do lead to differences in crosslinking efficiencies, thus one Uaa can potentially be used to increase crosslinking efficiency when the other does not work as robustly. Given the three aryl fluorosulfate-containing Uaas our lab has developed, FSY, FSK, and mFSY have the potential to be useful tools for biochemical study and therapeutic development.

### Materials and Methods

Chemical Synthesis of Meta-FSY (mFSY)

Synthesis of aryl fluorosulfates was based on recent methods to synthesize sulfur (IV) fluorides using [4-(acetylamino)phenyl]imidodisulfuryl difluoride (AISF) reagent. (Zhou et al, Org Lett 2018, 20 (3), 812-815).

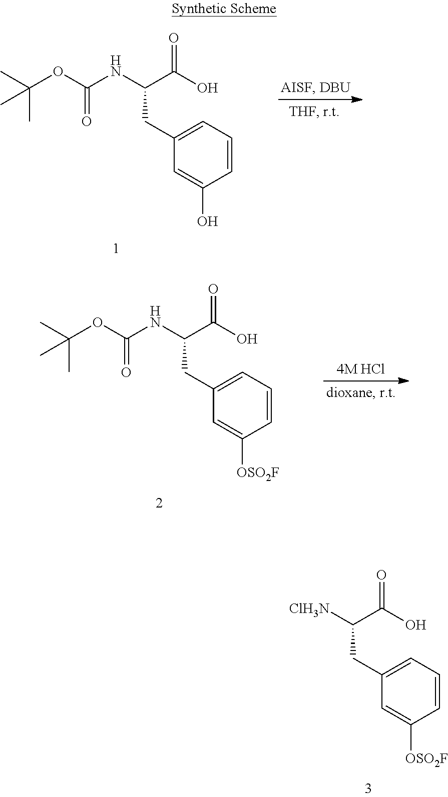

Synthesis of (S)-2-amino-3-(3-((fluorosulfonyl)oxy)phenyl)propanoic acid (3, mFSY). To a 100 mL round-bottom flask were added Boc-(S)-2-Amino-3-(3-hydroxyphenyl)propionic acid (1, 1.15 g, 4.09 mmol) and [4-(acetylamino)phenyl]imidodisulfuryl difluoride (AISF) reagent (1.54 g, 4.90 mmol, 1.2 equiv). The mixture was dissolved in 25 mL anhydrous tetrahydrofuran and 1,8-diazabicyclo[5.4.0]undec-7-ene (1.37 mL, 9 mmol, 2.2 equiv) was added dropwise while stirring. The solution was then stirred at room temperature for 30 minutes. The reaction was then diluted with 50 mL ethyl acetate and washed with 1 M HCl (100 mL×2) and brine (100 mL×1). The organic fraction was dried with anhydrous sodium sulfate and concentrated under vacuum. The crude product was then purified by column chromatography using MeOH:CH2Cl2 (1:200). The product, (S)-2-((tert-butoxycarbonyl)amino)-3-(3-((fluorosulfonyl)oxy)phenyl)propanoic acid, was isolated as a white solid (2, 0.774 g, 2.13 mmol, 52%).

(S)-2-((tert-butoxycarbonyl)amino)-3-(3-((fluorosulfonyl)oxy)phenyl)propanoic acid (2, 0.774 g, 2.13 mmol) was added to a scintillation vial and dissolved in 4 M HCl in dioxane (10 mL). The reaction was stirred overnight. The resultant solid was filtered off and washed with cool ether (10 mL×2) affording the product mFSY-HCl as a white solid (3, 554 mg, 1.85 mmol, 87%). ¹H NMR (400 MHz, D₂O): δ (ppm) 7.62-7.58 (t, J=16.0 Hz, 1H), 7.50-7.45 (m, J=19.2 Hz, 3H), 4.36-4.33 (t, J=13.2, 1H), 3.45-3.29 (m, J=61.2 Hz, 2H). ¹³C NMR (400 MHz, D₂O): δ (ppm) 171.4, 150.1, 137.3, 131.3, 130.0, 121.8, 120.4, 54.1, 35.3. HR-ESI (+) m/z: calculated for C₉H₁₀FNO₅S (M+H)⁺, 264.0264; found 264.0351

Library Construction and mFSYRS Mutant Selection

The pBK-TK3 mutant library of MmPyIRS was constructed using the new small-intelligent mutagenesis approach, which uses a single codon for each amino acid and thus allows a greater number of residues to be mutated simultaneously. The following residues of MmPyIRS were mutated: 302NYT, 305WTG, 306WTG/TAC, 309KYA, 322AYA, 346NDT/VMA/ATG/TGG, 348NDT/VMA/ATG/TGG, 384TTM/TAT, 401VTT, 417NDT/VMA/ATG/TGG, using the procedures described by Liu et al, J Am Chem Soc 2021, 143 (27), 10341-10351; and Lacey et al, Chembiochem 2013, 14 (16), 2100-2105). The selection was performed as described by Wang et al, J Am Chem Soc 2018, 140 (15), 4995-4999. Briefly, pBK-TK3 library was transformed into DH10b-pRep positive selection reporter cells via electroporation. The cells were then plated onto an LB-agar selection plate containing 1 mM FSY, 12.5 μg/mL of tetracycline (Tet), 25 μg/mL of kanamycin (Kan), and 75 μg/mL of chloramphenicol (Cm). The selection plate was incubated at 37° C. for 72 h and then stored at 4° C. Colonies showing green fluorescence was picked and streaked on a fresh LB-agar plate containing either Tet12.5Kan25Cm100 or Tet12.5Kan25Cm100+1 mM mFSY. After 24 h of incubation at 37° C., 2 clones present mFSY-dependent fluorescence and growth were considered as hits and further characterized. The pBK plasmids encoding PyIRS mutants were extracted by miniprep and then separated from reporter plasmids by DNA gel electrophoresis. The purified pBK plasmids were analyzed by Sanger-sequencing.

Incorporation of mFSY into EGFP-182TAG

pBAD-EGFP-182TAG was co-transformed with pEVOL-mFSYRS into DH10b and plated on LB agar plate supplemented with 100 μg/mL ampicillin and 34 μg/mL chloramphenicol. A single colony was picked and inoculated into 1 mL 2×YT (5 g/L NaCl, 16 g/L Tryptone, 10 g/L Yeast extract). The cells were left grown at 37° C., 220 rpm, for 16h. The cells were then diluted to an OD Of 0.6 in fresh 2×YT supplemented with relevant antibiotics, with or without 1 mM mFSY. The cells were then induced with 0.2% Arabinose at either 30° C. for 6 hr. The fluorescence intensity was measured with a plate reader (excitation at 485 nm, emission at 528 nm) and normalized to OD at 600 nm

General Incorporation of mFSY into Proteins for Expression and Purification

For the incorporation of mFSY into dZHER2, NbHER2, TrasFab, NbEGFR, and NRG1b, the procedure of transformation is the same as described above. After transformation, a single colony was picked and left grown at 37° C., 220 rpm for 16h. Next morning, the cell culture was diluted 100 times and then regrown to an OD 0.6-0.8 in 100 mL scale, with good aeration and the relevant antibiotic selection. Then the medium was added with 0.2% arabinose (and 1 mM IPTG for TrasFab and NRGTb) with or without 1 mM mFSY, and the expression were carried out at 18° C., 220 rpm for 18 hr, 18° C., or 25° C. The IMAC chromatography was used for protein purification and the procedure was described by Li et al, Cell 2020, 182 (1), 85-97.e16. TrasFab was purified on the Äkta Pure FPLC protein purification system using an HiTrap® Protein A column. Procedure was described by Hornsby et al, Mol Cell Proteom Mcp 2015, 14 (10), 2833-2847.

In Vitro Cross-Linking of dZHER2, NbHER2, TrasFab, NbEGFR, and NRGTb with HER2, EGFR, and HER3

Recombinant extra-cellular domain (ECD) of HER2 receptor was purchased from Abcam (Cat #ab168896); EGFR-ECD receptor was purchased from Abcam (Cat #ab155726); HER3-ECD receptor was purchased from Acros biosystems (Cat #ER3-H5259). Purified 1 μM dZHER2, NbHER2, or TrasFab was incubated with 1 μM HER2 ECD in 20 uL 1×PBS, 7.4 at 37° C. for 16 h. Purified 3 μM NbEGFR was incubated with 500 nM EGFR in 20 uL 1×PBS, 7.4 at 37° C. for 16 h. Purified 150 nM trx-NRG1b was incubated with 500 nM HER3 in 20 uL 1×PBS, 7.4 at 37° C. for 16 h. After incubation, 4× Laemmli Sample Buffer (Bio Rad, Cat #161-0747) was added into the incubation and heated at 95 C for 10 min. The samples were separated on SDS-PAGE and either analyzed by Coomassie blue staining or immunoblotted with 1:10000 anti-his monoclonal antibody (Proteintech #HRP66005).

### Mass Spectrometry

Mass spectrometric measurements were performed as described by Liu et al, J Am Chem Soc 2017, 139 (9), 3430-3437. Briefly for electrospray ionization mass spectrometry, mass spectra of intact proteins were obtained using a QTOF Ultima (Waters) mass spectrometer, operating under positive electrospray ionization (+ESI) mode, connected to an LC-20AD (Shimadzu) liquid chromatography unit. Protein samples were separated from small molecules by reverse phase chromatography on a Waters Xbridge BEH C4 column (300 Å, 3.5 μm, 2.1 mm×50 mm), using an acetonitrile gradient from 30-71.4%, with 0.1% formic acid. Each analysis was 25 min under constant flow rate of 0.2 mL/min at RT. Data were acquired from m/z 350 to 2500, at a rate of 1 sec/scan. Alternatively, spectra were acquired by Xevo G2-S QTOF on a Waters ACQUITY UPLC Protein BEH C4 reverse-phase column (300 Å, 1.7 μm, 2.1 mm×150 mm). An acetonitrile gradient from 5%-95% was used with 0.1% formic acid, over a run time of 5 min and constant flow rate of 0.5 mL/min at RT. Spectrum were acquired from m/z 350 to 2000, at a rate of 1 sec/scan. The spectra were deconvoluted using maximum entropy in MassLynx.

For tandem mass spectrometry, analysis and sequencing of peptides were carried out using a Q Exactive Orbitrap interfaced with Ultimate 3000 LC system. Data acquisition by Q Exactive Orbitrap was as follows: 10 μL of trypsin-digested protein was loaded on an Ace UltraCore super C18 reverse-phase column (300 Å, 2.5 m, 75 mm×2.1 mm) via an autosampler. An acetonitrile gradient from 5%-95% was used with 0.1% formic acid, over a run time of 45 min and constant flow rate of 0.2 mL/min at room temperature. MS data were acquired using a data-dependent top10 method dynamically choosing the most abundant precursor ions from the survey scan for HCD fragmentation using a stepped normalized collision energy of 28, 30 35 eV. Survey scans were acquired at a resolution of 70,000 at m/z 200 on the Q Exactive. Tandem MS data was analyze on MaxQuant.

FACS Analysis of mFSY Incorporation into HeLa-GFP-182TAG

One day before transfection, 4.5×10⁴ HeLa-EGFP-182TAG reporter cells were seeded in 9 wells of a Greiner bio-one 12 well-cell culture dish containing 1 mL of DMEM media with 10% FBS, and incubated at 37° C. in a CO2 incubator. Plasmid pMP-mFSYRS (1 μg) was transfected into target cells using 9 μL polyethylenimine (PEI) transfection agent. pMP-mFSYRS plasmid was not added to three of the wells (negative control). Six hours post transfection, 1 mM mFSY was added to three wells. The remaining three wells were transfected with pMP-mFSYRS plasmid but did not have mFSY Uaa added. After incubation at 37° C. for 48 hr, cells were non-enzymatically detached from the plates using Gibco Cell Dissociation Buffer and collected by centrifugation (500 g, 5 min, r.t.). The cells were resuspended in 300 μL of FACS buffer (1×PBS, 2% FBS, 1 mM EDTA, 0.1T sodium azide, 0.28 μM DAPI) and analyzed by BD LSRFortessa™ cell analyzer.

### Fluorescence Confocal Microscopy of HeLa-GFP-182mFSY

One day before transfection, 4.5×10⁴ HeLa-EGFP-182TAG cells were seeded in a Greiner bioone CELLview glass bottom dish containing 2 mL of DMEM media with 10% FBS, and incubated at 37° C. in a CO2 incubator. Plasmid pMP-mFSYRS (2 μg) was transfected into the HeLa-EGFP-182TAG cells using 9 μL polyethylenimine (PEI) transfection agent. Six hours post transfection, 1 m mFSY was added to the media. A Hela-EGFP-182TAG cell group that was not transfected with any plasmid was used as a negative control. The cells were incubated at 37° C. for an additional 48 h post transfection and imaged with a Nikon CSU-XC Spinning Disk microscope.

Primers were synthesized and purified by Integrated DNA Technologies (IDT), and plasmids were sequenced by GENEWIZ. All molecular biology reagents were either obtained from New England Biolabs or Vazyme. His-HRP antibody were obtained from ProteinTech Group. pBAD-EGFP, pBAD-dZHER2, and pBAD-NbEGFR were used as previously described. (Refs 1, 2). The primers are shown in the Table below.

| | Oligonucleotide |
|---|---|
| Primer | Sequence (5'->3') |
| mFSRYS-BglII-F | CTAACAGGAGGAATTAG |
| | ATCTATGGATAAAAAGCCT |
| | (SEQ ID NO: 369) |
| mFSYRS-SalI-R | GATGATGATGATGATGG |
| | TCGACTTACAGGTTAGTAGAA |
| | (SEQ ID NO: 370) |
| mFSYRS-NcoI-F | TATGCCATGGATAAAAAGCC |
| | TTTG (SEQ ID NO: 371) |
| mFSYRS-NheI-R | CTATGCTAGCTTACAGGTTA |
| | GTAGA (SEQ ID NO: 372) |
| NbHER2-37TAG-For | TCTTGTGGTATGGGTTGGTA |
| | GCGTCAGAGCCCGGGT (SEQ |
| | ID NO: 373) |
| NbHER2-37TAG-Rev | CTACCAACCCATACCACAAG |
| | AGTTGAAGATATAGC (SEQ |
| | ID NO: 374) |
| TrasFab-50TAG-For | ACTAGGCATCCTTTCTCTAC |
| | TCTGGAGTCCCT (SEQ ID |
| | NO: 375) |
| TrasFab-50TAG-Rev | GAGAAAGGATGCctaGTAAA |
| | TCAGAAGCTTCGGAGCTTT |
| | (SEQ ID NO: 376) |
| TrasFab-92TAG-For | TCAGCAACATTAGACCACAC |
| | CAC (SEQ ID NO: 377) |
| TrasFab-92TAG-Rev | CAGTAATAAGTTGCGAAGTC |
| | TTC (SEQ ID NO: 378) |
| NbEGFR-116TAG-For | ATGAATACGACTACTGGGGT |
| | TAGGGTACGCAGG (SEQ ID |
| | NO: 379) |
| NbEGFR-116TAG-Rev | AACCCCAGTAGTCGTATTCA |
| | TACAGAGTGCCAT (SEQ ID |
| | NO: 380) |
| Tx-NRG1b-53TAG-For | TGTGATGTAGAGTTTCTATA |
| | AGCACCTTGGAATTGA (SEQ |
| | ID NO: 381) |
| Tx-NRG1b-53TAG- | AGAAACTCTACATCACATAA |
| Rev | TTTTGACAACGATCAC (SEQ |
| | ID NO: 382) |

### Example 3

Generate Covalent Nanobodies from mNb6 that Binds the Down-State of RBD

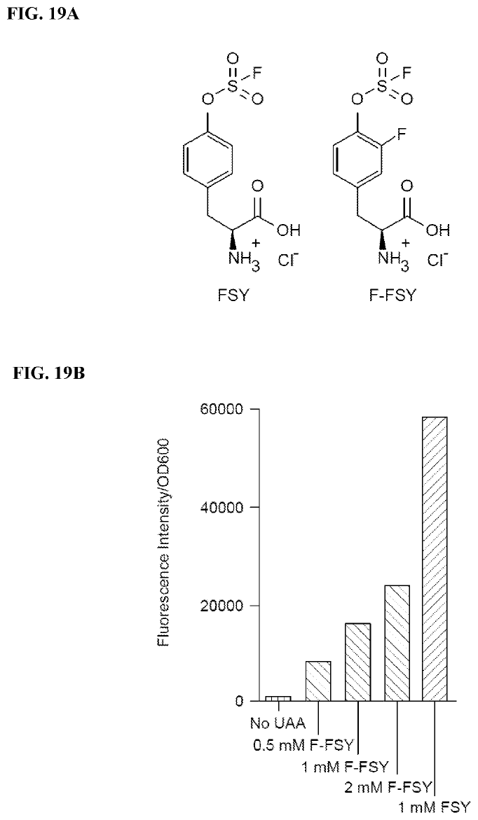

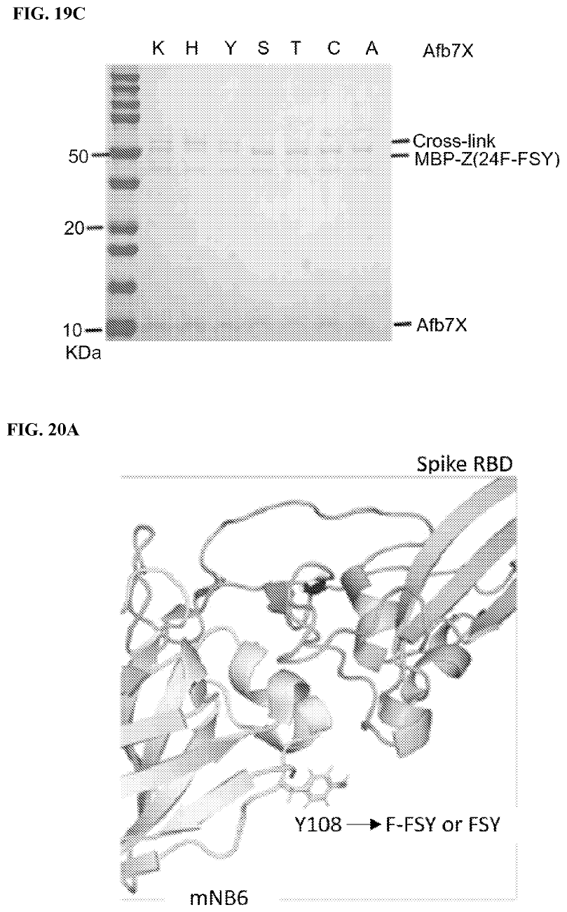

Nanobody mNb6 (SEQ ID NO:60) was isolated through screening a library against the Spike ectodomain stabilized in the prefusion conformation, and thus able to bind the Spike RBD in the down state. Schoof et al, Science 370, 1473-1479 (2020). To identify which sites of mNb6 would allow covalent crosslink of the Spike RBD, we incorporated FSY individually at 30 different sites located at the CDR1, CDR2, and CDR3 regions of mNb6. Although the crystal structure of mNB6 in complex with RBD is available, we performed this comprehensive site screening to show that for proteins with well-defined regions such as the nanobody, one can readily determine the appropriate sites for FSY cross-linking without high-throughput screening or detailed structures. We incubated 2 μM mNb6(WT) and its FSY mutant proteins with 0.5 μM Spike RBD in PBS (pH 7.4) at 37° C. for 12 h, followed with Western blot analysis (FIGS. 19A-19C). When FSY was incorporated at site 27 in CDR1, site 55 in CDR2, and sites 102-108 in CDR3, a covalent complex of mNb6 with the Spike RBD was detected, indicating that multiple sites in mNb6 would allow covalent cross-linking with the Spike RBD, which may not be obvious by only checking the crystal structures. We then performed kinetic study on the four more efficient sites on CDR3 (FIG. 19D), and found that mNB6(108FSY) showed the fastest cross-link rate. We thus chose site 108 to incorporate Uaa for subsequent experiments.

Develop FFY as a novel latent bioreactive Uaa to accelerate PERx reaction rate.

The potency of covalent protein drugs relies on the ability of latent bioreactive Uaa to form covalent bond with the target residues on the target proteins. As protein interactions are dynamic, a fast reaction rate would be beneficial to ensure covalent bond formation before protein dissociation, and can be critical to achieve complete inhibition of viral infection. Introducing electron-withdrawing groups on the aromatic ring has been reported to increase the SuFEx rates. 12 We thus envisioned that adding electron-withdrawing substituents to FSY would accelerate its proximity-enabled reaction rate when used as PERx.

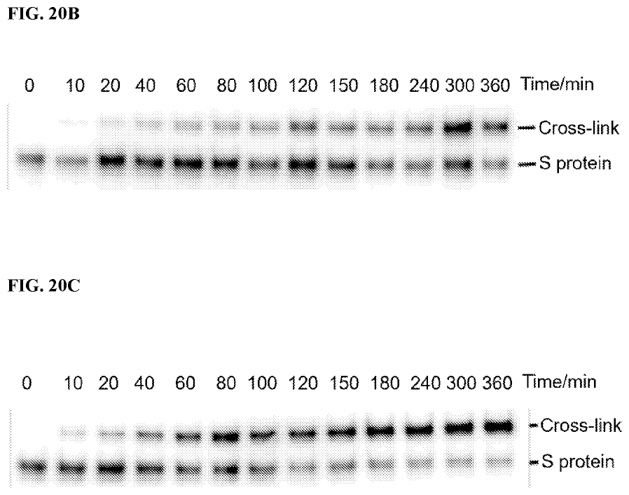

As a result, we designed and evaluated a fluoride substituted fluorosulfate-L-tyrosine, as described herein. FFY was synthesized using [4-(acetylamino)phenyl]imidodisulfuryl difluoride (AISF), followed with the deprotection of the Boc protecting group using hydrogen chloride. Zhou et al, Org. Lett. 20, 812-815 (2018). As FFY and FSY have similar structures, we reasoned that FSYRS, a pyrrolysyl-tRNA synthetase (PyIRS) mutant we previously evolved to be specific for FSY, should be able to incorporate FFY into proteins as well. Wang et al, J. Am. Chem. Soc. 140, 4995-4999 (2018). To test this, the enhanced green fluorescent protein (EGFP) gene containing a TAG codon at permissive site 182 was co-expressed with genes for tRNA^{Pyl}/FSYRS in E. coli. In the absence of FFY, no obvious fluorescence was detected; in the presence of FFY, concentration-dependent fluorescence increase was measured (FIG. 20A). We also co-expressed mNb6(108TAG) with tRNA^{Pyl}/FSYRS in E. coli, and observed that full-length mNb6 was produced in the presence of 2 mM FFY or 1 mM FSY (FIG. 20B). mNB6(WT), mNb6 (108FSY), and mNb6(108FFY) proteins were purified with Ni²⁺ affinity chromatography. Mass spectrometric analysis of the intact protein confirmed that FFY was incorporated into mNb6 at site 108 in high fidelity. A major peak observed at 13721 Da corresponds to intact mNb6(108FFY) (expected 13722.7 Da). A minor peak observed at 13702 Da corresponding to mNb6(108FFY) lacking F, suggesting a slight F elimination during mass spectrometric measurement. We also verified mNb6(WT) and mNb6 (108FSY) via mass spectrometric analysis of the intact proteins.

We next tested if FFY could improve reaction kinetics over FSY. The Spike RBD (0.5 μM) was incubated with 5 μM mNb6(108FSY) or mNb6(108FFY) in PBS (pH 7.4) at 37° C. for different duration, followed with Western blot analysis of the cross-linking. As shown in FIG. 20C, mNb6(108FFY) showed evidently faster cross-linking rate with the Spike RBD than mNB6(108FSY). Covalent cross-linking with the Spike RBD could be robustly detected within 10 min for mNb6(108FFY), while mNb6(108FSY) took about 20 min. The apparent first-order constant k_{obs} was 0.244±0.031 h⁻¹ (n=3) for mNb6(108FFY) and 0.102±0.007 h⁻¹ (n=3) for mNb6(108FSY)(FIG. 20C). Thus, FFY increased the PERx reaction rate over FSY in the mNB6 system to 240%, so we used mNb6(108FFY) for subsequent viral inhibition tests.

mNb6(108FFY) Shows Markedly Increased Potency in Neutralizing Both Pseudovirus and Authentic SARS-Cov-2 Infection

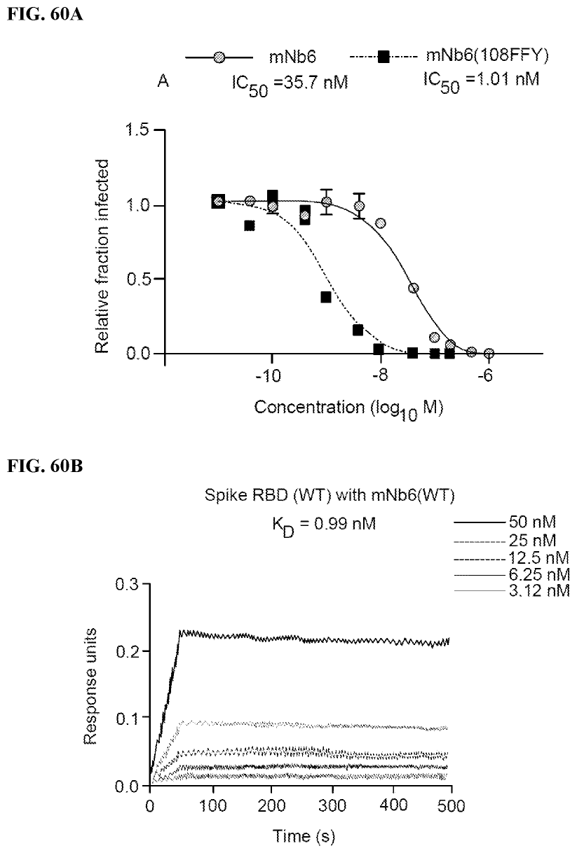

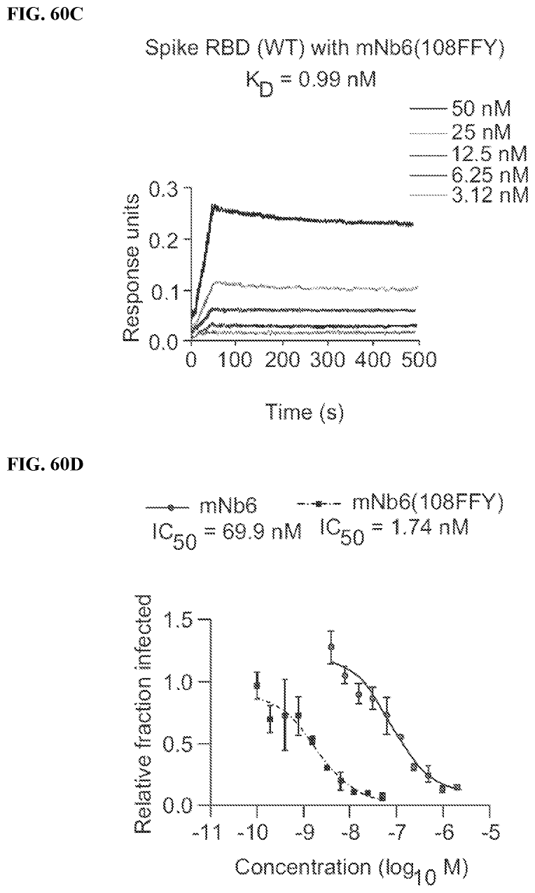

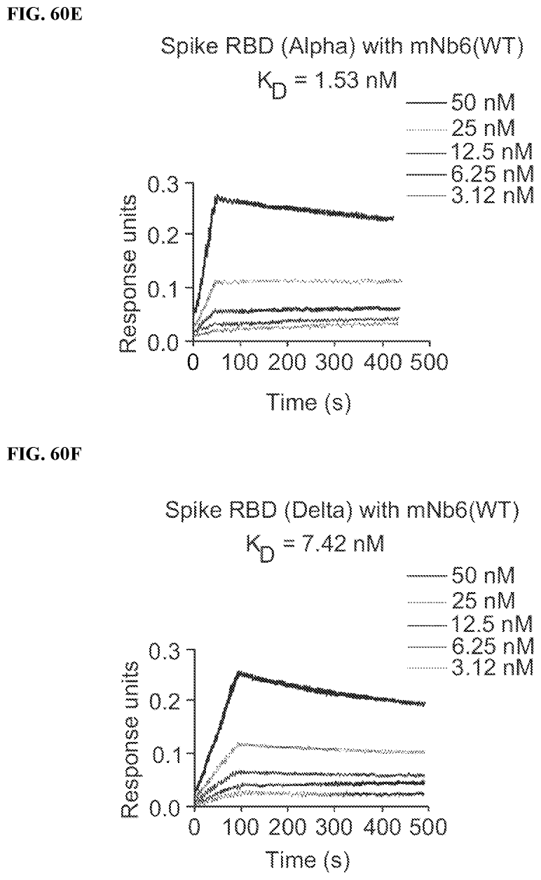

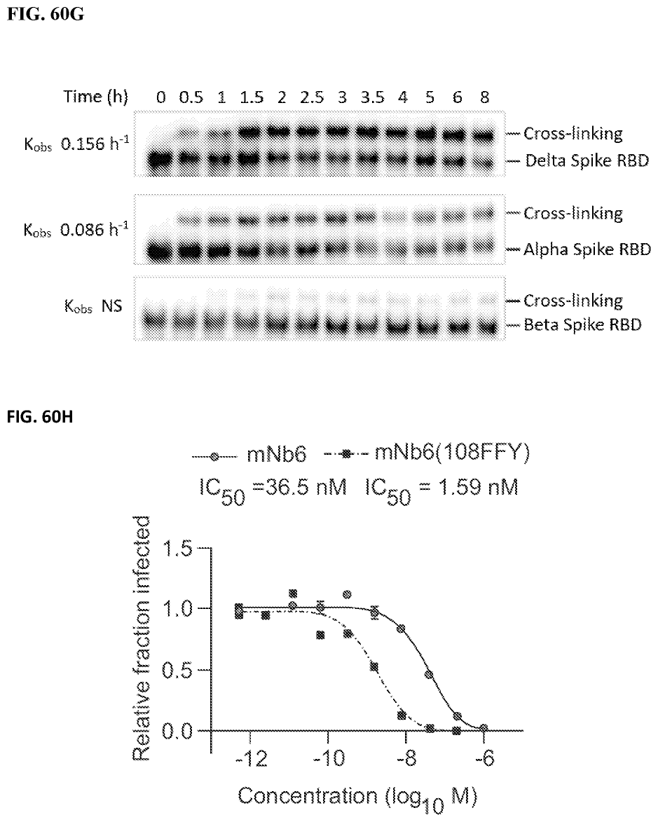

With FFY fast kinetics and mNb6 binding of the down state of Spike RBD, we then tested the efficacy of mNb6(108FFY) in neutralizing SARS-CoV-2 pseudotyped lentivirus infection. Pseudovirus was incubated with varying concentrations of mNb6(108FFY) or mNb6(WT) for 1 h at 37° C., and then the nanobody and pseudovirus mixture was used to infect 293T-ACE2 cells for 48 h at 37° C. The percentage of cell infection was determined by measuring GFP signal via flow cytometry. The IC₅₀ of mNb6(WT) was measured to be 35.7 nM. In contrast, mNb6(108FFY) showed an IC₅₀ of 1.0 nM, exhibiting a marked 36-fold improvement in potency (FIG. 60A). To check if FFY incorporation impacted the binding affinity of mNb6, we measured the binding affinity of Spike RBD with mNb6(WT) or mNb6(108FFY) using biolayer interferometry (BLI). The K_{D} was measured to be 0.99 nM for Spike RBD with mNb6(WT) and 0.99 nM for Spike RBD with mNb6(108FFY) (FIGS. 60B-60C), indicating that FFY incorporation did not significantly affect mNb6 binding affinity. Therefore, the drastically elevated potency of mNb6(108FFY) over mNb6(WT) in neutralizing pseudovirus infection should be attributed not to affinity difference with the Spike RBD but to the effect of covalent binding.

Encouraged by the highly potent effect on SARS-CoV-2 pseudovirus, we further tested the neutralizing efficacy of mNb6(108FFY) on authentic SARS-CoV-2 virus. SARS-CoV-2 Nanoluciferase virus was incubated with nanobodies at 37° C. for 1 h, and the virus-nanobody mixtures were then added to 293T-ACE2 cells for 2 h to allow infection. At 72 h post-infection, luciferase activities of cells were measured to determine the percentage of infection. As shown in FIG. 60D, the IC₅₀ of mNb6(WT) in inhibiting authentic SARS-CoV-2 virus was 69.9 nM; to our delight, the IC₅₀ measured for mNb6(108FFY) was 1.7 nM, indicating a drastic 41-fold improvement in potency.

mNb6(108FFY) Covalently Binds and Potently Inhibits Certain SARS-Cov-2 Variants

Various mutated SARS-CoV-2 strains have emerged during the pandemic. The B.1.1.7 lineage (Alpha), which processes N501Y mutation, shows a stronger interaction with ACE2 and a faster spreading rate; the B.1.351 lineage (Beta), which has K417N, E484K, and N501Y mutations on the RBD, has decreased affinity towards neutralization antibodies and can lower the effectiveness of current vaccine. Delta variant (B.1.617.2), which has L452R and T478K mutation on the RBD, became a dominant strain in many countries. Delta variant has increased transmissibility and is less sensitive to monoclonal antibodies and neutralizing antibodies from recovered individuals, as well as vaccine-elicited antibodies.

We first tested whether these variants would escape from mNb6(WT) binding. BLI measurement showed that mNb6 had a decreased binding affinity towards the RBD of the Alpha and Delta variants. The K_{D} of mNb6(WT) with the Alpha and Delta RBD was 1.24 nM and 7.42 nM, respectively (FIGS. 60E-60F), higher than 0.97 nM, the K_{D} of mNb6(WT) with the WT RBD. In contrast, mNb6(WT) failed to show significant binding with the Beta RBD up to a concentration of 50 nM. We next determined whether mNB6(108FFY) could crosslink the RBD of various variants. Western blot analysis indicated that mNB6(108FFY) efficiently cross-linked with the Delta and Alpha RBD (FIG. 60G), although the cross-linking rate (k_{obs}=0.156±0.036 h⁻¹ for Delta and 0.086±0.008 h⁻¹ for Alpha) was slightly slower than that with the WT RBD (k_{obs}=0.244±0.031 h⁻¹), consistent with the decreased affinity towards these variants. For the Beta RBD, although the affinity was dramatically reduced, mNB6(108FFY) was still able to crosslink it albeit in low efficiency (about 5%) (FIG. 60G). These results demonstrate that the covalent mNb6(108FFY) was capable of cross-linking the Spike RBD of the SARS-Cov-2 variants.

We further assessed the efficacy of mNb6(108FFY) in neutralizing pseudovirus of SARS-CoV-2 variants. From the inhibition curves of pseudovirus infection, the IC₅₀ of mNb6(WT) in inhibiting the Alpha variant pseudovirus was 36.5 nM (FIG. 60H); in contrast, mNb6(108FFY) had an IC₅₀ of 1.6 nM, showing a 23-fold increase in potency. For the quickly spreading Delta variant, mNb6(WT) inhibited the pseudovirus with an IC₅₀ of 143 nM (FIG. 60I); gratifyingly, mNb6(108FFY) was able to neutralize the Delta variant pseudovirus with an IC₅₀ of 3.7 nM, achieving a drastic 39-fold improvement in potency. In comparison with the WT SARS-Cov-2 pseudovirus, mNb6(WT) inhibited the Delta variant pseudovirus with a much higher IC₅₀ (143 nM vs 35.7 nM), which is consistent with the decreased affinity of mNb6(WT) towards the Delta RBD than the WT one (7.42 nM vs 0.97 nM). However, despite the marked decrease in binding affinity of mNb6(WT) towards Delta RBD, mNB6(108FFY) still inhibited the Delta variant pseudovirus effectively when compared with the WT pseudovirus (IC₅₀ of 3.7 nM vs 1.0 nM). In short, the covalent mNb6(108FFY) nanobody also potently inhibited the Alpha and Delta variant viruses, with respective 23- and 39-fold increase in potency than the mNb6(WT) nanobody.

Covalent mNb6 Dimer Further Enhances Viral Neutralization Over Noncovalent WT Dimer

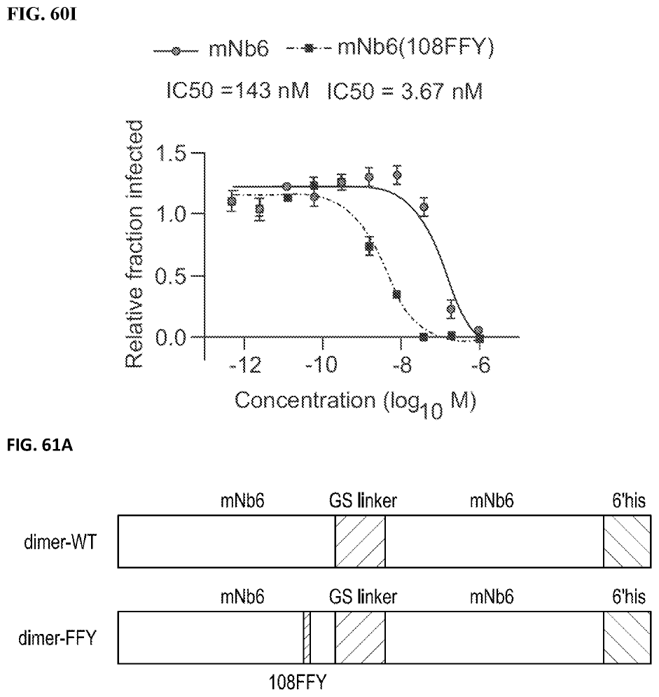

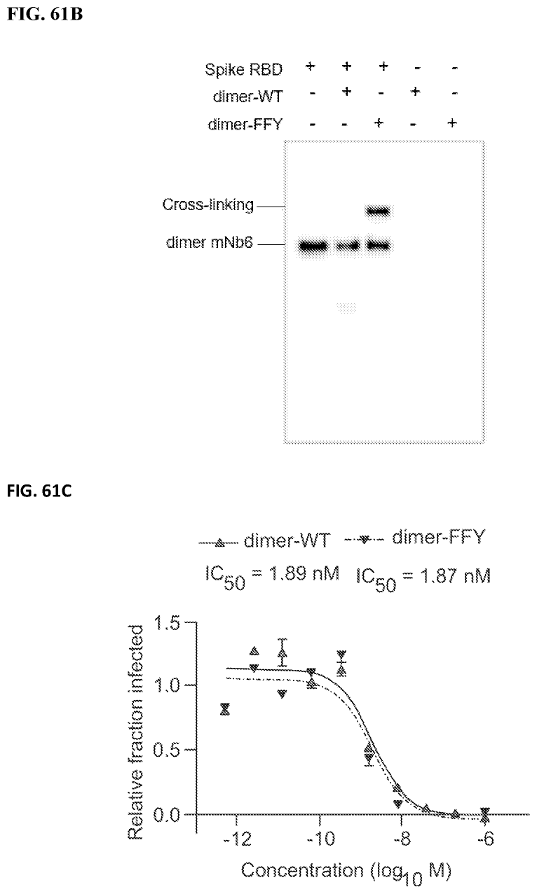

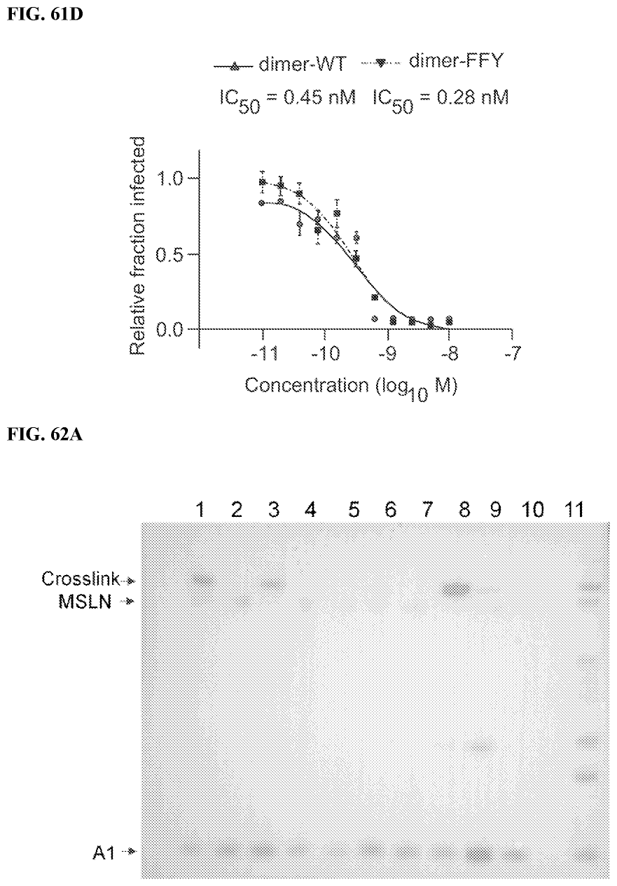

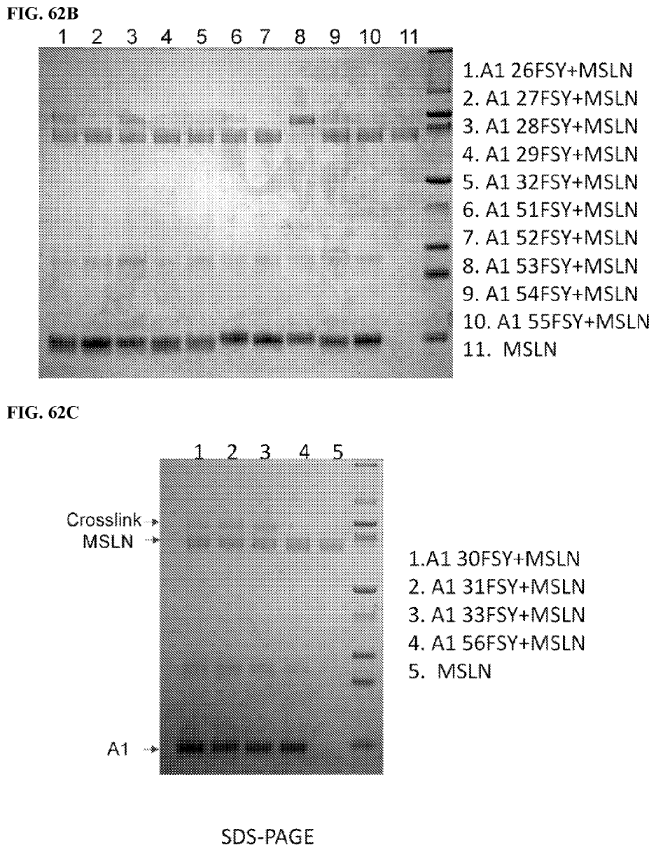

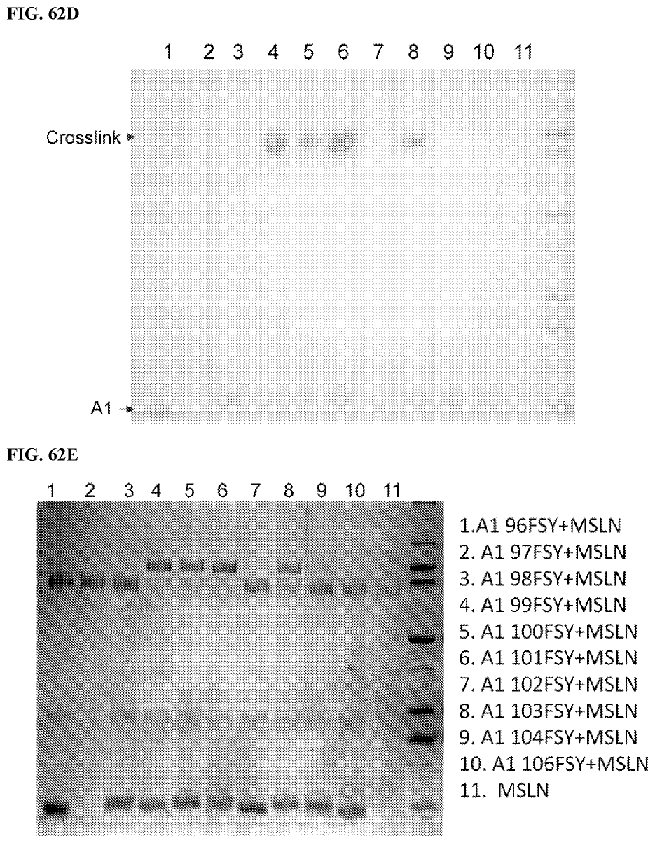

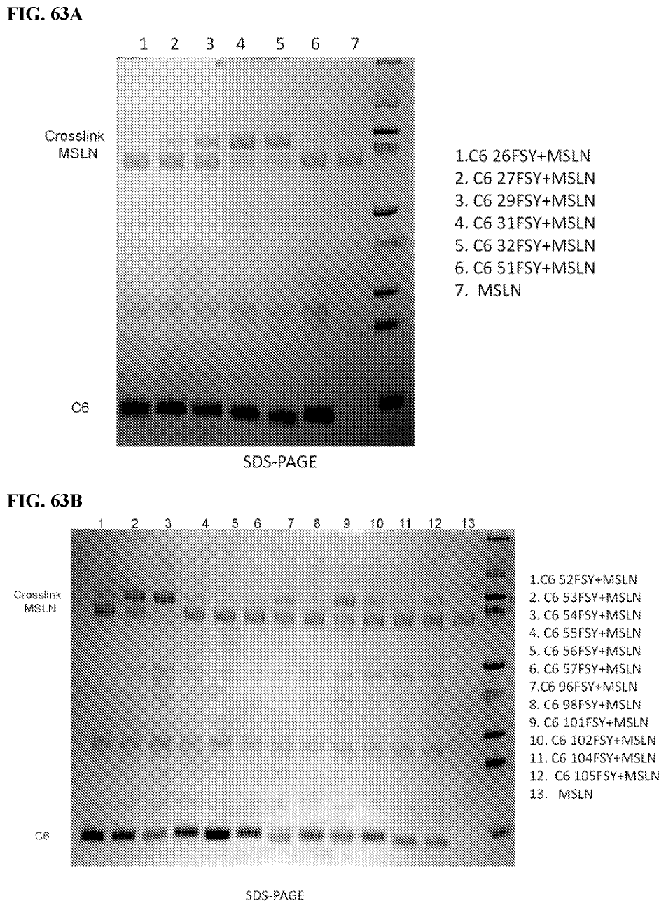

Dimerization of nanobody has been proved to be an effective strategy to improve the potency. We are motivated to know if incorporation of FFY into mNb6 monomer would elevate the potency of this monomer to the level of its dimer form. We then constructed the dimer form of mNb6. Two wildtype mNb6 monomer was linked by a glycine-serine (GS) linker to form wild-type mNb6 dimer, which was referred as dimer-WT for clarity. The IC₅₀ of dimer-WT in neutralizing SARS-CoV-2 pseudotyped lentivirus infection is 1.9 nM, which is similar with mNb6(108FFY) in the monomer form but is 28-fold more potent than mNb6(WT). To test if the potency of mNb6 dimer can be further improved by converting it to the covalent form, we inserted one FFY at position 108 in the N-terminal of mNb6 dimer and this mutated dimer was referred as dimer-FFY (FIG. 61A). We first tested if dimer-FFY mutants could bind to RBD covalently in vitro. We incubated 2 μM Spike RBD-mFc with 2 μM dimer-WT or dimer-FFY at 37° C. for 12 h, followed by Western blot analysis against mFc. dimer-WT did not form a covalent complex with the Spike RBD, whereas a stable covalent complex was detected between dimer-FFY and the Spike RBD (FIG. 61B). The IC₅₀ of dimer-FFY in in neutralizing pseudovirus was determined to be 1.9 nM, the same as dimer-WT (FIG. 61C). However, the IC₅₀ of dimer-FFY in in neutralizing authentic SARS-CoV-2 virus was determined to be 0.28 nM, while for dimer-WT, the IC₅₀ is 0.45 nM, showing a moderate potency improvement of dimer-FFY over dimer-WT (FIG. 61D).

We also introduced F-FSY into the maltose binding protein (MBP) fused Z protein at position 24 (MBP-Z(24F-FSY)). The purified MBP-Z(24F-FSY) was analyzed by electrospray ionization time-of-flight mass spectrometry (ESI-TOF MS). A peak observed at 50829 Da corresponds to intact MBP-Z containing F-FSY at site 24 (data not shown). We then tested the reactivities of F-FSY towards amino acid residue using the binding complex of MBP-Z and the Zspa affibody (Afb). Upon Afb-Z binding, F-FSY would be place in close proximity with the residue at position 7 of Afb (Afb7X) and would form a covalent bond with the targeting residue, causing protein-protein cross-linking. As shown in FIG. 19E, F-FSY would form a covalent bond with Lys, His and Tyr, the same as FSY.

### Methods

### Nanobody Expression and Purification

Plasmid pBAD-H11D4, pBAD-MR17K99Y, or pBAD-SR was transformed into E. coli BL21(DE3) for wildtype protein expression. Plasmid pBAD-H11D4 (27TAG), pBAD-H11D4 (30TAG), pBAD-H11D4 (100TAG), pBAD-H11D4 (112TAG), pBAD-H11D4 (115TAG), pBAD-H11D4 (116TAG), pBAD-MR17K99Y (99TAG), pBAD-MR17K99Y (101TAG), pBAD-SR4 (37TAG), pBAD-SR4(54TAG), or pBAD-SR4 (57TAG) was co-transformed respectively with plasmid pEVOL-FSYRS into E. coli BL21(DE3) for FSY incorporated protein expression. Transformed E. coli cells were grown at 37° C., 220 rpm to an OD 0.5, after which 0.2% L-arabinose was added. For FSY incorporation, 1 mM FSY was added to the growth medium. The expression was carried out at 18° C., 220 rpm for 18 h. Cells were harvested at 8000 g, 4° C. for 30 min. The cell pellet was suspended with lysis buffer (20 mM Tris-HCl, 20 mM imidazole, 200 mM NaCl, pH 7.5) supplemented with EDTA free protease inhibitor cocktail and 1 μg/mL DNase. The cells were lysed by sonification, after which the cell lysis solution was centrifuged at 25,000 g at 4° C. for 40 min. The supernatant was collected and purified with 500 μL Ni-NTA resin affinity resin. The resin was washed and eluted with elution buffer (20 mM Tris-HCl, 300 mM imidazole, 200 mM NaCl, pH 7.5). The eluted protein was concentrated and exchanged with buffer (20 mM Tris-HCl, 200 mM NaCl, pH 7.5). To yield pure protein, TALON® metal affinity resin was further applied. The purification procedure was same as described above. The eluted proteins were analyzed by running 12% Tris-glycine SDS-PAGE gel.

Cross-Linking Study of Nanobody with Spike RBD

The Spike RBD was incubated with wildtype nanobody or nanobody mutants at indicated concentrations in PBS (pH 7.4) at 37° C. for 12 h, after which 5 μL sample was mixed with 10 μL Laemmli loading dye supplied with 100 mM DTT. The mixture was boiled at 95° C. for 10 min and subjected to Western blot analysis. The bands were detected using an antibody against mouse Fc appended at the C-terminus of the Spike RBD.

Cross-Linking Kinetic Study of Spike RBD with mNB6(108FFY) or mNB6(108FSY)

The SARS-CoV-2 WT or variant Spike RBD (His×6 tagged, 0.5 μM) was incubated with 5 μM mNB6(108FSY) or mNB6(108FFY) in PBS (pH 7.4) at 37° C. At different time points, 5 μL reaction mixture was extracted and mixed with 5 μL Laemmli loading buffer. The mixture was heated to 95° C. for 10 min and the protein cross-linking was examined by Western blot. The protein band was detected with HRP-conjugated anti-His×6 antibody (Proteintech, #HRP-66005). The Spike RBD band intensity in the Western blot was quantified with Bio-rad imaging software. The linear plot of natural logarithm (ln) of the Spike RBD band intensity versus time (h) gives k_{obs}.

### Binding Constant (K_{D}) Measurement Between Spike RBD and Nanobody

Binding constant (K_{D}) between Spike protein RBD and nanobody was measured with biolayer interferometry (BLI) using Octet Red384 systems (ForteBio). Biotinylated Spike RBD was firstly loaded to streptavidin (SA) sensor (ForteBio #18-5019) by incubating SA sensor in 100 nM biotinylated Spike RBD in Kinetic Buffer (0.005% (v/v) Tween 20 and 0.1% BSA in PBS, pH=7.4) at 25° C. The sensor was equilibrated (baseline step) in Kinetic Buffer for 120 s, after which the sensor was incubated with varying concentrations of nanobody (association step) for 50 s, followed with dissociation step in Kinetic Buffer for 450 s. Data was fitted for a 1:1 stoichiometry and K_{D} was calculated using the built in software.

### SARS-CoV-2 Pseudovirus Assay for Nanobody Neutralization

The SARS-CoV-2 GFP Reporter Virus Particles (RVPs) are SARS-CoV-2 pseudotyped lentivirus and were purchased from Integral Molecular. Catalog numbers for strains are the following: wild-type strain: RVP-701; Alpha: RVP-706; Beta: RVP-714; Delta: RVP-763. One day before transduction, 4×10⁴ 293T-ACE2 cells were plated in each well of a 48-well plate. Serially diluted nanobodies were incubated with pseudovirus in DMEM at 37° C. for 1 h. The mixture was subsequently transferred to each well of the 48-well plate. The cells were cultured at 37° C. for additional 48 h, after which the cells were harvested for flow cytometric analysis to measure the proportion of GFP positive cells.

### Authentic SARS-CoV-2 Neutralization Assay

SARS-CoV-2 Nanoluciferase (USA/WA1-2020) (SARS-CoV2 nLuc) was a kind gift from Dr. Pei-Yong Shi. The virus stocks were prepared in Vero E6 (ATCC) and titers were determined by plaque assays on Vero-E6 cells. Neutralizing assays were performed in 293T-ACE2 cells (15,000 cells per well) in a white opaque 96-well plates. Input virus (multiplicity of infection 0.01) was mixed with media containing nanobodies at indicated final concentrations and incubated for 1 h at 37° C. The virus-nanobody mixtures were then added to cells for 2 h to allow virus adsorption, and washed. At 72 h post-infection, NanoLuc Luciferase substrates (Promega) was added to each well and luciferase signals were measured using a GloMax microplate reader (Promega). The relative luciferase signals were normalized to no-nanobody control. Virus propagation and experiments were performed in the BSL3 facility.

Primers were synthesized and purified by Integrated DNA Technologies (IDT), and plasmids were sequenced by GENEWIZ. All molecular biology reagents were either obtained from New England Biolabs or Vazyme. His-HRP antibody was obtained from ProteinTech Group. SARS-CoV-2 spike protein, RBD, 6His tag (S protein) was purchased from ACRObiosystems (#SRD-C52H3). All solvents were of reagent grade and were purchased from Fisher Scientific and Aldrich. Reagents were purchased from Aldrich and Asta Tech. The stationary phase of chromatographic purification is silica (230×400 mesh, Sorbtech). Silica gel TLC plate was purchased from Sorbtech. ¹H-NMR (400 MHz) and ¹³C-NMR (100 MHz) spectra were recorded on a Bruker Avance 400 MHz NMR spectrometer. OD600 and fluorescence intensity were recorded on BioTek UV/Vis/Fluorescence plate reader.

Incorporation of F-FSY into Protein

EGFP (182TAG) and MBP-Z(24TAG) were cloned into the expression plasmid pBAD as reported in Wang et al, J. Am. Chem. Soc., 140:4995-4999 (2018). pBAD-EGFP (182TAG), pBAD-MBP-Z(24TAG) or pBAD-mNB6-108 (TAG) was co-transformed with pEVOL-FSYRS into DH10b, and plated on LB argar plate supplemented with 50 j.tg/mL ampicillin and 34 j.tg/mL chloramphenicol. A single colony was picked and inoculated into 1 mL 2×YT (5 g/L NaCl, 16 g/L Tryptone, 10 g/L Yeast extract) with 50 j.tg/mL ampicillin and 34 j.tg/mL chloramphenicol. The cells were left grown 37° C., 220 rpm for overnight. Next morning, the cells were diluted 100 times in fresh 2×YT supplemented with 50 j.tg/mL ampicillin and 34 j.tg/mL chloramphenicol. When cells reach an OD600 of 0.5, cells were supplied with 2 mM F-FSY. The cells were then induced by 0.2% arabinose either at 18° C. for 20 h for MBP-Z and EGFR or 25° C. for 20 h for mNB6. Proteins were then purified following the procedure.

### His-Tagged Protein Expression and Purification

Afb7X was were cloned into the expression plasmid pBAD and expressed in E. coli following Wang et al, J. Am. Chem. Soc., 140:4995-4999 (2018). After protein expression, 100 mL cells were centrifuged at 4,000 rpm for 10 min and the cell pellet was suspended in cell lysis buffer (50 mM Tris-HCl pH 8.0, 500 mM NaCl, 20 mM imidazole, 1% v/v Tween20, 10% v/v glycerol, DNase 0.1 mg/mL) with protease inhibitors. Lysate was sonicated with Sonic Dismembrator (Fisher Scientific, 30% output, 5 min, 3 s off, 3 s on) in an ice-water bath, after which the lysate was centrifugated (4,000 rpm for 10 min) and the supernatant was collected. Ni-NTA Agarose slurry (Thermo Scientific, #88222, 200 j.tL) was rinsed with wash buffer (50 mM Tris-HCl pH 8.0, 500 mM NaCl, 20 mM imidazole, 10% v/v glycerol) and added to the supernatant. The mixture was incubated at 4° C. for 15 min and subsequently loaded onto a Poly-Prep® Chromatography Column. After washing the column 3 times with 20 mL PBS (pH 7.4) containing 20 mM imidazole, 1 mL elution buffer (PBS with 250 mM imidazole) was used to elute the protein. Purified protein was exchanged to PBS (pH 7.4) using Amicon Ultra column and stored at −20° C.

### Afb7X and MBP-Z(24F-FSY) Cross-Linking

0.5 mg/ml Afb7X and 1 mg/ml MBP-Z(24F-FSY) were incubated in PBS (pH 7.4) at 37° C. for 12 h, after which 1 j.tL reaction solution was extracted and mixed with 10 j.tL Laemmli loading buffer. The mixture was heated to 95° C. for 10 min and then loaded for SDS-PAGE, after which the gel was stained with Coomassie blue and imaged with ChemiDoc™ MP imaging system (Bio-rad).

S Protein and mNB6(108F-FSY) Cross-Linking

0.5 j.tM S protein (6His tag) was incubated with 5 j.tM mNB6(108FSY) or mNB6(108F-FSY) in PBS (pH 7.4) at 37° C. At different time points, 1 j.tL reaction mixture was extracted and mixed with 5 j.tL Laemmli loading buffer. The mixture was heated to 95° C. for 10 min and the protein cross-linking was exanimated by western-blot. The protein band was detected with HRP-conjugated anti-6His antibody (Proteintech, #HRP-66005).

Kobs was calculated based on the decrease intensity of S protein band. The spike protein band intensity was quantified by Bio-rad imagine software based on western-blot. The linear plot of natural logarithm (ln) of the spike protein intensity versus time (h) gives Kobs.

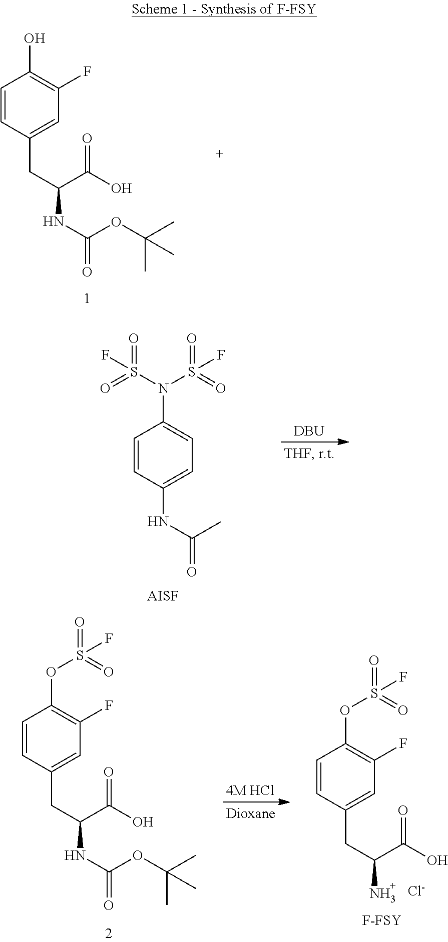

Synthesis of compound 2. Compound 1 was converted to fluorosulfate using [4-(acetylamino)phenyl]imidodisulfuryl difluoride (AISF) (Zhou et al, Fluorides Org. Lett, 20:812-815 (2018). 1.0 g compound 1 (3.3 mmol) and AISF (1.3 g, 4.0 mmol) was dissolved in 12 ml anhydrous THF. Then 1,8-Diazabicyclo[5.4.0]undec-7-ene (DBU, 1.1 g, 7.3 mmol) was added dropwise at room temperature (r.t.). The mixture was stirred at r.t. for 10 min. Then 100 ml EtOAc was added to dilute the reaction mixture and the organic phrase was washed sequentially by H2O (50 mL) and brine (50 mL). The organic phase was dried over anhydrous Na2SO4 and evaporated under reduced pressure to give the crude product, which was then purified by column chromatography (silica gel, DCM:MeOH=50:1) to give a white solid (0.8 g, 64%).

Synthesis of F-FSY. Compound 2 (0.8g, 2.1 mmol) was stirred in 4 M HCl in dioxane (10 ml) at r.t. for 6 h. Then 10 ml diethyl ether was added to the reaction mixture, and a white precipitate was formed and collected by filtration. The white solid was further dried under reduced pressure to give F-FSY in HCl salt form (604 mg, 90%). ¹H NMR (D2O): δ 7.63-7.59 (m, 1H), 7.39 (dd, J=10.8 Hz, J=2.0 Hz, 1H), 7.29-7.26 (m, 1H), 4.27-4.24 (m, 1H), 3.41-3.24 (m, 2H). 13C NMR (D2O): δ 172.2, 153.8 (d, J=252 Hz, C—F), 138.4, 136.8 (d, J=13 Hz, C—F), 127.1 (d, J=3 Hz, C—F), 124.4, 119.4 (d, J=18 Hz, C—F), 54.8, 35.8; HRMS calcd for C9H10F2NO5S [M+H]⁺ 282.0242, found 282.0253.

### Example 4

Currently, using nanobody or single chain antibody for molecular imaging has been challenging. To address this challenge, we genetically-encoded a covalent fluorosulfate unnatural amino acid and through proximity-enabled reactivity, covalent crosslink the nanobody to its target (CoNPET). We demonstrated through positron emission tomography (PET) imaging that the nanobody remained accumulated at the target tumor site, having very high tumor-to-muscle and tumor-to-blood ratio.

Immuno-positron emission tomography (immunoPET) uses monoclonal antibodies labelled with a positron emitter to achieve tumor imaging with high specificity. (Refs 1, 2). However, due to its long persistence in blood circulation, whole antibodies are not ideal for PET imaging. (Refs 3, 4). Genetically engineered nanobodies, affibodies, or antibody fragments used for molecular imaging have the advantage of quick tumor penetrance and shorten retention time compared to whole antibodies. (Refs 5-7). While these small protein binders have a short retention time, they also suffer from short target accumulation and often do not have good contrast compared to normal tissues. (Refs 8, 9). An ability to increase the tumor accumulation while having quick blood clearance would be the most ideal PET imaging tool for tumor imaging.

There has been a recent resurgence of covalent probes due to their increased potency and efficacy, improved therapeutic index, on-target residence time, and enhanced selectivity.¹⁰ These covalent probes have specifically been applied to PET imaging particularly for their increased in vivo on-target activity and selectivity. (Ref 11). However, until recently, covalent targeting has largely been subjected to small molecule drugs due to the lack of covalent chemistries involved with proteins. Several studies reported genetically encoding a latent bioreactive unnatural amino acid (Uaa) into proteins to form covalent bond between the protein binder and its target. (Refs 12-14) These bioreactive Uaas are chemically stable in cells and only react when a nucleophilic amino acid comes in close proximity. By forming a covalent linkage between the target and the protein binder, the protein binder increases its efficacy and on-target residence time. This technology has been successfully applied to covalent protein drugs showing increased efficacy and minimizing off-targets. (Ref 15).

In this study, we developed a covalent nanobody PET probe (CoNPET), a nanobody PET probe with a genetically encoded bioreactive Uaa, that covalently links the nanobody to its target. We applied this technology to target human epidermal growth factor receptor 2 (HER2) for nuclear imaging. HER2 gene amplification and overexpression occurs in a number of different cancers including breast, stomach, ovarian, kidney, prostate, salivary glands, colon, urinary, and lung. Particularly, about 20% of breast cancers have HER2 overexpression. (Ref 16). To image HER2-positive cancer, PET has been the modality off choice for the clinic due to its high spatial resolution and sensitivity. (Ref 17). There have been several successful clinical antibodies, such as trastuzumab (Herceptin) that have been developed to target HER2-positive cancers. (Ref 16). While, these antibodies have shown considerable efficacy towards HER2-positive cancers, they have been limited in molecular imaging due to their large size leading to slow clearance and low tumor penetrance. (Refs 3, 4). These antibodies are also incompatible with short-lived radionuclides, since it often takes several days to attain reasonable imaging contrast. For these reasons, alternative small protein (less than 30 kDa) binders have been investigated for molecular imaging including affibodies, single chain antibodies, and nanobodies. (Ref 17). While the blood and tissue clearance increased dramatically over clinical antibodies, the small protein accumulated quickly in the kidney and liver causing high background activity in these organs and low tumor accumulation. To address this problem, we genetically-encoded a bioreactive unnatural amino acid into a nanobody to covalently target HER2 to increase its on-target residence time and allow for high contrast on the tumor.

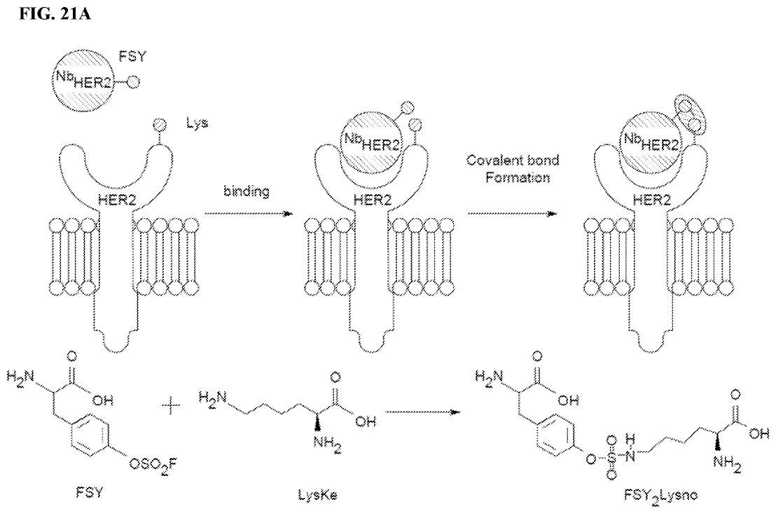

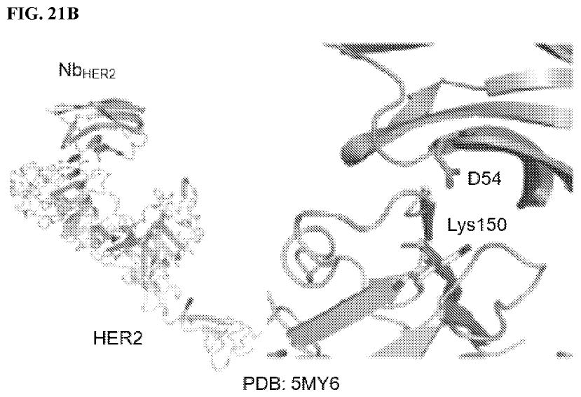

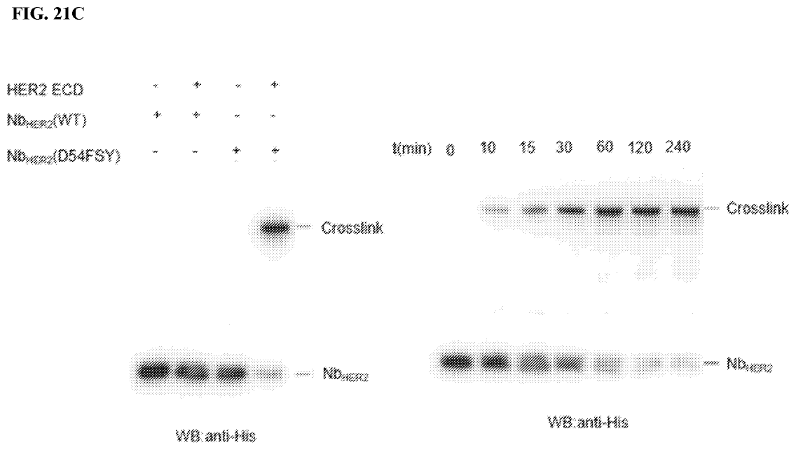

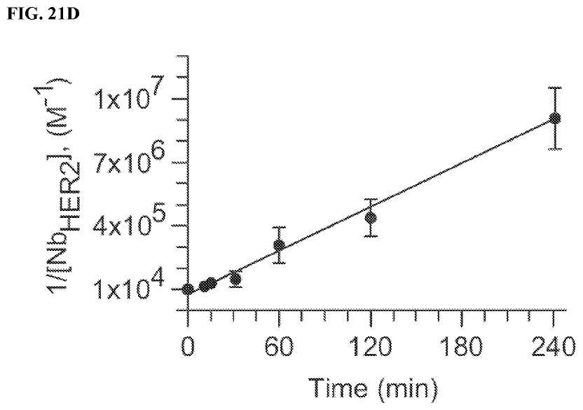

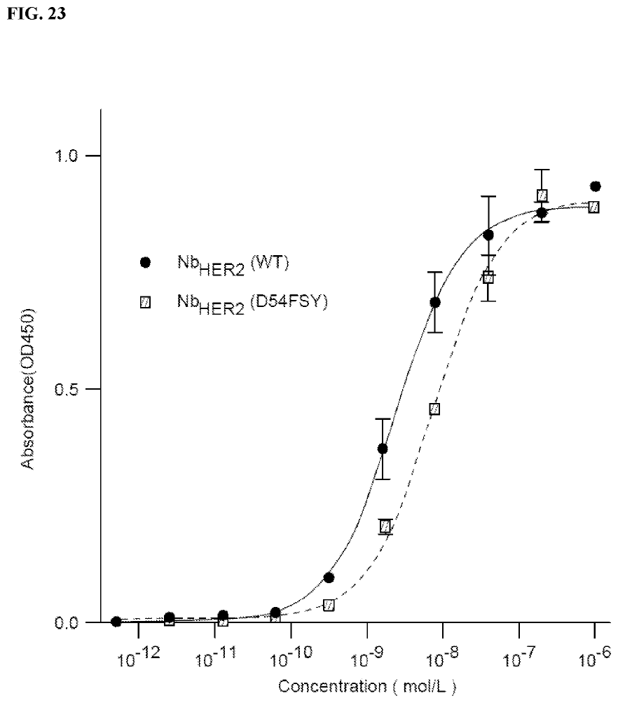

We previously developed several bioreactive unnatural amino acid that target different nucleophilic amino acids on the target protein. Fluorosulfate-containing unnatural amino acid has been particularly successful due to its ability to target lysine, histidine and tyrosine only when it comes in close proximity. (Refs 18, 19). Here, we incorporated fluorosulfate-L-tyrosine (FSY) Uaa into the nanobody (NbHER2, also referred to as 2rs15d, set forth as SEQ ID NO:66) binding site in proximity to a lysine residue on the HER2 extracellular domain (ECD) (FIG. 21A). The FSY Uaa undergoes a sulfur-fluoride exchange (SuFEx) click reaction forming and irreversible covalent bond with the lysine. Based on the structure of the nanobody bound to the HER2 ECD, we identify D54 (Nb_{HER2} (D54FSY)) on the nanobody as a potential site to target K150 on HER2 ECD (FIG. 21B). (Ref 20). The purified Nb_{HER2} (WT) (SEQ ID NO:66) and Nb_{HER2} (D54FSY) (SEQ ID NO:71) was characterized through ESI-TOF Mass spectrometry showing the increase in mass with the FSY-incorporated nanobody over the WT. To test if Nb_{HER2} (D54FSY) can form a covalent complex with the HER2 ECD, we incubated Nb_{HER2}(WT) and Nb_{HER2} (D54FSY) at 37° C. over 4 h, with and without HER2 ECD and analyzed the interaction through western blot. We observed crosslinking when HER2 ECD and Nb_{HER2} (D54FSY) were incubated together indicating the covalent complexed formed (FIG. 21C). We next tested if the covalent complex can form rapidly and how fast the reaction occurs. Again, through western blot, the covalent complexed formed within 10 min of incubation at 37° C. with a second-order rate constant of 34154±1921 M⁻¹ min⁻¹ (FIG. 21D). To confirm that the FSY incorporation did not significantly lower the binding affinity of the nanobody, we performed an enzyme linked immunosorbent assay (ELISA) (FIG. 23). The affinity only dropped around 3-fold, but the Nb_{HER2} (D54FSY) still had nanomolar affinity (7.60 nM).

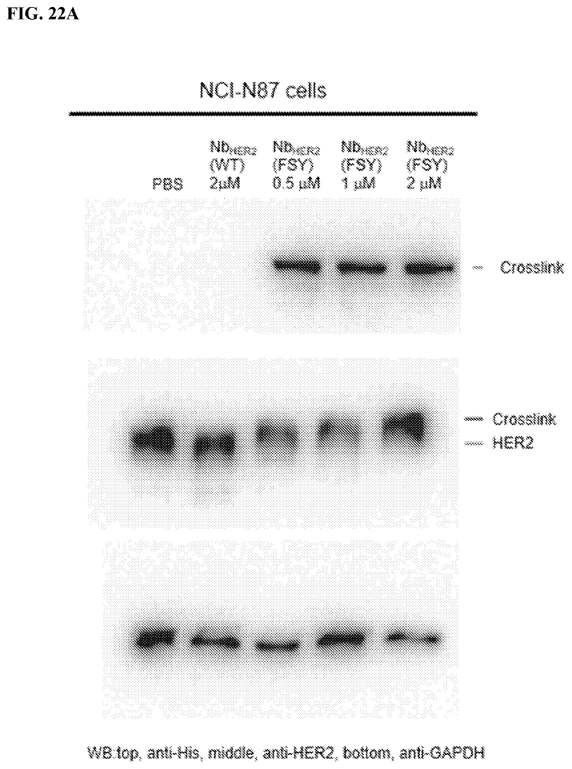

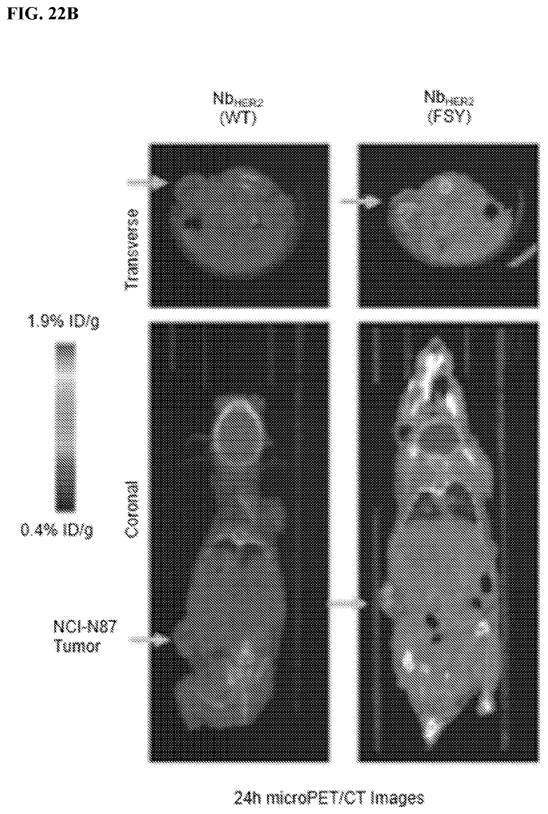

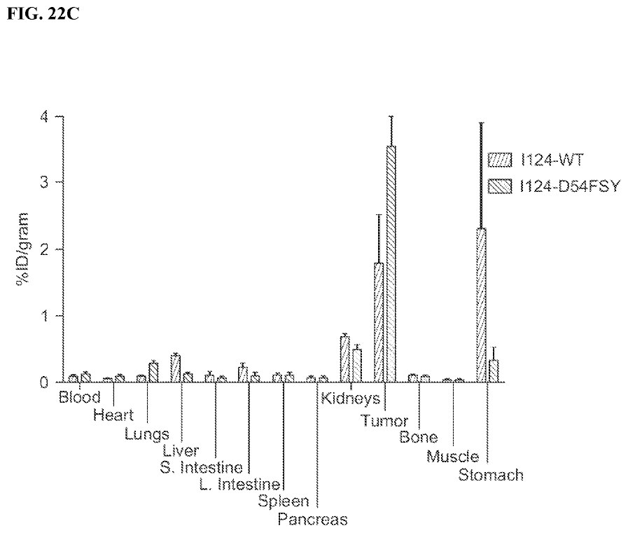

We next tested if Nb_{HER2} (D54FSY) could covalently crosslink HER2 on the cell surface of NCI-N87, a HER2-positive gastric cancer. We treated cultured NCI-N87 with a different concentration of Nb_{HER2} (D54FSY) and compared to the PBS and Nb_{HER2} (WT). We then washed the cells to remove any non-covalently bound binders and analyzed the covalent complexes through western blot. PBS and Nb_{HER2} (WT) did not show any crosslinking complexes, whereas Nb_{HER2} (D54FSY) showed crosslinking at all concentrations. (FIG. 22A). Taken together, both the in vitro crosslinking assay and on-cell crosslinking gave robust formation of the Nb-HER2 covalent complex.

To examine if our Nb_{HER2} (D54FSY) could give enhanced accumulation and high tumor-to-blood contrast we compared radiolabeled Nb_{HER2} (WT) and Nb_{HER2} (D54FSY) and observed the radioligand through microPET/CT imaging. First, Nb_{HER2} (WT) and Nb_{HER2} (D54FSY) were labelled with ¹²⁴Iodine, a positron emitter suitable for PET. Male nude mice bearing subcutaneous NCI-N87 were then injected with ¹²⁴I-Nb_{HER2} (WT) or ¹²⁴I-Nb_{HER2} (D54FSY). The mice were then imaged 24 h post-injection and the biodistribution was conducted to evaluate the radiotracer distribution in normal tissues. The on-tumor activity for ¹²⁴I-Nb_{HER2} (D54FSY) compared to ¹²⁴I-Nb_{HER2} (WT) is drastically different with ¹²⁴I-Nb_{HER2} (D54FSY) showing much higher activity on the tumor (FIG. 22B). The ex vivo biodistribution analysis shows a 2-fold increase in activity on the tumor (FIG. 22C). Once ¹²⁴I dissociates from the nanobody, it accumulated in the thyroid therefore high activity is observed there as well. Background activity for most normal tissues was low for both ¹²⁴I-Nb_{HER2} (WT) or ¹²⁴I-Nb_{HER2} (D54FSY).

Overall, we report CoNPET, a covalent nanobody technology useful for molecular imaging that enables us to give high contrast tumor detection and low background activity in all other tissues. To this date, ImmunoPET relies on large antibodies that has low blood clearance and may take days to image due to low and slow tumor penetrance. (Refs 3, 4). Nanobodies and other small protein binders provide an alternative to achieve better pharmacokinetics for faster imaging. Unfortunately, the fast clearance also leads to short target retention time and low contrast on tumor activity. (Refs 5-7). CoNPET takes advantage of the fast clearance to nanobodies but uses covalent crosslinking to have much higher accumulation on the tumor target. CoNPET represents a new class of radioligands that can be valuable to molecular imaging as well as radionuclide therapy.

### Example 5

Processes for producing fluorosulfate-L-tyrosine; incorporating fluorosulfate-L-tyrosine into proteins; and forming complexes of covalently bonded proteins are described in WO 2020/206341 and in Wang et al, J. Am Chem Soc, 140(15):4995-4999 (2018) and the Supporting Information associated with this publication.

### Example 6

Using a soluble ACE2 receptor binding to the viral S protein thereby neutralizing SARS-CoV-2 is an attractive strategy. First, the S protein of SARS-CoV-2 binds to the ACE2 receptor with a K_{D} of 4.7 nM, comparable to affinities of mAbs. Lan et al, Nature, 1-9 (2020). Second, ACE2 administration could additionally treat pneumonia caused by SARS-CoV-2. Coronavirus binding leads to ACE2 protein shedding and downregulation, which induces pulmonary edema and acute respiratory distress syndrome (ARDS). Administration of recombinant human ACE2 (rhACE2) improves acute lung injury and reduces ARDS in preclinical studies. Imai et al, Nature, 436(7047):112-116 (2005); Khan et al, Crit Care, 27(1):234 (2017). Third, rhACE2 is safe and well tolerated by healthy volunteers in phase I trial and by patients in phase II trial, and small levels of soluble ACE2 are secreted and circulate in human body. Haschke et al, Clin Pharmacokinet 52(9):783-792 (2013); Khan et al, Cri Care, 27(1):234 (2017); Shao et al, J. Card. Fail., 19(9):605-610 (2013). Fourth, fusion of an Fc domain to the soluble ACE2 could extend its half-life from 2 h to one week in mice (13), and the Fc effector functions could recruit immune cells against viral particles or infected cells. Liu et al, Kidney Int., 94(1):114-125 (2018). Fifth, the soluble ACE2 therapy is expected to have broad coverage, because SARS-CoV-2 cannot escape neutralization due to its dependence on the same protein for cell entry. Any mutation of SARS-CoV-2 reducing its affinity for the drug will render the virus less pathogenic.

rhACE2 was reported to reduce SARS-CoV-2 recovery from Vero cells and to inhibit SARS-CoV-2 infection of human blood vessel and kidney organoids. Monteil et al, “Inhibition of SARS-CoV-2 Infections in Engineered Human Tissues Using Clinical-Grade Soluble Human ACE2,” Cell, (2020). However, the inhibition was far from complete, and inhibition was observed in kidney organoids only when high concentration (200 μg/mL) of rhACE2 was applied. More critically, in all experiments the author preincubated rhACE2 with SARS-CoV-2 virus for 30 minutes, and then applied the mixture to infect Vero cells or organoids. Preincubation of rhACE2 with the virus cannot accurately evaluate whether rhACE2 can inhibit virus in infected patients, where the virus already bind the cell receptor; rhACE2, as a treatment administrated later than virus binding, may not be able to compete the bound virus off cells.

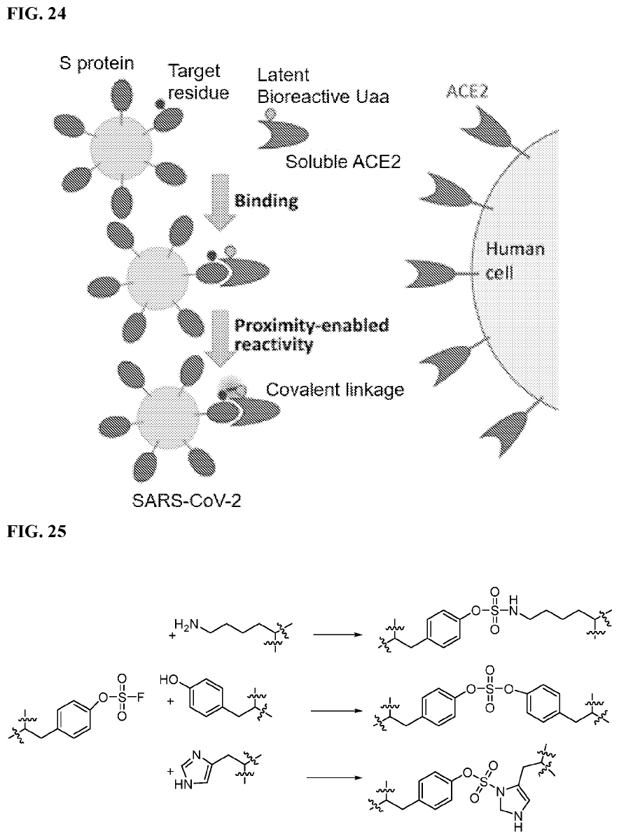

The inventors propose a novel strategy to address this long-lasting challenge: converting the protein inhibitor (which is noncovalent) into a covalent protein inhibitor through a Proximity-Enabled Reactive Therapeutic (PERx) mechanism. Covalent binding is irreversible and has zero off-rate. The covalent protein inhibitor described herein can compete for binding with viral ligands of virus that already bound with cells, because the binding equilibrium will be thermodynamically favoring the covalent protein inhibitor, eventually leading to complete inhibition. In essence, covalent binding affords the covalent protein inhibitor “infinite affinity” for the viral ligand, which is unattainable with conventional protein drugs. To achieve covalent binding, the inventors genetically incorporated into rhACE2 a latent bioreactive unnatural amino acid (Uaa), which remains chemically inert in proteins and in vivo (FIG. 24). Only upon rhACE2-S protein binding, the Uaa specifically reacted with a proximal natural residue of the S protein via proximity-enabled reactivity, allowing rhACE2(Uaa) to covalently bind to the viral S protein selectively. Xiang et al, Nat Methods, 10(9):885-888 (2013). Covalent reactivity in PERx necessitates both drug-target binding and Uaa-natural residue pairing, thus uniquely affording ultimate specificity and target selectivity.

Genetic incorporation of FSY into ACE2 proteins in HEK293T and Expi293 cell. In Wang et al, J Am Chem Soc, 140(15):4995-4999 (2018), the inventors reported the evolution of an orthogonal tRNA^{Pyl}_{CUA}/FSYRS pair for genetic incorporation of fluorosulfate-L-tyrosine (FSY) in both E. coli and mammalian cells. FSY is a latent bioreactive Uaa, which selectively form stable covalent bonds with Tyr, Lys, and His in proximity. By genetically encoding FSY in ACE2, the cellular receptor for severe acute respiratory syndrome-coronavirus (SARS-CoV) and SARS-CoV-2, the resultant FSY-modified ACE2 could act as covalent inhibitors of SARS-CoV and SARS-CoV-2. Based on the co-crystal structure of ACE2/SARS-CoV-2 complex, FSY was incorporated into the soluble human ACE2 at sites D30, H34, E37, D38, Q42, and Y83 to covalently target proximal residues K417, Y453, Y505, Y449, Y449, and Y489 of the S protein, respectively (FIGS. 2, 3A, 3B). Lan et al, “Structure of the SARS-CoV-2 Spike Receptor-Binding Domain Bound to the ACE2 Receptor,” Nature, 1-9 (2020). These 6 residues were mutated individually to an amber stop codon TAG to generate soluble ACE2 expression plasmids. To test whether these positions were permissive, we co-transfected the resultant pcDNA-ACE2-D30TAG, pcDNA-ACE2-H34TAG, pcDNA-ACE2-E37TAG, pcDNA-ACE2-D38TAG, pcDNA-ACE2-Q42TAG, and pcDNA-Y83TAG plasmids respectively with pMP-FSYRS-3xC25 (which expresses the orthogonal tRNA^{Pyl}_{CUA}/FSYRS) into HEK293T cells.

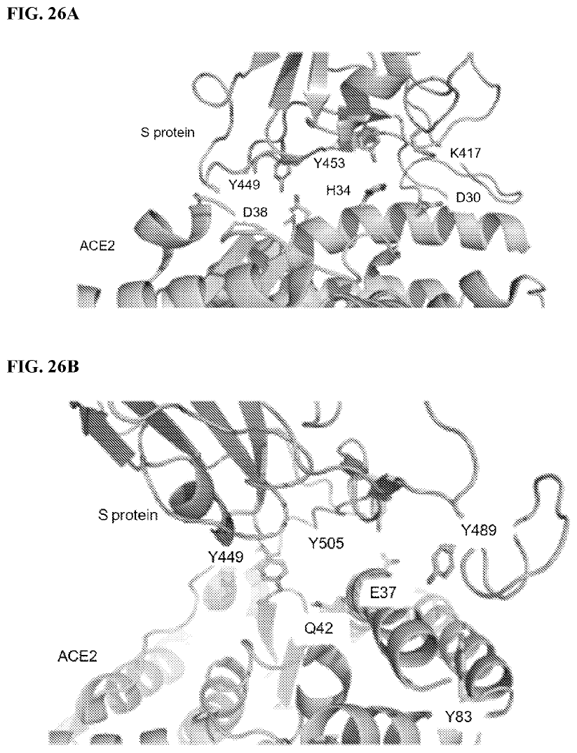

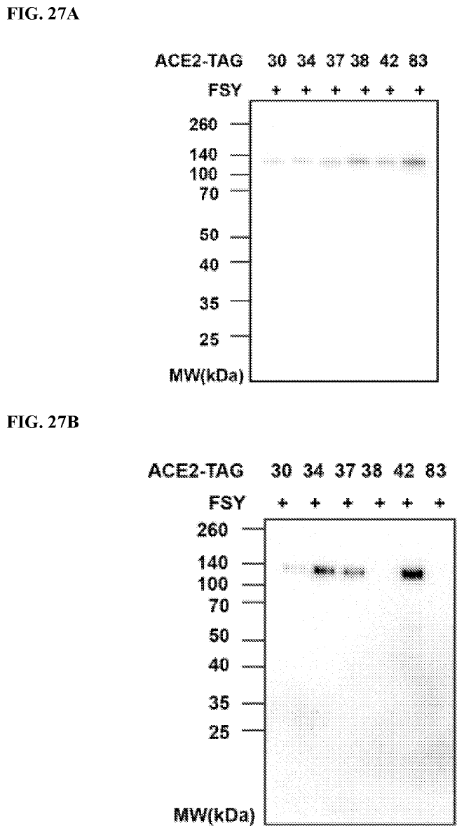

As shown in FIG. 27A, FSY was successfully incorporated into ACE2 proteins in HEK293T cells, as full-length of soluble ACE2 protein was detected in western blot. However, the protein yield was relatively low. To increase the protein yield, Expi293F cells were transiently transfected with the plasmids mentioned above and purified ACE2-FSY mutant proteins via affinity chromatography. As shown in FIG. 27B, the expression levels of ACE2-30FSY, ACE2-34FSY, ACE2-37FSY and ACE2-83FSY were significantly enhanced, but it did not work well for ACE2-38FSY and ACE2-83FSY.

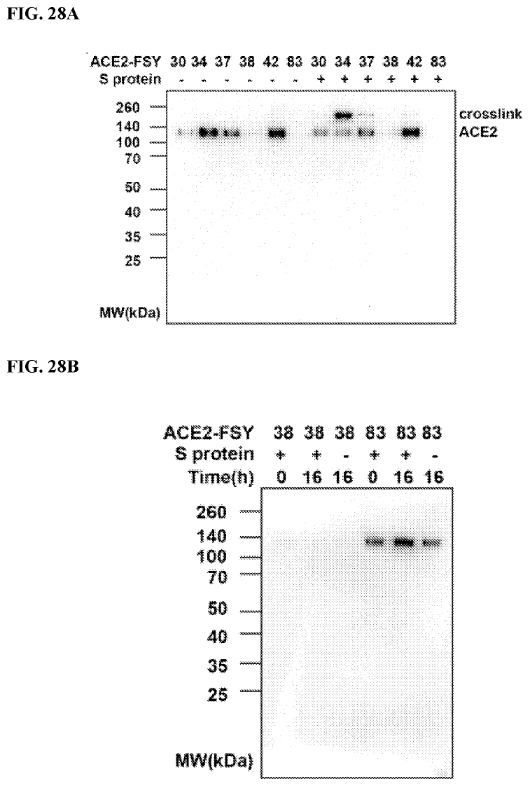

Covalently crosslinking of ACE2-FSY with the spike protein of SARS-CoV-2. To test whether ACE2-FSY could covalently crosslink with SARS-CoV-2, ACE2-FSY mutant proteins were incubated with the spike protein of SARS-CoV-2 (hereinafter referred to as “S protein”) at 37° C. for 16 hours and the crosslinking bands were detected using western blot. As shown in FIG. 28A, ACE2-34FSY, ACE2-37FSY and ACE2-42FSY formed stable crosslinking adducts with S protein with crosslinking efficiency of 89%, 22%, and 0.5%, respectively. In the negative control samples, ACE2-FSY mutants did not show any crosslinking products in the absence of the S protein, indicating that the crosslinking indeed occurred between ACE2-FSY and S protein. In a separate experiment, the transfection procedures were optimized and improved the expression of ACE2-38FSY and ACE2-83FSY (FIG. 28B). But no crosslinking products were detected for these two mutants.

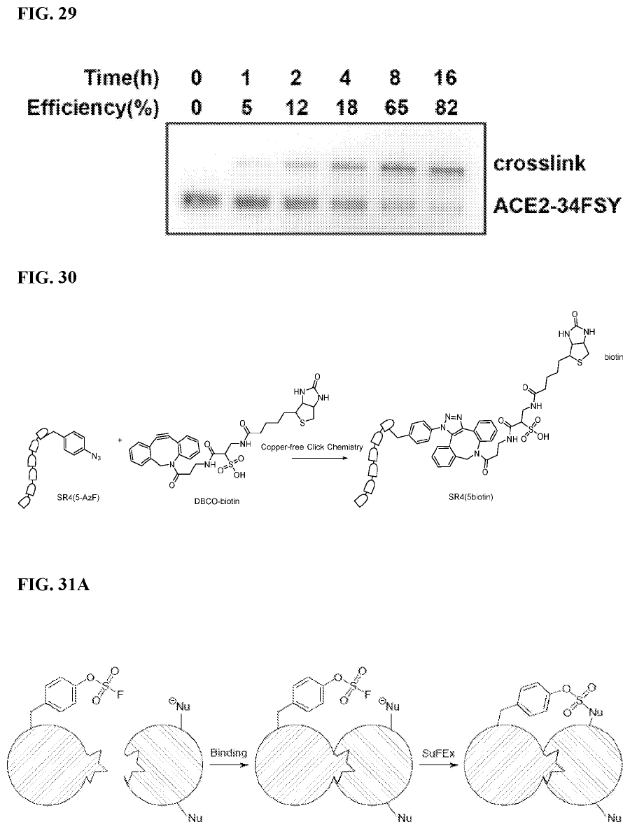

Kinetics for covalent crosslinking. To measure the covalent binding kinetics, the inventors focused on ACE2-34FSY as it presented the highest crosslinking efficiency. ACE2-34FSY was incubated with the S protein at 37° C. for 0, 1, 2, 4, 8, and 16 hours, and then the crosslinking efficiency was analyzed using Western blot. As shown in FIG. 29, the crosslinking efficiency increased along with the extending incubation time. The apparent second-order rate constant of covalent crosslinking was measured to be 7359±513 M⁻¹min⁻¹ (mean±s.d.).

### DISCUSSION

The inventors selected six amino acid residue sites of ACE2 (D30, H34, E37, D38, Q42, and Y83) for FSY incorporation, and found three sites (34, 37, and 42) could enable covalent crosslinking of ACE2-FSY with the S protein of SARS-CoV-2, among which ACE2-34FSY afforded the highest crosslinking efficiency (89%) in vitro. The ability to generate multiple covalently functional ACE2-FSY mutants will be able to prevent viral escape through mutation. Certain target residues of the S protein are essential for ACE2 binding, mutation of which will dramatically curtail viral entry. Lan et al, “Structure of the SARS-CoV-2 Spike Receptor-Binding Domain Bound to the ACE2 Receptor,” Nature, 1-9 (2020); Wrapp et al, Science, 367(6483):1260-1263 (2020); Yan et al, Science, 367(6485):1444-1448 (2020). Alternatively, a combination of ACE2-FSY mutants can be used to shut down viral escape, as simultaneous mutation of multiple residues of the S protein would abolish ACE2 binding.

In this work, the inventors expressed and purified the soluble extracellular domain of the human ACE2 receptor containing amino acids 1 to 740. Importantly, the human ACE2 protein can be shorter than what was used here and still bind the S protein in a similar manner, for example, using amino acid residues 19 to 615. Li et al, Science, 309(5742):1864-1868) Alternatively, one can also append other proteins or domain to the soluble ACE2 receptor, such as the Fc fragment to extend the half-life in vivo. Liu et al, Kidney Int, 94(1):114-125 (2018).

Through genetically incorporating a latent bioreactive Uaa into the soluble ACE2, the inventors readily converted a conventional noncovalent protein binder into a covalent protein binder, which irreversibly and covalently bind with the S protein of SARS-CoV-2 via PERx mechanism, which effectively inhibits viral infection without inducing resistance. This covalent protein drug has the following advantages: high specificity toward its target (in contrast to off-target concern for small molecule covalent drugs); direct use in vivo obviating further modification to extend its serum half-life; and dramatically enhanced therapeutic efficacy, reaching a level that is unattainable with the conventional noncovalent protein drugs.

The ACE2-FSY covalent protein drug described herein will quickly afford a medication for COVID-19 patients to prevent significant morbidities and death, and provide a prophylactic to give passive immunity to clinical providers at the front line. In addition, the PERx-capable ACE2 drugs can serve as a therapeutic stockpile for future outbreaks of SARS-CoV, SARS-CoV-2, and any new coronavirus that use the ACE2 receptor for entry.

### Methods

### Construction of ACE2 Expression Vectors

The 1 to 740 aa of human ACE2 gene with His-6 tag appended at the C terminus was amplified by PCR using primers SEQ ID NO:6 and SEQ ID NO:7. Subsequently, the PCR products were purified and ligated into HindIII/BamHI sites of pcDNA 3.1 vector to generate pcDNA-ACE2-His by homologous recombination with Vazyme ClonExpress II One Step Cloning kit following the manufacturer's instructions.

For FSY incorporation, a single amber TAG mutation was placed into pcDNA-ACE2-His plasmid to generate pcDNA-ACE2-D30TAG, pcDNA-ACE2-H34TAG, pcDNA-ACE2-E37TAG, pcDNA-ACE2-D38TAG, pcDNA-ACE2-Q42TAG, and pcDNA-Y83TAG plasmids by site directed mutagenesis with the primers of SEQ ID NOS:8-19.

Incorporation of FSY into ACE2

One day before transfection, 4.5×10⁴ HEK293T cells were seeded in a Greiner bio-one 24 well-cell culture dish containing 500 μL of DMEM media with 10% FBS, and incubated at 37° C. in a CO₂ incubator. 500 ng of pMP-FSYRS-3xC25 and 500 ng of pcDNA-ACE2-D30TAG, pcDNA-ACE2-H34TAG, pcDNA-ACE2-E37TAG, pcDNA-ACE2-D38TAG, pcDNA-ACE2-Q42TAG, or pcDNA-Y83TAG plasmids were cotransfected into target cells using 3 μL PEI transfection reagent (40 kDa, 1 mg/mL) following a standard protocol. 48 h post-transfection, the supernatant was collected for western-blot analysis.

### Purification of ACE2-FSY Proteins

One day before transfection, Expi293F cells (ThermoFisher) were seeded at a final density of 1.5-2×10⁶ viable cells/mL in 10 mL Freestyle™ 293 Medium (ThermoFisher) and then grown overnight. 10 μg of pMP-FSYRS-3xC25 and 10 μg of pcDNA-ACE2-D30TAG plasmids were diluted in 250 μL Opti-MEM media (Thermo Fisher) to make Opti-MEM-DNA solution. 60 μL of PEI transfection reagent (40 kDa, 1 mg/mL) was diluted in 250 μL Opti-MEM media to make Opti-MEM-PEI solution. Then Opti-MEM-DNA and Opti-MEM-PEI solutions were mixed by vigorous vortex and incubated at ambient temperature for 30 min. The transfection complex was then added to Expi293F cells. FSY solution was supplemented at a final concentration of 1 mM to the cell culture 1 h post-transfection. 48 h post-transfection, the supernatant which contained ACE2-30FSY protein was collected by centrifugation at 1000 g×5 min for twice to remove as many cells as possible. Then the supernatant was diluted with 10 mL of Tris washing buffer (50 mM Tris-HCl pH 8.0, 500 mM NaCl, 20 mM Imidazole), and then incubated with 300 μL of pre-equilibrated Protino Ni-NTA Agarose (MACHEREY-NAGEL) at 4° C. for 1h. Then Ni-NTA Agarose was washed with 500 μL of Tris washing buffer for 3 times, and eluted with 150 μL of Tris elution buffer (50 mM Tris-HCl pH 8.0, 500 mM NaCl, 250 mM Imidazole) for 3 times. The elution was combined and buffer exchanged to 1×PBS, pH=7.4 with an 30K Amicon® Ultra Centrifugal Filters (Millipore Sigma). ACE2-34FSY, ACE2-37FSY, ACE2-38FSY, ACE2-42FSY, and ACE2-83FSY proteins were purified following the same procedures.

### Crosslinking Between ACE2-FSY and Spike RBD of SARS-CoV-2

SARS-CoV-2 (2019-nCoV) Spike RBD-mFc Recombinant Protein (hereinafter referred to as “S protein”) was purchased from Sino Biological. 8 μL of ACE2-FSY protein (0.01-0.03 μg/uL in PBS, pH=7.4) was mixed with 0.5 μL of S protein (0.5 μg/pL in PBS, pH=7.4) and incubated at 37° C. for 16 h. As a negative control, 8 μL of ACE2-FSY protein (0.01-0.03 μg/μL in PBS, pH=7.4) was mixed with 0.5 μL of PBS, pH=7.4 and incubated at 37° C. for 16h. For the kinetic study, 8 μL of ACE2-FSY protein (0.01-0.03 μg/μL in PBS, pH=7.4) was mixed with 0.5 μL of S protein (0.5 μg/μL in PBS, pH=7.4) and incubated at 37° C. for 0, 1, 2, 4, 8, and 16 h.

### Example 7

The inventors previously demonstrated that bioreactive unnatural amino acids (Uaas) bearing electrophilic moieties are able to selectively react with nucleophilic amino acid upon binding in a proximity-enabled manner, providing the specificity to capture protein-protein interactions. (Ref 1). Herein, we genetically encoded the latent bioreactive Uaa, fluorosulfate-L-tyrosine (FSY), into different nanobodies that specifically bind to the receptor binding domain (RBD) of the SARS-CoV-2 viral spike protein, converting the nanobodies into covalent binders for the spike protein in order to inhibit SARS-CoV-2 infection of human cells (FIG. 24). (Ref. 2)

### Results

Genetically Encode FSY into Nanobody to Generate Covalent Nanobody Targeting the Spike RBD.

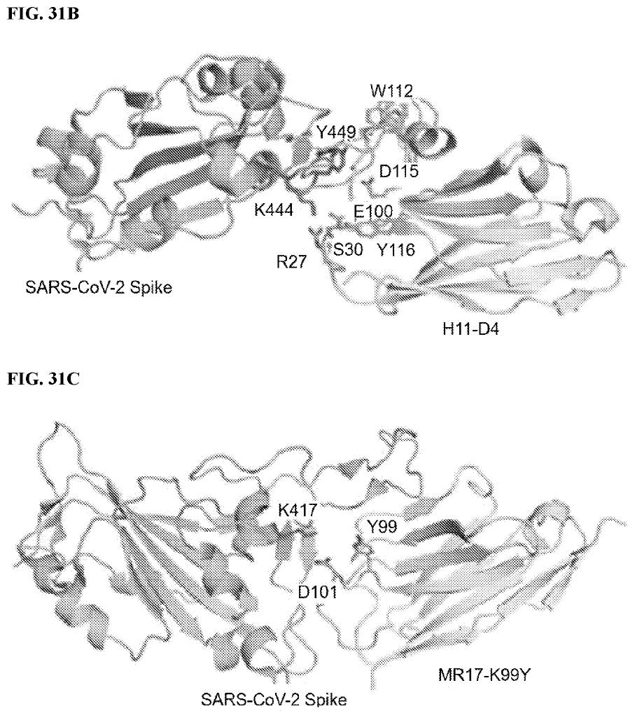

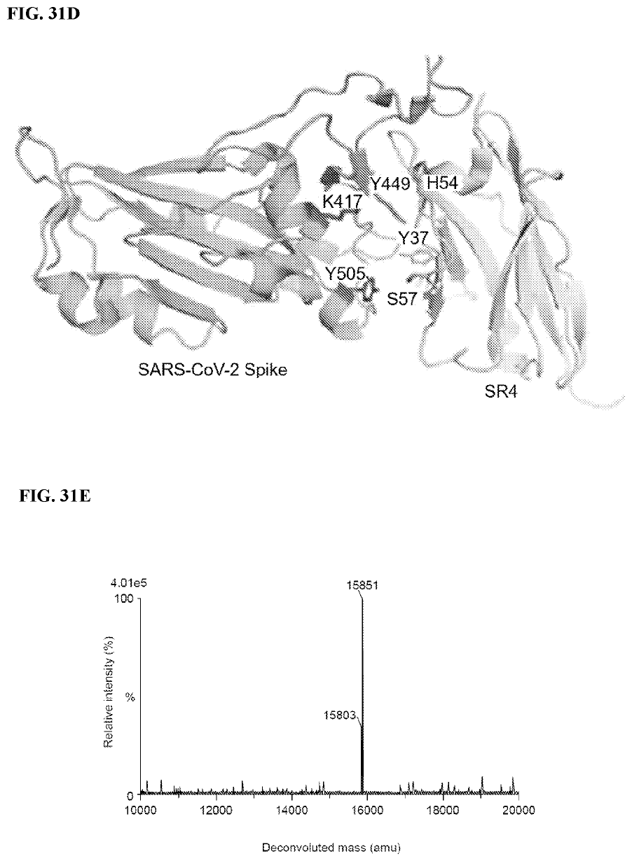

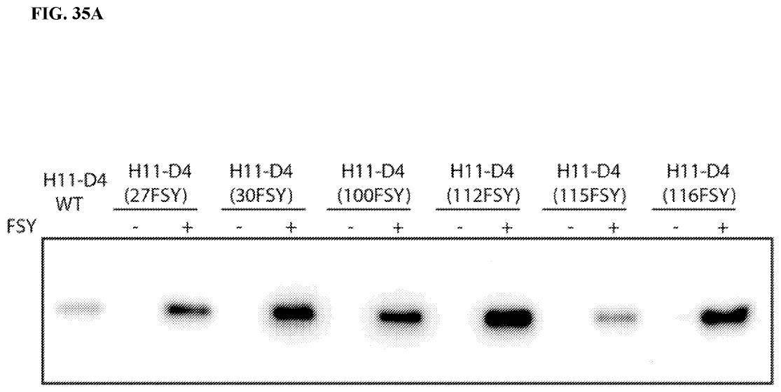

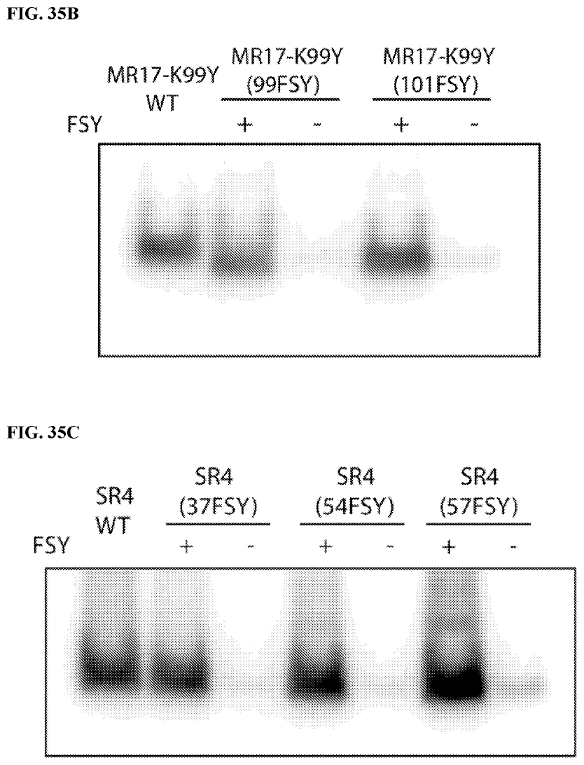

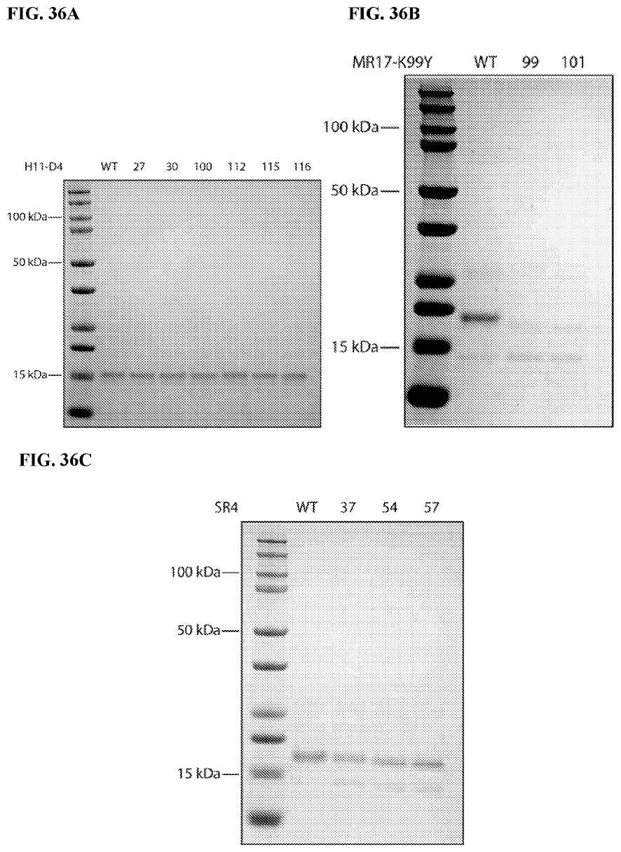

The incorporation of FSY into nanobodies will convert the nanobodies into covalent protein drugs to block the endogenous interaction between SARS-CoV-2 (2019-nCoV) spike RBD and human ACE2 receptor. On the basis of the crystal structure of human SARS-CoV-2 spike RBD/H11-D4 complex, human SARS-CoV-2 spike RBD/MR17-K99Y complex and human SARS-CoV-2 spike RBD/SR4 complex, we incorporated FSY at site R²⁷, S30, E100, W112, D115, or Y116 of nanobody H11-D4 (FIG. 31B), site Y99 or D101 of nanobody MR17-K99Y (FIG. 31C), site Y37, H54 or S57 of nanobody SR4 (FIG. 31D), respectively. (Refs: 3,4). Western blot analysis of the cell lysates of cells expressing these mutant nanobodies genes with or without FSY in the culture confirmed that FSY was successfully incorporated into the nanobodies in the presence of FSY (FIGS. 35A-35C). WT and FSY-incorporated nanobodies were purified with Ni²⁺ or Co²⁺ affinity beads with good purity (FIGS. 36A-36C). Mass spectrometric analysis of the intact protein confirmed that FSY was incorporated into nanobody SR4 at site 57 in high fidelity (FIG. 31E).

### FSY-Incorporated Nanobodies Selectively Bind to SARS-CoV-2 Spike RBD in Covalent Mode In Vitro

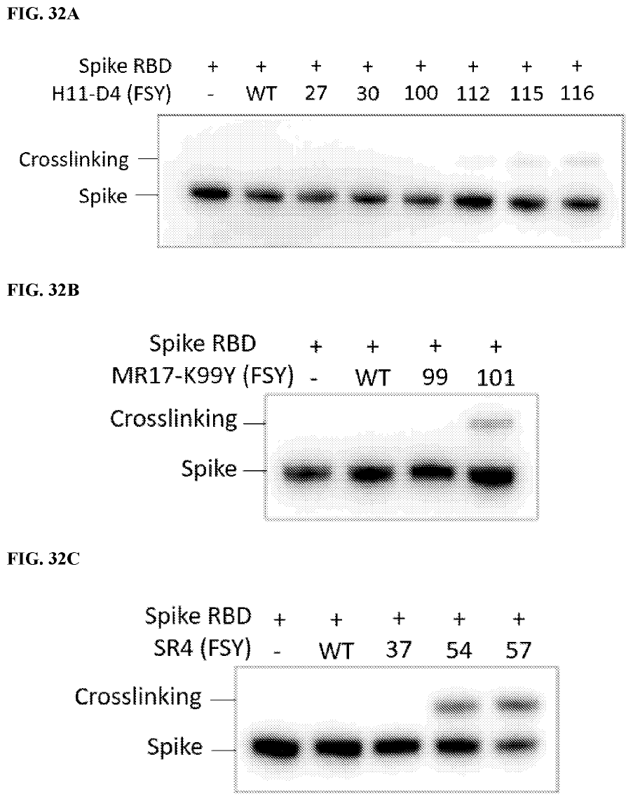

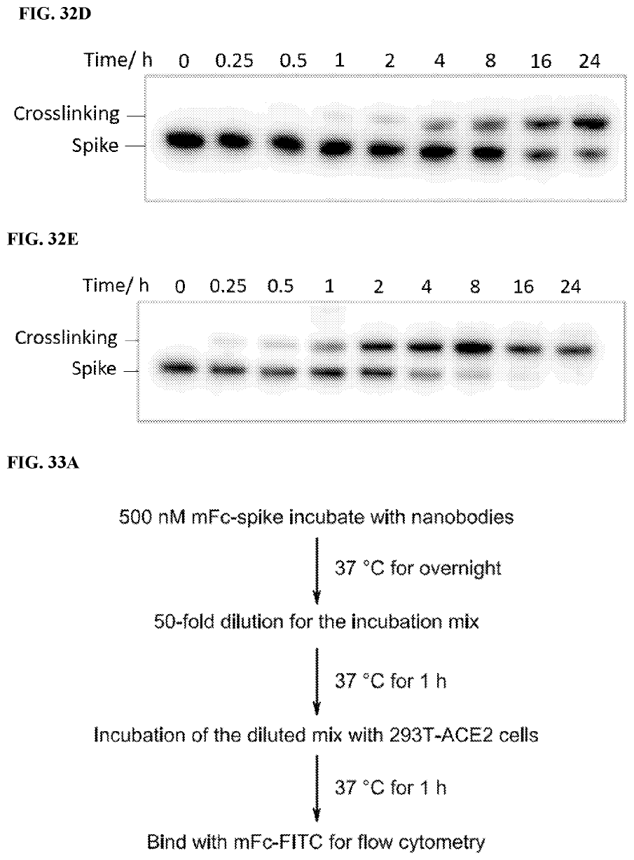

To test if FSY-incorporated nanobody mutants could bind to spike RBD covalently in vitro, we incubated these WT nanobodies and their FSY mutant proteins with the spike RBD at 1:5 molar ratio at 37° C. for overnight, followed by Western blot analysis against mouse Fc (mFc), which is appended at the C-terminus of the Spike RBD (FIGS. 32A-32C). H11-D4 (WT), H11-D4 (27FSY), H1T-D4 (30FSY), HT1-D4 (100FSY), and H11-D4 (112FSY) did not form a covalent complex with spike RBD, whereas a stable covalent complex was detected for H11-D4 (115FSY) and H11-D4 (116FSY) with low crosslinking efficiency (FIG. 32A). MR17-K99Y (WT) and MR17-K99Y (99FSY) did not form a covalent complex with spike RBD, whereas a stable covalent complex was detected for MR17-K99Y (101FSY) with 10.5% crosslinking efficiency (FIG. 32B). SR4 (WT) and SR4 (37FSY) did not form a covalent complex with spike RBD, whereas a stable covalent complex was detected for SR4 (54FSY) and SR4 (57FSY), with SR4 (54FSY) showing 28.3% crosslinking efficiency, and SR4 (57FSY) showing 41.3% crosslinking efficiency (FIG. 32C). According to above results, we studied the crosslinking dynamics of SR4 (54FSY) and SR4 (57FSY) nanobodies which had higher crosslinking efficiencies. 5 μM nanobodies were incubated with 0.5 μM spike RBD at 37° C. for different time length, the reaction was terminated and subjected for Western blot analysis against mFc (FIGS. 32D-32E). Based on the faster reaction kinetics of SR4 (57FSY), we used SR4 (57FSY) for subsequent experiments.

Nanobody(FSY) Inhibits RBD Binding to Cell Surface ACE2 Receptor More Effectively than WT Nanobody

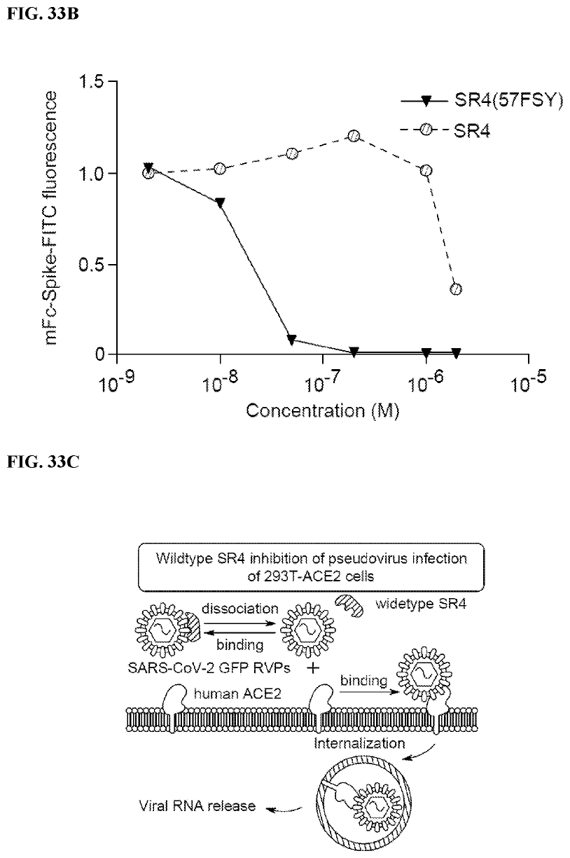

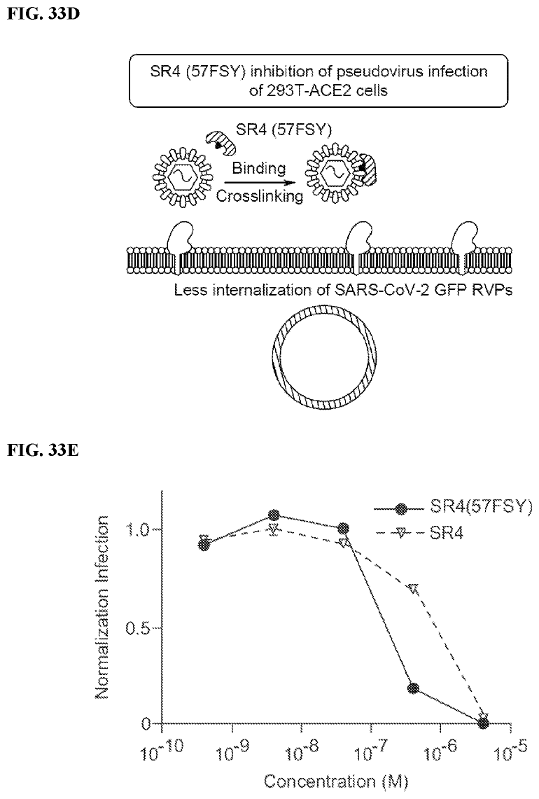

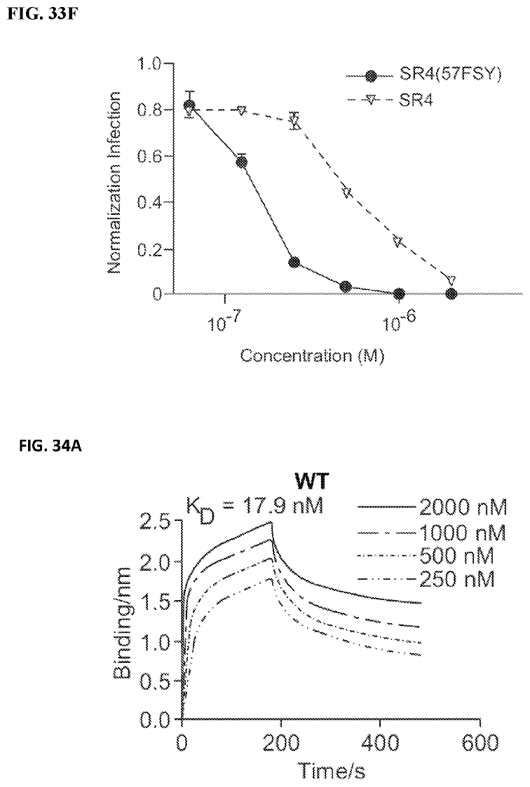

We next tested the efficacy of our nanobodies to inhibit the binding of mFc-spike RBD to 293T-ACE2 cells, a HEK293T cell line stably expressing human hsACE2 protein on cell surface. Different concentrations of SR4 or SR4 (57FSY) were individually incubated with mFc-spike RBD at 37° C. overnight to allow cross-linking, followed by the incubation with 293T-ACE2 cells. After incubation, cells were stained with anti-mFc-FITC and analyzed with flow cytometry (FIG. 33A). SR4 could not crosslink with the spike RBD, so the spike RBD could still bind to ACE2 on cell surface, resulting in high mean fluorescence intensity (MFI). In contrast, SR4 (57FSY) showed highly efficient blocking of the spike RBD binding to 293T-ACE2 cells (FIG. 33B). The IC₅₀ of SR4 (57FSY) was about 100 fold lower than that of SR4.

Nanobody(FSY) Neutralizes Pseudovirus Infection of 293T-ACE2 Cells More Effectively than WT Nanobody

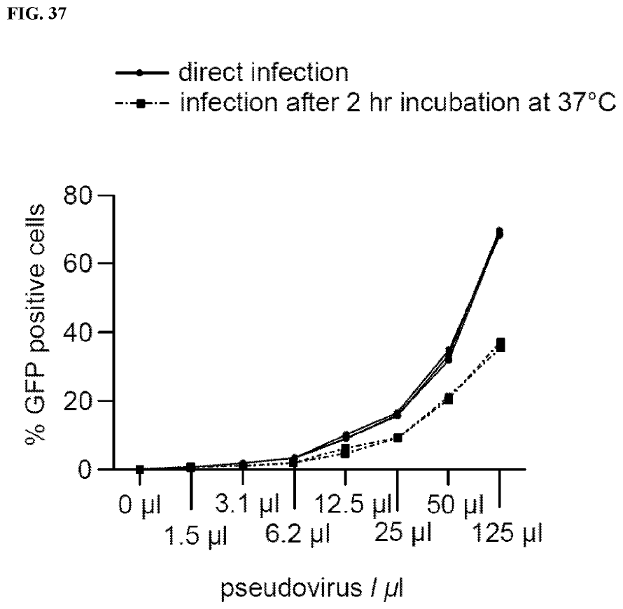

We next tested the neutralization activity of SR4 or SR4 (57FSY) nanobodies against SARS-CoV-2 pseudotyped lentivirus (FIGS. 33C-33D). SARS-CoV-2 reporter virus particles (RVPs) display antigenically correct spike protein on a heterologous virus core and carry a modified genome that expresses a convenient GFP reporter gene, which is integrated and expressed upon successful viral entry into cells harboring the ACE2 receptor. We first tested the infectivity of pseudovirus. Different volumes of pseudovirus were used to infect 293T-ACE2 cells after directly thawing at 37° C. for 3 min or after 2 h incubation at 37° C. The cell infection percentage increased from 0 to 70% as pseudovirus amount increased, and the pseudovirus infectivity decreased around 50% after 2 h incubation at 37° C. (FIG. 37). Initially, various concentrations of SR4 or SR4-57FSY were incubated with 40 μL pseudovirus for 2 h, followed by ten times dilution at 37° C. for 1 h and incubation with 293T-ACE2 cells. Cells were harvested for fluorescence-activated cell sorting (FACS) analysis to determine the percentage of cell infection via GFP positive signal. The results showed that 4 μM SR4 or SR4(57FSY)/pseudovirus mix almost completely inhibited pseudovirus infection of 293T-ACE2 cells with no difference (FIG. 33E, left). 0.4 μM SR4(57FSY) had 20% cell infection compared with 80% infection with 0.4 μM WT SR4 (FIG. 33E). Lower concentrations such as 0.04 μM and 0.004 μM SR4 and SR4-57FSY didn't show inhibition effects. We then performed the inhibition experiments with nanobody concentrations in the range of 62.5 nM to 2 μM. (FIG. 33F). The IC₅₀ was measured 0.55 μM for SR4 and 0.16 μM for SR4(57FSY), demonstrating that the covalent SR4(57FSY) was more effective in inhibiting pseudovirus infection of ACE2-expressing human cells than SR4.

### SR4 (57FSY) Binds to SARS-CoV-2 Mutant Spike RBD in Covalent Mode In Vitro

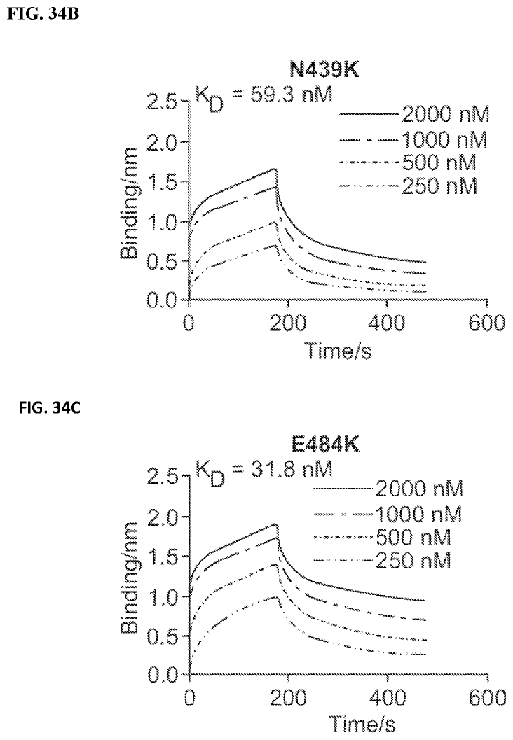

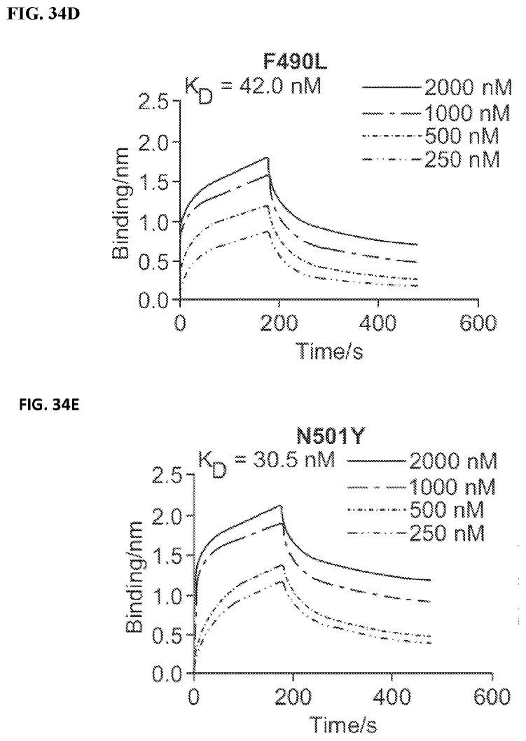

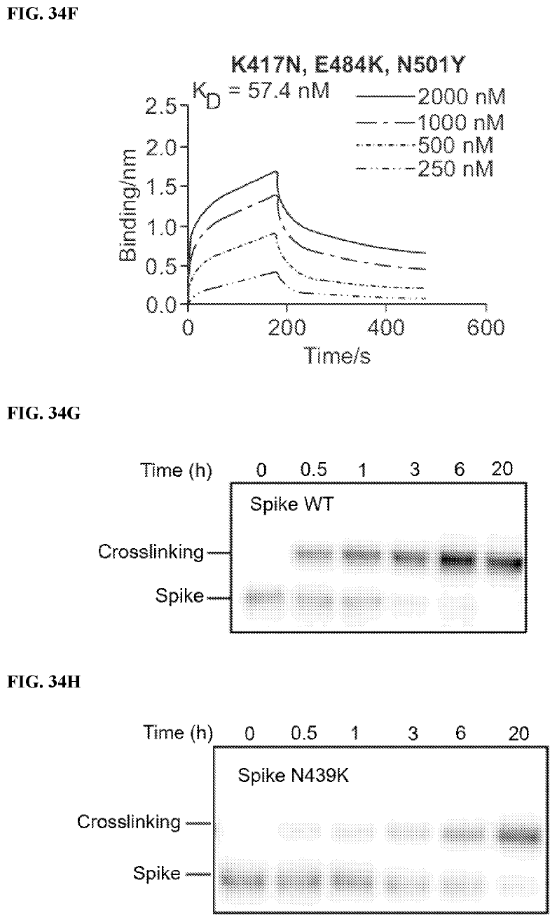

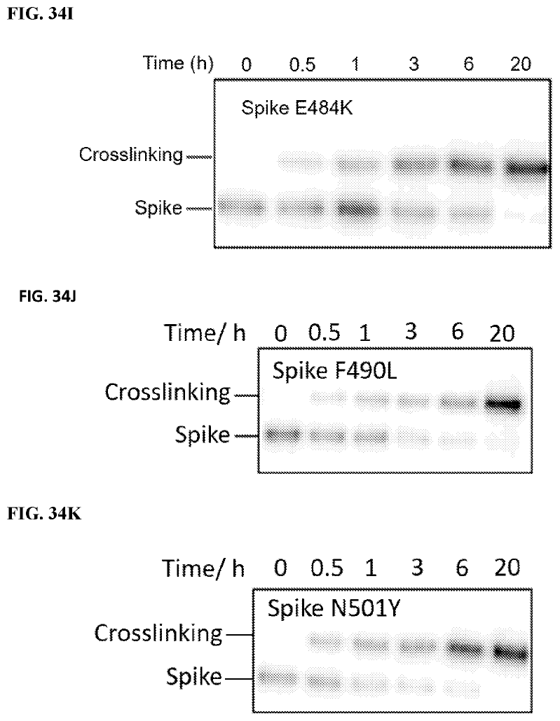

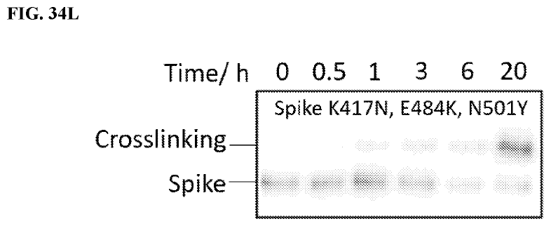

Various mutated SARS-CoV-2 strains have emerged in the pandemic. Mutations on the spike RBD such as E484K, F490L and N439K have been shown to have decreased affinity towards neutralization antibodies. (Refs. 6-8). The B.1.1.7 lineage, which mainly possess N501Y mutation, shows a stronger interaction with ACE2 and a faster spreading rate. (Refs. 8-9). The B.1.351 lineage, which have K417N, E484K, and N501Y mutations on the spike RBD, has decreased affinity towards neutralization antibodies and can lower the effectiveness of current vaccines. (Refs: 10-13). We first determined the binding constant (K_{D}) between mutated spike proteins and the SR4 nanobody using biolayer interferometry (BLI). As shown in FIGS. 34A-34F and Table 4, all mutated spike proteins had decreased affinity towards the SR4 nanobody.

TABLE 4

| Spike RBD | K_{D} (nM) | Kon (1/Ms) | Koff (1/s) |
|---|---|---|---|
| WT | 17.9 | 1.26 × 10⁵ | 2.26 × 10⁻³ |
| N501Y: UK strain | 30.5 | 8.86 × 10⁴ | 2.70 × 10⁻³ |
| F490L | 42.0 | 1.13 × 10⁵ | 4.76 × 10⁻³ |
| N439K | 59.3 | 1.00 × 10⁵ | 5.95 × 10⁻³ |
| E484K | 31.8 | 1.07 × 10⁵ | 3.40 × 10⁻³ |
| K417N, E484K, N501Y: | 57 | 7.08 × 10⁴ | 4.06 × 10⁻³ |
| South Africa strain | | | |

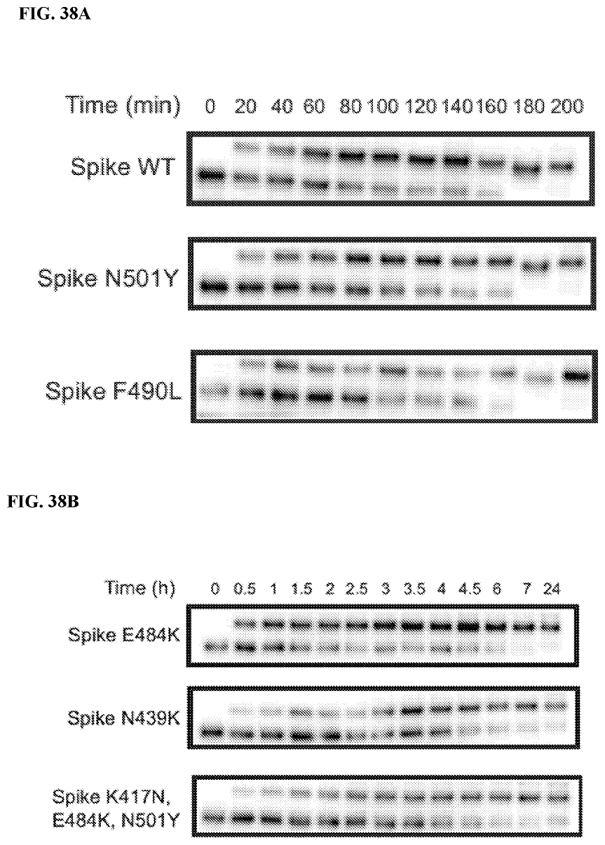

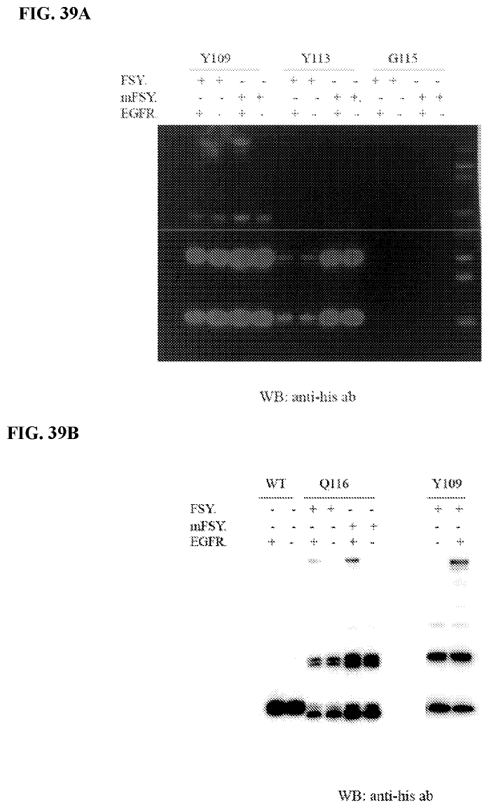

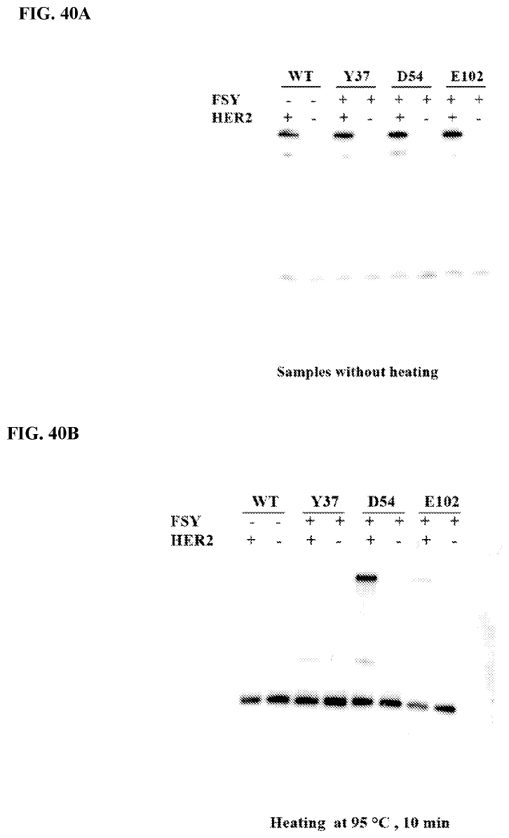

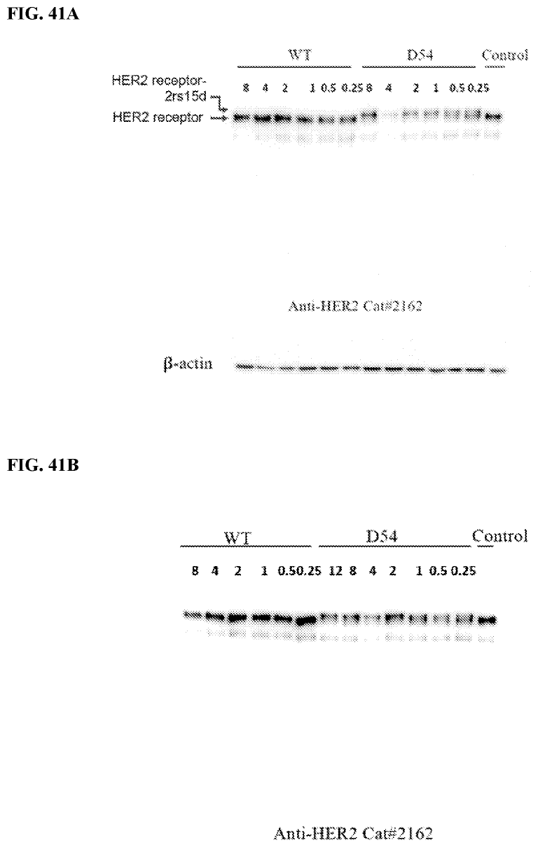

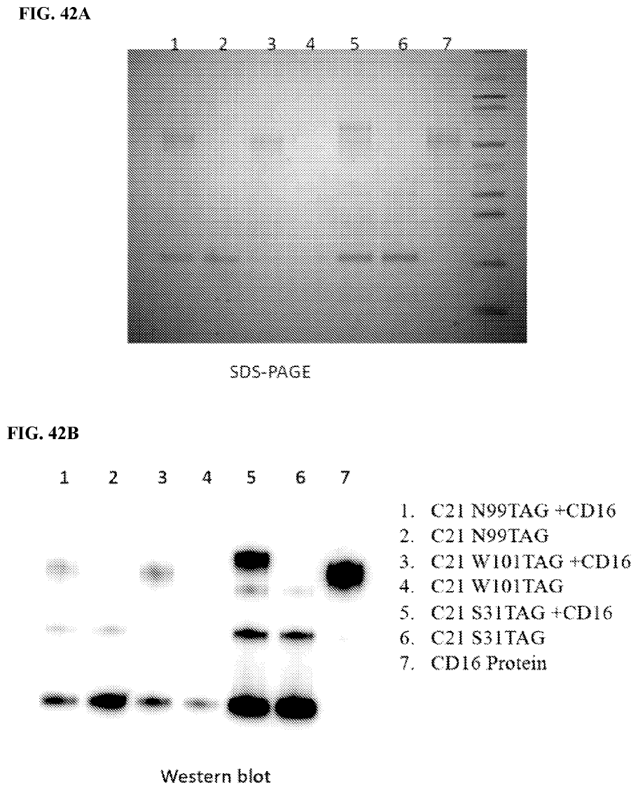

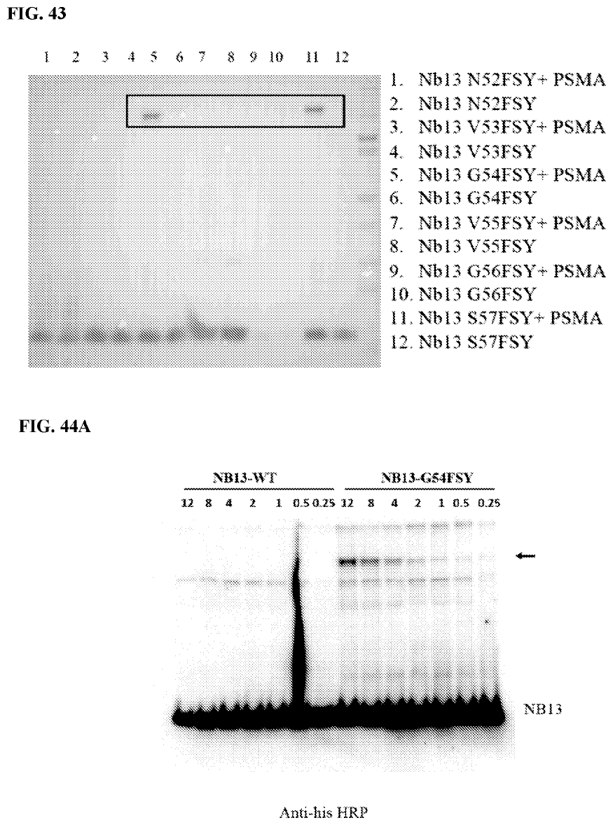

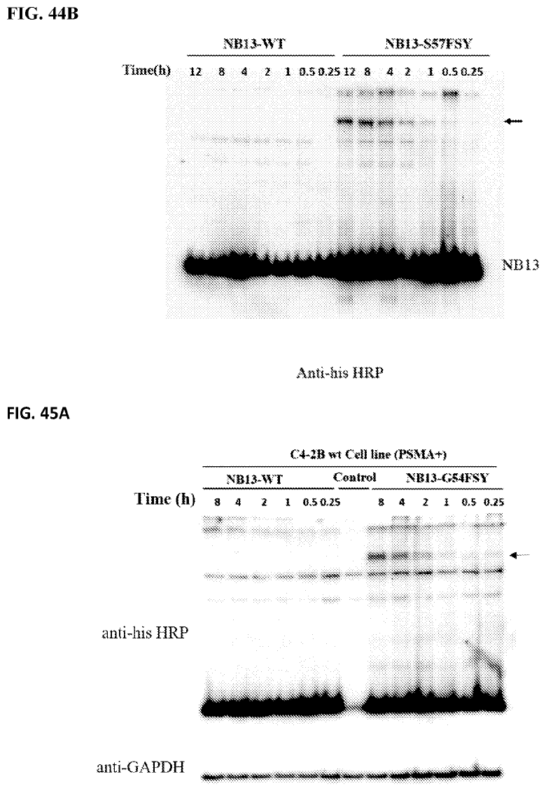

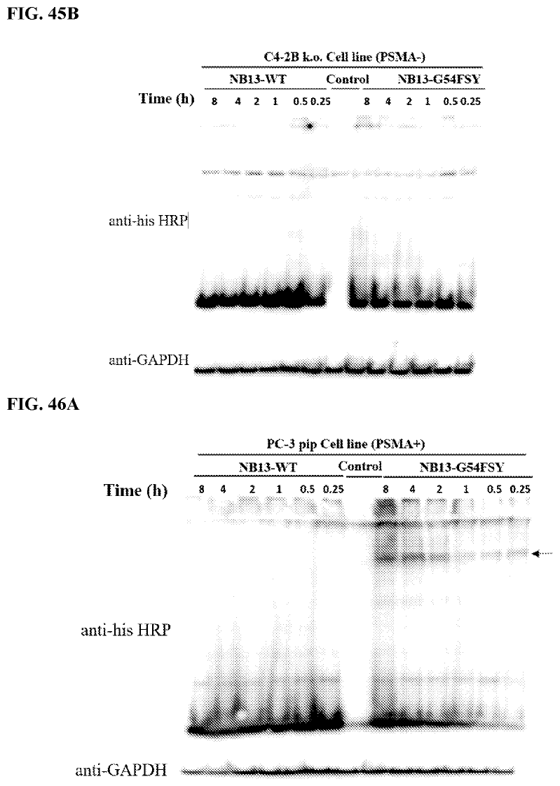

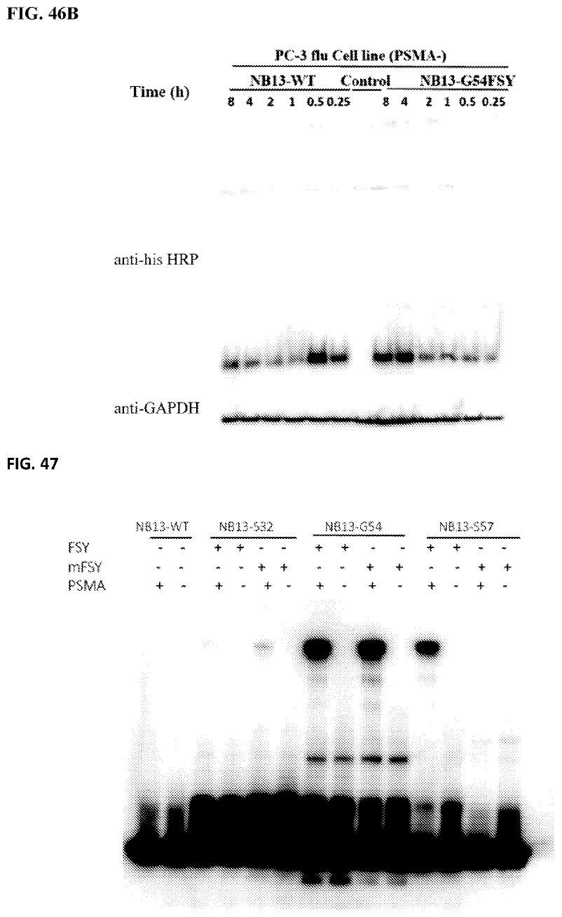

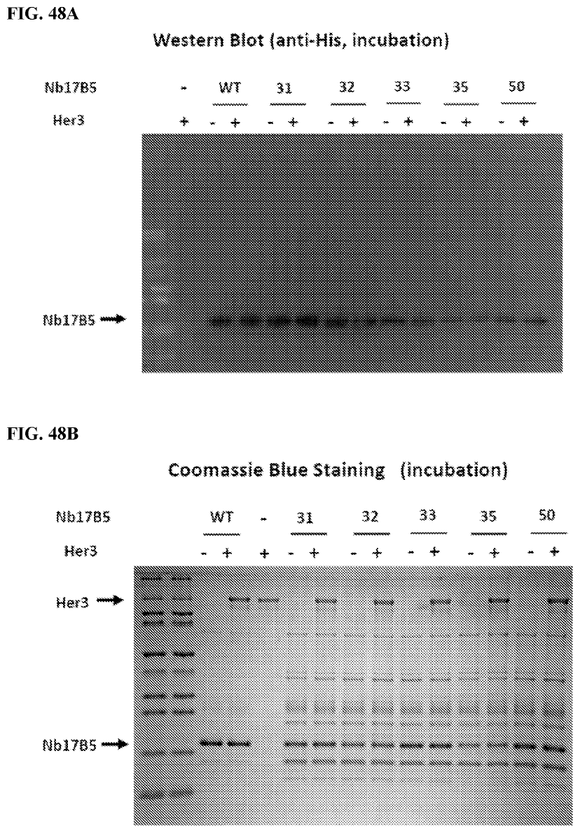

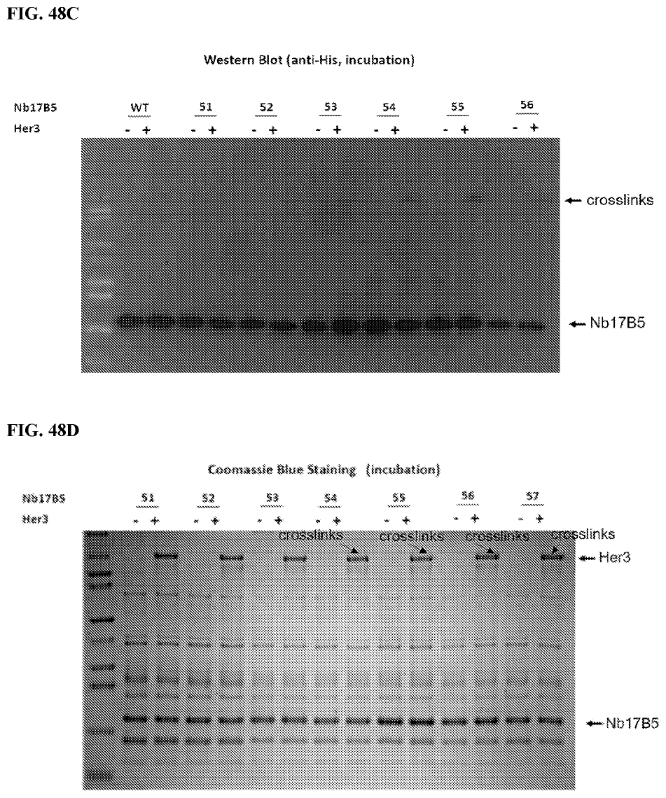

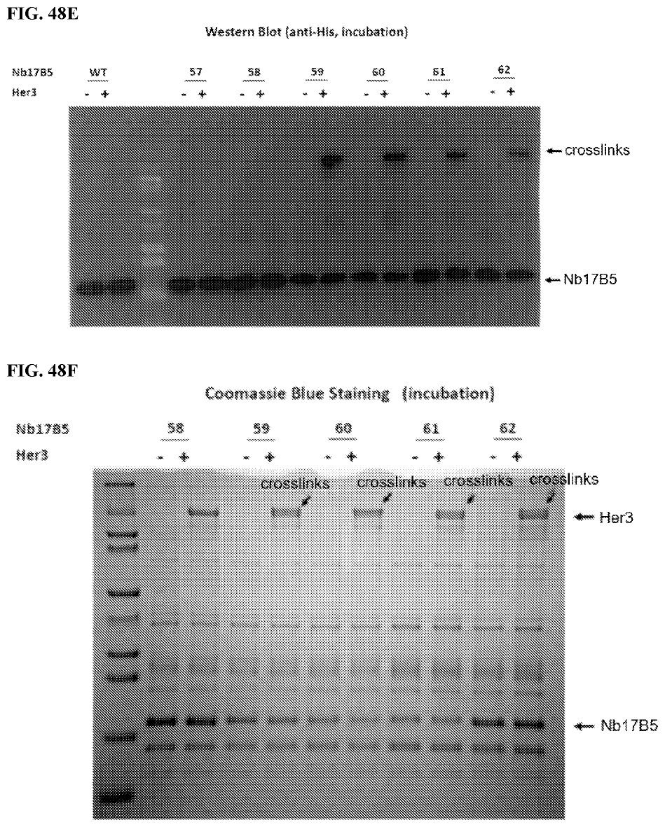

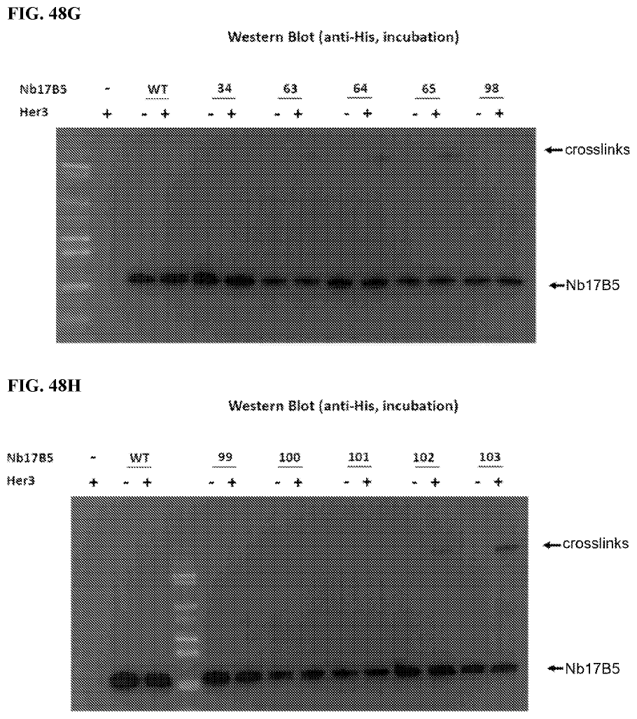

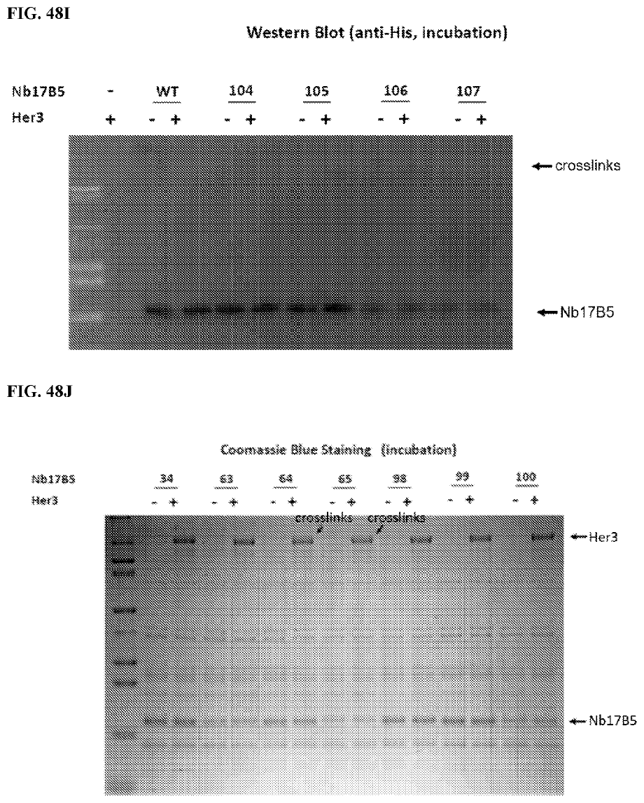

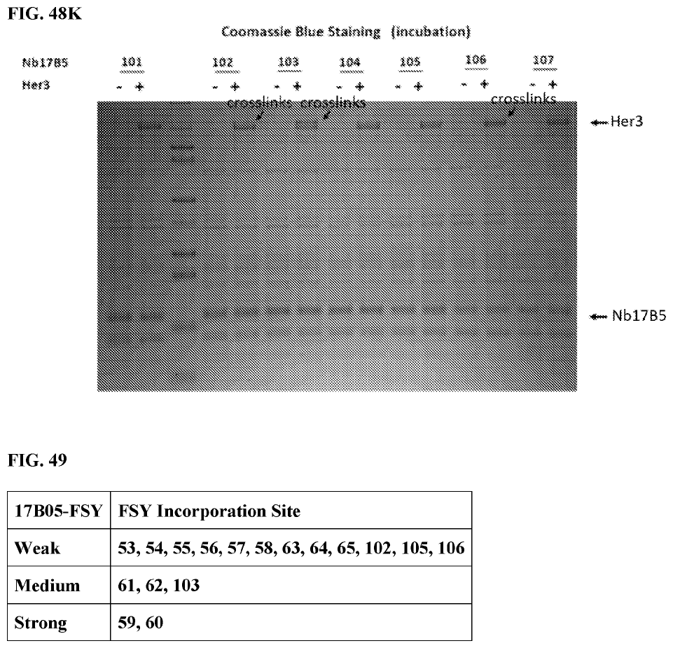

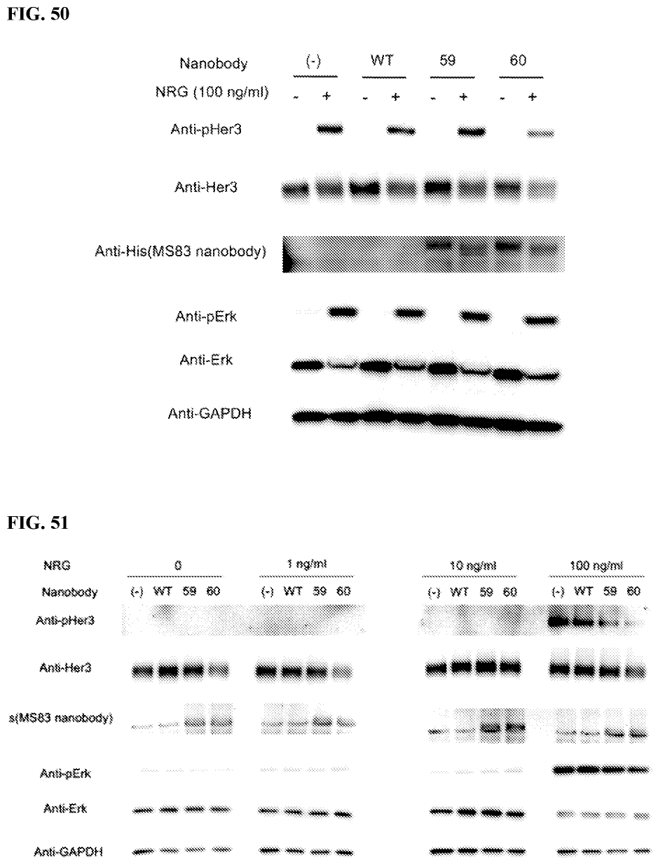

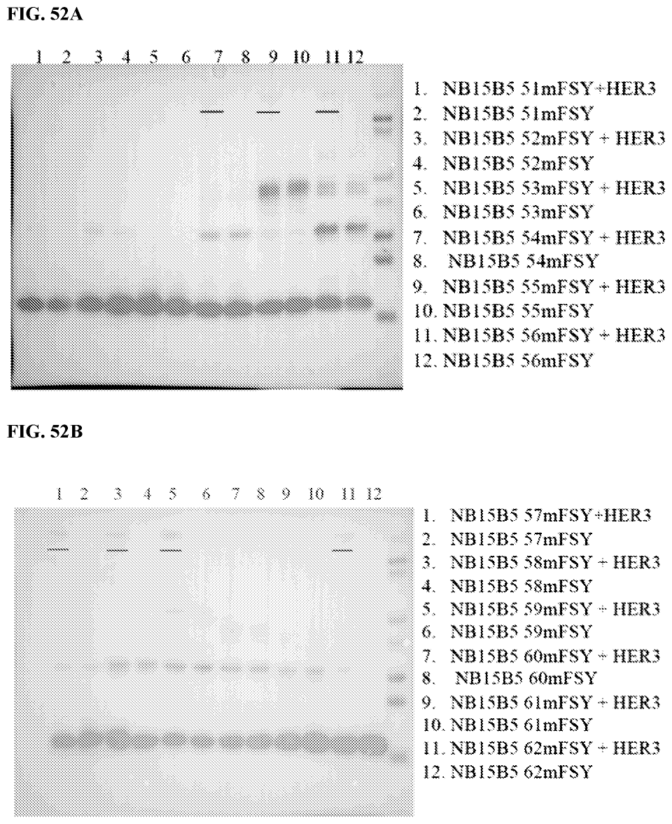

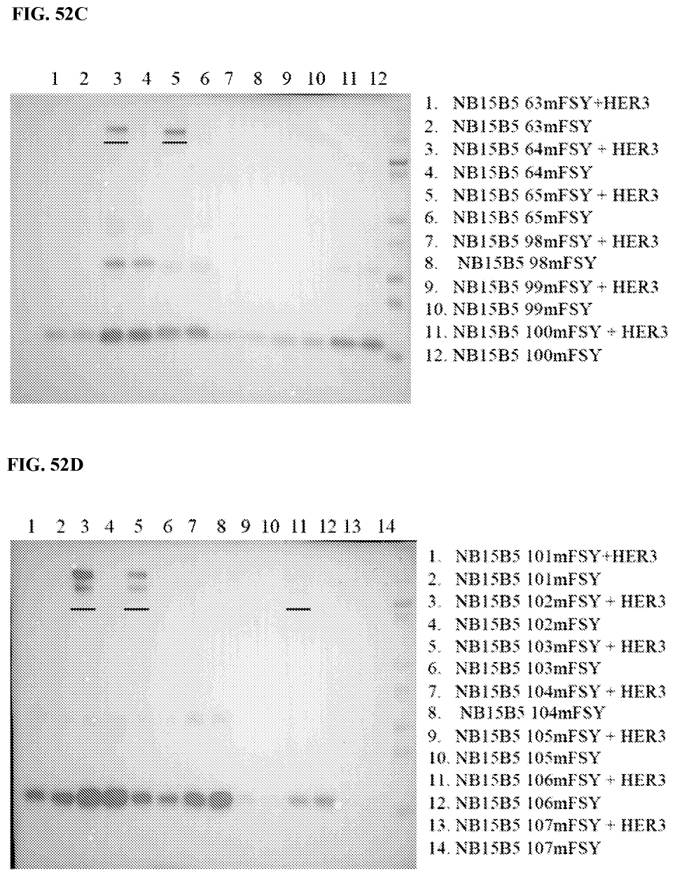

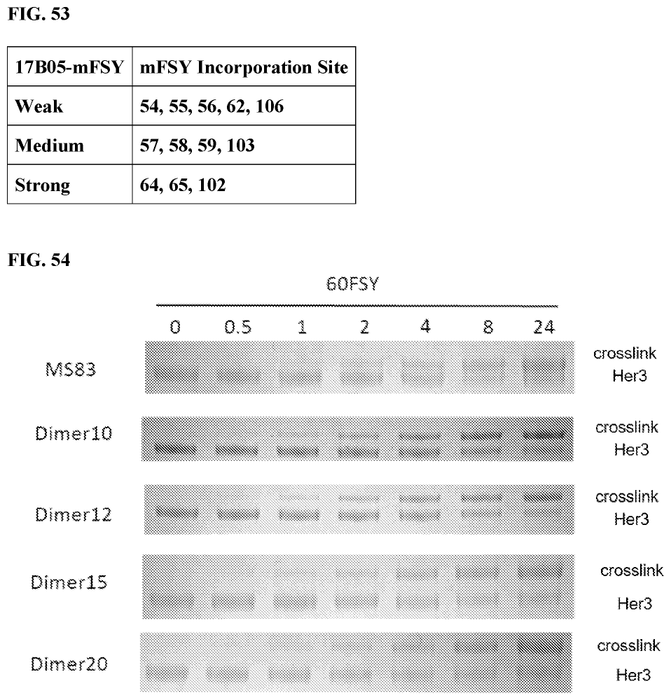

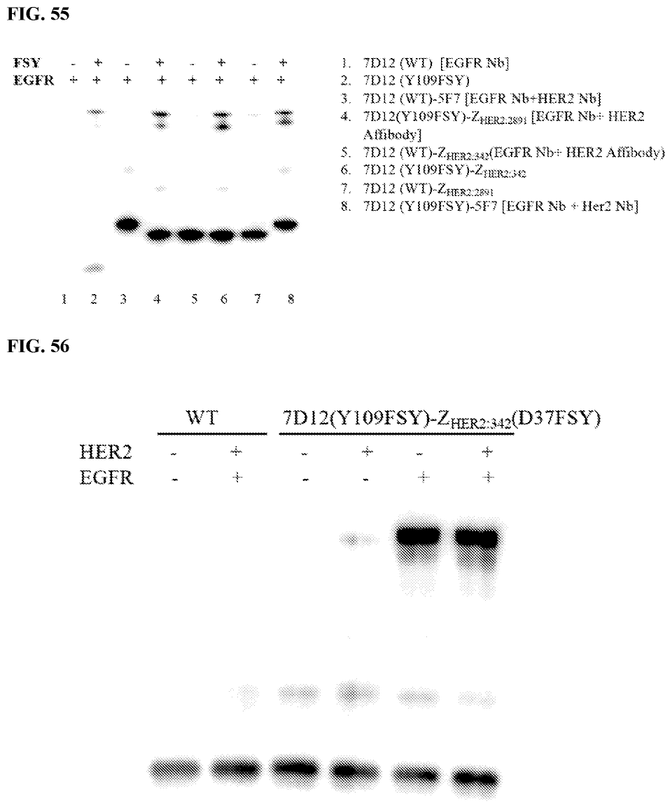

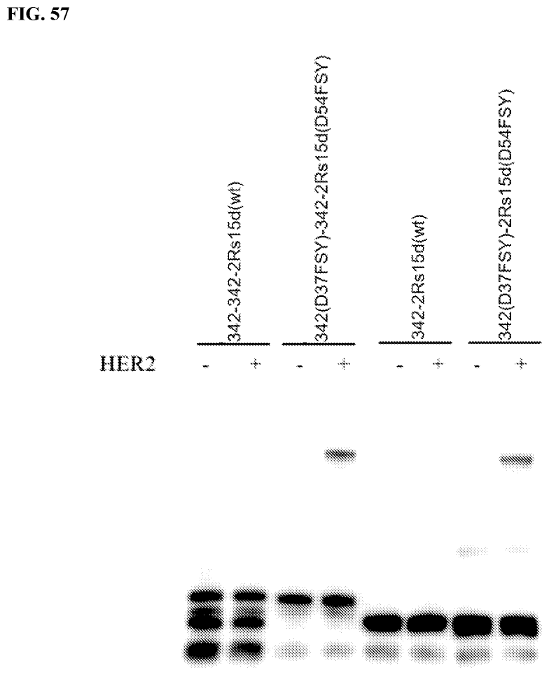

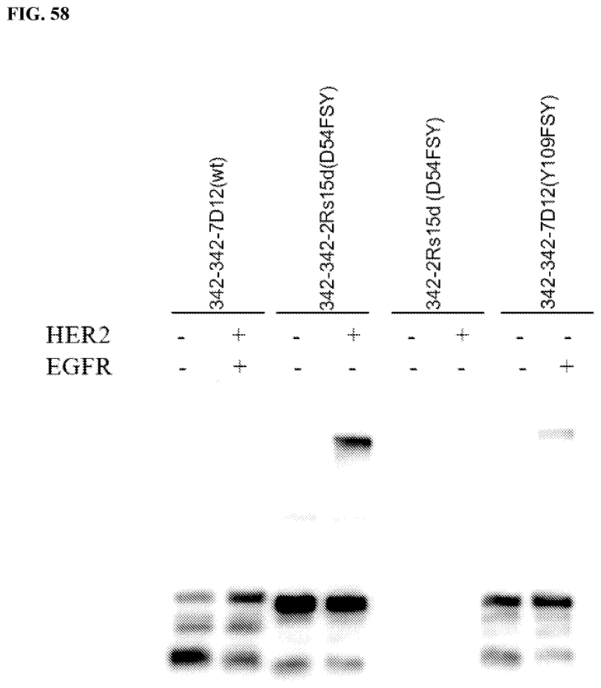

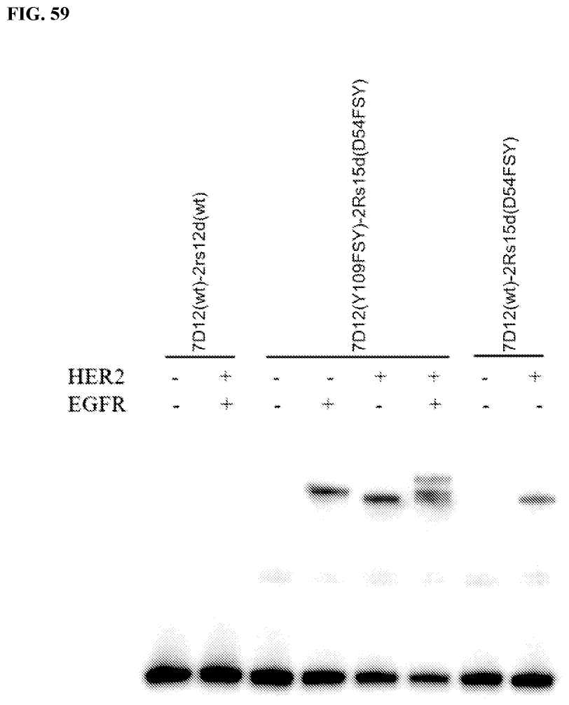

To examine if the decreased affinity could decrease cross-linking rates between the mutated spike RBD and SR4(57FSY), four spike proteins with single mutations on RBD (N501Y, F490L, N439K, and E484K) and one Spike protein with three mutations on RBD (B.1.351 lineage, K417N, E484K and N501Y) were used for the test. 0.5 μM Spike protein (wildtype or mutated) was incubated with 1.5 μM SR4 (57FSY) at 37° C. in PBS. At different time points (0 h, 0.5 h, 1 h, 3 h, 6 h and 20 h), an aliquot was extracted and the cross-linking between spike and SR4(57FSY) was examined by Western blot. All five mutated Spike proteins formed covalent adducts with SR4(57FSY) efficiently (FIGS. 34G-34L). We further characterized the cross-linking kinetics in more detail. 0.5 μM Spike protein (WT or mutants) was incubated with 5 μM SR4(57FSY) at 37° C. in PBS. At different time points, 1 μl sample was extracted, mixed with 4 μl loading buffer for western-blot analysis. The analysis results are shown in FIGS. 38A-38B. The cross-linking rate constant for each spike protein was calculated and listed in Table 2. Regarding the crosslink reaction rates between spike RBD (WT and mutated) with SR4(57FSY): N501Y has same reaction rate with WT; F490L is slightly slower than WT; other mutated spike are about 2-fold slower than the WT

TABLE 2

| Spike RBD | K_{D} (nM) | K_{obs} (M⁻¹) | K₂ (M^{-1h-1}) |
|---|---|---|---|
| WT | 17.9 | 0.595 ± 0.013 | 119000 ± 2600 |
| N501Y: UK strain | 30.5 | 0.608 ± 0.088 | 121600 ± 17600 |
| F490L | 42.0 | 0.392 ± 0.083 | 78400 ± 16600 |
| N439K | 59.3 | 0.236 ± 0.023 | 47200 ± 4600 |
| E484K | 31.8 | 0.314 ± 0.010 | 62800 ± 2000 |
| K417N, E484K, N501Y: | 57 | 0.291 ± 0.021 | 58200 ± 4200 |
| South Africa strain | | | |

### Methods

### Key Reagents

The SARS-CoV-2 GFP Reporter Virus Particles (RVPs) were purchased form Integral Molecular, Inc. (Integral Cat #RVP-701G). SARS-CoV-2 (2019-nCoV) Spike RBD-mFc Recombinant Protein was purchased from SinoBiological company (Cat #40592-V05H). Mutated SARS-CoV-2 (COVID-19) S proteins (RBD) were purchased from ACROBiosystems. Five mutated spike proteins were tested: 1. E484K (#SRD-C52H3); 2. F490L (#SRD-C52Hf); 3. N501Y (#SRD-C52Hn); 4. N439K (#SRD-C52Hg); 5. South African strain (K417N, E484K, N501Y, #SPD-C52Hp).

### Cell Culture

The 293T-hsACE2 stable cell line was purchased from Integral Molecular company (Integral Cat #C-HA102). The cell line was maintained in DMEM with 10% FBS, 1×penicillin-streptomycin and 0.5 μg/ml puromycin.

### Molecular Cloning

H11-D4, MR17-K99Y and SR4 fragment genes were ordered from Genewiz. Human ACE2 gene was ordered from Addgene #1786.

pBAD-H11D4: primers H11D4-F and H11D4-R were used to amplify H11D4 fragments. pBAD vector and H11D4 fragment were joined with recombination cloning to create the pBAD-H11D4. Using pBAD-H11D4 as template, primers H11D4-R²⁷TAG-F and H11D4-R27TAG-R were used to generate pBAD-H11D4 (27TAG); primers H11D4-S30TAG-F and H11D4-S30TAG-R were used to generate pBAD-H11D4 (30TAG); primers H11D4-E100TAG-F and H11D4-E100TAG-R were used to generate pBAD-H11D4 (100TAG); primers H11D4-W112TAG-F and H11D4-W112TAG-R were used to generate pBAD-H11D4 (112TAG); primers H11D4-D115TAG-F and H11D4-D115TAG-R were used to generate pBAD-H11D4 (115TAG); primers H11D4-Y116TAG-F and H11D4-Y116TAG-R were used to generate pBAD-H11D4 (116TAG). Amino acid sequence of cpsGFP with Tyr66 highlighted in bold underline is shown below.

pBAD-MR17K99Y: MR17K99Y-F and MR17K99Y-R were used to amplify MR17K99Y fragments. pBAD vector and MR17K99Y fragment were joined with recombination cloning to create the pBAD-MR17K99Y. Using pBAD-MR17K99Y as template, primers MR17K99Y-Y99TAG-F and MR17K99Y-Y99TAG-R were used to generate pBAD-MR17K99Y (99TAG); primers MR17K99Y-D101TAG-F and MR17K99Y-D101TAG-R were used to generate pBAD-MR17K99Y (101TAG).

pBAD-SR4: SR4-F and SR4-R were used to amplify SR4 fragments. pBAD vector and SR4 fragment were joined with recombination cloning to create the pBAD-SR4. Using pBAD-SR4 as template, primers SR4-Y37TAG-F and SR4-Y37TAG-R were used to generate pBAD-SR4 (37TAG); primers SR4-H54TAG-F and SR4-H54TAG-R were used to generate pBAD-SR4 (54TAG); primers SR4-S57TAG-F and SR4-S57TAG-R were used to generate pBAD-SR4 (57TAG).

pcDNA3.1-ACE2: pcDNA-ACE2-Hind3-F and pcDNA-ACE2-His-BamHI-R were used to amplify the ACE2 gene. pcDNA3.1 vector and ACE2 fragment were joined with recombination cloning to create the pcDNA3.1-ACE2. Using pcDNA3.1-ACE2 as template, primers with TAG were used to generate its TAG mutants.

TABLE 3
Primers

| | |
|---|---|
| H11D4-F | TAAGAAGGAGATATACATAT |
| | GAAATATCTGCTGCCAACCG |
| | (SEQ ID NO: 383) |
| H11D4-R | GCCAAAACAGCCAAGCTTTT |
| | AATGATGGTGATGGTGGTGT |
| | T |
| | (SEQ ID NO: 384) |
| H11D4- | GGTTAGCGGTTAGACCTTTA |
| R27TAG-F | GCACC |
| | (SEQ ID NO: 385) |
| H11D4- | GCGCAACTCAGACGCAGA |
| R27TAG-R | (SEQ ID NO: 386) |
| H11D4- | TCGCACCTTTTAGACCGCCG |
| S30TAG-F | CGATGGGTTG |
| | (SEQ ID NO: 387) |
| H11D4- | CCGCTAACCGCGCAACTC |
| S30TAG-R | (SEQ ID NO: 388) |
| H11D4- | CGCGCGCACCTAGAACGTTC |
| E100TAG-F | GTA |
| | (SEQ ID NO: 389) |
| H11D4- | CAATAGTACACGGCGGTG |
| E100TAG-R | (SEQ ID NO: 390) |
| H11D4- | TTACGCCACGTAGCCGTACG |
| W112TAG-F | ATT |
| | (SEQ ID NO: 391) |
| H11D4- | TCGCTCAGCAGACTACGAAC |
| W112TAG-R | (SEQ ID NO: 392) |
| H11D4- | GTGGCCGTACTAGTACTGGG |
| D115TAG-F | GTC |
| | (SEQ ID NO: 393) |
| H11D4- | GTGGCGTAATCGCTCAGC |
| D115TAG-R | (SEQ ID NO: 394) |
| H11D4- | GCCGTACGATTAGTGGGGTC |
| Y116TAG-F | AAG |
| | (SEQ ID NO: 395) |
| H11D4- | CACGTGGCGTAATCGCTC |
| Y116TAG-R | (SEQ ID NO: 396) |
| MR17K99Y-F | CTTTAAGAAGGAGATATACA |
| | TATGAAATATCTGCTGCCAA |
| | CCG |
| | (SEQ ID NO: 397) |
| MR17K99Y-R | TCCGCCAAAACAGCCAAGCT |
| | TTTAATGGTGATGATGATGG |
| | (SEQ ID NO: 398) |
| MR17K99Y- | CTGCAACGTGTAGGATGATG |
| Y99TAG-F | GCC |
| | (SEQ ID NO: 399) |
| MR17K99Y- | TAGTACACGGCCGTATCC |
| Y99TAG-R | (SEQ ID NO: 400) |
| MR17K99Y- | CGTGTACGATTAGGGCCAGC |
| D101TAG-F | TGG |
| | (SEQ ID NO: 401) |
| MR17K99Y- | TTGCAGTAGTACACGGCC |
| D101TAG-R | (SEQ ID NO: 402) |
| SR4-F | CTTTAAGAAGGAGATATACA |
| | TATGAAATATCTGCTGCCAA |
| | CCGC |
| | (SEQ ID NO: 403) |
| SR4-R | ATCCGCCAAAACAGCCAAGC |
| | TTTTAATGATGATGGTGATG |
| | G |
| | (SEQ ID NO: 404) |
| SR4-Y37TAG-F | CATGTGGTGGTAGCGCCAAG |
| | CCC |
| | (SEQ ID NO: 405) |
| SR4-Y37TAG- | TTCCAGCTGTACACTGGAAA |
| R | GCC |
| | (SEQ ID NO: 406) |
| SR4-H54TAG-F | GATCGAAAGCTAGGGCGATA |
| | GCACCC |
| | (SEQ ID NO: 407) |
| SR4-H54TAG- | GCCGCAACCCATTCGCGT |
| R | (SEQ ID NO: 408) |
| SR4-S57TAG-F | CCACGGCGATTAGACCCGCT |
| | ACGCG |
| | (SEQ ID NO: 409) |
| SR4-S57TAG-R | CTTTCGATCGCCGCAACC |
| | (SEQ ID NO: 410) |
| pcDNA-ACE2- | ctagcgtttaaacttaagct |
| Hind3-F | tGCCACCatgtcaagctctt |
| | cctggctc |
| | (SEQ ID NO: 411) |
| pcDNA-ACE2- | cacactggactagtggatcc |
| His-BamHI-R | TTAGTGATGGTGATGATGAT |
| | Gggaaacagggggctgg |
| | (SEQ ID NO: 412) |
| ACE2- | ccaagacatttttgTAGaag |
| D30TAG-F | tttaaccacg |
| | (SEQ ID NO: 413) |
| ACE2- | CTAcaaaaatgtcttggcct |
| D30TAG-R | gttcctc |
| | (SEQ ID NO: 414) |
| ACE2- | gtttaaccacgaagccgaa |
| D38TAG-F | TAGctgttctatcaaag |
| | (SEQ ID NO: 415) |
| ACE2- | CTAttcggcttcgtggttaa |
| D38TAG-R | acttg |
| | (SEQ ID NO: 416) |
| ACE2- | caagtttaaccacgaagcc |
| E37TAG-F | TAGgacctgttctatcaaag |
| | (SEQ ID NO: 417) |
| ACE2- | CTAggcttcgtggttaaact |
| E37TAG-R | tgtc |
| | (SEQ ID NO: 418) |
| ACE2- | catttttggacaagtttaac |
| H34TAG-F | TAGgaagccgaagacctg |
| | (SEQ ID NO: 419) |
| ACE2- | ggcttcCTAgttaaacttgt |
| H34TAG-R | ccaaaaatg |
| | (SEQ ID NO: 420) |
| ACE2- | cgaagacctgttctatTAGa |
| Q42TAG-F | gttcacttgcttc |
| | (SEQ ID NO: 421) |
| ACE2- | ctCTAatagaacaggtcttc |
| Q42TAG-R | ggcttcgtg |
| | (SEQ ID NO: 422) |
| SDM-ACE2- | TGCCCAAATGTAGCCACTAC |
| 83TAG-F | AAG |
| | (SEQ ID NO: 423) |
| SDM-ACE2- | AGTGTGGACTGTTCCTTTAA |
| 83TAG-R | AAAG |
| | (SEQ ID NO: 424) |

### Nanobody Expression and Purification

pBAD-H11D4, pBAD-MR17K99Y or pBAD-SR was transformed into E. coli BL21(DE3) for widetype protein expression. For FSY incorporation, pBAD-H11D4 (27TAG), pBAD-H11D4 (30TAG), pBAD-H11D4 (100TAG), pBAD-H11D4 (112TAG), pBAD-H11D4 (115TAG), pBAD-H11D4 (116TAG), pBAD-MR17K99Y (99TAG), pBAD-MR17K99Y (101TAG), pBAD-SR4 (37TAG), pBAD-SR4(54TAG), or pBAD-SR4 (57TAG) was co-transformed respectively with pEVOL-FSYRS into E. coli BL21(DE3) for FSY incorporated protein expression.

The expression of nanobody was performed as described. (ref) The E coil cells were grown at 37° C., 220 rpm to an OD 0.5, after which 0.2% L-arabinose was added. For FSY incorporation, 1 mM FSY was added to the growth medium. The expression was carried out at 18° C., 220 rpm for 18 h. Cells were harvested at 8000 g, 4° C. for 30 min. The cell pellet was suspended with lysis buffer (20 mM Tris-HCl, 20 mM imidazole, 200 mM NaCl, pH 7.5) supplemented with EDTA free protease inhibitor cocktail and 1 μg/mL DNase. The cells were lysised by sonification, after which the cell lysis solution was centrifuged at 25,000 g at 4° C. for 40 min. The supernatant was collected and purified with 500 μL Ni-NTA resin affinity resin. The resin was washed and eluted with elution buffer (20 mM Tris-HCl, 300 mM imidazole, 200 mM NaCl, pH 7.5). The eluted protein was concentrated and exchanged with buffer (20 mM Tris-HCl, 200 mM NaCl, pH 7.5). To yield pure protein, TALON® metal affinity resin was further applied. The purification procedure was same as described above. The eluted proteins were analyzed by running 12% Tris-glycine SDS-PAGE gel.

### ACE2 Expression and Purification

One day before transfection, 2×10⁶ cells were seeded with pre-warmed Freestyle 293 media. The cells were transfected with 25 ug pcDNA-ACE2-34TAG and 25 ug pMP-FSYRS plasmids according to manufacturer's protocol. The cells were incubated with 30 min and then 2 mM FSY was added into the culture media dropwise. The supernatant was collected after 4 days post-transfection. 20 mM imidazole and pre-equilibrated Ni-NTA resin were added into the supernatant, followed by incubation 4° C. on the rotator for 1 h. The resin was washed and eluted with elution buffer (20 mM Tris-HCl, 300 mM imidazole, 200 mM NaCl, pH 7.5). The eluted protein was concentrated and exchanged with PBS, pH 7.5.

Nanobody Crosslinking—Spike with Nanobody at 1:5 Ratio

0.5 μM spike protein was incubated with 0.1 μM widetype or FSY mutant nanobody in 10 μl PBS at 37° C. for overnight. 10 μl loading dye with 100 mM DTT was added to each reaction and boiled at 95° C. for 10 min. Western blot analysis was performed against mouse Fc for the Fc region of mouse IgG1 at the C-terminus.

SR(54FSY) and SR(57FSY) Dynamics—Spike with Nanobody at 10:1 Ratio

0.5 μM spike protein was incubated with 5 μM widetype or FSY mutant nanobody in 10 μl PBS at 37° C. for different time length. 10 μL loading dye with 100 mM DTT was added to each reaction and boiled at 95° C. for 10 min. Western blot analysis was performed as described above.

293T-ACE2 cellular surface ACE2 binding competition assay with nanobody

Various concentrations of SR4 or SR4(57FSY) (100 μM, 50 μM, 10 μM, 2.5 μM, 0.5 μM and 0.1 μM) were mixed with 10 nM spike in a final volume of 10 μl HBSS for overnight incubation at 37 C. The mix was diluted 50 times with HBSS and incubated at 37° C. for 1 h. 1.5×10⁵ cells were incubated with the 100 μL diluted mix for 1 h at 37 C incubator. The cells were washed twice with HBSS and labeled with mFc-FITC for 1 h at RT, after which the cells were washed and analyzed on flow cytometry.

### Pseudovirus Infectivity Assay

293T-ACE2 cells was trypsinized and centrifuged at 200 g for 5 min to get the cell pellet. 4×10⁴ cells were added into each well of 48-well plate containing different volume of pseudovirus. The cells were cultured at 37° C. for additional 72 h, after which the cells were harvested for fluorescence-activated cell sorting (FACS) analysis to analyze the proportion of GFP positive cells.

### Nanobody Neutralization on 293T-ACE2 Cells

Various concentrations of SR4 or SR4(57FSY) (4 nM, 40 nM, 400 nM, 4 μM, 40 μM) were incubated with 40 μl pseudovirus in medium without FBS at 37° C. for 2 h. The mix was diluted ten times with medium without FBS and incubated at 37° C. for 1 h. The diluted mix was subsequently transferred to each well of 48-well plate. The cells were suspended at 4×10⁶ cells/ml, and 4×10⁴ cells and 2% FBS were added into each well containing the pseudovirus-nanobody mix. The cells were cultured at 37° C. for additional 72 h, after which the cells were harvested for fluorescence-activated cell sorting (FACS) analysis to analyze the proportion of GFP positive cells.

### Preparation of Biotinylated SR4

Biotin tag was installed on SR4 nanobody using genetic code expansion and click chemistry for detection purpose (FIG. 30). Briefly, 4-Azido-L-phenylalanine (AzF) was incorporated to the 5 position of SR4 nanobody SR4 (5AzF) following literature procedure. Then 1 mg/mL SR4 (5AzF) was react with 0.5 mM DBCO-biotin (Sigma-Aldrich, #760706) in PBS (pH 7.4) at room temperature for 3 h to install biotin group via the azido group utilizing copper-free click chemistry. Then, the excess DBCO-biotin was removed by 10 Kda cut-off spin column. The biotin labeled SR4 was concentrated to 1.5 mg/mL and store at −80° C.

Binding constant (K_{D}) measurement between spike protein RBD and SR4 nanobody.

Binding constant (K_{D}) between spike protein RBD and SR4 nanobody was measured by biolayer interferometry (BLI) using Octet Red384 systems (ForteBio).

Biotinylated SR4 was firstly loaded to streptavidin (SA) sensor (ForteBio #18-5019) by incubating SA sensor in 200 nM biotinylated SR4 in Kinetic Buffer (0.005% (v/v) Tween 20 and 0.1% BSA in PBS, pH=7.4) at 25° C. The sensor was equilibrated (baseline step) in Kinetic Buffer for 120 s, after which the sensor was incubated with spike protein RBD (association step) for 120 s The concentrations for spike protein RBD are 0, 250, 500, 1000, and 2000 nM. Then the sensor was then moved into Kinetic Buffer (dissociation step) for 300 s. Data was fitted for a 1:1 stoichiometry and K_{D}, Kₒₙ, and K_{off} were calculated using the built in software.

It is understood that the examples, embodiments, and aspects described herein are for illustrative purposes only and that various modifications or changes in light thereof will be suggested to persons skilled in the art and are to be included within the spirit and scope of this application and appended claims. All publications, patents, and patent applications cited herein are hereby incorporated by reference herein their entirety and for all purposes.

### REFERENCES FOR EXAMPLE 1

1. Gerstberger et al, Nat. Rev. Genet. 15, 829-845 (2014).; 2. Castello et al, Trends Genet. 29, 318-327 (2013). 3. Nussbacher et al, Trends Neurosci. 38, 226-236 (2015). 4. Castello et al, Mol. Cell 60, 696-710 (2016). 5. Benhalevy et al, Nat. Methods 15, 1074-1082 (2018).; 6. Hentze et al, Nat. Rev. Mol. Cell Biol. 19, 327-341 (2018).; 7. Müller-Mcnicoll et al, Nat. Rev. Genet. 14, 275-287 (2013).; 8. Wagenmakers et al, Eur. J. Biochem. 112, 323-330 (1980).; 9. Saito et al, Acc. Chem. Res. 18, 134-141 (1985).; 10. Licatalosi et al, Nature 456, 464-469 (2008).; 11. Hafner, M. et al. Transcriptome-wide identification of RNA-binding protein and microRNA target sites by PAR-CLIP. Cell 141, 129-141 (2010).; 12. Konig et al, Nat. Struct. Mol. Biol. 17, 909-915 (2010).; 13. Baltz et al, Mol. Cell 46, 674-690 (2012).; 14. Castello et al, Cell 149, 1393-1406 (2012).; 15. Lee et al, Mol. Cell 69, 354-369 (2018).; 16. Sugimoto et al, Genome Biol. 13, R⁶⁷ (2012).; 17. Xiang, Z. et al. Adding an unnatural covalent bond to proteins through proximity-enhanced bioreactivity. Nat. Methods 10, 885-888 (2013).; 18. Wang, L. Genetically encoding new bioreactivity. N. Biotechnol. 38, 16-25 (2017).; 19. Coin et al, Cell 155, 1258-1269 (2013).; 20. Yang, B. et al. Spontaneous and specific chemical cross-linking in live cells to capture and identify protein interactions. Nat. Commun. 8, 2240 (2017).; 21. Li, Q. et al. Developing Covalent Protein Drugs via Proximity-Enabled Reactive Therapeutics. Cell 182, 85-97 (2020).; 22. Wang, N. et al. Genetically encoding fluorosulfate-1-tyrosine to react with lysine, histidine, and tyrosine via SuFEx in proteins in vivo. J. Am. Chem. Soc. 140, 4995-4999 (2018).; 23. Zhang et al, Cell Res. 28, 1198-1201 (2018).; 24. Abudayyeh et al, Science 353, aaf5573 (2016). 25. Cox, et al, Science 358, 1019-1027 (2017).; 26. Yang et al, Mol. Cell 76, 981-997 (2019).; 27. Liu et al, Cell 168, 121-134 (2017).; 28. Smargon et la, Mol. Cell 65, 618-630 (2017).; 29. Wilusz et al, Nat. Struct. Mol. Biol. 12, 1031-1306 (2005). 30. Bilusic et al, RNA Biol. 11, 641-654 (2014).; 31. Holmqvist et al, EMBO J. 35, 991-1011 (2016).; 32. Chao et al, EMBO J. 31, 4005-4019 (2012).; 33. Wang, W., Wang, L., Wu, J., Gong, Q. & Shi, Y. Hfq-bridged ternary complex is important for translation activation of rpoS by DsrA. Nucleic Acids Res. 41, 5938-5948 (2013).; 34. Peng et al, Proc. Natl. Acad Sci. U.S.A. 111, 17134-17139 (2014).; 35. Tree et al, Mol. Cell 55, 199-213 (2014). 36. Schu et al, EMBO J 34, 2557-2573 (2015).; 37. Hoppmann, C. & Wang, L. Proximity-enabled bioreactivity to generate covalent peptide inhibitors of p53-Mdm4. Chem. Commun. 52, 5140-5143 (2016).; 38. Liu et al, J. Am. Chem. Soc. 141, 9458-9462 (2019).; 39. Nachtergaele et al, Annu. Rev. Genet. 52, 349-372 (2018).; 40. Meyer et al, Cell 149, 1635-1646 (2012).; 41. Dominissini et al, Nature 485, 201-206 (2012).; 42. Linder et al, Nat. Methods 12, 767-772 (2015).; 43. Xu et al, J Biol. Chem. 290, 24902-24913 (2015).; 44. Meyer et al, Nat. Methods 16, 1275-1280 (2019).; 45. Tang et al, Nucleic Acids Res. 49, D134-D143 (2020).; 46. Hwang et al, Cell Rep. 15, 423-435 (2016).; 47. Kini et al, RNA 22, 61-74 (2016).; 48. Mackereth et al, Curr. Opin. Struct. Biol. 22, 287-296 (2012).; 49. Lunde et al, Nat. Rev. Mol. Cell Biol. 8, 479-490 (2007).

### REFERENCES FOR EXAMPLE 2

(1) Wang, L.; Schultz, P. G. Expanding the Genetic Code. Angewandte Chemie Int Ed 2004, 44 (1), 34-66. (2) Li, Q.; Chen, Q.; Klauser, P. C.; Li, M.; Zheng, F.; Wang, N.; Li, X.; Zhang, Q.; Fu, X.; Wang, Q.; Xu, Y.; Wang, L. Developing Covalent Protein Drugs via Proximity-Enabled Reactive Therapeutics. Cell 2020, 182 (1), 85-97.e16. (3) Berdan, V. Y.; Klauser, P. C.; Wang, L. Covalent Peptides and Proteins for Therapeutics. Bioorgan Med Chem 2021, 29, 115896. (4) Yang, B.; Tang, S.; Ma, C.; Li, S.-T.; Shao, G.-C.; Dang, B.; DeGrado, W. F.; Dong, M.-Q.; Wang, P. G.; Ding, S.; Wang, L. Spontaneous and Specific Chemical Cross-Linking in Live Cells to Capture and Identify Protein Interactions. Nat Commun 2017, 8 (1), 2240. (5) Wang, L. Genetically Encoding New Bioreactivity. New Biotechnol 2017, 38 (Pt A), 16-25. (6) Xiang, Z.; Ren, H.; Hu, Y. S.; Coin, I.; Wei, J.; Cang, H.; Wang, L. Adding an Unnatural Covalent Bond to Proteins through Proximity-Enhanced Bioreactivity. Nat Methods 2013, 10 (9), 885-888. (7) Furman, J. L.; Kang, M.; Choi, S.; Cao, Y.; Wold, E. D.; Sun, S. B.; Smider, V. V.; Schultz, P. G.; Kim, C. H. A Genetically Encoded Aza-Michael Acceptor for Covalent Cross-Linking of Protein-Receptor Complexes. J Am Chem Soc 2014, 136 (23), 8411-8417. (8) Chen, X.-H.; Xiang, Z.; Hu, Y. S.; Lacey, V. K.; Cang, H.; Wang, L. Genetically Encoding an Electrophilic Amino Acid for Protein Stapling and Covalent Binding to Native Receptors. Acs Chem Biol 2014, 9 (9), 1956-1961. (9) Xiang, Z.; Lacey, V. K.; Ren, H.; Xu, J.; Burban, D. J.; Jennings, P. A.; Wang, L. Proximity-Enabled Protein Crosslinking through Genetically Encoding Haloalkane Unnatural Amino Acids. Angewandte Chemie Int Ed 2014, 53 (8), 2190-2193. (10) Dong et al, Angewandte Chemie Int Ed 2014, 53 (36), 9430-9448. (11) Wang, N.; Yang, B.; Fu, C.; Zhu, H.; Zheng, F.; Kobayashi, T.; Liu, J.; Li, S.; Ma, C.; Wang, P. G.; Wang, Q.; Wang, L. Genetically Encoding Fluorosulfate-1-Tyrosine To React with Lysine, Histidine, and Tyrosine via SuFEx in Proteins in Vivo. J Am Chem Soc 2018, 140 (15), 4995-4999. (12) Liu, J.; Cao, L.; Klauser, P. C.; Cheng, R.; Berdan, V. Y.; Sun, W.; Wang, N.; Ghelichkhani, F.; Yu, B.; Rozovsky, S.; Wang, L. A Genetically Encoded Fluorosulfonyloxybenzoyl-l-lysine for Expansive Covalent Bonding of Proteins via SuFEx Chemistry. J Am Chem Soc 2021, 143 (27), 10341-10351. (13) Lacey, V. K.; Louie, G. V.; Noel, J. P.; Wang, L. Expanding the Library and Substrate Diversity of the Pyrrolysyl-tRNA Synthetase to Incorporate Unnatural Amino Acids Containing Conjugated Rings. Chembiochem 2013, 14 (16), 2100-2105. (14) Takimoto, J. K.; Dellas, N.; Noel, J. P.; Wang, L. Stereochemical Basis for Engineered Pyrrolysyl-TRNA Synthetase and the Efficient in Vivo Incorporation of Structurally Divergent Non-Native Amino Acids. Acs Chem Biol 2011, 6 (7), 733-743. (15) Eigenbrot et al, Proc National Acad Sci 2010, 107 (34), 15039-15044. (16) D'Huyvetter et al, Clin Cancer Res 2017, 23 (21), 6616-6628. (17) Cho et al, Nature 2003, 421 (6924), 756-760. (18) Schmitz et al, Structure 2013, 21 (7), 1214-1224. (19) Diwanji et al, Biorxiv 2021, 2021.05.03.442258.

### REFERENCES FOR EXAMPLE 4

1. Dongen et al, Oncol 12, 1379-1389 (2007). 2. Wei, W. et al. ImmunoPET: Concept, Design, and Applications. Chem Rev 120, 3787-3851 (2020). 3. Dijkers et al, J Nucl Med 50, 974-981 (2009). 4. Dijkers et al, Clin Pharmacol Ther 87, 586-592 (2010). 5. Löfblom et al, Febs Lett 584, 2670-2680 (2010). 6. Feldwisch et al, J Mol Biol 398, 232-247 (2010). 7. Kang, W. et al. Nanobody Conjugates for Targeted Cancer Therapy and Imaging. Technol Cancer Res T 20, 15330338211010116 (2021). 8. Vaneycken et al, Faseb J 25, 2433-2446 (2011). 9. Knowles et al, J Clin Oncol 30, 3884-3892 (2012). 10. Bauer, R. A. Covalent inhibitors in drug discovery: from accidental discoveries to avoided liabilities and designed therapies. Drug Discov Today 20, 1061-1073 (2015). 11. Chang et al, Angewandte Chemie Int Ed 59, 15161-15165 (2020). 12. Berdan, V. Y., Klauser, P. C. & Wang, L. Covalent peptides and proteins for therapeutics. Bioorgan Med Chem 29, 115896 (2021). 13. Wang et al, Curr Opin Chem Biol 66, 102106 (2022). 14. Cao, L. & Wang, L. New covalent bonding ability for proteins. Protein Sci (2021) doi:10.1002/pro.4228. 15. Li, Q. et al. Developing Covalent Protein Drugs via Proximity-Enabled Reactive Therapeutics. Cell 182, 85-97.e16 (2020). 16. Arteaga et al, Nat Rev Clin Oncol 9, 16-32 (2012). 17. Capala et al, Curr Opin Oncol 22, 559-566 (2010). 18. Wang et al, J Am Chem Soc 140, 4995-4999 (2018). 19. Liu et al, J Am Chem Soc 143, 10341-10351 (2021). 20. D'Huyvetter, M. et al. 131I-labeled Anti-HER2 Camelid sdAb as a Theranostic Tool in Cancer Treatment. Clin Cancer Res 23, 6616-6628 (2017).

### REFERENCES FOR EXAMPLE 7

1. Xiang et al, Nat Methods 2013, 10 (9), 885-8. 2. Wang et al, J Am Chem Soc 2018, 140 (15), 4995-4999. 3. Hanke et al, Nat Commun 2020, 11 (1), 4420. 4. L¹ et al, Potent synthetic nanobodies against SARS-CoV-2 and molecular basis for neutralization. bioRxiv 2020. 5. Lan et al, Nature 2020, 581 (7807), 215-220. 6. Nonaka et al, Emerg Infect Dis 2021, 27 (5). 7. L¹ et al, Cell 2020, 182 (5), 1284-1294 e9. 8. Nelson et al, BioRxiv 2021, DOI: https://doi.org/10.1101/2021.01.13.426558. 9. Tian et al, bioRxiv 2021, DOI: https://doi.org/10.1101/2021.02.14.431117. 10. Madhi et al, N Engl J Med 2021, DOI: 10.1056/NEJMoa2102214 11. Wang et al, Nature 2021, DOI: 10.1038/s41586-021-03398-2. 12. Liu et al, N Engl J Med 2021, DOI: 10.1056/NEJMc2102017. 13. Planas et al, Nat Med 2021, DOI: 10.1038/s41591-021-01318-5

What is claimed is:

1. A compound of Formula (I) or a stereoisomer thereof: 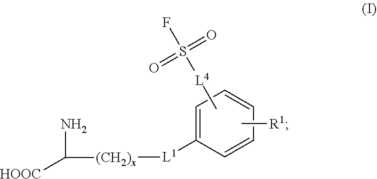
wherein:
L⁴ is a bond or —O—;
x is an integer from 1 to 8;
L¹ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1A}, SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl;
X¹ is independently —F, —Cl, —Br, or —I;
R^{1A} is hydrogen, substituted or unsubstituted alkyl, or substituted or unsubstituted heteroalkyl;
R^{1B} is hydrogen, substituted or unsubstituted alkyl, or substituted or unsubstituted heteroalkyl;
n1 is an integer from 0 to 4;
m1 is 1 or 2; and
v1 is 1 or 2.

2. The compound of claim 1, wherein -L⁴S(═O)₂F is para to the carbon atom linked to L¹.

3. The compound of claim 1, wherein -L⁴S(═O)₂F is meta to the carbon atom linked to L¹.

4. The compound of claim 1, wherein -L⁴S(═O)₂F is ortho to the carbon atom linked to L¹.

5. The compound of claim 1, wherein R₁ is para to -L⁴S(═O)₂F.

6. The compound of claim 1, wherein R¹ is meta to -L⁴S(═O)₂F.

7. The compound of claim 1, wherein R₁ is ortho to -L⁴S(═O)₂F.

8. The compound of claim 1, wherein the compound of Formula (I) is a compound of Formula (IA):

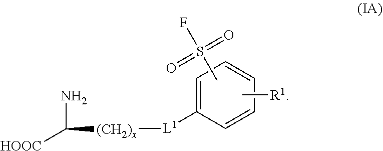

9. The compound of claim 8, wherein the compound of Formula (IA) is a compound of Formula (IB):

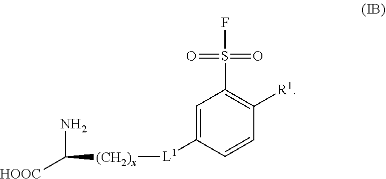

10. The compound of claim 1, wherein L⁴ is a bond.

11. The compound of a claim 1, wherein L⁴ is —O—.

12. The compound of claim 1, wherein x is an integer from 1 to 4.

13. The compound of claim 1, wherein L¹ is a bond.

14. The compound of claim 1, wherein L¹ is substituted or unsubstituted 2 to 6 membered heteroalkylene.

15. The compound of claim 1, wherein L¹ is —NH—C(O)—(CH₂)_{y}— or —NH—C(O)—O—(CH₂)_{y}—, and y is an integer from 0 to 2.

16. The compound of claim 1, wherein R¹ is substituted or unsubstituted heteroalkyl.

17. The compound of claim 1, wherein R¹ is unsubstituted 2 to 8 membered heteroalkyl.

18. The compound of claim 1, wherein R¹ is —O—(CH₂)ₘCH₃, and m is an integer from 0 to 4.

19. The compound of claim 1, wherein R¹ is hydrogen.

20. The compound of claim 1, wherein the compound of Formula (I) is a compound of Formula (IC) or a stereoisomer thereof:

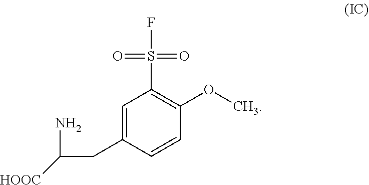 [SMILES: COc1ccc(CC(N)C(=O)O)cc1S(=O)(=O)F]

21. A compound of Formula (IV): 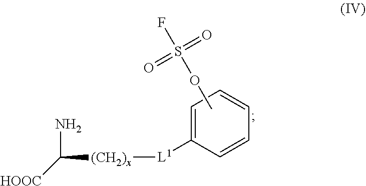
wherein: —OS(═O)₂F is meta or ortho to the carbon atom linked to L¹; x is an integer from 1 to 8; and L¹ is a bond, substituted or unsubstituted alkylene, or substituted or unsubstituted heteroalkylene.

22. The compound of claim 21, wherein x is an integer from 1 to 4.

23. The compound of claim 21, wherein L¹ is a bond.

24. The compound of claim 21, wherein L¹ is substituted or unsubstituted 2 to 6 membered heteroalkylene.

25. The compound of claim 21, wherein L¹ is —NH—C(O)—(CH₂)_{y}— or —NH—C(O)—O—(CH₂)_{y}—, and y is an integer from 0 to 2.

26. The compound of claim 21, wherein —OS(═O)₂F is ortho to the carbon atom linked to L¹.

27. The compound of claim 21, wherein —OS(═O)₂F is meta to the carbon atom linked to L¹.

28. The compound of claim 21, wherein the compound of Formula (IV) is a compound of Formula (IVA):

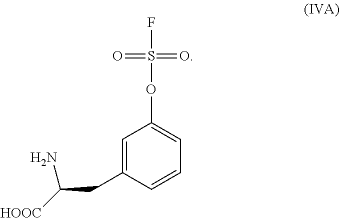 [SMILES: N[C@@H](Cc1cccc(OS(=O)(=O)F)c1)C(=O)O]

29. The compound of claim 21, wherein the compound of Formula (IV) is a compound of Formula (IVB):

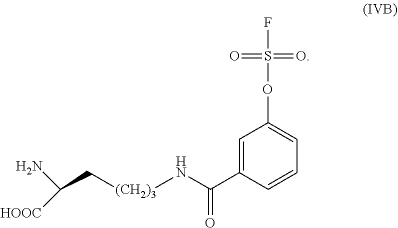

30. A protein comprising an unnatural amino acid, wherein the unnatural amino comprises a side chain of Formula (V): 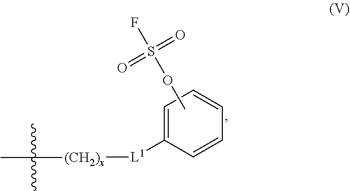
wherein: —OS(═O)₂F is meta or ortho to the carbon atom linked to L¹; x is an integer from 1 to 8; and L¹ is a bond, substituted or unsubstituted alkylene, or substituted or unsubstituted heteroalkylene.

31. The protein of claim 30, wherein x is an integer from 1 to 4.

32. The protein of claim 30, wherein L¹ is a bond.

33. The protein of claim 30, wherein L¹ is substituted or unsubstituted 2 to 6 membered heteroalkylene.

34. The protein of claim 30, wherein L¹ is —NH—C(O)—(CH₂)_{y}— or —NH—C(O)—O—(CH₂)_{y}—, and y is an integer from 0 to 2.

35. The protein of claim 30, wherein —OS(═O)₂F is ortho to the carbon atom linked to L¹.

36. The protein of claim 30, wherein —OS(═O)₂F is meta to the carbon atom linked to L¹.

37. The protein of claim 30, wherein the compound of Formula (V) is a compound of Formula (VA):

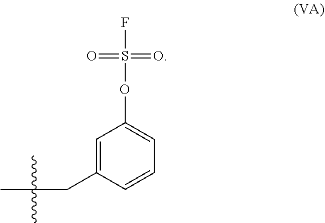 [SMILES: *Cc1cccc(OS(=O)(=O)F)c1]

38. The protein of claim 30, wherein the compound of Formula (V) is a compound of Formula (VB):

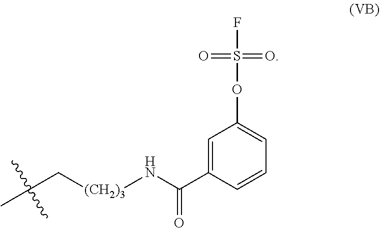

39. The protein of claim 30, wherein the protein is an antibody or an antibody variant.

40. The protein of claim 39, wherein the antibody variant is a single-chain variable fragment, a single-domain antibody, an affibody, or an antigen-binding fragment.

41. The protein of claim 30, wherein the protein is a receptor protein.

42. A nucleic acid encoding the protein of claim 30.

43. A vector comprising a nucleic acid of claim 42.

44. A biomolecule conjugate of Formula (VI): 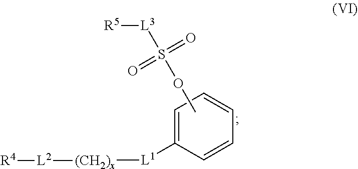
wherein:
—OS(═O)₂L³R⁵ is meta or ortho to the carbon atom linked to L;
R⁴ and R⁵ are each independently a peptidyl moiety, a carbohydrate moiety, or a nucleic acid moiety;
L¹ is a bond, substituted or unsubstituted alkylene, or substituted or unsubstituted heteroalkylene;
x is an integer from 1 to 8;
L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

45. The biomolecule conjugate of claim 44, wherein x is an integer from 1 to 4.

46. The biomolecule conjugate of claim 44, wherein L¹ is a bond.

47. The biomolecule conjugate of claim 44, wherein L¹ is substituted or unsubstituted 2 to 6 membered heteroalkylene.

48. The biomolecule conjugate of claim 44, wherein L¹ is —NH—C(O)—(CH₂)_{y}— or —NH—C(O)—O—(CH₂)_{y}—, and y is an integer from 0 to 2.

49. The biomolecule conjugate of claim 44, wherein —OS(═O)₂L³R⁵ is ortho to the carbon atom linked to L¹.

50. The biomolecule conjugate of claim 44, wherein —OS(═O)₂L³R⁵ is meta to the carbon atom linked to L¹.

51. The biomolecule conjugate of claim 44 having Formula (VIA):

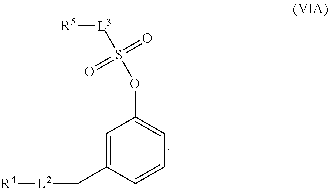

52. The biomolecule conjugate of claim 44 having Formula (VIB):

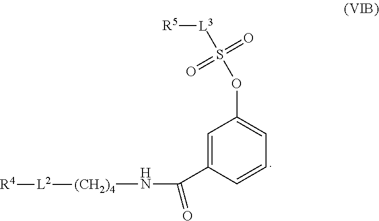

53. The biomolecule conjugate of claim 44, wherein R⁴ and R⁵ are each independently a peptidyl moiety.

54. The biomolecule conjugate of claim 44, wherein R⁵ is a peptidyl moiety comprising a lysine, histidine, or tyrosine bonded to L³.

55. The biomolecule conjugate of claim 44, wherein L³ is a bond.

56. The biomolecule conjugate of claim 44, wherein L² is a bond.

57. The biomolecule conjugate of claim 44, wherein the peptidyl moiety of R⁴ comprises an antibody or an antibody variant; and the peptidyl moiety of R⁵ comprises a receptor protein.

58. The biomolecule conjugate of claim 44, wherein the peptidyl moiety of R⁴ comprises a receptor protein and the peptidyl moiety of R⁵ comprises an antibody or an antibody variant.

59. The biomolecule conjugate of claim 57, wherein the antibody variant is a single-chain variable fragment, a single-domain antibody, an affibody, or an antigen-binding fragment.

60. A complex comprising a pyrrolysyl-tRNA synthetase comprising an amino acid sequence of SEQ ID NO:49, 56, 57, or 58 and the compound of claim 1.

61. The complex of claim 60, further comprising a tRNA^{Pyl}.

62. A cell comprising: (i) the compound of any one of claims 1 to 29; (ii) the protein of any one of claims 30 to 41; (iii) the nucleic acid of claim 42; (iv) the vector of claim 43; (v) the biomolecule conjugate of any one of claims 44 to 59; or (vi) the complex of claim 60 or 61.

63. The cell of claim 62, wherein the cell is a bacterial cell or a mammalian cell.

64. A compound of Formula (VII) or a stereoisomer thereof: 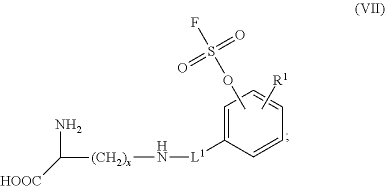
wherein: x is an integer from 1 to 8; L¹ is a bond, substituted or unsubstituted alkylene, or substituted or unsubstituted heteroalkylene; R¹ is halogen, —CX ¹³, —CHX¹₂, —CH₂X¹, —OCX ¹³, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted aryl, or substituted or unsubstituted heteroaryl.

65. The compound of claim 64, wherein —OS(═O)₂F is ortho to the carbon atom linked to L¹.

66. The compound of claim 64, wherein —OS(═O)₂F is meta to the carbon atom linked to L¹.

67. The compound of claim 64, wherein —OS(═O)₂F is para to the carbon atom linked to L¹.

68. The compound of claim 64, wherein R¹ is ortho to —OS(═O)₂F.

69. The compound of claim 64, wherein R¹ is meta to —OS(═O)₂F.

70. The compound of claim 64, wherein R¹ is para to —OS(═O)₂F.

71. The compound of claim 64, wherein the compound of Formula (VII) is a compound of Formula (VIIA):

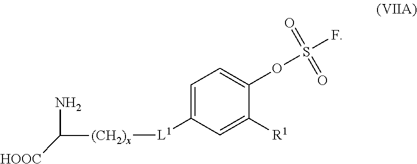

72. The compound of claim 64, wherein x is an integer from 1 to 4.

73. The compound of claim 64, wherein L¹ is a bond.

74. The compound of claim 64, wherein the compound of Formula (VII) is a compound of Formula (VIIB):

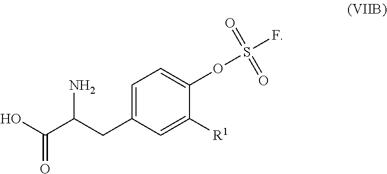

75. The compound of claim 64, wherein R¹ is halogen, —CX¹₃, —CHX¹₂, —CH₂X¹, —CN, —SOₙ₁R^{1A}, SOᵥ₁NR^{1A}R^{1B}, —N(O)ₘ₁, —C(O)R^{1A}, —C(O)—OR^{1A}, —C(O)NR^{1A}R^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

76. The compound of claim 75, wherein R¹ is —CX¹₃, —CHX¹₂, —CH₂X¹, —CN, —SOₙ₁₁R^{1A}, —SOᵥ₁NR^{1A}R^{1B}, —N(O)ₘ₁, —C(O)R^{1A}, —C(O)—OR^{1A}, or —C(O)NR^{1A}R^{1B}.

77. The compound of claim 75, wherein R^{1A} and R^{1B} are hydrogen.

78. The compound of claim 75, wherein R¹ is halogen.

79. The compound of claim 64, wherein the compound of Formula (VII) is a compound of Formula (VIID) or a stereoisomer thereof:

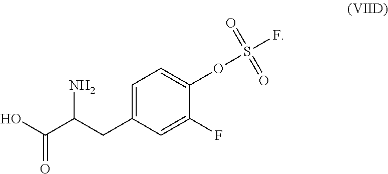 [SMILES: NC(Cc1ccc(OS(=O)(=O)F)c(F)c1)C(=O)O]

80. A protein comprising an unnatural amino acid, wherein the unnatural amino comprises a side chain of Formula (VIII): 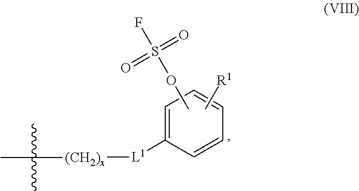
wherein: x is an integer from 1 to 8; L¹ is a bond, substituted or unsubstituted alkylene, or substituted or unsubstituted heteroalkylene; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted aryl, or substituted or unsubstituted heteroaryl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2.

81. The protein of claim 80, wherein —OS(═O)₂F is ortho to the carbon atom linked to L¹.

82. The protein of claim 80, wherein —OS(═O)₂F is meta to the carbon atom linked to L¹.

83. The protein of claim 80, wherein —OS(═O)₂F is para to the carbon atom linked to L¹.

84. The protein of claim 80, wherein R¹ is ortho to —OS(═O)₂F.

85. The protein of claim 80, wherein R¹ is meta to —OS(═O)₂F.

86. The protein of claim 80, wherein R¹ is para to —OS(═O)₂F.

87. The protein of claim 80, wherein the side chain of Formula (VIII) is a side chain of Formula (VIIIA):

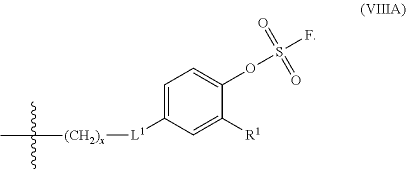

88. The protein of claim 80, wherein x is an integer from 1 to 4.

89. The protein of claim 80, wherein L¹ is a bond.

90. The protein of claim 80, wherein the side chain of Formula (VIII) is a side chain of Formula (VIIIB):

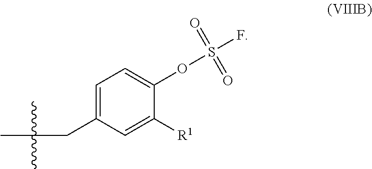

91. The protein of claim 80, wherein R¹ is halogen, —CX¹₃, —CHX¹₂, —CH₂X¹, —CN, —SOₙ₁R^{1A}, SOᵥ₁NR^{1A}R^{1B}, —N(O)ₘ₁, —C(O)R^{1A}, —C(O)OR^{1A}, —C(O)NR^{1A}R^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

92. The protein of claim 91, wherein R¹ is —CX¹₃, —CHX¹₂, —CH₂X¹, —CN, —SOₙ₁R^{1A}, SOᵥ₁NR^{1A}R^{1B}, —N(O)ₘ₁, —C(O)R^{1A}, —C(O)—OR^{1A}, or —C(O)NR^{1A}R^{1B}.

93. The protein of claim 91, wherein R^{1A} and R^{1B} are hydrogen.

94. The protein of claim 91, wherein R¹ is halogen.

95. The protein of claim 94, wherein R¹ is —F.

96. The protein of claim 80, wherein the protein is an antibody or an antibody variant.

97. The protein of claim 80, wherein the protein is an antigen-binding fragment, a single-chain variable fragment, a single-domain antibody, or an affibody.

98. The protein of claim 80, wherein the protein is a receptor protein.

99. A biomolecule conjugate comprising a first biomolecule moiety conjugated to a second biomolecule moiety through a bioconjugate linker, wherein the bioconjugate linker is Formula (X): 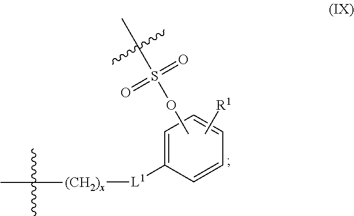
wherein: x is an integer from 1 to 8; L¹ is a bond, substituted or unsubstituted alkylene, or substituted or unsubstituted heteroalkylene; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, substituted or unsubstituted heteroalkyl, substituted or unsubstituted aryl, or substituted heteroaryl.

100. The biomolecule conjugate of claim 99 having Formula (IXA): 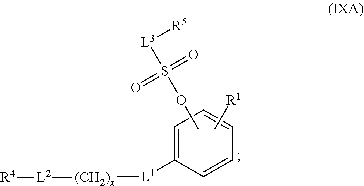
wherein: R² is the first biomolecule; R³ is the second biomolecule; L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{1A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

101. The biomolecule conjugate of claim 100, wherein —OS(═O)₂F is ortho to the carbon atom linked to L¹.

102. The biomolecule conjugate of claim 100, wherein —OS(═O)₂F is meta to the carbon atom linked to L¹.

103. The biomolecule conjugate of claim 100, wherein —OS(═O)₂F is para to the carbon atom linked to L¹.

104. The biomolecule conjugate of claim 100, wherein R¹ is ortho to —OS(═O)₂F.

105. The biomolecule conjugate of claim 100, wherein R¹ is meta to —OS(═O)₂F.

106. The biomolecule conjugate of claim 100, wherein R¹ is para to —OS(═O)₂F.

107. The biomolecule conjugate of claim 100, wherein Formula (IXA) is a compound of Formula (XB):

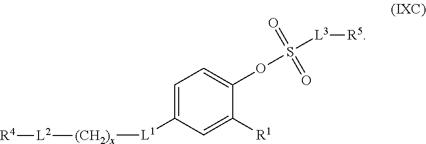

108. The biomolecule conjugate of claim 100, wherein x is an integer from 1 to 4.

109. The biomolecule conjugate of claim 100, wherein L¹ is a bond.

110. The biomolecule conjugate of claim 100, wherein L¹ is substituted or unsubstituted 2 to 6 membered heteroalkylene.

111. The biomolecule conjugate of claim 100, wherein L¹ is —NH—C(O)—(CH₂)_{y}— or —NH—C(O)—O—(CH₂)_{y}—, and y is an integer from 0 to 2.

112. The biomolecule conjugate of claim 100, wherein: L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-, substituted or unsubstituted heteroarylene; L¹² is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl; L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹²-substituted or unsubstituted heteroarylene; and L¹³ is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N3, unsubstituted alkyl, unsubstituted heteroalkyl, unsubstituted cycloalkyl, unsubstituted heterocycloalkyl, unsubstituted aryl, or unsubstituted heteroaryl.

113. The biomolecule conjugate of claim 100, wherein L³ is a bond.

114. The biomolecule conjugate of claim 100, wherein L² is a bond.

115. The biomolecule conjugate of claim 100, wherein the biomolecule conjugate of Formula (IXA) is a biomolecule conjugate of Formula (IXE), Formula (IXF), or Formula (IXG):

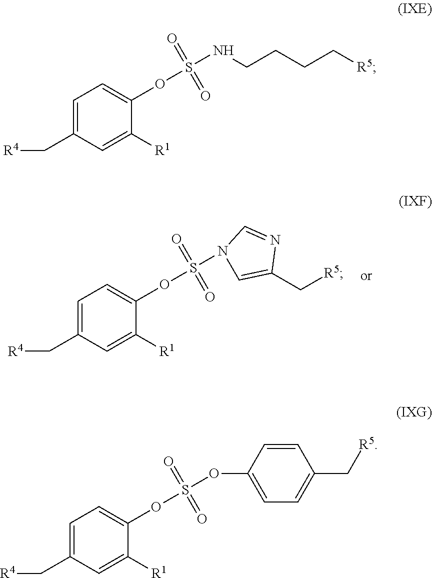

116. The biomolecule conjugate of claim 100, wherein R¹ is halogen, —CX¹₃, —CHX¹₂, —CH₂X¹, —CN, —SOₙ₁R^{1A}, —SOᵥ₁NR^{1A}R^{1B}, —N(O)ₘ₁, —C(O)R^{1A}, —C(O)—OR^{1A}, —C(O)NR^{1A}R^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

117. The biomolecule conjugate of claim 116, wherein R¹ is —CX¹₃, —CHX¹₂, —CH₂X¹, —CN, —SOₙ₁R^{1A}, —SOᵥ₁NR^{1A}R^{1B}, —N(O)ₘ₁, —C(O)R^{1A}, —C(O)—OR^{1A}, or —C(O)NR^{1A}R^{1B}.

118. The biomolecule conjugate of claim 116, wherein R^{1A} and R^{1B} are hydrogen.

119. The biomolecule conjugate of claim 100, wherein R¹ is halogen.

120. The biomolecule conjugate of claim 119, wherein R¹ is —F.

121. The biomolecule conjugate of claim 100, wherein R⁴ and R⁵ are each independently a peptidyl moiety.

122. The biomolecule conjugate of claim 121, wherein the peptidyl moiety of R⁴ comprises an antibody or an antibody variant; and the peptidyl moiety of R⁵ comprises a receptor protein.

123. The biomolecule conjugate of claim 121, wherein the peptidyl moiety of R⁴ comprises a receptor protein and the peptidyl moiety of R⁵ comprises an antibody or an antibody variant.

124. The biomolecule conjugate of claim 122, wherein the antibody variant is an antigen-binding fragment, a single-chain variable fragment, a single-domain antibody, or an affibody.

125. The biomolecule conjugate of claim 122, wherein the receptor protein is a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SiP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor.

126. The biomolecule conjugate of claim 122, wherein the receptor protein is a G protein-coupled receptor.

127. A complex comprising a pyrrolysyl-tRNA synthetase and the compound of any one of claims 64 to 79.

128. The complex of claim 127, wherein the pyrrolysyl-tRNA synthetase has an amino acid sequence with at least 90% sequence identity to SEQ ID NO:49, 56, 57, or 58.

129. The complex of claim 128, wherein the pyrrolysyl-tRNA synthetase has an amino acid sequence as set forth in SEQ ID NO:49, 56, 57, or 58.

130. The complex of claim 127, further comprising a tRNA^{Pyl}.

131. The complex of claim 130, wherein the tRNA^{Pyl} has the sequence as set forth in SEQ ID NO:15.

132. A cell comprising (i) the compound of any one of claims 64 to 79; (ii) the protein of any one of claims 80 to 98; (iii) the biomolecule conjugate of any one of claims 99 to 126; or (vi) the complex of any one of claims 127 to 131.

133. The cell of claim 132, wherein the cell is a bacterial cell or a mammalian cell.

134. A pyrrolysyl-tRNA synthetase comprising an amino acid sequence of SEQ ID NO:49, 56, 57, or 58.

135. A nucleic acid encoding the pyrrolysyl-tRNA synthetase of claim 134.

136. A vector comprising a nucleic acid encoding the pyrrolysyl-tRNA synthetase of claim 134.

137. A nanobody comprising an unnatural amino acid within CDR1, CDR2, or CDR3 of the nanobody; wherein the unnatural amino acid comprises a side chain of Formula (II): 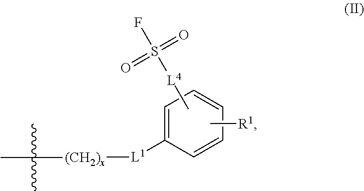
wherein: L⁴ is a bond or —O—; x is an integer from 1 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

138. The nanobody of claim 137, wherein the unnatural amino acid comprises:
(a) a side chain of Formula (IE-A): 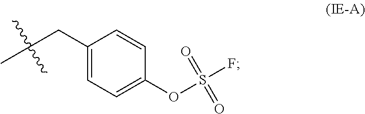 [SMILES: *Cc1ccc(OS(=O)(=O)F)cc1]
(b) a side chain of Formula (VA): 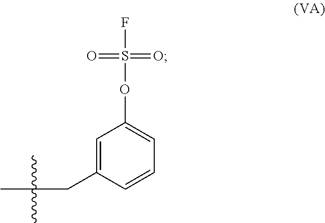 [SMILES: *Cc1cccc(OS(=O)(=O)F)c1]
(c) a side chain of Formula (VIIIC): 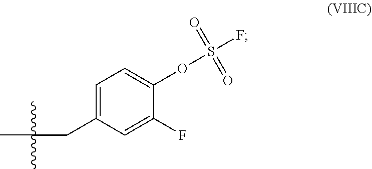 [SMILES: *Cc1ccc(OS(=O)(=O)F)c(F)c1]
(d) a side chain of Formula (VB): 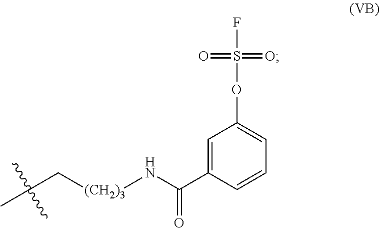
(e) a side chain of Formula (VC):

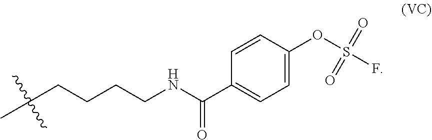 [SMILES: *CCCCNC(=O)c1ccc(OS(=O)(=O)F)cc1]

139. The nanobody of claim 137, wherein the nanobody comprises one unnatural amino acid.

140. The nanobody of claim 137, comprising CDR1 as set forth in SEQ ID NO:67, CDR2 as set forth in SEQ ID NO:68; and CDR3 as set forth in SEQ ID NO:70.

141. The nanobody of claim 137, comprising CDR1 as set forth in SEQ ID NO:67, CDR2 as set forth in SEQ ID NO:68; and CDR3 as set forth in SEQ ID NO:71.

142. The nanobody of claim 137, comprising CDR1 as set forth in SEQ ID NO:61, CDR2 as set forth in SEQ ID NO:62; and CDR3 as set forth in SEQ ID NO:64, 200, 202, 204, 206, 208, 210, or 212.

143. The nanobody of claim 137, comprising CDR1 as set forth in SEQ ID NO:78, CDR2 as set forth in SEQ ID NO:76, and CDR3 as set forth in SEQ ID NO:77.

144. The nanobody of claim 137, comprising CDR1 as set forth in SEQ ID NO:81, CDR2 as set forth in SEQ ID NO:84 or SEQ ID NO:85; and CDR3 as set forth in SEQ ID NO:83.

145. The nanobody of claim 137, comprising CDR1 as set forth in SEQ ID NO: 86, CDR2 as set forth in SEQ ID NO:82; and CDR3 as set forth in SEQ ID NO:83.

146. The nanobody of claim 137, comprising CDR1 as set forth in SEQ ID NO:81, CDR2 as set forth in SEQ ID NO:87; and CDR3 as set forth in SEQ ID NO:83.

147. The nanobody of claim 137, comprising CDR1 as set forth in SEQ ID NO:93, CDR2 as set forth in any one of SEQ ID NOS:96-102 and 105-113; and CDR3 as set forth in SEQ ID NO:95.

148. The nanobody of claim 137, comprising CDR1 as set forth in SEQ ID NO:93, CDR2 as set forth in any one of SEQ ID NO:94; and CDR3 as set forth in any one of SEQ ID NOS:103, 104, 114, or 115.

149. The nanobody of claim 137, comprising CDR1 as set forth in SEQ ID NO: 155, CDR2 as set forth in any one of SEQ ID NO:156; and CDR3 as set forth in SEQ ID NO:181 or 182.

150. The nanobody of claim 137, comprising CDR1 as set forth in SEQ ID NO:218, 219, 220, 221, or 222, CDR2 as set forth in SEQ ID NO:216, or CDR3 as set forth in SEQ ID NO:217.

151. The nanobody of claim 137, comprising CDR1 as set forth in SEQ ID NO:215, CDR2 as set forth in SEQ ID NO:216, and CDR3 as set forth in SEQ ID NO:223, 224, 225, or 226.

152. The nanobody of claim 137, comprising CDR1 as set forth in SEQ ID NO:243, 244, 245, or 246, CDR2 as set forth in SEQ ID NO:241, and CDR3 as set forth in SEQ ID NO:242.

153. The nanobody of claim 137, comprising CDR1 as set forth in SEQ ID NO:240, CDR2 as set forth in SEQ ID NO:247, 248, 249, or 250, and CDR3 as set forth in SEQ ID NO:242.

154. The nanobody of claim 137, comprising CDR1 as set forth in SEQ ID NO:240, CDR2 as set forth in SEQ ID NO:241, and CDR3 as set forth in SEQ ID NO:251, 252, 253, or 254.

155. The nanobody of claim 137, comprising CDR1 as set forth in SEQ ID NO:31, CDR2 as set forth in SEQ ID NO:32; and CDR3 as set forth in SEQ ID NO:33; wherein the unnatural amino acid is at a position corresponding to position 5 or position 8 in SEQ ID NO:32.

156. The nanobody of claim 137, comprising CDR1 as set forth in SEQ ID NO:35, CDR2 as set forth in SEQ ID NO:36; and CDR3 as set forth in SEQ ID NO:37; wherein the unnatural amino acid is at a position corresponding to position 4 in SEQ ID NO:37.

157. The nanobody of claim 137, comprising CDR1 as set forth in SEQ ID NO:39, CDR2 as set forth in SEQ ID NO:40; and CDR3 as set forth in SEQ ID NO:41; wherein the unnatural amino acid is at a position corresponding to position 18 or position 19 in SEQ ID NO:41.

158. The nanobody of claim 137, wherein the nanobody has an amino acid sequence with at least 90% sequence identity to any one of SEQ ID NOS:65, 73, 79, 88, 89, 90, 91, 116-127, 183-189, 199, 201, 203, 205, 207, 209, 211, 227-238, and 255-267; provided that the nanobody has 100% sequence identity with CDR1, CDR2, and CDR3 therein.

159. The nanobody of claim 137, wherein the nanobody has an amino acid sequence as set forth in any one of SEQ ID NOS:65, 73, 79, 88, 89, 90, 91, 116-127, 183-189, 199, 201, 203, 205, 207, 209, 211, 227-238, and 255-267.

160. The nanobody of claim 137, provided that the nanobody is not nanobody 7D12; provided that the nanobody has less than 100% sequence identity with CDR1 as set forth in SEQ ID NO:155, CDR2 as set forth in SEQ ID NO:156, or CDR3 as set forth in SEQ ID NO:157; or provided that the nanobody having CDR1 as set forth in SEQ ID NO:155, CDR2 as set forth in SEQ ID NO:156, and CDR3 as set forth in SEQ ID NO: 157 does not contain an FSY unnatural amino acid in CDR1, CDR2, or CDR3 and does not contain an FSK unnatural amino acid in CDR1, CDR2, or CDR3

161. The nanobody of claim 137, provided that the nanobody is not nanobody KN035; provided that the nanobody has less than 100% sequence identity to CDR1, CDR2, and CDR3 in SEQ ID NO:177 or SEQ ID NO:178; or provided that the nanobody has less than 100% sequence identity to SEQ ID NO:177 or SEQ ID NO:178.

162. The nanobody of claim 137, further comprising a detectable agent.

163. The nanobody of claim 162, wherein the detectable agent is a radioisotope.

164. The nanobody of claim 163, wherein the radioisotope is ¹¹C, ¹³N, ¹⁵O, ¹⁸F, ⁶⁴Cu, ⁶⁸Ga, ⁷⁸Br, ⁸²Rb, ⁸⁶Y, ⁸⁹Zr, ⁹⁰Y, ²²Na, ²⁶Al, ⁴⁰K, ⁸³Sr, or ¹²⁴I, ²¹¹At, ²²⁷Th, ²²⁵Ac, ²²³Ra, ²¹³Bi, or ²¹²Bi.

165. The nanobody of claim 137, further comprising a therapeutic agent.

166. A fusion protein comprising a first protein and a second protein, wherein the first protein is a first nanobody of claim 137.

167. The fusion protein of claim 166, wherein the first protein is covalently bonded to the second protein via a glycine-serine peptide linker.

168. The fusion protein of claim 166, wherein the second protein is an antigen-binding fragment, a single-chain variable fragment, a second nanobody, an affibody,.

169. The fusion protein of claim 166, wherein the second protein has at least 90% sequence identity to the amino acid sequence of SEQ ID NO:219, SEQ ID NO:137, SEQ ID NO:138, SEQ ID NO:139, SEQ ID NO:180, SEQ ID NO:192, SEQ ID NO: 193, SEQ ID NO:194, SEQ ID NO:195, SEQ ID NO:196, SEQ ID NO:197, or SEQ ID NO:198.

170. A protein comprising an unnatural amino acid within CDR-L1, CDR-L2, CDR-L3, CDR-H1, CDR-H2, or CDR-H3, wherein the protein is an antigen-binding fragment, a single-chain variable fragment, or an antibody.

171. The protein of claim 170, wherein the unnatural amino acid comprises:
(a) a side chain of Formula (IE-A): 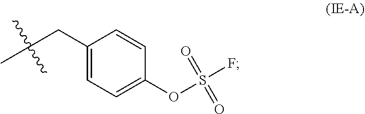 [SMILES: *Cc1ccc(OS(=O)(=O)F)cc1]
(b) a side chain of Formula (VA): 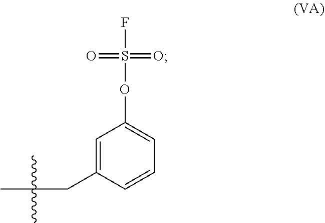 [SMILES: *Cc1cccc(OS(=O)(=O)F)c1]
(c) a side chain of Formula (VIIIC): 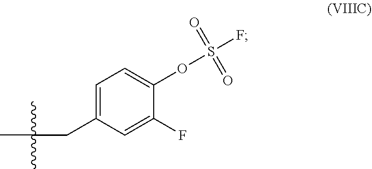 [SMILES: *Cc1ccc(OS(=O)(=O)F)c(F)c1]
(d) a side chain of Formula (VB): 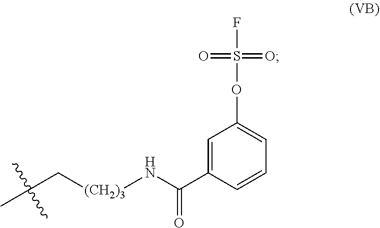
or
(e) a side chain of Formula (VC):

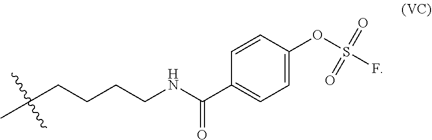 [SMILES: *CCCCNC(=O)c1ccc(OS(=O)(=O)F)cc1]

172. The protein of claim 170, wherein the protein is an antigen-binding fragment.

173. The protein of claim 172, wherein the antigen-binding fragment is a trastuzumab antigen-binding fragment having CDR-L1 as set forth in SEQ ID NO:163, CDR-L2 as set forth in SEQ ID NO:165, CDR-L3 as set forth in SEQ ID NO:165, CDR-H1 as set forth in SEQ ID NO:171, CDR-H2 as set forth in SEQ ID NO:172, and CDR-H3 as set forth in SEQ ID NO:173.

174. The protein of claim 172, wherein the protein is an antigen-binding fragment having CDR-L1 as set forth in SEQ ID NO:163, CDR-L2 as set forth in SEQ ID NO:165, CDR-L3 as set forth in SEQ ID NO:166 or 167, CDR-H1 as set forth in SEQ ID NO:171, CDR-H2 as set forth in SEQ ID NO: 172, and CDR-H3 as set forth in SEQ ID NO:173.

175. A protein having at least 90% sequence identity to any one of SEQ ID NOS:2, 3, 4, 22, 26, 29, 174, 176, 179, 180, 192, 193, 194, 195, 196, 197, 198, and 199, provided that the protein comprises the unnatural amino acid therein.

176. The protein of claim 170, further comprising a detectable agent.

177. The protein of claim 176, wherein the detectable agent is a radioisotope.

178. The protein of claim 170, further comprising a therapeutic agent.

179. A pharmaceutical composition comprising: (i) a pharmaceutically acceptable excipient, and (ii) the nanobody of any one of claims 137 to 165, the fusion protein of any one of claims 166 to 169, or the protein of any one of claims 170 to 178.

180. A method of detecting cancer in a patient in need thereof, the method comprising administering to the patient an effective amount of the nanobody of any one of claims 137 to 165, the fusion protein of any one of claims 166 to 169, or the protein of any one of claims 170 to 178, thereby detecting cancer in the patient.

181. A method of monitoring cancer progression or cancer treatment in a patient in need thereof, the method comprising administering to the patient an effective amount of the nanobody of any one of claims 137 to 165, the fusion protein of any one of claims 166 to 169, or the protein of any one of claims 170 to 178 at a first time point, thereby detecting cancer in the patient; and administering to the patient an effective amount of the nanobody of any one of claims 137 to 165, the fusion protein of any one of claims 166 to 169, or the protein of any one of claims 170 to 178, respectively, at a second time point later than the first time point, thereby monitoring the cancer progression or cancer treatment.

182. A recombinant protein comprising an ACE2 receptor protein having an unnatural amino acid side chain at a position corresponding to position 34, 37, or 42 in the ACE2 receptor protein; wherein the unnatural amino acid side chain is capable of covalently binding to a lysine, tyrosine, or histidine.

183. The recombinant protein of claim 182, wherein the unnatural amino acid side chain is a moiety of the Formula (IE-A):

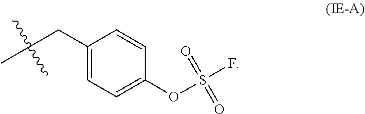 [SMILES: *Cc1ccc(OS(=O)(=O)F)cc1]

184. A RNA-binding protein comprising an unnatural amino acid, wherein the unnatural amino comprises a side chain of Formula (II): 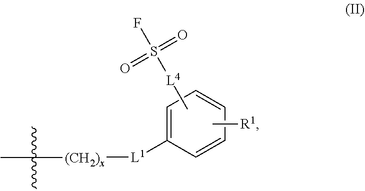
wherein: the RNA binding protein is a CRISPR protein or a RNA chaperone; L⁴ is a bond or —O—; x is an integer from 1 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

185. The RNA-binding protein of claim 184, wherein L⁴ is a bond.

186. The RNA-binding protein of claim 184, wherein L⁴ is —O—.

187. The RNA-binding protein of claim 184, wherein x is an integer from 1 to 4.

188. The RNA-binding protein of claim 184, wherein x is 1.

189. The RNA-binding protein of claim 184, wherein L¹ is a bond.

190. The RNA-binding protein of claim 184, wherein L¹ is substituted or unsubstituted 2 to 6 membered heteroalkylene.

191. The RNA-binding protein of claim 184, wherein L¹ is —NH—C(O)—(CH₂)_{y}— or —NH—C(O)—O—(CH₂)_{y}—, and y is an integer from 0 to 2.

192. The RNA-binding protein of claim 184, wherein R¹ is substituted or unsubstituted heteroalkyl.

193. The RNA-binding protein of claim 184, wherein R¹ is unsubstituted 2 to 8 membered heteroalkyl.

194. The RNA-binding protein of claim 184, wherein R′ is —O—(CH₂)ₘCH₃, and m is an integer from 0 to 4.

195. The RNA-binding protein of claim 184, wherein R¹ is ortho to —S(═O)₂F.

196. The RNA-binding protein of claim 184, wherein R¹ is hydrogen.

197. The RNA-binding protein of claim 184, wherein the side chain of Formula (II) has the structure of Formula (IIC):

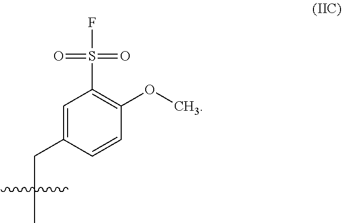 [SMILES: *Cc1ccc(OC)c(S(=O)(=O)F)c1]

198. The RNA-binding protein of claim 184, wherein the side chain of Formula (II) has the structure of Formula (IIE):

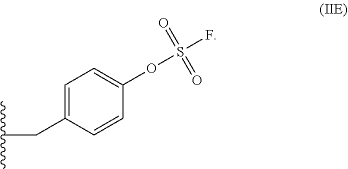 [SMILES: *Cc1ccc(OS(=O)(=O)F)cc1]

199. The RNA binding protein of claim 184, wherein the RNA binding protein is the CRISPR protein.

200. The RNA binding protein of claim 184, wherein the CRISPR protein comprises the unnatural amino acid sidechain at a position corresponding to position 133 or position 380, with reference to the amino acid sequence of catalytically inactive Cas13b protein from Prevotella sp. P5-125.

201. The RNA binding protein of claim 184, wherein the CRISPR protein comprises the unnatural amino acid sidechain at a position corresponding to position 128, position 133, position 380, position 1053, or position 1058, with reference to the amino acid sequence of catalytically inactive Cas13b protein from Prevotella sp. P5-125.

202. The RNA binding protein of claim 184, wherein the CRISPR protein is a catalytically inactive Cas13b protein.

203. The RNA binding protein of claim 202, wherein the catalytically inactive Cas13b protein is from Prevotella sp. P5-125, Bergeyella zoohelcum, or Prevotella buccae.

204. The RNA binding protein of claim 202, wherein the catalytically inactive Cas13b protein comprises the unnatural amino acid sidechain at a position corresponding to position 133 or position 380.

205. The RNA binding protein of 203, wherein the catalytically inactive Cas13b protein from Prevotella sp. P5-125 comprises the unnatural amino acid sidechain at a position corresponding to position R128, H133, R380, R1053, H1058, or two or more thereof; the catalytically inactive Cas13b protein from Bergeyella zoohelcum comprises the unnatural amino acid sidechain at a position corresponding to position R116, H121, R459, R1177, H1182, or two or more thereof; and the catalytically inactive Cas13b protein from Prevotella buccae comprises the unnatural amino acid sidechain at a position corresponding to position R156, H161, K393, R402, R1068, H1073, or two or more thereof.

206. The RNA binding protein of claim 184, wherein the CRISPR protein is a catalytically inactive Cas9 protein.

207. The RNA binding protein of claim 206, wherein the catalytically inactive Cas9 protein is from Streptococcus pyogenes, Staphylococcus aureus, or Actinomyces naeslundii.

208. The RNA binding protein of claim 207, wherein the catalytically inactive Cas9 protein from Streptococcus pyogenes comprises the unnatural amino acid sidechain at a position corresponding to position D10, E762, H983, D986, H840, N863, D839, or two or more thereof; the catalytically inactive Cas9 protein from Staphylococcus aureus comprises the unnatural amino acid sidechain at a position corresponding to position D10, E477, H701, D704, H557, N580, D556, or two or more thereof; and the catalytically inactive Cas9 protein from Actinomyces naeslundii comprises the unnatural amino acid sidechain at a position corresponding to position D17, E505, H736, D739, H582, N606, D581, or two or more thereof.

209. The RNA binding protein of claim 184, wherein the CRISPR protein is a catalytically inactive Cas12a protein.

210. The RNA binding protein of claim 209, wherein the catalytically inactive Cas12a protein is from Acidaminococcus sp. BV3L6, Lachnospiraceae bacterium ND2006, or Francisella novicida U112.

211. The RNA binding protein of claim 210, wherein the catalytically inactive Cas12a protein from Acidaminococcus sp. BV3L6 comprises the unnatural amino acid sidechain at a position corresponding to position D908, E993, D1263, R1226, D1235, or two or more thereof; the catalytically inactive Cas12a protein from Lachnospiraceae bacterium ND2006 comprises the unnatural amino acid sidechain at a position corresponding to position D833, E926, D1181, R1139, D1149, or two or more thereof, and the catalytically inactive Cas12a protein from Francisella novicida U112 comprises the unnatural amino acid sidechain at a position corresponding to position D917, E1006, D1255, R1218, D1226, or two or more thereof.

212. The RNA binding protein of claim 184, wherein the CRISPR protein is a catalytically inactive Cas13a protein.

213. The RNA binding protein of claim 212, wherein the catalytically inactive Cas13a protein is from Leptotrichia buccalis or Leptotrichia wadei.

214. The RNA binding protein of claim 213, wherein the catalytically inactive Cas13a protein from Leptotrichia buccalis comprises the unnatural amino acid sidechain at a position corresponding to position K47, R472, H473, H477, S522, D590, Q659, V810, K855, Q904, R1046, H1053, R1135, or two or more thereof, and the catalytically inactive Cas13a protein from Leptotrichia wadei comprises the unnatural amino acid sidechain at a position corresponding to position K47, R474, H475, H479, S524, D586, Q653, V808, K853, Q902, R1046, H1051, R1133, or two or more thereof.

215. The RNA binding protein of claim 184, wherein the CRISPR protein is a catalytically inactive Cas13d protein.

216. The RNA binding protein of claim 215, wherein the catalytically inactive Cas13d protein is from Eubacterium siraeum.

217. The RNA binding protein of claim 216, wherein the catalytically inactive Cas13d protein from Eubacterium siraeum comprises the unnatural amino acid sidechain at a position corresponding to position R84, N86, R386, N405, T524, N641, R679, Y680, or two or more thereof.

218. The RNA binding protein of claim 184, wherein the RNA binding protein is the RNA chaperone.

219. The RNA binding protein of claim 218, wherein the RNA chaperone is a Hfq protein.

220. The RNA binding protein of claim 219, wherein the Hfq protein comprises the unnatural amino acid sidechain at a position corresponding to position 25, position 30, or position 49.

221. A nucleic acid encoding the CRISPR protein of claim 184.

222. A vector comprising the nucleic acid sequence of claim 221.

223. A biomolecule conjugate of Formula (III): 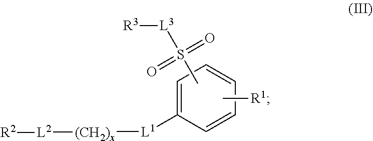
wherein: R² is a CRISPR protein moiety or a RNA chaperone moiety; R³ is a RNA moiety; L¹ is a bond, substituted or unsubstituted alkylene, or substituted or unsubstituted heteroalkylene; x is an integer from 1 to 8; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; v1 is 1 or 2; L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

224. The biomolecule conjugate of claim 223, wherein L⁴ is a bond.

225. The biomolecule conjugate of claim 223, wherein L⁴ is —O—.

226. The biomolecule conjugate of claim 223, wherein x is an integer from 1 to 4.

227. The biomolecule conjugate of claim 223, wherein x is 1.

228. The biomolecule conjugate of claim 223, wherein L¹ is a bond.

229. The biomolecule conjugate of claim 223, wherein L¹ is substituted or unsubstituted 2 to 6 membered heteroalkylene.

230. The biomolecule conjugate of claim 223, wherein L¹ is —NH—C(O)—(CH₂)_{y}— or —NH—C(O)—O—(CH₂)_{y}—, and y is an integer from 0 to 2.

231. The biomolecule conjugate of claim 223, wherein R¹ is substituted or unsubstituted heteroalkyl.

232. The biomolecule conjugate of claim 223, wherein R¹ is unsubstituted 2 to 8 membered heteroalkyl.

233. The biomolecule conjugate of claim 223, wherein R¹ is —O—(CH₂)ₘCH₃, and m is an integer from 0 to 4.

234. The biomolecule conjugate of claim 223, wherein R¹ is ortho to —S(═O)₂F.

235. The biomolecule conjugate of claim 223, wherein R¹ is hydrogen.

236. The biomolecule conjugate of claim 223, wherein: L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-substituted or unsubstituted heteroarylene; L¹² is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N3, unsubstituted alkyl, unsubstituted heteroalkyl, unsubstituted cycloalkyl, unsubstituted heterocycloalkyl, unsubstituted aryl, or unsubstituted heteroaryl; L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹²-substituted or unsubstituted heteroarylene; and L¹³ is halogen, —CF₃, —CBr₃, —CCl₃, —Cl₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCl₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl.

237. The biomolecule conjugate of claim 223, wherein the biomolecule conjugate of Formula (III) is a biomolecule conjugate of Formula (IIIC):

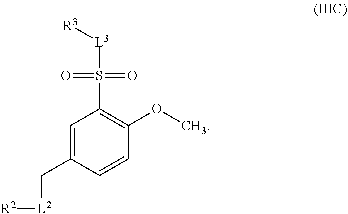

238. The biomolecule conjugate of claim 223, wherein the biomolecule conjugate of Formula (III) is a biomolecule conjugate of Formula (IIIE):

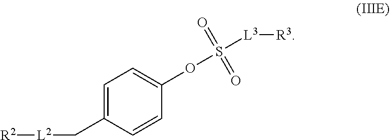

239. The biomolecule conjugate of claim 223, wherein L² is a bond.

240. The biomolecule conjugate of claim 223, wherein L³ is a bond.

241. The biomolecule conjugate of claim 223, wherein the RNA binding protein is the CRISPR protein.

242. The biomolecule conjugate of claim 241, wherein the CRISPR protein comprises the unnatural amino acid sidechain at a position corresponding to position 133.

243. The biomolecule conjugate of claim 241, wherein the CRISPR protein comprises the unnatural amino acid sidechain at a position corresponding to position 380.

244. The biomolecule conjugate of claim 241, wherein the CRISPR protein is a catalytically inactive Cas13b protein.

245. The biomolecule conjugate of claim 244, wherein the catalytically inactive Cas13b protein is from Prevotella sp. P5-125, Bergeyella zoohelcum, or Prevotella buccae.

246. The biomolecule conjugate of claim 244, wherein the catalytically inactive Cas13b protein comprises the unnatural amino acid sidechain at a position corresponding to position 133 or position 380.

247. The biomolecule conjugate of claim 245, wherein the catalytically inactive Cas13b protein from Prevotella sp. P5-125 comprises the unnatural amino acid sidechain at a position corresponding to position R128, H133, R380, R1053, H1058, or two or more thereof; the catalytically inactive Cas13b protein from Bergeyella zoohelcum comprises the unnatural amino acid sidechain at a position corresponding to position R116, H121, R459, R1177, H1182, or two or more thereof; and the catalytically inactive Cas13b protein from Prevotella buccae comprises the unnatural amino acid sidechain at a position corresponding to position R156, H161, K393, R402, R1068, H1073, or two or more thereof.

248. The biomolecule conjugate of claim 241, wherein the CRISPR protein is a catalytically inactive Cas9 protein.

249. The biomolecule conjugate of claim 248, wherein the catalytically inactive Cas9 protein is from Streptococcus pyogenes, Staphylococcus aureus, or Actinomyces naeslundii.

250. The biomolecule conjugate of claim 249, wherein the catalytically inactive Cas9 protein from Streptococcus pyogenes comprises the unnatural amino acid sidechain at a position corresponding to position D10, E762, H983, D986, H840, N863, D839, or two or more thereof; the catalytically inactive Cas9 protein from Staphylococcus aureus comprises the unnatural amino acid sidechain at a position corresponding to position D10, E477, H701, D704, H557, N580, D556, or two or more thereof; and the catalytically inactive Cas9 protein from Actinomyces naeslundii comprises the unnatural amino acid sidechain at a position corresponding to position D17, E505, H736, D739, H582, N606, D581, or two or more thereof.

251. The biomolecule conjugate of claim 241, wherein the CRISPR protein is a catalytically inactive Cas12a protein.

252. The biomolecule conjugate of claim 251, wherein the catalytically inactive Cas12a protein is from Acidaminococcus sp. BV3L6, Lachnospiraceae bacterium ND2006, or Francisella novicida U112.

253. The biomolecule conjugate of claim 252, wherein the catalytically inactive Cas12a protein from Acidaminococcus sp. BV3L6 comprises the unnatural amino acid sidechain at a position corresponding to position D908, E993, D1263, R1226, D1235, or two or more thereof; the catalytically inactive Cas12a protein from Lachnospiraceae bacterium ND2006 comprises the unnatural amino acid sidechain at a position corresponding to position D833, E926, D1181, R1139, D1149, or two or more thereof; and the catalytically inactive Cas12a protein from Francisella novicida U112 comprises the unnatural amino acid sidechain at a position corresponding to position D917, E1006, D1255, R1218, D1226, or two or more thereof.

254. The biomolecule conjugate of claim 241, wherein the CRISPR protein is a catalytically inactive Cas13a protein.

255. The biomolecule conjugate of claim 254, wherein the catalytically inactive Cas13a protein is from Leptotrichia buccalis or Leptotrichia wadei.

256. The biomolecule conjugate of claim 255, wherein the catalytically inactive Cas13a protein from Leptotrichia buccalis comprises the unnatural amino acid sidechain at a position corresponding to position K47, R472, H473, H477, S522, D590, Q659, V810, K855, Q904, R1046, H1053, R1135, or two or more thereof; and the catalytically inactive Cas13a protein from Leptotrichia wadei comprises the unnatural amino acid sidechain at a position corresponding to position K47, R474, H475, H479, S524, D586, Q653, V808, K853, Q902, R1046, H1051, R1133, or two or more thereof.

257. The biomolecule conjugate of claim 241, wherein the CRISPR protein is a catalytically inactive Cas13d protein.

258. The biomolecule conjugate of claim 257, wherein the catalytically inactive Cas13d protein is from Eubacterium siraeum.

259. The biomolecule conjugate of claim 258, wherein the catalytically inactive Cas13d protein from Eubacterium siraeum comprises the unnatural amino acid sidechain at a position corresponding to position R84, N86, R386, N405, T524, N641, R679, Y680, or two or more thereof.

260. The biomolecule conjugate of claim 223, wherein the RNA binding protein is the RNA chaperone.

261. The biomolecule conjugate of claim 260, wherein the RNA chaperone is a Hfq protein.

262. The biomolecule conjugate of claim 261, wherein L² is bonded to the Hfq protein at a position corresponding to position 25, position 30, or position 49.

263. A method of forming the biomolecule conjugate of claim 223, the method comprising contacting the RNA-binding protein of claim 184, RNA, and a guide RNA (crRNA), thereby forming the biomolecule conjugate.

264. A cell comprising: (i) the RNA-binding protein of any one of claims 184 to 220; (ii) the nucleic acid of claim 221; (iii) the vector of claim 222; or (iv) the biomolecule conjugate of any one of claims 223 to 262.


SEQUENCE LISTING (544 sequences)

SEQ ID NO: 1 - protein, 740 aa, Artificial Sequence
  Synthetic Polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 2 - protein, 740 aa, Artificial Sequence
  Synthetic Polypeptide
  misc_feature (34)..(34): Xaa is FSY
MSSSSWLLLSLVAVTAAQSTIEEQAKTFLDKFNXEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 740 aa, Artificial Sequence
  Synthetic Polypeptide
  misc_feature (37)..(37): Xaa is FSY
MSSSSWLLLSLVAVTAAQSTIEEQAKTFLDKFNHEAXDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 740 aa, Artificial Sequence
  Synthetic Polypeptide
  misc_feature (42)..(42): Xaa is FSY
MSSSSWLLLSLVAVTAAQSTIEEQAKTFLDKFNHEAEDLFYXSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 5 - protein, 223 aa, Artificial Sequence
  Synthetic Polypeptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 6 - DNA, 48 bp, Artificial Sequence
  Synthetic Polynucleotide
ctagcgtttaaacttaagcttgccaccatgtcaagctcttcctggctc

SEQ ID NO: 7 - DNA, 57 bp, Artificial Sequence
  Synthetic Polynucleotide
cacactggactagtggatccttagtgatggtgatgatgatgggaaacagggggctgg

SEQ ID NO: 8 - DNA, 30 bp, Artificial Sequence
  Synthetic Polynucleotide
ccaagacatttttgtagaagtttaaccacg

SEQ ID NO: 9 - DNA, 27 bp, Artificial Sequence
  Synthetic Polynucleotide
ctacaaaaatgtcttggcctgttcctc

SEQ ID NO: 10 - DNA, 36 bp, Artificial Sequence
  Synthetic Polynucleotide
gtttaaccacgaagccgaatagctgttctatcaaag

SEQ ID NO: 11 - DNA, 25 bp, Artificial Sequence
  Synthetic Polynucleotide
ctattcggcttcgtggttaaacttg

SEQ ID NO: 12 - DNA, 39 bp, Artificial Sequence
  Synthetic Polynucleotide
caagtttaaccacgaagcctaggacctgttctatcaaag

SEQ ID NO: 13 - DNA, 24 bp, Artificial Sequence
  Synthetic Polynucleotide
ctaggcttcgtggttaaacttgtc

SEQ ID NO: 14 - DNA, 38 bp, Artificial Sequence
  Synthetic Polynucleotide
catttttggacaagtttaactaggaagccgaagacctg

SEQ ID NO: 15 - DNA, 29 bp, Artificial Sequence
  Synthetic Polynucleotide
ggcttcctagttaaacttgtccaaaaatg

SEQ ID NO: 16 - DNA, 33 bp, Artificial Sequence
  Synthetic Polynucleotide
cgaagacctgttctattagagttcacttgcttc

SEQ ID NO: 17 - DNA, 29 bp, Artificial Sequence
  Synthetic Polynucleotide
ctctaatagaacaggtcttcggcttcgtg

SEQ ID NO: 18 - DNA, 33 bp, Artificial Sequence
  Synthetic Polynucleotide
cacacttgcccaaatgtagccactacaagaaat

SEQ ID NO: 19 - DNA, 28 bp, Artificial Sequence
  Synthetic Polynucleotide
gtggctacatttgggcaagtgtggactg

SEQ ID NO: 20 - DNA, 2238 bp, Artificial Sequence
  Synthetic Polynucleotide
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcccatcatcatcaccatcac

SEQ ID NO: 21 - DNA, 2236 bp, Artificial Sequence
  Synthetic Polynucleotide
  misc_feature (88)..(88): n is D30TAG
atgtcaagctcttcctggctccttctcagccttgttgctgtaactgctgctcagtccaccattgaggaacaggccaagacatttttgnaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcccatcatcatcaccatcac

SEQ ID NO: 22 - protein, 740 aa, Artificial Sequence
  Synthetic Polypeptide
  misc_feature (30)..(30): Xaa is FSY
MSSSSWLLLSLVAVTAAQSTIEEQAKTFLXKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 23 - DNA, 2236 bp, Artificial Sequence
  Synthetic Polynucleotide
  misc_feature (100)..(100): n is H34TAG
atgtcaagctcttcctggctccttctcagccttgttgctgtaactgctgctcagtccaccattgaggaacaggccaagacatttttggacaagtttaacngaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcccatcatcatcaccatcac

SEQ ID NO: 24 - DNA, 2236 bp, Artificial Sequence
  Synthetic Polynucleotide
  misc_feature (109)..(109): n is E37TAG
atgtcaagctcttcctggctccttctcagccttgttgctgtaactgctgctcagtccaccattgaggaacaggccaagacatttttggacaagtttaaccacgaagccngacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcccatcatcatcaccatcac

SEQ ID NO: 25 - DNA, 2236 bp, Artificial Sequence
  Synthetic Polynucleotide
  misc_feature (112)..(112): n is D38TAG
atgtcaagctcttcctggctccttctcagccttgttgctgtaactgctgctcagtccaccattgaggaacaggccaagacatttttggacaagtttaaccacgaagccgaanctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcccatcatcatcaccatcac

SEQ ID NO: 26 - protein, 740 aa, Artificial Sequence
  Synthetic Polypeptide
  misc_feature (38)..(38): Xaa is FSY
MSSSSWLLLSLVAVTAAQSTIEEQAKTFLDKFNHEAEXLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 27 - DNA, 2236 bp, Artificial Sequence
  Synthetic Polynucleotide
  misc_feature (124)..(124): n is Q42TAG
atgtcaagctcttcctggctccttctcagccttgttgctgtaactgctgctcagtccaccattgaggaacaggccaagacatttttggacaagtttaaccacgaagccgaagacctgttctatnagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcccatcatcatcaccatcac

SEQ ID NO: 28 - DNA, 2236 bp, Artificial Sequence
  Synthetic Polynucleotide
  misc_feature (247)..(247): n is Y83TAG
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgnccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttcccatcatcatcaccatcac

SEQ ID NO: 29 - protein, 740 aa, Artificial Sequence
  Synthetic Polypeptide
  misc_feature (83)..(83): Xaa is FSY
MSSSSWLLLSLVAVTAAQSTIEEQAKTFLDKFNHEAEDLFYQSSLASWNYNTNITEENVQNMNNAGDKWSAFLKEQSTLAQMXPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 30 - protein, 134 aa, Artificial Sequence
  Synthetic Polypeptide
QVQLVESGGGLVQAGGSLRLSCAASGFPVYSWNMWWYRQAPGKEREWVAAIESHGDSTRYADSVKGRFTISRDNAKNTVYLQMNSLKPEDTAVYYCYVWVGHTYYGQGTQVTVSAGRAGEQKLISEEDLNSAVD

SEQ ID NO: 31 - protein, 10 aa, Artificial Sequence
  Synthetic Polypeptide
GFPVYSWNMW

SEQ ID NO: 32 - protein, 10 aa, Artificial Sequence
  Synthetic Polypeptide
AIESHGDSTR

SEQ ID NO: 33 - protein, 7 aa, Artificial Sequence
  Synthetic Polypeptide
VWVGHTY

SEQ ID NO: 34 - protein, 140 aa, Artificial Sequence
  Synthetic Polypeptide
QVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDDGQLAYHYDYWGQGTQVTVSAGRAGEQKLISEEDLNSAVD

SEQ ID NO: 35 - protein, 10 aa, Artificial Sequence
  Synthetic Polypeptide
GFPVEVWRME

SEQ ID NO: 36 - protein, 10 aa, Artificial Sequence
  Synthetic Polypeptide
AIESYGHGTR

SEQ ID NO: 37 - protein, 13 aa, Artificial Sequence
  Synthetic Polypeptide
VYDDGQLAYHYDY

SEQ ID NO: 38 - protein, 128 aa, Artificial Sequence
  Synthetic Polypeptide
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDYWGQGTQVTVSSK

SEQ ID NO: 39 - protein, 10 aa, Artificial Sequence
  Synthetic Polypeptide
GRTFSTAAMG

SEQ ID NO: 40 - protein, 10 aa, Artificial Sequence
  Synthetic Polypeptide
AIRWSGGSAY

SEQ ID NO: 41 - protein, 19 aa, Artificial Sequence
  Synthetic Polypeptide
RTENVRSLLSDYATWPYDY

SEQ ID NO: 42 - DNA, 468 bp, Artificial Sequence
  Synthetic Polynucleotide
atgaaatatctgctgccaaccgcggccgcgggtctgctgctgctggcggcccaaccagcgatggcgcaagtgcagctggttgagagcggcggtggtctgatgcaagcgggtggtagtctgcgtctgagttgcgcggttagcggtcgcacctttagcaccgccgcgatgggttggtttcgccaagcgccgggcaaagaacgcgaatttgttgcggccatccgttggagcggtggtagtgcgtactacgcggatagcgttaaaggccgcttcaccatcagccgcgataaggcgaagaacaccgtgtatctgcagatgaacagtctgaagtacgaggacaccgccgtgtactattgcgcgcgcaccgaaaacgttcgtagtctgctgagcgattacgccacgtggccgtacgattactggggtcaaggcacccaagttaccgtgagcagcaaacaccaccatcaccatcat

SEQ ID NO: 43 - DNA, 504 bp, Artificial Sequence
  Synthetic Polynucleotide
atgaaatatctgctgccaacggccgcggcgggtctgctgctgctggcggcgcaaccagcgatggcccaagttcagctggttgaaagcggtggcggtctggttcaagccggtggtagtctgcgtctgagctgcgccgccagtggctttccggtggaagtttggcgcatggaatggtaccgccaagccccgggcaaagaacgcgaaggcgttgccgccatcgaaagctacggtcatggcacccgctacgccgatagcgttaaaggccgcttcaccatcagccgcgacaacgcgaagaacaccgtgtatctgcagatgaacagtctgaaaccggaggatacggccgtgtactactgcaacgtgtacgatgatggccagctggcgtaccattacgattactggggccaaggcacccaagttaccgttagtgcgggtcgcgcgggcgaacagaagctgatcagcgaagaggatctgaatagcgccgtggatcaccatcatcatcaccat

SEQ ID NO: 44 - DNA, 486 bp, Artificial Sequence
  Synthetic Polynucleotide
atgaaatatctgctgccaaccgccgcggcgggtctgctgctgctggcggcgcaaccagcgatggcccaagttcagctggttgaaagcggtggtggtctggttcaagccggtggtagtctgcgtctgagctgcgcggcgagtggctttccagtgtacagctggaacatgtggtggtaccgccaagccccgggtaaagaacgcgaatgggttgcggcgatcgaaagccacggcgatagcacccgctacgcggatagcgttaaaggccgcttcaccatcagccgcgacaacgccaagaacaccgtgtatctgcagatgaacagtctgaaaccggaagataccgcggtgtactactgctacgtgtgggttggccacacctactacggtcaaggcacccaagttaccgttagcgcgggtcgtgcgggcgaacagaagctgatcagcgaggaagatctgaacagcgccgtggatcaccatcaccatcatcat

SEQ ID NO: 45 - protein, 1133 aa, Artificial Sequence
  Synthetic Polypeptide
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMKGSLQLPPLERLTLGSSYPYDVPDYAYPYDVPDYAYPYDVPDYA

SEQ ID NO: 46 - protein, 1133 aa, Artificial Sequence
  Synthetic Polypeptide
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMKGSLQLPPLERLTLGSSYPYDVPDYAYPYDVPDYAYPYDVPDYA

SEQ ID NO: 47 - protein, 1133 aa, Artificial Sequence
  Synthetic Polypeptide
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSSYPYDVPDYAYPYDVPDYAYPYDVPDYA

SEQ ID NO: 48 - protein, 1133 aa, Artificial Sequence
  Synthetic Polypeptide
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSSYPYDVPDYAYPYDVPDYAYPYDVPDYA

SEQ ID NO: 49 - protein, 452 aa, Artificial Sequence
  Synthetic Polypeptide
  misc_feature (1)..(452): Xaa are mutated residues
DKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNXYNYLRKLDRALPDPIKXFEIGPCYRKESDGKEHLEEFTMLXFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 50 - protein, 126 aa, Artificial Sequence
  Synthetic Polypeptide
  misc_feature (38)..(39): Xaa are amber codon TAG
MAVDNKFNKEMRNAYWEIALLPNLNNQQKRAFIRSLYXXPSQSANLLAEAKKLNDAQAPKVEVDNKFNKEMRNAYWEIALLPNLNNQQKRAFIRSLYDDPSQSANLLAEAKKLNDAQAPKHHHHHH

SEQ ID NO: 51 - protein, 145 aa, Artificial Sequence
  Synthetic Polypeptide
  misc_feature (37)..(37): Xaa is amber codon TAG
MKYLLPTAAAGLLLLAAQPAMAMGQVQLQESGGGSVXAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSSHHHHHH

SEQ ID NO: 52 - protein, 237 aa, Artificial Sequence
  Synthetic Polypeptide
  misc_feature (1)..(237): Xaa are amber codon TAG
MKSLLPTAAAGLLLLAAQPAMASDIQMTQSPSSLSASVGDRVTITCRASQDVNTAVAWYQQKPGKAPKLLIYXASFLYSGVPSRFSGSRSGTDFTLTISSLQPEDFATYYCQQHXTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 276 aa, Artificial Sequence
  Synthetic Polypeptide
MKKNIAFLLASMFVFSIATNAYAEISEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGGLNDIFEAQKIEWHE

SEQ ID NO: 54 - protein, 154 aa, Artificial Sequence
  Synthetic Polypeptide
  misc_feature (116)..(116): Xaa is amber codon TAG
MKYLLPTAAAGLLLLAAQPAMAMGQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 55 - protein, 207 aa, Artificial Sequence
  Synthetic Polypeptide
  misc_feature (53)..(53): Xaa is amber codon TAG
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGLEVLFQGPSHLVKCAEKEKTFCVNGGECFMVKDLSNPSRYLCKCPNEFTGDRCQNYVMASFYKHLGIEGSGSGSDYKDDDDKAAALEHHHHHH

SEQ ID NO: 56 - protein, 454 aa, Artificial Sequence
  Synthetic Polypeptide
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 57 - DNA, 1365 bp, Artificial Sequence
  Synthetic Polynucleotide
atggataaaaagcctttgaacactctgatttctgcgaccggtctgtggatgtcccgcaccggcaccatccacaaaatcaaacaccatgaagttagccgttccaaaatctacattgaaatggcttgcggcgatcacctggttgtcaacaactcccgttcttctcgtaccgctcgcgcactgcgccaccacaaatatcgcaaaacctgcaaacgttgccgtgttagcgatgaggacctgaacaaattcctgaccaaagctaacgaggatcagacctccgtaaaagtgaaggtagtaagcgctccgacccgtactaaaaaggctatgccaaaaagcgtggcccgtgccccgaaacctctggaaaacaccgaggcggctcaggctcaaccatccggttctaaattttctccggcgatcccagtgtccacccaagaatctgtttccgtaccagcaagcgtgtctaccagcattagcagcatttctaccggtgctaccgcttctgcgctggtaaaaggtaacactaacccgattactagcatgtctgcaccggtacaggcaagcgccccagctctgactaaatcccagacggaccgtctggaggtgctgctgaacccaaaggatgaaatctctctgaacagcggcaagcctttccgtgagctggaaagcgagctgctgtctcgtcgtaaaaaggatctgcaacagatctacgctgaggaacgcgagaactatctgggtaagctggagcgcgaaattactcgcttcttcgtggatcgcggtttcctggagatcaaatctccgattctgattccgctggaatacattgaacgtatgggcatcgataatgataccgaactgtctaaacagatcttccgtgtggataaaaacttctgtctgcgtccgatgctgattccgaacttgtacaactatttacgtaaactggaccgtgccctgccggacccgatcaaaatattcgagatcggtccttgctaccgtaaagagtccgacggtaaagagcacctggaagaattcaccatgctgacattcattcagatgggtagcggttgcacgcgtgaaaacctggaatccattatcaccgacttcctgaatcacctgggtatcgatttcaaaattgttggtgacagctgtatggtgttaggcgatacgctggatgttatgcacggcgatctggagctgtcttccgcagttgtgggcccaatcccgctggatcgtgagtggggtatcgacaaacctaaaatcggtgcgggttttggtctggagcgtctgctgaaagtaaaacacgacttcaagaacatcaaacgtgctgcacgttccgagtcctattacaatggtatttctactaacctgtaa

SEQ ID NO: 58 - protein, 420 aa, Artificial Sequence
  Synthetic Polypeptide
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLIPNLYNYLRKLDRALPDPIKIFEIGPCYRKESDGKEHLEEFTMLTFIQMGSGCTRENLESIITDFLNHLGIDFKIVGDSCMVLGDTLDVMHGDLELSSAVVGPIPLDREWGIDKPKIGA

SEQ ID NO: 59 - DNA, 69 bp, Artificial Sequence
  Synthetic Polynucleotide
ggaaacctgatcatgtagatcgaatggactctaaatccgttcagccgggttagattcccggggtttccg

SEQ ID NO: 60 - protein, 119 aa, Artificial Sequence
  Synthetic Polypeptide
QVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYGDYWGQGTQVTVSS

SEQ ID NO: 61 - protein, 10 aa, Artificial Sequence
  Synthetic Polypeptide
GYIFGRNAMG

SEQ ID NO: 62 - protein, 10 aa, Artificial Sequence
  Synthetic Polypeptide
GITRRGSITY

SEQ ID NO: 63 - protein, 10 aa, Artificial Sequence
  Synthetic Polypeptide
DPASPAYGDY

SEQ ID NO: 64 - protein, 10 aa, Artificial Sequence
  Synthetic Polypeptide
  misc_feature (10)..(10): Xaa is FFY
DPASPAYGDX

SEQ ID NO: 65 - protein, 119 aa, Artificial Sequence
  Synthetic Polypeptide
  misc_feature (108)..(108): Xaa is FFY
QVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYGDXWGQGTQVTVSS

SEQ ID NO: 66 - protein, 115 aa, Artificial Sequence
  Synthetic Polypeptide
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 67 - protein, 8 aa, Artificial Sequence
  Synthetic Polypeptide
GYIFNSCG

SEQ ID NO: 68 - protein, 7 aa, Artificial Sequence
  Synthetic Polypeptide
RISGDGD

SEQ ID NO: 69 - protein, 9 aa, Artificial Sequence
  Synthetic Polypeptide
AVCYNLETY

SEQ ID NO: 70 - protein, 7 aa, Artificial Sequence
  Synthetic Polypeptide
  misc_feature (5)..(5): Xaa is FSY
RISGXGD

SEQ ID NO: 71 - protein, 9 aa, Artificial Sequence
  Synthetic Polypeptide
  misc_feature (7)..(7): Xaa is FSY
AVCYNLXTY

SEQ ID NO: 72 - protein, 115 aa, Artificial Sequence
  Synthetic Polypeptide
  misc_feature (54)..(54): Xaa is FSY
QVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSS

SEQ ID NO: 73 - protein, 115 aa, Artificial Sequence
  Synthetic Polypeptide
  misc_feature (102)..(102): Xaa is FSY
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLXTYWGQGTQVTVSS

SEQ ID NO: 74 - protein, 122 aa, Artificial Sequence
  Synthetic Polypeptide
EVQLVESGGELVQAGGSLRLSCAASGLTFSSYNMGWFRRAPGKEREFVASITWSGRDTFYADSVKGRFTISRDNAKNTVYLQMSSLKPEDTAVYYCAANPWPVAAPRSGTYWGQGTQVTVSS

SEQ ID NO: 75 - protein, 8 aa, Artificial Sequence
  Synthetic Polypeptide
GLTFSSYN

SEQ ID NO: 76 - protein, 8 aa, Artificial Sequence
  Synthetic Polypeptide
ITWSGRDT

SEQ ID NO: 77 - protein, 15 aa, Artificial Sequence
  Synthetic Polypeptide
AANPWPVAAPRSGTY

SEQ ID NO: 78 - protein, 8 aa, Artificial Sequence
  Synthetic Polypeptide
  misc_feature (6)..(6): Xaa is FSY
GLTFSXYN

SEQ ID NO: 79 - protein, 122 aa, Artificial Sequence
  Synthetic Polypeptide
  misc_feature (31)..(31): Xaa is FSY
EVQLVESGGELVQAGGSLRLSCAASGLTFSXYNMGWFRRAPGKEREFVASITWSGRDTFYADSVKGRFTISRDNAKNTVYLQMSSLKPEDTAVYYCAANPWPVAAPRSGTYWGQGTQVTVSS

SEQ ID NO: 80 - protein, 127 aa, Artificial Sequence
  Synthetic Polypeptide
QVQLQESGGGSVQTGGSLRLSCAASGYTASFSWIGYFRQAPGKEREGVAVINVGVGSTYYADSVKGRFTISRDNTENTISLEMNSLKPEDTGLYYCAGSLRWSRPPNPISEDAYNYWGQGTQVTVSS

SEQ ID NO: 81 - protein, 9 aa, Artificial Sequence
  Synthetic Polypeptide
GYTASFSWI

SEQ ID NO: 82 - protein, 9 aa, Artificial Sequence
  Synthetic Polypeptide
VINVGVGST

SEQ ID NO: 83 - protein, 20 aa, Artificial Sequence
  Synthetic Polypeptide
AGSLRWSRPPNPISEDAYNY

SEQ ID NO: 84 - protein, 9 aa, Artificial Sequence
  Synthetic Polypeptide
  misc_feature (5)..(5): Xaa is FSY
VINVXVGST

SEQ ID NO: 85 - protein, 9 aa, Artificial Sequence
  Synthetic Polypeptide
  misc_feature (8)..(8): Xaa is FSY
VINVGVGXT

SEQ ID NO: 86 - protein, 9 aa, Artificial Sequence
  Synthetic Polypeptide
  misc_feature (7)..(7): Xaa is mFSY
GYTASFXWI

SEQ ID NO: 87 - protein, 9 aa, Artificial Sequence
  Synthetic Polypeptide
  misc_feature (5)..(5): Xaa is mFSY
VINVXVGST

SEQ ID NO: 88 - protein, 127 aa, Artificial Sequence
  Synthetic Polypeptide
  misc_feature (54)..(54): Xaa is FSY
QVQLQESGGGSVQTGGSLRLSCAASGYTASFSWIGYFRQAPGKEREGVAVINVXVGSTYYADSVKGRFTISRDNTENTISLEMNSLKPEDTGLYYCAGSLRWSRPPNPISEDAYNYWGQGTQVTVSS

SEQ ID NO: 89 - protein, 127 aa, Artificial Sequence
  Synthetic Polypeptide
  misc_feature (57)..(57): Xaa is FSY
QVQLQESGGGSVQTGGSLRLSCAASGYTASFSWIGYFRQAPGKEREGVAVINVGVGXTYYADSVKGRFTISRDNTENTISLEMNSLKPEDTGLYYCAGSLRWSRPPNPISEDAYNYWGQGTQVTVSS

SEQ ID NO: 90 - protein, 127 aa, Artificial Sequence
  Synthetic Polypeptide
  misc_feature (32)..(32): Xaa is mFSY
QVQLQESGGGSVQTGGSLRLSCAASGYTASFXWIGYFRQAPGKEREGVAVINVGVGSTYYADSVKGRFTISRDNTENTISLEMNSLKPEDTGLYYCAGSLRWSRPPNPISEDAYNYWGQGTQVTVSS

SEQ ID NO: 91 - protein, 127 aa, Artificial Sequence; identical to SEQ ID NO: 88
  Synthetic Polypeptide
  misc_feature (54)..(54): Xaa is mFSY

SEQ ID NO: 92 - protein, 135 aa, Artificial Sequence
  Synthetic Polypeptide
EVQLVESGGGLVQPGGSLRLSCAASGSIGGLNAMAWYRQAPGKERELVAGIFGVGSTRYADSVKGRFTISRDNSKNTVYLQMNSLRSEDTAVYYCAMSSVTRGSSDYWGQGTLVTVSSAAAEQKLISEEDLNGAA

SEQ ID NO: 93 - protein, 5 aa, Artificial Sequence
  Synthetic Polypeptide
LNAMA

SEQ ID NO: 94 - protein, 16 aa, Artificial Sequence
  Synthetic Polypeptide
GIFGVGSTRYADSVKG

SEQ ID NO: 95 - protein, 10 aa, Artificial Sequence
  Synthetic Polypeptide
SSVTRGSSDY

SEQ ID NO: 96 - protein, 16 aa, Artificial Sequence
  Synthetic Polypeptide
  misc_feature (10)..(10): Xaa is FSY
GIFGVGSTRXADSVKG

SEQ ID NO: 97 - protein, 16 aa, Artificial Sequence
  Synthetic Polypeptide
  misc_feature (11)..(11): Xaa is FSY
GIFGVGSTRYXDSVKG

SEQ ID NO: 98 - protein, 16 aa, Artificial Sequence
  Synthetic Polypeptide
  misc_feature (12)..(12): Xaa is FSY
GIFGVGSTRYAXSVKG

SEQ ID NO: 99 - protein, 16 aa, Artificial Sequence
  Synthetic Polypeptide
  misc_feature (13)..(13): Xaa is FSY
GIFGVGSTRYADXVKG

SEQ ID NO: 100 - protein, 16 aa, Artificial Sequence
  Synthetic Polypeptide
  misc_feature (14)..(14): Xaa is FSY
GIFGVGSTRYADSXKG

SEQ ID NO: 101 - protein, 16 aa, Artificial Sequence
  Synthetic Polypeptide
  misc_feature (15)..(15): Xaa is FSY
GIFGVGSTRYADSVXG

SEQ ID NO: 102 - protein, 16 aa, Artificial Sequence
  Synthetic Polypeptide
  misc_feature (16)..(16): Xaa is FSY
GIFGVGSTRYADSVKX

SEQ ID NO: 103 - protein, 10 aa, Artificial Sequence
  Synthetic Polypeptide
  misc_feature (5)..(5): Xaa is FSY
SSVTXGSSDY

SEQ ID NO: 104 - protein, 10 aa, Artificial Sequence
  Synthetic Polypeptide
  misc_feature (6)..(6): Xaa is FSY
SSVTRXSSDY

SEQ ID NO: 105 - protein, 16 aa, Artificial Sequence
  Synthetic Polypeptide
  misc_feature (8)..(8): Xaa is mFSY
GIFGVGSXRYADSVKG

SEQ ID NO: 106 - protein, 16 aa, Artificial Sequence
  Synthetic Polypeptide
  misc_feature (9)..(9): Xaa is mFSY
GIFGVGSTXYADSVKG

SEQ ID NO: 107 - protein, 16 aa, Artificial Sequence
  Synthetic Polypeptide
  misc_feature (10)..(10): Xaa is mFSY
GIFGVGSTRXADSVKG

SEQ ID NO: 108 - protein, 16 aa, Artificial Sequence
  Synthetic Polypeptide
  misc_feature (11)..(11): Xaa is mFSY
GIFGVGSTRYXDSVKG

SEQ ID NO: 109 - protein, 16 aa, Artificial Sequence
  Synthetic Polypeptide
  misc_feature (12)..(12): Xaa is mFSY
GIFGVGSTRYAXSVKG

SEQ ID NO: 110 - protein, 16 aa, Artificial Sequence
  Synthetic Polypeptide
  misc_feature (13)..(13): Xaa is mFSY
GIFGVGSTRYADXVKG

SEQ ID NO: 111 - protein, 16 aa, Artificial Sequence
  Synthetic Polypeptide
  misc_feature (14)..(14): Xaa is mFSY
GIFGVGSTRYADSXKG

SEQ ID NO: 112 - protein, 16 aa, Artificial Sequence
  Synthetic Polypeptide
  misc_feature (15)..(15): Xaa is mFSY
GIFGVGSTRYADSVXG

SEQ ID NO: 113 - protein, 16 aa, Artificial Sequence
  Synthetic Polypeptide
  misc_feature (16)..(16): Xaa is mFSY
GIFGVGSTRYADSVKX

SEQ ID NO: 114 - protein, 10 aa, Artificial Sequence
  Synthetic Polypeptide
  misc_feature (5)..(5): Xaa is mFSY
SSVTXGSSDY

SEQ ID NO: 115 - protein, 10 aa, Artificial Sequence
  Synthetic Polypeptide
  misc_feature (6)..(6): Xaa is mFSY
SSVTRXSSDY

SEQ ID NO: 116 - protein, 135 aa, Artificial Sequence
  Synthetic Polypeptide
  misc_feature (59)..(59): Xaa is FSY
EVQLVESGGGLVQPGGSLRLSCAASGSIGGLNAMAWYRQAPGKERELVAGIFGVGSTRXADSVKGRFTISRDNSKNTVYLQMNSLRSEDTAVYYCAMSSVTRGSSDYWGQGTLVTVSSAAAEQKLISEEDLNGAA

SEQ ID NO: 117 - protein, 135 aa, Artificial Sequence
  Synthetic Polypeptide
  misc_feature (60)..(60): Xaa is FSY
EVQLVESGGGLVQPGGSLRLSCAASGSIGGLNAMAWYRQAPGKERELVAGIFGVGSTRYXDSVKGRFTISRDNSKNTVYLQMNSLRSEDTAVYYCAMSSVTRGSSDYWGQGTLVTVSSAAAEQKLISEEDLNGAA

SEQ ID NO: 118 - protein, 135 aa, Artificial Sequence
  Synthetic Polypeptide
  misc_feature (61)..(61): Xaa is FSY
EVQLVESGGGLVQPGGSLRLSCAASGSIGGLNAMAWYRQAPGKERELVAGIFGVGSTRYAXSVKGRFTISRDNSKNTVYLQMNSLRSEDTAVYYCAMSSVTRGSSDYWGQGTLVTVSSAAAEQKLISEEDLNGAA

SEQ ID NO: 119 - protein, 135 aa, Artificial Sequence
  Synthetic Polypeptide
  misc_feature (62)..(62): Xaa is FSY
EVQLVESGGGLVQPGGSLRLSCAASGSIGGLNAMAWYRQAPGKERELVAGIFGVGSTRYADXVKGRFTISRDNSKNTVYLQMNSLRSEDTAVYYCAMSSVTRGSSDYWGQGTLVTVSSAAAEQKLISEEDLNGAA

SEQ ID NO: 120 - protein, 135 aa, Artificial Sequence
  Synthetic Polypeptide
  misc_feature (103)..(103): Xaa is FSY
EVQLVESGGGLVQPGGSLRLSCAASGSIGGLNAMAWYRQAPGKERELVAGIFGVGSTRYADSVKGRFTISRDNSKNTVYLQMNSLRSEDTAVYYCAMSSVTRXSSDYWGQGTLVTVSSAAAEQKLISEEDLNGAA

SEQ ID NO: 121 - protein, 135 aa, Artificial Sequence
  Synthetic Polypeptide
  misc_feature (57)..(57): Xaa is mFSY
EVQLVESGGGLVQPGGSLRLSCAASGSIGGLNAMAWYRQAPGKERELVAGIFGVGSXRYADSVKGRFTISRDNSKNTVYLQMNSLRSEDTAVYYCAMSSVTRGSSDYWGQGTLVTVSSAAAEQKLISEEDLNGAA

SEQ ID NO: 122 - protein, 135 aa, Artificial Sequence
  Synthetic Polypeptide
  misc_feature (58)..(58): Xaa is mFSY
EVQLVESGGGLVQPGGSLRLSCAASGSIGGLNAMAWYRQAPGKERELVAGIFGVGSTXYADSVKGRFTISRDNSKNTVYLQMNSLRSEDTAVYYCAMSSVTRGSSDYWGQGTLVTVSSAAAEQKLISEEDLNGAA

SEQ ID NO: 123 - protein, 135 aa, Artificial Sequence; identical to SEQ ID NO: 116
  Synthetic Polypeptide
  misc_feature (59)..(59): Xaa is mFSY

SEQ ID NO: 124 - protein, 135 aa, Artificial Sequence
  Synthetic Polypeptide
  misc_feature (64)..(64): Xaa is mFSY
EVQLVESGGGLVQPGGSLRLSCAASGSIGGLNAMAWYRQAPGKERELVAGIFGVGSTRYADSVXGRFTISRDNSKNTVYLQMNSLRSEDTAVYYCAMSSVTRGSSDYWGQGTLVTVSSAAAEQKLISEEDLNGAA

SEQ ID NO: 125 - protein, 135 aa, Artificial Sequence
  Synthetic Polypeptide
  misc_feature (65)..(65): Xaa is mFSY
EVQLVESGGGLVQPGGSLRLSCAASGSIGGLNAMAWYRQAPGKERELVAGIFGVGSTRYADSVKXRFTISRDNSKNTVYLQMNSLRSEDTAVYYCAMSSVTRGSSDYWGQGTLVTVSSAAAEQKLISEEDLNGAA

SEQ ID NO: 126 - protein, 135 aa, Artificial Sequence
  Synthetic Polypeptide
  misc_feature (102)..(102): Xaa is mFSY
EVQLVESGGGLVQPGGSLRLSCAASGSIGGLNAMAWYRQAPGKERELVAGIFGVGSTRYADSVKGRFTISRDNSKNTVYLQMNSLRSEDTAVYYCAMSSVTXGSSDYWGQGTLVTVSSAAAEQKLISEEDLNGAA

SEQ ID NO: 127 - protein, 135 aa, Artificial Sequence; identical to SEQ ID NO: 120
  Synthetic Polypeptide
  misc_feature (103)..(103): Xaa is mFSY

SEQ ID NO: 128 - protein, 24 aa, Artificial Sequence
  Synthetic Polypeptide
MKYLLPTAAAGLLLLAAQPAMAMA

SEQ ID NO: 129 - protein, 122 aa, Artificial Sequence
  Synthetic Polypeptide
EVQLVESGGGLVQPGGSLRLSCAASGTLFKINAMGWYRQAPGKRRELVALITSSDTTDYADSVKGRFTISRDNSWNTVYLQMNSLRPEDTAVYYCHSDHYSLGVPEKRVILYGQGTLVTVSS

SEQ ID NO: 130 - protein, 267 aa, Artificial Sequence
  Synthetic Polypeptide
  misc_feature (192)..(192): Xaa is FSY
EVQLVESGGGLVQPGGSLRLSCAASGTLFKINAMGWYRQAPGKRRELVALITSSDTTDYADSVKGRFTISRDNSWNTVYLQMNSLRPEDTAVYYCHSDHYSLGVPEKRVILYGQGTLVTVSSGGGGSGGGGSEVQLVESGGGLVQPGGSLRLSCAASGSIGGLNAMAWYRQAPGKERELVAGIFGVGSTRYXDSVKGRFTISRDNSKNTVYLQMNSLRSEDTAVYYCRMSSVTRGSSDYWGQGTLVTVSSAAAEQKLISEEDLNGAA

SEQ ID NO: 131 - protein, 269 aa, Artificial Sequence
  Synthetic Polypeptide
  misc_feature (194)..(194): Xaa is FSY
EVQLVESGGGLVQPGGSLRLSCAASGTLFKINAMGWYRQAPGKRRELVALITSSDTTDYADSVKGRFTISRDNSWNTVYLQMNSLRPEDTAVYYCHSDHYSLGVPEKRVILYGQGTLVTVSSGGGSGGGSGGGSEVQLVESGGGLVQPGGSLRLSCAASGSIGGLNAMAWYRQAPGKERELVAGIFGVGSTRYXDSVKGRFTISRDNSKNTVYLQMNSLRSEDTAVYYCRMSSVTRGSSDYWGQGTLVTVSSAAAEQKLISEEDLNGAA

SEQ ID NO: 132 - protein, 272 aa, Artificial Sequence
  Synthetic Polypeptide
  misc_feature (197)..(197): Xaa is FSY
EVQLVESGGGLVQPGGSLRLSCAASGTLFKINAMGWYRQAPGKRRELVALITSSDTTDYADSVKGRFTISRDNSWNTVYLQMNSLRPEDTAVYYCHSDHYSLGVPEKRVILYGQGTLVTVSSGGGGSGGGGSGGGGSEVQLVESGGGLVQPGGSLRLSCAASGSIGGLNAMAWYRQAPGKERELVAGIFGVGSTRYXDSVKGRFTISRDNSKNTVYLQMNSLRSEDTAVYYCRMSSVTRGSSDYWGQGTLVTVSSAAAEQKLISEEDLNGAA

SEQ ID NO: 133 - protein, 277 aa, Artificial Sequence
  Synthetic Polypeptide
  misc_feature (202)..(202): Xaa is FSY
EVQLVESGGGLVQPGGSLRLSCAASGTLFKINAMGWYRQAPGKRRELVALITSSDTTDYADSVKGRFTISRDNSWNTVYLQMNSLRPEDTAVYYCHSDHYSLGVPEKRVILYGQGTLVTVSSGGGGSGGGGSGGGGSGGGGSEVQLVESGGGLVQPGGSLRLSCAASGSIGGLNAMAWYRQAPGKERELVAGIFGVGSTRYXDSVKGRFTISRDNSKNTVYLQMNSLRSEDTAVYYCRMSSVTRGSSDYWGQGTLVTVSSAAAEQKLISEEDLNGAA

SEQ ID NO: 134 - protein, 249 aa, Artificial Sequence
  Synthetic Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGSGGGGGGSQVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 135 - protein, 249 aa, Artificial Sequence
  Synthetic Polypeptide
  misc_feature (188)..(188): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGSGGGGGGSQVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSS

SEQ ID NO: 136 - protein, 252 aa, Artificial Sequence
  Synthetic Polypeptide
  misc_feature (1)..(252): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXFSYEYDYWGQGTQVTVSSGGSGGGGGGSQVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSS

SEQ ID NO: 137 - protein, 56 aa, Artificial Sequence
  Synthetic Polypeptide
AEAKYAKEMRNAYWEIALLPNLTNQQKRAFIRKLYDDPSQSSELLSEAKKLNDSQA

SEQ ID NO: 138 - protein, 58 aa, Artificial Sequence
  Synthetic Polypeptide
VDNKFNKEMRNAYWEIALLPNLNNQQKRAFIRSLYDDPSQSANLLAEAKKLNDAQAPK

SEQ ID NO: 139 - protein, 118 aa, Artificial Sequence
  Synthetic Polypeptide
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 140 - protein, 192 aa, Artificial Sequence
  Synthetic Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGSGGGGGGSAEAKYAKEMRNAYWEIALLPNLTNQQKRAFIRKLYDDPSQSSELLSEAKKLNDSQAPK

SEQ ID NO: 141 - protein, 192 aa, Artificial Sequence
  Synthetic Polypeptide
  misc_feature (109)..(109): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSGGSGGGGGGSAEAKYAKEMRNAYWEIALLPNLTNQQKRAFIRKLYDDPSQSSELLSEAKKLNDSQAPK

SEQ ID NO: 142 - protein, 192 aa, Artificial Sequence
  Synthetic Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGSGGGGGGSVDNKFNKEMRNAYWEIALLPNLNNQQKRAFIRSLYDDPSQSANLLAEAKKLNDAQAPK

SEQ ID NO: 143 - protein, 192 aa, Artificial Sequence
  Synthetic Polypeptide
  misc_feature (109)..(109): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSGGSGGGGGGSVDNKFNKEMRNAYWEIALLPNLNNQQKRAFIRSLYDDPSQSANLLAEAKKLNDAQAPK

SEQ ID NO: 144 - skipped

SEQ ID NO: 145 - protein, 252 aa, Artificial Sequence
  Synthetic Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGSGGGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 146 - protein, 252 aa, Artificial Sequence
  Synthetic Polypeptide
  misc_feature (109)..(109): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSSGGSGGGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 147 - protein, 241 aa, Artificial Sequence
  Synthetic Polypeptide
VDNKFNKEMRNAYWEIALLPNLNNQQKRAFIRSLYDDPSQSANLLAEAKKLNDAQAPKVDNKFNKEMRNAYWEIALLPNLNNQQKRAFIRSLYDDPSQSANLLAEAKKLNDAQAPKGGSGGGGGGSQVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 148 - protein, 246 aa, Artificial Sequence
  Synthetic Polypeptide
  misc_feature (1)..(246): Xaa are FSY
VDNKFNKEMRNAYWEIALLPNLNNQQKRAFIRSLYDXPSQSANLLAEAKKLNDAQAPKGGGSGVDNKFNKEMRNAYWEIALLPNLNNQQKRAFIRSLYDDPSQSANLLAEAKKLNDAQAPKGGSGGGGGGSQVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSS

SEQ ID NO: 149 - protein, 183 aa, Artificial Sequence
  Synthetic Polypeptide
VDNKFNKEMRNAYWEIALLPNLNNQQKRAFIRSLYDDPSQSANLLAEAKKLNDAQAPKGGSGGGGGGSQVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 150 - protein, 183 aa, Artificial Sequence
  Synthetic Polypeptide
  misc_feature (1)..(183): Xaa are FSY
VDNKFNKEMRNAYWEIALLPNLNNQQKRAFIRSLYDXPSQSANLLAEAKKLNDAQAPKGGSGGGGGGSQVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSS

SEQ ID NO: 151 - protein, 246 aa, Artificial Sequence
  Synthetic Polypeptide
  misc_feature (185)..(185): Xaa is FSY
VDNKFNKEMRNAYWEIALLPNLNNQQKRAFIRSLYDDPSQSANLLAEAKKLNDAQAPKGGGSGVDNKFNKEMRNAYWEIALLPNLNNQQKRAFIRSLYDDPSQSANLLAEAKKLNDAQAPKGGSGGGGGGSQVQLQESGGGSVQAGGSLKLTCAASGYIFNSCGMGWYRQSPGRERELVSRISGXGDTWHKESVKGRFTISQDNVKKTLYLQMNSLKPEDTAVYFCAVCYNLETYWGQGTQVTVSS

SEQ ID NO: 152 - protein, 255 aa, Artificial Sequence
  Synthetic Polypeptide
VDNKFNKEMRNAYWEIALLPNLNNQQKRAFIRSLYDDPSQSANLLAEAKKLNDAQAPKGGGSGVDNKFNKEMRNAYWEIALLPNLNNQQKRAFIRSLYDDPSQSANLLAEAKKLNDAQAPKGGSGGGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 153 - protein, 255 aa, Artificial Sequence
  Synthetic Polypeptide
  misc_feature (240)..(240): Xaa is FSY
VDNKFNKEMRNAYWEIALLPNLNNQQKRAFIRSLYDDPSQSANLLAEAKKLNDAQAPKGGGSGVDNKFNKEMRNAYWEIALLPNLNNQQKRAFIRSLYDDPSQSANLLAEAKKLNDAQAPKGGSGGGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSS

SEQ ID NO: 154 - protein, 124 aa, Artificial Sequence
  Synthetic Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 155 - protein, 9 aa, Artificial Sequence
  Synthetic Polypeptide
RTSRSYGMG

SEQ ID NO: 156 - protein, 8 aa, Artificial Sequence
  Synthetic Polypeptide
GISWRGDS

SEQ ID NO: 157 - protein, 15 aa, Artificial Sequence
  Synthetic Polypeptide
AAGSAWYGTLYEYDY

SEQ ID NO: 158 - protein, 15 aa, Artificial Sequence
  Synthetic Polypeptide
  misc_feature (11)..(11): Xaa is FSY
AAGSAWYGTLXEYDY

SEQ ID NO: 159 - protein, 15 aa, Artificial Sequence
  Synthetic Polypeptide
  misc_feature (15)..(15): Xaa is FSY
AAGSAWYGTLYEYDX

SEQ ID NO: 160 - protein, 124 aa, Artificial Sequence
  Synthetic Polypeptide
  misc_feature (109)..(109): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLXEYDYWGQGTQVTVSS

SEQ ID NO: 161 - protein, 124 aa, Artificial Sequence
  Synthetic Polypeptide
  misc_feature (113)..(113): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDXWGQGTQVTVSS

SEQ ID NO: 162 - protein, 214 aa, Artificial Sequence
  Synthetic Polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 11 aa, Artificial Sequence
  Synthetic Polypeptide
RASQDVNTAVA

SEQ ID NO: 164 - protein, 6 aa, Artificial Sequence
  Synthetic Polypeptide
SASFLY

SEQ ID NO: 165 - protein, 8 aa, Artificial Sequence
  Synthetic Polypeptide
QQHYTTPP

SEQ ID NO: 166 - protein, 8 aa, Artificial Sequence
  Synthetic Polypeptide
  misc_feature (4)..(4): Xaa is FSY
QQHXTTPP

SEQ ID NO: 167 - protein, 8 aa, Artificial Sequence
  Synthetic Polypeptide
  misc_feature (4)..(4): Xaa is mFSY
QQHXTTPP

SEQ ID NO: 168 - protein, 214 aa, Artificial Sequence
  Synthetic Polypeptide
  misc_feature (92)..(92): Xaa is FSY
DIQMTQSPSSLSASVGDRVTITCRASQDVNTAVAWYQQKPGKAPKLLIYSASFLYSGVPSRFSGSRSGTDFTLTISSLQPEDFATYYCQQHXTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 214 aa, Artificial Sequence; identical to SEQ ID NO: 168
  Synthetic Polypeptide
  misc_feature (92)..(92): Xaa is mFSY

SEQ ID NO: 170 - protein, 253 aa, Artificial Sequence
  Synthetic Polypeptide
EISEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGGLNDIFEAQKIEWHE

SEQ ID NO: 171 - protein, 10 aa, Artificial Sequence
  Synthetic Polypeptide
GFNIKDTYIH

SEQ ID NO: 172 - protein, 17 aa, Artificial Sequence
  Synthetic Polypeptide
RIYPTNGYTRYADSVKG

SEQ ID NO: 173 - protein, 11 aa, Artificial Sequence
  Synthetic Polypeptide
WGGDGFYAMDY

SEQ ID NO: 174 - protein, 200 aa, Artificial Sequence
  Synthetic Polypeptide
  misc_feature (172)..(172): Xaa is mFSY
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGLEVLFQGPSHLVKCAEKEKTFCVNGGECFMVKDLSNPSRYLCKCPNEFTGDRCQNYVMXSFYKHLGIEGSGSGSDYKDDDDKAAALE

SEQ ID NO: 175 - protein, 108 aa, Artificial Sequence
  Synthetic Polypeptide
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 176 - protein, 60 aa, Artificial Sequence
  Synthetic Polypeptide
  misc_feature (51)..(51): Xaa is mFSY
SHLVKCAEKEKTFCVNGGECFMVKDLSNPSRYLCKCPNEFTGDRCQNYVMXSFYKHLGIE

SEQ ID NO: 177 - protein, 146 aa, Artificial Sequence
  Synthetic Polypeptide
MAQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGGGGSGGGGSLPETGG

SEQ ID NO: 178 - protein, 128 aa, Artificial Sequence
  Synthetic Polypeptide
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 179 - protein, 128 aa, Artificial Sequence
  Synthetic Polypeptide
  misc_feature (108)..(108): Xaa is mFSY
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTXVTSSGAFQYWGQGTQVTVSS

SEQ ID NO: 180 - protein, 58 aa, Artificial Sequence
  Synthetic Polypeptide
  misc_feature (37)..(37): Xaa is FSY
VDNKFNKEMRNAYWEIALLPNLNNQQKRAFIRSLYDXPSQSANLLAEAKKLNDAQAPK

SEQ ID NO: 181 - protein, 15 aa, Artificial Sequence
  Synthetic Polypeptide
  misc_feature (11)..(11): Xaa is mFSY
AAGSAWYGTLXEYDY

SEQ ID NO: 182 - protein, 15 aa, Artificial Sequence
  Synthetic Polypeptide
  misc_feature (15)..(15): Xaa is mFSY
AAGSAWYGTLYEYDX

SEQ ID NO: 183 - protein, 124 aa, Artificial Sequence; identical to SEQ ID NO: 160
  Synthetic Polypeptide
  misc_feature (109)..(109): Xaa is mFSY

SEQ ID NO: 184 - protein, 124 aa, Artificial Sequence; identical to SEQ ID NO: 161
  Synthetic Polypeptide
  misc_feature (113)..(113): Xaa is mFSY

SEQ ID NO: 185 - protein, 128 aa, Artificial Sequence
  Synthetic Polypeptide
  misc_feature (115)..(115): Xaa is FSY
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYXYWGQGTQVTVSSK

SEQ ID NO: 186 - protein, 128 aa, Artificial Sequence
  Synthetic Polypeptide
  misc_feature (116)..(116): Xaa is FSY
QVQLVESGGGLMQAGGSLRLSCAVSGRTFSTAAMGWFRQAPGKEREFVAAIRWSGGSAYYADSVKGRFTISRDKAKNTVYLQMNSLKYEDTAVYYCARTENVRSLLSDYATWPYDXWGQGTQVTVSSK

SEQ ID NO: 187 - protein, 140 aa, Artificial Sequence
  Synthetic Polypeptide
  misc_feature (101)..(101): Xaa is FSY
QVQLVESGGGLVQAGGSLRLSCAASGFPVEVWRMEWYRQAPGKEREGVAAIESYGHGTRYADSVKGRFTISRDNAKNTVYLQMNSLKPEDTAVYYCNVYDXGQLAYHYDYWGQGTQVTVSAGRAGEQKLISEEDLNSAVD

SEQ ID NO: 188 - protein, 134 aa, Artificial Sequence
  Synthetic Polypeptide
  misc_feature (54)..(54): Xaa is FSY
QVQLVESGGGLVQAGGSLRLSCAASGFPVYSWNMWWYRQAPGKEREWVAAIESXGDSTRYADSVKGRFTISRDNAKNTVYLQMNSLKPEDTAVYYCYVWVGHTYYGQGTQVTVSAGRAGEQKLISEEDLNSAVD

SEQ ID NO: 189 - protein, 133 aa, Artificial Sequence
  Synthetic Polypeptide
  misc_feature (57)..(57): Xaa is FSY
QVQLVESGGGLVQAGGSLRLSCAASGFPVYSWNMWWYRQAPGKEREWVAAIESHGDXTRYADSVKGRFTISRDNAKNTVYLQMNSLKPEDTAVYYCYVWVGHTYYGQGQVTVSAGRAGEQKLISEEDLNSAVD

SEQ ID NO: 190 - protein, 10 aa, Artificial Sequence
  Synthetic Polypeptide
GGSGGGGGGS

SEQ ID NO: 191 - protein, 5 aa, Artificial Sequence
  Synthetic Polypeptide
GGGSG

SEQ ID NO: 192 - protein, 58 aa, Artificial Sequence
  Synthetic Polypeptide
  misc_feature (36)..(36): Xaa is FSY
VDNKFNKEMRNAYWEIALLPNLNNQQKRAFIRSLYXDPSQSANLLAEAKKLNDAQAPK

SEQ ID NO: 193 - protein, 58 aa, Artificial Sequence; identical to SEQ ID NO: 192
  Synthetic Polypeptide
  misc_feature (36)..(36): Xaa is mFSY

SEQ ID NO: 194 - protein, 58 aa, Artificial Sequence; identical to SEQ ID NO: 180
  Synthetic Polypeptide
  misc_feature (37)..(37): Xaa is mFSY

SEQ ID NO: 195 - protein, 58 aa, Artificial Sequence
  Synthetic Polypeptide
  misc_feature (36)..(36): Xaa is FSY
AEAKYAKEMRNAYWEIALLPNLTNQQKRAFIRKLYXDPSQSSELLSEAKKLNDSQAPK

SEQ ID NO: 196 - protein, 58 aa, Artificial Sequence
  Synthetic Polypeptide
  misc_feature (37)..(37): Xaa is FSY
AEAKYAKEMRNAYWEIALLPNLTNQQKRAFIRKLYDXPSQSSELLSEAKKLNDSQAPK

SEQ ID NO: 197 - protein, 58 aa, Artificial Sequence; identical to SEQ ID NO: 195
  Synthetic Polypeptide
  misc_feature (36)..(36): Xaa is mFSY

SEQ ID NO: 198 - protein, 58 aa, Artificial Sequence; identical to SEQ ID NO: 196
  Synthetic Polypeptide
  misc_feature (37)..(37): Xaa is mFSY

SEQ ID NO: 199 - protein, 124 aa, Artificial Sequence
  Synthetic Polypeptide
  misc_feature (116)..(116): Xaa is FSY
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGXGTQVTVSS

SEQ ID NO: 200 - protein, 10 aa, Artificial Sequence
  Synthetic Polypeptide
  misc_feature (10)..(10): Xaa is FSY
DPASPAYGDX

SEQ ID NO: 201 - protein, 119 aa, Artificial Sequence; identical to SEQ ID NO: 65
  Synthetic Polypeptide
  misc_feature (108)..(108): Xaa is FSY

SEQ ID NO: 202 - protein, 10 aa, Artificial Sequence
  Synthetic Polypeptide
  misc_feature (8)..(8): Xaa is FSY
DPASPAYXDY

SEQ ID NO: 203 - protein, 119 aa, Artificial Sequence
  Synthetic Polypeptide
  misc_feature (106)..(106): Xaa is FSY
QVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYXDYWGQGTQVTVSS

SEQ ID NO: 204 - protein, 10 aa, Artificial Sequence
  Synthetic Polypeptide
  misc_feature (8)..(8): Xaa is FFY
DPASPAYXDY

SEQ ID NO: 205 - protein, 119 aa, Artificial Sequence; identical to SEQ ID NO: 203
  Synthetic Polypeptide
  misc_feature (106)..(106): Xaa is FFY

SEQ ID NO: 206 - protein, 10 aa, Artificial Sequence
  Synthetic Polypeptide
  misc_feature (6)..(6): Xaa is FSY
DPASPXYGDY

SEQ ID NO: 207 - protein, 119 aa, Artificial Sequence
  Synthetic Polypeptide
  misc_feature (104)..(104): Xaa is FSY
QVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPXYGDYWGQGTQVTVSS

SEQ ID NO: 208 - protein, 10 aa, Artificial Sequence
  Synthetic Polypeptide
  misc_feature (6)..(6): Xaa is FFY
DPASPXYGDY

SEQ ID NO: 209 - protein, 119 aa, Artificial Sequence; identical to SEQ ID NO: 207
  Synthetic Polypeptide
  misc_feature (104)..(104): Xaa is FFY

SEQ ID NO: 210 - protein, 10 aa, Artificial Sequence
  Synthetic Polypeptide
  misc_feature (4)..(4): Xaa is FSY
DPAXPAYGDY

SEQ ID NO: 211 - protein, 119 aa, Artificial Sequence
  Synthetic Polypeptide
  misc_feature (102)..(102): Xaa is FSY
QVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPAXPAYGDYWGQGTQVTVSS

SEQ ID NO: 212 - protein, 10 aa, Artificial Sequence
  Synthetic Polypeptide
  misc_feature (4)..(4): Xaa is FFY
DPAXPAYGDY

SEQ ID NO: 213 - protein, 119 aa, Artificial Sequence; identical to SEQ ID NO: 211
  Synthetic Polypeptide
  misc_feature (102)..(102): Xaa is FFY

SEQ ID NO: 214 - protein, 117 aa, Artificial Sequence
  Synthetic Polypeptide
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSS

SEQ ID NO: 215 - protein, 8 aa, Artificial Sequence
  Synthetic Polypeptide
GIDLSLYR

SEQ ID NO: 216 - protein, 7 aa, Artificial Sequence
  Synthetic Polypeptide
ITDDGTS

SEQ ID NO: 217 - protein, 11 aa, Artificial Sequence
  Synthetic Polypeptide
NAETPLSPVNY

SEQ ID NO: 218 - protein, 8 aa, Artificial Sequence
  Synthetic Polypeptide
  misc_feature (1)..(1): Xaa is FSY
XIDLSLYR

SEQ ID NO: 219 - protein, 8 aa, Artificial Sequence
  Synthetic Polypeptide
  misc_feature (3)..(3): Xaa is FSY
GIXLSLYR

SEQ ID NO: 220 - protein, 8 aa, Artificial Sequence
  Synthetic Polypeptide
  misc_feature (5)..(5): Xaa is FSY
GIDLXLYR

SEQ ID NO: 221 - protein, 8 aa, Artificial Sequence
  Synthetic Polypeptide
  misc_feature (6)..(6): Xaa is FSY
GIDLSXYR

SEQ ID NO: 222 - protein, 8 aa, Artificial Sequence
  Synthetic Polypeptide
  misc_feature (8)..(8): Xaa is FSY
GIDLSLYX

SEQ ID NO: 223 - protein, 11 aa, Artificial Sequence
  Synthetic Polypeptide
  misc_feature (4)..(4): Xaa is FSY
NAEXPLSPVNY

SEQ ID NO: 224 - protein, 11 aa, Artificial Sequence
  Synthetic Polypeptide
  misc_feature (5)..(5): Xaa is FSY
NAETXLSPVNY

SEQ ID NO: 225 - protein, 11 aa, Artificial Sequence
  Synthetic Polypeptide
  misc_feature (6)..(6): Xaa is FSY
NAETPXSPVNY

SEQ ID NO: 226 - protein, 11 aa, Artificial Sequence
  Synthetic Polypeptide
  misc_feature (8)..(8): Xaa is FSY
NAETPLSXVNY

SEQ ID NO: 227 - protein, 117 aa, Artificial Sequence
  Synthetic Polypeptide
  misc_feature (26)..(26): Xaa is FSY
QVQLVQSGGGLVHPGGSLRLSCAASXIDLSLYRMRWYRQAPGKERDLVALITDDGTSYYEDSVKGRFTITRDNPSNKVFLQMNSLKPEDTAVYYCNAETPLSPVNYWGQGTQVTVSS

SEQ ID NO: 228 - protein, 117 aa, Artificial Sequence
  Synthetic Polypeptide
  misc_feature (28)..(28): Xaa is FSY
QVQLVQSGGGLVHPGGSLRLSCAASGIXLSLYRMRWYRQAPGKERDLVALITDDGTSYYEDSVKGRFTITRDNPSNKVFLQMNSLKPEDTAVYYCNAETPLSPVNYWGQGTQVTVSS

SEQ ID NO: 229 - protein, 117 aa, Artificial Sequence
  Synthetic Polypeptide
  misc_feature (30)..(30): Xaa is FSY
QVQLVQSGGGLVHPGGSLRLSCAASGIDLXLYRMRWYRQAPGKERDLVALITDDGTSYYEDSVKGRFTITRDNPSNKVFLQMNSLKPEDTAVYYCNAETPLSPVNYWGQGTQVTVSS

SEQ ID NO: 230 - protein, 117 aa, Artificial Sequence
  Synthetic Polypeptide
  misc_feature (31)..(31): Xaa is FSY
QVQLVQSGGGLVHPGGSLRLSCAASGIDLSXYRMRWYRQAPGKERDLVALITDDGTSYYEDSVKGRFTITRDNPSNKVFLQMNSLKPEDTAVYYCNAETPLSPVNYWGQGTQVTVSS

SEQ ID NO: 231 - protein, 117 aa, Artificial Sequence
  Synthetic Polypeptide
  misc_feature (33)..(33): Xaa is FSY
QVQLVQSGGGLVHPGGSLRLSCAASGIDLSLYXMRWYRQAPGKERDLVALITDDGTSYYEDSVKGRFTITRDNPSNKVFLQMNSLKPEDTAVYYCNAETPLSPVNYWGQGTQVTVSS

SEQ ID NO: 232 - protein, 117 aa, Artificial Sequence
  Synthetic Polypeptide
  misc_feature (51)..(51): Xaa is FSY
QVQLVQSGGGLVHPGGSLRLSCAASGIDLSLYRMRWYRQAPGKERDLVALXTDDGTSYYEDSVKGRFTITRDNPSNKVFLQMNSLKPEDTAVYYCNAETPLSPVNYWGQGTQVTVSS

SEQ ID NO: 233 - protein, 117 aa, Artificial Sequence
  Synthetic Polypeptide
  misc_feature (53)..(53): Xaa is FSY
QVQLVQSGGGLVHPGGSLRLSCAASGIDLSLYRMRWYRQAPGKERDLVALITXDGTSYYEDSVKGRFTITRDNPSNKVFLQMNSLKPEDTAVYYCNAETPLSPVNYWGQGTQVTVSS

SEQ ID NO: 234 - protein, 117 aa, Artificial Sequence
  Synthetic Polypeptide
  misc_feature (54)..(54): Xaa is FSY
QVQLVQSGGGLVHPGGSLRLSCAASGIDLSLYRMRWYRQAPGKERDLVALITDXGTSYYEDSVKGRFTITRDNPSNKVFLQMNSLKPEDTAVYYCNAETPLSPVNYWGQGTQVTVSS

SEQ ID NO: 235 - protein, 117 aa, Artificial Sequence
  Synthetic Polypeptide
  misc_feature (99)..(99): Xaa is FSY
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAEXPLSPVNYWGQGTQVTVSS

SEQ ID NO: 236 - protein, 117 aa, Artificial Sequence
  Synthetic Polypeptide
  misc_feature (100)..(100): Xaa is FSY
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETXLSPVNYWGQGTQVTVSS

SEQ ID NO: 237 - protein, 117 aa, Artificial Sequence
  Synthetic Polypeptide
  misc_feature (101)..(101): Xaa is FSY
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPXSPVNYWGQGTQVTVSS

SEQ ID NO: 238 - protein, 117 aa, Artificial Sequence
  Synthetic Polypeptide
  misc_feature (103)..(103): Xaa is FSY
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSXVNYWGQGTQVTVSS

SEQ ID NO: 239 - protein, 117 aa, Artificial Sequence
  Synthetic Polypeptide
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 240 - protein, 8 aa, Artificial Sequence
  Synthetic Polypeptide
GSIFGIRT

SEQ ID NO: 241 - protein, 7 aa, Artificial Sequence
  Synthetic Polypeptide
ITMDGRV

SEQ ID NO: 242 - protein, 11 aa, Artificial Sequence
  Synthetic Polypeptide
RYSGLTSREDY

SEQ ID NO: 243 - protein, 8 aa, Artificial Sequence
  Synthetic Polypeptide
  misc_feature (2)..(2): Xaa is FSY
GXIFGIRT

SEQ ID NO: 244 - protein, 8 aa, Artificial Sequence
  Synthetic Polypeptide
  misc_feature (4)..(4): Xaa is FSY
GSIXGIRT

SEQ ID NO: 245 - protein, 8 aa, Artificial Sequence
  Synthetic Polypeptide
  misc_feature (6)..(6): Xaa is FSY
GSIFGXRT

SEQ ID NO: 246 - protein, 8 aa, Artificial Sequence
  Synthetic Polypeptide
  misc_feature (7)..(7): Xaa is FSY
GSIFGIXT

SEQ ID NO: 247 - protein, 7 aa, Artificial Sequence
  Synthetic Polypeptide
  misc_feature (2)..(2): Xaa is FSY
IXMDGRV

SEQ ID NO: 248 - protein, 7 aa, Artificial Sequence
  Synthetic Polypeptide
  misc_feature (3)..(3): Xaa is FSY
ITXDGRV

SEQ ID NO: 249 - protein, 7 aa, Artificial Sequence
  Synthetic Polypeptide
  misc_feature (4)..(4): Xaa is FSY
ITMXGRV

SEQ ID NO: 250 - protein, 7 aa, Artificial Sequence
  Synthetic Polypeptide
  misc_feature (5)..(5): Xaa is FSY
ITMDXRV

SEQ ID NO: 251 - protein, 11 aa, Artificial Sequence
  Synthetic Polypeptide
  misc_feature (1)..(1): Xaa is FSY
XYSGLTSREDY

SEQ ID NO: 252 - protein, 11 aa, Artificial Sequence
  Synthetic Polypeptide
  misc_feature (6)..(6): Xaa is FSY
RYSGLXSREDY

SEQ ID NO: 253 - protein, 11 aa, Artificial Sequence
  Synthetic Polypeptide
  misc_feature (7)..(7): Xaa is FSY
RYSGLTXREDY

SEQ ID NO: 254 - protein, 11 aa, Artificial Sequence
  Synthetic Polypeptide
  misc_feature (10)..(10): Xaa is FSY
RYSGLTSREXY

SEQ ID NO: 255 - protein, 117 aa, Artificial Sequence
  Synthetic Polypeptide
  misc_feature (27)..(27): Xaa is FSY
QVQLVQSGGGLVQAGGSLRLSCAPSGXIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 256 - protein, 117 aa, Artificial Sequence
  Synthetic Polypeptide
  misc_feature (29)..(29): Xaa is FSY
QVQLVQSGGGLVQAGGSLRLSCAPSGSIXGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 257 - protein, 117 aa, Artificial Sequence
  Synthetic Polypeptide
  misc_feature (31)..(31): Xaa is FSY
QVQLVQSGGGLVQAGGSLRLSCAPSGSIFGXRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 258 - protein, 117 aa, Artificial Sequence
  Synthetic Polypeptide
  misc_feature (32)..(32): Xaa is FSY
QVQLVQSGGGLVQAGGSLRLSCAPSGSIFGIXTMDWYRQAPGKERELVARITMDGRVFHADSVKGRFSGSRDGASNAVYLQMNSLKPDDTAVYYCRYSGLTSREDYWGPGTQVTVSS

SEQ ID NO: 259 - protein, 117 aa, Artificial Sequence
  Synthetic Polypeptide
  misc_feature (52)..(52): Xaa is FSY
QVQLVQSGGGLVQAGGSLRLSCAPSGSIFGIRTMDWYRQAPGKERELVARIXMDGRVFHADSVKGRFSGSRDGASNAVYLQMNSLKPDDTAVYYCRYSGLTSREDYWGPGTQVTVSS

SEQ ID NO: 260 - protein, 117 aa, Artificial Sequence
  Synthetic Polypeptide
  misc_feature (53)..(53): Xaa is FSY
QVQLVQSGGGLVQAGGSLRLSCAPSGSIFGIRTMDWYRQAPGKERELVARITXDGRVFHADSVKGRFSGSRDGASNAVYLQMNSLKPDDTAVYYCRYSGLTSREDYWGPGTQVTVSS

SEQ ID NO: 261 - protein, 117 aa, Artificial Sequence
  Synthetic Polypeptide
  misc_feature (54)..(54): Xaa is FSY
QVQLVQSGGGLVQAGGSLRLSCAPSGSIFGIRTMDWYRQAPGKERELVARITMXGRVFHADSVKGRFSGSRDGASNAVYLQMNSLKPDDTAVYYCRYSGLTSREDYWGPGTQVTVSS

SEQ ID NO: 262 - protein, 117 aa, Artificial Sequence
  Synthetic Polypeptide
  misc_feature (55)..(55): Xaa is FSY
QVQLVQSGGGLVQAGGSLRLSCAPSGSIFGIRTMDWYRQAPGKERELVARITMDXRVFHADSVKGRFSGSRDGASNAVYLQMNSLKPDDTAVYYCRYSGLTSREDYWGPGTQVTVSS

SEQ ID NO: 263 - protein, 117 aa, Artificial Sequence
  Synthetic Polypeptide
  misc_feature (96)..(96): Xaa is FSY
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCXYSGLTSREDYWGPGTQVTVSS

SEQ ID NO: 264 - protein, 117 aa, Artificial Sequence
  Synthetic Polypeptide
  misc_feature (101)..(101): Xaa is FSY
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLXSREDYWGPGTQVTVSS

SEQ ID NO: 265 - protein, 117 aa, Artificial Sequence
  Synthetic Polypeptide
  misc_feature (102)..(102): Xaa is FSY
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTXREDYWGPGTQVTVSS

SEQ ID NO: 266 - protein, 117 aa, Artificial Sequence
  Synthetic Polypeptide
  misc_feature (105)..(105): Xaa is FSY
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREXYWGPGTQVTVSS

SEQ ID NO: 267 - protein, 134 aa, Artificial Sequence
  Synthetic Polypeptide
  misc_feature (57)..(57): Xaa is FSY
QVQLVESGGGLVQAGGSLRLSCAASGFPVYSWNMWWYRQAPGKEREWVAAIESHGDXTRYADSVKGRFTISRDNAKNTVYLQMNSLKPEDTAVYYCYVWVGHTYYGQGTQVTVSAGRAGEQKLISEEDLNSAVD

SEQ ID NO: 268 - protein, 11 aa, Artificial Sequence
  Synthetic Polypeptide
  misc_feature (8)..(8): Xaa is FSY
AIESHGDXSTR

SEQ ID NO: 269 - DNA, 40 bp, Artificial Sequence
  Synthetic Polynucleotide
aagaaggagatatacatatgaacatccccgctctggtgga

SEQ ID NO: 270 - DNA, 60 bp, Artificial Sequence
  Synthetic Polynucleotide
catccgccaaaacagccaagcttttagtgatggtgatggtgatgggcatagtcggggaca

SEQ ID NO: 271 - DNA, 18 bp, Artificial Sequence
  Synthetic Polynucleotide
tccctgtacatcttcagc

SEQ ID NO: 272 - DNA, 25 bp, Artificial Sequence
  Synthetic Polynucleotide
cctgaccaacgcctacaagacctac

SEQ ID NO: 273 - DNA, 22 bp, Artificial Sequence
  Synthetic Polynucleotide
cggatcttccgcaggatgtcgc

SEQ ID NO: 274 - DNA, 28 bp, Artificial Sequence
  Synthetic Polynucleotide
gaacgccttcgatgccaacaattacccc

SEQ ID NO: 275 - DNA, 25 bp, Artificial Sequence
  Synthetic Polynucleotide
cctgaccaactagtacaagacctac

SEQ ID NO: 276 - DNA, 28 bp, Artificial Sequence
  Synthetic Polynucleotide
gaacgccttcgattagaacaattacccc

SEQ ID NO: 277 - DNA, 35 bp, Artificial Sequence
  Synthetic Polynucleotide
tgagatacctgctggccgccgacaagacctgcatc

SEQ ID NO: 278 - DNA, 32 bp, Artificial Sequence
  Synthetic Polynucleotide
ggccagcaggtatctcagcttgcccatgttca

SEQ ID NO: 279 - DNA, 34 bp, Artificial Sequence
  Synthetic Polynucleotide
agatacctgctgtaggccgacaagacctgcatcg

SEQ ID NO: 280 - DNA, 32 bp, Artificial Sequence
  Synthetic Polynucleotide
gcctacagcaggtatctcagcttgcccatgtt

SEQ ID NO: 281 - DNA, 32 bp, Artificial Sequence
  Synthetic Polynucleotide
gacgccacctgcatcgacggccagaccagagt

SEQ ID NO: 282 - DNA, 35 bp, Artificial Sequence
  Synthetic Polynucleotide
tcgatgcaggtggcgtcggccttcagcaggtatct

SEQ ID NO: 283 - DNA, 32 bp, Artificial Sequence
  Synthetic Polynucleotide
gactagacctgcatcgacggccagaccagagt

SEQ ID NO: 284 - DNA, 35 bp, Artificial Sequence
  Synthetic Polynucleotide
tcgatgcaggtctagtcggccttcagcaggtatct

SEQ ID NO: 285 - DNA, 32 bp, Artificial Sequence
  Synthetic Polynucleotide
accgccgtcagagtgatcgagcagcccctgaa

SEQ ID NO: 286 - DNA, 32 bp, Artificial Sequence
  Synthetic Polynucleotide
atcactctgacggcggtctggccgtcgatgca

SEQ ID NO: 287 - DNA, 32 bp, Artificial Sequence
  Synthetic Polynucleotide
cagacctaggtcagagtgatcgagcagcccct

SEQ ID NO: 288 - DNA, 32 bp, Artificial Sequence
  Synthetic Polynucleotide
actctgacctaggtctggccgtcgatgcaggt

SEQ ID NO: 289 - DNA, 32 bp, Artificial Sequence
  Synthetic Polynucleotide
agagtcgccgtgatcgagcagcccctgaacgg

SEQ ID NO: 290 - DNA, 32 bp, Artificial Sequence
  Synthetic Polynucleotide
tcgatcacggcgactctggtctggccgtcgat

SEQ ID NO: 291 - DNA, 32 bp, Artificial Sequence
  Synthetic Polynucleotide
cagagtctaggtgatcgagcagcccctgaacg

SEQ ID NO: 292 - DNA, 32 bp, Artificial Sequence
  Synthetic Polynucleotide
cgatcacctagactctggtctggccgtcgatg

SEQ ID NO: 293 - DNA, 58 bp, Artificial Sequence
  Synthetic Polynucleotide
aataatacgactcactatatagattgctgttctaccaagtaatccatgttgtggaagg

SEQ ID NO: 294 - DNA, 50 bp, Artificial Sequence
  Synthetic Polynucleotide
tgttgtaatagcccccaaaactggaccttccacaacatggattacttggt

SEQ ID NO: 295 - DNA, 60 bp, Artificial Sequence
  Synthetic Polynucleotide
taataatacgactcactatagggggccagtgaattcgagctcggtacccggggatcctct

SEQ ID NO: 296 - DNA, 60 bp, Artificial Sequence
  Synthetic Polynucleotide
gtcgagtagattgctgttctaccaagtaatccatatttctagaggatccccgggtaccga

SEQ ID NO: 297 - DNA, 60 bp, Artificial Sequence
  Synthetic Polynucleotide
cgaggtgcctttactacatgtgtgatctgataaccttttgttgtaatagcccccaaaact

SEQ ID NO: 298 - DNA, 60 bp, Artificial Sequence
  Synthetic Polynucleotide
cgaggtgcctttactacatgtgtgatctgataacctggggttgtaatagcccccaaaact

SEQ ID NO: 299 - DNA, 60 bp, Artificial Sequence
  Synthetic Polynucleotide
cgaggtgcctttactacatgtgtgatctgataacctcccgttgtaatagcccccaaaact

SEQ ID NO: 300 - DNA, 60 bp, Artificial Sequence
  Synthetic Polynucleotide
cgaggtgcctttactacatgtgtgatctgataacctaaagttgtaatagcccccaaaact

SEQ ID NO: 301 - DNA, 33 bp, Artificial Sequence
  Synthetic Polynucleotide
cccatatggctaaggggcaatctttacaagatc

SEQ ID NO: 302 - DNA, 53 bp, Artificial Sequence
  Synthetic Polynucleotide
ggaagcttagtgatggtgatggtgatgttcggtttcttcgctgtcctgttgcg

SEQ ID NO: 303 - DNA, 34 bp, Artificial Sequence
  Synthetic Polynucleotide
agtttctatttagttggtgaatggtattaagctg

SEQ ID NO: 304 - DNA, 18 bp, Artificial Sequence
  Synthetic Polynucleotide
ggaacacgttcccgacgc

SEQ ID NO: 305 - DNA, 23 bp, Artificial Sequence
  Synthetic Polynucleotide
ggtgaatggttagaagctgcaag

SEQ ID NO: 306 - DNA, 22 bp, Artificial Sequence
  Synthetic Polynucleotide
aaataaatagaaactggaacac

SEQ ID NO: 307 - DNA, 25 bp, Artificial Sequence
  Synthetic Polynucleotide
gttgaaaaactaggtcagccagatg

SEQ ID NO: 308 - DNA, 22 bp, Artificial Sequence
  Synthetic Polynucleotide
aggatcacgaactgatcaaaag

SEQ ID NO: 309 - DNA, 19 bp, Artificial Sequence
  Synthetic Polynucleotide
cgggcggaggggaggaaag

SEQ ID NO: 310 - DNA, 22 bp, Artificial Sequence
  Synthetic Polynucleotide
atcggcggtttgctctctgttg

SEQ ID NO: 311 - DNA, 19 bp, Artificial Sequence
  Synthetic Polynucleotide
catcctggccgatgaaaaa

SEQ ID NO: 312 - DNA, 23 bp, Artificial Sequence
  Synthetic Polynucleotide
ttgacgatgctctgcttcatatc

SEQ ID NO: 313 - DNA, 22 bp, Artificial Sequence
  Synthetic Polynucleotide
ctccgttctcatcaaattccgc

SEQ ID NO: 314 - DNA, 15 bp, Artificial Sequence
  Synthetic Polynucleotide
gacggaaccgcctgc

SEQ ID NO: 315 - DNA, 32 bp, Artificial Sequence
  Synthetic Polynucleotide
  misc_feature (1)..(10): n is a, c, g, or t
nnnnnnnnnnagatcggaagagcacacgtctg

SEQ ID NO: 316 - DNA, 38 bp, Artificial Sequence
  Synthetic Polynucleotide
caaaacagccaagcttcagacgtgtgctcttccgatct

SEQ ID NO: 317 - DNA, 46 bp, Artificial Sequence
  Synthetic Polynucleotide
aagaaggagatatacatcagcgtattctgactcataaggtggctcc

SEQ ID NO: 318 - DNA, 35 bp, Artificial Sequence
  Synthetic Polynucleotide
aagaaggagatatacatctgcgataggcagtacgg

SEQ ID NO: 319 - DNA, 58 bp, Artificial Sequence
  Synthetic Polynucleotide
gctggctagcgtttaaacttaagctgccaccatgaacatccccgctctggtggaaaac

SEQ ID NO: 320 - DNA, 56 bp, Artificial Sequence
  Synthetic Polynucleotide
ttccaccacactggactagtggatcttagtgatggtgatggtgatgggcatagtcg

SEQ ID NO: 321 - DNA, 60 bp, Artificial Sequence
  Synthetic Polynucleotide
aaaggacgaaacaccctggcggcgggtgtggacgggcggcggatcgttgtggaaggtcca

SEQ ID NO: 322 - DNA, 60 bp, Artificial Sequence
  Synthetic Polynucleotide
aaaggacgaaacaccggttttcagatcacacatgtagtaaaggcagttgtggaaggtcca

SEQ ID NO: 323 - DNA, 60 bp, Artificial Sequence
  Synthetic Polynucleotide
aaaggacgaaacaccaattgtttgcatcatccccaagtcattggtgttgtggaaggtcca

SEQ ID NO: 324 - DNA, 59 bp, Artificial Sequence
  Synthetic Polynucleotide
aattcgagctcggtaccaaaaaagttgtaatagcccctcaaaactggaccttccacaac

SEQ ID NO: 325 - DNA, 19 bp, Artificial Sequence
  Synthetic Polynucleotide
ctgggctacactgagcacc

SEQ ID NO: 326 - DNA, 21 bp, Artificial Sequence
  Synthetic Polynucleotide
aagtggtcgttgagggcaatg

SEQ ID NO: 327 - DNA, 20 bp, Artificial Sequence
  Synthetic Polynucleotide
ctggcaccacaccttctaca

SEQ ID NO: 328 - DNA, 20 bp, Artificial Sequence
  Synthetic Polynucleotide
gaggcgtacagggatagcac

SEQ ID NO: 329 - DNA, 22 bp, Artificial Sequence
  Synthetic Polynucleotide
tcctcctggtggccaagacagc

SEQ ID NO: 330 - DNA, 22 bp, Artificial Sequence
  Synthetic Polynucleotide
gctaaggggcagcgaaggatgc

SEQ ID NO: 331 - DNA, 37 bp, Artificial Sequence
  Synthetic Polynucleotide
aacaggaggaattactagtatggataaaaagcctttg

SEQ ID NO: 332 - DNA, 38 bp, Artificial Sequence
  Synthetic Polynucleotide
gatgatgatgatgatggtcgacttacaggttagtagaa

SEQ ID NO: 333 - DNA, 35 bp, Artificial Sequence
  Synthetic Polynucleotide
taacaggaggaattactagtatgaccgtgaagtac

SEQ ID NO: 334 - DNA, 16 bp, Artificial Sequence
  Synthetic Polynucleotide
caggtttggcgccagc

SEQ ID NO: 335 - DNA, 39 bp, Artificial Sequence
  Synthetic Polynucleotide
gctggcgccaaacctgttgagcgtggctcgtgacctgcg

SEQ ID NO: 336 - DNA, 16 bp, Artificial Sequence
  Synthetic Polynucleotide
cagcatggtgaactcc

SEQ ID NO: 337 - DNA, 38 bp, Artificial Sequence
  Synthetic Polynucleotide
ggagttcaccatgctggctctgatggatatgggtccgc

SEQ ID NO: 338 - DNA, 15 bp, Artificial Sequence
  Synthetic Polynucleotide
cggctcatgcacgtc

SEQ ID NO: 339 - DNA, 32 bp, Artificial Sequence
  Synthetic Polynucleotide
cgtgcatgagccgacaagcggtgctggttttg

SEQ ID NO: 340 - DNA, 39 bp, Artificial Sequence
  Synthetic Polynucleotide
tgatgatgatgatggtcgacttaattgattttggcacca

SEQ ID NO: 341 - DNA, 53 bp, Artificial Sequence
  Synthetic Polynucleotide
ctagcatagcggggttcgacgccccggtctctcgccaaattcgaaaagcctgc

SEQ ID NO: 342 - DNA, 51 bp, Artificial Sequence
  Synthetic Polynucleotide
gttttagagacccgctggtcgccggaccgtcccccaatgcggggcgcatct

SEQ ID NO: 343 - DNA, 25 bp, Artificial Sequence
  Synthetic Polynucleotide
aaaacctagccagcggggttcgacg

SEQ ID NO: 344 - DNA, 18 bp, Artificial Sequence
  Synthetic Polynucleotide
agagacccgctggtcgcc

SEQ ID NO: 345 - DNA, 39 bp, Artificial Sequence
  Synthetic Polynucleotide
gggagacccaagctggctagcgccaccatggataaaaag

SEQ ID NO: 346 - DNA, 42 bp, Artificial Sequence
  Synthetic Polynucleotide
ctgatcagcgggtttaaagcggccgcttacaggttagtagaa

SEQ ID NO: 347 - DNA, 17 bp, Artificial Sequence
  Synthetic Polynucleotide
gggcaggaagagggcct

SEQ ID NO: 348 - DNA, 32 bp, Artificial Sequence
  Synthetic Polynucleotide
cgccggaccgtcccccggtgtttcgtcctttc

SEQ ID NO: 349 - DNA, 32 bp, Artificial Sequence
  Synthetic Polynucleotide
gaaaggacgaaacaccgggggacggtccggcg

SEQ ID NO: 350 - DNA, 37 bp, Artificial Sequence
  Synthetic Polynucleotide
ctcttcctgcccctcgacaaaaaacgagagaccgggg

SEQ ID NO: 351 - DNA, 39 bp, Artificial Sequence
  Synthetic Polynucleotide
ggggatcgggtctagactcgaggggcaggaagagggcct

SEQ ID NO: 352 - DNA, 37 bp, Artificial Sequence
  Synthetic Polynucleotide
gtgccacctgacgtcgacaaaaaacgagagaccgggg

SEQ ID NO: 353 - DNA, 37 bp, Artificial Sequence
  Synthetic Polynucleotide
gagacccaagctggctagcgccaccatgaccgtgaag

SEQ ID NO: 354 - DNA, 41 bp, Artificial Sequence
  Synthetic Polynucleotide
gatcagcgggtttaaagcggccgcttaattgattttggcac

SEQ ID NO: 355 - DNA, 50 bp, Artificial Sequence
  Synthetic Polynucleotide
gctggctagcgtttaaacttaagctgccaccatgtcggccaccagcgtgg

SEQ ID NO: 356 - DNA, 46 bp, Artificial Sequence
  Synthetic Polynucleotide
tgagggtgagaccacgcactttgtttgtttcgactctgccgttcct

SEQ ID NO: 357 - DNA, 60 bp, Artificial Sequence
  Synthetic Polynucleotide
agtgcgtggtctcaccctcaatttgagaagggtggtgggtcaggtgggggttcaggggga

SEQ ID NO: 358 - DNA, 60 bp, Artificial Sequence
  Synthetic Polynucleotide
cccgccacccttttcaaactggggatgactccatgctgatccccctgaacccccacctga

SEQ ID NO: 359 - DNA, 77 bp, Artificial Sequence
  Synthetic Polynucleotide
agtttgaaaagggtggcgggtacccatacgatgttccagattacgcttaagatccactagtccagtgtggtggaatt

SEQ ID NO: 360 - DNA, 47 bp, Artificial Sequence
  Synthetic Polynucleotide
tttaaacgctagccagcttgggtctccctatagtgagtcgtattaat

SEQ ID NO: 361 - DNA, 32 bp, Artificial Sequence
  Synthetic Polynucleotide
caagagctagtctgaggacgacatccaccgct

SEQ ID NO: 362 - DNA, 32 bp, Artificial Sequence
  Synthetic Polynucleotide
cctcagactagctcttgatgatgaacacacgc

SEQ ID NO: 363 - DNA, 18 bp, Artificial Sequence
  Synthetic Polynucleotide
caatcaaagtcctcggcc

SEQ ID NO: 364 - DNA, 21 bp, Artificial Sequence
  Synthetic Polynucleotide
gaaaggttcttttgttggctg

SEQ ID NO: 365 - DNA, 21 bp, Artificial Sequence
  Synthetic Polynucleotide
gagttcatctgtaggctcagc

SEQ ID NO: 366 - DNA, 47 bp, Artificial Sequence
  Synthetic Polynucleotide
aagaaggagatatacatccgttccagtttttaaatcctgagtcaagc

SEQ ID NO: 367 - DNA, 38 bp, Artificial Sequence
  Synthetic Polynucleotide
aagaaggagatatacatgggccttttcccgatcagtcc

SEQ ID NO: 368 - DNA, 53 bp, Artificial Sequence
  Synthetic Polynucleotide
aagaaggagatatacatgagtactacagaagcaatctacagtctctattgcag

SEQ ID NO: 369 - DNA, 36 bp, Artificial Sequence
  Synthetic Polynucleotide
ctaacaggaggaattagatctatggataaaaagcct

SEQ ID NO: 370 - DNA, 38 bp, Artificial Sequence; identical to SEQ ID NO: 332
  Synthetic Polynucleotide

SEQ ID NO: 371 - DNA, 24 bp, Artificial Sequence
  Synthetic Polynucleotide
tatgccatggataaaaagcctttg

SEQ ID NO: 372 - DNA, 25 bp, Artificial Sequence
  Synthetic Polynucleotide
ctatgctagcttacaggttagtaga

SEQ ID NO: 373 - DNA, 36 bp, Artificial Sequence
  Synthetic Polynucleotide
tcttgtggtatgggttggtagcgtcagagcccgggt

SEQ ID NO: 374 - DNA, 35 bp, Artificial Sequence
  Synthetic Polynucleotide
ctaccaacccataccacaagagttgaagatatagc

SEQ ID NO: 375 - DNA, 32 bp, Artificial Sequence
  Synthetic Polynucleotide
actaggcatcctttctctactctggagtccct

SEQ ID NO: 376 - DNA, 39 bp, Artificial Sequence
  Synthetic Polynucleotide
gagaaaggatgcctagtaaatcagaagcttcggagcttt

SEQ ID NO: 377 - DNA, 23 bp, Artificial Sequence
  Synthetic Polynucleotide
tcagcaacattagaccacaccac

SEQ ID NO: 378 - DNA, 23 bp, Artificial Sequence
  Synthetic Polynucleotide
cagtaataagttgcgaagtcttc

SEQ ID NO: 379 - DNA, 33 bp, Artificial Sequence
  Synthetic Polynucleotide
atgaatacgactactggggttagggtacgcagg

SEQ ID NO: 380 - DNA, 33 bp, Artificial Sequence
  Synthetic Polynucleotide
aaccccagtagtcgtattcatacagagtgccat

SEQ ID NO: 381 - DNA, 36 bp, Artificial Sequence
  Synthetic Polynucleotide
tgtgatgtagagtttctataagcaccttggaattga

SEQ ID NO: 382 - DNA, 36 bp, Artificial Sequence
  Synthetic Polynucleotide
agaaactctacatcacataattttgacaacgatcac

SEQ ID NO: 383 - DNA, 40 bp, Artificial Sequence
  Synthetic Polynucleotide
taagaaggagatatacatatgaaatatctgctgccaaccg

SEQ ID NO: 384 - DNA, 41 bp, Artificial Sequence
  Synthetic Polynucleotide
gccaaaacagccaagcttttaatgatggtgatggtggtgtt

SEQ ID NO: 385 - DNA, 23 bp, Artificial Sequence
  Synthetic Polynucleotide
  misc_feature (11)..(11): n is TAG
ggttagcggtnacctttagcacc

SEQ ID NO: 386 - DNA, 18 bp, Artificial Sequence
  Synthetic Polynucleotide
gcgcaactcagacgcaga

SEQ ID NO: 387 - DNA, 28 bp, Artificial Sequence
  Synthetic Polynucleotide
  misc_feature (11)..(11): n is TAG
tcgcacctttnaccgccgcgatgggttg

SEQ ID NO: 388 - DNA, 18 bp, Artificial Sequence
  Synthetic Polynucleotide
ccgctaaccgcgcaactc

SEQ ID NO: 389 - DNA, 21 bp, Artificial Sequence
  Synthetic Polynucleotide
  misc_feature (11)..(11): n is TAG
cgcgcgcaccnaacgttcgta

SEQ ID NO: 390 - DNA, 18 bp, Artificial Sequence
  Synthetic Polynucleotide
caatagtacacggcggtg

SEQ ID NO: 391 - DNA, 21 bp, Artificial Sequence
  Synthetic Polynucleotide
  misc_feature (11)..(11): n is TAG
ttacgccacgnccgtacgatt

SEQ ID NO: 392 - DNA, 20 bp, Artificial Sequence
  Synthetic Polynucleotide
tcgctcagcagactacgaac

SEQ ID NO: 393 - DNA, 21 bp, Artificial Sequence
  Synthetic Polynucleotide
  misc_feature (11)..(11): n is TAG
gtggccgtacntactggggtc

SEQ ID NO: 394 - DNA, 18 bp, Artificial Sequence
  Synthetic Polynucleotide
gtggcgtaatcgctcagc

SEQ ID NO: 395 - DNA, 21 bp, Artificial Sequence
  Synthetic Polynucleotide
  misc_feature (11)..(11): n is TAG
gccgtacgatntggggtcaag

SEQ ID NO: 396 - DNA, 18 bp, Artificial Sequence
  Synthetic Polynucleotide
cacgtggcgtaatcgctc

SEQ ID NO: 397 - DNA, 43 bp, Artificial Sequence
  Synthetic Polynucleotide
ctttaagaaggagatatacatatgaaatatctgctgccaaccg

SEQ ID NO: 398 - DNA, 40 bp, Artificial Sequence
  Synthetic Polynucleotide
tccgccaaaacagccaagcttttaatggtgatgatgatgg

SEQ ID NO: 399 - DNA, 21 bp, Artificial Sequence
  Synthetic Polynucleotide
  misc_feature (11)..(11): n is TAG
ctgcaacgtgngatgatggcc

SEQ ID NO: 400 - DNA, 18 bp, Artificial Sequence
  Synthetic Polynucleotide
tagtacacggccgtatcc

SEQ ID NO: 401 - DNA, 21 bp, Artificial Sequence
  Synthetic Polynucleotide
  misc_feature (11)..(11): n is TAG
cgtgtacgatnggccagctgg

SEQ ID NO: 402 - DNA, 18 bp, Artificial Sequence
  Synthetic Polynucleotide
ttgcagtagtacacggcc

SEQ ID NO: 403 - DNA, 44 bp, Artificial Sequence
  Synthetic Polynucleotide
ctttaagaaggagatatacatatgaaatatctgctgccaaccgc

SEQ ID NO: 404 - DNA, 41 bp, Artificial Sequence
  Synthetic Polynucleotide
atccgccaaaacagccaagcttttaatgatgatggtgatgg

SEQ ID NO: 405 - DNA, 21 bp, Artificial Sequence
  Synthetic Polynucleotide
  misc_feature (11)..(11): n is TAG
catgtggtggncgccaagccc

SEQ ID NO: 406 - DNA, 23 bp, Artificial Sequence
  Synthetic Polynucleotide
ttccagctgtacactggaaagcc

SEQ ID NO: 407 - DNA, 24 bp, Artificial Sequence
  Synthetic Polynucleotide
  misc_feature (11)..(11): n is TAG
gatcgaaagcnggcgatagcaccc

SEQ ID NO: 408 - DNA, 18 bp, Artificial Sequence
  Synthetic Polynucleotide
gccgcaacccattcgcgt

SEQ ID NO: 409 - DNA, 23 bp, Artificial Sequence
  Synthetic Polynucleotide
  misc_feature (11)..(11): n is TAG
ccacggcgatnacccgctacgcg

SEQ ID NO: 410 - DNA, 18 bp, Artificial Sequence
  Synthetic Polynucleotide
ctttcgatcgccgcaacc

SEQ ID NO: 411 - DNA, 48 bp, Artificial Sequence; identical to SEQ ID NO: 6
  Synthetic Polynucleotide

SEQ ID NO: 412 - DNA, 57 bp, Artificial Sequence; identical to SEQ ID NO: 7
  Synthetic Polynucleotide

SEQ ID NO: 413 - DNA, 28 bp, Artificial Sequence
  Synthetic Polynucleotide
  misc_feature (15)..(15): n is TAG
ccaagacatttttgnaagtttaaccacg

SEQ ID NO: 414 - DNA, 27 bp, Artificial Sequence
  Synthetic Polynucleotide
ctacaaaaatgtcttggcctgttcctc

SEQ ID NO: 415 - DNA, 34 bp, Artificial Sequence
  Synthetic Polynucleotide
  misc_feature (20)..(20): n is TAG
gtttaaccacgaagccgaanctgttctatcaaag

SEQ ID NO: 416 - DNA, 25 bp, Artificial Sequence
  Synthetic Polynucleotide
ctattcggcttcgtggttaaacttg

SEQ ID NO: 417 - DNA, 37 bp, Artificial Sequence
  Synthetic Polynucleotide
  misc_feature (20)..(20): n is TAG
caagtttaaccacgaagccngacctgttctatcaaag

SEQ ID NO: 418 - DNA, 24 bp, Artificial Sequence
  Synthetic Polynucleotide
ctaggcttcgtggttaaacttgtc

SEQ ID NO: 419 - DNA, 36 bp, Artificial Sequence
  Synthetic Polynucleotide
  misc_feature (21)..(21): n is a, c, g, or t
  misc_feature (31)..(31): n is TAG
catttttggacaagtttaacngaagccgaagacctg

SEQ ID NO: 420 - DNA, 29 bp, Artificial Sequence
  Synthetic Polynucleotide
ggcttcctagttaaacttgtccaaaaatg

SEQ ID NO: 421 - DNA, 31 bp, Artificial Sequence
  Synthetic Polynucleotide
  misc_feature (17)..(17): n is TAG
cgaagacctgttctatnagttcacttgcttc

SEQ ID NO: 422 - DNA, 29 bp, Artificial Sequence
  Synthetic Polynucleotide
ctctaatagaacaggtcttcggcttcgtg

SEQ ID NO: 423 - DNA, 21 bp, Artificial Sequence
  Synthetic Polynucleotide
  misc_feature (11)..(11): n is TAG
tgcccaaatgnccactacaag

SEQ ID NO: 424 - DNA, 24 bp, Artificial Sequence
  Synthetic Polynucleotide
agtgtggactgttcctttaaaaag

SEQ ID NO: 425 - protein, 36 aa, Artificial Sequence
  Synthetic Polypeptide
TLRFQVHLGNYLHDSRPKENLISDRRIKEKITVFGR

SEQ ID NO: 426 - protein, 40 aa, Artificial Sequence
  Synthetic Polypeptide
HIRFHVNMGKLRYLIKADKTCIDGQTRVRVIEQPLNGFGR

SEQ ID NO: 427 - protein, 38 aa, Artificial Sequence
  Synthetic Polypeptide
SIRFQVDLGTYHYCIYDKKIGDEQERRHLTRTLLSFGR

SEQ ID NO: 428 - protein, 38 aa, Artificial Sequence
  Synthetic Polypeptide
SLRFHIDLGTYHFAIYKKMIGEQPEDRHLTRNLYGFGR

SEQ ID NO: 429 - protein, 38 aa, Artificial Sequence
  Synthetic Polypeptide
QLRFQIDLGTYHFSIYNRKIGDEDEVRHLTHHLYGFAR

SEQ ID NO: 430 - DNA, 36 bp, Artificial Sequence
  Synthetic Polynucleotide
cgggaacaacaagaagttaaggcggggcaaaaaata

SEQ ID NO: 431 - DNA, 50 bp, Artificial Sequence
  Synthetic Polynucleotide
gacgtgtgctcttccgatctgcccttgcgcgttaaggcggggcaaaaaat

SEQ ID NO: 432 - DNA, 50 bp, Artificial Sequence
  Synthetic Polynucleotide
gacgtgtgctcttccgatctgaccccatgtggcggggcaaaaaatagcga

SEQ ID NO: 433 - DNA, 46 bp, Artificial Sequence
  Synthetic Polynucleotide
gtgtgcacgagtgggaaggacttggcgcgccctcccttggcgtgga

SEQ ID NO: 434 - DNA, 50 bp, Artificial Sequence
  Synthetic Polynucleotide
gacgtgtgctcttccgatcttcaccccgtaggacttggcgcgccctccct

SEQ ID NO: 435 - DNA, 49 bp, Artificial Sequence
  Synthetic Polynucleotide
cctgcgggctgccccgctttgcggacgggctgtccccgcgcaacggaac

SEQ ID NO: 436 - DNA, 46 bp, Artificial Sequence
  Synthetic Polynucleotide
acgtgtgctcttccgatctcccagccgaagacgggctgtccccgcg

SEQ ID NO: 437 - protein, 47 aa, Artificial Sequence
  Synthetic Polypeptide
DSVFRELGKRLKGKEYTSENFFDAIFKENISLVEYERTVKLLSDYFP

SEQ ID NO: 438 - protein, 59 aa, Artificial Sequence
  Synthetic Polypeptide
QTVLDHIQKVADIEGEQNENNENLWFHPVMSHLYNAKNGYDKQPEKTMFIIERLQSYFP

SEQ ID NO: 439 - protein, 55 aa, Artificial Sequence
  Synthetic Polypeptide
FITICHINEQLGLKTPSNDDKIVDVVCETWNNILNNDHDLLKKSQLTELILKHFP

SEQ ID NO: 440 - protein, 53 aa, Artificial Sequence
  Synthetic Polypeptide
YITLTHIDRQLAYSKADITNDQDVLSFKALWKNFDNDLERKSRLRSLILKHFS

SEQ ID NO: 441 - protein, 51 aa, Artificial Sequence
  Synthetic Polypeptide
YTTINHINRRLEIAELKDDGYMMGIKGSWNEQAKKLDKKVRLRDLIMKHFP

SEQ ID NO: 442 - protein, 34 aa, Artificial Sequence
  Synthetic Polypeptide
MARLLDKKEVPIKERKENFKKNFKGIIKAVRDLR

SEQ ID NO: 443 - protein, 44 aa, Artificial Sequence
  Synthetic Polypeptide
FLKIMAENQREYSNGKYKQNRVEVNSNDIFEVLKRAFGVLKMYR

SEQ ID NO: 444 - protein, 58 aa, Artificial Sequence
  Synthetic Polypeptide
FLTAMCYHPPKKEGKKKGHQKEQQKEKESEAQSQAEALNPSKLIEALEILVNQLHSLR

SEQ ID NO: 445 - protein, 60 aa, Artificial Sequence
  Synthetic Polypeptide
FLEGAAYGKKLFESKSSGNKSSKNKELTKKEKEELQANALSLDNLKSILFDFLQKLKDFR

SEQ ID NO: 446 - protein, 49 aa, Artificial Sequence
  Synthetic Polypeptide
FLEAAAYEMTNSKSPNNKEQREKEQSEALSLNNLKNVLFIFLEKLQVLR

SEQ ID NO: 447 - protein, 50 aa, Artificial Sequence
  Synthetic Polypeptide
NFYTHKEKGEVEITDEIFGVLDEMLKSTVLTVKKKKVKTDKTKEILKKSI

SEQ ID NO: 448 - protein, 49 aa, Artificial Sequence
  Synthetic Polypeptide
DLTNHYKTYEEKLNDGCEFLTSTEQPLSGMINNYYTVALRNMNERYGYK

SEQ ID NO: 449 - protein, 39 aa, Artificial Sequence
  Synthetic Polypeptide
NYYSHYKHKKPDAEKDIFKHLYKAFDASLRMVKEDYKAH

SEQ ID NO: 450 - protein, 43 aa, Artificial Sequence
  Synthetic Polypeptide
NYYSHYRHSGSSELPLFDGNMLQRLYNVFDVSVQRVKIDHEHN

SEQ ID NO: 451 - protein, 43 aa, Artificial Sequence
  Synthetic Polypeptide
NYYSHYKYSEESPKPIFETSLLKNMYKVFDANVRLVKRDYMHH

SEQ ID NO: 452 - protein, 60 aa, Artificial Sequence
  Synthetic Polypeptide
EKQLDILCQKKLEYLRDTARKIEEKRRNQRERGEKELVAPFKYSDKRDDLIAAIYNDAFD

SEQ ID NO: 453 - protein, 31 aa, Artificial Sequence
  Synthetic Polypeptide
TEDLAFIQDKRFKFVKDAYGKKKSQVNTGFF

SEQ ID NO: 454 - protein, 26 aa, Artificial Sequence
  Synthetic Polypeptide
FTVNLTRDFAHLNRKGKNKQDNPDFN

SEQ ID NO: 455 - protein, 30 aa, Artificial Sequence
  Synthetic Polypeptide
DEVDPHYHFNHLVRKGKKDRYGHNDNPSFK

SEQ ID NO: 456 - protein, 31 aa, Artificial Sequence
  Synthetic Polypeptide
ENIDMQRDFTHLNRKKQVGRTKNIIDSPNFH

SEQ ID NO: 457 - protein, 60 aa, Artificial Sequence
  Synthetic Polypeptide
VYIDKKKDSLKKESSKAKYNTKSDPQQEEGDLKIPISKNGVVFLLSLFTKQEIHAFKSKI

SEQ ID NO: 458 - protein, 41 aa, Artificial Sequence
  Synthetic Polypeptide
LSLQDYNGDTQKKLHLSGVGIALLICLFLDKQYINIFLSRL

SEQ ID NO: 459 - protein, 35 aa, Artificial Sequence
  Synthetic Polypeptide
RYRFEKDGFFTESGLLFFTNLFLDKRDAYWMLKKV

SEQ ID NO: 460 - protein, 35 aa, Artificial Sequence
  Synthetic Polypeptide
HHFVDGEGMVTEAGLLFFVSLFLEKRDAIWMQKKI

SEQ ID NO: 461 - protein, 35 aa, Artificial Sequence
  Synthetic Polypeptide
YHFADKEGNMTIAGLLFFVSLFLDKKDAIWMQKKL

SEQ ID NO: 462 - protein, 60 aa, Artificial Sequence
  Synthetic Polypeptide
AGFKATVIDEATVSEATVSHGKNSICFMATHEIFSHLAYKKLKRKVRTAEINYGEAENAE

SEQ ID NO: 463 - protein, 31 aa, Artificial Sequence
  Synthetic Polypeptide
PIFSSYNAQSEERRIIIRSFGINSIKLPKDR

SEQ ID NO: 464 - protein, 29 aa, Artificial Sequence
  Synthetic Polypeptide
SGFKASHKQRERMTTEVFCRSRILLPKLR

SEQ ID NO: 465 - protein, 29 aa, Artificial Sequence
  Synthetic Polypeptide
RGFKGGTETYQQMTNEVFCRSRISLPKLK

SEQ ID NO: 466 - protein, 29 aa, Artificial Sequence
  Synthetic Polypeptide
KGFKDGRNLREQMTNEVFCRSRISLPKLK

SEQ ID NO: 467 - protein, 62 aa, Artificial Sequence
  Synthetic Polypeptide
QLSVYAKETLMMQMLDELSKVPDVVYQNLSEDVQKTFIENDWNEYLKKENNGDVGTMEEEQV

SEQ ID NO: 468 - protein, 45 aa, Artificial Sequence
  Synthetic Polypeptide
IHSEKSNKSVAMDMLNEVKRCPDELFTTLSAEKQSRFRIISDDHN

SEQ ID NO: 469 - protein, 53 aa, Artificial Sequence
  Synthetic Polypeptide
LESRYDHNQMLLDMLSELSRCPKLLYEKLSEENKKHFQVEADGFLDEIEEEQN

SEQ ID NO: 470 - protein, 56 aa, Artificial Sequence
  Synthetic Polypeptide
LESLRMDDWMLLDMLNELVRCPKPLYDRLREDDRACFRVPVDILPDEDDTDGGGED

SEQ ID NO: 471 - protein, 53 aa, Artificial Sequence
  Synthetic Polypeptide
LENVQTKDWMQLDMLNELVRCPKSLYERLREKDRESFKVPFDIFSDDYNAEEE

SEQ ID NO: 472 - protein, 57 aa, Artificial Sequence
  Synthetic Polypeptide
KDFPIAGSILDREKQPVAGKIGIKVKLLNQQYVSEVDKAVKAHQLKQRKASKPSIQN

SEQ ID NO: 473 - protein, 41 aa, Artificial Sequence
  Synthetic Polypeptide
ANPANYPYIVDTYTHYILENNKVEMFINDKEDSAPLLPVIE

SEQ ID NO: 474 - protein, 38 aa, Artificial Sequence
  Synthetic Polypeptide
KETSNQPFISKTTPHYHITDNKIGFRLGTSKELYPSLE

SEQ ID NO: 475 - protein, 39 aa, Artificial Sequence
  Synthetic Polypeptide
FETGDKPYISQTSPHYHIEKGKIGLRFMPEGQHLWPSPE

SEQ ID NO: 476 - protein, 39 aa, Artificial Sequence
  Synthetic Polypeptide
FETSQEPYISKTAPHYHLENEKIGIKFCSAHNNLFPSLQ

SEQ ID NO: 477 - protein, 60 aa, Artificial Sequence
  Synthetic Polypeptide
IIEEIVPINESNPKEAIVFGGQPTAYLSMNDIHSILYEFFDKWEKKKEKLEKKGEKELRK

SEQ ID NO: 478 - protein, 37 aa, Artificial Sequence
  Synthetic Polypeptide
DDRYVVKTIPSCRMSTLEIPAMAFHMFLFGSKKTEKL

SEQ ID NO: 479 - protein, 44 aa, Artificial Sequence
  Synthetic Polypeptide
IKDGANRIAKYPYNSGFVAHAFISVHELLPLMFYQHLTGKSEDL

SEQ ID NO: 480 - protein, 54 aa, Artificial Sequence
  Synthetic Polypeptide
VGTTRTGRSKYAQDKRLTAEAFLSVHELMPMMFYYFLLREKYSEEVSAERVQGR

SEQ ID NO: 481 - protein, 54 aa, Artificial Sequence
  Synthetic Polypeptide
TDKTCNGRSKFNLGTQFTAEAFLSVHELLPMMFYYLLLTKDYSRKESADKVEGI

SEQ ID NO: 482 - protein, 58 aa, Artificial Sequence
  Synthetic Polypeptide
EIGKELEKKIVGKIQAQIQQIIDKDTNAKILKPYQDGNSTAIDKEKLIKDLKQEQNIL

SEQ ID NO: 483 - protein, 40 aa, Artificial Sequence
  Synthetic Polypeptide
IVDVHNRYKRLFQAMQKEEVTAENIASFGIAESDLPQKIL

SEQ ID NO: 484 - protein, 44 aa, Artificial Sequence
  Synthetic Polypeptide
LKETVRHIQRIYKDFEEERINTIEDLEKANQGRLPLGAFPKQML

SEQ ID NO: 485 - protein, 45 aa, Artificial Sequence
  Synthetic Polypeptide
IKRVIEDVYAVYDAFARDEINTRDELDACLADKGIRRGHLPRQMI

SEQ ID NO: 486 - protein, 45 aa, Artificial Sequence
  Synthetic Polypeptide
IRKEISNIYAIYDAFANNEINSIADLTRRLQNTNILQGHLPKQMI

SEQ ID NO: 487 - protein, 54 aa, Artificial Sequence
  Synthetic Polypeptide
QKLKDEQTVREKEYNDFIAYQDKNREINKVRDRNHKQYLKDNLKRKYPEAPARK

SEQ ID NO: 488 - protein, 50 aa, Artificial Sequence
  Synthetic Polypeptide
DLISGNAHGKDVDAFIRLTVDDMLTDTERRIKRFKDDRKSIRSADNKMGF

SEQ ID NO: 489 - protein, 44 aa, Artificial Sequence
  Synthetic Polypeptide
GLLQNKQPDLSEKAKIKIEKLIAETKLLSHRLNTKLKSSPKLGK

SEQ ID NO: 490 - protein, 44 aa, Artificial Sequence
  Synthetic Polypeptide
AILSQEHKDMEEKIRKKLQEMMADTDHRLDMLDRQTDRKIRIGR

SEQ ID NO: 491 - protein, 44 aa, Artificial Sequence
  Synthetic Polypeptide
SILKGRQKDMGKEAERKIGEMIDDTQRRLDLLCKQTNQKIRIGK

SEQ ID NO: 492 - protein, 51 aa, Artificial Sequence
  Synthetic Polypeptide
EVLYYREKGKVAVWLANDIKRFMPTDFKNEWKGEQHSLLQKSLAYYEQCKE

SEQ ID NO: 493 - protein, 54 aa, Artificial Sequence
  Synthetic Polypeptide
RGFKQISTGKLADFLAKDIVLFQPSVNDGENKITGLNYRIMQSAIAVYDSGDDY

SEQ ID NO: 494 - protein, 59 aa, Artificial Sequence
  Synthetic Polypeptide
RREKLIKTGVLADWLVKDFMRFQPVAYDAQNQPIKSSKANSTEFWFIRRALALYGGEKN

SEQ ID NO: 495 - protein, 59 aa, Artificial Sequence
  Synthetic Polypeptide
KNAGLPKSGVIADWLVRDMMRFQPVAKDASGKPLNNSKANSTEYRMLQRALALFGGEKE

SEQ ID NO: 496 - protein, 59 aa, Artificial Sequence
  Synthetic Polypeptide
RNAGLLKSGKIADWLVNDMMRFQPVQKDQNNIPINNSKANSTEYRMLQRALALFGSENF

SEQ ID NO: 497 - protein, 49 aa, Artificial Sequence
  Synthetic Polypeptide
ELKNLLPEKVFQHLPFKLGGYFFQQKYLYQFYTCYLDKRLEYISGLVQQ

SEQ ID NO: 498 - protein, 60 aa, Artificial Sequence
  Synthetic Polypeptide
EAKQQFKLMFEKARLIGKGTTEPHPFLYKVFARSIPANAVEFYERYLIERKFYLTGLSNE

SEQ ID NO: 499 - protein, 53 aa, Artificial Sequence
  Synthetic Polypeptide
RLEGYFKQTNLIGNTNPHPFLNKFNWKACRNLVDFYQQYLEQREKFLEAIKNQ

SEQ ID NO: 500 - protein, 53 aa, Artificial Sequence
  Synthetic Polypeptide
RLTPYFRQMNLTGGNNPHPFLHETRWESHTNILSFYRSYLRARKAFLERIGRS

SEQ ID NO: 501 - protein, 53 aa, Artificial Sequence
  Synthetic Polypeptide
RLKAYFNQMNLVGNDNPHPFLAETQWEHQTNILSFYRNYLEARKKYLKGLKPQ

SEQ ID NO: 502 - protein, 50 aa, Artificial Sequence
  Synthetic Polypeptide
AENFKSENKVFKKVENECFKFLKKQNYTHKELDARVQSILGYPIFLERGF

SEQ ID NO: 503 - protein, 43 aa, Artificial Sequence
  Synthetic Polypeptide
IKKGNRVDVPFIRRDQNKWKTPAMKTLGRIYSEDLPVELPRQM

SEQ ID NO: 504 - protein, 36 aa, Artificial Sequence
  Synthetic Polypeptide
PWEPYQYCLLLKIPKENRKNLVKGWEQGGISLPRGL

SEQ ID NO: 505 - protein, 35 aa, Artificial Sequence
  Synthetic Polypeptide
DRVENRPFLLLKEPKTDRQTLVAGWKGEFHLPRGI

SEQ ID NO: 506 - protein, 35 aa, Artificial Sequence
  Synthetic Polypeptide
NWKQYQHFLILKVQKTNRNTLVTGWKNSFNLPRGI

SEQ ID NO: 507 - protein, 28 aa, Artificial Sequence
  Synthetic Polypeptide
NRQMRNASSEEIETILDKRLSNSRNEYQ

SEQ ID NO: 508 - protein, 19 aa, Artificial Sequence
  Synthetic Polypeptide
LELHTSDRWKDYLLYKRWQ

SEQ ID NO: 509 - protein, 60 aa, Artificial Sequence
  Synthetic Polypeptide
IEYASPGNKKKIEQLLRDLSLWEAFESKLKVRADRINLAKLKKEILEAQEHPYHDFKSWQ

SEQ ID NO: 510 - protein, 15 aa, Artificial Sequence
  Synthetic Polypeptide
KKKTDLAYLDFLSWK

SEQ ID NO: 511 - protein, 59 aa, Artificial Sequence
  Synthetic Polypeptide
KRKTKIYQQKKNDVFTLLMAKHIFKSVFKQDSIDQFSLEDLYYQSREERLGNQERARQT

SEQ ID NO: 512 - protein, 44 aa, Artificial Sequence
  Synthetic Polypeptide
KESKVIRRYRVQDALLFLLAKKTLTELADFDGERFKLKEIMPDA

SEQ ID NO: 513 - protein, 52 aa, Artificial Sequence
  Synthetic Polypeptide
HLEKKLRLYRNQDVMLWLMTLELTKNHFKELNLNYHQLKLENLAVNVQEADA

SEQ ID NO: 514 - protein, 50 aa, Artificial Sequence
  Synthetic Polypeptide
KFERELRLVKNQDIITWMMCRDLMEENKVEGLDTGTLYLKDIRPNVQEQG

SEQ ID NO: 515 - protein, 50 aa, Artificial Sequence
  Synthetic Polypeptide
KFERELRLIKNQDIVTWLMFKELFNMATVEGLKIGEIHLRDIDTNTANEE

SEQ ID NO: 516 - protein, 40 aa, Artificial Sequence
  Synthetic Polypeptide
GERNTNYIWNKTVDLKLCDGKITVENVKLKNVGDFIKYEY

SEQ ID NO: 517 - protein, 42 aa, Artificial Sequence
  Synthetic Polypeptide
EKGILSEIMPMSFTFEKGGKKYTITSEGMKLKNYGDFFVLAS

SEQ ID NO: 518 - protein, 57 aa, Artificial Sequence
  Synthetic Polypeptide
KLNPLNQTLPMVLPVKVYPATAFGEVQYHKTPIRTVYIREESHTKALKMGNFKALVK

SEQ ID NO: 519 - protein, 56 aa, Artificial Sequence
  Synthetic Polypeptide
SLNVLNRVKPMRLPVVVYRADSRGHVHKEEAPLATVYIEERDTKLLKQGNFKSFVK

SEQ ID NO: 520 - protein, 56 aa, Artificial Sequence
  Synthetic Polypeptide
SNNILNRRIMPMKLPVKTYETDNKGNILKERPLATFYIEETETKVLKQGNFKALVK

SEQ ID NO: 521 - protein, 57 aa, Artificial Sequence
  Synthetic Polypeptide
DQRVQAFLKYEENIEWQAFLIKESKEEENYPYVVEREIEQYEKVRREELLKEVHLIE

SEQ ID NO: 522 - protein, 43 aa, Artificial Sequence
  Synthetic Polypeptide
DKRIGNLLELVGSDIVSKEDIMEEFNKYDQCRPEISSIVFNLE

SEQ ID NO: 523 - protein, 49 aa, Artificial Sequence
  Synthetic Polypeptide
DRRLNGLFSFIKEENDTQKHPISQLRLRRELEIYQSLRVDAFKETLSLE

SEQ ID NO: 524 - protein, 50 aa, Artificial Sequence
  Synthetic Polypeptide
DRRLNGLFSFVDTGGLAMEQYPISKLRVEYELAKYQTARVCVFELTLRLE

SEQ ID NO: 525 - protein, 50 aa, Artificial Sequence
  Synthetic Polypeptide
DRRLNGLFSFAETTDLNLEEHPISKLSVDLELIKYQTTRISIFEMTLGLE

SEQ ID NO: 526 - protein, 60 aa, Artificial Sequence
  Synthetic Polypeptide
EYILEKVKDKEILKKGDNQNFKYYILNGLLKQLKNEDVESYKVFNLNTEPEDVNINQLKQ

SEQ ID NO: 527 - protein, 31 aa, Artificial Sequence
  Synthetic Polypeptide
KWAFDTYPELSARVDREEKVDFKSILKILLN

SEQ ID NO: 528 - protein, 30 aa, Artificial Sequence
  Synthetic Polypeptide
EKLLNKHTSLSSLENEFRALLEEWKKEYAA

SEQ ID NO: 529 - protein, 28 aa, Artificial Sequence
  Synthetic Polypeptide
ESLLTRYPHLPDESFREMLESWSDPLLA

SEQ ID NO: 530 - protein, 27 aa, Artificial Sequence
  Synthetic Polypeptide
KKLIDKYSTLPTDSFRNMLERWLQCKA

SEQ ID NO: 531 - protein, 52 aa, Artificial Sequence
  Synthetic Polypeptide
EATDLEQKAFVLTYIRNKFAHNQLPKKEFWDYCQEKYGKIEKEKTYAEYFAE

SEQ ID NO: 532 - protein, 45 aa, Artificial Sequence
  Synthetic Polypeptide
NKNINKEQSDILRKIRNAFDHNNYPDKGVVEIKALPEIAMSIKKA

SEQ ID NO: 533 - protein, 59 aa, Artificial Sequence
  Synthetic Polypeptide
SSMVTDEHIAFIASVRNAFCHNQYPFYKEALHAPIPLFTVAQPTTEEKDGLGIAEALLK

SEQ ID NO: 534 - protein, 60 aa, Artificial Sequence
  Synthetic Polypeptide
KWPELHGKVRLLIAVRNAFSHNQYPMYDEAVFSSIRKYDPSSPDAIEERMGLNIAHRLSE

SEQ ID NO: 535 - protein, 59 aa, Artificial Sequence
  Synthetic Polypeptide
NRPELKNYVNSLIAVRNAFSHNQYPMYDATLFAEVKKFTLFPSVDTKKIELNIAPQLLE

SEQ ID NO: 536 - DNA, 45 bp, Artificial Sequence
  Synthetic Polynucleotide
cacgcgtgagaacgtaaaaaagcaccctactcaggagcactctca

SEQ ID NO: 537 - DNA, 52 bp, Artificial Sequence
  Synthetic Polynucleotide
agacgtgtgctcttccgatctgctcaggcccaaagcacccatactcaggagc

SEQ ID NO: 538 - DNA, 52 bp, Artificial Sequence
  Synthetic Polynucleotide
agacgtgtgctcttccgatctgggtgaagtacacccatactcaggagcactc

SEQ ID NO: 539 - DNA, 53 bp, Artificial Sequence
  Synthetic Polynucleotide
ccaccgcaaatgcttctaggcggactatgacttagttgcgttacaccctttct

SEQ ID NO: 540 - DNA, 55 bp, Artificial Sequence
  Synthetic Polynucleotide
tcgtccaccgcaaatgcttctaggcggcggactatgacttagttgcgttacaccc

SEQ ID NO: 541 - DNA, 43 bp, Artificial Sequence
  Synthetic Polynucleotide
catcctggcctcgctgtccaccttccagcagatgtggatcagc

SEQ ID NO: 542 - DNA, 48 bp, Artificial Sequence
  Synthetic Polynucleotide
gacgttgctcttccgatctccccacgaccgcagatgtggatcagcaag

SEQ ID NO: 543 - DNA, 48 bp, Artificial Sequence
  Synthetic Polynucleotide
ggatcctagcaaagctgttgaagaagatgactttgtggtggggttctg

SEQ ID NO: 544 - DNA, 55 bp, Artificial Sequence
  Synthetic Polynucleotide
ttcagacgtgtgctcttccgatctccccaccaacactttgtggtggggttctgga